(19) United States
(12) Patent Application Publication
(10) Pub. No.: US 2025/0177582 A1
(43) Pub. Date: Jun. 5, 2025

(54) HIGH-AFFINITY LIGANDS OF FIBROBLAST ACTIVATION PROTEIN FOR TARGETED DELIVERY APPLICATIONS

(71) Applicant: PHILOCHEM AG, Otelfingen (CH)

(72) Inventors: Sara PUGLIOLI, Otelfingen (CH); Nicholas FAVALLI, Otelfingen (CH); Gabriele BASSI, Otelfingen (CH); Andrea GALBIATI, Otelfingen (CH); Aureliano ZANA, Otelfingen (CH); Samuele CAZZAMALLI, Otelfingen (CH); Dario NERI, Otelfingen (CH)

(21) Appl. No.: 18/834,172

(22) PCT Filed: Jan. 30, 2023
(86) PCT No.: PCT/EP2023/052172

§ 371 Date: Jul. 29, 2024

(30) Foreign Application Priority Data
Jan. 30, 2022 (EP) 22154130.3
Sep. 2, 2022 (EP) 22193759.2

(51) Int. Cl. A61K 51/08; A61K 47/54; A61K 47/62; A61K 49/00; A61P 35/00

(52) U.S. Cl. CPC A61K 51/088; A61K 47/545; A61K 47/62; A61K 49/0052; A61K 49/0056; A61P 35/00

(57) ABSTRACT

The present invention relates to ligands of Fibroblast Activation Protein (FAP) for the active delivery of various payloads (e.g. cytotoxic drugs, radionuclides, fluorophores, proteins and immunomodulators) at the site of disease. In particular, the present invention relates to the development of improved FAP ligands for targeting applications, in particular diagnostic methods and/or methods for therapy or surgery in relation to a disease or disorder, such as cancer, inflammation or another disease characterized by overexpression of FAP.

### INTRODUCTION

### Field

The present invention relates to ligands of Fibroblast Activation Protein (FAP) for the active delivery of various payloads (e.g. cytotoxic drugs, radionuclides, fluorophores, proteins and immunomodulators) at the site of disease. In particular, the present invention relates to the development of improved FAP ligands for targeting applications, in particular diagnostic methods and/or methods for therapy or surgery in relation to a disease or disorder, such as cancer, inflammation or another disease characterized by overexpression of FAP.

### BACKGROUND OF THE INVENTION

Chemotherapy is still widely applied for the treatment of cancer patients and of other diseases. Conventional anti-cancer chemotherapeutic agents act on basic mechanisms of cell survival and cannot distinguish between healthy cells and malignant cells. Moreover, those drugs do not accumulate efficiently to the site of the disease upon systemic administration. Unspecific mechanism of actions and inefficient localization at the tumour site account for unsustainable side-effects and poor therapeutic efficacy of conventional chemotherapy.

The development of targeted drugs, able to selectively localize at the site of the disease after systemic administration, is highly desirable. A strategy to generate such drugs is represented by the chemical conjugation of a therapeutic payload, like cytotoxic drugs or radionuclides, to a ligand specific to a marker of a disease. Disease-specific monoclonal antibodies, peptides and small ligands have been considered as ligands of choice for the development of targeted drug products. The use of small ligands for targeting applications has several advantages compared to bigger molecules like peptides and antibodies: more rapid and efficient tumour penetration, lower immunogenicity and lower manufacturing costs.

Small organic ligands specific to prostate-specific membrane antigen, folate receptor and carbonic anhydrase IX have shown excellent biodistribution profiles in preclinical models of cancer and in patients. These ligands have been conjugated to cytotoxic drugs and to radionuclides to generate small molecule-drug conjugate and small molecule-radio conjugate products (SMDCs and SMRCs) for the treatment of cancer. 177-Lutetium-PSMA-617 represents an example of a late stage SMRC which is now being investigated in a phase III trial for the treatment of metastatic castrate-resistant prostate cancer (mCRPC) patients (VISION trial).

Fibroblast activation protein (FAP) is a membrane-bound gelatinase which promotes tumour growth and progression and is overexpressed in cancer-associated fibroblasts. FAP represents an ideal target for the development of targeted SMDCs and SMRCs due to its low expression in normal organs.

The present applicants describe in WO2021160825 ligands of Fibroblast Activation Protein (FAP) termed “ESV6” for the active delivery of various payloads (e.g. cytotoxic drugs, radionuclides, fluorophores, proteins and immunomodulators) at the site of disease. WO2019154886 and WO2019154859 describe heterocyclic compounds as fibroblast activation protein-alpha inhibitors used to treat different cancer types.

WO2019118932 describes substituted N-containing cyclic compounds as fibroblast activation protein alpha inhibitors used to treat different pathological conditions. WO2019083990 describes imaging and radiotherapeutic targeting fibroblast-activation protein-alpha (FAP-alpha) compounds as FAP-alpha inhibitors used for imaging disease associated with FAP-alpha and to treat proliferative diseases, and notes that the 4-isoquinolinoyl and 8-quinolinoyl derivatives described therein are characterized by very low FAP-affinity.

WO2013107820 describes substituted pyrrolidine derivatives used in the treatment of proliferative disorders such as cancers and diseases indicated by tissue remodelling or chronic inflammation such as osteoarthritis. WO2005087235 describes pyrrolidine derivatives as dipeptidyl peptidase IV inhibitors to treat Type II diabetes. WO2018111989 describes conjugates comprising fibroblast activation protein (FAP) inhibitor, and e.g. near infrared (NIR) dye, useful for removing cancer-associated fibroblasts, imaging population of cells in vitro, and treating cancer. Tsutsumi et al. (J Med Chem 1994) describe the preparation and in vitro prolyl endopeptidase (PEP) inhibitory activity of a series of a-keto heterocyclic compounds. Hu et al. (Bioorg Med Chem Lett 2005) describe the structure-activity relationship of various N-alkyl Gly-boro-Pro derivatives against FAP and other two dipeptidyl peptidases. Edosada et al. (J Biol Chem 2006) describe the dipeptide substrate specificity of FAP and the development of a Ac-Gly-BoroPro FAP-selective inhibitor. Gilmore et al. (Biochem Biophys Res Commun 2006) describe the design, synthesis, and kinetic testing of a series of dipeptide proline diphenyl phosphonates, against DPP-IV and FAP. Tran et al. (Bioorg Med Chem Lett 2007) describe the structure-activity relationship of various N-acyl-Gly-, N-acyl-Sar-, and N-blocked-boroPro derivatives against FAP. Tsai et al. (J Med Chem 2010) describe structure-activity relationship studies that resulted in a number of FAP inhibitors with excellent selectivity over DPP-IV, DPP-II, DPP8, and DPP9. Ryabtsova et al. (Bioorg Med Chem Lett 2012) describe the synthesis and the evaluation of FAP inhibition properties of a series of N-acylated glycyl-(2-cyano)pyrrolidines. Poplawski et al. (J Med Chem 2013) describe N-(pyridine-4-carbonyl)-D-Ala-boroPro as a potent and selective FAP inhibitor. Jansen et al. (ACS Med Chem Lett 2013) describe FAP inhibitors based on the N-(4-quinolinoyl)-Gly-(2-cyanopyrrolidine) scaffold. Jansen et al. (Med Chem Commun 2014) the structure-activity relationship of FAP inhibitors based on the linagliptin scaffold. Jansen et al. (Med Chem Commun 2014) describe xanthine-based FAP inhibitors with low micromolar potency. Jansen et al. (J Med Chem 2014) describe the structure-activity relationship of FAP inhibitors based on the N-4-quinolinoyl-Gly-(2S)-cyanoPro scaffold. Jackson et al. (Neoplasia 2015) describe the development of a pseudopeptide inhibitor of FAP. Meletta et al. (Molecules 2015) describe the use of a boronic-acid based FAP inhibitor as non-invasive imaging tracers of atherosclerotic plaques. Dvořáková et al. (J Med Chem 2017) describe the preparation of a polymer conjugate containing a FAP-specific inhibitor for targeting applications. Loktev et al. (J Nucl Med 2018) describe the development of an iodinated and a DOTA-coupled radiotracer based on a FAP-specific enzyme inhibitor. Lindner et al. (J Nucl Med 2018) describe the modification and optimization of FAP inhibitors for use as theranostic tracers. Giesel et al. (J Nucl Med 2019) describe the clinical imaging performance of quinoline-based PET tracers that act as FAP inhibitors.

Nevertheless, there remains a demand for further improving ligands of Fibroblast Activation Protein (FAP).

### Problems to be Solved by the Invention

The present invention aims at the problem of providing improved binders (ligands) of fibroblast activation protein (FAP) suitable for targeting applications. The binders should be suitable for inhibition of FAP and/or targeted delivery of a payload, such as a therapeutic or diagnostic agent, to a site afflicted by or at risk of disease or disorder characterized by overexpression of FAP.

### SUMMARY OF THE INVENTION

The present inventors have found novel organic ligands of fibroblast activation protein (FAP) (“ESV6-11”) suitable for targeting applications. The compounds according to the present invention (also referred to as ligands or binders) comprise at least one binding moiety A independently represented by the following structure:

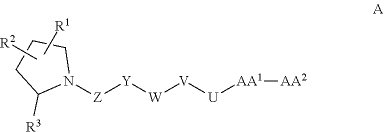

wherein AA¹ is arginine, a derivative or analog thereof having the following structure:

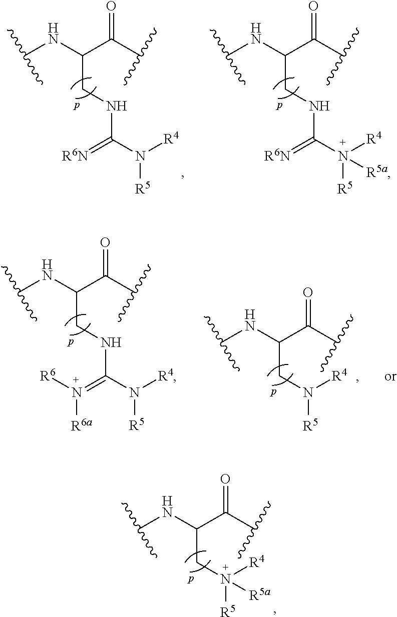

wherein each of R¹ and R² is independently selected from the group consisting of H, OH, halogen, C₁₋₆alkyl, —O—C₁₋₆alkyl, and —S—C₁₋₆alkyl, N₃, preferably H or halogen, more preferably F;
R³ is independently selected from H, —CN, —B(OH)₂, —C(O)alkyl, —C(O)haloalkyl, —C(O)aryl-, —C═C—C(O)aryl, —C═C—S(O)₂aryl, —CO₂H, —SO₃H, —SO₂NH₂, —PO₃H₂, and 5-tetrazolyl, preferably —CN, —B(OH)₂, or —CO₂H, more preferably —CN;
each of R⁴, R⁵, R^{5a}, R⁶, and R^{6a} is independently H, OH, optionally substituted C₁-C₆ alkyl, optionally substituted C₁-C₆ haloalkyl, optionally substituted —C(O)C₁-C₅ alkyl, or optionally substituted —C(O)C₁-C₅ haloalkyl, and wherein two of them can be joined together or any one of them can be joined to the (CH₂)ₚ moiety to form a ring structure, preferably wherein at least one of R⁴, R⁵, R^{5a}, R⁶, and R^{6a} is not H; and p is 1 to 5, preferably 2, 3 or 4, more preferably 3;
AA² is absent, or represents

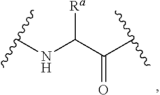

wherein R^{a} is selected from H, optionally substituted C₁₋₆ alkyl, (C₃-C₁₀ carbocyclyl)C₁₋₆ alkyl, (C₆-C₁₀ aryl)C₁₋₆ alkyl, (C₁-C₁₀ heterocyclyl)C₁₋₆ alkyl, C₂₋₆ alkenyl, C₂₋₆ alkynyl, and C₆-C₁₀ aryl, in each of which optionally one or more of the carbon atoms can be replaced by heteroatoms; preferably, R^{a} is a side chain derived from a proteinogenic or non-proteinogenic amino acid; more preferably, R^{a} is H, optionally substituted C₁₋₆ alkyl, (C₆-C₁₀ aryl)C₁₋₄ alkyl, or (C₂₋₆ alkynyl)C₁₋₄ alkyl;
Z is C(O), 0, CH₂, NR′, C═O, C═S, C═NR′, HCR′ and R′CR′, with the proviso that two Os are not directly adjacent to each other, and is preferably C(O);
Y is a single bond or a linking group, preferably an optionally substituted C₁₋₁₆ aliphatic group, in which optionally one or more carbon atoms can be replaced by heteroatoms, and which can be saturated optionally contain one or more double or triple bonds;
W is a carbocyclic or heterocyclic group;
V is a single bond or a linking group, preferably an optionally substituted C₁₋₁₆ aliphatic group, in which optionally one or more carbon atoms can be replaced by heteroatoms, and which can be saturated optionally contain one or more double or triple bonds;
U is single bond or C(O), 0, CH₂, NR′, C═O, C═S, C═NR′, HCR′ and R′CR′, preferably C(O); and
each R′ is independently H or selected from H, SH, NH₂, halogen, cyano, carboxy, C₁₋₆-alkyl, O(C₁₋₆ alkyl), S(C₁₋₆-alkyl), C₂₋₆ alkenyl, C₂₋₆ alkynyl, C₁₋₆ heteroalkenyl, C₁₋₆ heteroalkynyl, C₃₋₁₀ cycloalkenyl, C₁₋₁₀ cycloheteroalkenyl, C₆₋₁₀ aryl, and (C₆₋₁₀ aryl)C₁₋₆ alkyl, each of which being optionally substituted with from 1 to 3 substituents selected from —OH, oxo and halo, and optionally connected to an atom in Z, Y, W or V.

The present invention further provides a pharmaceutical composition comprising said compound and a pharmaceutically acceptable excipient.

The present invention further provides said compound or pharmaceutical composition for use in a method for treatment of the human or animal body by surgery or therapy or a diagnostic method practised on the human or animal body; as well as a method for treatment of the human or animal body by surgery or therapy or a diagnostic method practised on the human or animal body comprising administering a therapeutically or diagnostically effective amount of said compound or pharmaceutical composition to a subject in need thereof.

The present invention further provides said compound or pharmaceutical composition for use in a method for therapy or prophylaxis of a subject suffering from or having risk for a disease or disorder; as well as a method for treatment therapy or prophylaxis of a disease or disorder comprising administering a therapeutically or diagnostically effective amount of said compound or pharmaceutical composition to a subject suffering from or having risk for said disease or disorder.

The present invention further provides said compound or pharmaceutical composition for use in a method for guided surgery practised on a subject suffering from or having risk for a disease or disorder; as well as a method for guided surgery comprising administering a therapeutically or diagnostically effective amount of said compound or pharmaceutical composition to a subject suffering from or having risk for a disease or disorder.

The present invention further provides said compound or pharmaceutical composition for use in a method for diagnosis of a disease or disorder, the method being practised on the human or animal body and involving a nuclear medicine imaging technique, such as Positron Emission Tomography (PET); as well as a method for diagnosis of a disease or disorder, the method being practised on the human or animal body and involving a nuclear medicine imaging technique, such as Positron Emission Tomography (PET), and comprising administering a therapeutically or diagnostically effective amount of said compound or pharmaceutical composition to a subject in need thereof.

The present invention further provides said compound or pharmaceutical composition for use in a method for targeted delivery of a therapeutic or diagnostic agent to a subject suffering from or having risk for a disease or disorder; as well as a method for targeted delivery of a therapeutically or diagnostically effective amount of said compound or pharmaceutical composition to a subject suffering from or having risk for a disease or disorder.

Preferably, the aforementioned disease or disorder is characterized by overexpression of FAP and is independently selected from cancer, inflammation, atherosclerosis, fibrosis, tissue remodelling and keloid disorder, preferably wherein the cancer is selected from the group consisting of breast cancer, pancreatic cancer, small intestine cancer, colon cancer, multi-drug resistant colon cancer, rectal cancer, colorectal cancer, metastatic colorectal cancer, lung cancer, non-small cell lung cancer, head and neck cancer, ovarian cancer, hepatocellular cancer, oesophageal cancer, hypopharynx cancer, nasopharynx cancer, larynx cancer, myeloma cells, bladder cancer, cholangiocarcinoma, clear cell renal carcinoma, neuroendocrine tumour, oncogenic osteomalacia, sarcoma, CUP (carcinoma of unknown primary), thymus cancer, desmoid tumours, glioma, astrocytoma, cervix cancer, skin cancer, kidney cancer and prostate cancer. More preferably, the disease or disorder is selected from melanoma and renal cell carcinoma.

### BRIEF DESCRIPTION OF THE DRAWINGS

FIG. 1: Schematic representation of the affinity maturation of small organic ligands of Fibroblast Activation Protein underlying the present invention

FIG. 2: Inhibition assay performed overtime with compound 8 at 22.6° C. with different concentrations human FAP: a) [hFAP]=2.5 nM, b) [hFAP]=625 pM, c) [hFAP]=312 pM, d) [hFAP]=156 pM, e) [hFAP]=78 pM, f) [hFAP]=39 pM. The concentration of substrate (Z-Gly-Pro-AMC) was kept constant=20 μM. Dashed lines corresponds to sets of data with unstable fitting. The fitted curves yielded IC₅₀ values ranging between 21 nM to 6 nM. Error bars display SEM.

FIG. 3: Inhibition assay performed with compounds 1, 2, 3, 4, 5, 6, 8 against a) 66 pM human FAP and b) 66 pM murine FAP. The concentration of substrate (Z-Gly-Pro-AMC) was kept constant=20 μM. The assay was incubated for 48 hours at 22.6° C. The fitted curves yielded IC₅₀ values reported in Tables 1 and 2. Error bars display SEM.

FIG. 4: a) Inhibition assay performed with compounds 8, 9 and 10 against 66 pM human FAP. The assay was incubated for 17 hours at 25° C. b) Inhibition assay performed with compound 9 against 66 pM murine FAP. The assay was incubated for 17 hours at 25° C. In both assays the concentration of substrate (Z-Gly-Pro-AMC) was kept constant=20 μM. The fitted curves yielded IC₅₀ values reported in Tables 1 and 2. Error bars display SEM.

FIG. 5: Inhibition assay performed with compounds 11, 12, 14-16 (a) and 17-19 (b).

FIG. 6: In vivo biodistribution experiments: C9 ([¹⁷⁷Lu]—Lu-ESV6-11-DOTAGA) accumulated selectively in FAP-expressing HT-1080 tumors already 1 h after intravenous injection (23.6% ID/g), with a sustained uptake in the lesions over the first 72 hours. The compound did not accumulate in FAP-negative tumors subcutaneously implanted in the same animal. No substantial uptake was observed in healthy organs (e.g., 7-to-1 in kidney after 1 h and 4 h).

FIG. 7: Inhibition assay performed with compounds 37 and 38 against 66 pM human FAP. The assay was incubated for 24 hours at 25° C. In both assays, the concentration of substrate (Z-Gly-Pro-AMC) was kept constant=20 μM. The fitted curves yielded IC₅₀ values reported in Table 2. Error bars display SEM.

FIG. 8: In vivo biodistribution experiments with [¹⁷⁷Lu]-compound 42 ([¹⁷⁷Lu]—Lu—Bi-ESV6-11-DOTAGA).

The compound is selectively accumulated in FAP-expressing HT-1080 tumors with a sustained uptake in the lesions over the first 72 hours.

FIG. 9: Flow cytometry analysis with compounds 36 and 29 (100 nM) performed against FAP positive SKRC-52.hFAP and FAP negative SKRC-52.wt. Cell populations are visualized as histograms. The detected fluorescence intensity and the population of cells normalized on the number of events (Normalized to mode) are reported in the x and y axes, respectively.

FIG. 10: Confocal microscopy pictures of FAP positive SKRC-52.hFAP and FAP negative SKRC-52.wt. exposed to compounds 36 and 29 (150 nM). The scale bar is set at 10 μM.

FIG. 11: Structure and LC traces of compound 2 (A), compound 3 (B) and compound 4 (C).

FIG. 12: Structure and LC traces of compound 9 (A), compound 10 (B), and compound 11 (C).

FIG. 13: Structure and LC traces of compound 14 (A), compound 15 (B), and compound 16 (C).

FIG. 14: Structure and LC traces of compound 17 (A), compound 18 (B), and compound 19 (C).

FIG. 15: Structure and LC traces of compound 28 (A), compound 29 (B), and compound 36 (C).

FIG. 16: Structure and LC traces of compound 32 (A), compound 37 (B) and compound 38 (C).

FIG. 17: Structure and LC traces of compound 39 (A) and 35 (B).

FIG. 18: Structure and LC trace of compound 42 (A) and 27 (B).

FIG. 19. A: Chemical structures and corresponding IC50_{(hFaP)} values of compounds according to the present invention. B: Chemical structures and corresponding IC50 values of compounds where the propargyl moiety at position AA² was replaced by different groups (Me, iPr, Bn) using both R and S stereoisomers. The sites of modification have been highlighted with arrows. Each measurement was performed with a FAP concentration lower than the expected IC50. Depending on FAP concentration, the fluorescence was measured between 10 minutes and 48 h (at room temperature) after the addition of the fluorogenic substrate (Z-Gly-Pro-AMC). IC50 values and standard errors were calculated using “[Inhibitor] vs. response—Variable slope (four parameters)” fitting (Graphpad Prism).

FIG. 20: In vitro validation of affinity matured FAP ligands. The histogram shows a comparison between compound 8a (comparative) and 28 performed by inhibition assay with human FAP, and the cross-reactivity of compound 28 against feline FAP (fFAP), equine FAP (eFAP), ovine FAP (oFAP) and murine FAP (mFAP). Bars represent average of IC50 values. Error bars represent the standard error of the mean (SEM).

FIG. 21. A: Mouse serum stability of 28 reported at time 0, 1 h, 4 h, 8 h, 24 h, 30 h and 48 h. B: Human serum stability of compound 38 reported at time 0, 4 h, 24 h, 48 h and 72 h. Compound 28 showed particularly favourable in vitro stability in mouse serum, with a half-life of more than 48 hours.

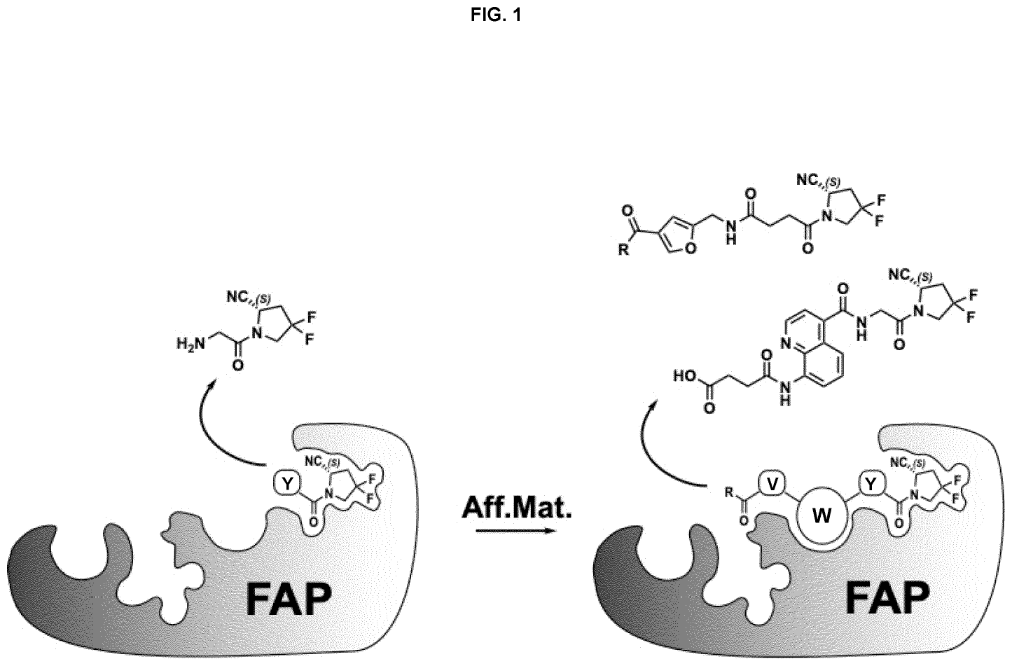

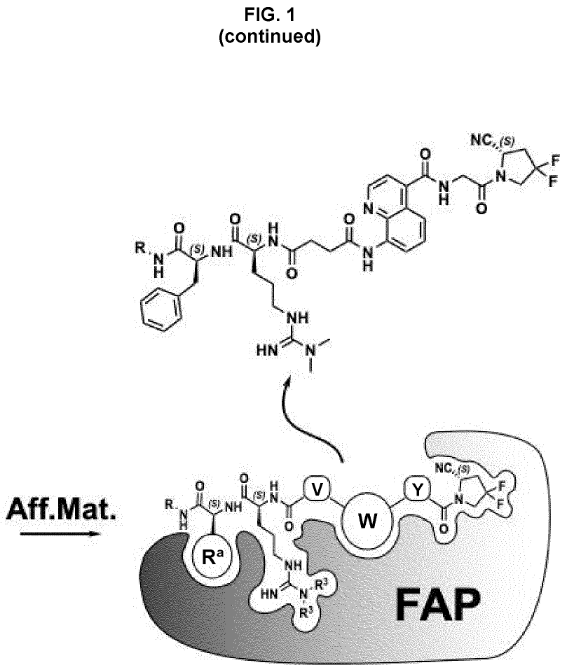

### DETAILED DESCRIPTION OF THE INVENTION

The present inventors have identified small molecule binders of fibroblast activation protein (FAP) which are suitable for targeting applications. The binders according to the invention provide high inhibition of FAP, high affinity for FAP and/or are suitable for targeted delivery of a payload, such as a therapeutic or diagnostic agent, to a site afflicted by or at risk of disease or disorder characterized by overexpression of FAP. The binders according to the present invention form a stable complex with FAP, display an increased affinity, increased inhibitory activity, a slower rate of dissociation from the complex, and/or prolonged residence at a disease site.

Without wishing to be bound by theory, it is considered that adding arginine or derivative/analog thereof at position AA1 may improve proline-based binders of the general formula A:

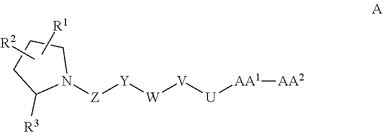

As explained above, the present invention aims at providing FAP binders that may be better than proline-based FAP binders FAP binders known from the prior art, such as compound 8.

The present inventors surprisingly recognized that introducing a “tail” based on a positively charged moiety, including positively charged amino acids, such as arginine or derivative/analog thereof at position AA1 may play a role in improving FAP binders, and tried to add them to compound 8 to obtain create compound 9. The FAP inhibition potency increased strongly.

The present inventors found that the FAP inhibition potency can be increased even further by varying the amino acid at position AA2. Particularly strong FAP inhibition can be achieved when the amino acid at that position is d- or 1-phenylalanine or valine (compounds 11, 17). Such compounds are believed to be the best FAP binder described so far.

Yet further, the present inventors found that this effect is not limited to compounds having a quinoline ring as group W, but can be extended also to other carbocyclic or heterocyclic groups in that position. For instance, starting from compound 5 having a furan ring, and by introducing “tail” based on arginine or derivative/analog thereof resulted in a significant improvement of FAP binding (compound 4). Conversely, removing the furan as group W resulted in worsening the FAP binding affinity (compound 6).

This is confirmed by further compounds, as shown, e.g., in Table 2.

TABLE 1
IC₅₀ values.

| | | IC₅₀ vs | IC₅₀ vs |
|---|---|---|---|
| | | human | murine |
| Compound | | FAP | FAP |
| ID | Structure | (M) | (M) |
| Gly-ProCN | 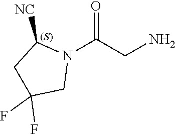 | 2.407e-005 | |
| 1 | 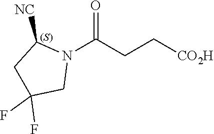 | 0.0002490 | 0.0001999 |
| 5 | 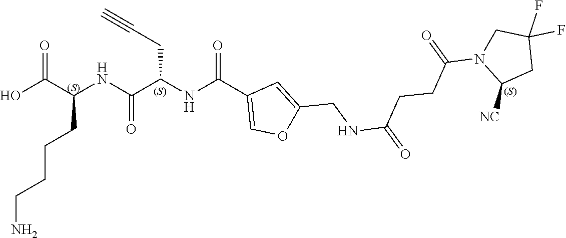 | | 3.899e-006 |
| 6 | 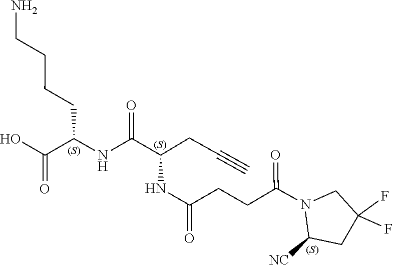 | 1.720e-005 | 2.271e-005 |
| 8 | 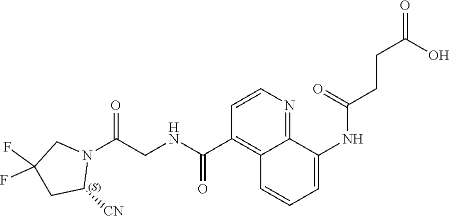 | 1.086e-008 | 1.613e-008 |
| 8a | 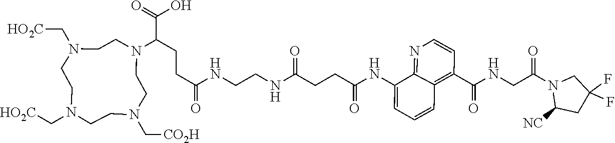 [SMILES: N#C[C@@H]1CC(F)(F)CN1C(=O)CNC(=O)c1ccnc2c(NC(=O)CCC(=O)NCCNC(=O)CCC(C(=O)O)N3CCN(CC(=O)O)CCN(CC(=O)O)CCN(CC(=O)O)CC3)cccc12] | | |

TABLE 2
IC₅₀ values and preferred compounds

| | | IC₅₀ vs | IC₅₀ vs |
|---|---|---|---|
| | | human | murine |
| Compound | | FAP | FAP |
| ID | Structure | (M) | (M) |
| 2 | 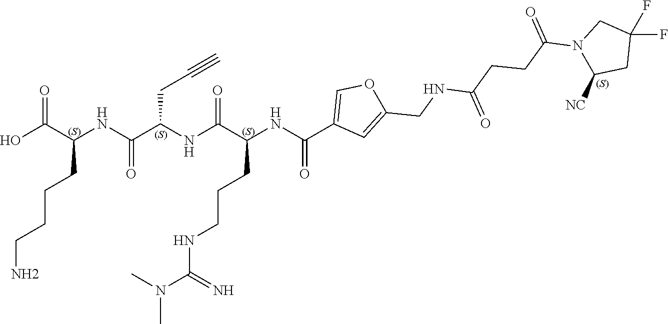 | 4.408e-008 | 2.920e-008 |
| 3 | 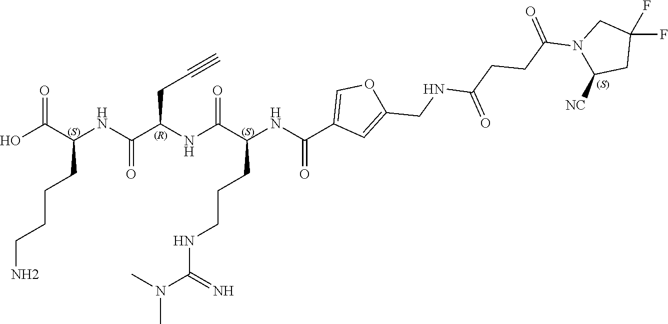 | 6.001e-008 | 3.845e-008 |
| 4 | 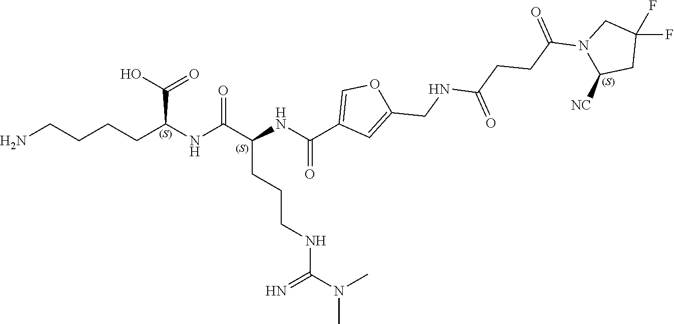 | 1.782e-007 | 1.540e-007 |
| 4a | 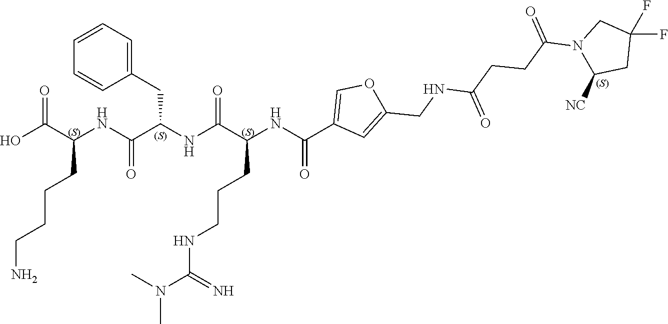 | | |
| 4b | 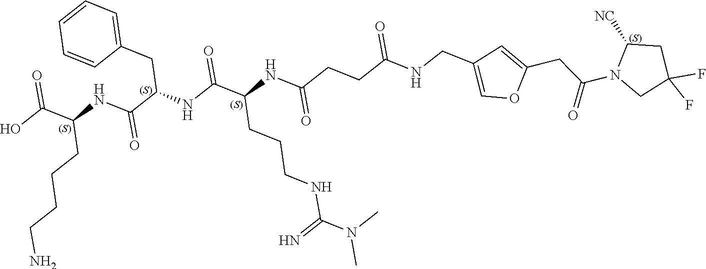 | | |
| 4c | 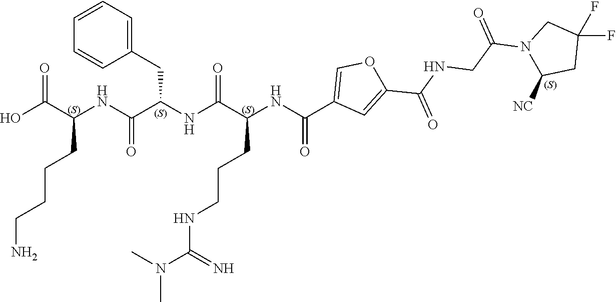 | | |
| 4d | 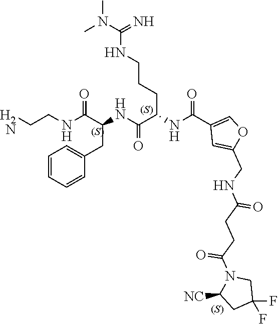 | | |
| 9 | 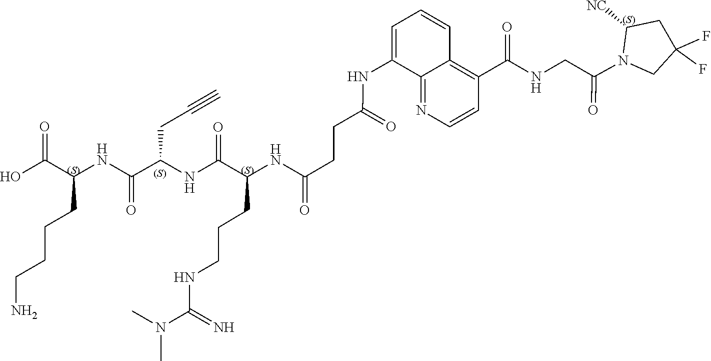 | 4.387e-010 | 1.151e-010 |
| 10 | 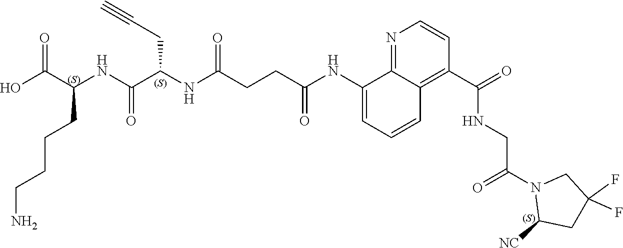 | 4.380e-009 | |
| 11 | 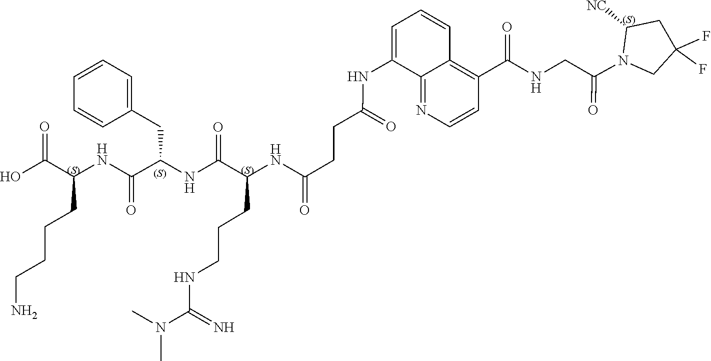 | 1.560e-010 | |
| 12 | 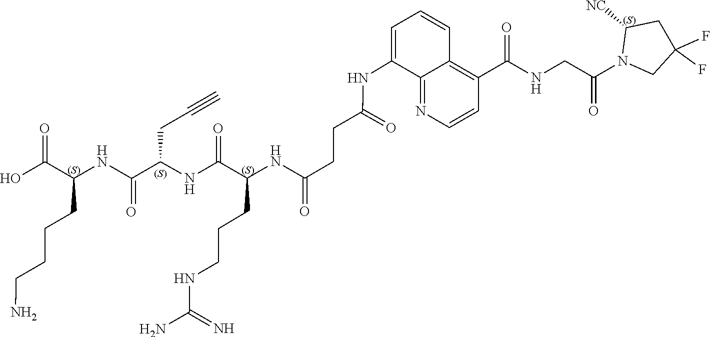 | 1.290e-009 | |
| 13 | 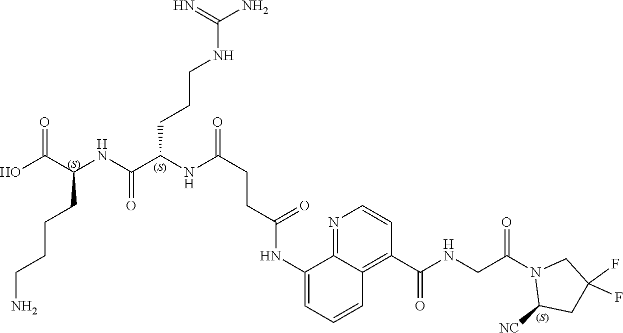 | | |
| 14 | 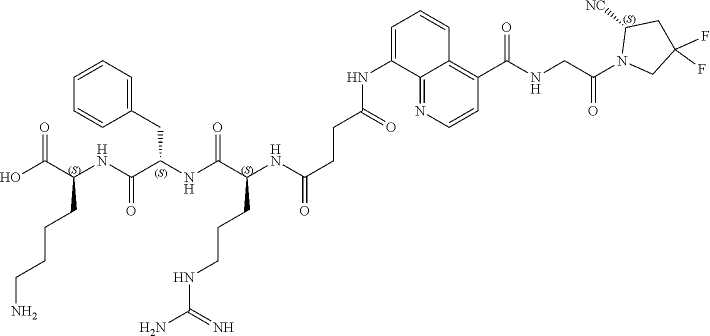 | 3.529e-010 | |
| 15 | 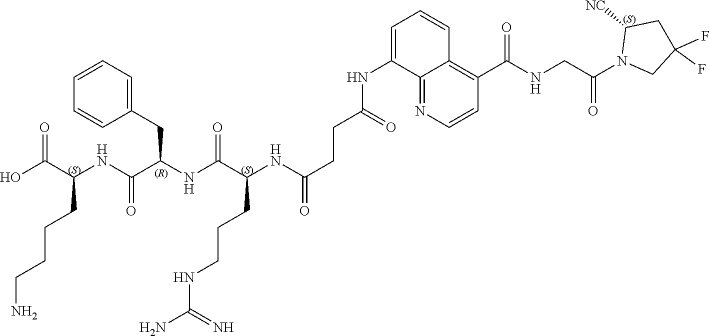 | 6039e-10 | |
| 16 | 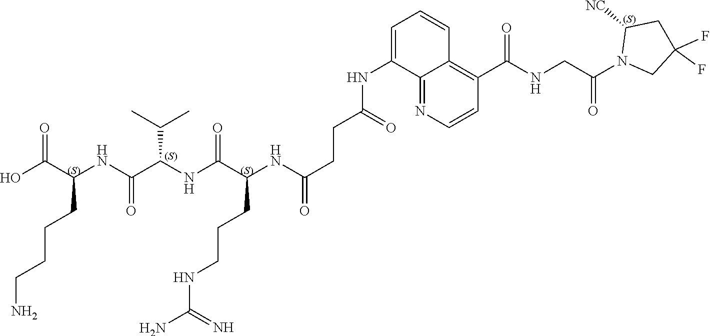 | 1.660e-009 | |
| 17 | 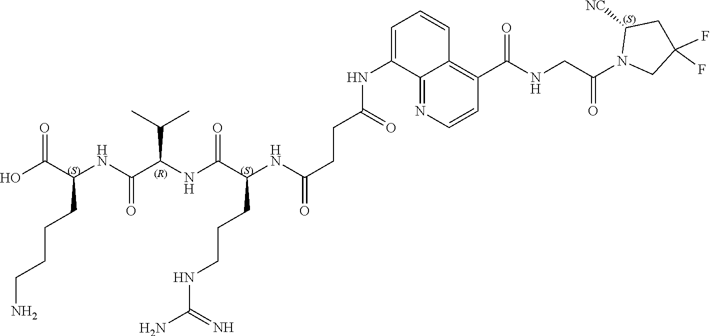 | 6.968e-010 | |
| 18 | 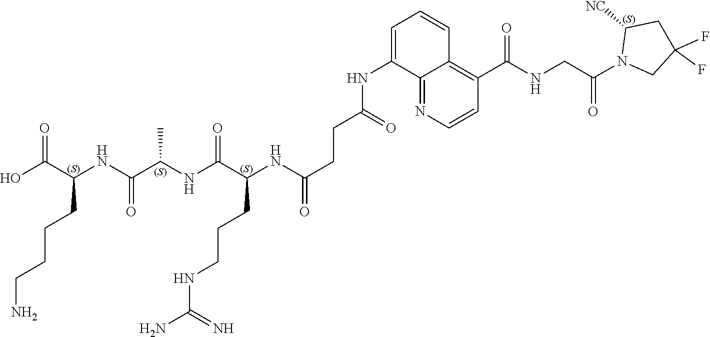 | 1.545e-009 | |
| 19 | 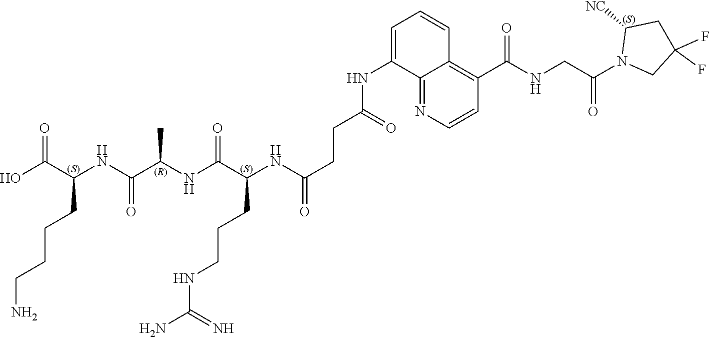 | 9.909e-010 | |
| 25 | 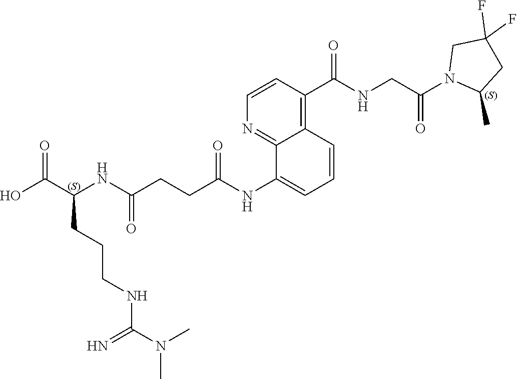 | | |
| 26 | 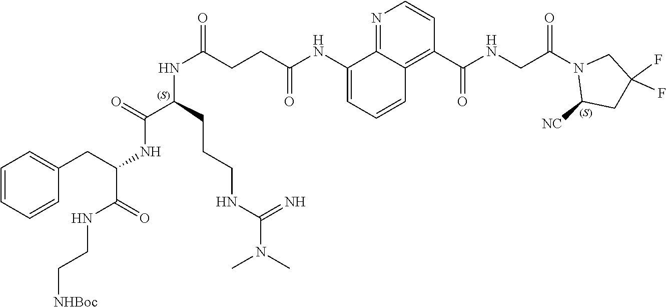 | 7.481e-011 | |
| 27 | 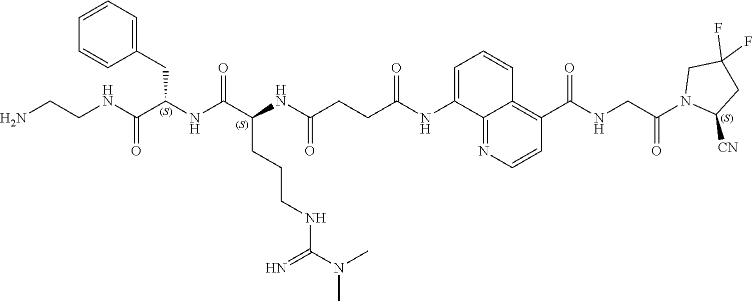 | 1.017e-009 | |
| 28 | 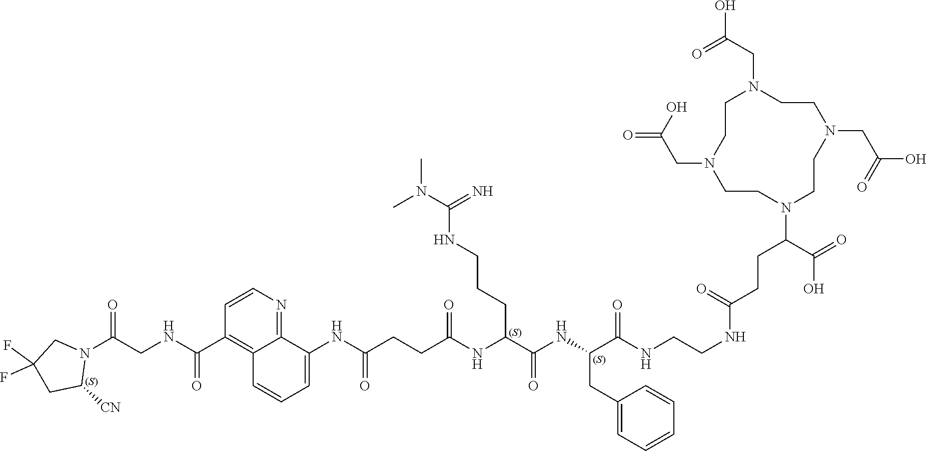 | 3.004e-011 | 4.793e-012 |
| 29 | 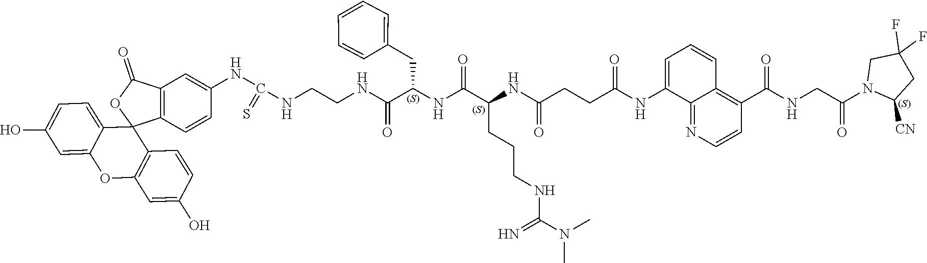 | | |
| 30 | 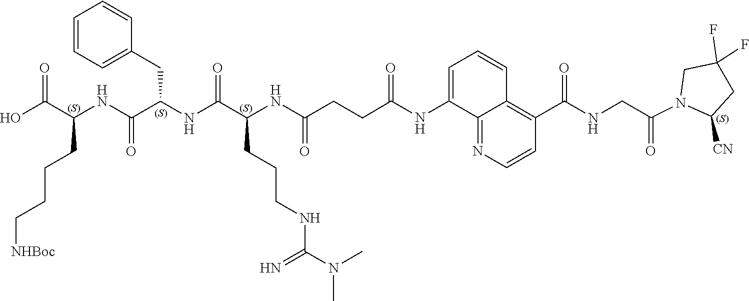 | | |
| 31 | 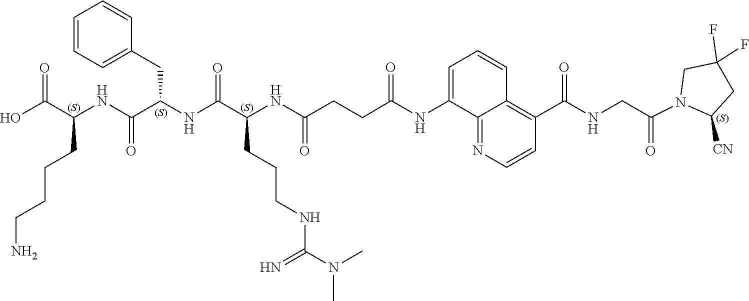 | | |
| 32 | 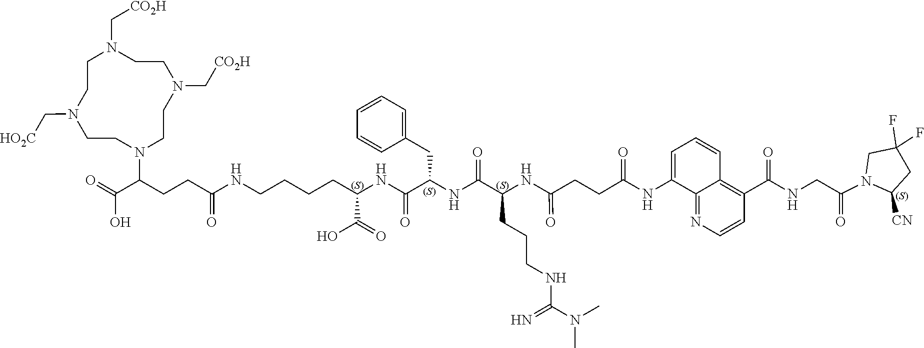 | | |
| 33 | 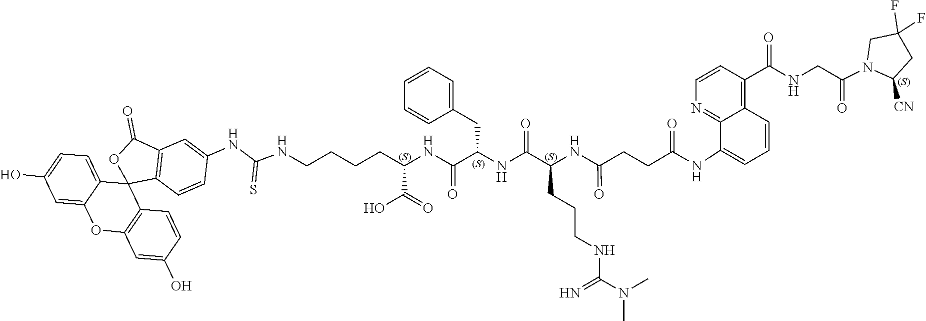 | | |
| 34 | 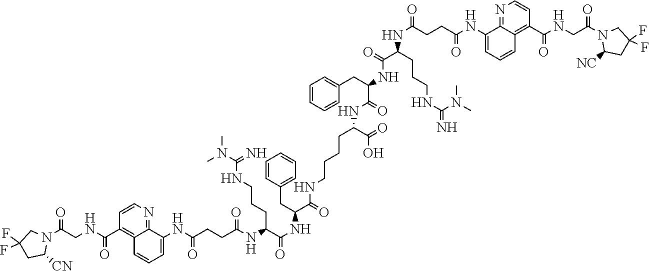 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)[C@@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)O] | | |
| 35 | 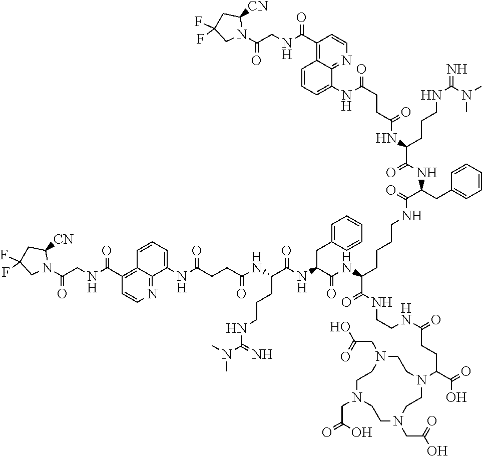 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)NCCNC(=O)CCC(C(=O)O)N1CCN(CC(=O)O)CCN(CC(=O)O)CCN(CC(=O)O)CC1] | | |
| 36 | 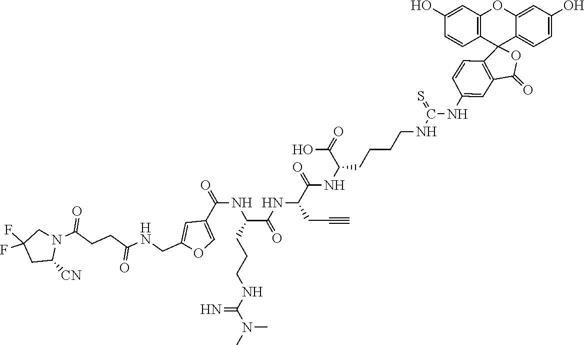 [SMILES: C#CC[C@H](NC(=O)[C@H](CCCNC(=N)N(C)C)NC(=O)c1coc(CNC(=O)CCC(=O)N2CC(F)(F)C[C@H]2C#N)c1)C(=O)N[C@@H](CCCCNC(=S)Nc1ccc2c(c1)C(=O)OC21c2ccc(O)cc2Oc2cc(O)ccc21)C(=O)O] | | |
| 37 | 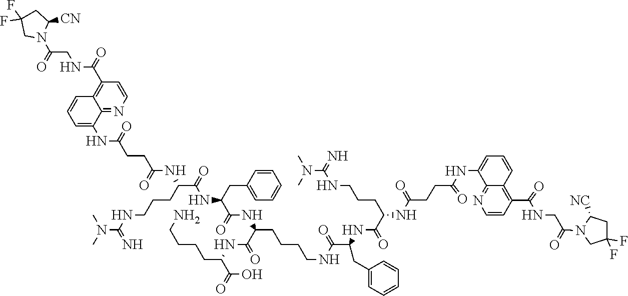 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)N[C@@H](CCCCN)C(=O)O] | | |
| 38 | 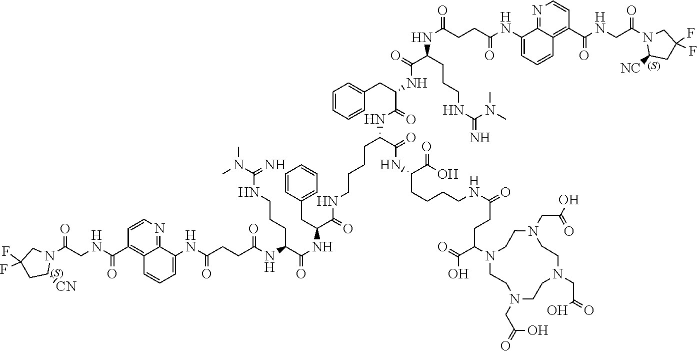 | | |
| 39 | 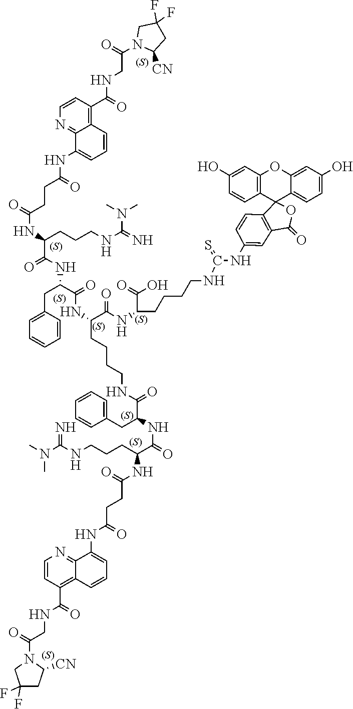 | | |
| 40 | 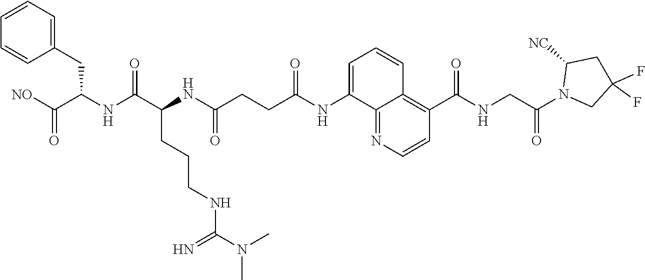 | | |
| 41 | 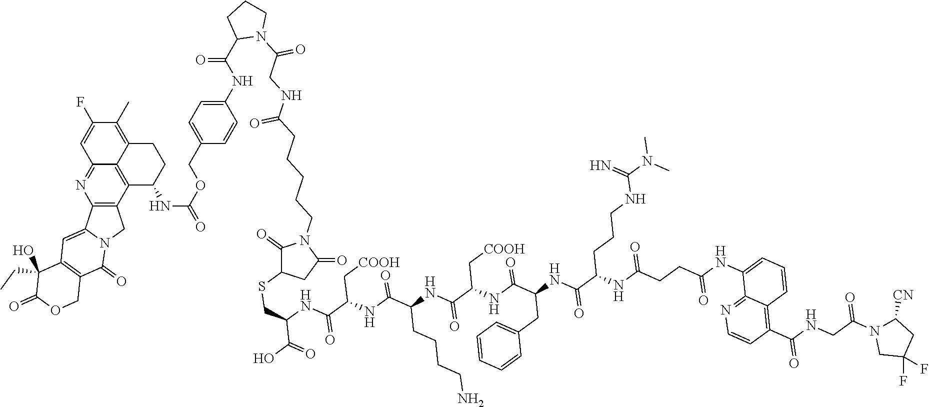 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)C2CCCN2C(=O)CNC(=O)CCCCCN2C(=O)CC(SC[C@@H](NC(=O)[C@H](CC(=O)O)NC(=O)[C@H](CCCCN)NC(=O)[C@H](CC(=O)O)NC(=O)[C@H](Cc4ccccc4)NC(=O)[C@H](CCCNC(=N)N(C)C)NC(=O)CCC(=O)Nc4cccc5c(C(=O)NCC(=O)N6CC(F)(F)C[C@H]6C#N)ccnc45)C(=O)O)C2=O)cc1)CC3] | | |
| 42 | 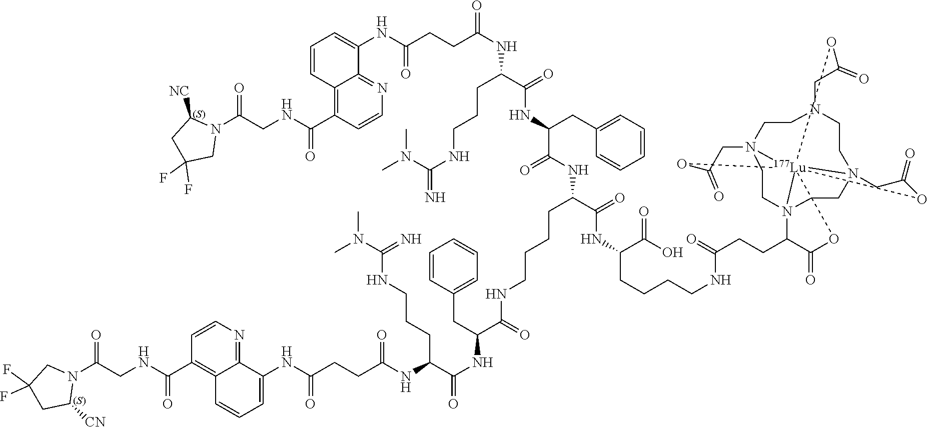 | | |
| 43 | 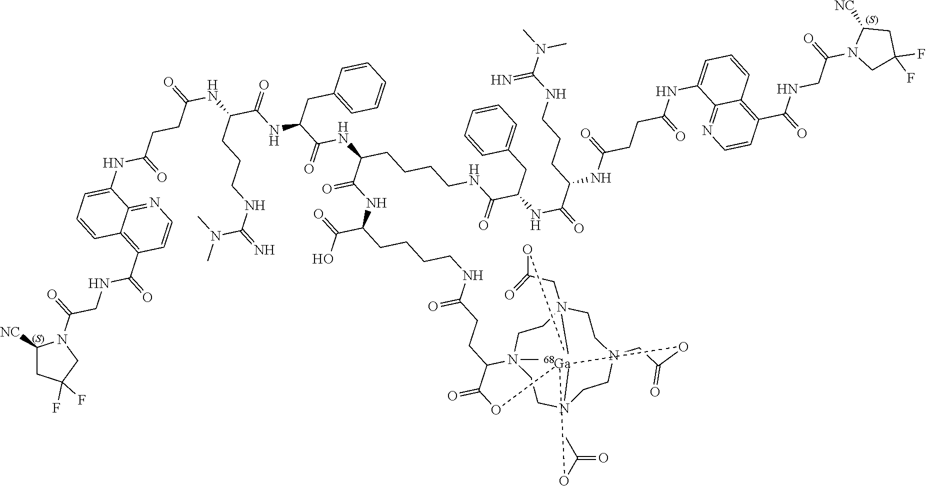 | | |
| 44 | 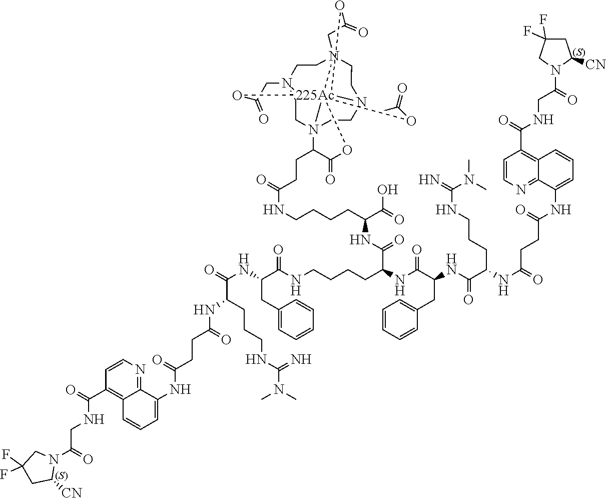 | | |
| 45 | 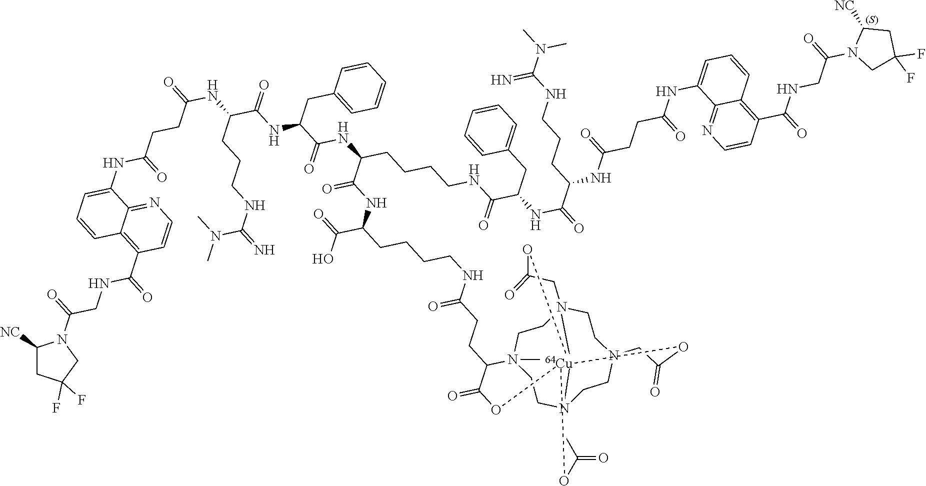 | | |
| 46 | 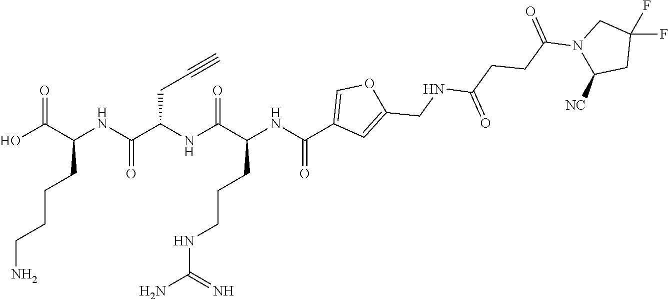 [SMILES: C#CC[C@H](NC(=O)[C@H](CCCNC(=N)N)NC(=O)c1coc(CNC(=O)CCC(=O)N2CC(F)(F)C[C@H]2C#N)c1)C(=O)N[C@@H](CCCCN)C(=O)O] | | |
| 47 | 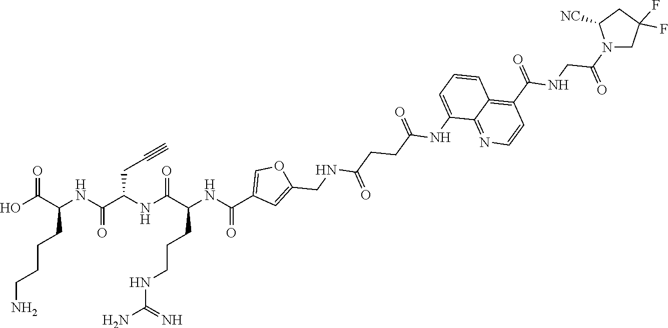 [SMILES: C#CC[C@H](NC(=O)[C@H](CCCNC(=N)N)NC(=O)c1coc(CNC(=O)CCC(=O)Nc2cccc3c(C(=O)NCC(=O)N4CC(F)(F)C[C@H]4C#N)ccnc23)c1)C(=O)N[C@@H](CCCCN)C(=O)O] | | |

In some embodiments, the binders according to the invention further may have an increased tumour-to-liver, tumour-to-kidney, tumor-to-spleen, and/or tumour-to-intestine uptake ratio; a more potent anti-tumour effect (e.g., measured by mean tumour volume increase), and/or lower toxicity (e.g., as assessed by the evaluation of changes (%) in body weight). In particular, binders according to the invention surprisingly can exhibit a very high, specific uptake in FAP-expressing tumours in combination with low uptake in normal organs. That is, the binders can provide advantageous therapeutic index in terms of tumour to non-tumour (T/NT) ratio when administered in vivo.

Further, the binders according to the invention further can have a high or improved affinity for human and murine fibroblast activation protein and/or cross-reactivity to the murine antigen. The binders according to the invention preferably attain FAP-specific cellular binding; FAP-selective accumulation on the cell membrane; FAP-selective accumulation inside the cytosol. The binders according to the invention can further preferably, rapidly and homogeneously localize at the tumour site in vivo with a high tumour-to-organs selectivity, in particular for melanoma and/or renal cell carcinoma.

Further, the binders according to the present invention can have particularly favourable metabolic stability, e.g., with a serum half-life of more than 48 hours (in human or mouse serum).

Preferred embodiments of the compounds of the present invention are shown in the appended claims, and in Tables 2 and 4.

### Moiety A

Without wishing to be bound by any theory, it is contemplated that these surprising technical effects are associated with the particular structure of the small binding moieties A. That is, an improvement for compounds comprising moiety A over a corresponding compound not having such moiety is expected to be observed.

It has been previously shown that the higher target protein affinity of a compound may result in longer tumour residence in vivo (Wichert et al., Nature Chemistry 7, 241-249 (2015)). The compounds of the present invention have an increased affinity, slower dissociation rate with respect to FAP as compared to prior art compounds, and therefore are also considered to as having a prolonged residence at the disease site at a therapeutically or diagnostically relevant level, preferably beyond 1 h, more preferably beyond 6 h post injection. Preferably, the highest enrichment is achieved after 5 min, 10 min, 20 min, 30 min, 45 min, 1 h, 2 h, 3 h, 4 h, 5 h or 6 h; and/or enrichment in the disease site is maintained at a therapeutically or diagnostically relevant level, over a period of or at least for 5 min, 10 min, 20 min, 30 min, 45 min, 1 h, 2 h, 3 h, 4 h, 5 h or 6 h, more preferably beyond 6 h post injection.

### Moiety B

Moiety B is a covalent bond or a moiety comprising a chain of atoms that covalently attaches A to the payload C, e.g., through one or more covalent bond(s). The moiety B may be cleavable or non-cleavable, multifunctional moiety which can be used to link one or more payload and/or binder moieties to form the targeted conjugate of the invention.

Specifically, moiety B is a multifunctional moiety linking one or more moieties C and/or moieties A. B can be a single bond, or an optionally substituted C₁₋₅₀ aliphatic group, in which optionally one or more carbon atoms can be replaced by a heteroatom, a C₃₋₁₂ carbocyclic or a C₁₋₁₂ heterocyclic group, and which can be saturated optionally contain one or more double or triple bonds. The structure of the compound comprises 2 moieties A per molecule. The structure of the compound may comprise more than one moieties C, preferably 2, 3, 4, 5, 6, 7, 8, 9 or 10 moieties C per molecule. Preferably, the structure of the compound comprises 2 moieties A and 1 moiety C, or 2 moieties B and 1 moiety C per molecule.

When cleavable linker units are present within moiety B, release mechanisms can be identical to those specific to antibodies linked to cytotoxic payloads. Indeed, the nature of the binding moieties is independent in that respect. Therefore, there is envisaged pH-dependent [Leamon, C. P. et al (2006) Bioconjugate Chem., 17, 1226; Casi, G. et al (2012) J. Am. Chem. Soc., 134, 5887], reductive [Bernardes, G. J. et al (2012) Angew. Chem. Int. Ed. Engl., 51. 941; Yang, J. et al (2006) Proc. Natl. Acad. Sci. USA, 103, 13872] and enzymatic release [Doronina S. O. et al (2008) Bioconjugate Chem, 19, 1960; Sutherland, M. S. K. (2006) J. Biol. Chem, 281, 10540]. In a specific setting, when functional groups are present on either the binding moiety or payloads (e.g. thiols, alcohols) a linkerless connection can be established thus releasing intact payloads, which simplifies substantially pharmacokinetic analysis.

Moiety B can comprise or consist of a unit shown in Table 3 below wherein the substituents R and Rⁿ shown in the formulae may suitably be independently selected from H, halogen, substituted or unsubstituted (hetero)alkyl, (hetero)alkenyl, (hetero)alkynyl, (hetero)aryl, (hetero)arylalkyl, (hetero)cycloalkyl, (hetero)cycloalkylaryl, heterocyclylalkyl, a peptide, an oligosaccharide or a steroid group. Preferably, each of R, R₁, R₂ and R₃ is independently selected from H, OH, SH, NH₂, halogen, cyano, carboxy, alkyl, cycloalkyl, aryl and heteroaryl, each of which is substituted or unsubstituted. Suitably R and Rⁿ are independently selected from H, or C1-C7 alkyl or heteroalkyl. More suitably, R and Rⁿ are independently selected from H, methyl or ethyl.

TABLE 3

| Linker | Structure | Release mechanism |
|---|---|---|
| Amide | 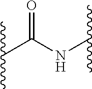 [SMILES: *NC(*)=O] | Proteolysis |
| | 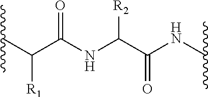 | |
| Ester | 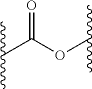 [SMILES: *OC(*)=O] | Hydrolysis |
| Carbamate | 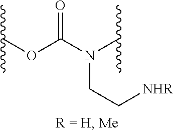 | Hydrolysis |
| Hydrazone | 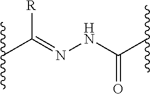 | Hydrolysis |
| Thiazolidine | 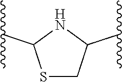 [SMILES: *C1CSC(*)N1] | Hydrolysis |
| Methylene alkoxy carbamate | 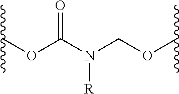 | Hydrolysis |
| Disulfide | 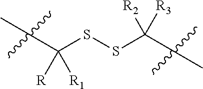 | Reduction |

Moiety B, unit(s) B_{L} and/or unit(s) B_{S} may suitably comprise as a cleavable bond a disulfide linkage since these linkages are stable to hydrolysis, while giving suitable drug release kinetics at the target in vivo, and can provide traceless cleavage of drug moieties including a thiol group.

Moiety B, unit(s) B_{L} and/or unit(s) B_{S} may be polar or charged in order to improve water solubility of the conjugate. For example, the linker may comprise from about 1 to about 20, suitably from about 2 to about 10, residues of one or more known water-soluble oligomers such as peptides, oligosaccharides, glycosaminoglycans, polyacrylic acid or salts thereof, polyethylene glycol, polyhydroxyethyl (meth) acrylates, polysulfonates, etc. Suitably, the linker may comprise a polar or charged peptide moiety comprising e.g. from 2 to 10 amino acid residues. Amino acids may refer to any natural or non-natural amino acid. The peptide linker suitably includes a free thiol group, preferably a N-terminal cysteine, for forming the said cleavable disulfide linkage with a thiol group on the drug moiety. Any peptide containing L- or D-aminoacids can be suitable; particularly suitable peptide linkers of this type are Asp-Arg-Asp-Cys and/or Asp-Lys-Asp-Cys.

In these and other embodiments, moiety B, unit(s) B_{L} and/or unit(s) B_{S} may comprise a cleavable or non-cleavable peptide unit that is specifically tailored so that it will be selectively enzymatically cleaved from the drug moiety by one or more proteases on the cell surface or the extracellular regions of the target tissue. The amino acid residue chain length of the peptide unit suitably ranges from that of a single amino acid to about eight amino acid residues. Numerous specific cleavable peptide sequences suitable for use in the present invention can be designed and optimized in their selectivity for enzymatic cleavage by a particular tumour-associated enzyme e.g. a protease. Cleavable peptides for use in the present invention include those which are optimized toward the proteases MMP-1, 2 or 3, or cathepsin B, C or D. Especially suitable are peptides cleavable by Cathepsin B. Cathepsin B is a ubiquitous cysteine protease. It is an intracellular enzyme, except in pathological conditions, such as metastatic tumours or rheumatoid arthritis. An example for a peptide cleavable by Cathepsin B is containing the sequence Val-Cit.

In any of the above embodiments, the moiety B and in particular, unit(s) B_{L} suitably further comprise(s) self-immolative moiety can or cannot be present after the linker. The self-immolative linkers are also known as electronic cascade linkers. These linkers undergo elimination and fragmentation upon enzymatic cleavage of the peptide to release the drug in active, preferably free form. The conjugate is stable extracellularly in the absence of an enzyme capable of cleaving the linker. However, upon exposure to a suitable enzyme, the linker is cleaved initiating a spontaneous self-immolative reaction resulting in the cleavage of the bond covalently linking the self-immolative moiety to the drug, to thereby effect release of the drug in its underivatized or pharmacologically active form. In these embodiments, the self-immolative linker is coupled to the binding moiety through an enzymatically cleavable peptide sequence that provides a substrate for an enzyme to cleave the amide bond to initiate the self-immolative reaction. Suitably, the drug moiety is connected to the self-immolative moiety of the linker via a chemically reactive functional group pending from the drug such as a primary or secondary amine, hydroxyl, sulfhydryl or carboxyl group.

Examples of self-immolative linkers are PABC or PAB (para-aminobenzyloxycarbonyl), attaching the drug moiety to the binding moiety in the conjugate (Carl et al (1981) J. Med. Chem. 24: 479-480; Chakravarty et al (1983) J. Med. Chem. 26: 638-644). The amide bond linking the carboxy terminus of a peptide unit and the para-aminobenzyl of PAB may be a substrate and cleavable by certain proteases. The aromatic amine becomes electron-donating and initiates an electronic cascade that leads to the expulsion of the leaving group, which releases the free drug after elimination of carbon dioxide (de Groot, et al (2001) Journal of Organic Chemistry 66 (26): 8815-8830). Further self-immolating linkers are described in WO2005/082023.

In yet other embodiments, the linker comprises a glucuronyl group that is cleavable by glucoronidase present on the cell surface or the extracellular region of the target tissue. It has been shown that lysosomal beta-glucuronidase is liberated extracellularly in high local concentrations in necrotic areas in human cancers, and that this provides a route to targeted chemotherapy (Bosslet, K. et al. Cancer Res. 58, 1195-1201 (1998)).

In any of the above embodiments, the moiety B suitably further comprises a spacer unit. A spacer unit can be the unit B_{S}, which may be linked to the binding moiety A, for example via an amide, amine or thioether bond. The spacer unit is of a length that enables e.g. the cleavable peptide sequence to be contacted by the cleaving enzyme (e. g. cathepsin B) and suitably also the hydrolysis of the amide bond coupling the cleavable peptide to the self-immolative moiety X. Spacer units may for example comprise a divalent radical such as alkylene, arylene, a heteroarylene, repeating units of alkyloxy (e.g. polyethylenoxy, PEG, polymethyleneoxy) and alkylamino (e.g. polyethyleneamino), or diacid ester and amides including succinate, succinamide, diglycolate, malonate, and caproamide.

In any of the embodiments described therein, * represents a point of attachment to moiety A or a point of attachment for which the shortest path to moiety A comprises less atoms than that for •, as the case may be; and • represents a point of attachment a point of attachment to moiety C or a point of attachment to moiety C for which the shortest path to moiety C comprises less atoms than that for *, as the case may be. The same applies also for cases where a reactive moiety L is present rather than payload moiety C. The following notations and all have the meaning of a point of attachment of a certain group or atom (e.g., R) to a further moiety:

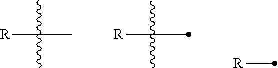

As used herein, and unless specified otherwise the groups and fragments described herein may be combined in either orientation, but it is preferred that they are combined in the orientation as drawn herein, reading from left to right, for example:
Fragment (a):

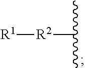

fragment (b):

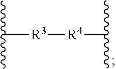

preferred combination of fragments (a)+(b):

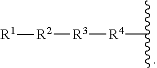

If the structure of relevance is a peptide mono- or oligomer, each * represents a point of attachment for which the shortest path to moiety A comprises less atoms than that for •; and each • represents a point of attachment for which the shortest path to moiety C comprises less atoms than that for *, with the proviso that when n is >1 and a respective point of attachment is indicated on any one of R^{a}, R^{b} and R^{c}, then it can be independently present in one or more of the peptide monomeric units, preferably in one peptide monomeric unit most distant from the other point of attachment indicated in the respective structure.

In any of the embodiments described herein, the terms “peptide”, “dipeptide”, “tripeptide”, “tetrapeptide” etc. refer to peptide mono- or oligomers having a backbone formed by proteinogenic and/or a non-proteinogenic amino acids. As used herein, the terms “aminoacyl” or “aminoacid” generally refer to any proteinogenic or a non-proteinogenic amino acid. Preferably, in any of the embodiments disclosed therein, the side-chain residues of a proteinogenic or a non-proteinogenic amino acid are represented by any of R^{a}, R^{b} and R^{c}, each of which is selected from the following list:

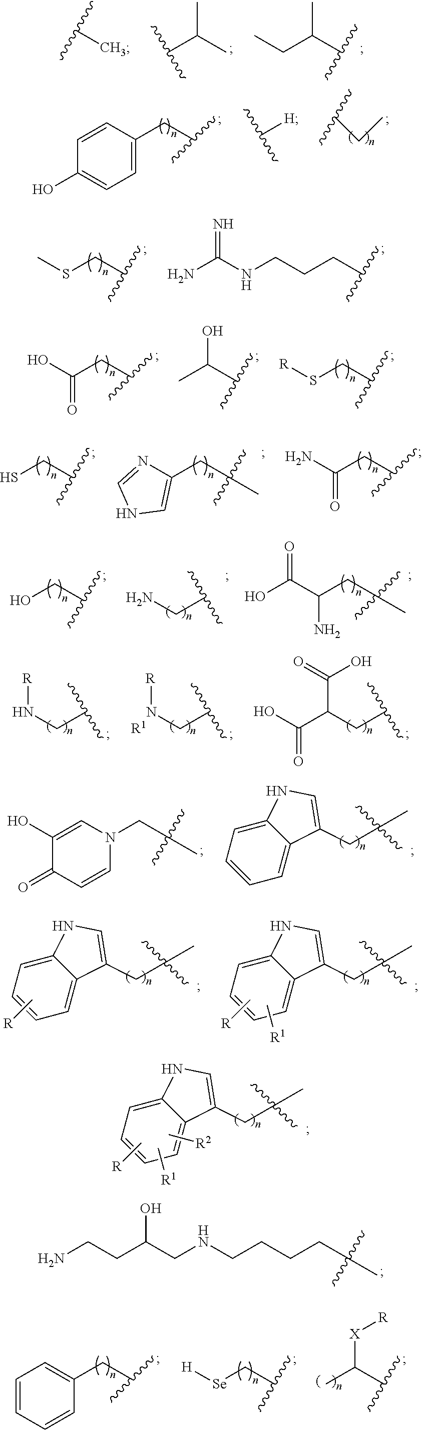

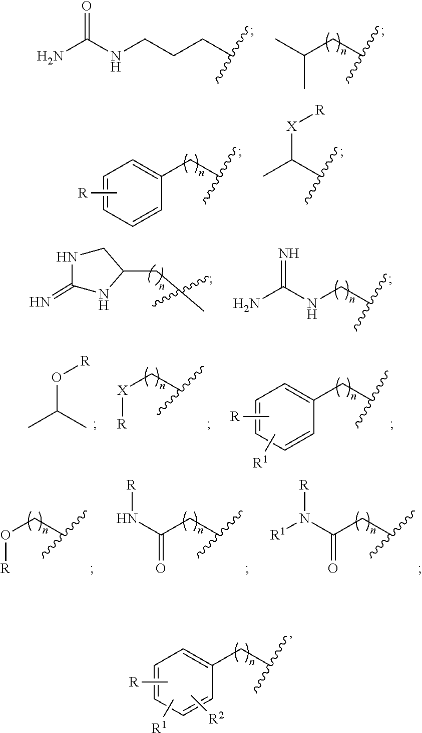

wherein each of R, R¹, R² and R³ is independently selected from H, OH, SH, NH₂, halogen, cyano, carboxy, alkyl, cycloalkyl, aryl and heteroaryl, each of which is substituted or unsubstituted; each X is independently selected from NH, NR, S, O and CH₂, preferably NH; and
each n and m is independently an integer preferably selected from 0, 1, 2, 3, 4, 5, 6, 7, 8, 9, 10, 11, 12, 13, 14, 15, 16, 17, 18, 19 and 20.

Preferably, in any of the embodiments disclosed herein, side-chain residues of a proteinogenic or a non-proteinogenic amino acid are represented by any of R^{a}, R^{b}, R^{c}, R^{d} and R^{e},
each of which may be part of a 3-, 4-, 5-, 6- or 7-membered ring. For instance, the side chain alpha, beta and/or gamma position of said proteinogenic or non-proteinogenic amino acid can be part of a cyclic structure selected from an azetidine ring, pyrrolidine ring and a piperidine ring, such as in the following aminoacids (proline and hydroxyproline):

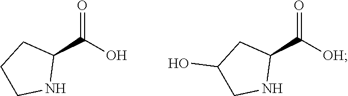 [SMILES: O=C(O)[C@@H]1CCCN1 ; O=C(O)[C@@H]1CC(O)CN1]

each of which may independently be part of an unsaturated structure (i.e. wherein the H atom geminal to the respective group R^{a}, R^{b} and R^{c} is absent), e.g.:

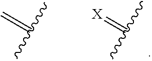

As used herein, the following notation of peptide sequences refers to a sequence from N to C terminus, and attachment of group through a horizontal bond (here: moiety C) means covalent attachment to the peptide backbone via amide bond to the respective terminal amino acid (here: AA3):

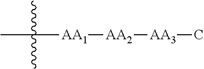

As used herein, the following notation of peptide sequences refers to a sequence from N to C terminus, and attachment of group through a vertical bond (here: moiety C) means covalent attachment via the sidechain of the respective amino acid (here: AA3):

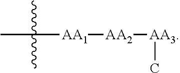

Further preferable non-proteinogenic amino acids can be selected from the following list:

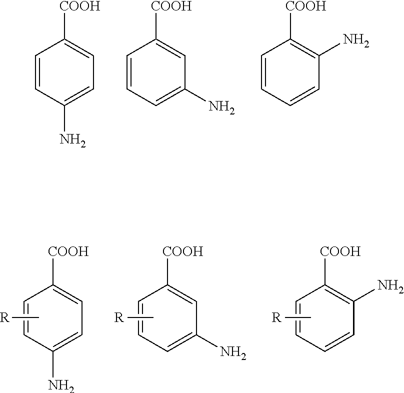

Particularly preferred embodiments for the moiety B as well as the compound according to the present invention are shown in the appended claims.

### Moiety C

Moiety C in the present invention represents a payload, which can be generally any atom (including H), molecule or particle. Preferably, moiety C is not a hydrogen atom.

The payload may be a chelator for radiolabelling. Suitably the radionuclide is not released. Chelators are well known to those skilled in the art, and for example, include chelators such as sulfur colloid, diethylenetriaminepentaacetic acid (DTPA), ethylenediaminetetraacetic acid (EDTA), 1,4,7,10-tetraazacyclododecane-N,N′,N″,N″′-tetraacetic acid (DOTA), 1,4,7,10-tetraazacyclododececane,N-(glutaric acid)-N′,N″,N″′-triacetic acid (DOTAGA), 1,4,7-triazacyclononane-N,N′,N″-triacetic acid (NOTA), 1,4,8,11-tetraazacyclotetradecane-N,N′,N″,N″′-tetraacetic acid (TETA), or any of the preferred chelator structures recited in the appended claims.

The payload may be a radioactive group comprising or consisting of radioisotope including isotopes such as ²²³Ra, ⁸⁹Sr, ^{94m}Tc, ^{99m}Tc, ¹⁸⁶Re, ¹⁸⁸Re, ²⁰³Pb, ⁶⁷Ga, ⁶⁸Ga, ⁴⁷Sc, ¹¹¹In, ⁹⁷Ru, ⁶²Cu, ⁶⁴Cu, ⁸⁶Y, ⁸⁸Y, ⁹⁰Y, ¹²¹Sn, ¹⁶¹Tb, ¹⁵³Sm, ¹⁶⁶Ho, ¹⁰⁵Rh, ¹⁷⁷Lu, ¹²³I, ¹²⁴I, ¹²⁵I, ¹³¹I, ¹⁸F, ²¹¹At, ²²⁵Ac, ⁸⁹Sr, ²²⁵Ac, ^{117m}Sn and ¹⁶⁹E. Preferably, positron emitters, such as ¹⁸F and ¹²⁴I, or gamma emitters, such as ^{99m}Tc, ¹¹¹In and ¹²³I, are used for diagnostic applications (e.g. for PET), while beta-emitters, such as ⁸⁹Sr, ¹³¹I, and ¹⁷⁷Lu, are preferably used for therapeutic applications. Alpha-emitters, such as ²¹¹At, ²²⁵Ac and ²²³Ra may also be used for therapy. In one preferred embodiment the radioisotope is ⁸⁹Sr or ²²³Ra. In a further preferred embodiment the radioisotope is ⁶⁸Ga.

The payload may be a chelate of a radioactive isotope, preferably of an isotope listed under above, with a chelating agent, preferably a chelating agent listed above or any of the preferred chelator structures recited in the appended claim 14 (a); or a group selected from the structures listed in claim 14 (c).

The payload may be a fluorophore group, preferably selected from a xanthene dye, acridine dye, oxazine dye, cyanine dye, styryl dye, coumarine dye, porphine dye, fluorescent metal-ligand-complex, fluorescent protein, nanocrystals, perylene dye, boron-dipyrromethene dye and phtalocyanine dye, more preferably selected from the structures listed in claim 14 (d).

The payload may be a cytotoxic and/or cytostatic agent. Such agents can inhibit or prevent the function of cells and/or cause destruction of cells. Examples of cytotoxic agents include radioactive isotopes, chemotherapeutic agents, and toxins such as small molecule toxins or enzymatically active toxins of bacterial, fungal, plant or animal origin, including synthetic analogues and derivatives thereof. The cytotoxic agent may be selected from the group consisting of an auristatin, a DNA minor groove binding agent, a DNA minor groove alkylating agent, an enediyne, a lexitropsin, a duocarmycin, a taxane, a puromycin, a dolastatin, a maytansinoid and a vinca alkaloid or a combination of two or more thereof. Preferred cytotoxic and/or cytostatic payload moieties are listed in claim 14 (e).

In one embodiment the payload is a chemotherapeutic agent selected from the group consisting of a topoisomerase inhibitor, an alkylating agent (e.g., nitrogen mustards; ethylenimes; alkylsulfonates; triazenes; piperazines; and nitrosureas), an antimetabolite (e.g., mercaptopurine, thioguanine, 5-fluorouracil), an antibiotics (e.g., anthracyclines, dactinomycin, bleomycin, adriamycin, mithramycin. dactinomycin) a mitotic disrupter (e.g., plant alkaloids—such as vincristine and/or microtubule antagonists—such as paclitaxel), a DNA methylating agent, a DNA intercalating agent (e.g., carboplatin and/or cisplatin, daunomycin and/or doxorubicin and/or bleomycin and/or thalidomide), a DNA synthesis inhibitor, a DNA-RNA transcription regulator, an enzyme inhibitor, a gene regulator, a hormone response modifier, a hypoxia-selective cytotoxin (e.g., tirapazamine), an epidermal growth factor inhibitor, an anti-vascular agent (e.g., xanthenone 5,6-dimethylxanthenone-4-acetic acid), a radiation-activated prodrug (e.g., nitroarylmethyl quaternary (NMQ) salts) or a bioreductive drug or a combination of two or more thereof. In some embodiments, the payload (i.e., moiety C) is not derived from an anthracycline, preferably not derived from PNU 159682.

The chemotherapeutic agent may selected from the group consisting of Erlotinib (TARCEVA®), Bortezomib (VELCADE®), Fulvestrant (FASLODEX®), Sutent (SU11248), Letrozole (FEMARA®), Imatinib mesylate (GLEEVEC®), PTK787/ZK 222584, Oxaliplatin (Eloxatin®), 5-FU (5-fluorouracil), Leucovorin, Rapamycin (Sirolimus, RAPAMUNE®), Lapatinib (GSK572016), Lonafarnib (SCH 66336), Sorafenib (BAY43-9006), and Gefitinib (IRESSA®), AG1478, AG1571 (SU 5271; Sugen) or a combination of two or more thereof.

The chemotherapeutic agent may be an alkylating agent—such as thiotepa, CYTOXAN® and/or cyclosphosphamide; an alkyl sulfonate—such as busulfan, improsulfan and/or piposulfan; an aziridine—such as benzodopa, carboquone, meturedopa and/or uredopa; ethylenimines and/or methylamelamines—such as altretamine, triethylenemelamine, triethylenepbosphoramide, triethylenethiophosphoramide and/or trimethylomelamine; acetogenin—such as bullatacin and/or bullatacinone; camptothecin; bryostatin; callystatin; cryptophycins; dolastatin; duocarmycin; eleutherobin; pancratistatin; sarcodictyin; spongistatin; nitrogen mustards—such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide and/or uracil mustard; nitrosureas—such as carmustine, chlorozotocin, fotemustine, lomustine, nimustine, and/or ranimnustine; dynemicin; bisphosphonates—such as clodronate; an esperamicin; a neocarzinostatin chromophore; aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN®. doxorubicin—such as morpholino-doxorubicin, cyanomorpholino-doxorubicin, 2-pyrrolino-doxorubicin and/or deoxydoxorubicin, epirubicin, esorubicin, idarubicin, marcellomycin, mitomycins—such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites—such as methotrexate and 5-fluorouracil (5-FU); folic acid analogues—such as denopterin, methotrexate, pteropterin, trimetrexate; purine analogues—such as fludarabine, 6-mercaptopurine, thiamiprine, thioguanine; pyrimidine analogues—such as ancitabine, azacitidine, 6-azauridine, carmofur, cytarabine, dideoxyuridine, doxifluridine, enocitabine, floxuridine; androgens—such as calusterone, dromostanolone propionate, epitiostanol, mepitiostane, testolactone; anti-adrenals—such as aminoglutethimide, mitotane, trilostane; folic acid replenisher—such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; macrocyclic depsipeptides such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofiran; spirogermanium; tenuazonic acid; triaziquone; 2,2′,2″-trichlorotriethylamine; trichothecenes—such as verracurin A, roridin A and/or anguidine; urethan; vindesine; dacarbazine; mannomustine; mitobronitol; mitolactol; pipobroman; gacytosine; arabinoside; cyclophosphamide; thiotepa; taxoids—such as TAXOL®. paclitaxel, abraxane, and/or TAXOTERE®, doxetaxel; chloranbucil; GEMZAR®. gemcitabine; 6-thioguanine; mercaptopurine; methotrexate; platinum analogues—such as cisplatin and carboplatin; vinblastine; platinum; etoposide; ifosfamide; mitoxantrone; vincristine; NAVELBINE®, vinorelbine; novantrone; teniposide; edatrexate; daunomycin; aminopterin; xeloda; ibandronate; topoisomerase inhibitor RFS 2000; difluoromethylomithine (DMFO); retinoids—such as retinoic acid; capecitabine; and pharmaceutically acceptable salts, acids, derivatives or combinations of two or more of any of the above.

The payload may be a tubulin disruptor including but are not limited to: taxanes—such as paclitaxel and docetaxel, vinca alkaloids, discodermolide, epothilones A and B, desoxyepothilone, cryptophycins, curacin A, combretastatin A-4-phosphate, BMS 247550, BMS 184476, BMS 188791; LEP, RPR 109881A, EPO 906, TXD 258, ZD 6126, vinflunine, LU 103793, dolastatin 10, E7010, T138067 and T900607, colchicine, phenstatin, chalcones, indanocine, T138067, oncocidin, vincristine, vinblastine, vinorelbine, vinflunine, halichondrin B, isohomohalichondrin B, ER-86526, pironetin, spongistatin 1, spiket P, cryptophycin 1, LU103793 (cematodin or cemadotin), rhizoxin, sarcodictyin, eleutherobin, laulilamide, VP-16 and D-24851 and pharmaceutically acceptable salts, acids, derivatives or combinations of two or more of any of the above.

The payload may be a DNA intercalator including but are not limited to: acridines, actinomycins, anthracyclines, benzothiopyranoindazoles, pixantrone, crisnatol, brostallicin, CI-958, doxorubicin (adriamycin), actinomycin D, daunorubicin (daunomycin), bleomycin, idarubicin, mitoxantrone, cyclophosphamide, melphalan, mitomycin C, bizelesin, etoposide, mitoxantrone, SN-38, carboplatin, cis-platin, actinomycin D, amsacrine, DACA, pyrazoloacridine, irinotecan and topotecan and pharmaceutically acceptable salts, acids, derivatives or combinations of two or more of any of the above.

The payload may be an anti-hormonal agent that acts to regulate or inhibit hormone action on tumours—such as anti-estrogens and selective estrogen receptor modulators, including, but not limited to, tamoxifen, raloxifene, droloxifene, 4-hydroxytamoxifen, trioxifene, keoxifene, LY117018, onapristone, and/or fareston toremifene and pharmaceutically acceptable salts, acids, derivatives or combinations of two or more of any of the above. The payload may be an aromatase inhibitor that inhibits the enzyme aromatase, which regulates estrogen production in the adrenal glands—such as, for example, 4(5)-imidazoles, aminoglutethimide, megestrol acetate, AROMASIN®. exemestane, formestanie, fadrozole, RIVISOR®. vorozole, FEMARA®. letrozole, and ARIMIDEX® and/or anastrozole and pharmaceutically acceptable salts, acids, derivatives or combinations of two or more of any of the above.

The payload may be an anti-androgen such as flutamide, nilutamide, bicalutamide, leuprolide, goserelin and/or troxacitabine and pharmaceutically acceptable salts, acids, derivatives or combinations of two or more of any of the above.

The payload may be a protein or an antibody. Preferably, the payload is a cytokine (e.g., an interleukin such as IL2, IL10, IL12, IL15; a member of the TNF superfamily; or an interferon such as interferon gamma).

Any payload may be used in unmodified or modified form. Combinations of payloads in which some are unmodified and some are modified may be used. For example, the payload may be chemically modified. One form of chemical modification is the derivatisation of a carbonyl group—such as an aldehyde.

In a preferred embodiment, the payload moiety C is a topoisomerase inhibitor; preferably camptothecin (CPT) or a derivative thereof; more preferably derived (e.g., by replacing a hydrogen atom) from topotecan, irinotecan, silatecan, cositecan, exatecan, lurtotecan, gimatecan, belotecan, rubitecan; even more preferably exatecan; even more preferably

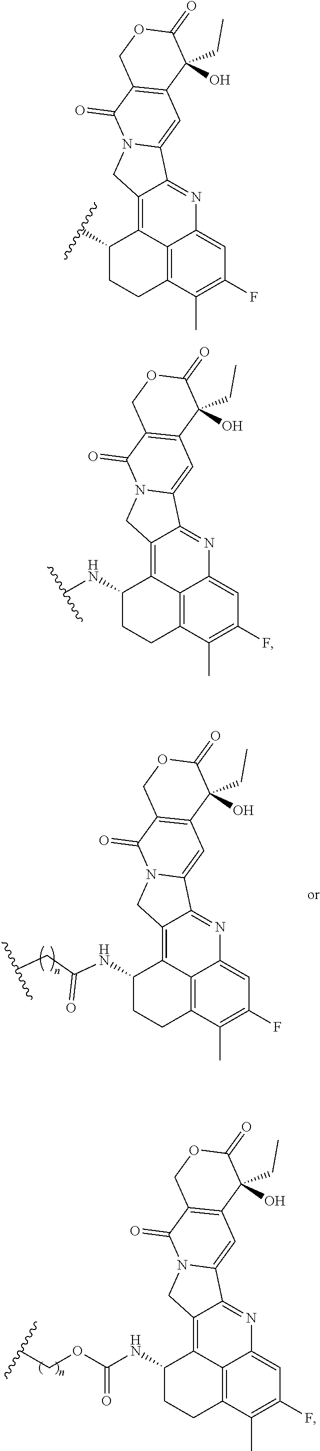

wherein each n is 0, 1, 2, 3, 4, 5 or 6; and most preferably

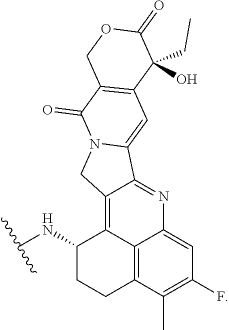 [SMILES: *N[C@H]1CCc2c(C)c(F)cc3nc4c(c1c23)Cn1c-4cc2c(c1=O)COC(=O)[C@]2(O)CC]

In a preferred embodiment, moiety C is an auristatin (i.e., having a structure derived from an auristatin compound family member) or an auristatin derivative. More preferably, moiety C has a structure according to the following formula:

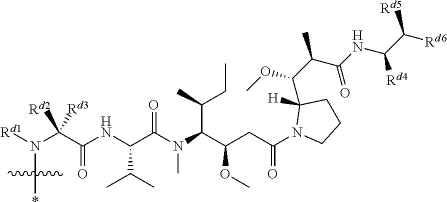

wherein:
R^{1d} is independently H or C₁-C₆ alkyl; preferably H or CH₃;
R^{2d} is independently C₁-C₆ alkyl; preferably CH₃ or iPr;
R^{3d} is independently H or C₁-C₆ alkyl; preferably H or CH₃;
R^{4d} is independently H, C₁-C₆ alkyl, COO(C₁-C₆ alkyl), CON(H or C₁-C₆ alkyl), C₃-C₁₀ aryl or C₃-C₁₀ heteroaryl; preferably H, CH₃, COOH, COOCH₃ or thiazolyl;
R^{5d} is independently H, OH, C₁-C₆ alkyl; preferably H or OH; and
R^{6d} is independently C₃-C₁₀ aryl or C₃-C₁₀ heteroaryl; preferably optionally substituted phenyl or pyridyl.

More preferably, moiety C is derived from MMAE or MMAF.

In a preferred embodiment, moiety C has a structure according to the following formula:

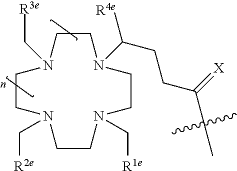

wherein:
n is 0, 1, 2, 3, 4 or 5; preferably 1;
R^{1e} is independently H, COOH, aryl-COOH or heteroaryl-COOH; preferably COOH;
R^{2e} is independently H, COOH, aryl-COOH or heteroaryl-COOH; preferably COOH;
each R^{3e} is independently H, COOH, aryl-COOH or heteroaryl-COOH; preferably COOH;
R^{4e} is independently H, COOH, aryl-COOH or heteroaryl-COOH; preferably COOH; and
X is O, NH or S; preferably 0.

In a preferred embodiment, moiety C has a structure according to the following formula:

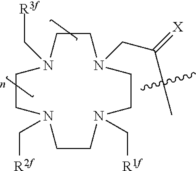

wherein:
n is 0, 1, 2, 3, 4 or 5; preferably 1
R^{1f} is independently H, COOH, aryl-COOH or heteroaryl-COOH; preferably COOH;
R^{2f} is independently H, COOH, aryl-COOH or heteroaryl-COOH; preferably COOH;
R^{3f} is independently H, COOH, aryl-COOH or heteroaryl-COOH; preferably COOH; and
X is O, NH or S; preferably O

Particularly preferred embodiments for the moiety C as well as the compound according to the present invention are shown in the appended claims.

Preferred compounds according to the present invention may be represented by:

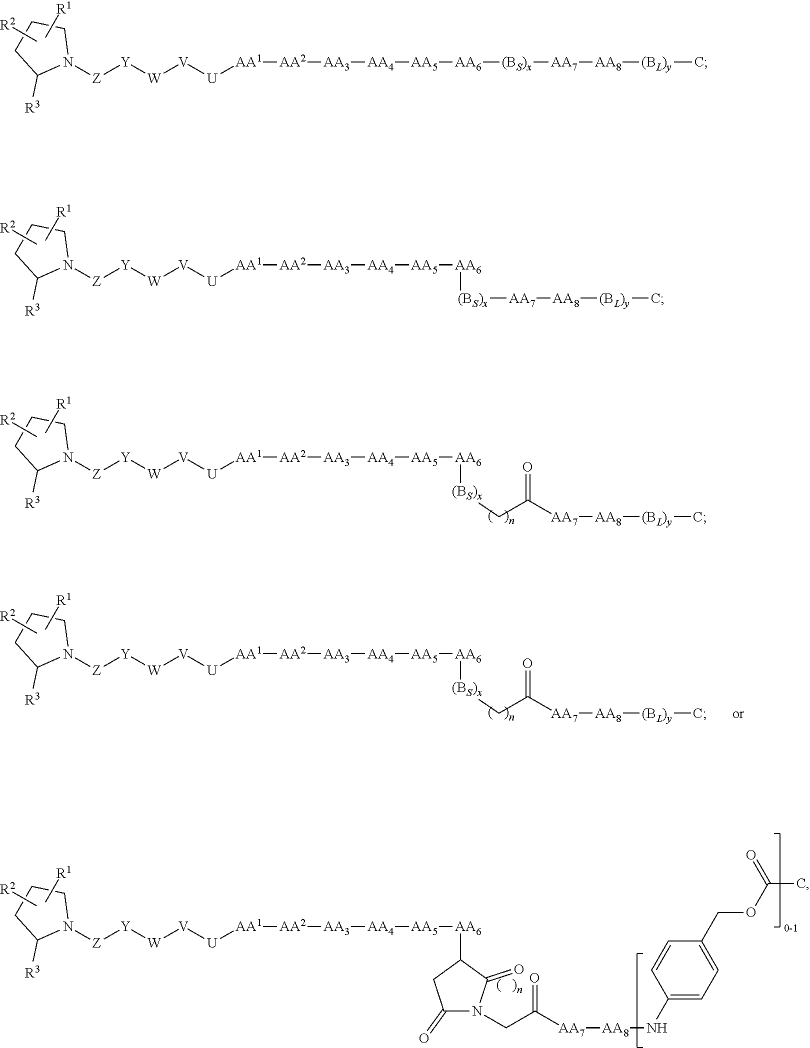

wherein B_{S}, B_{L}, x, y and n and the remaining groups are as defined elsewhere herein; more preferably

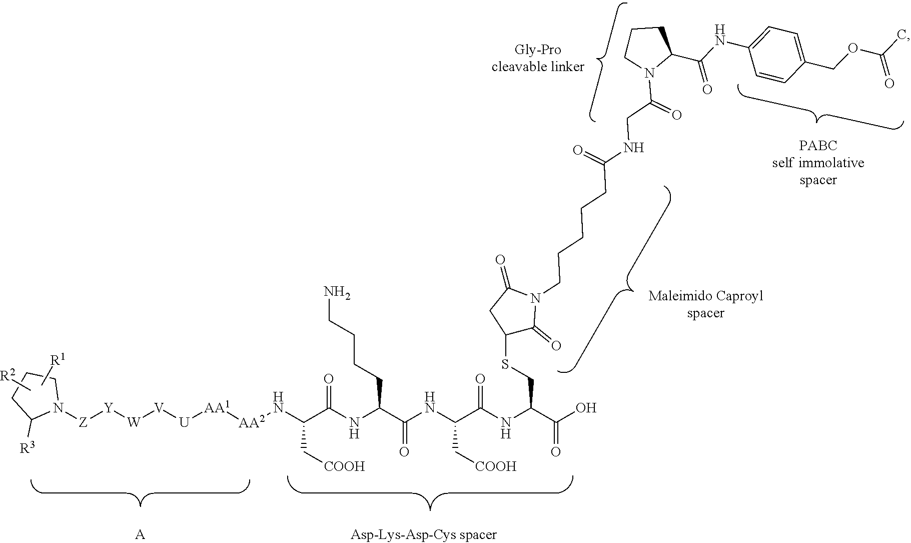

most preferably

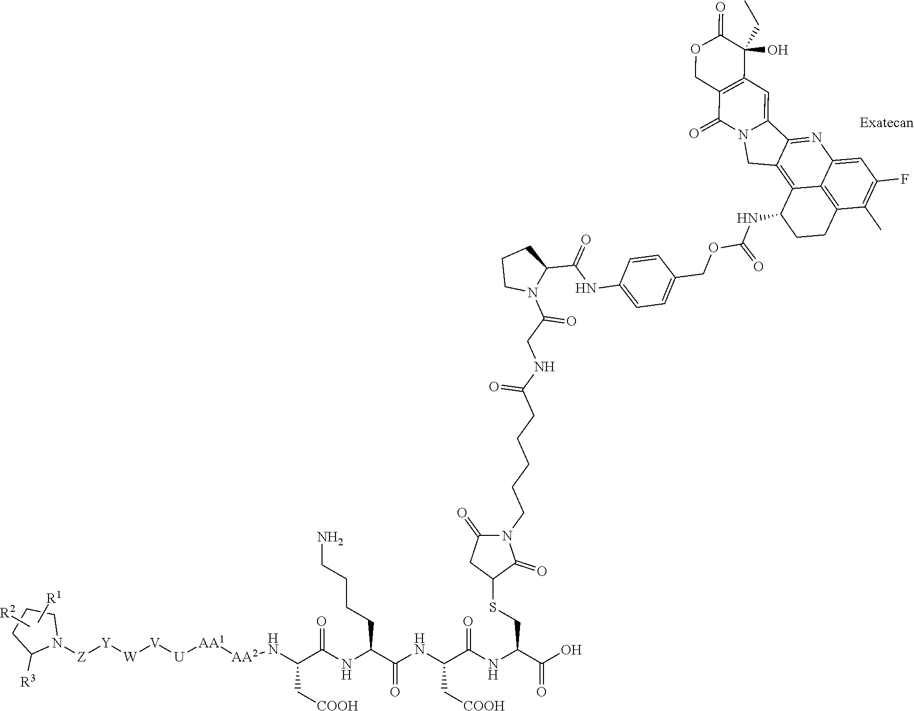

Preferred compounds according to the present invention may be represented by

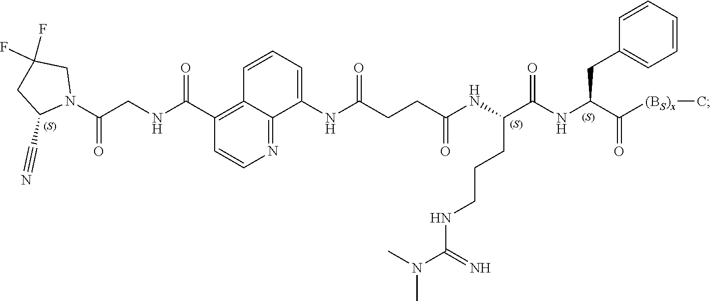

more preferably

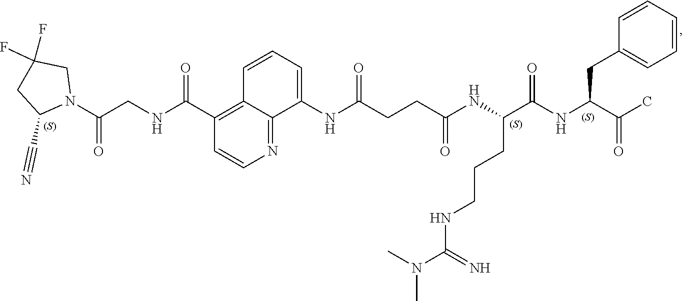

wherein:
each B_{S} is as defined elsewhere herein, and x is 0, 1, 2, 3, 4, 5, 6, 7, 8, 9, or 10; and
each C is as defined elsewhere herein, and is preferably a therapeutic moiety, more preferably a cytotoxic and/or cytostatic agent, e.g., exatecan.

Further preferred compounds according to the present invention may be represented by:

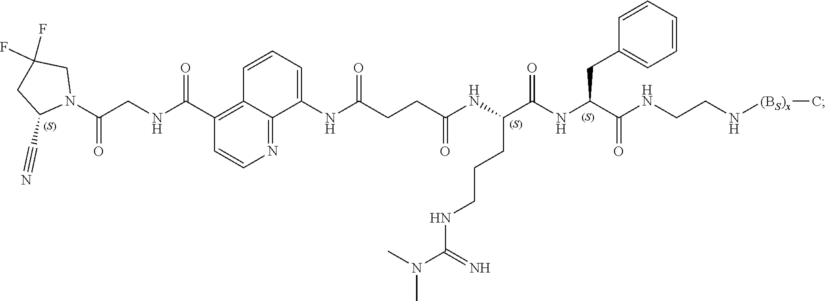

more preferably

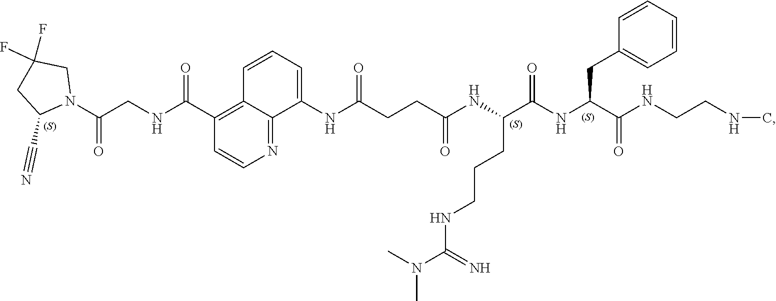

wherein:
each B_{S} is as defined elsewhere herein, and x is 0, 1, 2, 3, 4, 5, 6, 7, 8, 9, or 10; and
each C is as defined elsewhere herein, and is preferably a chelating agent group suitable for radiolabeling, a radioactive group comprising a radioisotope, or a chelate of a radioactive isotope with a chelating agent, e.g., DOTAGA.

Preferred compounds are those having a structure according to Table 2 or 4, their individual diastereoisomers, hydrates, solvates, crystal forms, individual tautomers or pharmaceutically acceptable salts thereof.

In all structures, unless otherwise specified, all groups and variables are defined as further above in the present disclosure.

Also disclosed is a pharmaceutical composition comprising the compound according to any of the preceding aspects, and a pharmaceutically acceptable excipient. Such pharmaceutical composition is also disclosed for use in: (a) a method for treatment of the human or animal body by surgery or therapy or a diagnostic method practised on the human or animal body; or (b) a method for therapy or prophylaxis of a subject suffering from or having risk for a disease or disorder; or (c) a method for guided surgery practised on a subject suffering from or having risk for a disease or disorder; or (d) a method for diagnosis of a disease or disorder, the method being practised on the human or animal body and involving a nuclear medicine imaging technique, such as Positron Emission Tomography (PET) or Single Photon Emission Computed Tomography (SPECT); or (e) a method for targeted delivery of a therapeutic or diagnostic agent to a subject suffering from or having risk for a disease or disorder, wherein in each of the preceding (b)-(e), said disease or disorder is independently selected from cancer, inflammation, atherosclerosis, fibrosis, tissue remodelling and keloid disorder, preferably wherein the cancer is selected from the group consisting of breast cancer, pancreatic cancer, small intestine cancer, colon cancer, multi-drug resistant colon cancer, rectal cancer, colorectal cancer, metastatic colorectal cancer, lung cancer, non-small cell lung cancer, head and neck cancer, ovarian cancer, hepatocellular cancer, oesophageal cancer, hypopharynx cancer, nasopharynx cancer, larynx cancer, myeloma cells, bladder cancer, cholangiocarcinoma, clear cell renal carcinoma, neuroendocrine tumour, oncogenic osteomalacia, sarcoma, CUP (carcinoma of unknown primary), thymus cancer, desmoid tumours, glioma, astrocytoma, cervix cancer and prostate cancer; preferably wherein the compound has a prolonged residence at the disease site at a therapeutically or diagnostically relevant level, preferably beyond 1 h, more preferably beyond 6 h post injection.

### Treatment

The compounds described herein may be used to treat disease. The treatment may be therapeutic and/or prophylactic treatment, with the aim being to prevent, reduce or stop an undesired physiological change or disorder. The treatment may prolong survival as compared to expected survival if not receiving treatment. The disease that is treated by the compound may be any disease that might benefit from treatment. This includes chronic and acute disorders or diseases including those pathological conditions which predispose to the disorder.

The term “cancer” and “cancerous” is used in its broadest sense as meaning the physiological condition in mammals that is typically characterized by unregulated cell growth. A tumour comprises one or more cancerous cells. When treating cancer, the therapeutically effect that is observed may be a reduction in the number of cancer cells; a reduction in tumour size; inhibition or retardation of cancer cell infiltration into peripheral organs; inhibition of tumour growth; and/or relief of one or more of the symptoms associated with the cancer.

In animal models, efficacy may be assessed by physical measurements of the tumour during the treatment, and/or by determining partial and complete remission of the cancer. For cancer therapy, efficacy can, for example, be measured by assessing the time to disease progression (TTP) and/or determining the response rate (RR).

Particularly preferred embodiments for the methods of treatment related to the present invention are shown in the appended claims.

Herein disclosed are also methods for treatment of the human or animal body, e.g., by surgery or therapy, or diagnostic method practised on the human or animal body, the methods involving a step of administering a therapeutically or diagnostically effective amount of a compound or a pharmaceutical composition as described herein to a subject in need thereof. More specifically, herein disclosed are methods for treatment, e.g., by therapy or prophylaxis, of a subject suffering from or having risk for a disease or disorder; or by guided surgery practised on a subject suffering from or having risk for a disease or disorder; method for diagnosis of a disease or disorder, e.g., diagnostic method practised on the human or animal body and/or involving a nuclear medicine imaging technique, such as Positron Emission Tomography (PET) or Single Photon Emission Computed Tomography (SPECT); method for targeted delivery of a therapeutic or diagnostic agent to a subject suffering from or having risk for a disease or disorder. In the aforementioned methods, said disease or disorder may be independently selected from cancer, inflammation, atherosclerosis, fibrosis, tissue remodelling and keloid disorder, preferably wherein the cancer is selected from the group consisting of breast cancer, pancreatic cancer, small intestine cancer, colon cancer, multi-drug resistant colon cancer, rectal cancer, colorectal cancer, metastatic colorectal cancer, lung cancer, non-small cell lung cancer, head and neck cancer, ovarian cancer, hepatocellular cancer, oesophageal cancer, hypopharynx cancer, nasopharynx cancer, larynx cancer, myeloma cells, bladder cancer, cholangiocarcinoma, clear cell renal carcinoma, neuroendocrine tumour, oncogenic osteomalacia, sarcoma, CUP (carcinoma of unknown primary), thymus cancer, desmoid tumours, glioma, astrocytoma, cervix cancer, skin cancer, kidney cancer and prostate cancer. When used in the methods disclosed herein, the compound has a prolonged residence at the disease site at a therapeutically or diagnostically relevant level, preferably beyond 1 h, more preferably beyond 6 h post injection.

### Pharmaceutical Compositions

The compounds described herein may be in the form of pharmaceutical compositions which may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

If the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example, intravenously, intramuscularly or subcutaneously. For parenteral administration, the compositions may be best used in the form of a sterile aqueous solution which may contain other substances, for example, enough salts or monosaccharides to make the solution isotonic with blood. For buccal or sublingual administration, the compositions may be administered in the form of tablets or lozenges which can be formulated in a conventional manner.

The compound of the present invention may be administered in the form of a pharmaceutically acceptable or active salt. Pharmaceutically-acceptable salts are well known to those skilled in the art, and for example, include those mentioned by Berge et al, in J. Pharm. Sci., 66, 1-19 (1977). Salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The routes for administration (delivery) may include, but are not limited to, one or more of oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

The formulations may be packaged in unit-dose or multi-dose containers, for example sealed ampoules and vials, and may be stored in a freeze-dried (lyophilized) condition requiring only the addition of the sterile liquid carrier, for example water, for administration. Extemporaneous injection solutions and suspensions are prepared from sterile powders, granules and tablets of the kind previously described. Exemplary unit dosage formulations contain a daily dose or unit daily sub-dose, or an appropriate fraction thereof, of the active ingredient.

### Precursor Compounds

In one aspect of the invention, herein disclosed is a compound, its individual diastereoisomers, its hydrates, its solvates, its crystal forms, its individual tautomers or a salt thereof, wherein the compound (precursor compound) comprises two moieties A and a reactive moiety L capable of reacting and forming a covalent bond with a conjugation partner. Upon conjugation (i.e., reacting and forming a covalent bond), the former precursor compound is bound to the former conjugation partner, which in turn to a payload moiety C. The conjugation partner can be an atom, a molecule, a particle, a therapeutic agent and/or diagnostic agent. Preferably, the conjugation is a therapeutic agent and/or diagnostic agent, and can correspond to the payload moieties already described in detail above with respect to the conjugates according to the invention.

In all structures, unless otherwise specified, all groups and variables are as defined further above throughout the present disclosure.

### Methods for Preparing a Conjugate

In one aspect of the invention, herein disclosed is a method for preparing a conjugate comprising the step of conjugating with a precursor compound as described above with a conjugation partner. Preferably, the precursor compound is conjugated to the conjugation partner by reacting therewith to form a covalent bond. Preferably, the thus obtained conjugate is a conjugate compound as described elsewhere in the present specification.

The conjugation partner can be an atom, a molecule, a particle, a therapeutic agent and/or diagnostic agent. Preferably, the conjugation is a therapeutic agent and/or diagnostic agent, and can correspond to the payload moieties already described in detail above with respect to the conjugates according to the invention.

Preferably, the method further comprises formulating the conjugate as a pharmaceutical composition or as a diagnostic composition. The pharmaceutical or diagnostic compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of carrier, excipient or diluent can be selected with regard to the intended route of administration and standard pharmaceutical practice. The pharmaceutical or diagnostic compositions may comprise as—or in addition to—the carrier, excipient or diluent any suitable binder(s), lubricant(s), suspending agent(s), coating agent(s), solubilising agent(s). All formulation details and aspects disclosed above in the section “Pharmaceutical compositions” fully apply here too.

### General Techniques

The practice of the present invention employs, unless otherwise indicated, conventional methods of chemistry, biochemistry, molecular biology, cell biology, genetics, immunology and pharmacology, known to those of skill of the art. Such techniques are explained fully in the literature. See, e. g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

### Chemical Synthesis

The compounds described herein may be prepared by chemical synthesis techniques. It will be apparent to those skilled in the art that sensitive functional groups may need to be protected and deprotected during synthesis of a compound. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994). It is possible during some of the reactions that any stereocentres present could, under certain conditions, be epimerised, for example if a base is used in a reaction with a substrate having an optical centre comprising a base-sensitive group. It should be possible to circumvent potential problems such as this by choice of reaction sequence, conditions, reagents, protection/deprotection regimes, etc. as is well-known in the art.

### Definitions

Antibody. The term “antibody” is used in its broadest sense and covers monoclonal antibodies, polyclonal antibodies, dimers, multimers, multispecific antibodies (e.g., bispecific antibodies), veneered antibodies, antibody fragments and small immune proteins (SIPs) (see Int. J. Cancer (2002) 102, 75-85). An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e. a molecule that contains an antigen binding site that immunospecifically binds an antigen of a target of interest or part thereof. The antibodies may be of any type—such as IgG, IgE, IgM, IgD, and IgA)—any class—such as IgG1, IgG2, IgG3, IgG4, IgA1 and IgA2—or subclass thereof. The antibody may be or may be derived from murine, human, rabbit or from other species.

Antibody fragments. The term “antibody fragment” refers to a portion of a full length antibody, generally the antigen binding or variable region thereof. Examples of antibody fragments include, but are not limited to, Fab, Fab′, F(ab′)₂, and Fv fragments; diabodies; linear antibodies; single domain antibodies, including dAbs, camelid VHH antibodies and the IgNAR antibodies of cartilaginous fish. Antibodies and their fragments may be replaced by binding molecules based on alternative non-immunoglobulin scaffolds, peptide aptamers, nucleic acid aptamers, structured polypeptides comprising polypeptide loops subtended on a non-peptide backbone, natural receptors or domains thereof.

Derivative. A derivative includes the chemical modification of a compound. Examples of such modifications include the replacement of a hydrogen by a halo group, an alkyl group, an acyl group or an amino group and the like. The modification may increase or decrease one or more hydrogen bonding interactions, charge interactions, hydrophobic interactions, van der Waals interactions and/or dipole interactions.

Analog. This term encompasses any enantiomers, racemates and stereoisomers, as well as all pharmaceutically acceptable salts and hydrates of such compounds.

Unless otherwise stated, the following definitions apply to chemical terms used in connection of compounds of the invention and compositions containing such compounds.

Alkyl refers to a branched or unbranched saturated hydrocarbyl radical. Suitably, the alkyl group comprises from 1 to 100, preferably 3 to 30, carbon atoms, more preferably from 5 to 25 carbon atoms. Preferably, alkyl refers to methyl, ethyl, propyl, butyl, pentyl, or hexyl.

Alkenyl refers to a branched or unbranched hydrocarbyl radical containing one or more carbon-carbon double bonds. Suitably, the alkenyl group comprises from 2 to 30 carbon atoms, preferably from 5 to about 25 carbon atoms.

Alkynyl refers to a branched or unbranched hydrocarbyl radical containing one or more carbon-carbon triple bonds. Suitably, the alkynyl group comprises from about 3 to about 30 carbon atoms, for example from about 5 to about 25 carbon atoms.

Halogen refers to fluorine, chlorine, bromine or iodine, preferably fluorine or chlorine.

Cycloalkyl refers to an alicyclic moiety, suitably having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbornyl, bicyclo[2.2.2]octyl and the like.

Aryl refers to an aromatic carbocyclic ring system, suitably comprising 6, 7, 8, 9, 10, 11, 12, 13, 14, 15 or 16 ring carbon atoms. Aryl may be a polycyclic ring system, having two or more rings, at least one of which is aromatic. This term includes reference to groups such as phenyl, naphthyl fluorenyl, azulenyl, indenyl, anthryl and the like.

The prefix (hetero) herein signifies that one or more of the carbon atoms of the group may be substituted by nitrogen, oxygen, phosphorus, silicon or sulfur. Heteroalkyl groups include for example, alkyloxy groups and alkythio groups. Heterocycloalkyl or heteroaryl groups herein may have from 3, 4, 5, 6, 7, 8, 9, 10, 11, 12, 13, 14, 15 or 16 ring atoms, at least one of which is selected from nitrogen, oxygen, phosphorus, silicon and sulfur. In particular, a 3- to 10-membered ring or ring system and more particularly a 5- or 6-membered ring, which may be saturated or unsaturated. For example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, 1,3-Dioxo-1,3-dihydro-isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, [beta]-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, 3,4-dihydro-2H-isoquinolin-1-one, 3,4-dihydro-2H-isoquinolinyl, and the like.

“Substituted” signifies that one or more, especially up to 5, more especially 1, 2 or 3, of the hydrogen atoms in said moiety are replaced independently of each other by the corresponding number of substituents. The term “optionally substituted” as used herein includes substituted or unsubstituted. It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Preferably, the term “substituted” signifies one or more, especially up to 5, more especially 1, 2 or 3, of the hydrogen atoms in said moiety are replaced independently of each other by the corresponding number of substituents selected from OH, SH, NH₂, halogen, cyano, carboxy, alkyl, cycloalkyl, aryl and heteroaryl. Additionally, the substituents described herein may themselves be substituted by any substituent, subject to the aforementioned restriction to appropriate substitutions as recognised by the skilled person. Preferably, any of the aforementioned substituents may be further substituted by any of the aforementioned substituents, each of which may be further substituted by any of the aforementioned substituents.

Substituents may suitably include halogen atoms and halomethyl groups such as CF₃ and CCl₃; oxygen containing groups such as oxo, hydroxy, carboxy, carboxyalkyl, alkoxy, alkoyl, alkoyloxy, aryloxy, aryloyl and aryloyloxy; nitrogen containing groups such as amino, alkylamino, dialkylamino, cyano, azide and nitro; sulfur containing groups such as thiol, alkylthiol, sulfonyl and sulfoxide; heterocyclic groups which may themselves be substituted; alkyl groups, which may themselves be substituted; and aryl groups, which may themselves be substituted, such as phenyl and substituted phenyl. Alkyl includes substituted and unsubstituted benzyl.

Where two or more moieties are described as being “each independently” selected from a list of atoms or groups, this means that the moieties may be the same or different. The identity of each moiety is therefore independent of the identities of the one or more other moieties.

### EXAMPLES

### 1. Synthesis of Derivatives

### 1.1 General Remarks for Solid Phase Synthesis

Liquid-Chromatography/Mass-Spectrometry (LC/MS) spectra were recorded on an Agilent 6100 Series Single Quadrupole MS system combined with an Agilent 1200 Series LC, using an InfinityLab Poroshell 120 EC-C18 Column, 2.7 μm, 4.6×50 mm at a flow rate of 0.6 ml min⁻¹, 10% MeCN in 0.1% aq. FA to 100% MeCN in 6 min.

All the chromatograms were registered at λ=260 nm. The purity of the products was assessed by integrating the final chromatogram between 1.1-5.0 minutes. The injection peak (0.0-1.1 minutes) was excluded.

Reversed-phase high-pressure liquid chromatography (RP-HPLC) was performed on an Agilent 1200 Series RP-HPLC with PDA UV detector, using a Synergi 4 μm, Polar-RP 80 Å 10×150 mm C18 column at a flow rate of 5 ml min⁻¹ with linear gradients of solvents A and B (A=Millipore water with 0.1% TFA, B=MeCN with 0.1% TFA).

### 1.2 Solid Phase Synthesis

The synthesis of compound 2, 3, 4, 5, 6, 9, 10, 11, 12, 13, 14, 15, 16, 17, 18, 19, 34 and 37 was performed on solid phase using pre-loaded Fmoc-L-lys(Boc)-Wang resin (200-400 mesh, 0.6 mmol/g). The commercially available resin was swelled in DMF for 30 minutes and subsequently reacted as reported in this paragraph.

### 1.2.1 General Procedure of Fmoc Deprotection

The Fmoc protecting group was removed by incubating three times the resin with piperidine:DMF=1:4 solution (1×30 minutes, 2×10 minutes). After Fmoc deprotection, the resin was washed with DMF (5×2 mL). The deprotection efficiency was confirmed by LC-MS analysis.

### 1.2.2 Coupling Procedure of Fmoc-Protected Amino-Acid or Carboxylic Acid Derivative (HATU Protocol)

A solution of Fmoc-protected amino-acid (4 equivalents) or, alternatively, a solution of carboxylic acid derivative (4 equivalents), O-(7-azabenzotriazol-1-yl)-N,N,N,N-tetramethyluronium hexafluorophosphate (HATU, 4 equivalents) and DIPEA (4 equivalents) in dry DMF was added to the “free-amino” peptide resin. The reaction was carried out at room temperature and quenched after 1 hour by washing the resin several times with DMF. The coupling efficiency was confirmed by LC-MS analysis.

### 1.2.3 Coupling Procedure of Fmoc-Protected Amino-Acid or Carboxylic Acid Derivative (EDC Protocol)

A solution of Fmoc-protected amino-acid (4 equivalents) or, alternatively, a solution of carboxylic acid derivative (4 equivalents), 1-Ethyl-3-(3-dimethylaminopropyl) carbodiimide (EDC, 4 equivalents), 1-Hydroxy-7-azabenzotriazole (HOAt, 3 equivalents) and DIPEA (4 equivalents) in dry DMF was incubated for 20 minutes at room temperature. The activated solution was added to the “free-amino” peptide resin. The reaction was carried out at room temperature and quenched after 12 hours by washing the resin several times with DMF. The coupling efficiency was confirmed by LC-MS analysis.

### 1.2.4 General Procedure of LC-MS Analysis of On-Resin Derivatives

A representative sample of beads carrying the growing peptide were poured in 50 μL of trifluoroacetic acid (99%) for 20 minutes. The reaction was diluted with 200 μL of acetonitrile:mQ Millipore water=1:1, filtered and analysed by LC-MS.

### 1.2.5 General Procedure of Resin Cleavage and Purification

The resin was incubated three times for 1 hour with a solution of anhydrous trifluoroacetic acid:triisopropylsilane=97.5:2.5 (20 mL/g). The crude product was dried under reduce pressure and dissolved in mQ Millipore water:acetonitrile=1:1 mixture (1 mL) and purified by RP-HPLC with acetonitrile, 0.1% trifluoroacetic acid (buffer B): H₂O, 0.1% trifluoroacetic acid (buffer A) as an eluent (5% B for 2 minutes; from 5% to 40% B in 8 minutes, from 40% to 60% B in 3 minutes, from 60% to 100% B in 2 minutes and 100% B for 2 mins).

### 1.3 Synthetic Procedures

### 1.3.1 Preparation of Compound 2

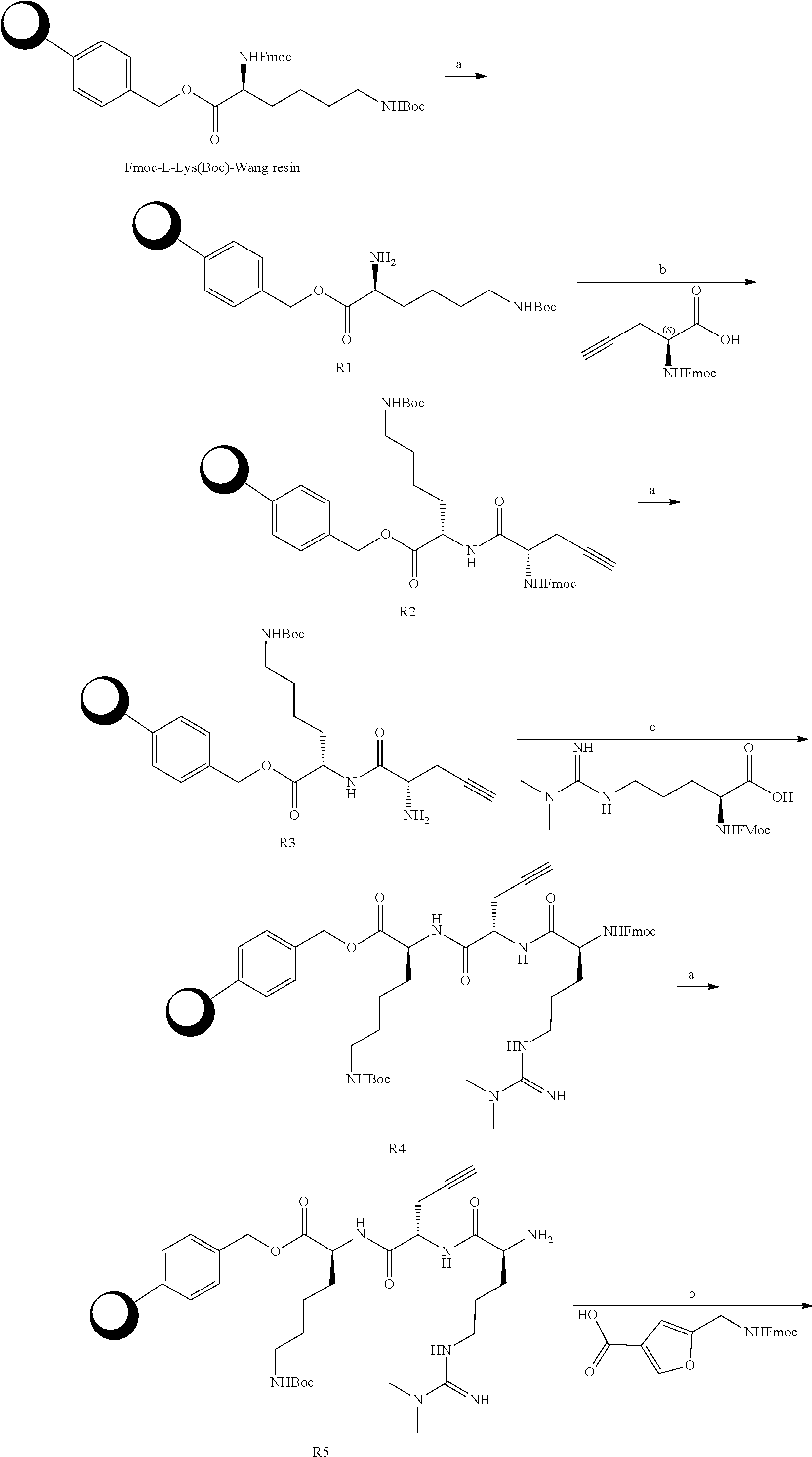

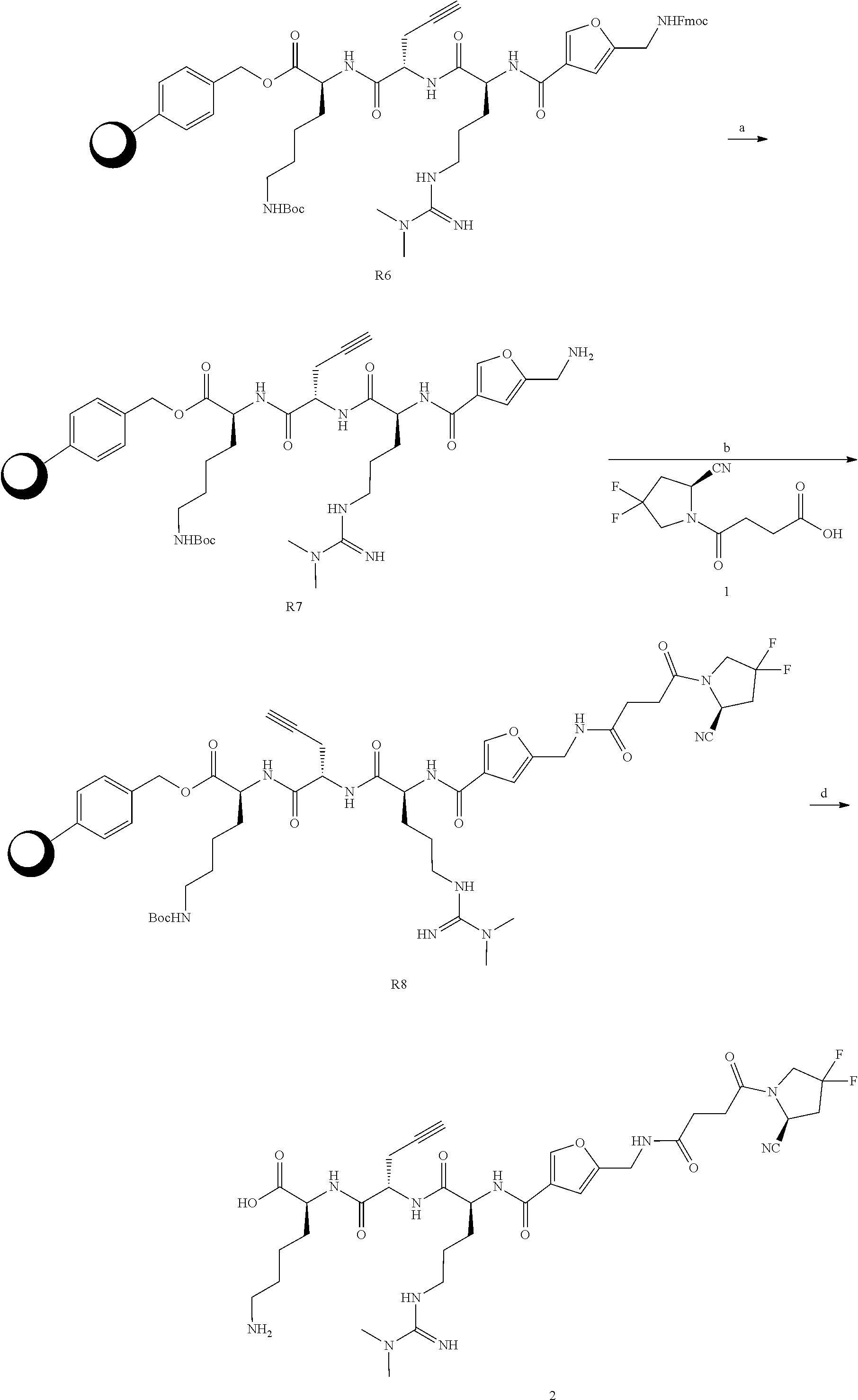

Scheme 1: Reaction scheme for the synthesis of compound 2. Conditions: a) piperidine 205T in N,N-dimethylforwamide, 30′ at r.t.; b) 4 eq. of R—CO₂H, 4 eq. of HATU, 4 eq. of DIPEA in N,N-dimethylforinamide, 1 h at r.t.; c) 4 eq. of R—CO₂H, 4 eq. of EDC, 4 eq. of DIPEA in N,N-dimethylforiaimide, 12 h atr.t.; d) TFA 97.5%, TIPS 2.5%, 2 h atr.t.

Synthesis of compound 2. Commercially available Fmoc-L-Lys(Boc)Wang resin (50 mg, 30 gmol) was deprotected and coupled with Fmoc-L-Propargylglycine (CAS: 198561-07-8, 0.12 mmol, 40 mg) as reported in the General Procedures (1.2.1 and 1.2.2) obtaining the resin R2. After Fmoc-deprotection (1.2.1), Fmoc-Arg(Me)₂-OH (CAS: 268564-10-9, 0.12 mmol, 51 mg) was coupled using the general procedures 1.2.3 to obtain R4. Then, R4 was Fmoc-deprotected (1.2.1) and coupled (1.2.2) with 5-[({[(9H-fluoren-9-yl)methoxy]carbonyl}aimino)methyl]furan-3-carboxylic acid (CAS: 1936714-49-6, 0.12 mmol, 47 mg) to obtain R6. After Fmoc-deprotection (1.2.1), compound 1 (0.12 mmol, 28 mg) was coupled (1.2.2) to obtain R8. The product was cleaved from the resin and then purified using the procedure reported in 1.2.5. The final product (2) was afforded with a yield <500

### 1.3.2 Preparation of Compound 3

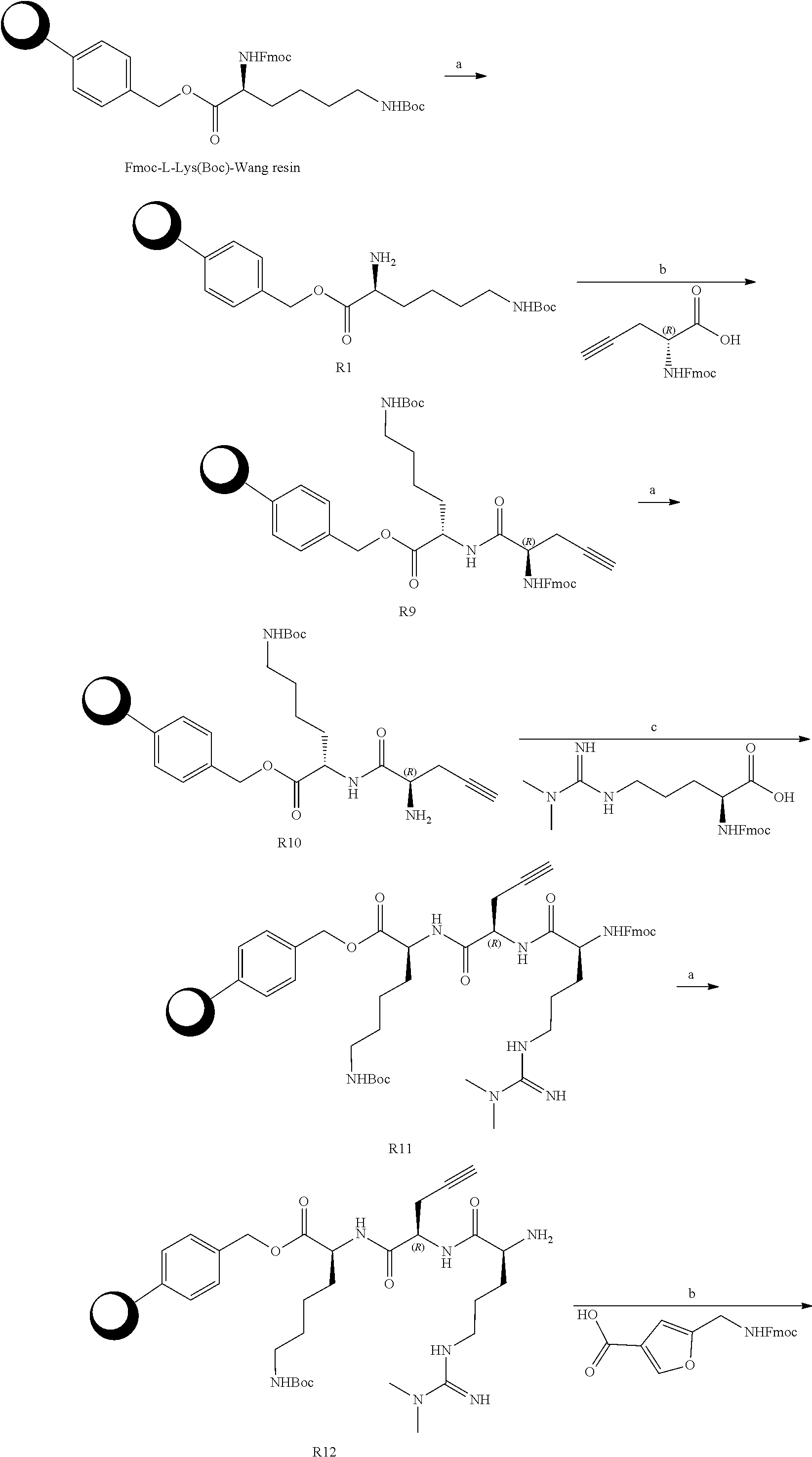

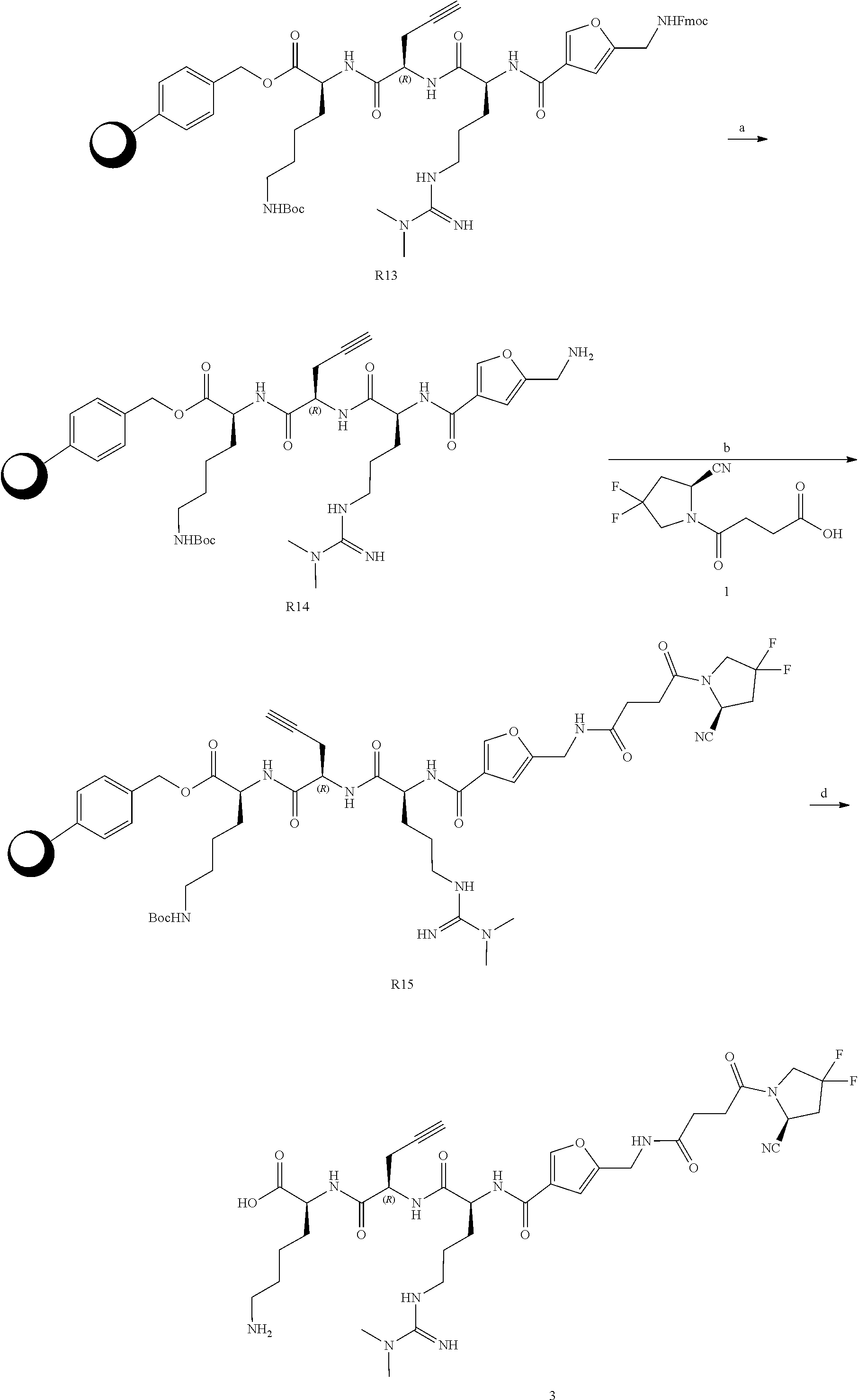

Scheme 2: Reaction scheme for the synthesis of compound 3. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 3. Commercially available Fmoc-L-Lys(Boc)Wang resin (50 mg, 30 gmol) was deprotected and coupled with Fmoc-D-Propargylglycine (CAS: 220497-98-3, 0.12 mmol, 40 mg) as reported in the General Procedures (1.2.1 and 1.2.2) obtaining the resin R9. After Fmoc-deprotection (2.2.1), Fmoc-Arg(Me)₂-OH (CAS: 268564-10-9, 0.12 mmol, 51 mg) was coupled using the general procedures 1.2.3 to obtain R11. Then, R11 was Fmoc-deprotected (1.2.1) and coupled (1.2.2) with 5-[({[(9H-fluoren-9-yl)methoxy]carbonyl}amino)methyl]furan-3-carboxylic acid (CAS: 1936714-49-6, 0.12 mmol, 47 mg) to obtain R13. After Fmoc-deprotection (1.2.1), compound 1 (0.12 mmol, 28 mg) was coupled (1.2.2) to obtain R15. The product was cleaved from the resin and then purified using the procedure reported in 1.2.5. The final product (3) was afforded with a yield <5%.

### 1.3.3 Preparation of Compound 4

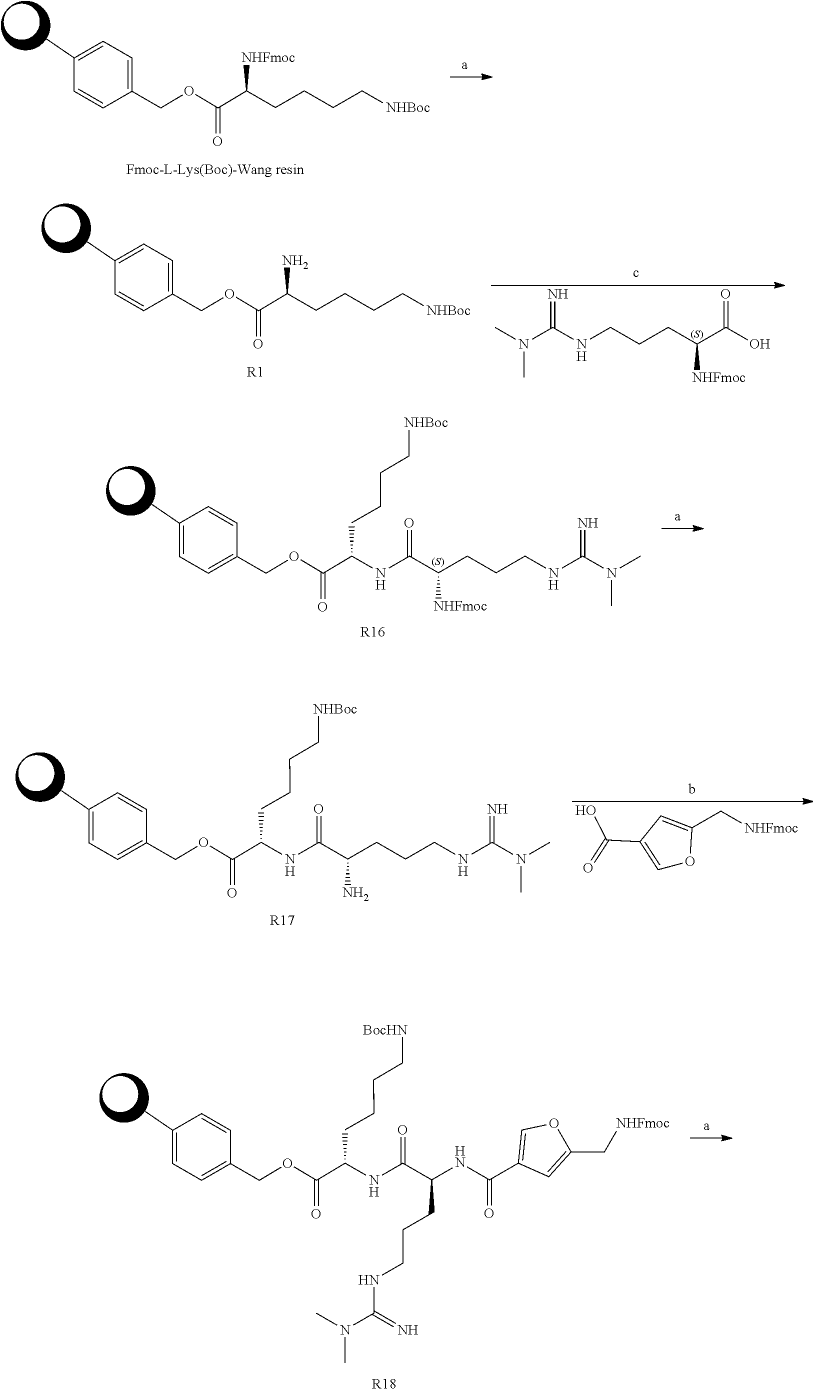

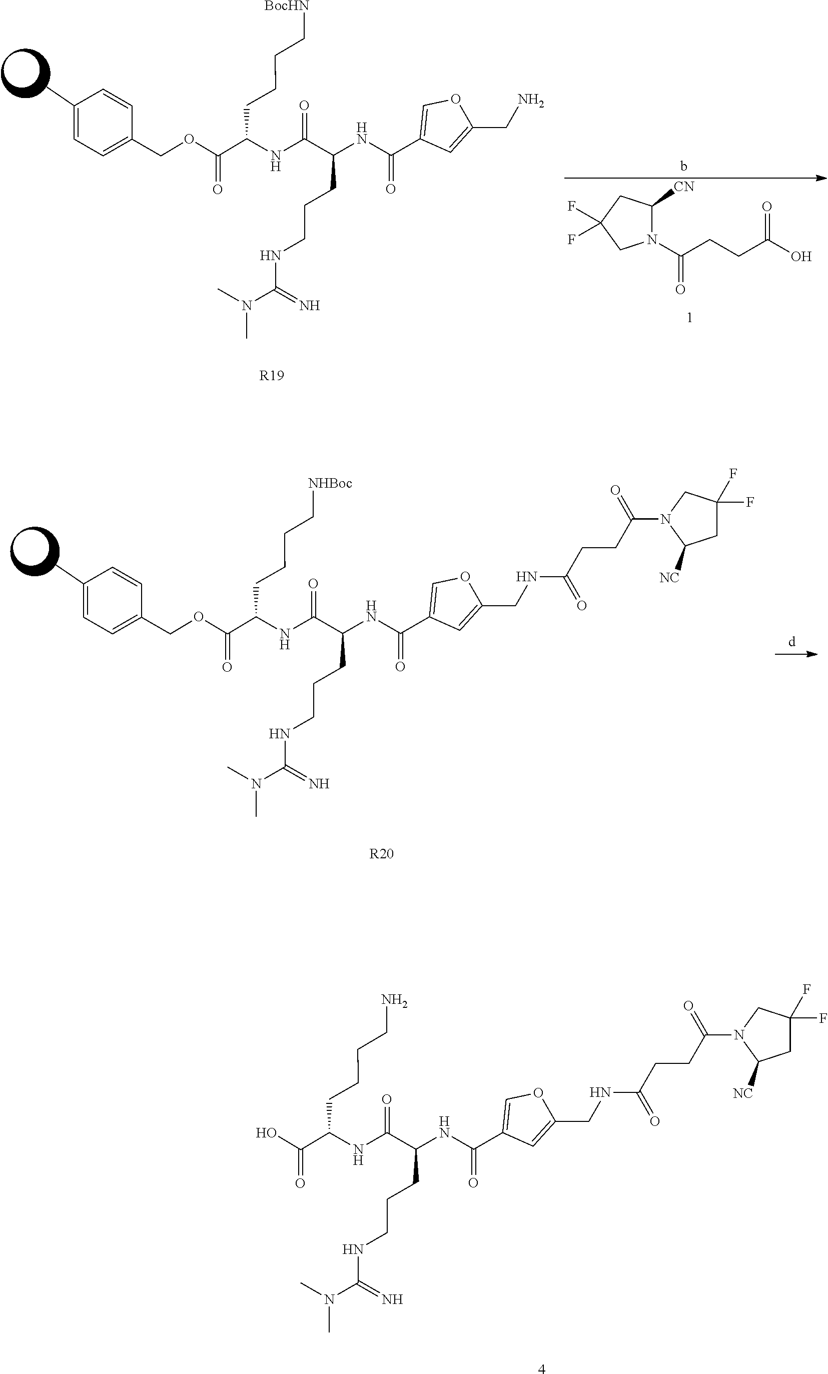

Scheme 3: Reaction scheme for the synthesis of compound 4. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 4. Commercially available Fmoc-L-Lys(Boc)Wang resin (50 mg, 30 gmol) was deprotected and coupled with Fmoc-Arg(Me)₂-OH (CAS: 268564-10-9, 0.12 mmol, 51 mg) using the general procedures (1.2.1 and 1.2.3) to obtain R16. Then, R16 was Fmoc-deprotected (1.2.1) and coupled (1.2.2) with 5-[({[(9H-fluoren-9-yl)methoxy]carbonyl}amino)methyl]furan-3-carboxylic acid (CAS: 1936714-49-6, 0.12 mmol, 47 mg) to obtain R18. After Fmoc-deprotection (1.2.1), compound 1 (0.12 mmol, 28 mg) was coupled (1.2.2) to obtain R20. The product was cleaved from the resin and then purified using the procedure reported in 1.2.5. The final product (4) was afforded with a yield <5%.

### 1.3.4 Preparation of Compound 5

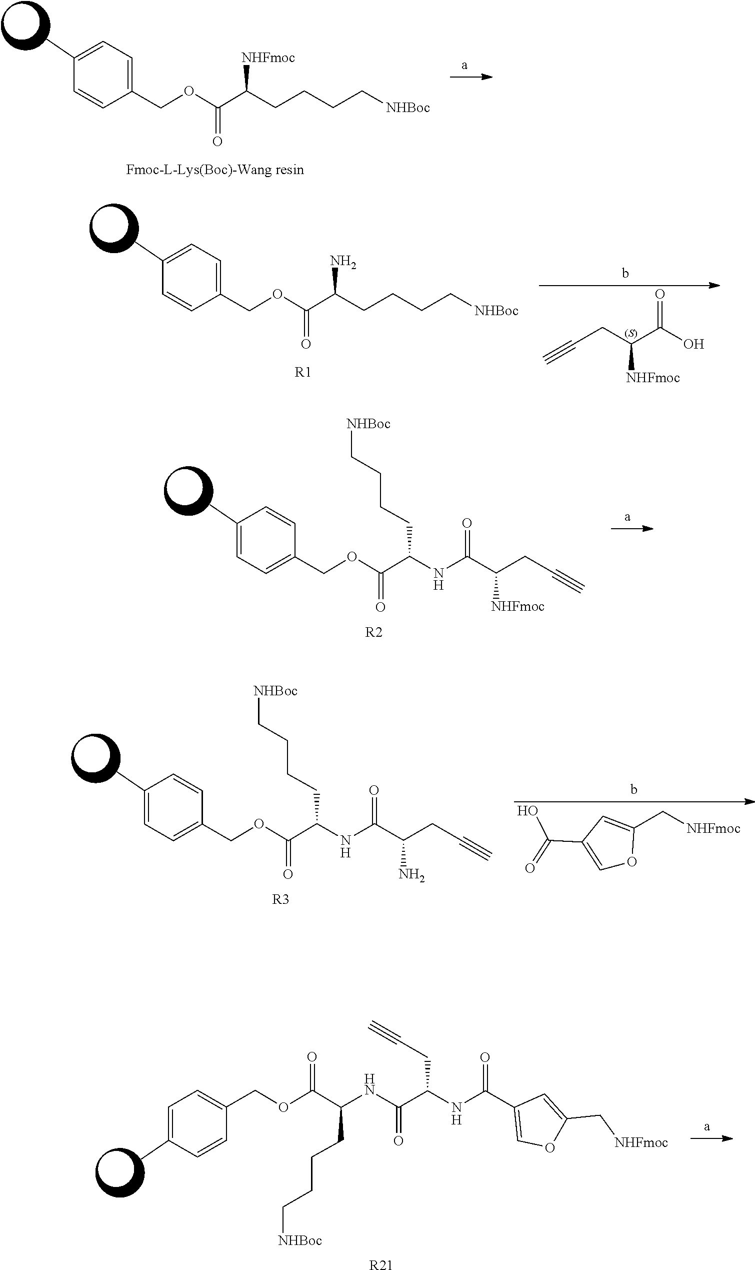

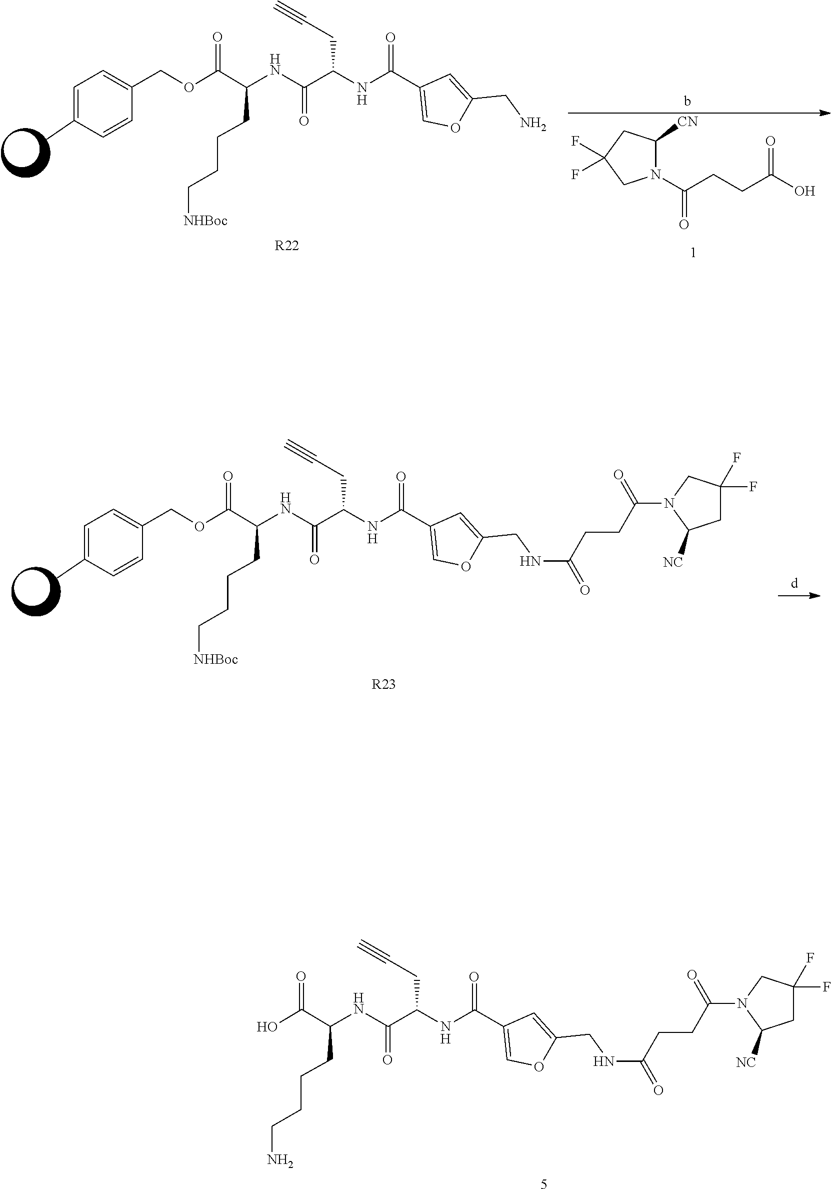

Scheme 4: Reaction scheme for the synthesis of compound 5. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; d) TFA 97.5%, TIPS 2.5%, 2 h at r.t.

Synthesis of compound 5. Commercially available Fmoc-L-Lys(Boc)Wang resin (50 mg, 30 gmol) was deprotected and coupled with Fmoc-L-Propargylglycine (CAS: 198561-07-8, 0.12 mmol, 40 mg) as reported in the General Procedures (1.2.1 and 1.2.2) obtaining the resin R2. After Fmoc-deprotection (1.2.1), 5-[({[(9H-fluoren-9-yl)methoxy]carbonyl}amino)methyl]furan-3-carboxylic acid (CAS: 1936714-49-6, 0.12 mmol, 47 mg) was coupled as described in 1.2.2 to obtain R21. After Fmoc-deprotection (1.2.1), compound 1 (0.12 mmol, 28 mg) was coupled (1.2.2) to obtain R23. The product was cleaved from the resin and then purified using the procedure reported in 1.2.5. The final product (5) was afforded with a yield <5%.

### 1.3.5 Preparation of Compound 6

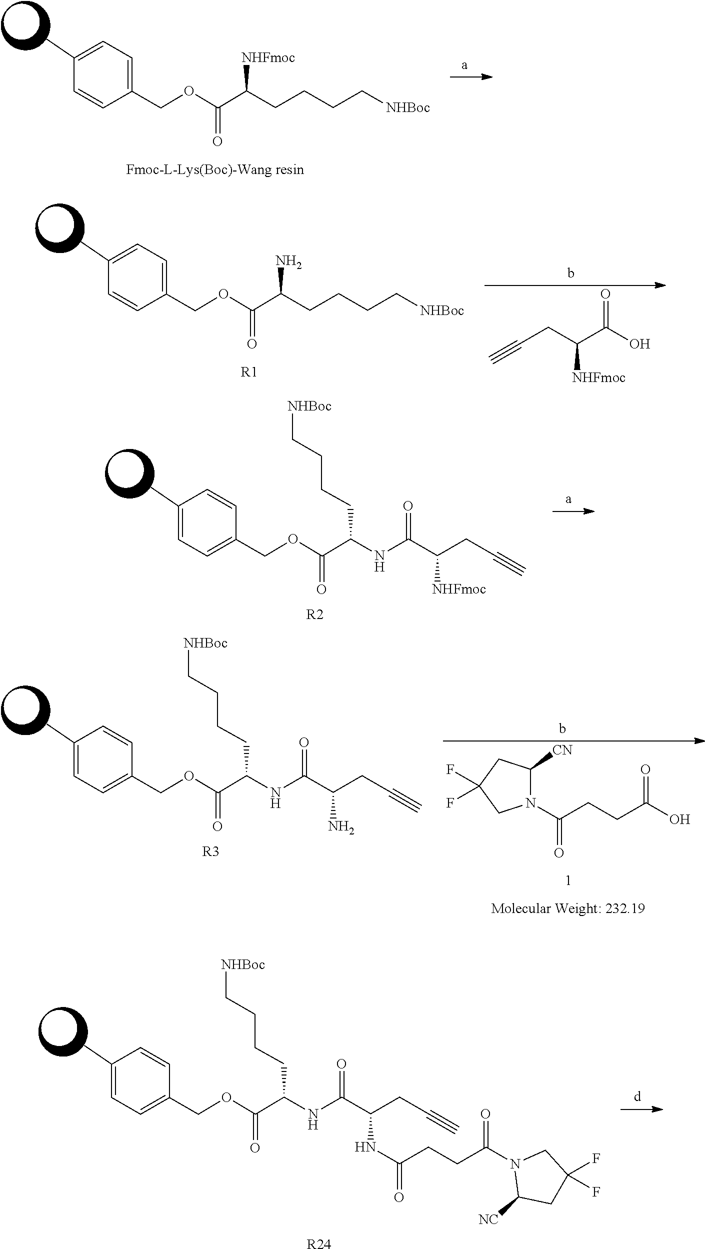

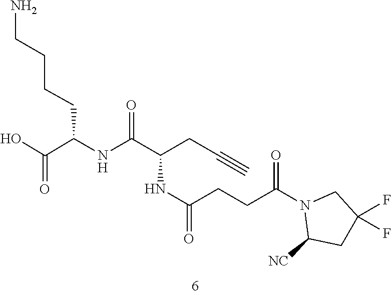 [SMILES: C#CC[C@H](NC(=O)CCC(=O)N1CC(F)(F)C[C@H]1C#N)C(=O)N[C@@H](CCCCN)C(=O)O ; "6"]

Scheme 5: Reaction scheme for the synthesis of compound 6. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; d) TFA 97.5%, TIPS 2.5%, 2 h at r.t.

Synthesis of compound 6. Commercially available Fmoc-L-Lys(Boc)Wang resin (50 mg, 30 gmol) was deprotected and coupled with Fmoc-L-Propargylglycine (CAS: 198561-07-8, 0.12 mmol, 40 mg) as reported in the General Procedures (1.2.1 and 1.2.2) obtaining the resin R2. After Fmoc-deprotection (1.2.1), compound 1 (0.12 mmol, 28 mg) was coupled (1.2.2) to obtain R24. The product was cleaved from the resin and then purified using the procedure reported in 1.2.5. The final product (6) was afforded with a yield <5%.

### 1.3.6 Preparation of Compound 9

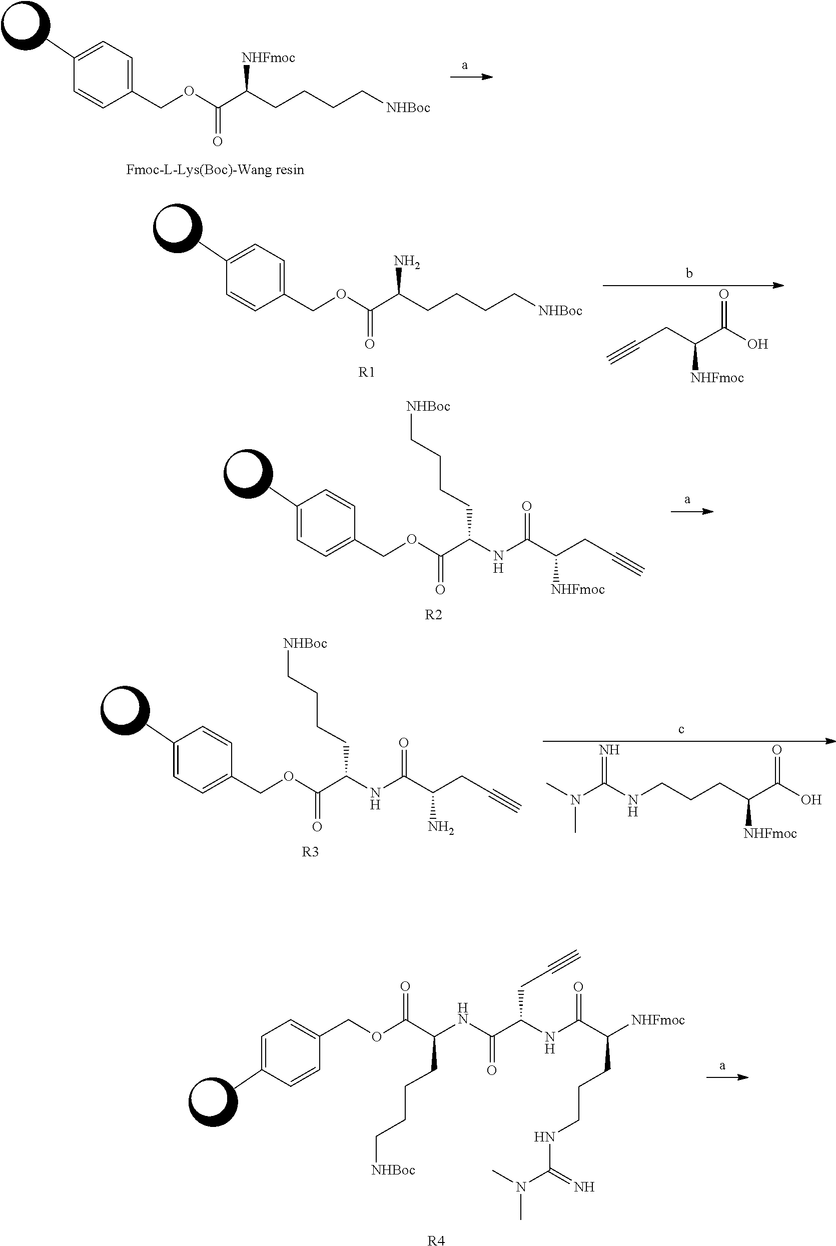

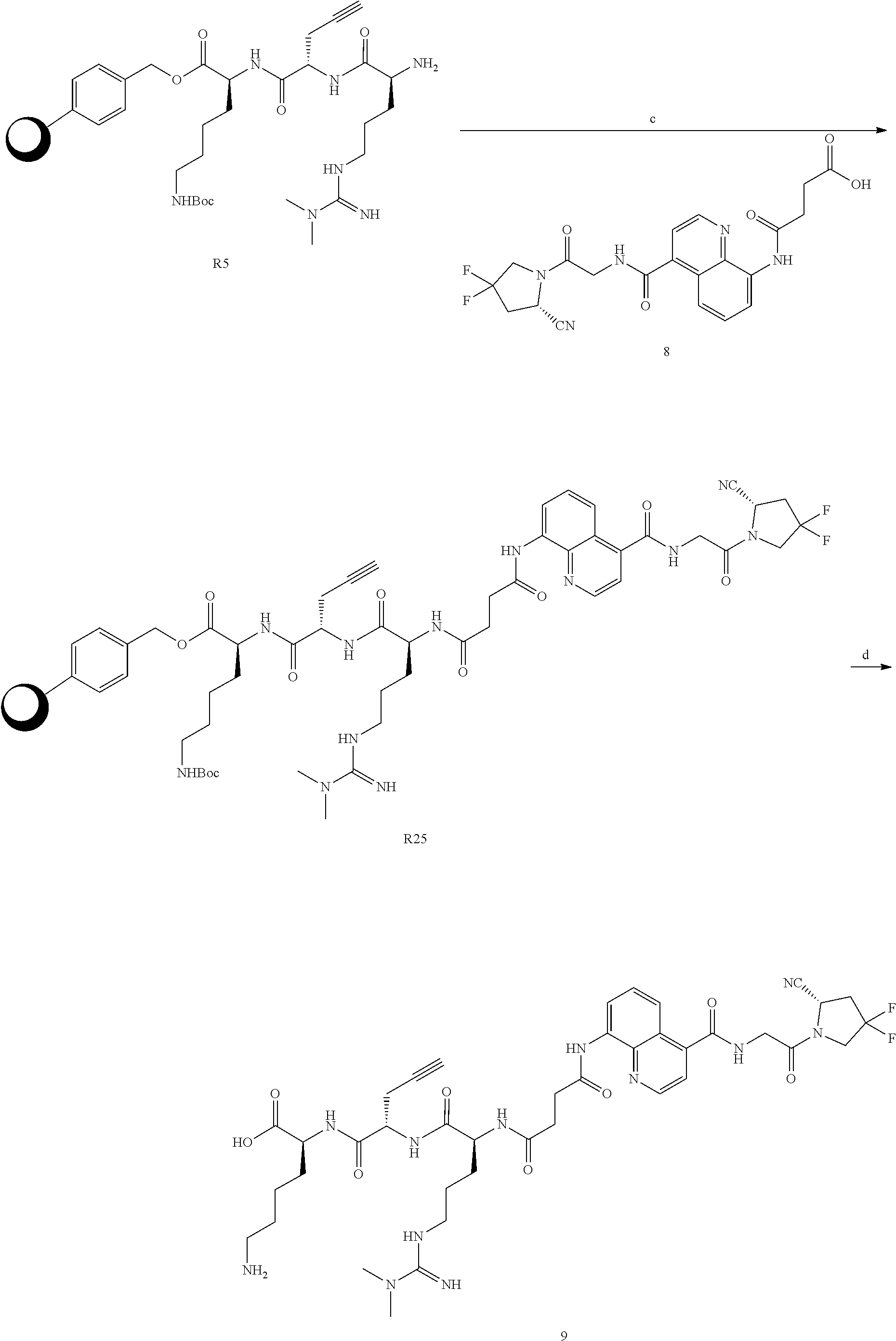

Scheme 6: Reaction scheme for the synthesis of compound 9. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 9. Commercially available Fmoc-L-Lys(Boc)Wang resin (50 mg, 30 gmol) was deprotected and coupled with Fmoc-L-Propargylglycine (CAS: 198561-07-8, 0.12 mmol, 40 mg) as reported in the General Procedures (1.2.1 and 1.2.2) obtaining the resin R2. After Fmoc-deprotection (1.2.1), Fmoc-Arg(Me)₂-OH (CAS: 268564-10-9, 0.12 mmol, 51 mg) was coupled using the general procedures 1.2.3 to obtain R4. Then, R4 was Fmoc-deprotected (1.2.1) and coupled (1.2.3) with compound 8 (0.12 mmol, 55 mg) to obtain R25. The product was cleaved from the resin and then purified using the procedure reported in 1.2.5. The final product (9) was afforded with a yield <5%.

### 1.3.7 Preparation of Compound 10

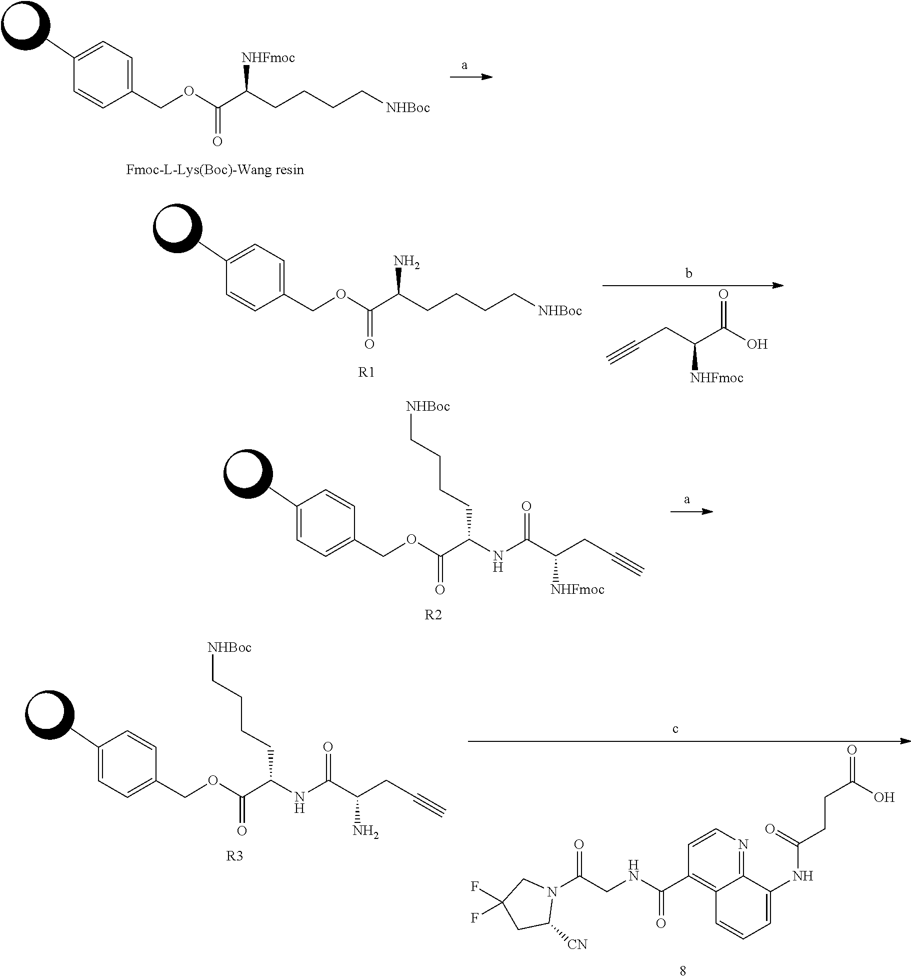

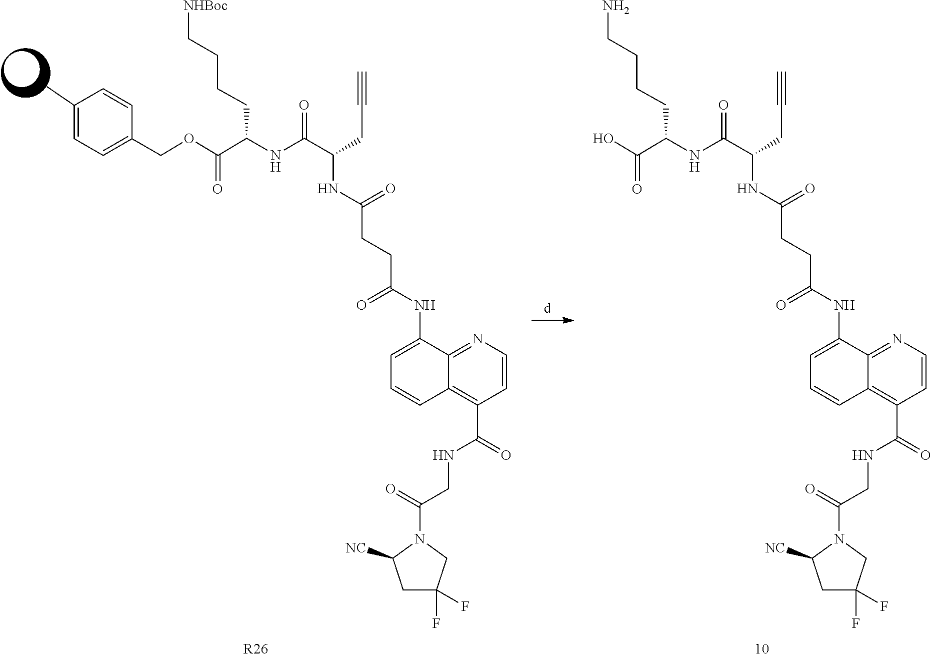

Scheme 7: Reaction scheme for the synthesis of compound 10. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 10. Commercially available Fmoc-L-Lys(Boc)Wang resin (50 mg, 30 gmol) was deprotected and coupled with Fmoc-L-Propargylglycine (CAS: 198561-07-8, 0.12 mmol, 40 mg) as reported in the General Procedures (1.2.1 and 1.2.2) obtaining the resin R2. After Fmoc-deprotection (1.2.1), compound 8 (0.12 mmol, 55 mg) was coupled (1.2.3) to obtain R26. The product was cleaved from the resin and then purified using the procedure reported in 1.2.5. The final product (10) was afforded with a yield <5%.

### 1.3.8 Preparation of Compound 11

### 1.3.8.1 Synthesis of Compound 11

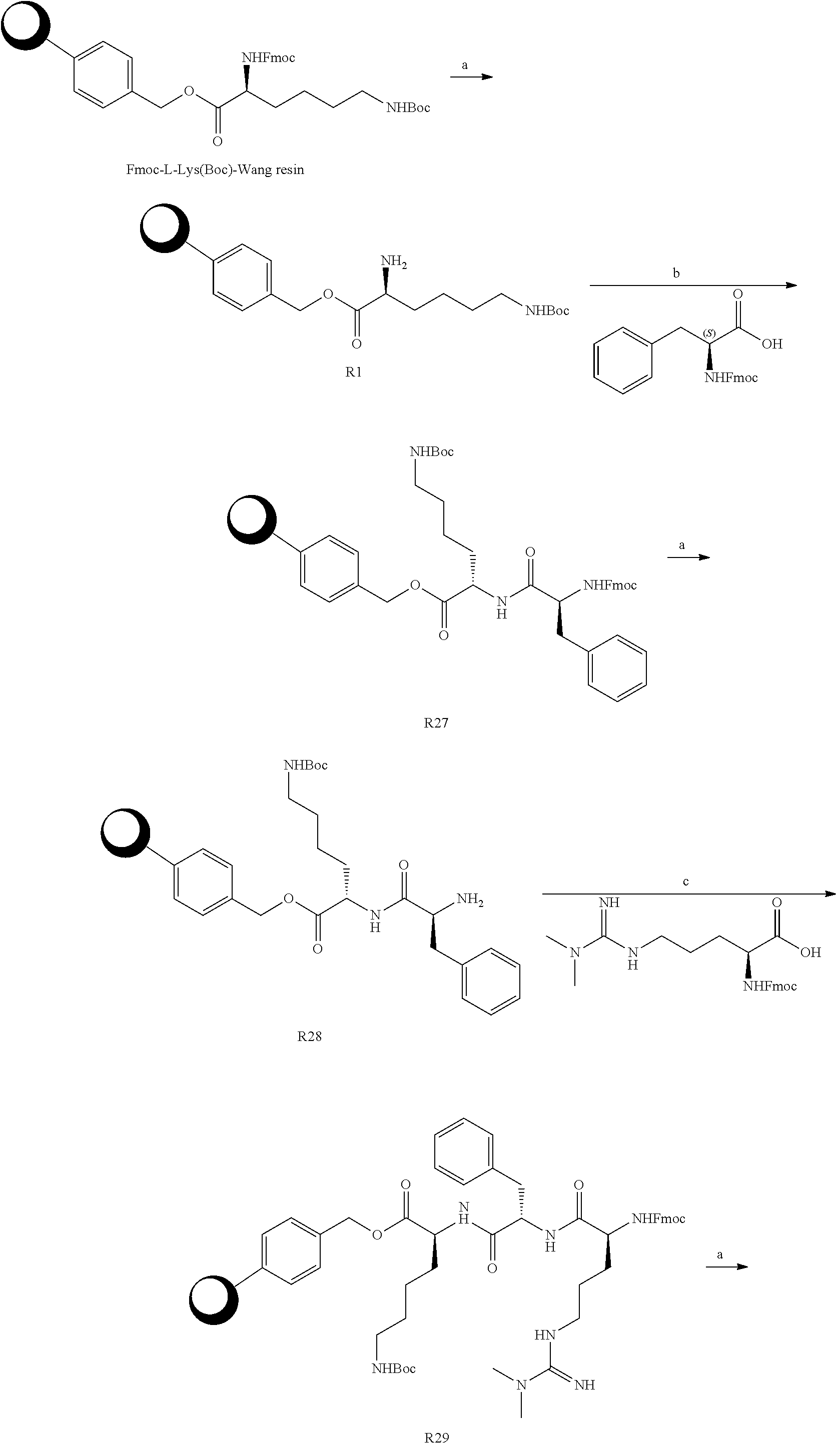

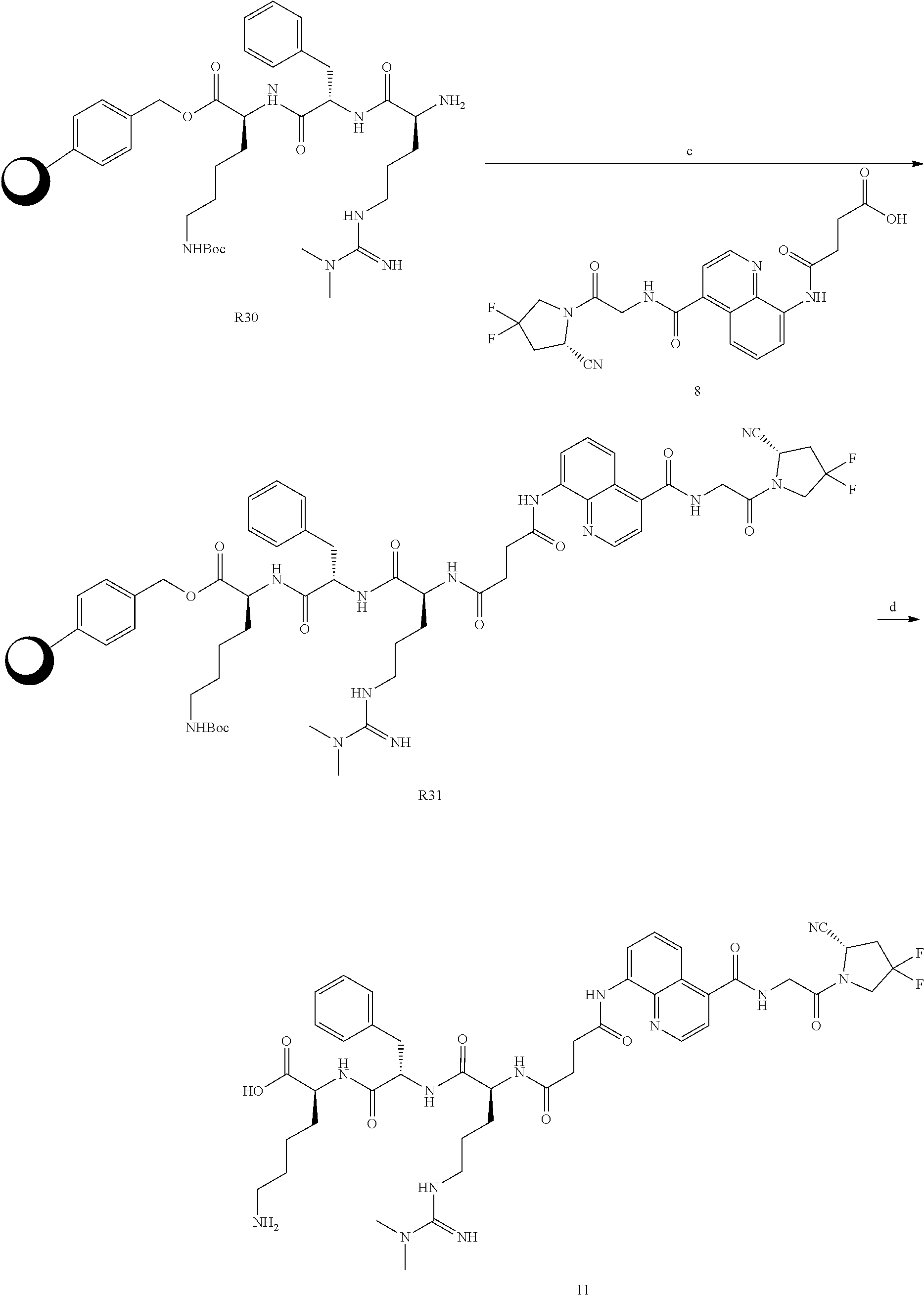

Scheme 8: Reaction scheme for the synthesis of compound 11. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 11. Commercially available Fmoc-L-Lys(Boc)Wang resin (50 mg, 30 gmol) was deprotected and coupled with Fmoc-L-phenylalanine (CAS: 35661-40-6, 0.12 mmol, 46 mg) as reported in the General Procedures (1.2.1 and 1.2.2) obtaining the resin R27. After Fmoc-deprotection (1.2.1), Fmoc-Arg(Me)₂-OH (CAS: 268564-10-9, 0.12 mmol, 51 mg) was coupled using the general procedures 1.2.3 to obtain R29. Then, R29 was Fmoc-deprotected (1.2.1) and coupled (1.2.3) with compound 8 (0.12 mmol, 55 mg) to obtain R31. The product was cleaved from the resin and then purified using the procedure reported in 1.2.5. The final product (11) was afforded with a yield <5%.

### 1.3.8.2 Alternative Synthesis of Compound 11

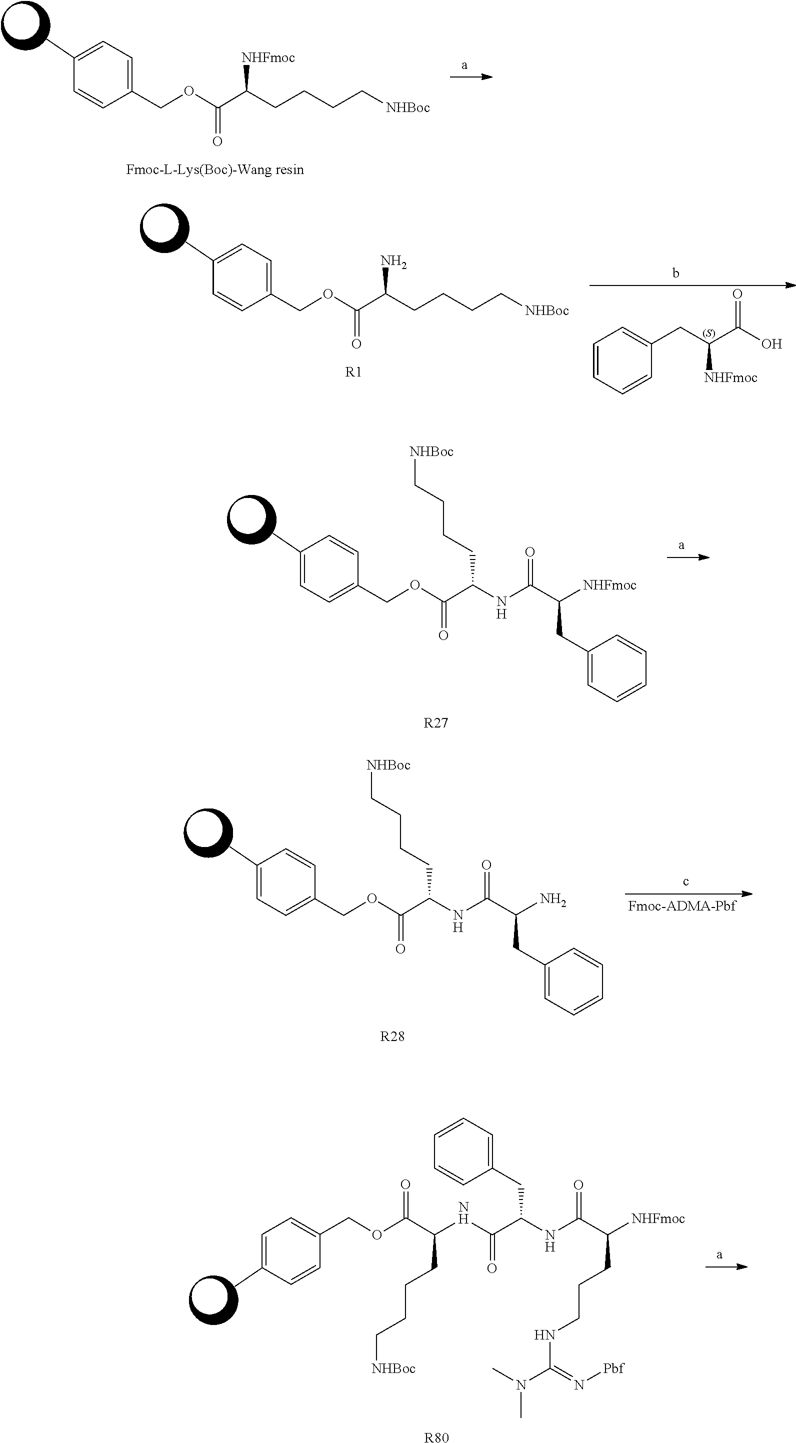

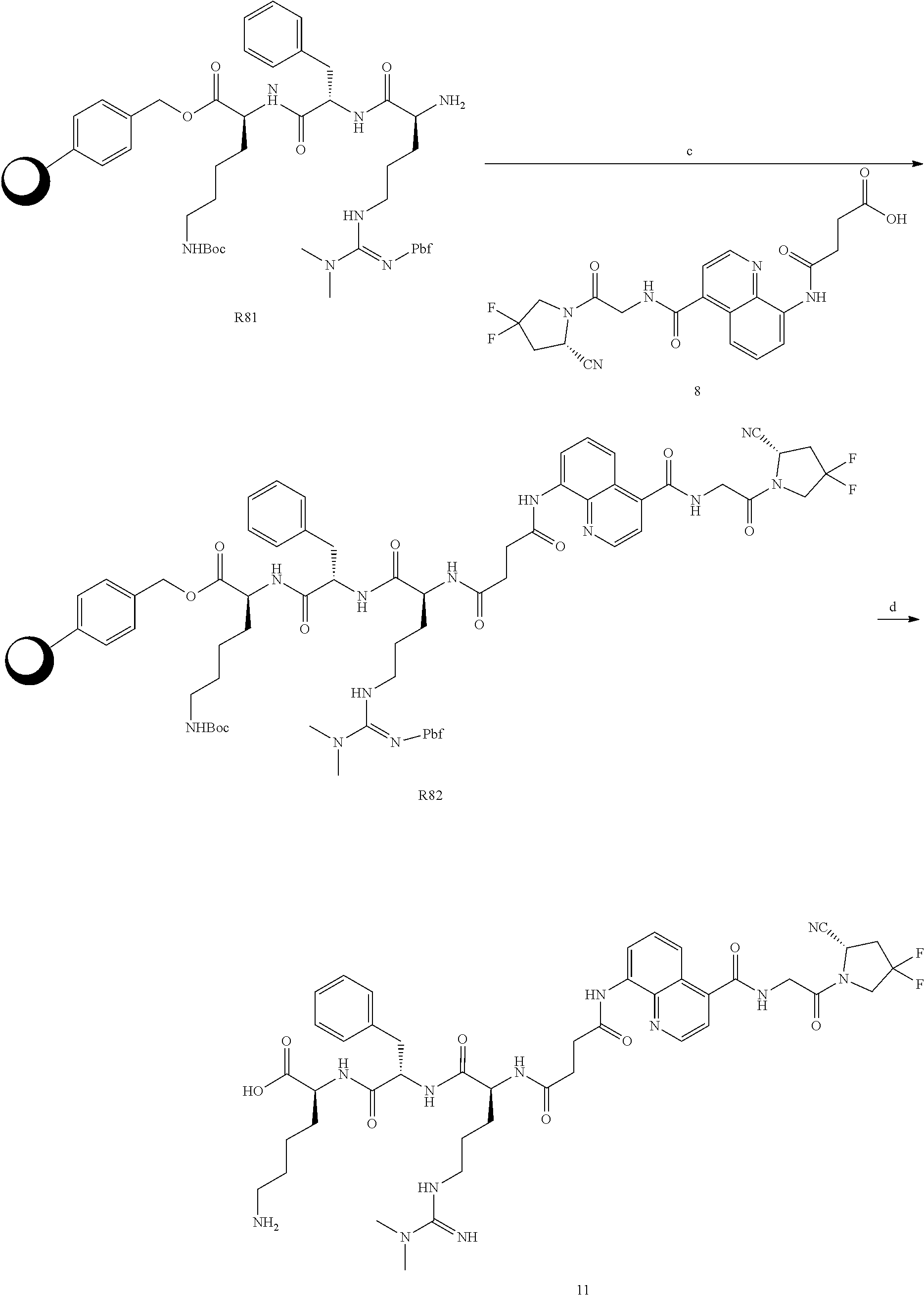

Scheme 9: Reaction scheme for the synthesis of compound 11. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; d) TFA 97.5%, TIPS 2.5%, 2 h at r.t.

Alternative synthesis of compound 11. Commercially available Fmoc-L-Lys(Boc)Wang resin (50 mg, 30 gmol) was deprotected and coupled with Fmoc-L-phenylalanine (CAS: 35661-40-6, 0.12 mmol, 46 mg) as reported in the General Procedures (1.2.1 and 1.2.2) obtaining the resin R27. After Fmoc-deprotection (1.2.1), Fmoc-ADMA(Pbf)-OH (0.12 mmol, 81 mg) was coupled using the general procedures 1.2.2 to obtain R80. Then, R80 was Fmoc-deprotected (1.2.1) and coupled (1.2.2) with compound 8 (0.12 mmol, 55 mg) to obtain R82. The product was cleaved from the resin and then purified using the procedure reported in 1.2.5. The final product (11) was afforded with a yield <5%.

The second route with the Pbf protecting group on the dimethyl arginine moiety helps the synthesis in solid phase. It prevents the arginine to rearrange as a lactame and increases the yield of the overall reaction avoiding the formation of undesired side products and letting the arginine derivative to be more reactive. This synthetic route can be applied particularly advantageously, e.g., for the synthesis compounds 2, 3, 4, 9, 11, 34 and 37 (last two have already the alternative route in the synthetic scheme). Moreover, all the rest with the natural arginine (compounds 12, 13, 14, 15, 16, 17, 18, 19) can benefit of the same procedure with the Pbf-protected natural arginine.

### Preparation of Compound 12

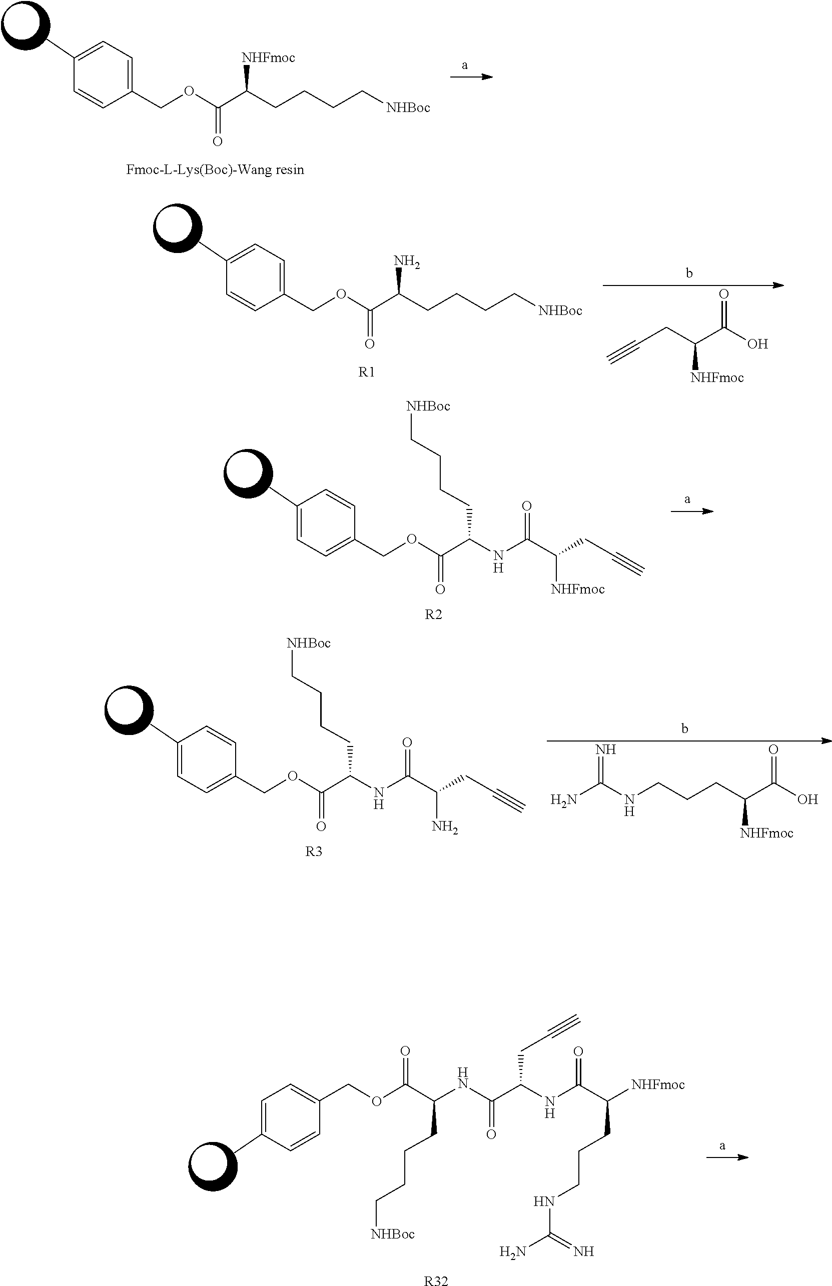

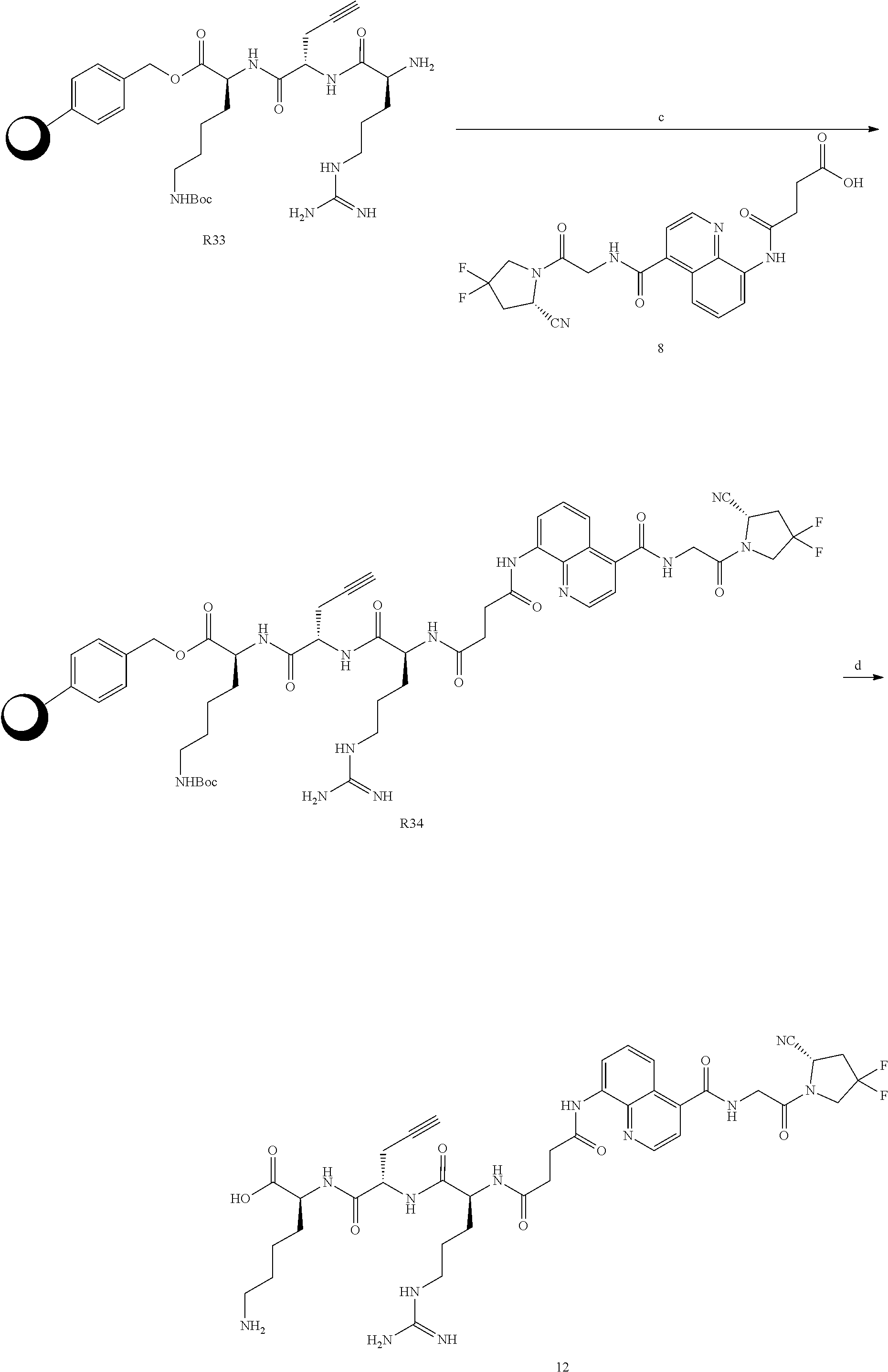

Scheme 10: Reaction scheme for the synthesis of compound 12. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 12. Commercially available Fmoc-L-Lys(Boc)Wang resin (50 mg, 30 gmol) was deprotected and coupled with Fmoc-L-Propargylglycine (CAS: 198561-07-8, 0.12 mmol, 40 mg) as reported in the General Procedures (1.2.1 and 1.2.2) obtaining the resin R2. After Fmoc-deprotection (1.2.1), Fmoc-L-Arginine (CAS: 91000-69-0, 0.12 mmol, 48 mg) was coupled as reported in 1.2.2 to afford the resin R32. R32 was then Fmoc-deprotected (1.2.1) and compound 8 (0.12 mmol, 55 mg) was coupled (1.2.3) to obtain R34. The product was cleaved from the resin and then purified using the procedure reported in 1.2.5. The final product (12) was afforded with a yield <5%.

### 1.3.9 Preparation of Compound 13

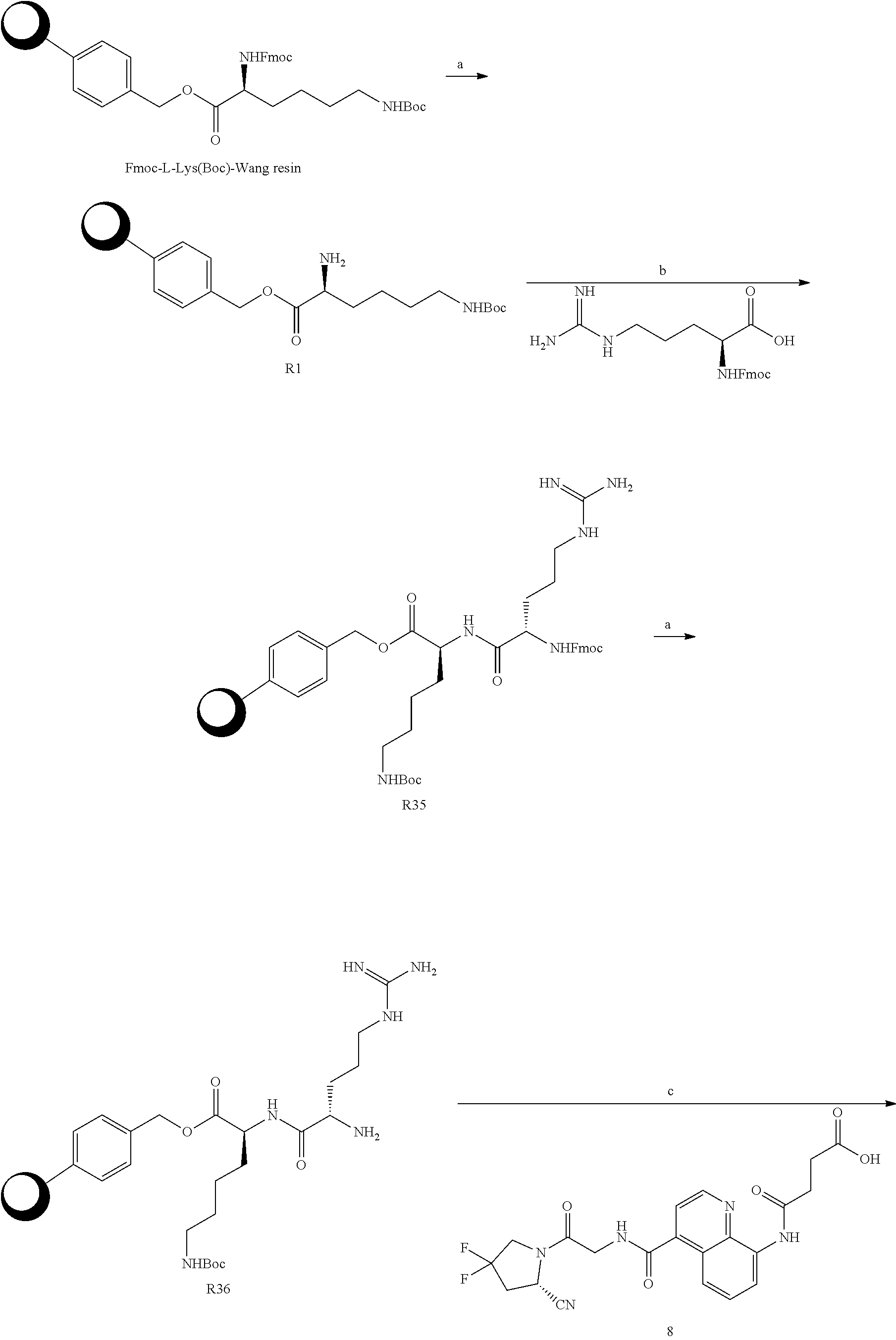

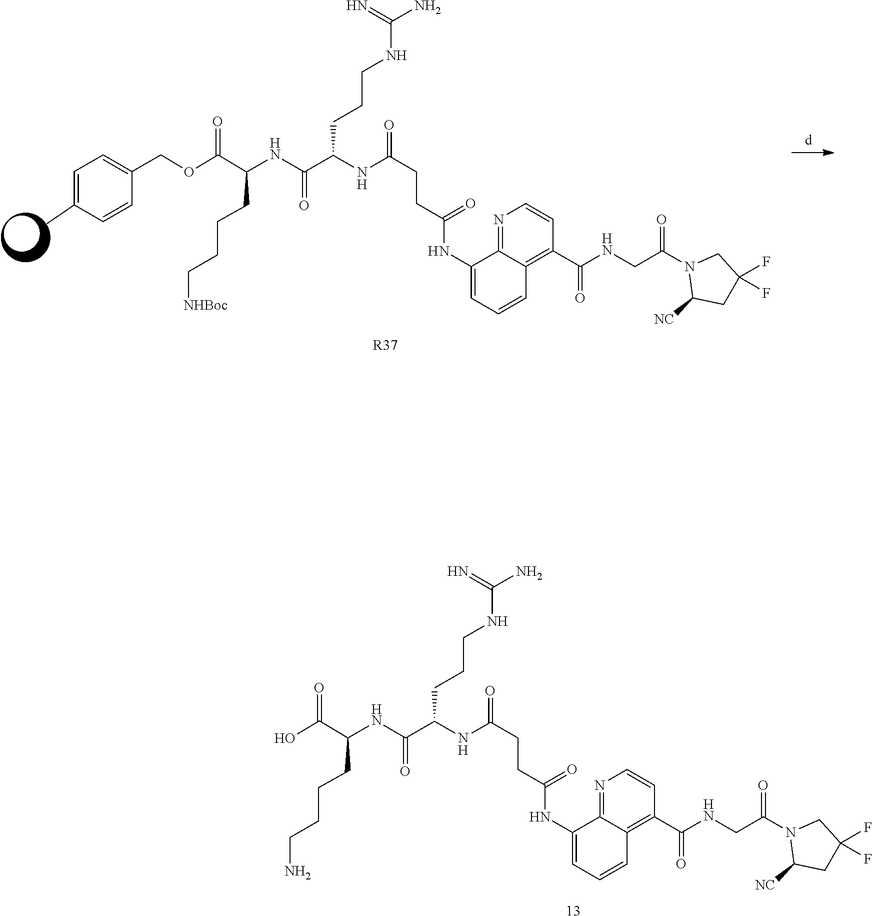

Scheme 11: Reaction scheme for the synthesis of compound 13. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 13. Commercially available Fmoc-L-Lys(Boc)Wang resin (50 mg, 30 gmol) was deprotected and coupled with Fmoc-L-Arginine (CAS: 91000-69-0, 0.12 mmol, 48 mg) as reported in 1.2.2 to obtain the resin R35. R35 was then Fmoc-deprotected and coupled with compound 8 (0.12 mmol, 55 mg) (1.2.3) to obtain R37. The product was cleaved from the resin and then purified using the procedure reported in 1.2.5. The final product (13) was afforded with a yield <5%.

### 1.3.10 Preparation of Compound 14

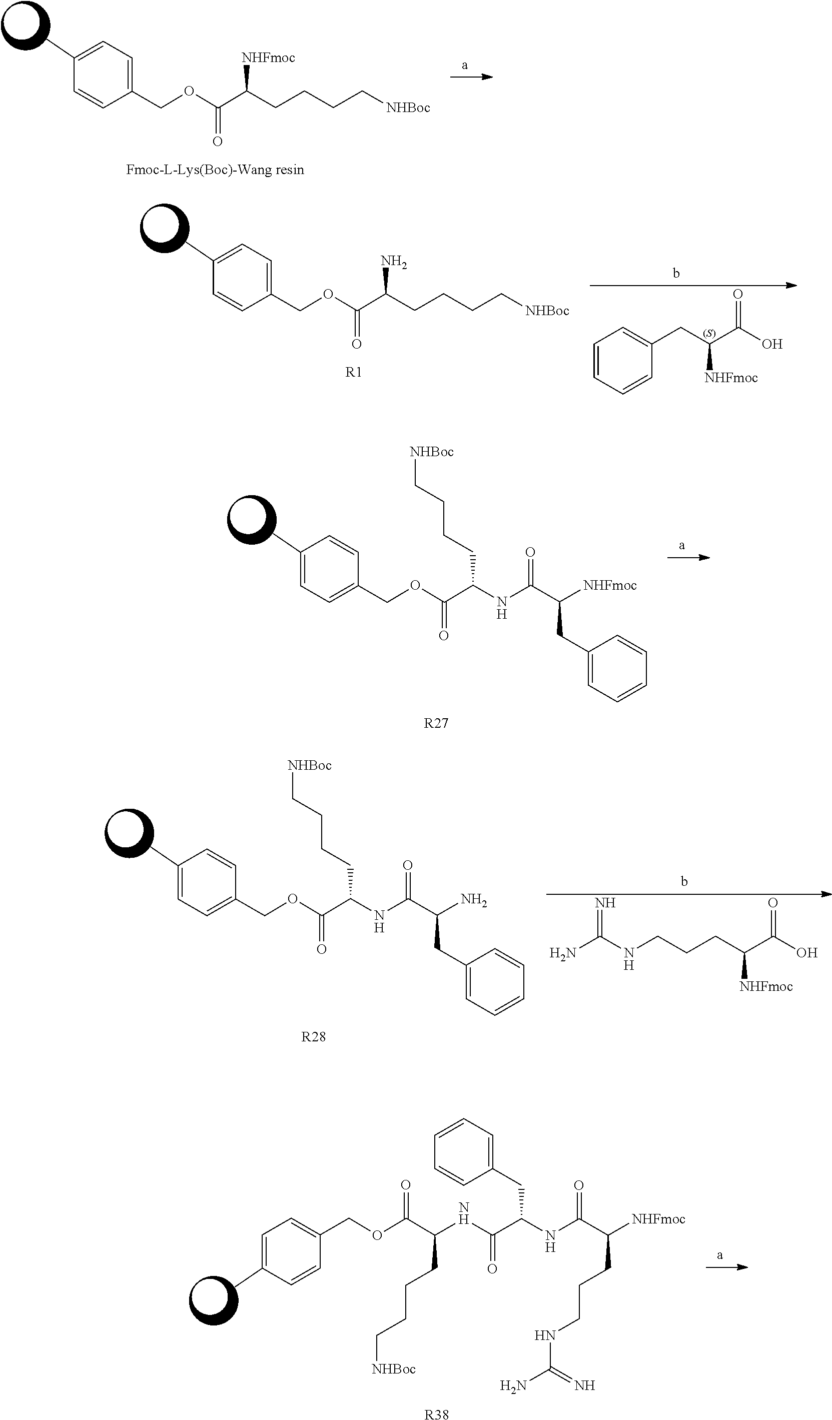

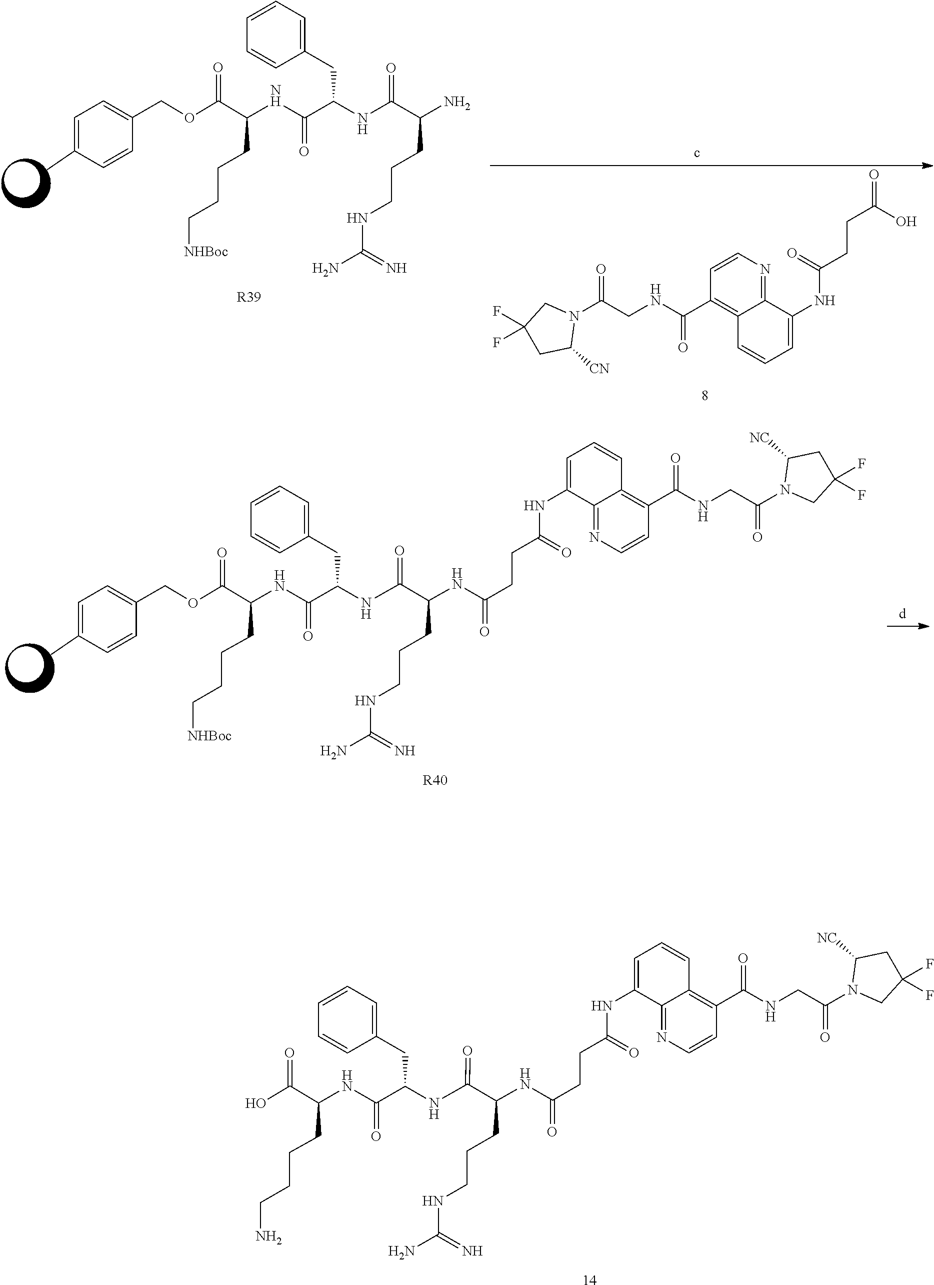

Scheme 12: Reaction scheme for the synthesis of compound 14. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 14. Commercially available Fmoc-L-Lys(Boc)Wang resin (50 mg, 30 gmol) was deprotected and coupled with Fmoc-L-phenylalanine (CAS: 35661-40-6, 0.12 mmol, 46 mg) as reported in the General Procedures (1.2.1 and 1.2.2) obtaining the resin R27. After Fmoc-deprotection (1.2.1), Fmoc-L-Arginine (CAS: 91000-69-0, 0.12 mmol, 48 mg) was coupled using the general procedures 1.2.2 to obtain R38. Then, R38 was Fmoc-deprotected (1.2.1) and coupled (1.2.3) with compound 8 (0.12 mmol, 55 mg) to obtain R40. The product was cleaved from the resin and then purified using the procedure reported in 1.2.5. The final product (14) was afforded with a yield <5%.

### 1.3.11 Preparation of Compound 15

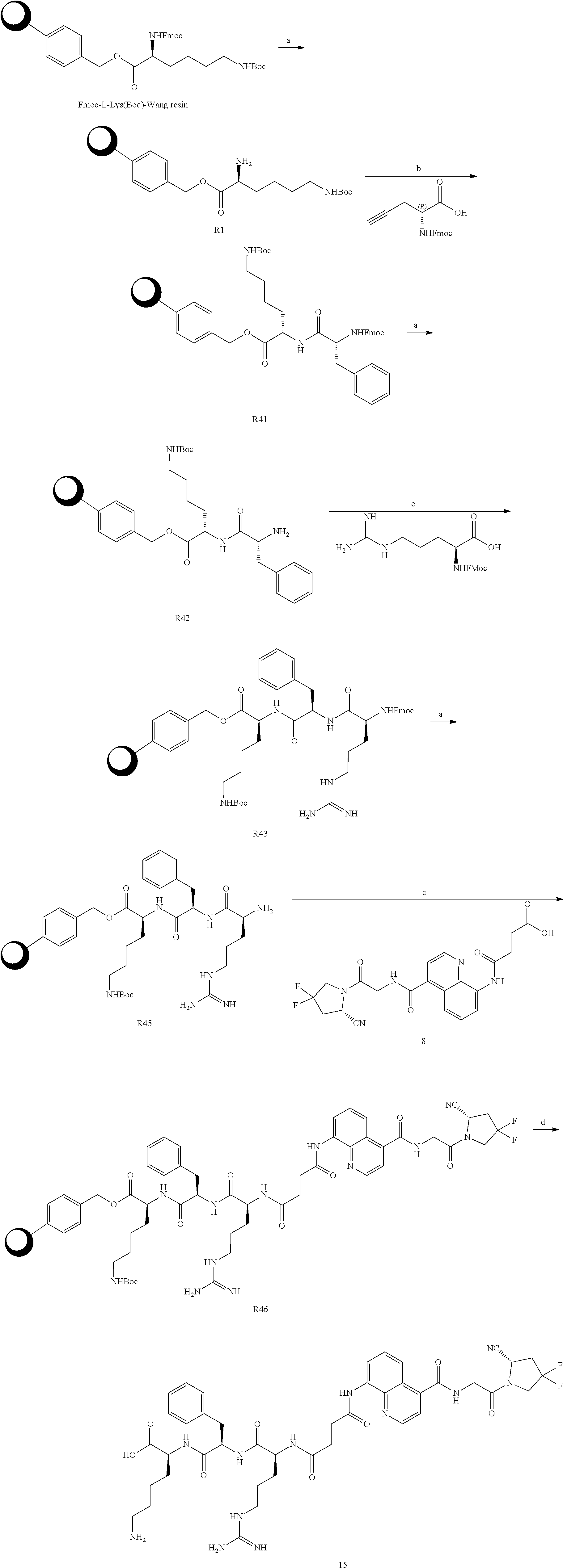

Scheme 13: Reaction scheme for the synthesis of compound 15. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 15. Commercially available Fmoc-L-Lys(Boc)Wang resin (50 mg, 30 gmol) was deprotected and coupled with Fmoc-D-phenylalanine (CAS: 86123-10-6, 0.12 mmol, 46 mg) as reported in the General Procedures (1.2.1 and 1.2.2) obtaining the resin R41. After Fmoc-deprotection (1.2.1), Fmoc-L-Arginine (CAS: 91000-69-0, 0.12 mmol, 48 mg) was coupled using the general procedures 1.2.2 to obtain R43. Then, R43 was Fmoc-deprotected (1.2.1) and coupled (1.2.3) with compound 8 (0.12 mmol, 55 mg) to obtain R46. The product was cleaved from the resin and then purified using the procedure reported in 1.2.5. The final product (15) was afforded with a yield <5%.

### 1.3.12 Preparation of Compound 16

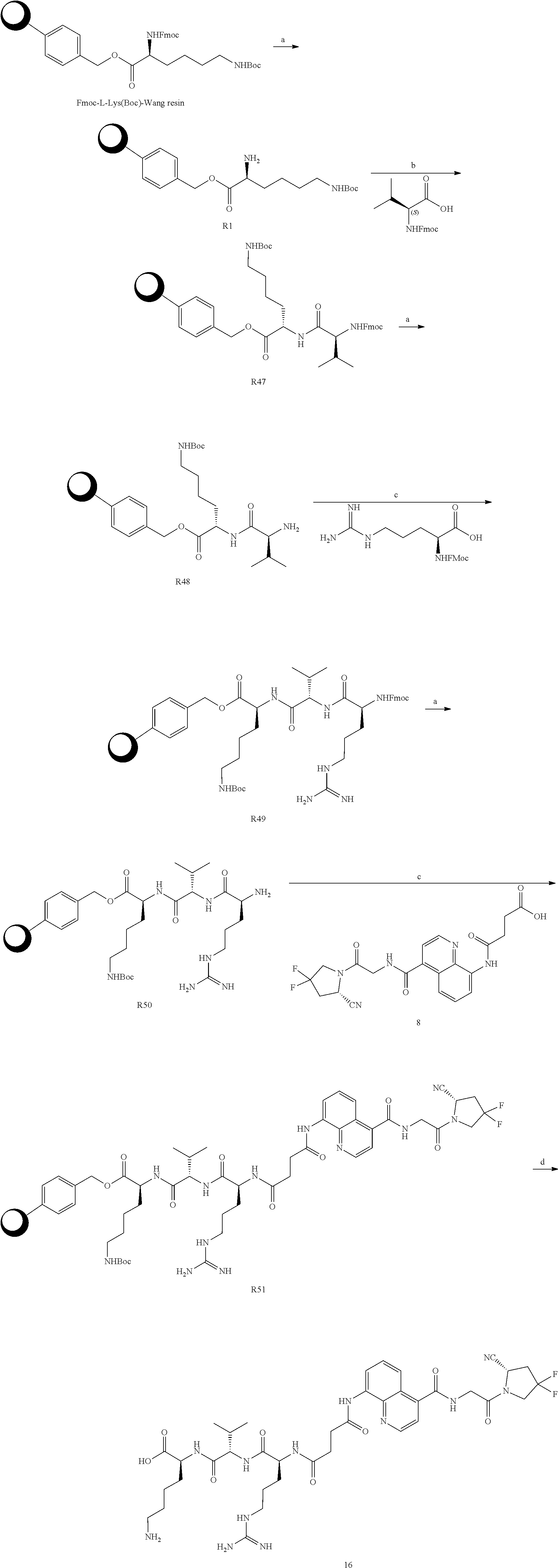

Scheme 14: Reaction scheme for the synthesis of compound 16. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 16. Commercially available Fmoc-L-Lys(Boc)Wang resin (50 mg, 30 gmol) was deprotected and coupled with Fmoc-L-valine (CAS: 68858-20-8, 0.12 mmol, 41 mg) as reported in the General Procedures (1.2.1 and 1.2.2) obtaining the resin R47. After Fmoc-deprotection (1.2.1), Fmoc-L-Arginine (CAS: 91000-69-0, 0.12 mmol, 48 mg) was coupled using the general procedures 1.2.2 to obtain R49. Then, R49 was Fmoc-deprotected (1.2.1) and coupled (1.2.3) with compound 8 (0.12 mmol, 55 mg) to obtain R51. The product was cleaved from the resin and then purified using the procedure reported in 1.2.5. The final product (16) was afforded with a yield <5%.

### 1.3.13 Preparation of Compound 17

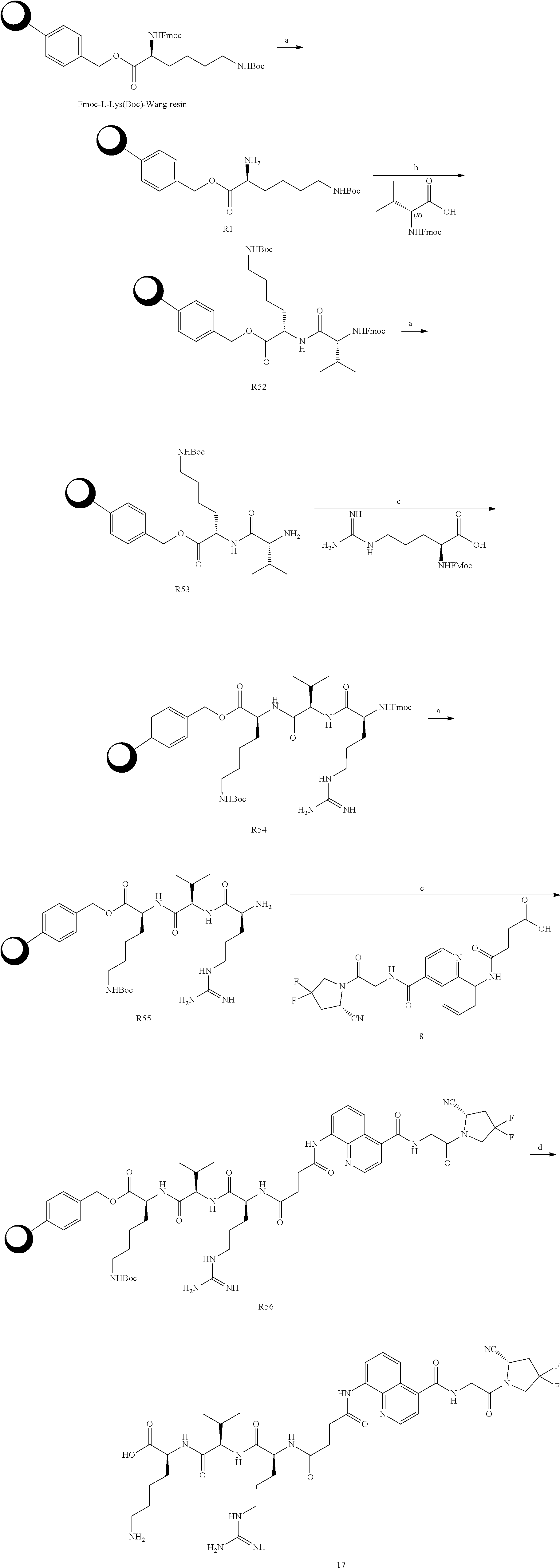

Scheme 15: Reaction scheme for the synthesis of compound 17. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h at r.t.; d) TFA 97.5%, TIPS 2.5%, 2 h at r.t.

Synthesis of compound 17. Commercially available Fmoc-L-Lys(Boc)Wang resin (50 mg, 30 μmol) was deprotected and coupled with Fmoc-D-valine (CAS: 84624-17-9, 0.12 mmol, 41 mg) as reported in the General Procedures (1.2.1 and 1.2.2) obtaining the resin R52. After Fmoc-deprotection (1.2.1), Fmoc-L-Arginine (CAS: 91000-69-0, 0.12 mmol, 48 mg) was coupled using the general procedures 1.2.2 to obtain R54. Then, R54 was Fmoc-deprotected (1.2.1) and coupled (1.2.3) with compound 8 (0.12 mmol, 55 mg) to obtain R56. The product was cleaved from the resin and then purified using the procedure reported in 1.2.5. The final product (17) was afforded with a yield <5%.

### 1.3.14 Preparation of Compound 18

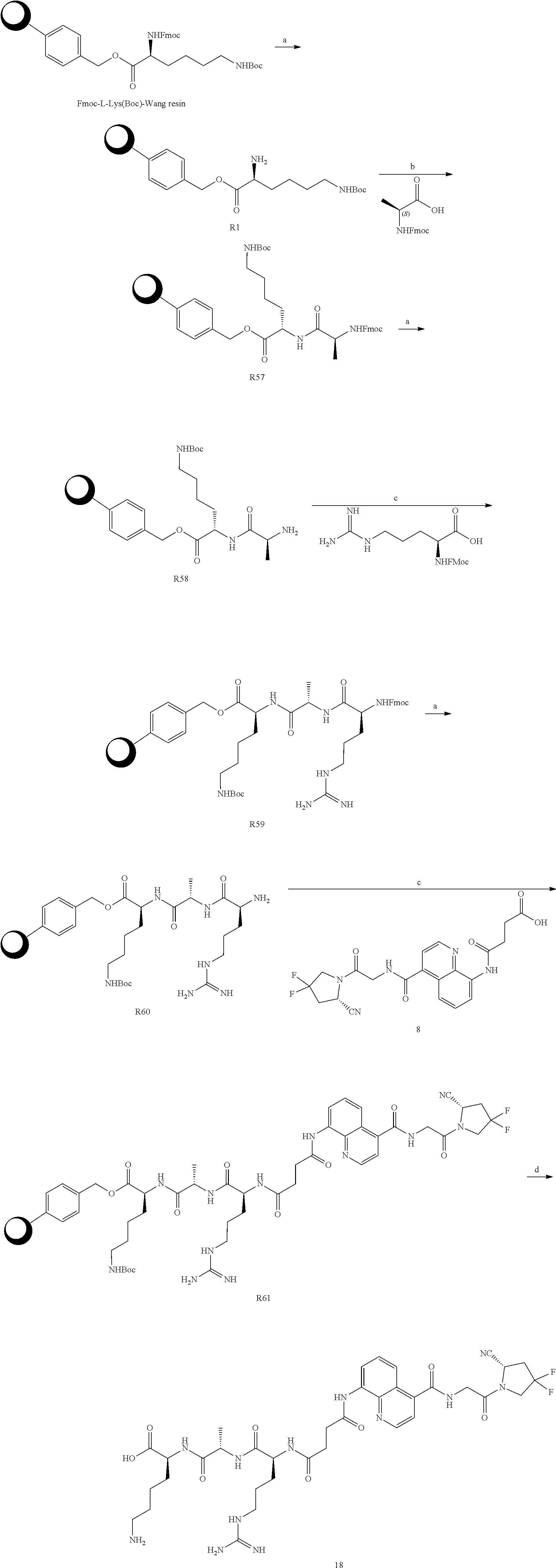

Scheme 16: Reaction scheme for the synthesis of compound 18. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 18. Commercially available Fmoc-L-Lys(Boc)Wang resin (50 mg, 30 gmol) was deprotected and coupled with Fmoc-L-alanine (CAS: 35661-39-3, 0.12 mmol, 37 mg) as reported in the General Procedures (1.2.1 and 1.2.2) obtaining the resin R57. After Fmoc-deprotection (1.2.1), Fmoc-L-Arginine (CAS: 91000-69-0, 0.12 mmol, 48 mg) was coupled using the general procedures 1.2.2 to obtain R59. Then, R59 was Fmoc-deprotected (1.2.1) and coupled (1.2.3) with compound 8 (0.12 mmol, 55 mg) to obtain R61. The product was cleaved from the resin and then purified using the procedure reported in 1.2.5. The final product (18) was afforded with a yield <5%.

### 1.3.15 Preparation of Compound 19

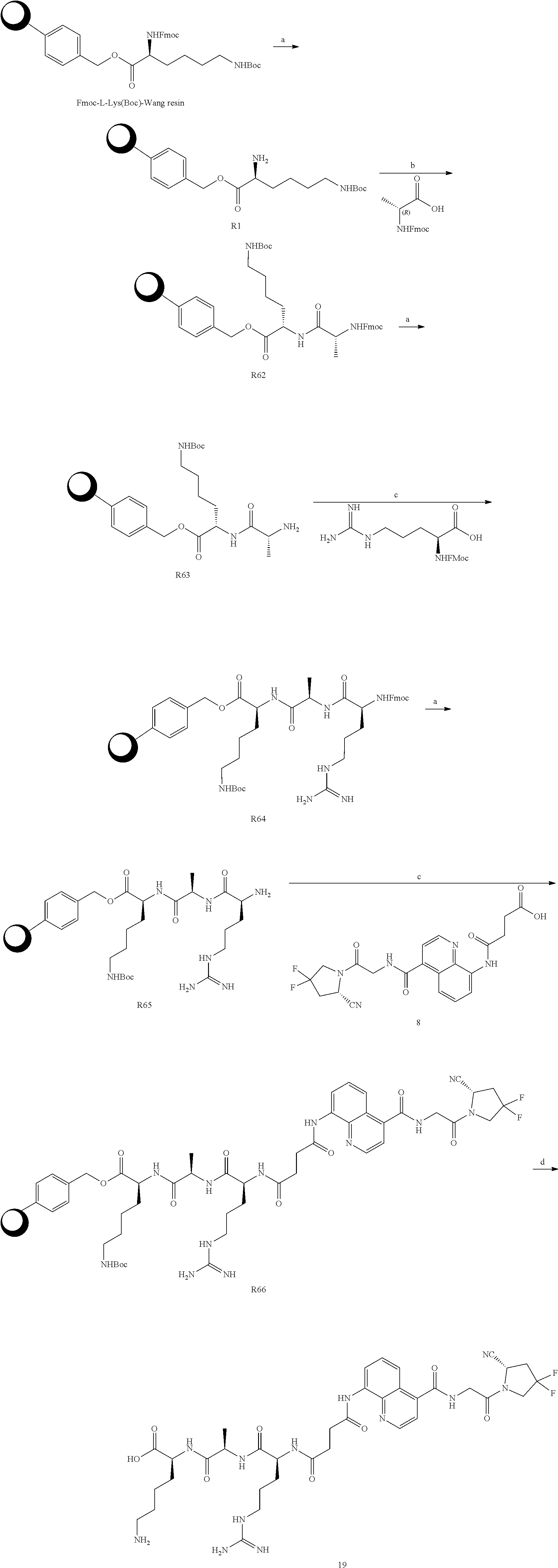

Scheme 17: Reaction scheme for the synthesis of compound 19. Conditions: a) piperidine 20% in N,N-dimethylformamide, 30′ at r.t.; b) 4 eq. of R—CO₂H, 4 eq. of HATU, 4 eq. of DIPEA in N,N-dimethylformamide, 1 h at r.t.; c) 4 eq. of R—CO₂H, 4 eq. of EDC, 4 eq. of DIPEA in N,N-dimethylformamide, 12 h atr.t.; d) TFA 97.5%, TIPS 2.5%, 2 h atr.t.

Synthesis of compound 19. Commercially available Fmoc-L-Lys(Boc)Wang resin (50 mg, 30 gmol) was deprotected and coupled with Fmoc-D-alanine (CAS: 79990-15-1, 0.12 mmol, 37 mg) as reported in the General Procedures (1.2.1 and 1.2.2) obtaining the resin R62. After Fmoc-deprotection (1.2.1), Fmoc-L-Arginine (CAS: 91000-69-0, 0.12 mmol, 48 mg) was coupled using the general procedures 1.2.2 to obtain R64. Then, R64 was Fmoc-deprotected (1.2.1) and coupled (1.2.3) with compound 8 (0.12 mmol, 55 mg) to obtain R66. The product was cleaved from the resin and then purified using the procedure reported in 1.2.5. The final product (19) was afforded with a yield <5%.

### 1.3.16 Preparation of Compound 34

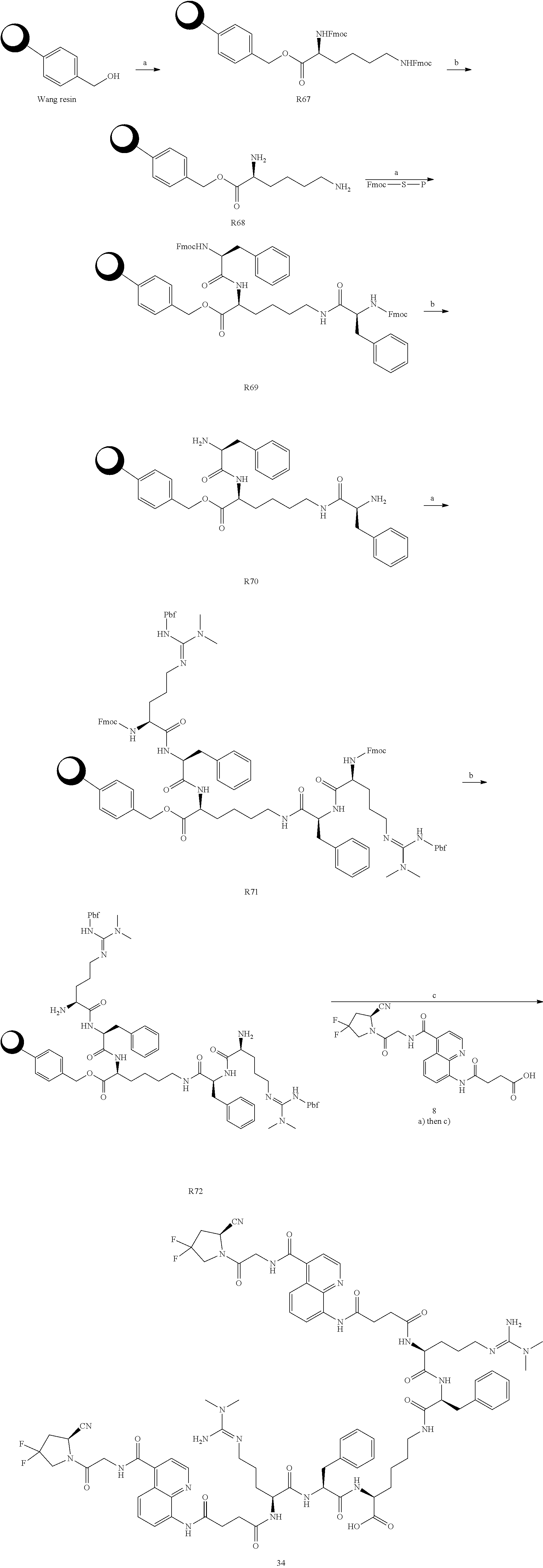

Scheme 18: Reaction scheme for the synthesis of compound 34. Conditions: a) 4 eq. of R—CO₂H, 4 eq. of HATU, 4 eq. of DIPEA in N,N-dimethylfonnamide, 4 h at r.t; b) piperidine 2000 in N,N-dimethylformamnide, 30′ at r.t.; c) TFA 97.500, TIPS 2.50%, 2 h at r.t.

### Synthesis of Compound 34

Commercially available Wang resin (50 mg, 55 gmol) was coupled with Fmoc-Lys(Fmoc)-OH (CAS: 78081-87-5, 220 gmol, 130 mg) as reported in the General Procedures (1.2.2) obtaining the resin R67. After Fmoc-deprotection (1.2.1), compound Fmoc-L-phenylalanine (CAS: 35661-40-6, 220 gmol, 85 mg) was coupled (1.2.2) to obtain R69.

After Fmoc-deprotection (1.2.1), Fmoc-ADMA(Pbf)-OH (220 gmol, 149 mg) was coupled (1.2.2) to obtain R71. After Fmoc-deprotection (1.2.1), compound 8 (220 gmol, 79 mg) was coupled (1.2.2) and then the product was cleaved from the resin and then purified using the procedure reported in 1.2.5. The final product (34) was afforded with a yield <5%.

### 1.3.17 Preparation of Compound 37

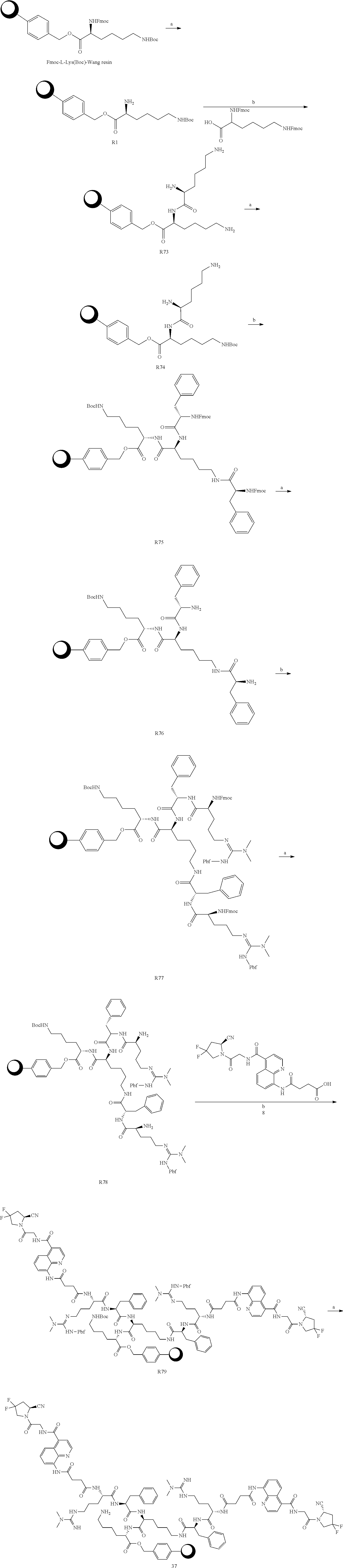

Scheme 19: Reaction scheme for the synthesis of compound 37. Conditions: a) piperidine 20% in N,N-dimethylformamide, 30′ at r.t.; b) 8 eq. of R—CO₂H, 4 eq. of HATU, 4 eq. of DIPEA in N,N-dimethylformamide, 8 h at r.t.; d) TFA 97.5%, TIPS 2.5%, 2 h at r.t . . . .

Synthesis of compound 37. Commercially available Fmoc-L-Lys (Boc) Wang resin (50 mg, 30 μmol) was deprotected and coupled with Fmoc-Lys-(Fmoc)-OH (CAS: 78081-87-5, 0.24 mmol, 142 mg) as reported in the General Procedures (1.2.1 and 1.2.2) obtaining the resin R73. After Fmoc-deprotection (1.2.1), Fmoc-L-Phenylalanine (CAS: 35661-40-6, 0.24 mmol, 93 mg) was coupled using the general procedures 1.2.2 to obtain R75. Then, R75 was Fmoc-deprotected (1.2.1) and coupled (1.2.2) with Fmoc-ADMA (Pbf)-OH (0.24 mmol, 162 mg) to obtain R77. After Fmoc-deprotection (1.2.1), compound 8 (0.24 mmol, 110 mg) was coupled using the general procedures 1.2.1 to obtain R79. The product was cleaved from the resin and then purified using the procedure reported in 1.2.5. The final product (37) was afforded with a yield <5%.

### 1.4 General Remarks for Synthesis in Solution

Liquid-Chromatography/Mass-Spectrometry (LC/MS) spectra presented were recorded on an Agilent 6100 Series Single Quadrupole MS system combined with an Agilent 1200 Series LC, using an InfinityLab Poroshell 120 EC-C18 Column, 2.7 μm, 4.6×50 mm at a flow rate of 0.6 mL min-1, 10% MeCN in 0.1% aq. FA to 100% MeCN in 6 min.

All the chromatograms were registered at λ=260 nm. The purity of the products was assessed by integrating the final chromatogram between 1.1-5.0 minutes. The injection peak (0.0-1.1 minutes) was excluded.

The normal phase column chromatography was performed with normal phase columns in a CombiFlash NEXTGEN 300+ apparatus with RediSepRf silica column.

Reversed-phase column chromatography was performed using BUCHI Sepacore Chromatography line using SepacoreRecord 1.4 as a software with a BUCHI UV Photometer C-635 as a detector.

The columns used were FlashPure Buchi C18 columns 40 μm irregular (24 g).

Reversed-phase high-pressure liquid chromatography (RP-HPLC) was performed on an Agilent 1200 Series RP-HPLC with PDA UV detector, using a Synergi 4 μm, Polar-RP 80 Å 10×150 mm C18 column at a flow rate of 5 mL min⁻¹ with linear gradients of solvents A and B (A=Millipore water with 0.1% TFA, B=MeCN with 0.1% TFA).

### 1.5 Synthesis in Solution

The synthesis of compounds 1, 8, 22, 24, 25, 28, 29, 32, 33, 35, 36, 38, 39 and 41 were performed in solution as described in the procedures below.

### 1.5.1 Preparation of Compound 1

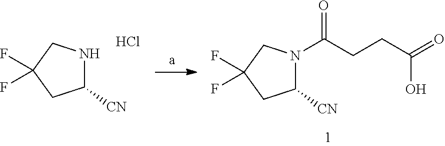 [SMILES: N#C[C@@H]1CC(F)(F)CN1 ; Cl ; N#C[C@@H]1CC(F)(F)CN1C(=O)CCC(=O)O]

Scheme 20: Reaction scheme for the synthesis of compound 1. Conditions: a) 1 eq. (2S)-4,4-difluoropyrrolidine-2-carbonitrile hydrochloride, 10 eq. succinic anhydride, 0.5 eq. DMAP, THF, 1 h at 50° C.

Synthesis of compound 1. Commercially available (2S)-4,4-difluoropyrrolidine-2-carbonitrile hydrochloride (“Gly-ProCN·HCl”, CAS: 869489-04-3, 5.9 mmol, 1.0 g) was dissolved in 5 mL of THF and then DMAP (4-Dimethylaminopyridine; 2.95 mmol, 360 mg) and succinic anhydride (590 mmol, 5.9 g) were added. The solution was then heated at 50° C. for 1 h. The reaction conversion was monitored by LC-MS. The reaction was concentrated under reduced pressure. The crude was poured in a saturated NH₄Cl (20 mL) solution and the product was extracted with EtOAc (3×15 mL). The organic phases were collected, dried over anhydrous Na₂SO₄, filtered and concentrated under pressure. The crude was purified via column chromatography (DCM:DCM/MeOH 8/2 from 0 to 20%) to afford compound 1 with a yield of 80%. 1.5.2 Preparation of compound 8

### 1.5.2.1 Synthesis of Compound 8

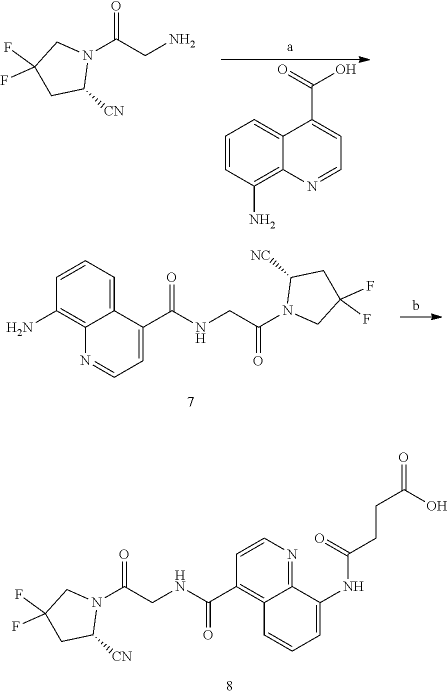

Scheme 21: Reaction scheme for the synthesis of compound 7. Conditions: a) 1 eq. (2S)-1-(2-aminoacetyl)-4,4-difluoropyrrolidine-2-carbonitrile hydrochloride, 1 eq. 8-aminoquinoline-4-carboxylic acid, 1 eq. HATU, 4 eq. DIPEA, DMF/DCM, 30′ from 0° C. to r.t., b) 1 eq. compound 7, 10 eq. succinic anhydride, 0.5 eq. DMAP, THF, 1 h at 50° C.

Synthesis of intermediate 7. Commercially available 2S)-1-(2-aminoacetyl)-4,4-difluoropyrrolidine-2-carbonitrile hydrochloride (CAS: 1448440-51-4, 4.0 mmol, 911 mg) was dissolved in 6 mL of DMF and 14 mL of DCM. 8-aminoquinoline-4-carboxylic acid (CAS: 121689-23-4, 4.0 mmol, 760 mg) and HATU (4.0 mmol, 380 mg) were subsequently added. The mixture was cooled down to 0° C. with an ice bath and then DIPEA (16 mmol, 2.8 mL) was added dropwise to the brown suspension. The reaction was then warmed up to room temperature and stirred for 30′. The reaction conversion was monitored by LC-MS. The reaction was diluted with DCM and washed with a solution of saturated NaCl twice. The organic phase was dried over anhydrous Na₂SO₄, filtered and concentrated under reduced pressure. The crude was then purified via column chromatography (DCM:DCM/MeOH 8/2 from 0 to 20%) to afford compound 7 as a light yellow solid with a yield of 85%.

Synthesis of compound 8. Compound 7 (3.4 mmol, 1.23 g) was dissolved in 12.5 mL of THF and then DMAP (1.7 mmol, 209 mg) and succinic anhydride (34 mmol, 3.42 g) were added. The solution was then heated at 50° C. for 1 h and checked via LC-MS. After completion, the solution was dried under vacuum. The crude was poured in a saturated NH₄Cl (20 mL) solution and the product was extracted with EtOAc (3×15 mL). The organic phases were collected, dried over anhydrous Na₂SO₄, filtered and concentrated under pressure. The crude was then purified via column chromatography (DCM:DCM/MeOH 8/2 from 0 to 20%) to afford compound 8 as a light white solid with a yield of 82%.

### 1.5.2.2 Alternative Synthesis of Compound 8

### Synthesis of 8-(4-(tert-butoxy)-4-oxobutanamido)quinoline-4-carboxylic acid

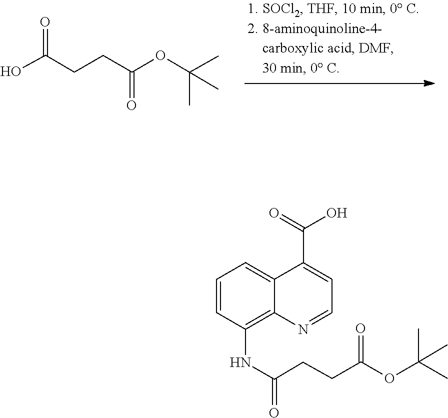 [SMILES: CC(C)(C)OC(=O)CCC(=O)O ; CC(C)(C)OC(=O)CCC(=O)Nc1cccc2c(C(=O)O)ccnc12]

Scheme 22: Reaction scheme for the synthesis of 8-(4-(tert-butoxy)-4-oxobutanamido)quinoline-4-carboxylic acid. 1.2 eq. of 4-(tert-butoxy)-4-oxobutanoic acid, 1.2 eq. SOCl₂, 0° C., THF. Then, 1 eq. 8-aminoquinoline-4-carboxylic acid (in DMF), 30′, 0° C.

To a solution of 4-(tert-butoxy)-4-oxobutanoic acid (57 mg, 0.33 mmol, 1.2 eq) in dry THF (1.5 mL), SOCl₂ (24 μL, 0.33 mmol, 1.2 eq) was slowly added at 0° C. A solution of 8-aminoquinoline-4-carboxylic acid (50 mg, 0.25 mmol, 1 eq) in DMF (500 μL) was then added and the mixture was stirred for 30 min at 0° C. The crude was concentrated under reduced pressure and purified by Reverse Phase MPLC (98:2 to 0:100 AcCN/water+0.1% HCOOH in 45 min). The fractions were collected and lyophilized to afford a white solid (50 mg, 0.15 mmol, 57%).

### Synthesis of tert-butyl (S)-4-((4-((2-(2-cyano-4,4-difluoropyrrolidin-1-yl)-2 oxoethyl)carbamoyl)quinolin-8-yl)amino)-4-oxobutanoate

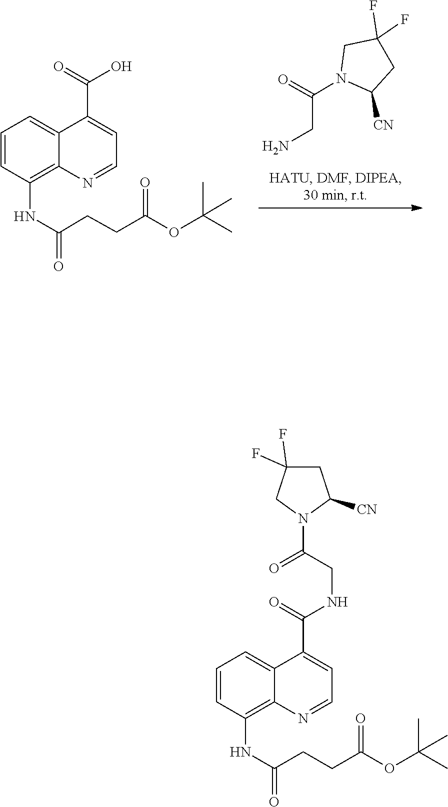 [SMILES: CC(C)(C)OC(=O)CCC(=O)Nc1cccc2c(C(=O)O)ccnc12 ; N#C[C@@H]1CC(F)(F)CN1C(=O)CN ; CC(C)(C)OC(=O)CCC(=O)Nc1cccc2c(C(=O)NCC(=O)N3CC(F)(F)C[C@H]3C#N)ccnc12]

Scheme 23: Reaction scheme for the synthesis of tert-butyl (S)-4-((4-((2-(2-cyano-4,4-difluoropyrrolidin-1-yl)-2 oxoethyl)carbamoyl)quinolin-8-yl)amino)-4-oxobutanoate. 1 eq. 8-(4-(tert-butoxy)-4-oxobutanamido)quinoline-4-carboxylic acid, 1 eq. S)-1-(2-aminoacetyl)-4,4-difluoropyrrolidine-2-carbonitrile hydrochloride, 1 eq. HATU, 4 eq. DIPEA, DMF, 15′, 25° C. 8-(4-(tert-butoxy)-4-oxobutanamido)quinoline-4-carboxylic acid (50 mg, 0.15 mmol, 1 eq), (S)-1-(2-aminoacetyl)-4,4-difluoropyrrolidine-2-carbonitrile hydrochloride (33 mg, 0.15 mmol, 1 eq) and HATU (55 g, 0.15 mmol, 1 eq) were suspended in 2.0 mL of DMF. DIPEA (100 μL, 0.6 mmol, 4 eq) was added dropwise and the reaction was stirred for 15 minutes. The crude was diluted with DCM, washed with water, dried over anhydrous Na₂SO₄, filtered and the solvent evaporated under vacuum. The crude was concentrated under reduced pressure and purified by Reverse Phase MPLC (98:2 to 0:100 AcCN/water+0.1% HCOOH in 45 min). The fractions were collected and lyophilized to afford a white solid (40 mg, 0.077 mmol, 52%).

### Synthesis of compound 8 ((S)-4-((4-((2-(2-cyano-4,4-difluoropyrrolidin-1-yl)-2-oxoethyl)carbamoyl)quinolin-8-yl)amino)-4-oxobutanoic acid)

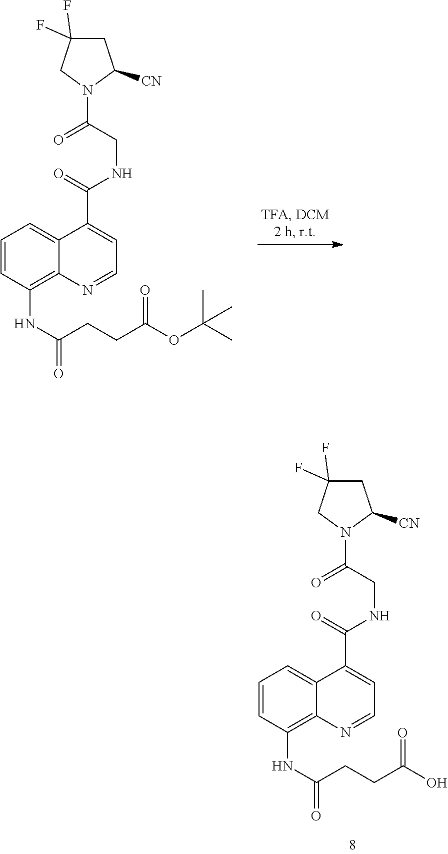 [SMILES: CC(C)(C)OC(=O)CCC(=O)Nc1cccc2c(C(=O)NCC(=O)N3CC(F)(F)C[C@H]3C#N)ccnc12 ; N#C[C@@H]1CC(F)(F)CN1C(=O)CNC(=O)c1ccnc2c(NC(=O)CCC(=O)O)cccc12]

Scheme 24: Reaction scheme for the synthesis of compound 8. 1 eq. tert-butyl (S)-4-((4-((2-(2-cyano-4,4-difluoropyrrolidin-1-yl)-2-oxoethyl)carbamoyl)quinolin-8-yl)amino)-4-oxobutanoate, 1:1 mix DCM:TFA (50 eq), 2 h, from 0° C. to 25° C.

tert-butyl (S)-4-((4-((2-(2-cyano-4,4-difluoropyrrolidin-1-yl)-2-oxoethyl)carbamoyl)quinolin-8-yl)amino)-4-oxobutanoate (40 mg, 0.077 mmol, 1 eq) was dissolved in DCM (285 μL) and TFA (285 μL, 3.85 mmol, 50 eq) was added dropwise at 0° C. The mixture was stirred for 2 hours and then concentrated under reduced pressure. The crude was purified by Reverse Phase MPLC (98:2 to 0:100 AcCN/water+0.1% HCOOH in 45 min). The fractions were collected and lyophilized to afford a white solid (25 mg, 0.054 mmol, 71%).

### 1.5.3 Preparation of Compound 22

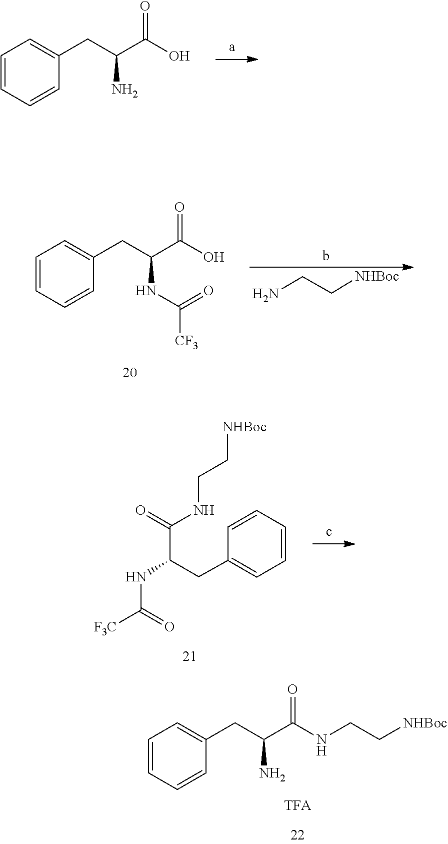 [SMILES: N[C@@H](Cc1ccccc1)C(=O)O ; O=C(O)[C@H](Cc1ccccc1)NC(=O)C(F)(F)F ; CC(C)(C)OC(=O)NCCN ; CC(C)(C)OC(=O)NCCNC(=O)[C@H](Cc1ccccc1)NC(=O)C(F)(F)F ; CC(C)(C)OC(=O)NCCNC(=O)[C@@H](N)Cc1ccccc1]

Scheme 25: Reaction scheme for the synthesis of compound 22. Conditions: a) 3 eq. CF₃CO₂Et, 3 eq. TEA, anhydrous MeOH, 1 h, r.t.; b) 2 eq. N-Boc-ethylenediamine, 1.2 eq. HATU, 3 eq. DIPEA, DMF:DCM=1:1, 1 h, r.t.; c) 20% NH3 in MeOH:H₂O=1:1, 12 h, reflux.

### Synthesis of Intermediate 20.

Commercially available L-Phenylalanine (CAS: 63-91-2, 6.0 mmol, 1.0 g) was dissolved in 5 mL of anhydrous MeOH and then ethyl trifluoroacetate (CAS: 383-63-1, 12.0 mmol, 720 μL) and triethylamine (12.0 mmol, 1.7 mL) were added. After 30′ at r.t., a second aliquot of ethyl trifluoroacetate (CAS: 383-63-1, 6.0 mmol, 360 μL) and triethylamine (6.0 mmol, 850 μL) were added and the resulting solution was stirred for additional 30′ at r.t. The mixture was then evaporated and dissolved in 10 mL of DCM. The crude product was washed with a solution of aqueous HCl 0.1M (3×10 mL) and the organic phase was dried over anhydrous Na2SO4, filtered and concentrated under reduced pressure to afford compound 20 as a white solid with a yield of 95%.

### Synthesis of Intermediate 21.

Compound 20 (3.8 mmol, 1.0 g) was dissolved in 5 mL of DMF and 5 mL of DCM and N-Boc-ethylenediamine (CAS: 57260-73-8, 7.6 mmol, 1.2 mL), HATU (4.6 mmol, 1.7 g) and DIPEA (11.4 mmol, 2.0 mL) were added. The reaction was kept for 1 h at r.t. The reaction conversion was monitored by LC-MS. The reaction was concentrated under vacuum, diluted with 30 mL of DCM and washed with water (3×10 mL), NaHCO₃ aqueous saturated solution (2×10 mL) and KHSO₄ aqueous saturated solution (2×10 mL). The organic layer was collected, dried over anhydrous Na₂SO₄, filtered and concentrated under pressure to afford compound 21 as a white solid.

The crude product was dissolved in mQ Millipore water:acetonitrile=1:1 mixture (1 mL) and purified by RP-HPLC with acetonitrile, 0.1% trifluoroacetic acid (buffer B): H₂O, 0.1% trifluoroacetic acid (buffer A) as an eluent (5% B for 2 minutes; from 5% to 40% B in 8 minutes, from 40% to 60% B in 3 minutes, from 60% to 100% B in 2 minutes and 100% B for 2 mins). The compound 22 was obtained with a yield of 60%.

### Synthesis of Compound 22.

Compound 21 (1.2 mmol, 500 mg) was dissolved in a 5 mL of a 20% solution of NH₃ in MeOH:water=1:1. The reaction was refluxed for 12 h. The reaction conversion was monitored by LC-MS. After completion, the solution was concentrated and extracted with DCM (3×10 mL). The organic phases were collected, dried over anhydrous Na₂SO₄, filtered and concentrated under pressure. The crude was then dissolved in mQ Millipore water:acetonitrile=1:1 mixture (1 mL) and purified via RP-HPLC with the gradient described in General procedures 1.2.5 to obtain compound 22 with an yield of 54%.

### 1.5.4 Preparation of Compound 24

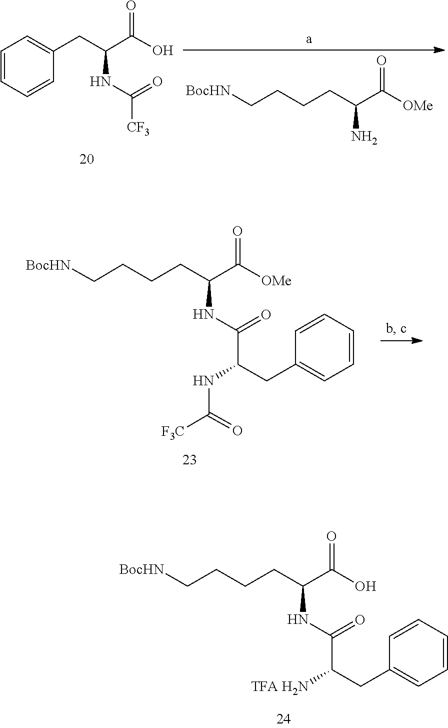

Scheme 26: Reaction scheme for the synthesis of compound 24. Conditions: a) 2 eq. H-Lys(Boc)OMe, 1.2 eq. HATU, 3 eq. DIPEA, DMF:DCM=1:1, 1 h, r.t.; b) NaOH 1M in MeOH:H₂O=1:1, 1 h, r.t.; c) 20% NH₃ in MeOH:H₂O=1:1, 12 h, reflux.

### Synthesis of Intermediate 23.

Compound 20 (3.8 mmol, 1.0 g) was dissolved in 5 mL of DMF and 5 mL of DCM and it was reacted with Lys(Boc)OMe (CAS: 3017-32-1, 7.6 mmol, 2.0 g), HATU (4.6 mmol, 1.7 g) and DIPEA (11.4 mmol, 2.0 mL) for 1 h at r.t. The reaction conversion was monitored by LC-MS. The crude product was dissolved in DCM (30 mL) and washed with water (3×10 mL), NaHCO₃ aqueous saturated solution (2×10 mL) and KHSO₄ aqueous saturated solution (2×10 mL). The organic layer was dried over anhydrous Na₂SO₄, filtered and reduced under pressure to afford compound 23.

### Synthesis of Compound 24.

Compound 23 (0.99 mmol, 500 mg) was dissolved in 5 mL of MeOH and 1 M NaOH aqueous solution (5 mL) was added. The resulting solution was stirred for 1 h at r.t. The reaction conversion was monitored by LC-MS. The crude product was concentrated and reacted with 5 mL of a 20% solution of NH₃ in MeOH:water=1:1. After completion, the solution was concentrated, diluted with 5 mL of water and extracted with DCM (3×10 mL). The organic phases were collected, dried over anhydrous Na₂SO₄, filtered and reduced under pressure. The crude was then dissolved in mQ Millipore water:acetonitrile=1:1 mixture (1 mL) and then purified via RP-HPLC with the same gradient described in General procedures 1.2.5 to obtain compound 24.

### 1.5.5 Preparation of Compound 25

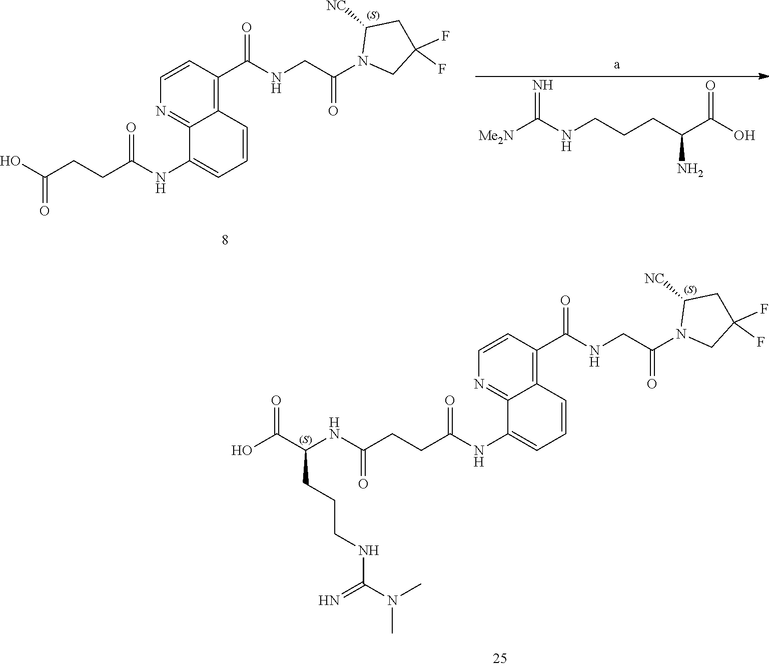

Scheme 27: Reaction scheme for the synthesis of compound 25. Conditions: a) 2 eq. HATU, 4 eq. DIPEA, 2 eq. NG,NG-Dimethylarginine, DMF, 1 h, r.t.

Synthesis of compound 25. Compound 8 (0.43 mmol, 200 mg) was dissolved in 5 mL of DMF and it was reacted with NG,NG-Dimethylarginine (CAS: 30315-93-6, 0.86 mmol, 174 mg), HATU (0.86 mmol, 327 mg) and DIPEA (0.86 mmol, 150 μL) for 1 h at r.t. After completion, the solution was directly purified in reverse phase column chromatography with a gradient of 100% water+0.1% formic acid for 5′, then from 0% to 100% of acetonitrile+0.1% of formic acid in 40′ and then 100% acetonitrile+0.1% of formic acid for 10′, to obtain compound 25 with an yield of 16%.

### 1.5.6 Preparation of Compound 28

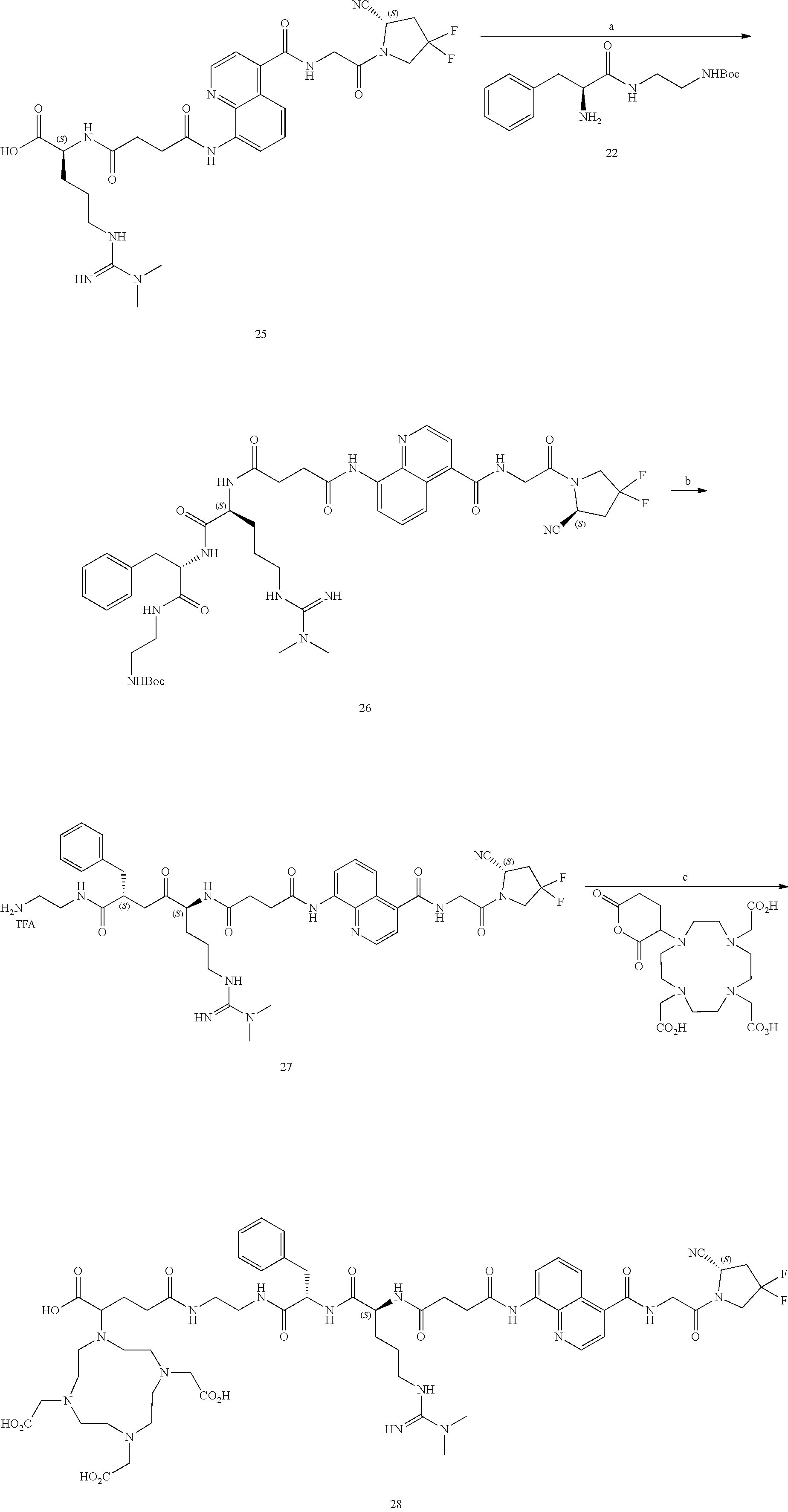

Scheme 28: Reaction scheme for the synthesis of compound 28. Conditions: a) 6 eq. EDC, 2 eq. HOAt, 4 eq. DIPEA, 2.5 eq. Compound 22, DMF, 3 h, r.t.; b) TFA, 1 h, from 0° C. to r.t.; c) 2 eq Py, 0.5 eq. DMAP, 1.5 eq. DOTA-GA anhydride; THF, 1 h, r.t.

Synthesis of intermediate 26. Compound 25 (0.05 mmol, 35 mg) was dissolved in 2 mL of DMF and compound 22 (0.075 mmol, 23 mg), EDC (0.10 mmol, 8.2 μL), HOAt (0.10 mmol, 14 mg) and DIPEA (0.2 mmol, 35 μL) were added. The resulting solution was stirred for 1 h at r.t. Then, to the reaction EDC (0.10 mmol, 8.2 μL) was added. After 5′ of stirring, an additional aliquot of compound 22 was added (0,015 mmol, 7.7 mg). The reaction was left stirring for 1 hour at r.t. and the last addition of EDC (0.10 mmol, 8.2 μL) and compound 22 (0,015 mmol, 7.7 mg) was performed. The reaction was stirred for an additional hour. The crude product was directly purified via RP-HPLC with the gradient described in General procedures 1.2.5 to obtain compound 26 with an yield of 85%.

Synthesis of intermediate 27. Compound 26 (0.025 mmol, 23 mg) was cooled down at 0° C. and dissolved in 200 μL of 99% TFA. The reaction was stirred for 1 h at r.t. The reaction conversion was monitored by LC-MS. After completion, the mixture was diluted in 50 mL of water:acetonitrile=1:1 and lyophilised obtaining compound 27 with an yield of 90%.

### Synthesis of Compound 28.

Compound 27 (0.025 mmol, 24 mg) was dissolved in 500 μL of THF. To the solution, pyridine (0.05 mmol, 4 μL), 4-dimethylaminopyridine (0.013 mmol, 1.5 mg), and DOTA-GA anhydride (0.038 mmol, 17 mg) were added and the mixture was stirred for 1 h at r.t. After completion, the solution was dried, dissolved in mQ Millipore water:acetonitrile=1:1 mixture (1 mL) and purified via RP-HPLC with the gradient described in General procedures 1.2.5 to obtain compound 28.

### 1.5.7 Preparation of Compound 29

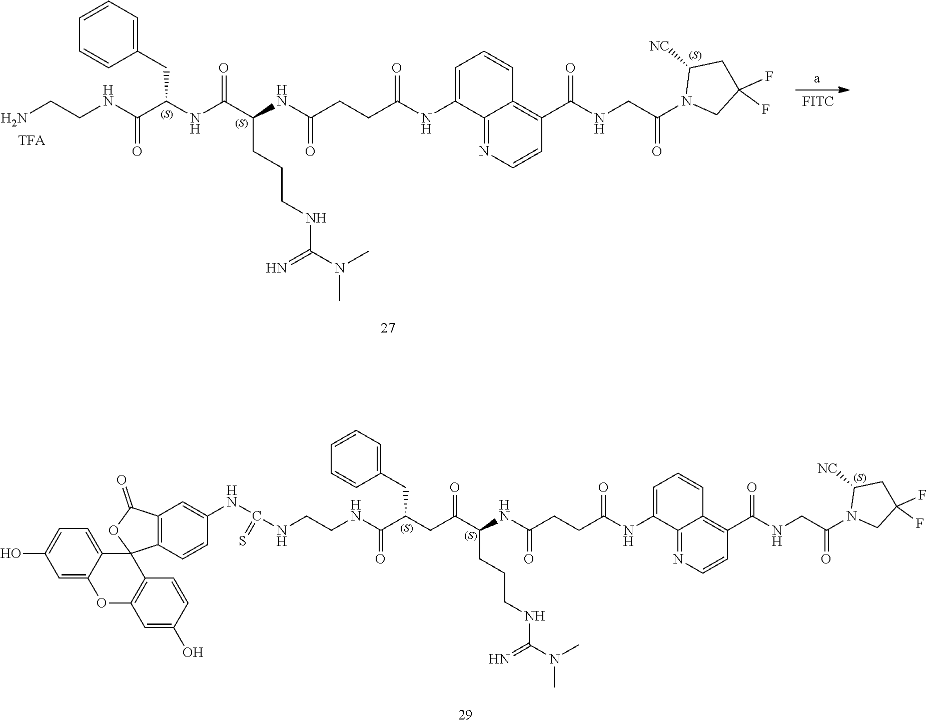

Scheme 29: Reaction scheme for the synthesis of compound 29. Conditions: a) 1.5 eq. FITC, 10 eq. TEA, DMSO, 1 h, r.t.

### Synthesis of Compound 29.

Compound 27 (0.025 mmol, 24 mg) was dissolved in 500 μL of DMSO and triethylamine (0.25 mmol, 35 μL) and fluorescein 5-isothiocyanate (5-FITC, 0.0375 mmol, 15 mg) were added. The reaction was left stirring for 1 h at r.t. After completion, the crude was directly purified via RP-HPLC with the gradient described in General procedures 1.2.5 to obtain compound 29.

### 1.5.8 Preparation of Compound 32

### 1.5.8.1 Synthesis of Compound 32

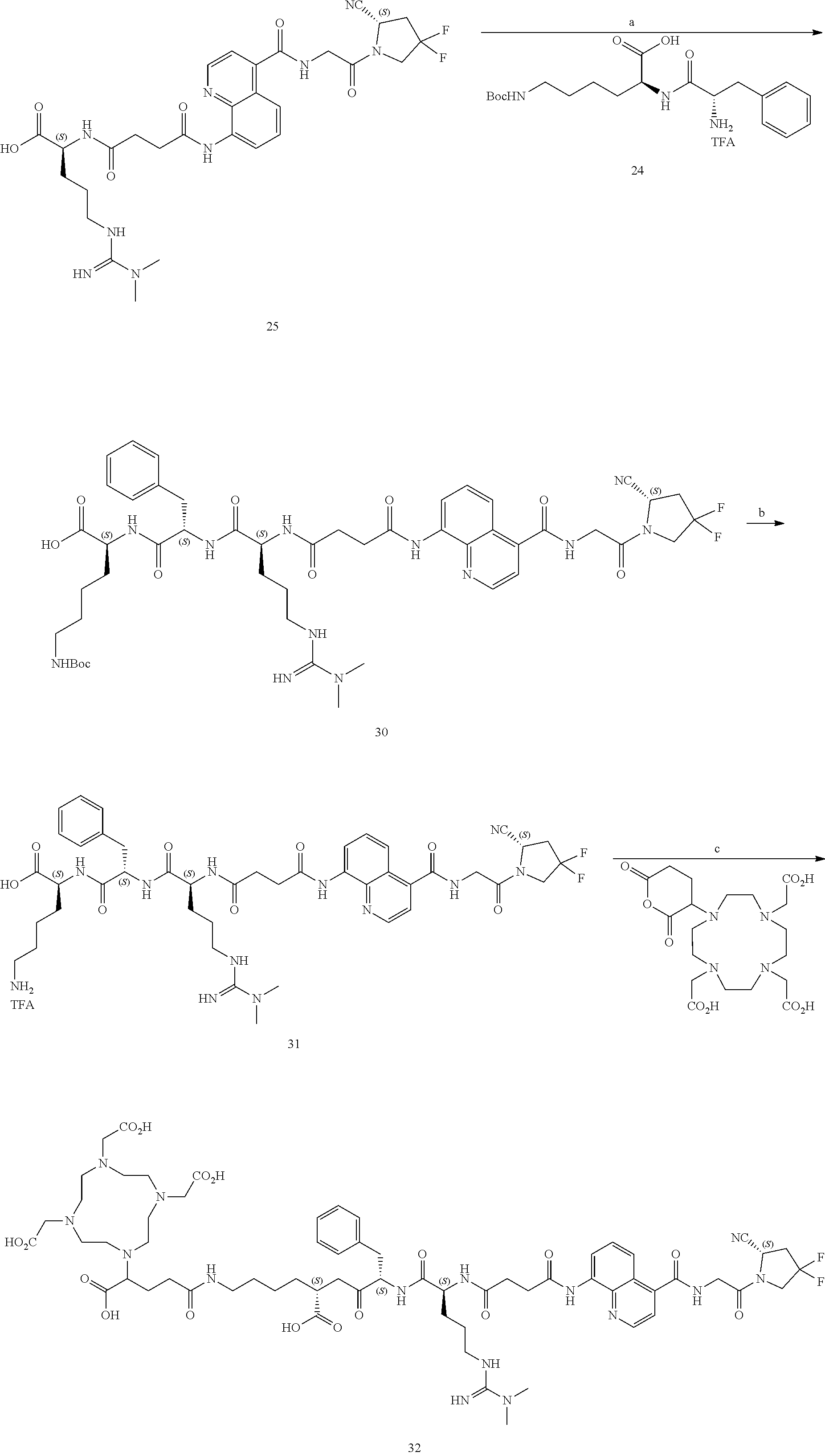

Scheme 30: Reaction scheme for the synthesis of compound 32. Conditions: a) 2 eq. EDC, 2 eq. HOAt, 4 eq. DIPEA, 2.5 eq. Compound 24, DMF, 3 h, r.t.; b) TFA, 1 h, r.t.; c) 2 eq Py, 0.5 eq. DMAP, 1.5 eq. DOTA-GA anhydride; THF, 1 h, r.t.

### Synthesis of Intermediate 30.

Compound 25 (0.05 mmol, 35 mg) was dissolved in 2 mL of DMF, compound 24 (0.075 mmol, 38 mg), EDC (0.10 mmol, 8.2 μL), HOAt (0.10 mmol, 14 mg) and DIPEA (0.2 mmol, 35 μL) were added. The resulting solution was stirred for 1 h at r.t. The crude product was purified via RP-HPLC with the same gradient described in General procedures 1.2.5 to obtain compound 30.

### Synthesis of Intermediate 31.

Compound 30 (0.025 mmol, 25 mg) was cooled down at 0° C. and dissolved in 200 μL of 99% TFA. The reaction was stirred for 1 h at r.t. The reaction conversion was monitored by LC-MS. After completion, the mixture was diluted in 50 mL of water:acetonitrile=1:1 and lyophilised obtaining compound 31.

### Synthesis of Compound 32.

Compound 31 (0.025 mmol, 26 mg) was dissolved in 500 μL of THF. To the solution, pyridine (0.05 mmol, 4 μL), 4-dimethylaminopyridine (0.013 mmol, 1.5 mg), and DOTA-GA anhydride (0.038 mmol, 17 mg) were added and the mixture was stirred for 1 h at r.t. After completion, the solution was concentrated and the crude was dissolved in mQ Millipore water:acetonitrile=1:1 mixture (1 mL) and purified via RP-HPLC with the gradient described in General procedures 1.2.5 to obtain compound 32.

### 1.5.8.2 Alternative Synthesis of Compound 32

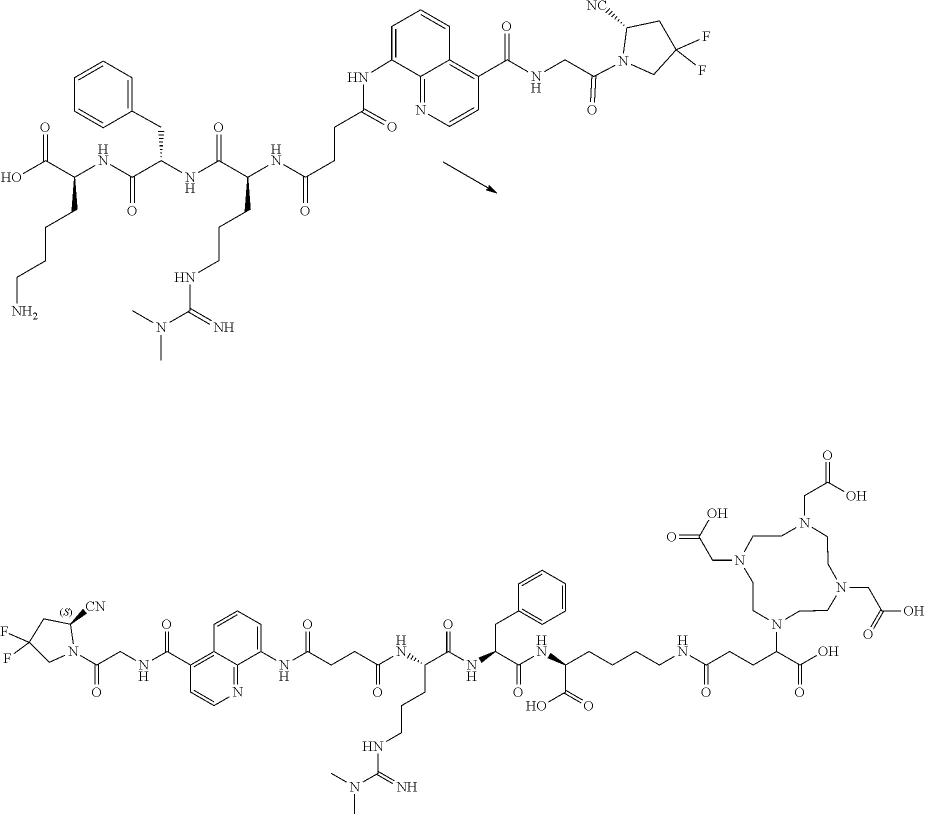

Scheme 31: 2 eq Py, 0.5 eq. DMAP, 1.5 eq. DOTA-GA anhydride; THF, 1 h, r.t.

Synthesis of compound 32. Compound 11 (0.025 mmol, 23 mg) was dissolved in 500 μL of DMSO. To the solution, pyridine (0.05 mmol, 4 μL), 4-dimethylaminopyridine (0.013 mmol, 1.5 mg), and DOTA-GA anhydride (0.038 mmol, 17 mg) were added and the mixture was stirred for 1 h at r.t. After completion, the solution was dried, dissolved in mQ Millipore water:acetonitrile=1:1 mixture (1 mL) and purified via RP-HPL C with the gradient described in General procedures 1.2.5 to obtain compound 32.

### 1.5.9 Preparation of Compound 33

### 1.5.9.1 Synthesis of Compound 33

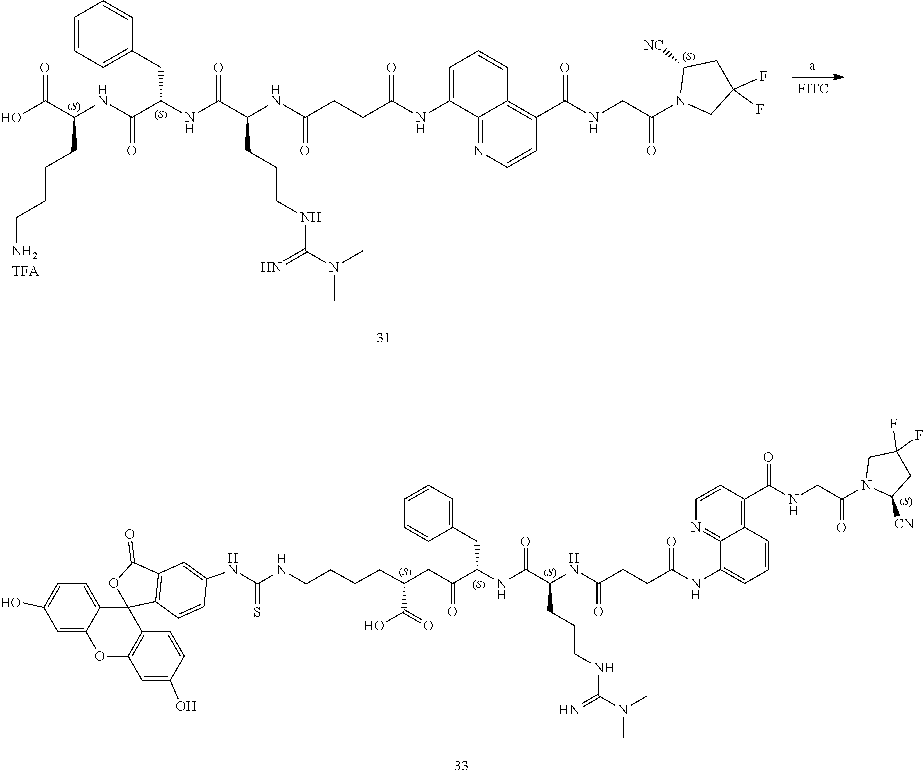

Scheme 32: Reaction scheme for the synthesis of compound 33. Conditions: a) 1.5 eq. FITC, 10 eq. TEA, DMSO, 1 h, r.t.

### Synthesis of Compound 33.

Compound 31 (0.025 mmol, 26 mg) was dissolved in 500 μL of DMSO and triethylamine (0.25 mmol, 35 μL) and fluorescein 5-isothiocyanate (5-FITC, 0.0375 mmol, 15 mg) were added. The reaction was left stirring for 1 h at r.t. After completion, the crude was directly purified via RP-HPLC with the gradient described in General procedures 1.2.5 to obtain compound 33.

### 1.5.9.1 Alternative Synthesis of Compound 33

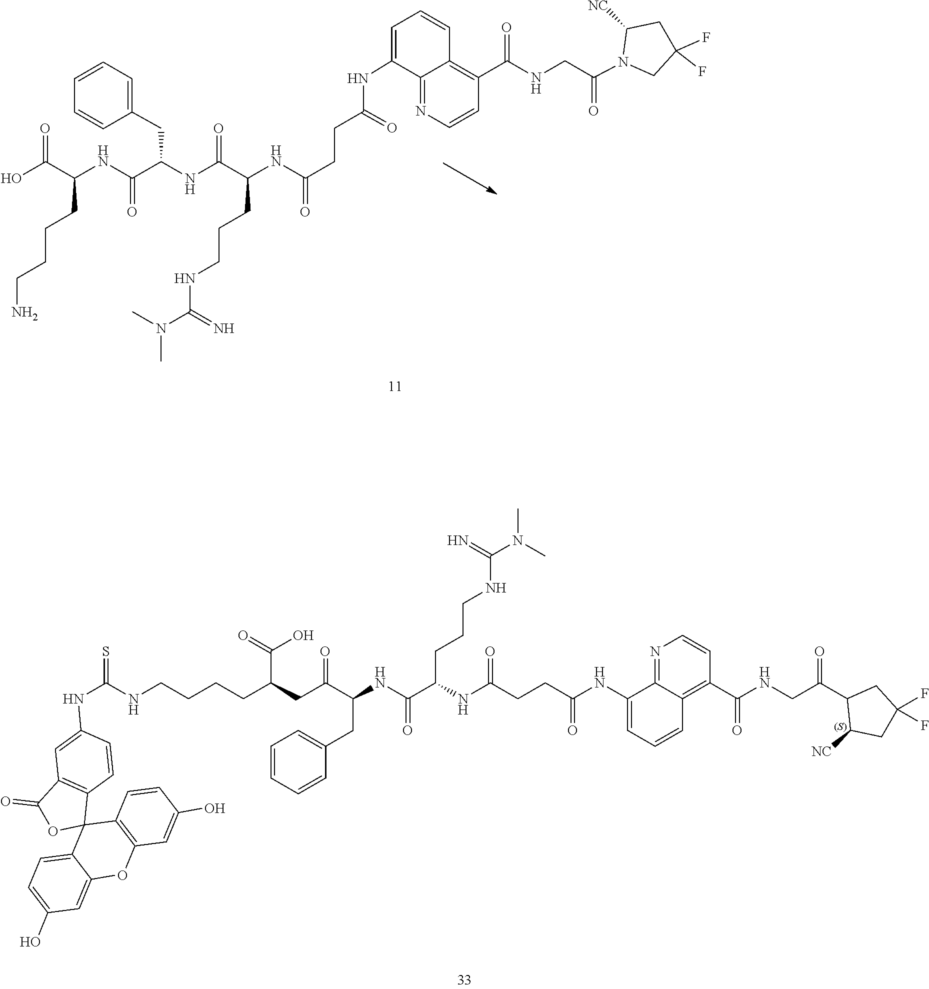

Scheme 33: Reaction scheme for the synthesis of compound 33 (ESV6-11-Lys-Fluo). Conditions: a) 1.5 eq. FITC, 10 eq. TEA, DMSO, 1 h, r.t.

### Alternative Synthesis of Compound 33

Compound 11 (0.025 mmol, 23 mg) was dissolved in 500 μL of DMSO and triethylamine (0.25 mmol, 35 μL) and fluorescein 5-isothiocyanate (5-FITC, 0.0375 mmol, 15 mg) were added. The reaction was left stirring for 1 h at r.t. After completion, the crude was directly purified via RP-HPLC with the gradient described in General procedures 1.2.5 to obtain compound 33.

### 1.5.10 Preparation of Compound 35

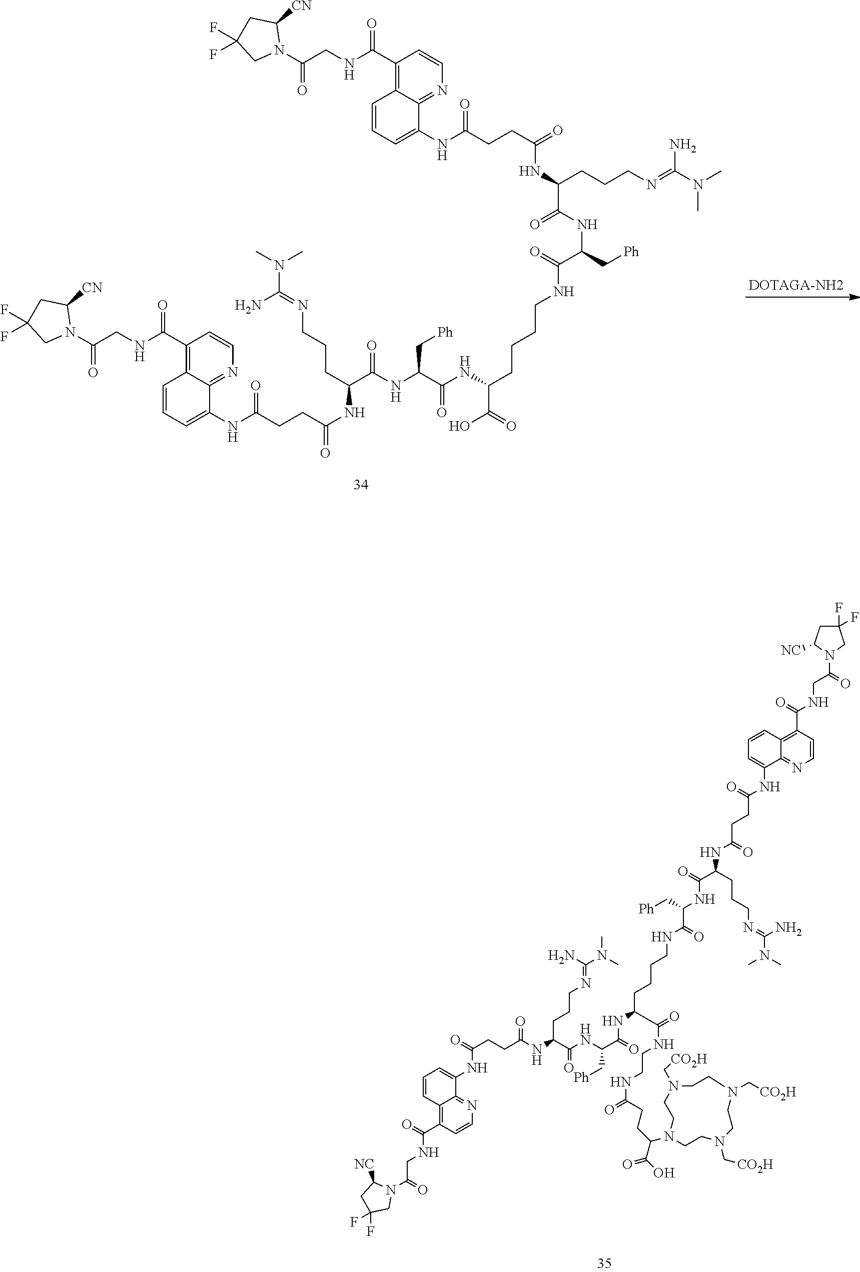

Scheme 34: Reaction scheme for the synthesis of compound 35. Conditions: 2 eq. of N-Hydroxysuccinimide, 1.2 eq. of HATU, 5 eq. of DIPEA (0.15 mL, 0.88 mmol, 5 eq.) in dry in N,N-dimethylformamide, 30′, r.t. Then 2 eq. of (R)-DOTA-GA-NH2, 12 h, r.t.

### Synthesis of Compound 35

N-Hydroxysuccinimide (0.1 mg, 1.2 gmol, 2 eq.), HATU (0.3 mg, 0.72 gmol, 1.2 eq.) and DIPEA (521 μL, 3 gmol, 5 eq.) were added to a solution of compound 34 (1 mg, 0.6 gmol, 1 eq.) in dry DMF (0.2 mL). The reaction solution was stirred for 30 minutes at room temperature, then (R)-DOTA-GA-NH₂ (0.6 mg, 1.2 gmol, 2 eq.) was added. The resulting mixture was stirred vigorously overnight, then diluted with milliQ water (0.5 mL) and purified via RP-HPLC (90:10 to 0:100 ACN/water+0.1% TFA in 12 min). The desired fractions were collected and lyophilized to afford a white solid.

### 1.5.11 Preparation of Compound 36

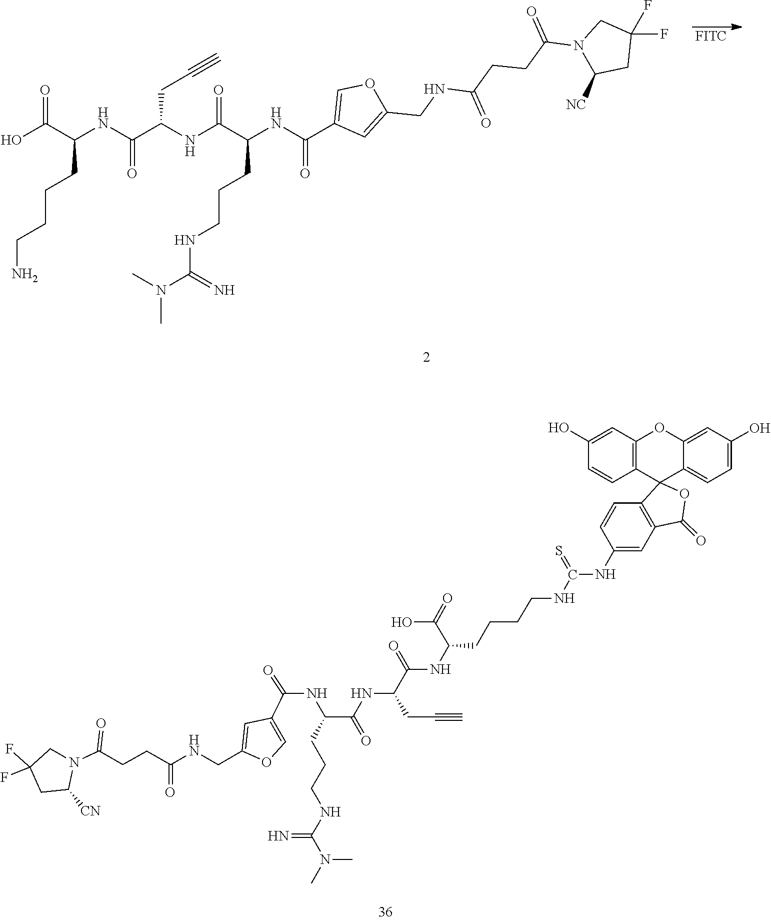

Scheme 35: reaction scheme for the synthesis of compound 36. 1 eq. compound 2, 1.5 eq. FITC, DMSO, 1 h, 24° C.

### Synthesis of Compound 36.

Compound 2 (0.013 mmol, 10 mg) was dissolved in 250 μL of DMSO and triethylamine (0.13 mmol, 18 μL) and fluorescein 5-isothiocyanate (5-FITC, 0.019 mmol, 8 mg) were added. The reaction was left stirring for 1 h at 24° C. After completion, the crude was purified via RP-HPLC (2.3) affording compound 36 that was directly dissolved in DMSO. The concentration was calculated by measuring the sample absorbance at A=498 nm (ε=73′000 cm⁻¹M⁻¹) and further diluted with PB to a final concentration of 2 nM. m/z calculated for C₅₅H₅₉F₂N₁₁O₁₃S: [M+2H]²⁺576.70641, detected (HR-MS ESI+) 576.70455.

### 1.5.12 Preparation of Compound 38

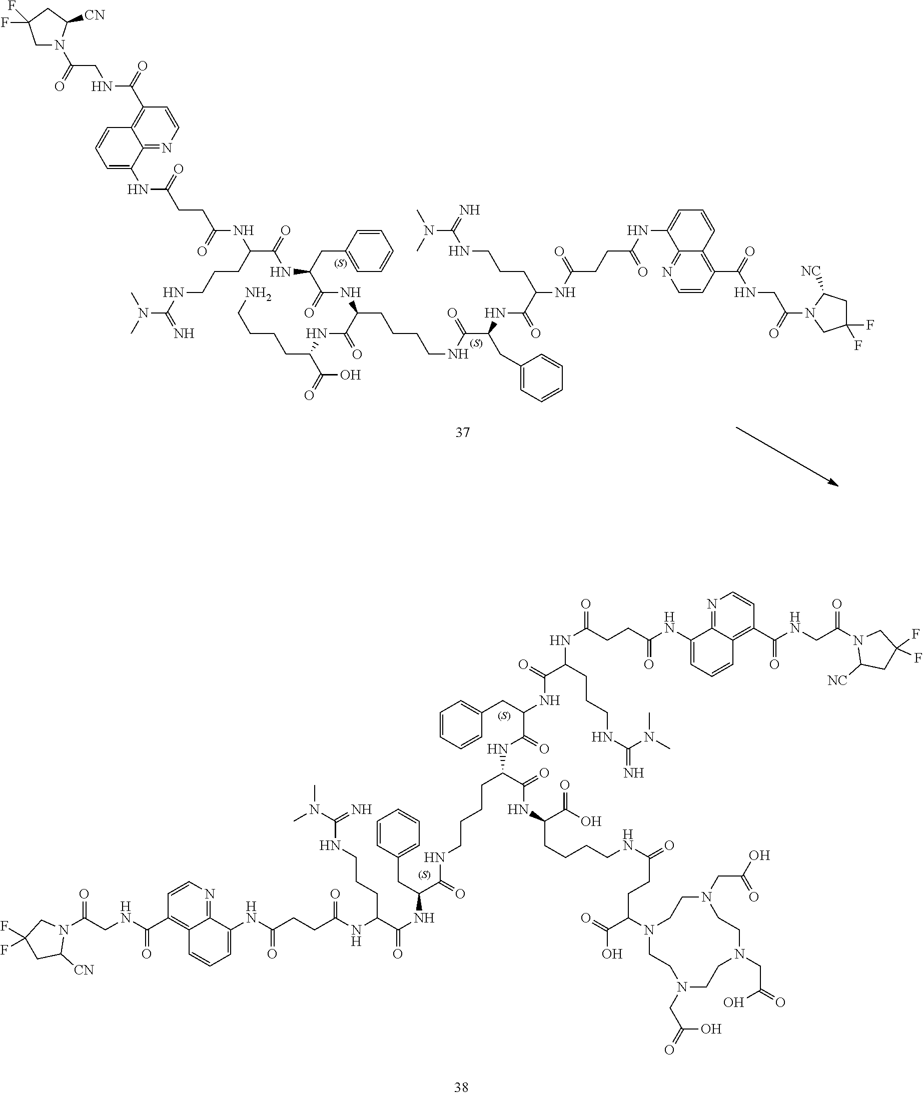

Scheme 36: 2 eq Py, 0.5 eq. DMAP, 1.5 eq. DOTA-GA anhydride, THF, 1 h, r.t.

### Synthesis of Compound 38.

Compound 37 (0.025 mmol, 46 mg) was dissolved in 500 μL of DMSO. To the solution, pyridine (0.05 mmol, 4 μL), 4-dimethylaminopyridine (0.013 mmol, 1.5 mg), and DOTA-GA anhydride (0.038 mmol, 17 mg) were added and the mixture was stirred for 1 h at r.t. After completion, the solution was dried, dissolved in mQ Millipore water:acetonitrile=1:1 mixture (1 mL) and purified via RP-HPLC with the gradient described in General procedures 1.2.5 to obtain compound 38.

### 1.5.13 Preparation of Compound 39

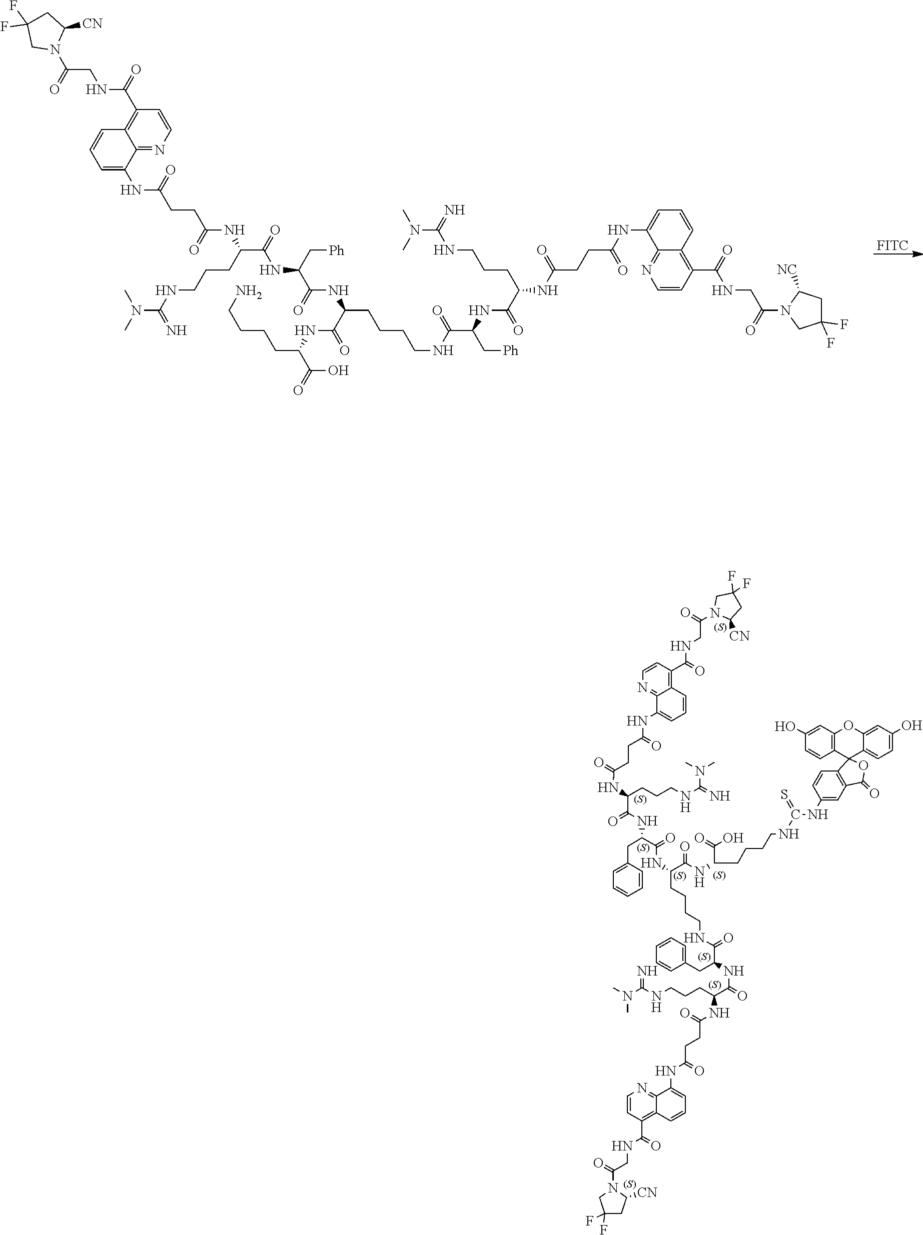

Scheme 37: Reaction scheme for the synthesis of compound 39. Conditions: a) 1.5 eq. FITC, 10 eq. TEA, DMSO, 1 h, r.t.

### Synthesis of Compound 39

Compound 37 (0.025 mmol, 46 mg) was dissolved in 500 μL of DMSO and triethylamine (0.25 mmol, 35 μL) and fluorescein 5-isothiocyanate (5-FITC, 0.0375 mmol, 15 mg) were added. The reaction was left stirring for 1 h at r.t. After completion, the crude was directly purified via RP-HPLC with the gradient described in General procedures 1.2.5 to obtain compound 39.

### 1.5.14 Preparation of Compound 41 (ESV6-11-Gly-Pro-Exatecan)

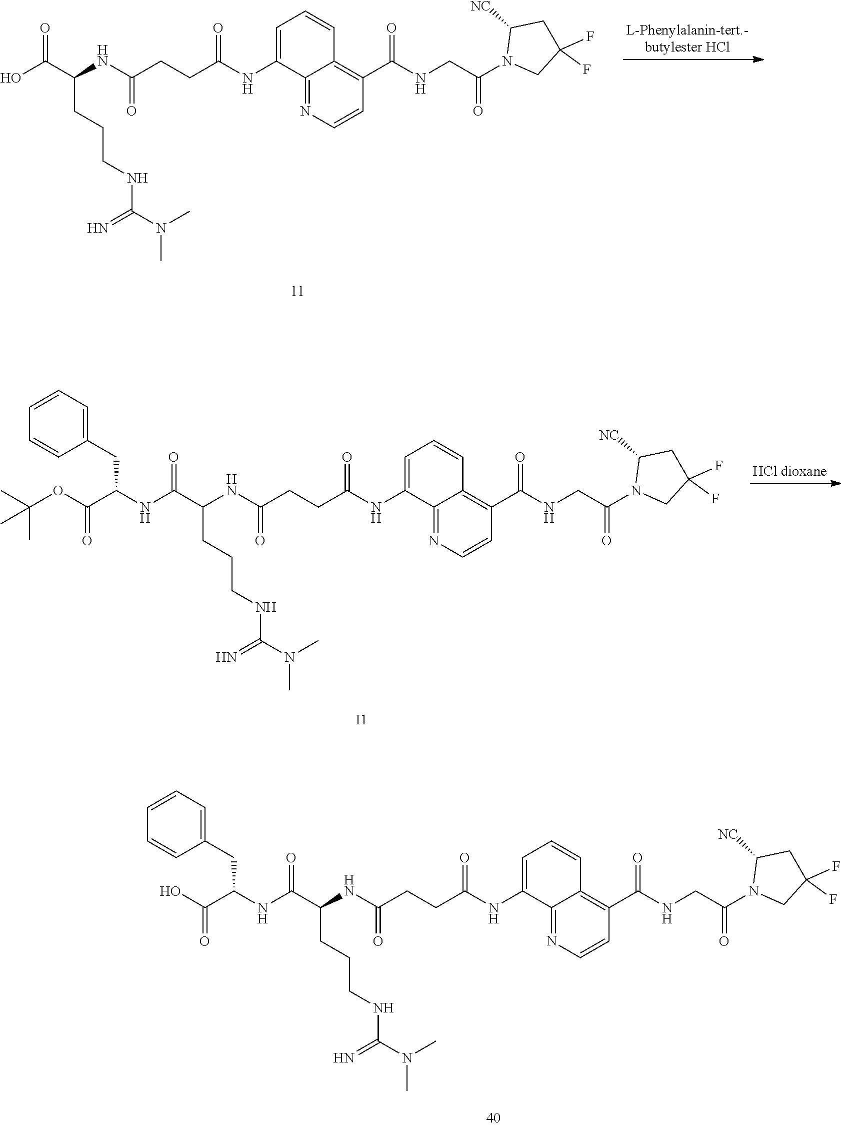 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)O ; CN(C)C(=N)NCCCC(NC(=O)CCC(=O)Nc1cccc2c(C(=O)NCC(=O)N3CC(F)(F)C[C@H]3C#N)ccnc12)C(=O)N[C@@H](Cc1ccccc1)C(=O)OC(C)(C)C ; "I1" ; CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)O]

Scheme 38: reaction scheme for the synthesis of compound 40 (ESV6-11-COOH). 1. 1 eq. R—COOH, 1.2 eq. L-Phe-tBu HCL, 1.1 eq.HATU, 3 eq. DIPEA, DMF, 3 h, 24° C. 2. 1 eq. Boc-derivative, 10 eq. HCl 4M in dioxane, 1 h, 24° C.

Synthesis of compound 40. The free acid molecule (compound 11) was reacted with L-Phe-tBu HCl (1.2 eq) in the presence of HATU (1.1 eq) and DIPEA (3 eq) in DMF for 3 hours at room temperature. The crude product (I1) was purified by RP-chromatography (Büchi Sepacore equipped with C18 40 μM irregular column using a gradient of 98:2 to 0:100 in 30 min water/ACN+0.1% HCOOH).

Compound I1 was then reacted in dioxane with HCl dioxane 4M (10 eq) for 1 hour and purified by RP-HPLC (gradient of 90:10 to 0:100 in 14 min water/ACN+0.1% TFA) to afford compound 40.

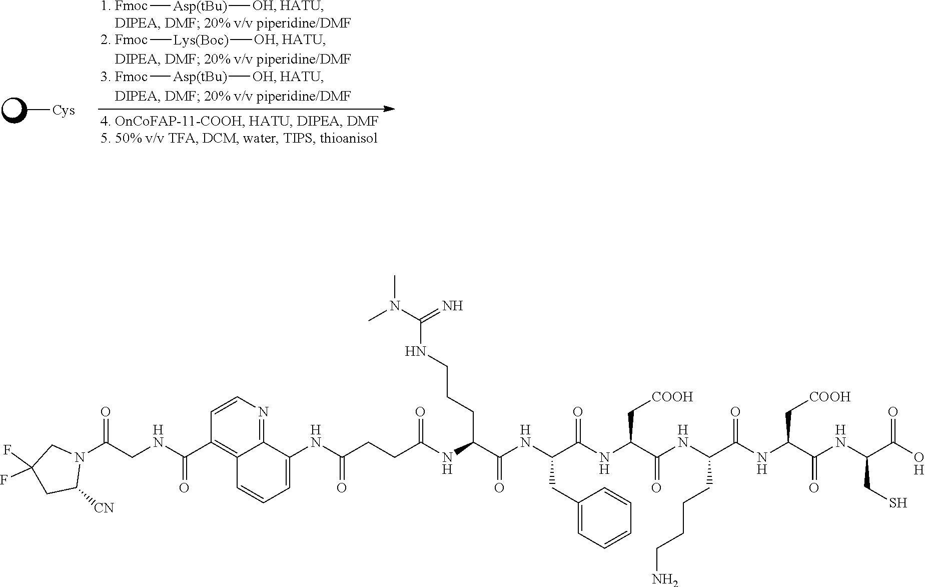

Scheme 39: reaction scheme for the synthesis of ESV6-11-Asp-Lys-Asp. 1. 2 eq Fmoc-Asp(tBu)-OH, 2 eq. HATU, 4 eq. DIPEA, DMF, 1 h, 24° C., then piperidine 20% in N,N-dimethylformamide, 30′, 24° C. 2. 2 eq Fmoc-Lys(Boc)-OH, 2 eq. HATU, 4 eq. DIPEA, DMF, 1 h, 24° C., then piperidine 20% in N,N-dimethylformamide, 30′, 24° C. 3. 2 eq Fmoc-Asp(tBu)-OH, 2 eq. HATU, 4 eq. DIPEA, DMF, 1 h, 24° C., then piperidine 20% in N,N-dimethylformamide, 30′, 24° C. 4. 2 eq. ESV6-11-COOH, 2 eq. HATU, 4 eq. DIPEA, DMF, 1 h, 24° C., then piperidine 20% in N,N-dimethylformamide, 30′, 24° C. 5. TFA 50%, TIPS 10%, DCM 35%. water 3%. thioanisol 2%, 2 h at 24° C.

### Synthesis of ESV6-11-Asp-Lys-Asp.

Commercially available pre-loaded Fmoc-Cys(Trt) on Tentagel resin (RAPP Polymere, 250 mg, 0.15 mmol loading) was swollen in DMF. The peptide was extended with Fmoc-Asp(tBu)-OH, FmocLys(NHBoc)-OH and Fmoc-Asp(tBu)-OH in the indicated order. For this purpose, the Fmoc protected amino acid (2.0 eq), HATU (2.0 eq) and DIPEA (4.0 eq) were dissolved in DMF (5 mL) and reacted with the resin for 1 h under gentle agitation. After washing with DMF (6×1 min×5 mL) the Fmoc group was removed with 20% piperidine in DMF (1×1 min×5 mL and 2×10 min×5 mL). Deprotection steps were followed by wash steps with DMF (6×1 min×5 mL) prior to coupling with the next amino acid. Compound 40 (ESV6-11-COOH) (387 mg, 0.30 mmol, 2 eq.) was coupled as described. The peptide was cleaved from the resin with a cleavage cocktail composed by TFA (50%), Thioanisole (2%), Triisopropylsilane (10%), mQ water (3%) and DCM (35%). The crude product was purified by RP-chromatography (gradient of 98:2 to 0:100 in 30 min water/ACN+0.1% HCOOH). The desired fractions were collected and lyophilized to afford ESV6-11-Asp-Lys-Asp as a solid.

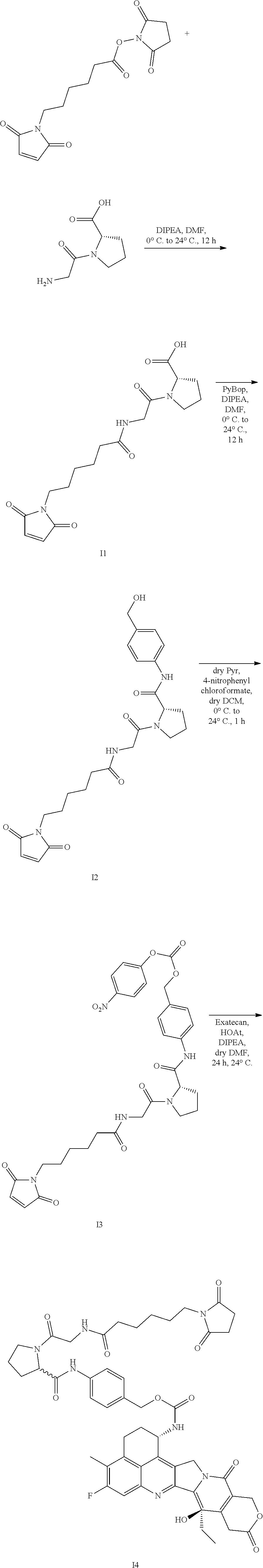

Scheme 40: reaction scheme for the synthesis of I4 (MC-Gly-Pro-Exatecan): I1: 1 eq. MC-OSu, 1 eq. Glycyl-L-proline, 2 eq. DIPEA, from 0° to 24° C., 12 h. I2: 1.5 eq. PyBop, 2 eq. DIEPA, DCM, 0° C. to 24° C., 12 h. I3: 1.5 eq dry pyridine, 2.2 eq. 4-nitrophenyl chloroformate, dry DCM, from 0° C. to 24° C., 1 h. I4: 1.2 eq. Exatecan, 0.5 eq. HOAt, 2 eq. DIPEA, dry DMF, 24° C., 24 h.

### Synthesis Intermediate I1

MC-OSu (1 eq.) and Glycyl-L-proline (1 eq.) were suspended in DMF and the solution was cooled down at 0° C. in an ice bath. DIPEA (2 eq.) was added dropwise to the cooled mixture and stirred at room temperature overnight. Then purified via RP-chromatography (gradient of 98:2 to 0:100 in 30 min water/ACN+0.1% HCOOH) to afford a colorless foam.

### Synthesis Intermediate I2

I1 (1 eq.), 4-aminobenzyl alcohol (1.5 eq.) and PyBOP (1.5 eq.) were dissolved in DCM (0.3 mmol/mL) and cooled to 0° C. DIPEA (2 eq.) was added dropwise to the reaction mixture and stirred at room temperature overnight. The solution was transferred to a separatory funnel, diluted with DCM and washed with a saturated aqueous solution of NaHCO₃, HCl 1M and brine. The organic phase was dried over anhydrous Na₂SO₄, filtered and evaporated under reduced pressure to afford a yellow oil. The crude was purified via flash column chromatography (95:5 DCM/MeOH) to afford a white solid.

### Synthesis Intermediate I3

I2 (1 eq.) was dissolved in a mixture of dry pyridine (1.5 eq.) and dry DCM (0.5 mmol/mL). The solution was cooled at 0° C., then 4-nitrophenyl chloroformate (2.2 eq.) was added as a solution in dry DCM. The reaction was stirred at room temperature for 1 h then directly purified via column chromatography (EtOAc/hexane 1:1) to afford a white solid.

### Synthesis Intermediate I4

I3 (1 eq.), Exatecan (1.2 eq.), HOAt (0.5 eq.) were dissolved in dry DMF (0.1 mmol/mL) and DIPEA (2 eq.) was added. The reaction was stirred for 24 h, then purified via RP-HPLC (gradient of 90:10 to 0:100 in 14 min water/ACN+0.1% TFA). The desired fraction were collected and lyophilized to afford a white solid.

### Synthesis of Compound 41

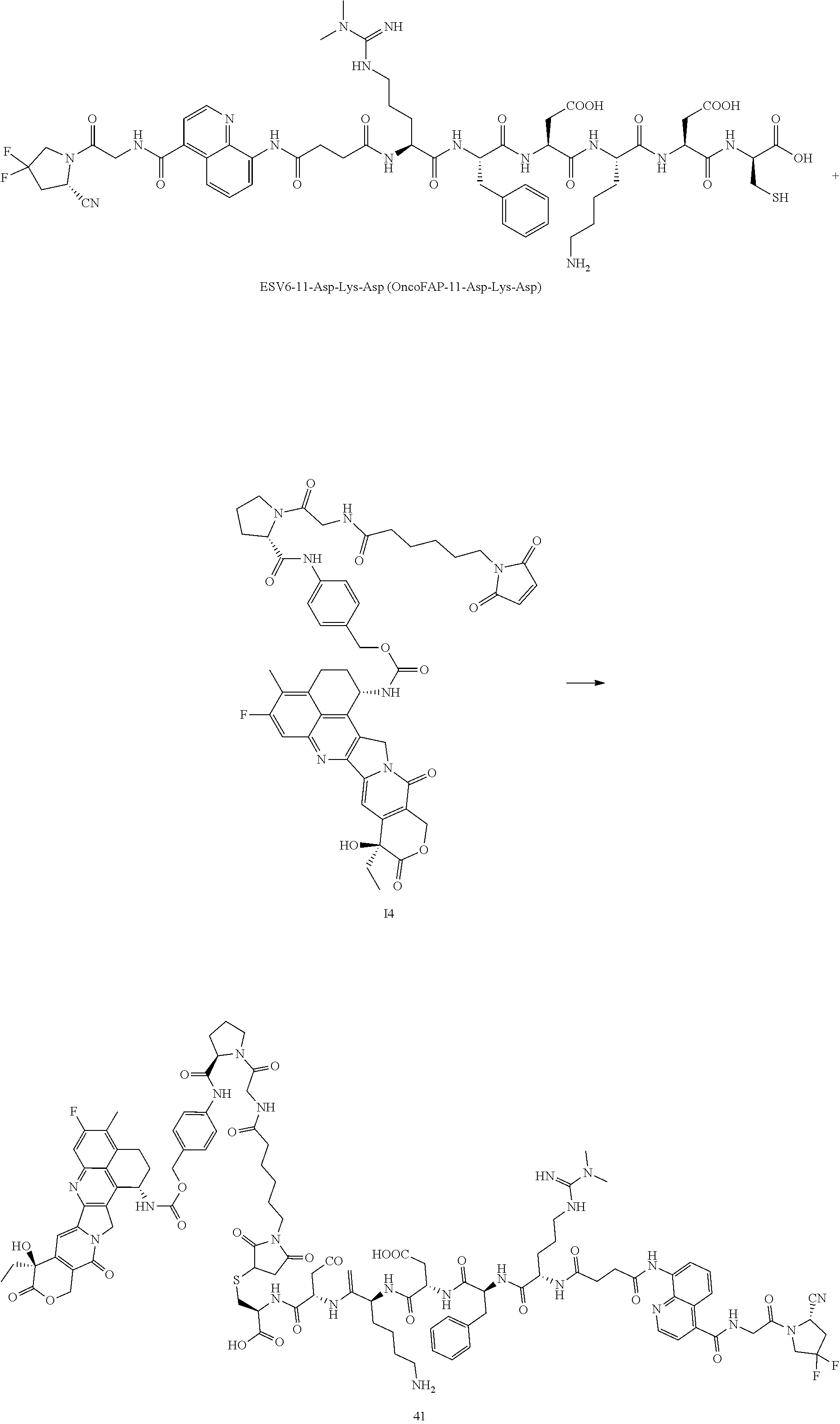

Scheme 41: reaction scheme for the synthesis of 41. 1 eq. I4, 1.2 eq. ESV6-11-Asp-Lys-Asp, 1:1 PBS/DMF, f NaHCO₃, 1 h, 24° C.

I4 (1 eq.) and ESV6-11-Asp-Lys-Asp (1.2 eq.) were dissolved in a 1:1 mixture of PBS (pH=7.4) and DMF (0.01 mmol/mL). Few drops of NaHCO₃ aqueous were added and the reaction was stirred for 1 hour at 24° C. Then the mixture was purified via RP-HIPLC (gradient of 90:10 to 0:100 in 14 min water/ACN+0.100 TFA). The product was collected and lyophilized affording compound 41.

### 2. Inhibition Assays

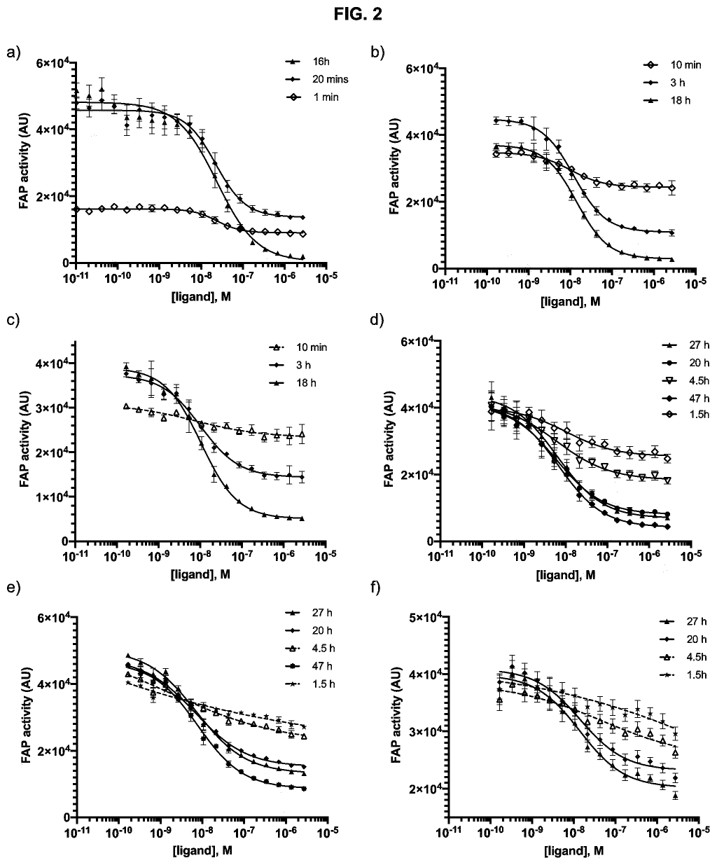

Inhibition assay of FAP. The compounds were precisely weighed (range between 20-1000 pg) on a micro-scale (HuberLab, Sartorius QUINTIX35-1S) and dissolved in DMSO to prepare stock solutions at 1 mM. Stock solutions were further diluted with FAP activation buffer (50 mM Tris, 100 mM NaCl, 1 mM EDTA, pH=7.4) to a final DMSO concentration of 10% v/v. 10 μL of each inhibitor were serially diluted in a 384 well plate (grenier-bio one, PS, F-bottom, Black, non-binding) with 10 μL of 10% DMSO FAP buffer (1:1 dilution). FAP (human and murine) was diluted to the desired concentration with FAP activation buffer and added to the serial dilution of inhibitor (10 μL for each well). Each protein-inhibitor solution was incubated for 30 minutes at room temperature. Each measurement was performed with an enzyme concentration (FAP) lower than the expected IC50. A 60 μM solution of substrate (Z-Gly-Pro-AMC) in FAP activation buffer (<1% DMSO) was added to the plate (10 μL for each well). The reaction was incubated from 1 minute to 48 hours (see FIG. 2) at room temperature (22° C.-25° C.). The emission was read at 465 nm (excitation wavelength 360 nm) for 40 μs by Tecan Spark (s.n. 1808004082). The following controls were added to each row: positive control (no inhibitor control, 10 μL of 10% DMSO FAP buffer+10 μL of FAP solution+10 μL of substrate solution), negative control (no protein control, 10 μL of 10% DMSO FAP buffer+10 μL of FAP activation buffer+10 μL of substrate solution).

Normalization of values: The “% of FAP activity” can be calculated with the following formula:

$$\% of FAP activity = \frac{RAW value ( AU ) - no protein RAW value ( AU )}{no inhibitor value ( AU ) - no protein RAW value ( AU )} \cdot 100 \%$$

### Brief Description of the Results

FIG. 2: Inhibition assay performed overtime with compound 8 (ESV6-succinic) at 22.6° C. with different concentrations human FAP: a) [hFAP]=2.5 nM, b) [hFAP]=625 pM, c) [hFAP]=312 pM, d) [hFAP]=156 pM, e) [hFAP]=78 pM, f) [hFAP]=39 pM. The concentration of substrate (Z-Gly-Pro-AMC) was kept constant=20 μM. Dashed lines corresponds to sets of data with unstable fitting. The fitted curves yielded IC₅₀ values ranging between 21 nM to 6 nM. Error bars display SEM.

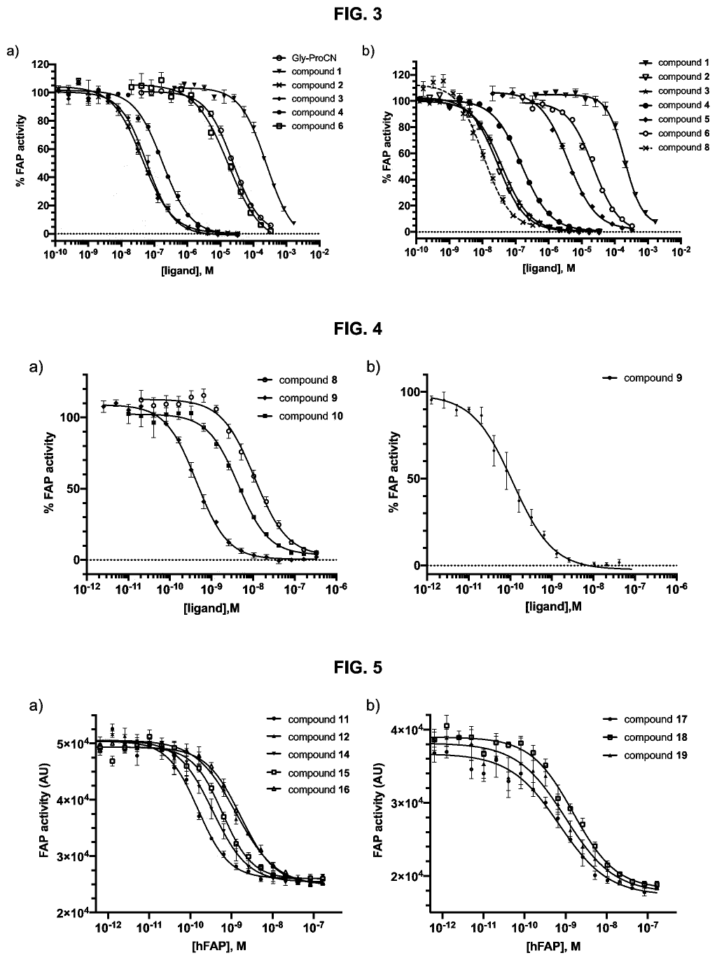

FIG. 3: Inhibition assay performed with compounds 1, 2, 3, 4, 5, 6, 8 against a) 66 pM human FAP and b) 66 pM murine FAP. The concentration of substrate (Z-Gly-Pro-AMC) was kept constant=20 μM. The assay was incubated for 48 hours at 22.6° C. The fitted curves yielded IC₅₀ values reported in Tables 1 and 2. Error bars display SEM.

FIG. 4: a) Inhibition assay performed with compounds 8, 9 and 10 against 66 pM human FAP. The assay was incubated for 17 hours at 25° C. b) Inhibition assay performed with compound 9 against 66 pM murine FAP. The assay was incubated for 17 hours at 25° C. In both assays the concentration of substrate (Z-Gly-Pro-AMC) was kept constant=20 μM. The fitted curves yielded IC₅₀ values reported in Tables 1 and 2. Error bars display SEM.

FIG. 5: Inhibition assay performed with compounds 11, 12, 14-16 (a) and 17-19 (b).

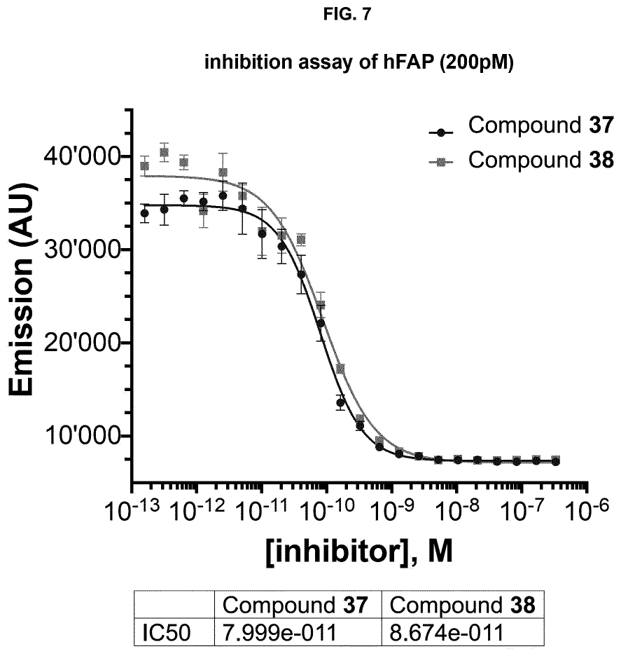

FIG. 7: Inhibition assay performed with compounds 37 and 38 (Bi-ESV6-11 and Bi-ESV6-11-DOTAGA) against 66 pM human FAP. The assay was incubated for 24 hours at 25° C. In both assays the concentration of substrate (Z-Gly-Pro-AMC) was kept constant=20 μM. The fitted curves yielded IC₅₀ values reported in Table 2. Error bars display SEM.

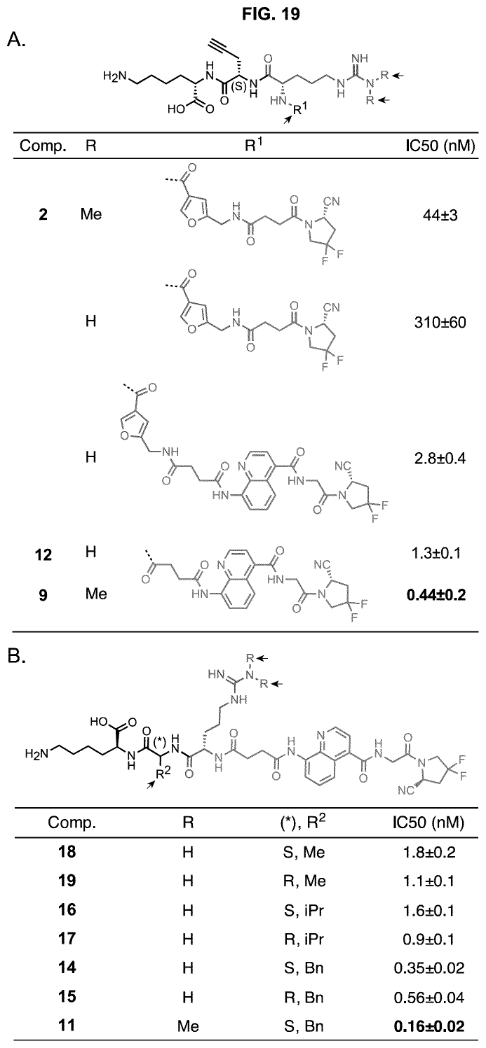

FIG. 19. A: Chemical structures and corresponding IC50_{(hFAP)} values of compounds according to the present invention. B: Chemical structures and corresponding IC50 values of compounds where the propargyl moiety at position AA² was replaced by different groups (Me, iPr, Bn) using both R and S stereoisomers. The sites of modification have been highlighted with arrows. Each measurement was performed with a FAP concentration lower than the expected IC50. Depending on FAP concentration, the fluorescence was measured between 10 minutes and 48 h (at room temperature) after the addition of the fluorogenic substrate (Z-Gly-Pro-AMC). IC50 values and standard errors were calculated using “[Inhibitor] vs. response—Variable slope (four parameters)” fitting (Graphpad Prism). FIG. 19A shows the series of analogues synthesized starting from compound 2, comprising the S stereoisomer of propargylglycine. The particularly advantageous effect of the two methyl groups (R=Me) is indicated by results upon substitution of N^{G}, N^{G}-dimethyl-L-arginine with L-arginine to the compound having R=H. Further investigation of structure-activity relationships (SAR) was performed using the natural arginine, which facilitated the discrimination of inhibition constants in the sub-nanomolar range. By introducing the quinoline-based moiety, a single-digit nanomolar inhibitor was obtained. The furan-based moiety was subsequently omitted without a loss of potency (compound 12), and the substitution of L-arginine by N^{G},N^{G}-dimethyl-L-arginine led to compound 9 with an inhibitory potency in the sub-nanomolar range. FIG. 19B shows the contribution to FAP inhibition activity of the terminal alkyne group of propargylglycine was investigated as indicated in Both stereoisomers (S and R) were tested with alanine (compounds 18 and 19), valine (compounds 16 and 17) and phenylalanine (compounds 14 and 15) in combination with an L-arginine building block (R=H). The replacement of propargylglycine with S-phenylalanine improved potency of the lead compound of additional ˜2-folds (compound 14). Reintroduction of N^{G},N^{G}-dimethyl-L-arginine (A122) yielded compound 11, with a FAP inhibitory potency in the picomolar range against human FAP that was used as lead compound for further in vitro and in vivo characterization.

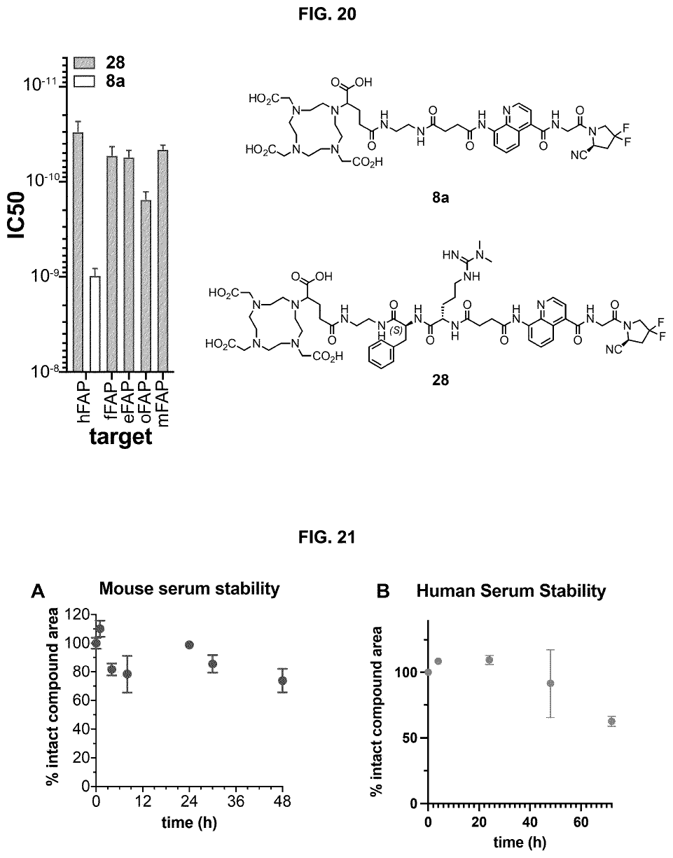

FIG. 20: In vitro validation of affinity matured FAP ligands. The histogram shows a comparison between compound 8a (comparative) and 28 performed by inhibition assay with human FAP, and the cross-reactivity of compound 28 against feline FAP (fFAP), equine FAP (eFAP), ovine FAP (oFAP) and murine FAP (mFAP). Bars represent average of IC50 values.

### Biodistribution Experiments

### Cell Culture

Upon thawing, HT-1080.hFAP and HT-1080.wt cells were kept in culture in DMEM medium supplemented with fetal bovine serum (10%, FBS) and Antibiotic-Antimycotic (1%, AA) at 37° C. and 5% CO₂. For passaging, cells were detached using Trypsin-EDTA 0.05% when reaching 80% confluence and re-seeded at a dilution of 1:4.

### 3.1 Biodistribution of C9 (177Lu]—Lu-ESV6-11-DOTAGA)

### Tumor Implantation

Tumor cells were grown to 80% confluence in Dulbecco's Modified Eagle Medium (DMEM) media with 10% fetal bovine serum (FBS) and 1% antibiotic-antimycotic and detached with Trypsin-EDTA 0.05%. HT-1080.hFAP, cells (FAP positive cells) and HT-1080.wt, cells (FAP negative cells) were separately resuspended in Hanks' Balanced Salt Solution medium. Aliquots of 5×10⁶ cells of both lines (100 μL of suspension) were injected subcutaneously in the right and left flanks respectively of female athymic Balb/c AnNRj-Foxn1 mice (6 to 8 wk of age, Janvier).

### Radiolabeling Protocol of Compound 28 (ESV6-11-DOTAGA)

75 μL of ESV6-11-DOTAGA (compound 28 1 mM solution in mQ Water, 4% DMSO) were diluted with 142 μL of 1M sodium acetate buffer, pH=8. Then, 8 μL of [¹⁷⁷Lu](13.5 MBq) were added and the solution was heated at 95° C. for 10 minutes at 700 rpm. The solution was cooled down and then diluted with 1275 μL of PBS and checked via Radio-HPLC (injection of 100 μL), showing a single peak with >95% of conversion. Mice were randomized (n=3, 4 or 5 per group) and injected intravenously with preparations of [¹⁷⁷Lu]—Lu-ESV6-11-DOTAGA (100 μL=0.9 MBq=5 nmol/mouse) (45.1 MBq/Kg; 250 nmol/Kg).

### In Vivo Biodistribution Experiment

C9 ([177Lu]—Lu-ESV6-11-DOTAGA) was injected at the dose of 250 nmol/Kg and animals were sacrificed 1 h, 4 h, 17 h, 24 h, 48 h and 72 h after intravenous administration.

### Brief Description of the Results

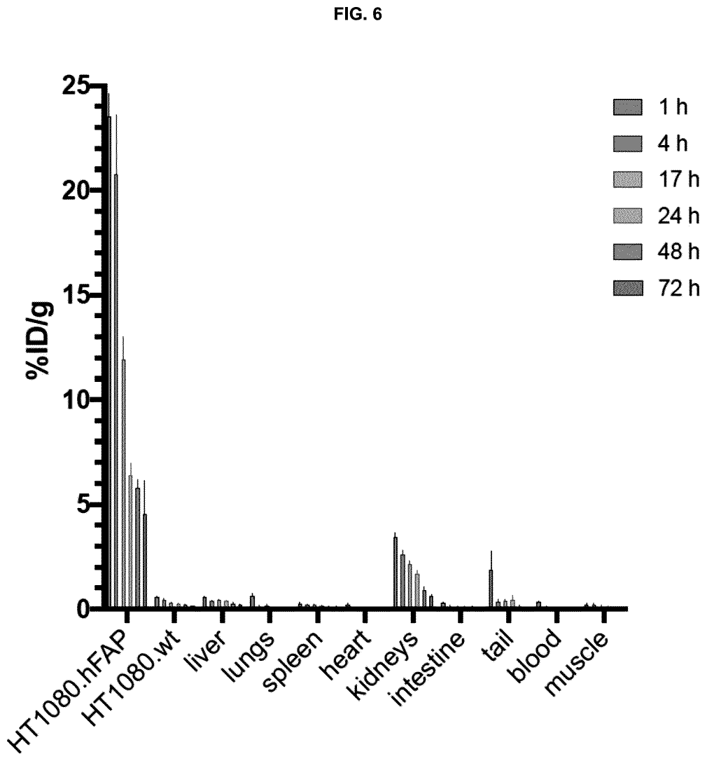

C9 ([⁷⁷Lu]—Lu-ESV6-11-DOTAGA) accumulated selectively in FAP-expressing HT-1080 tumors already 1 h after intravenous injection (23.6% ID/g), with a sustained uptake in the lesions over the first 72 hours. The compound did not accumulate in FAP-negative tumors subcutaneously implanted in the same animal. No substantial uptake was observed in healthy organs (e.g., 7-to-1 in kidney after 1 h and 4 h) (FIG. 6).

### 3.2 Biodistribution of Compound 42 (¹⁷⁷-Lu—Bi-ESV6-11-DOTAGA)

### Tumor Implantation

Tumor cells were grown to 80% confluence in Dulbecco's Modified Eagle Medium (DMEM) media with 10% fetal bovine serum (FBS) and 1% antibiotic-antimycotic and detached with Trypsin-EDTA 0.05%. HT-1080.hFAP, cells (FAP positive cells) were resuspended in Hanks' Balanced Salt Solution medium. Aliquots of 5×10⁶ cells of both lines (100 μL of suspension) were injected subcutaneously in the right flank of female athymic Balb/c AnNRj-Foxn1 mice (6 to 8 wk of age, Janvier).

### Radiolabeling Protocol of Compound 38 (Bi-ESV6-11-DOTAGA)

82 μL of Bi-ESV6-11-DOTAGA (compound 38 1 mM solution in mQ Water, 4% DMSO) were diluted with 132 μL of 1M sodium acetate buffer, pH=8. Then, 31 μL of [¹⁷⁷Lu](15 MBq) were added and the solution was heated at 95° C. for 10 minutes at 700 rpm. The solution was cooled down and then diluted with 1255 μL of PBS and checked via Radio-HPLC (injection of 100 μL), showing a single peak with >95% of conversion. Mice were randomized (n=3, 4 or 5 per group) and injected intravenously with preparations of [¹⁷⁷Lu]—Lu—Bi-ESV6-11-DOTAGA (100 μL=1 MBq=5.5 nmol/mouse).

### In Vivo Biodistribution Experiment

Compound 42 ([177Lu]—Lu—Bi-ESV6-11-DOTAGA) was injected at the dose of 275 nmol/Kg and animals were sacrificed 4 h, 24 h, 48 h and 72 h after intravenous administration.

### Brief Description of the Results

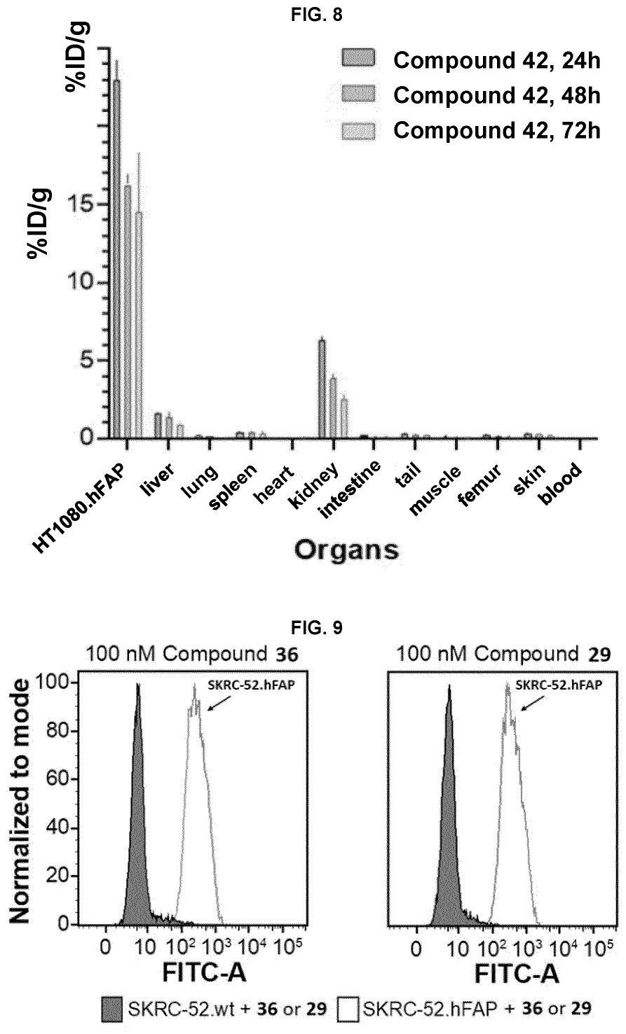

Compound 42 ([¹⁷⁷Lu]—Lu—Bi-ESV6-11-DOTAGA) accumulated selectively in FAP-expressing HT-1080 tumors with a sustained uptake in the lesions over the first 72 hours (˜14% ID/g) (FIG. 8).

### 3. FACS Experiment

### FACS Experiment

Cells were detached from culture plates using Accutase cell detachment solution, counted and suspended to a final concentration of 1.25×10≢cell/100 μL in a solution of FACS buffer (1% bovine serum albumin, 2 mM EDTA, in 500 mL PBS pH 7.4). Aliquots of 2.5×10≡cells (200 μL) were spun down and resuspended in solutions of compound 36 and compound 29 (100 μL; 100 nM) in FACS buffer and incubated on ice in the dark for 1 h. Cells were washed once with 200 μl FACS (1% v/v)/PBS (pH=7.4), spun down, resuspended in a solution of FACS buffer and analyzed via a BD LSRFortessa™ Cell Analyzer (Biosciences). FlowJo Version 8.7 (Treestar) was used for the data analysis and visualization.

### Brief Description of the Results

Compounds 36 and 29 were tested in-vitro against hFAP-positive cells in flow cytometry experiments (FIG. 9). Both compounds stained tumor cells presenting FAP antigens (SKRC-52.hFAP), while no binding on wild-type cell lines (FAP-negative SKRC-52.wt) was detected.

### 4. Confocal Experiment

### Confocal Experiment

SK—RC-52.hFAP and SK—RC-52.wt cells were seeded into g-Slide 8 Well high Glass Bottom at a density of 5×10⁴ cells per well in 300 μL of RPMI medium supplemented with 10% fetal calf serum (FCS), AA, and Hepes (10 mM) and allowed to grow overnight under standard culture conditions. Hoechst 33342 nuclear dye was used to stain nuclear structures. The culture medium was replaced with fresh medium containing either compound 36 or 29 (150 nM). Randomly selected colonies were imaged 1 h after incubation on the confocal microscope Zeiss LSM 880 airyscan (Zeiss). Fiji was used for the data processing and visualization.

### Brief Description of the Results

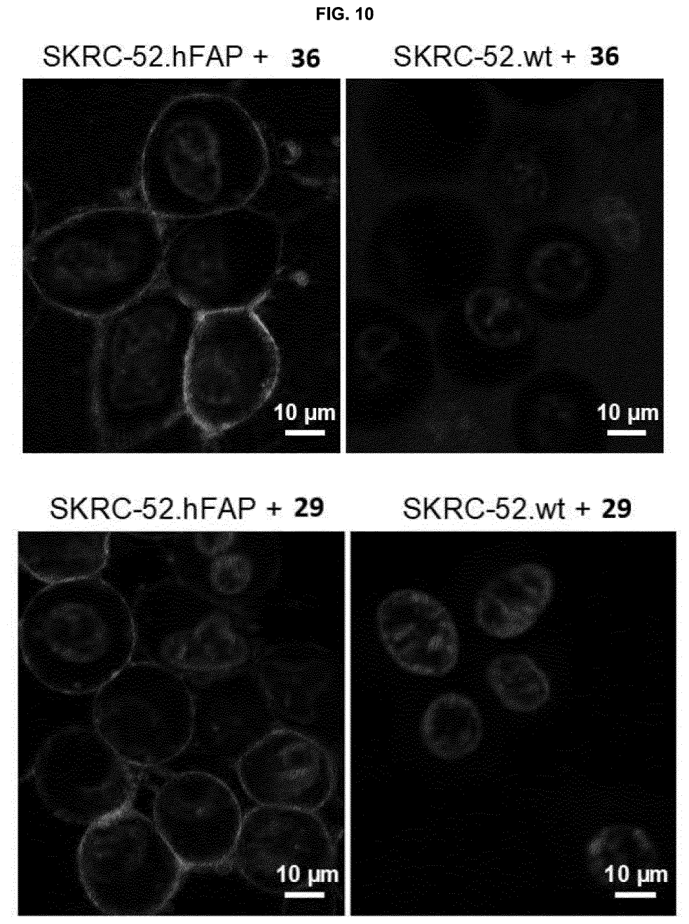

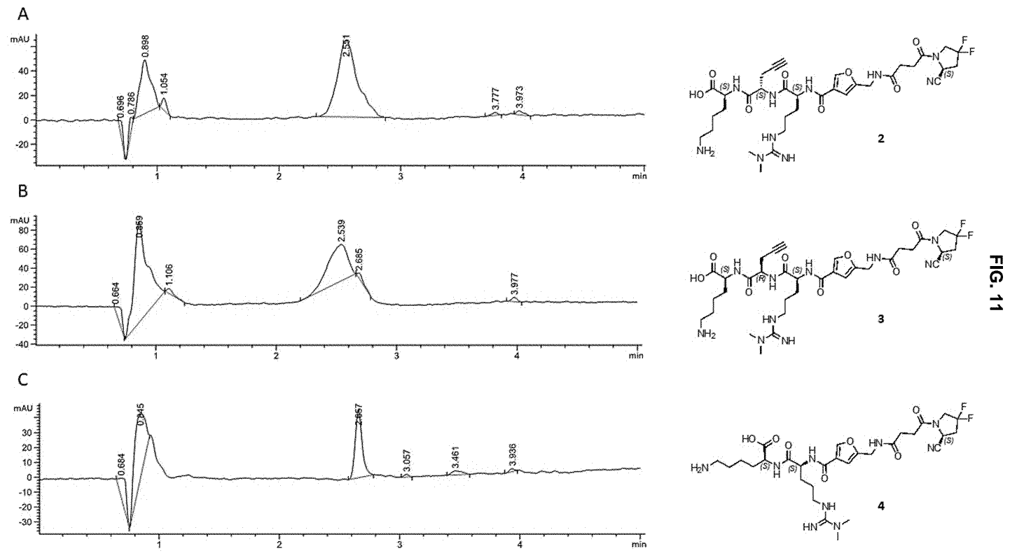

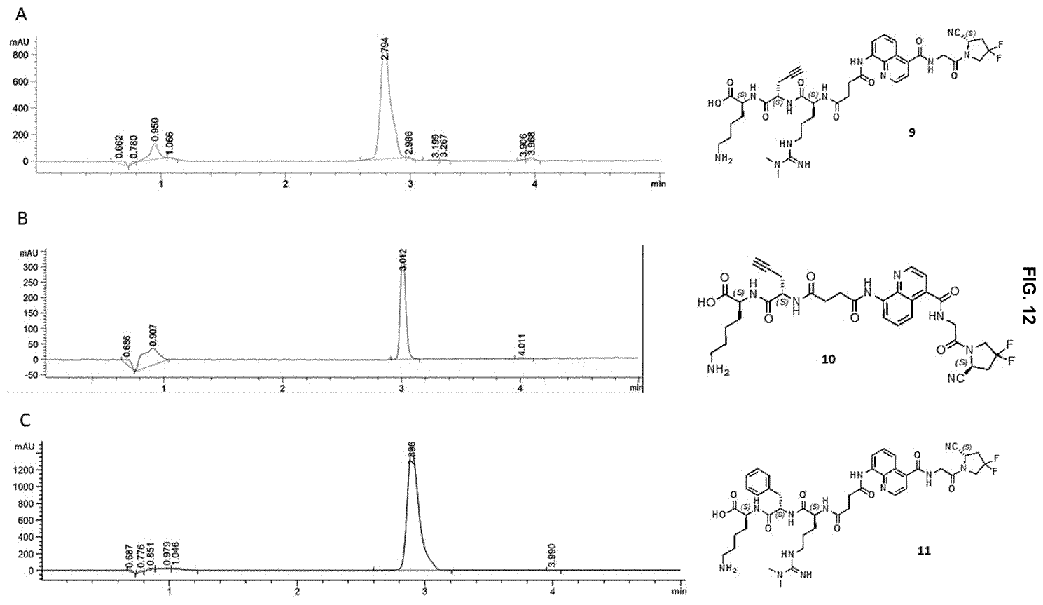

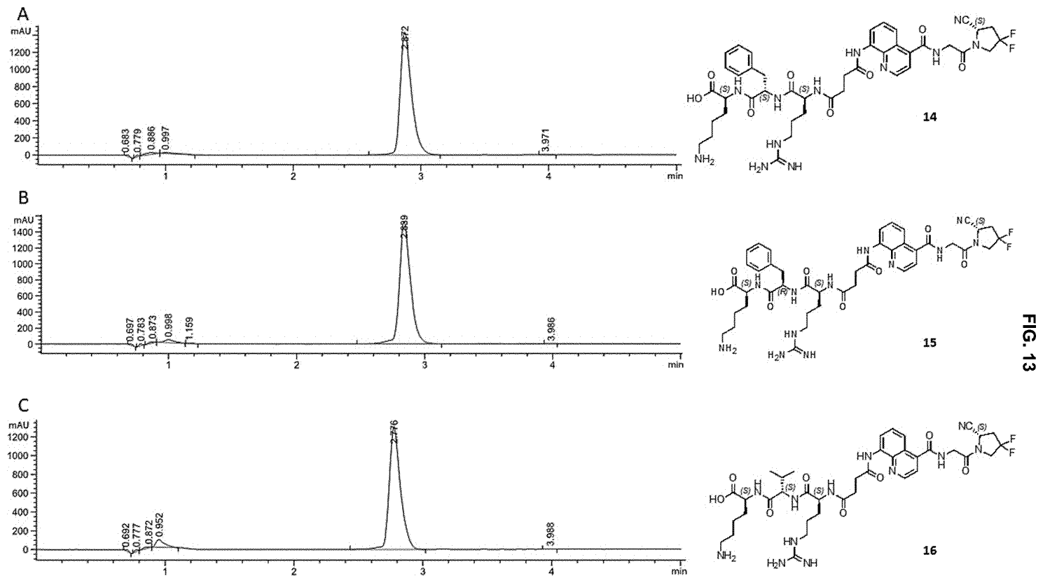

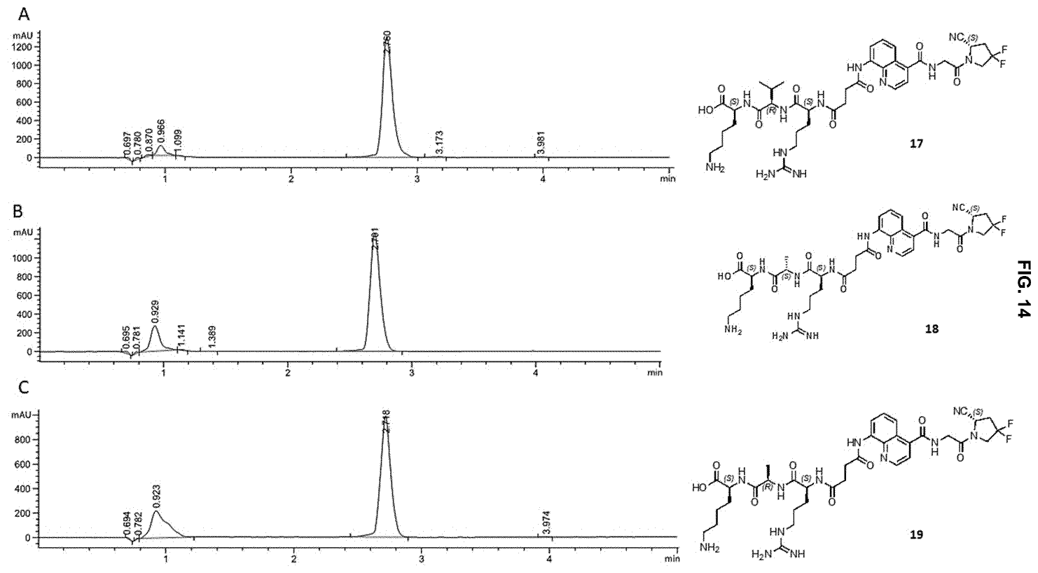

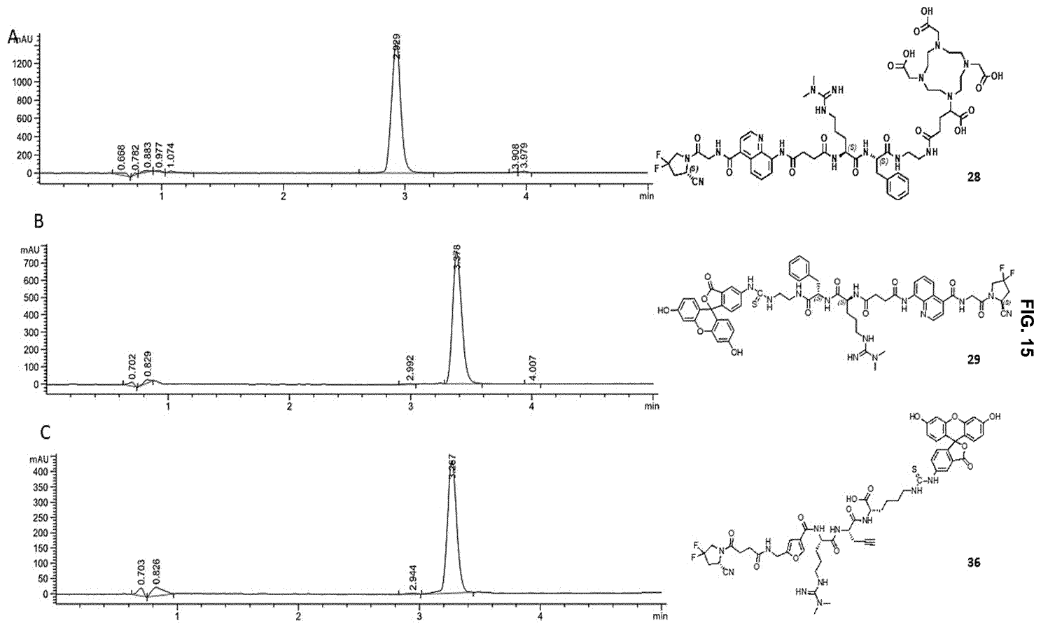

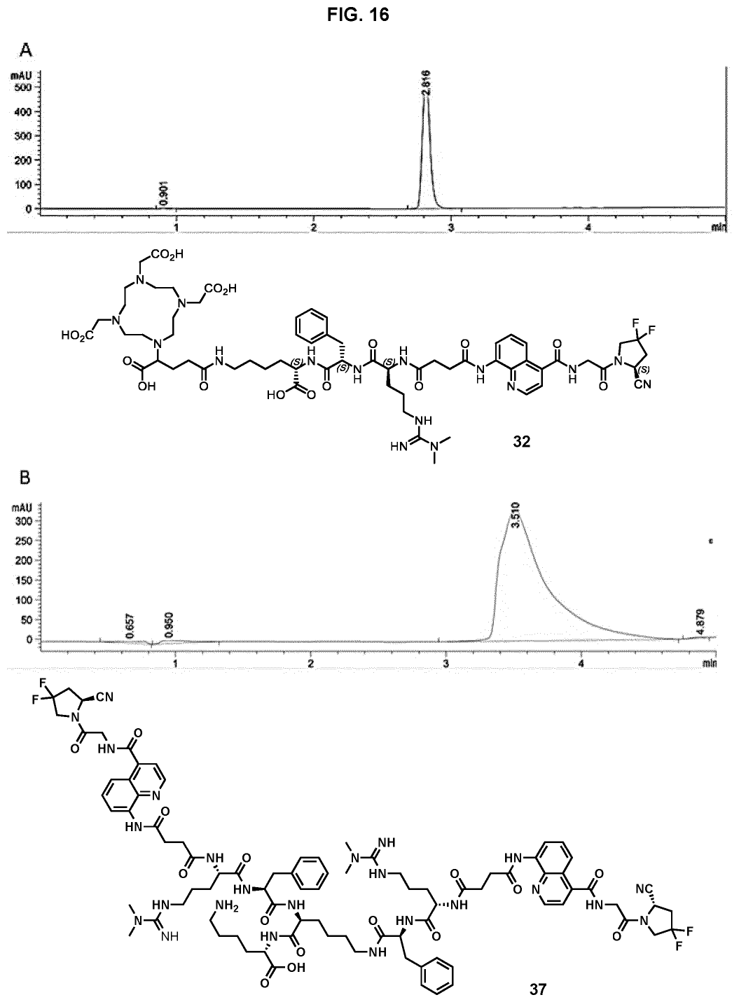

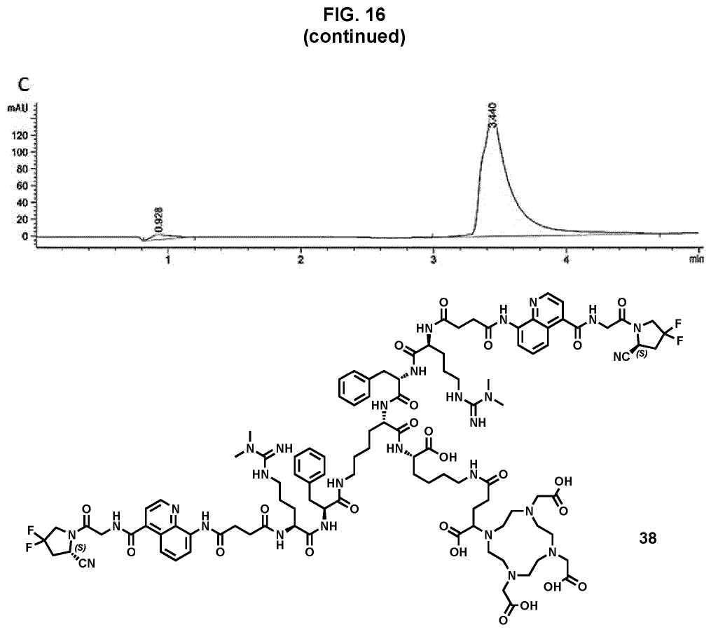

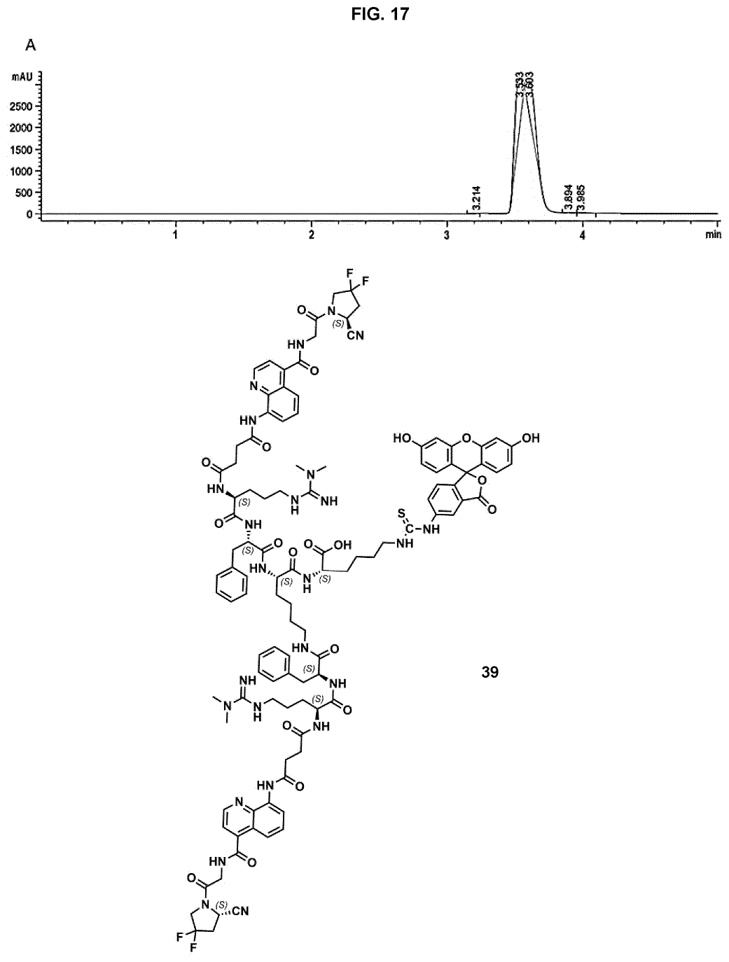

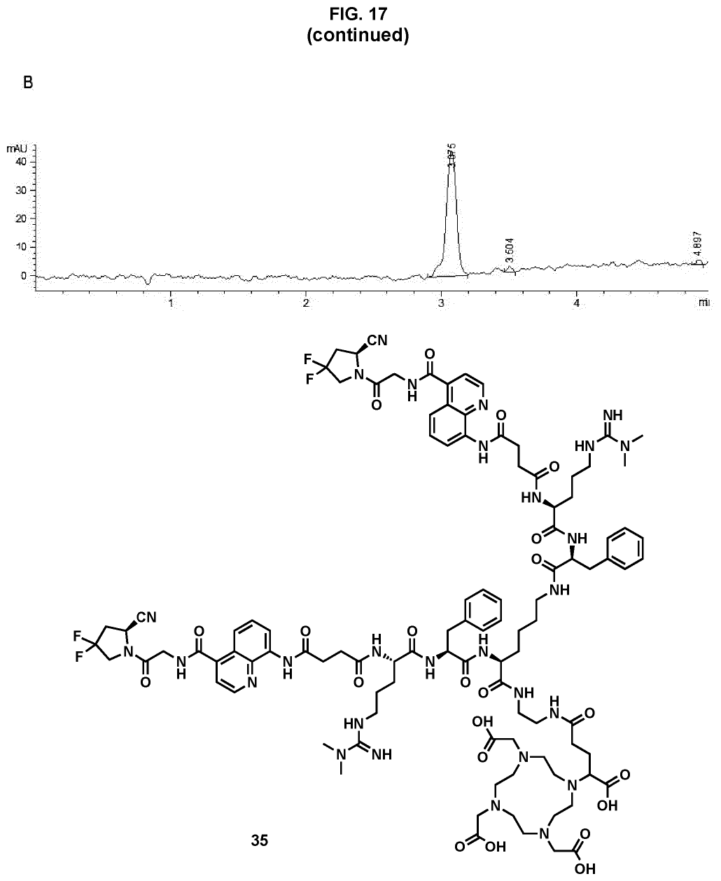

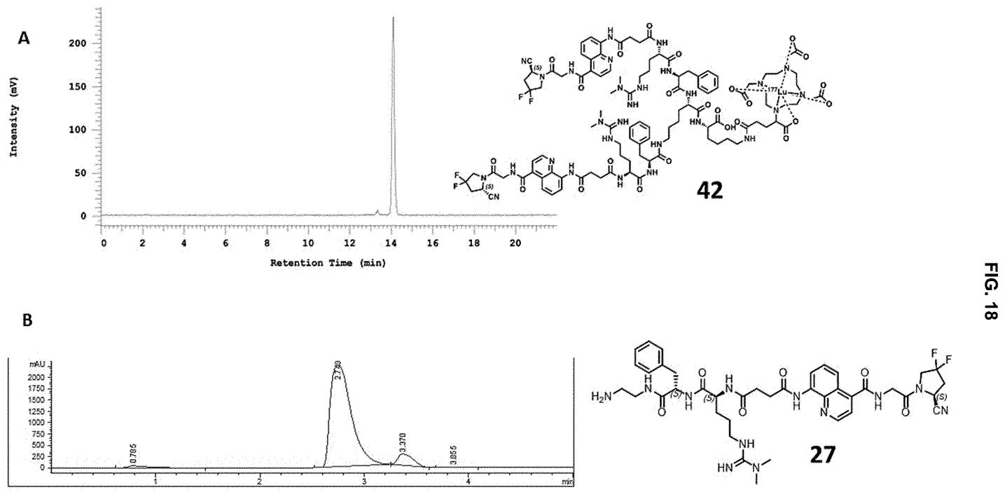

Membrane-specific staining of compounds 36 and 29 was observed in confocal microscopy experiments (FIG. 10) performed on living cells. Internalization was not observed both for compound 36 and for compound 29.

### 5. Serum Stability

In two separate Eppendorf, 4 μL of compound 28 and 38 (1 mM solution in DMSO) and 36 μL of serum for each time point were mixed. After each specific time point, 40 μL of mixture were freezed in liquid nitrogen and then stored at −20° C. Once all the samples were collected, samples were defrosted at 24° C. and 300 μL of ACN were added. The samples were then centrifuged at 10′000 RPM for 5 minutes. 200 μL of the supernatant were transferred to a second Eppendorf tube and dried under vacuum for 2 h. The residue was carefully resuspended in 30 μL of mQ Water:acetonitrile=9:1 mixture (with 0.1% v/v of formic acid) and analysed by LC-MS. Each time point was performed in triplicate. The percentage of intact compound 20 and 22 was determined extracting the area of the UV peak. The obtained values were normalized on the average area at time point corresponding to 0 hours.

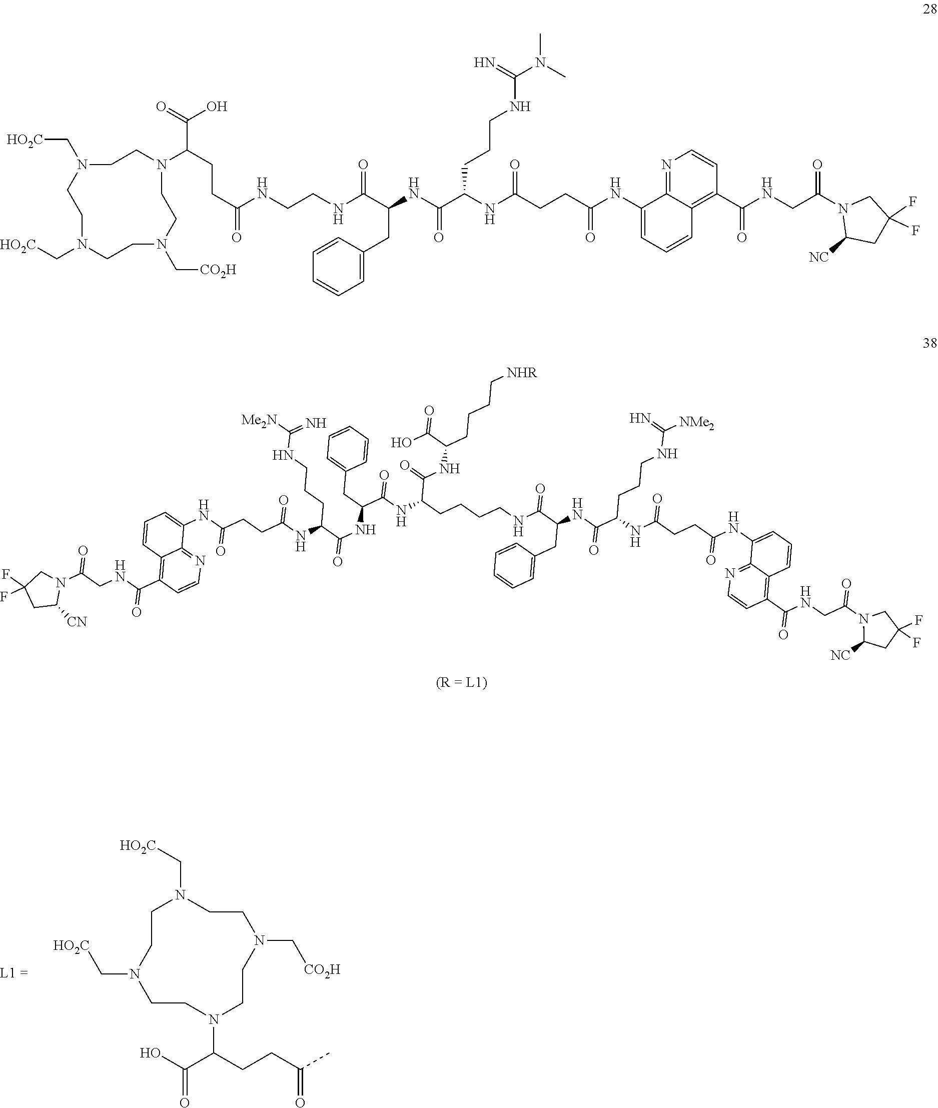

### Brief Description of the Results

FIG. 21A: Mouse serum stability of 28 reported at time 0, 1 h, 4 h, 8 h, 24 h, 30 h and 48 h. B: Human serum stability of compound 38 reported at time 0, 4 h, 24 h, 48 h and 72 h. Compound 28 showed particularly favourable in vitro stability in mouse serum, with a half-life of more than 48 hours.

### 6. Conjugates

Particularly preferred compounds disclosed herein are the conjugates listed in Table 4 below.

TABLE 4
Conjugates

| Com- | | | |
|---|---|---|---|
| pound | | | |
| # | Structure | | |
| C1 | 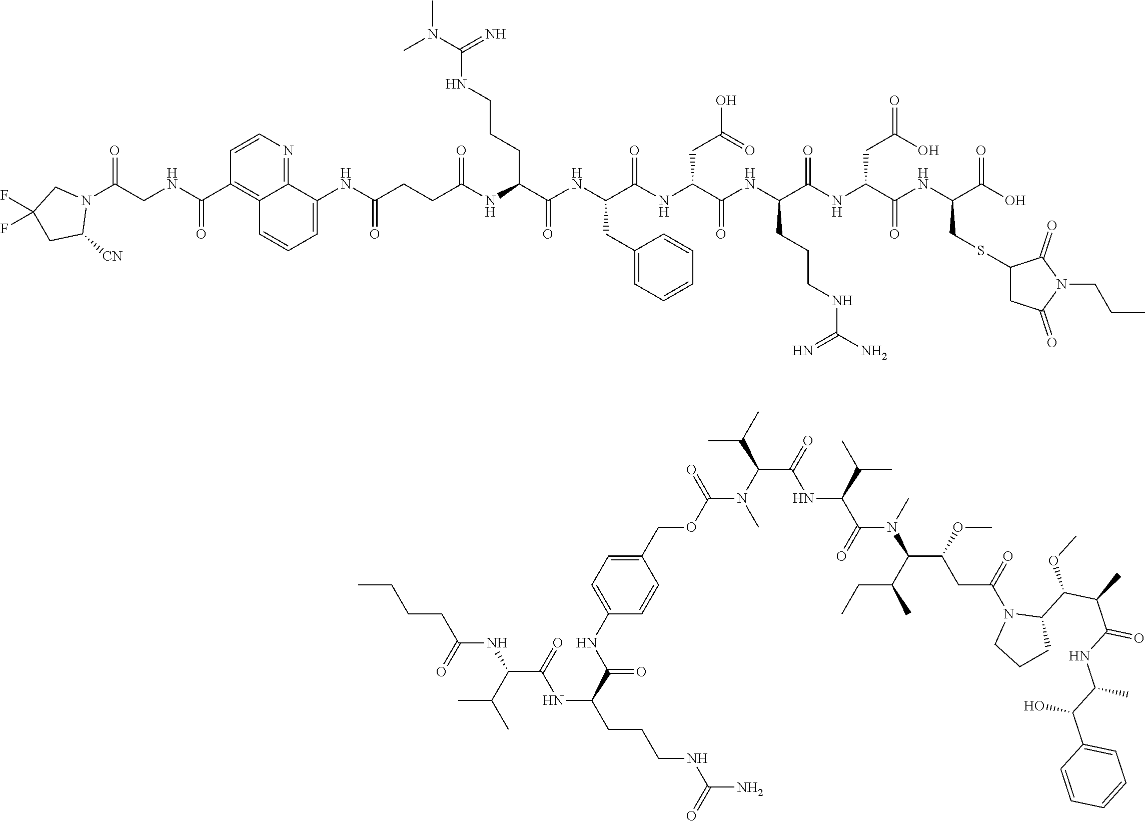 | | |
| C2 | 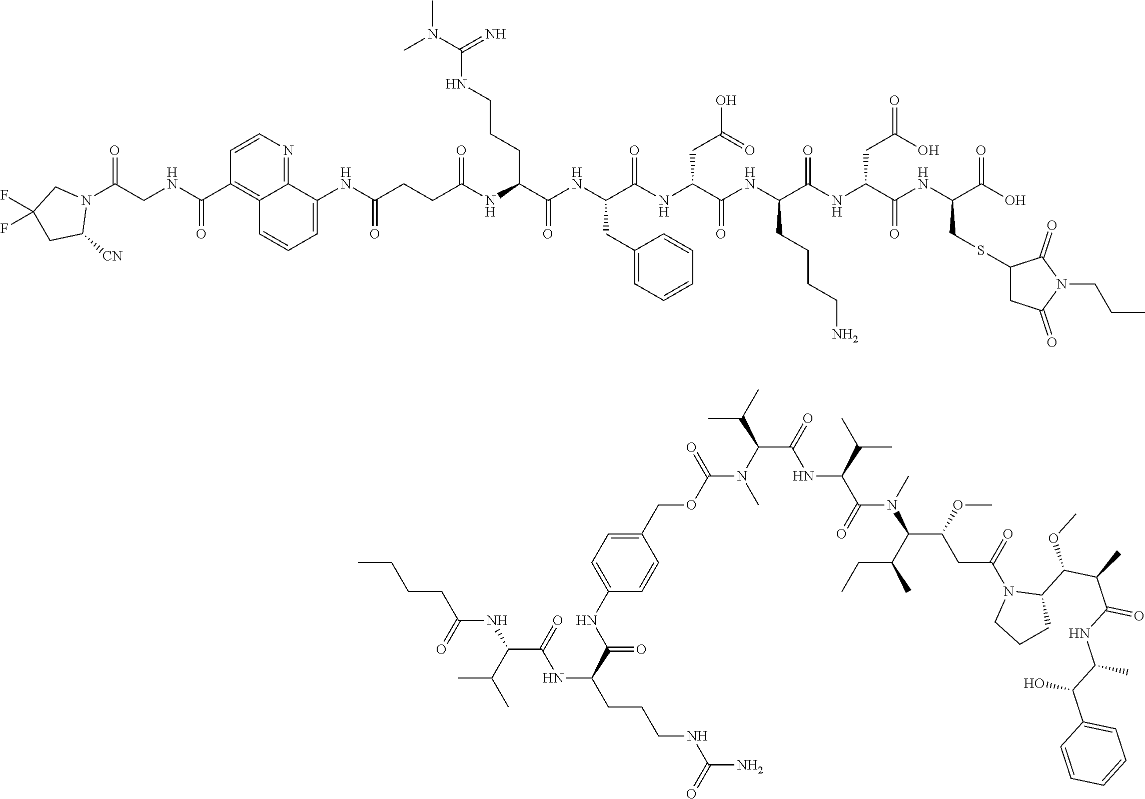 | | |
| C3 | 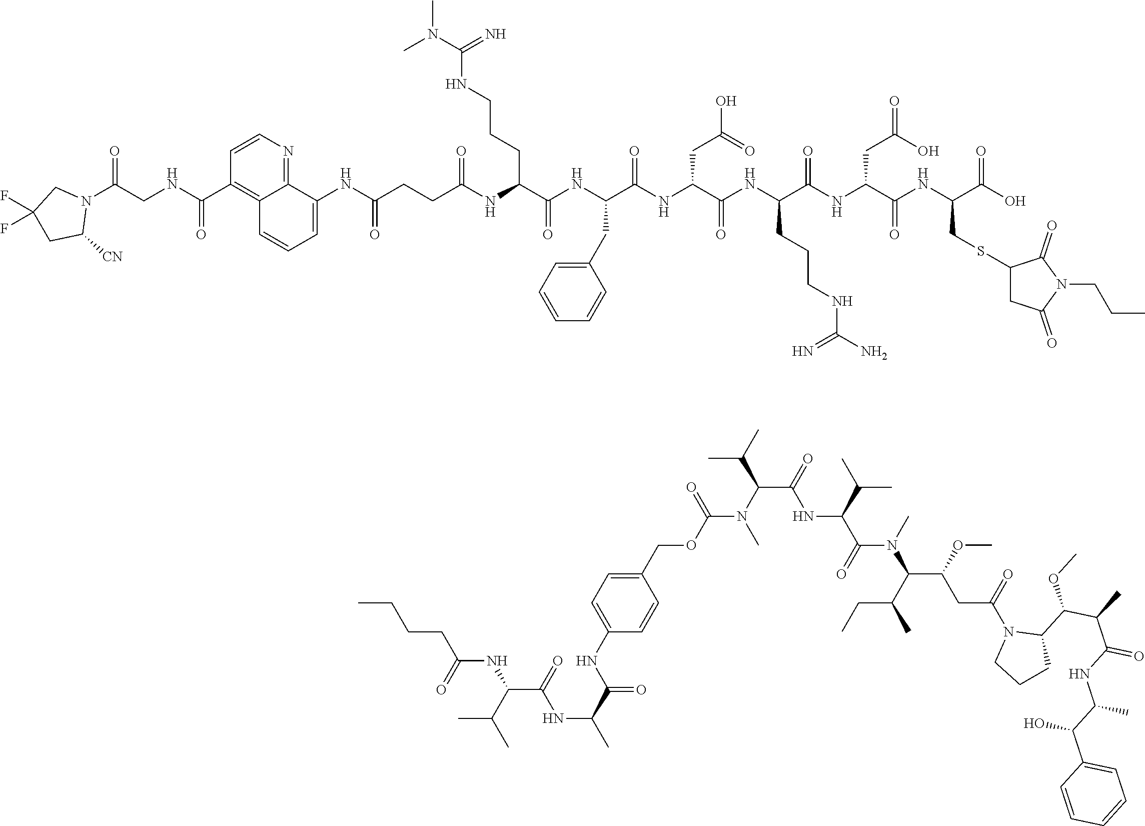 | | |
| C4 | 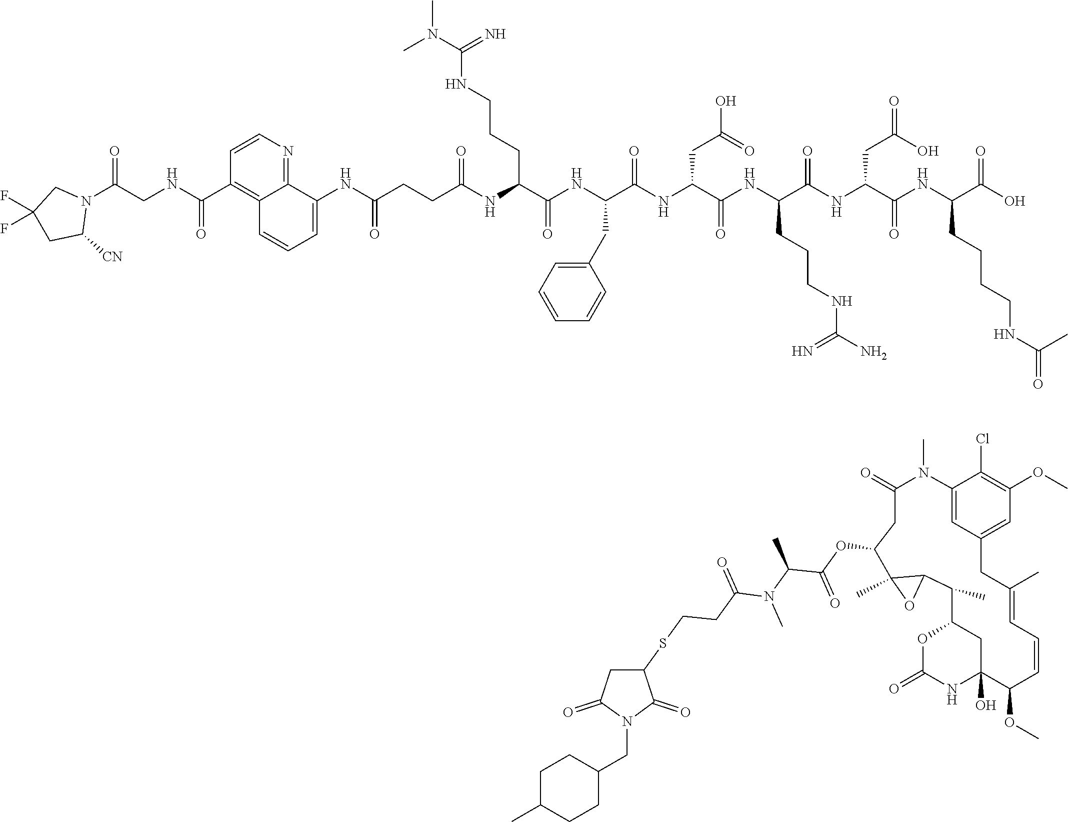 | | |
| C5 | 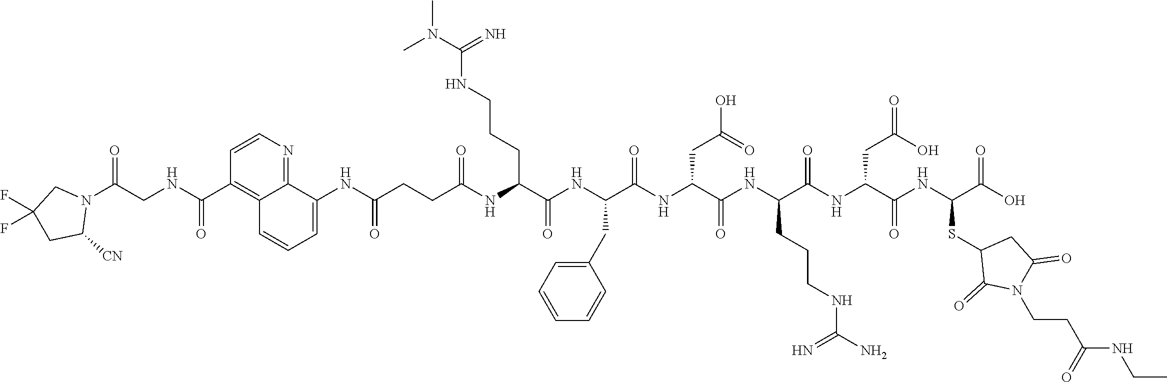 [SMILES: CCNC(=O)CCN1C(=O)CC(S[C@@H](NC(=O)[C@@H](CC(=O)O)NC(=O)[C@@H](CCCNC(=N)N)NC(=O)[C@@H](CC(=O)O)NC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)C(=O)O)C1=O] 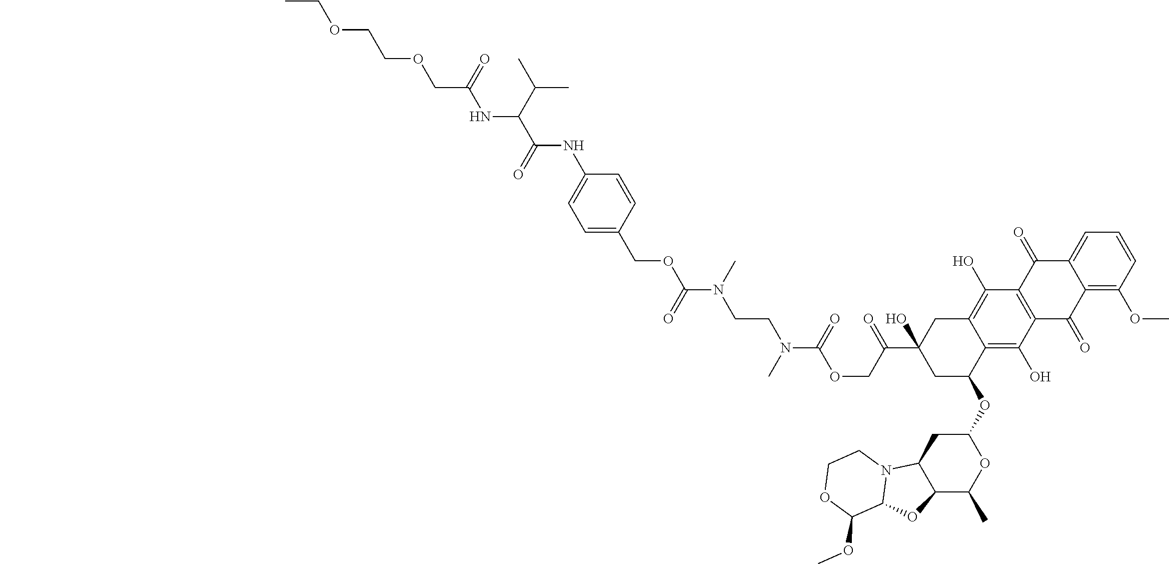 | | |
| C6 | 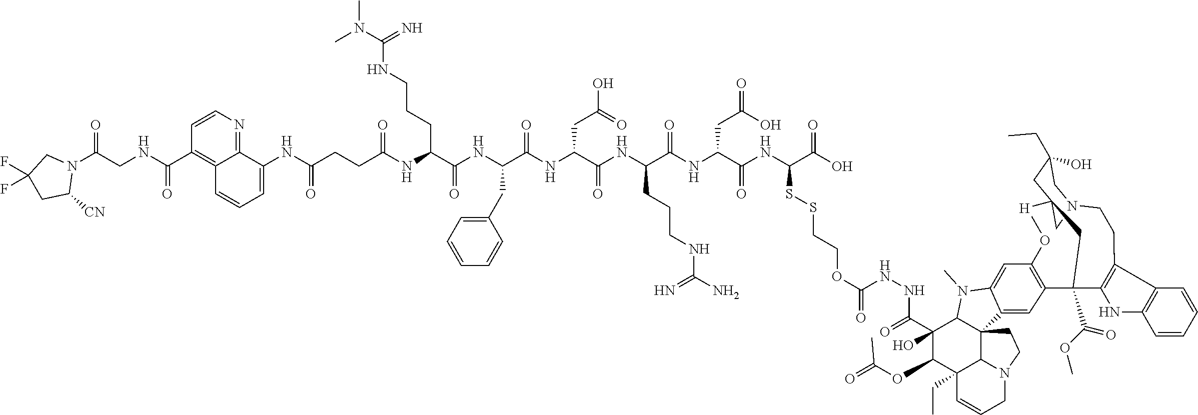 | | |
| C7 | 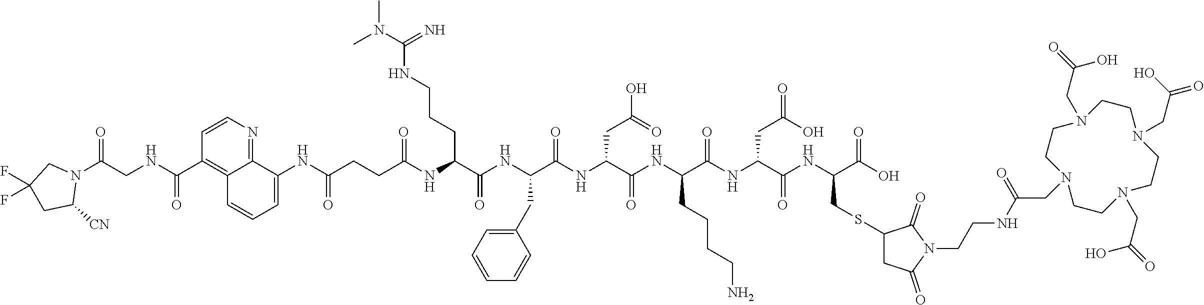 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)N[C@H](CC(=O)O)C(=O)N[C@H](CCCCN)C(=O)N[C@H](CC(=O)O)C(=O)N[C@H](CSC1CC(=O)N(CCNC(=O)CN2CCN(CC(=O)O)CCN(CC(=O)O)CCN(CC(=O)O)CC2)C1=O)C(=O)O] | | |
| C8 | 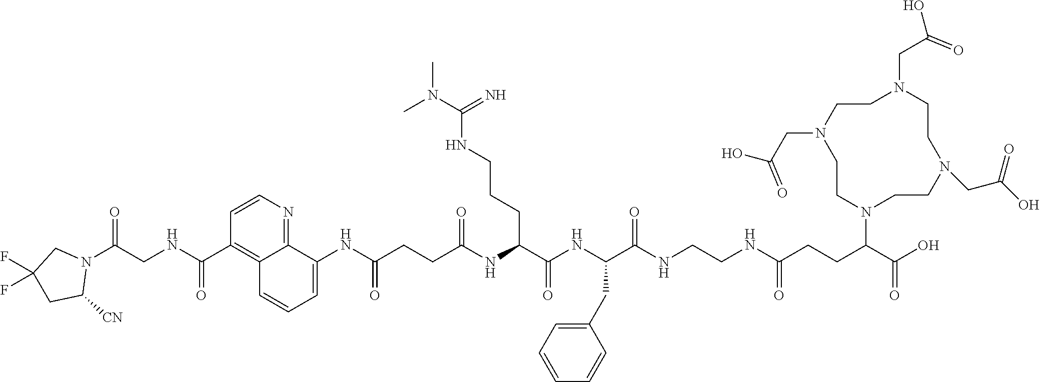 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCNC(=O)CCC(C(=O)O)N1CCN(CC(=O)O)CCN(CC(=O)O)CCN(CC(=O)O)CC1] | | |
| C9 | 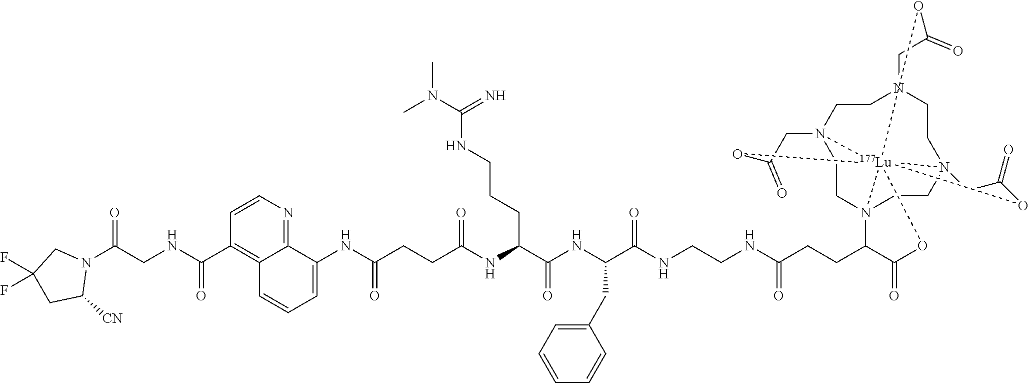 | | |
| C10 | 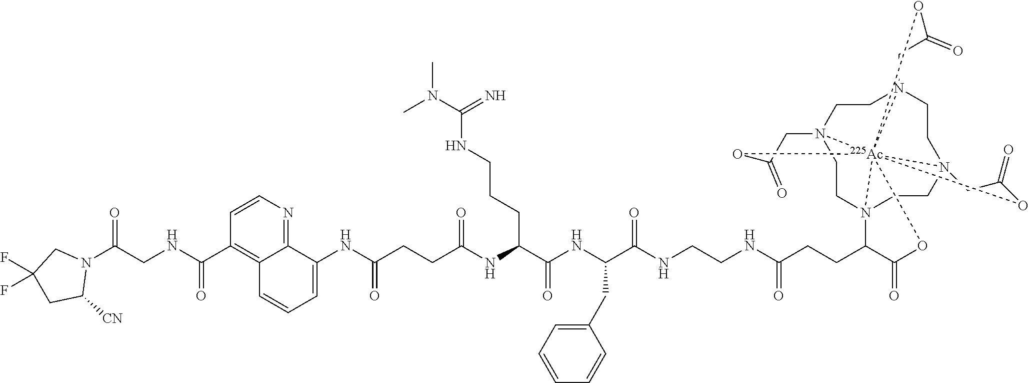 | | |
| C11 | 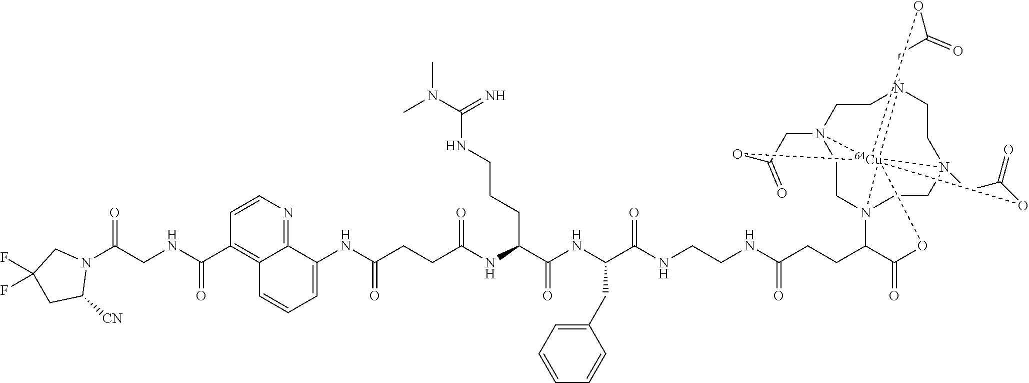 | | |
| C12 | 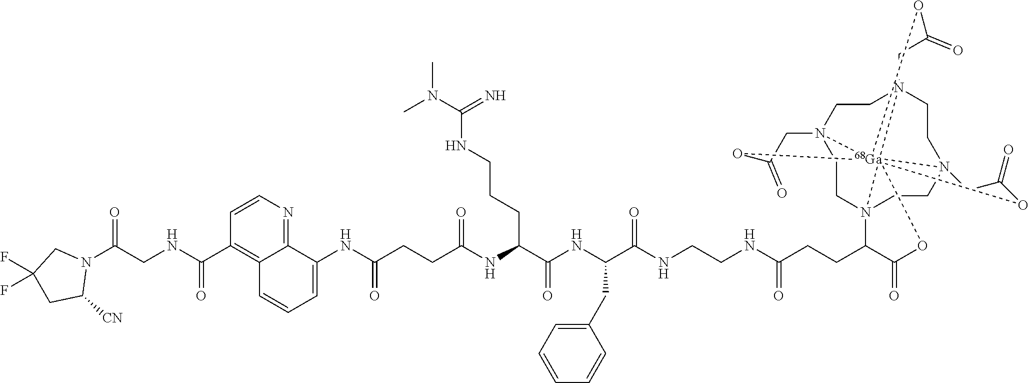 | | |
| C13 | 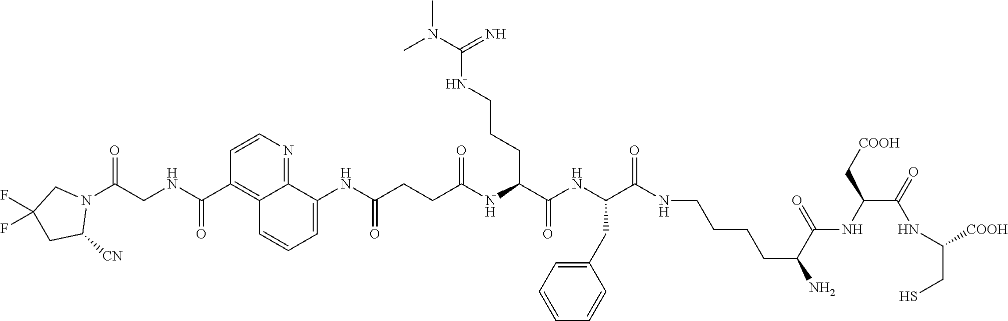 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](N)C(=O)N[C@@H](CC(=O)O)C(=O)N[C@@H](CS)C(=O)O] | | |
| C14 | 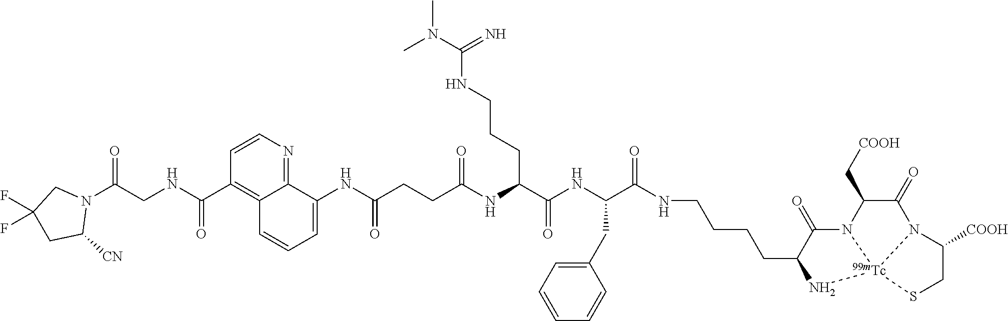 | | |
| C15 | 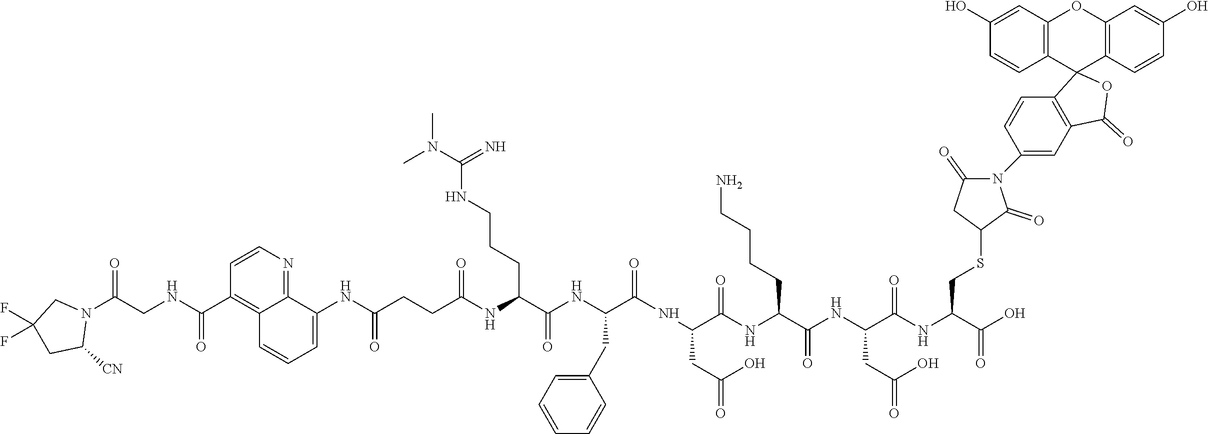 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)N[C@@H](CC(=O)O)C(=O)N[C@@H](CCCCN)C(=O)N[C@@H](CC(=O)O)C(=O)N[C@@H](CSC1CC(=O)N(c2ccc3c(c2)C(=O)OC32c3ccc(O)cc3Oc3cc(O)ccc32)C1=O)C(=O)O] | | |
| C16 | 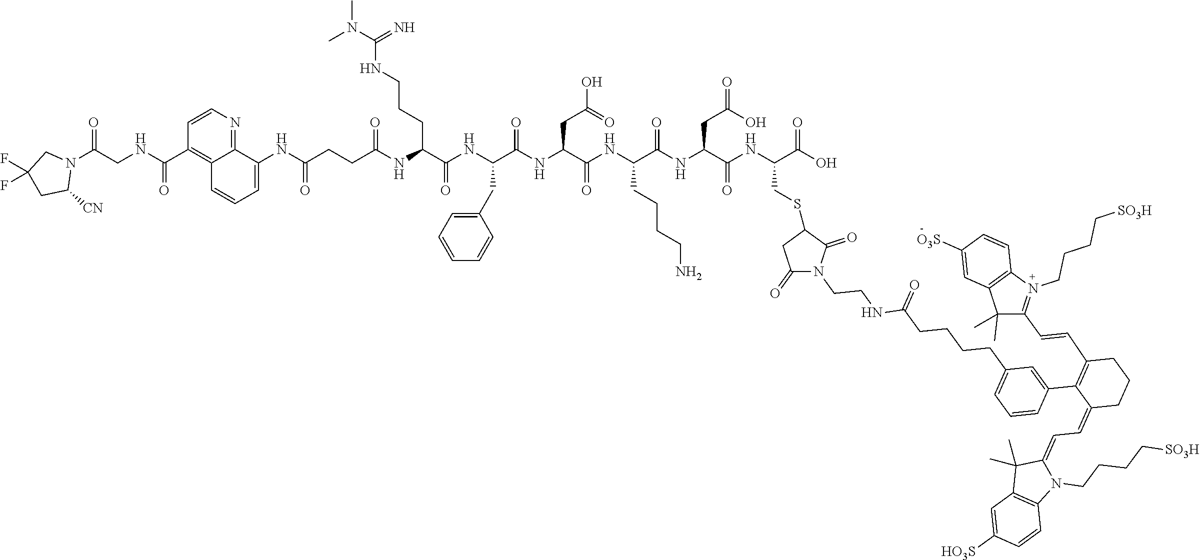 | | |
| C17 | 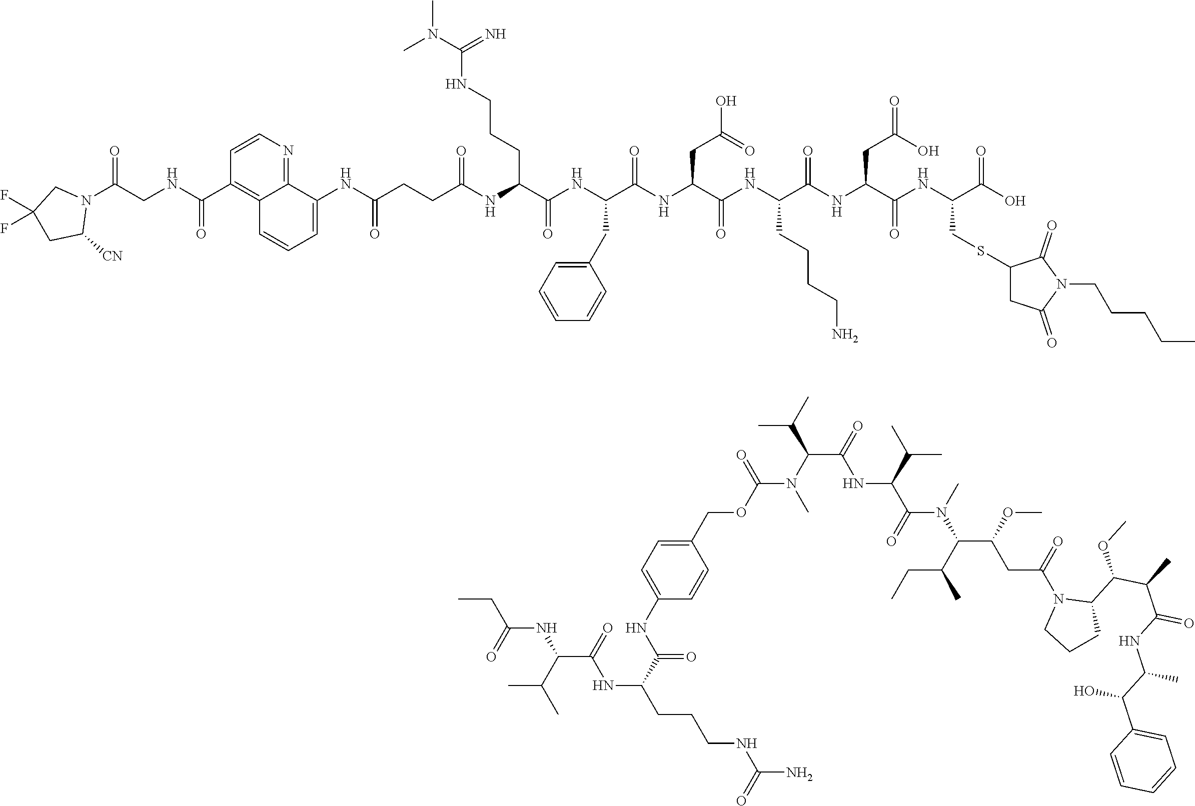 | | |
| C18 | 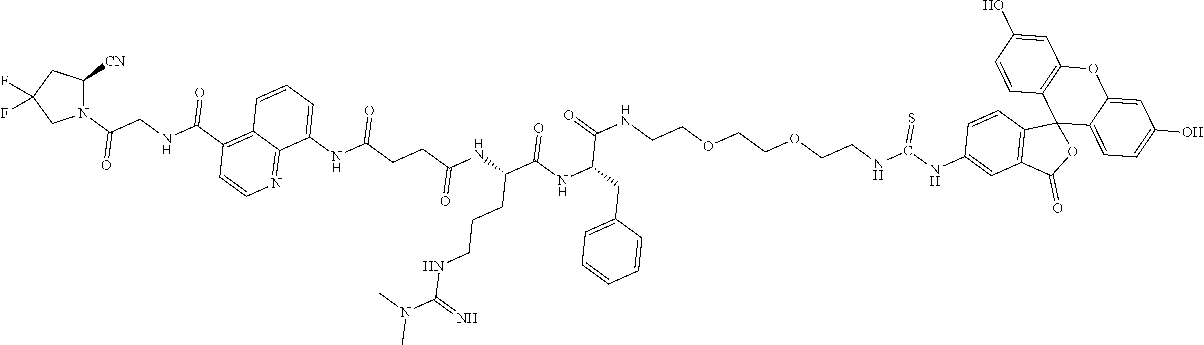 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCOCCOCCNC(=S)Nc1ccc2c(c1)C(=O)OC21c2ccc(O)cc2Oc2cc(O)ccc21] | | |
| C19 | 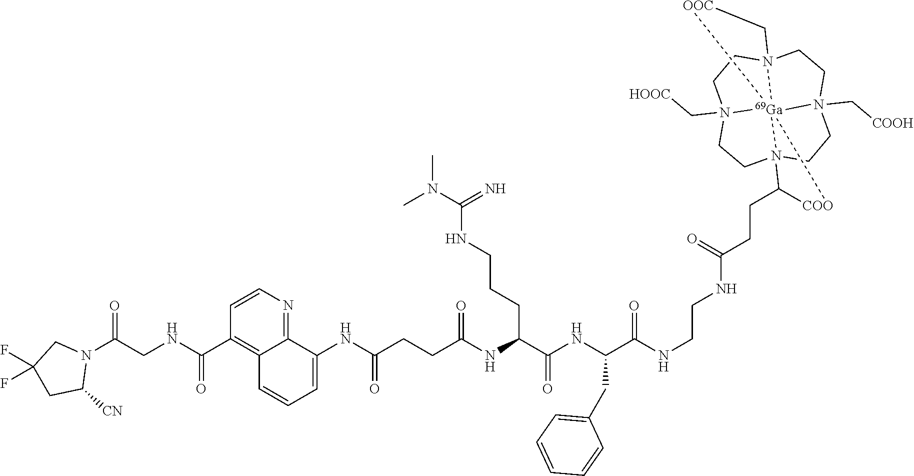 | | |
| C20 | 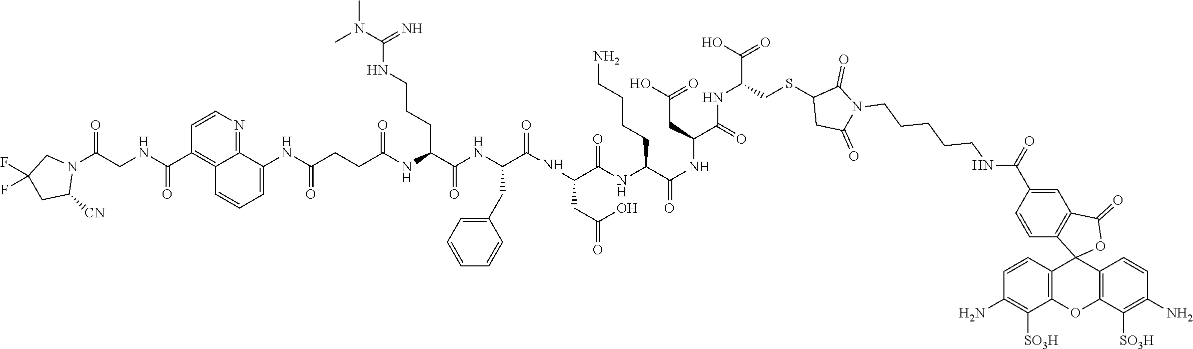 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)N[C@@H](CC(=O)O)C(=O)N[C@@H](CCCCN)C(=O)N[C@@H](CC(=O)O)C(=O)N[C@@H](CSC1CC(=O)N(CCCCCNC(=O)c2ccc3c(c2)C(=O)OC32c3ccc(N)c(S(=O)(=O)O)c3Oc3c2ccc(N)c3S(=O)(=O)O)C1=O)C(=O)O] | | |
| C21 | 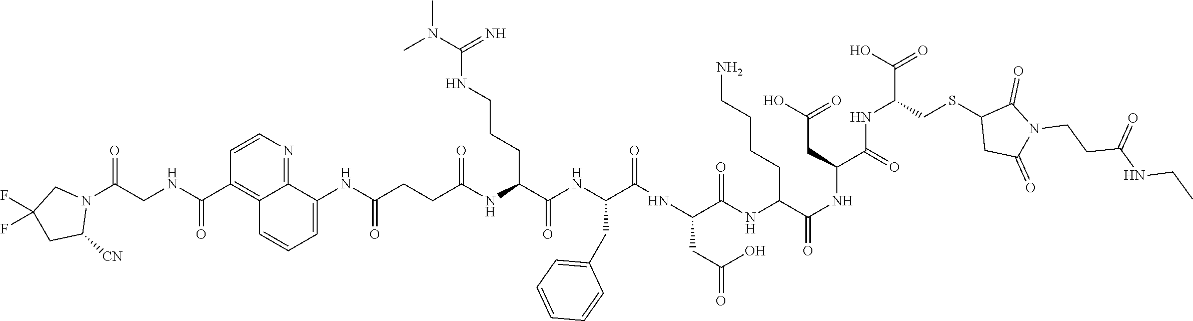 [SMILES: CCNC(=O)CCN1C(=O)CC(SC[C@H](NC(=O)[C@H](CC(=O)O)NC(=O)C(CCCCN)NC(=O)[C@H](CC(=O)O)NC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)C(=O)O)C1=O] 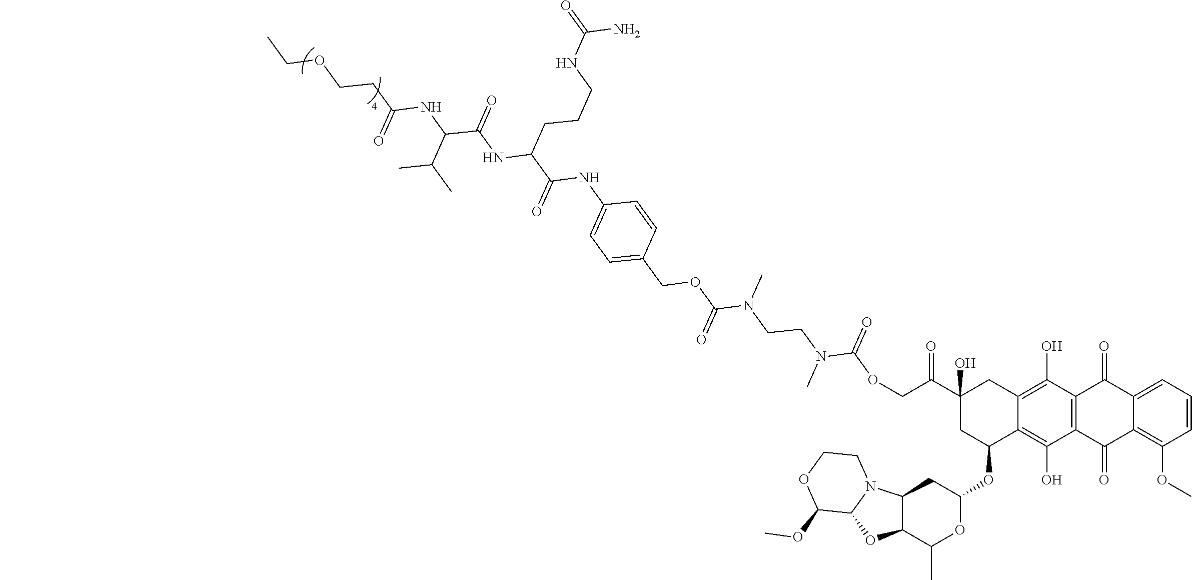 | | |
| C22 | 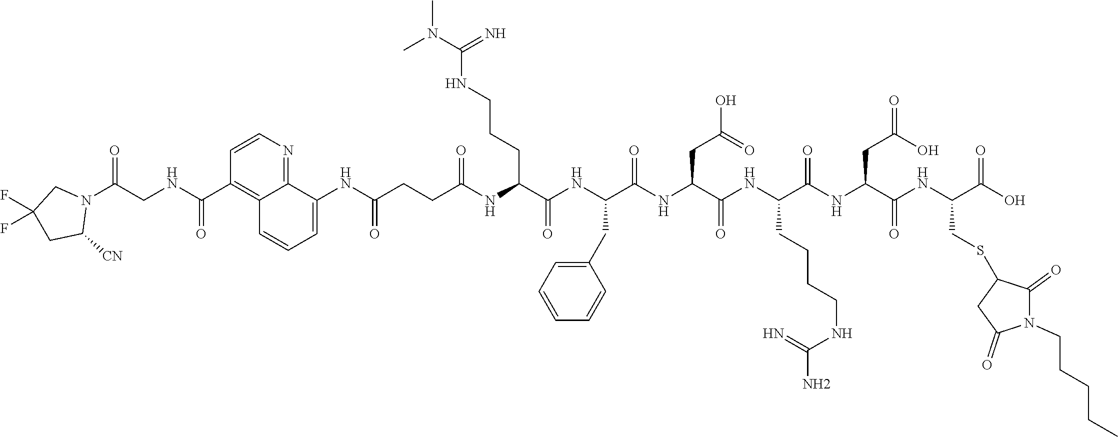 [SMILES: CCCCCN1C(=O)CC(SC[C@H](NC(=O)[C@H](CC(=O)O)NC(=O)[C@H](CCCCNC(=N)N)NC(=O)[C@H](CC(=O)O)NC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)C(=O)O)C1=O] 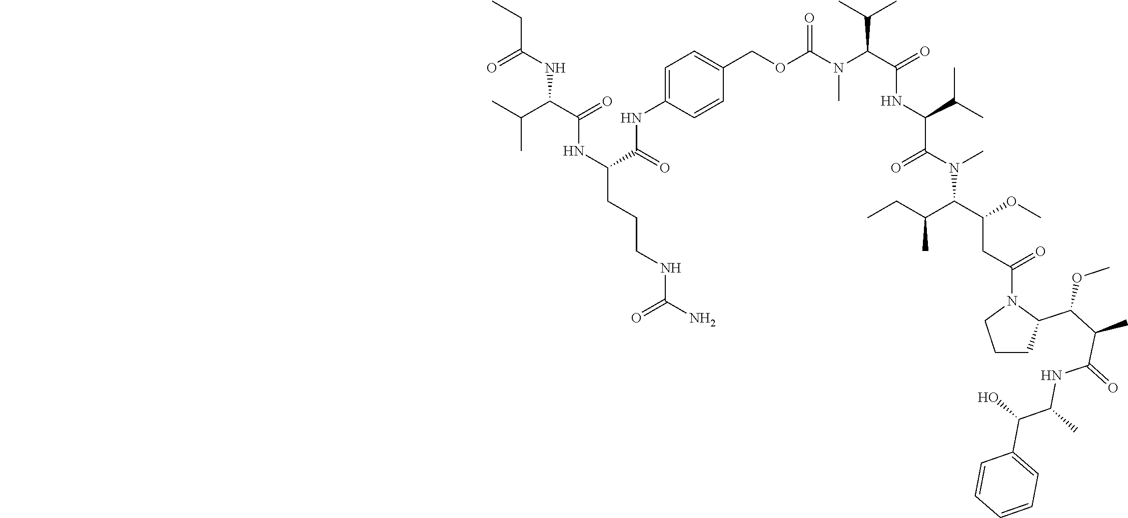 | | |
| C23 | 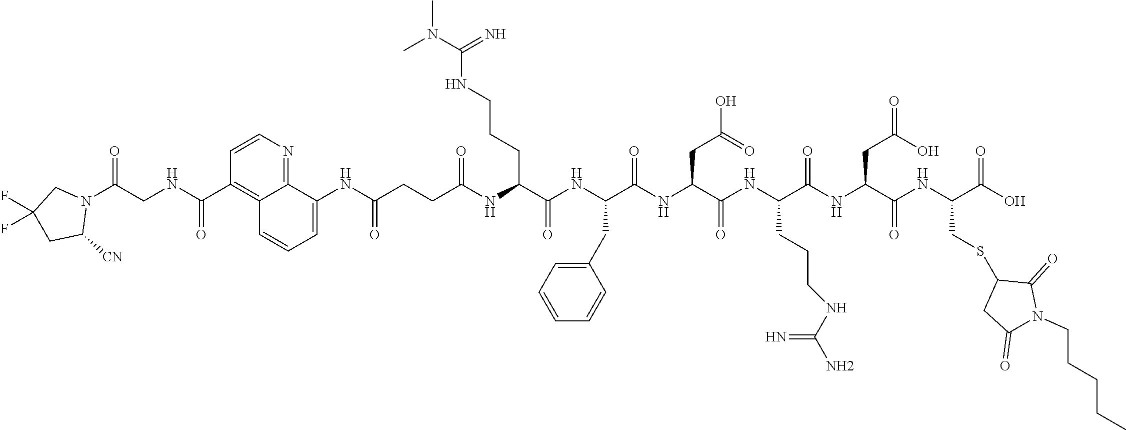 [SMILES: CCCCCN1C(=O)CC(SC[C@H](NC(=O)[C@H](CC(=O)O)NC(=O)[C@H](CCCNC(=N)N)NC(=O)[C@H](CC(=O)O)NC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)C(=O)O)C1=O] 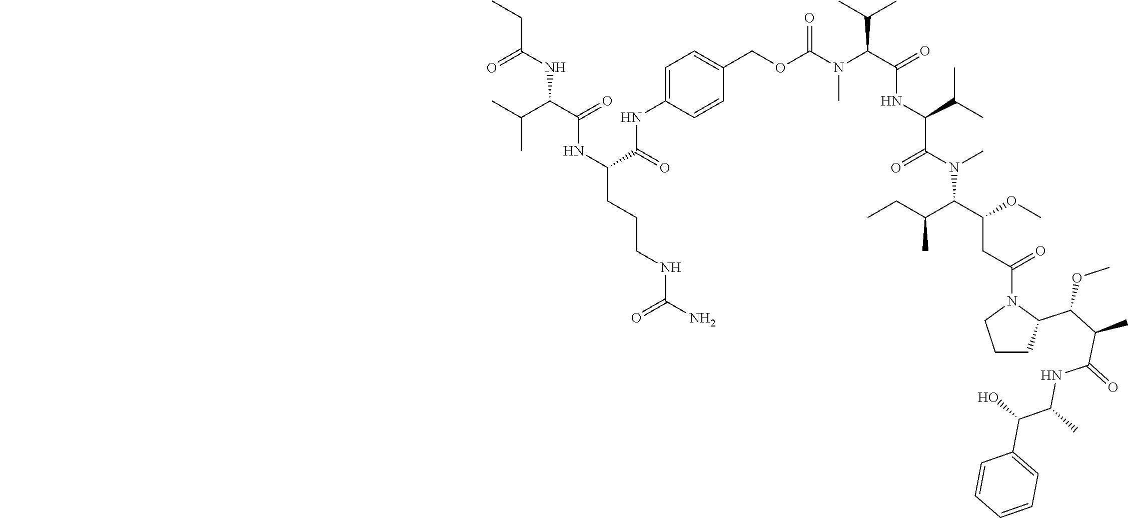 | | |
| C24 | 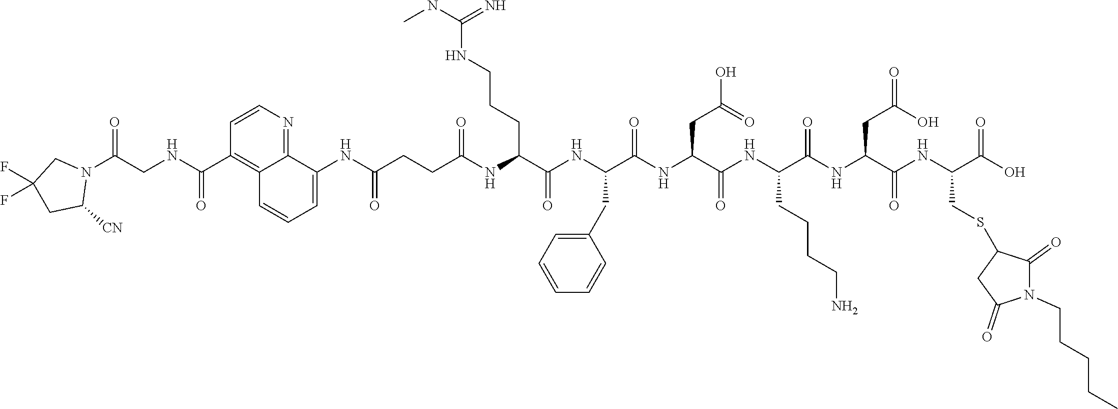 [SMILES: CCCCCN1C(=O)CC(SC[C@H](NC(=O)[C@H](CC(=O)O)NC(=O)[C@H](CCCCN)NC(=O)[C@H](CC(=O)O)NC(=O)[C@H](Cc2ccccc2)NC(=O)[C@H](CCCNC(=N)NC)NC(=O)CCC(=O)Nc2cccc3c(C(=O)NCC(=O)N4CC(F)(F)C[C@H]4C#N)ccnc23)C(=O)O)C1=O] 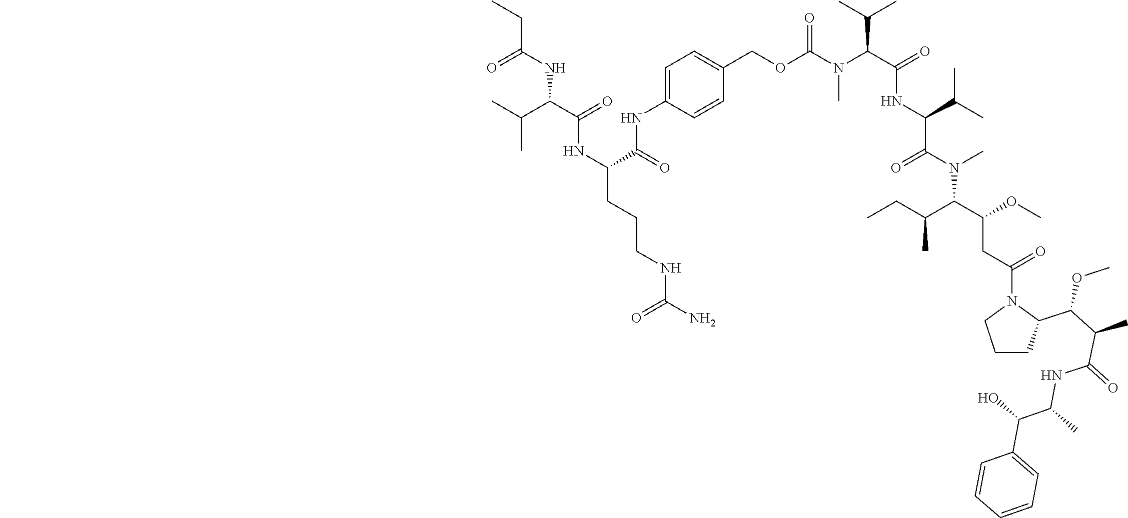 | | |
| C25 | 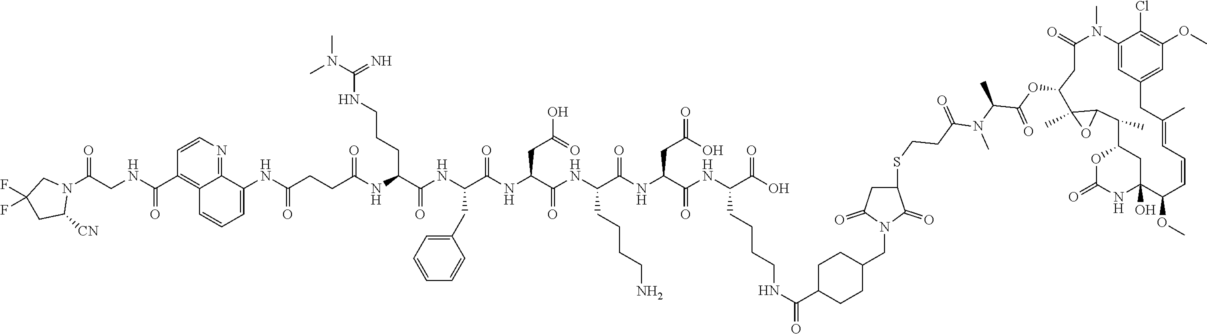 [SMILES: COc1cc2cc(c1Cl)N(C)C(=O)C[C@@H](OC(=O)[C@H](C)N(C)C(=O)CCSC1CC(=O)N(CC3CCC(C(=O)NCCCC[C@H](NC(=O)[C@H](CC(=O)O)NC(=O)[C@H](CCCCN)NC(=O)[C@H](CC(=O)O)NC(=O)[C@H](Cc4ccccc4)NC(=O)[C@H](CCCNC(=N)N(C)C)NC(=O)CCC(=O)Nc4cccc5c(C(=O)NCC(=O)N6CC(F)(F)C[C@H]6C#N)ccnc45)C(=O)O)CC3)C1=O)[C@]1(C)OC1[C@H](C)[C@@H]1C[C@@](O)(NC(=O)O1)[C@H](OC)/C=C\C=C(/C)C2] | | |
| C26 | 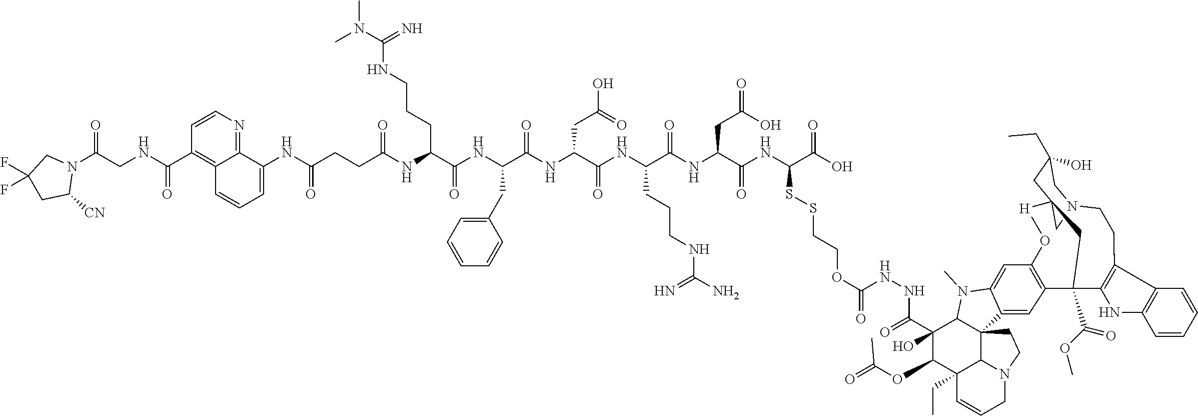 | | |
| C26a | 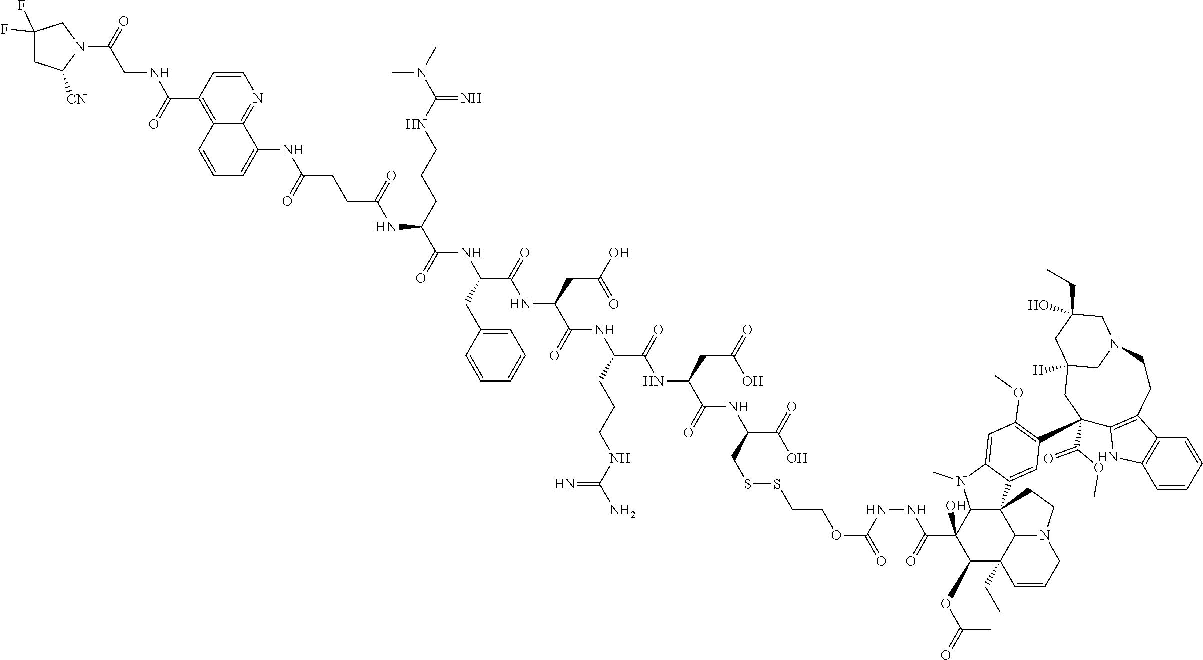 | | |
| C27 | 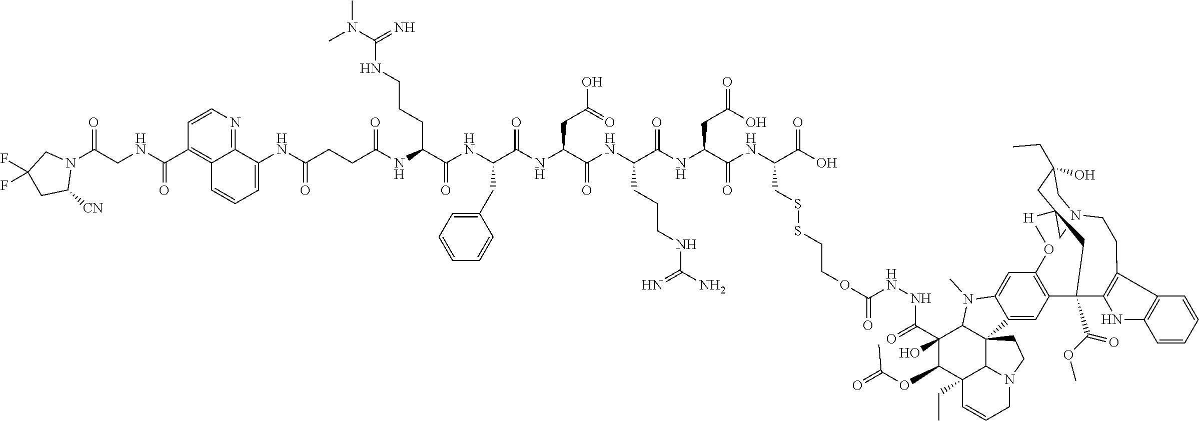 | | |
| | 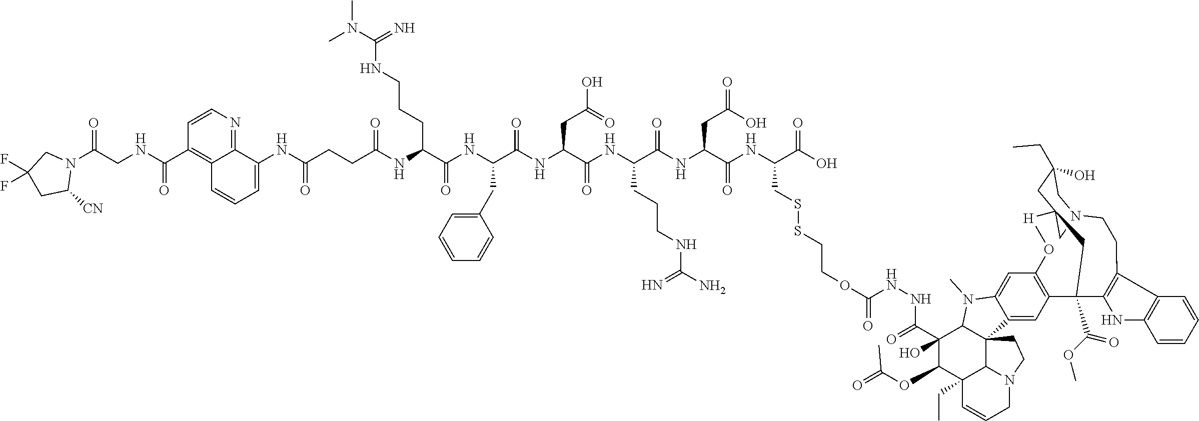 | | |
| C28 | 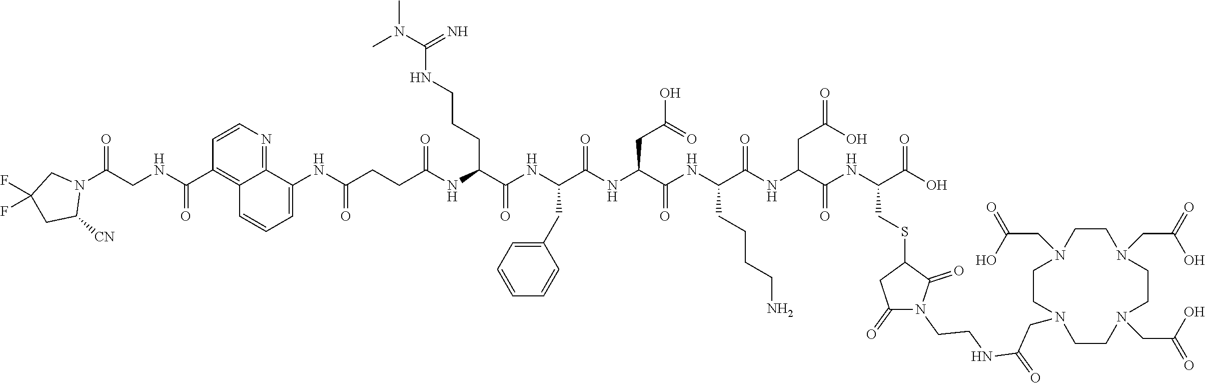 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)N[C@@H](CC(=O)O)C(=O)N[C@@H](CCCCN)C(=O)NC(CC(=O)O)C(=O)N[C@@H](CSC1CC(=O)N(CCNC(=O)CN2CCN(CC(=O)O)CCN(CC(=O)O)CCN(CC(=O)O)CC2)C1=O)C(=O)O] | | |
| C29 | 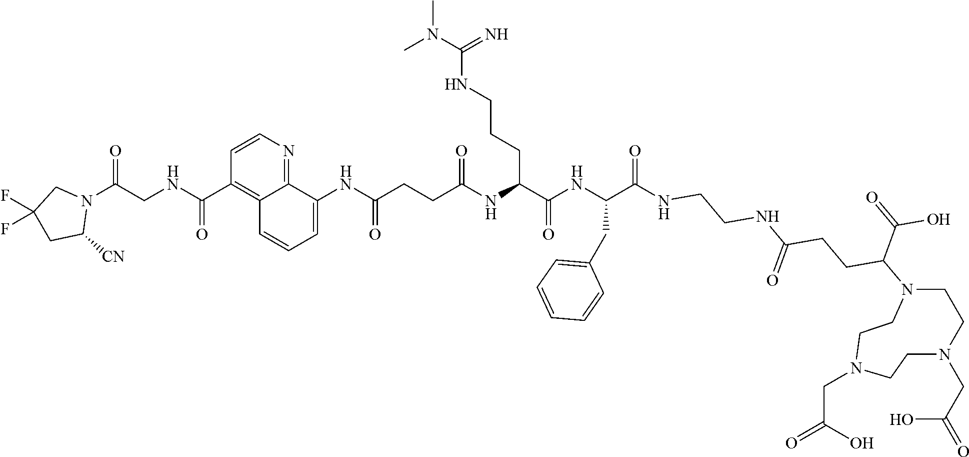 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCNC(=O)CCC(C(=O)O)N1CCN(CC(=O)O)CCN(CC(=O)O)CC1] | | |
| C30 | 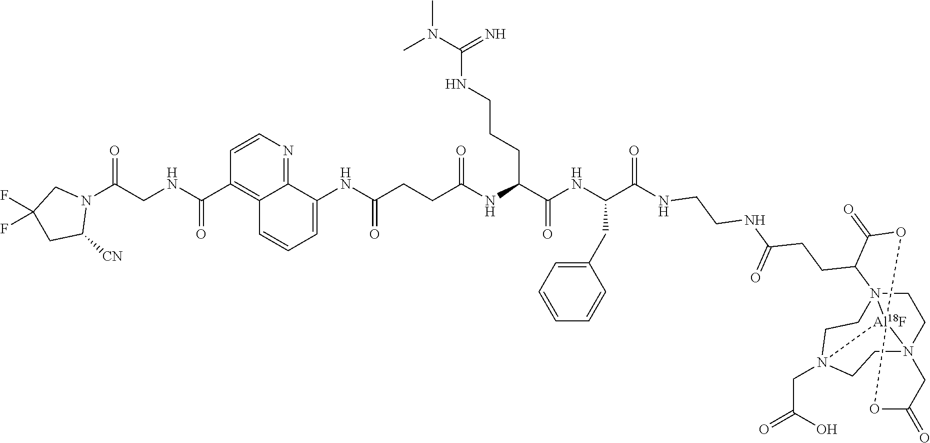 | | |
| C31 | 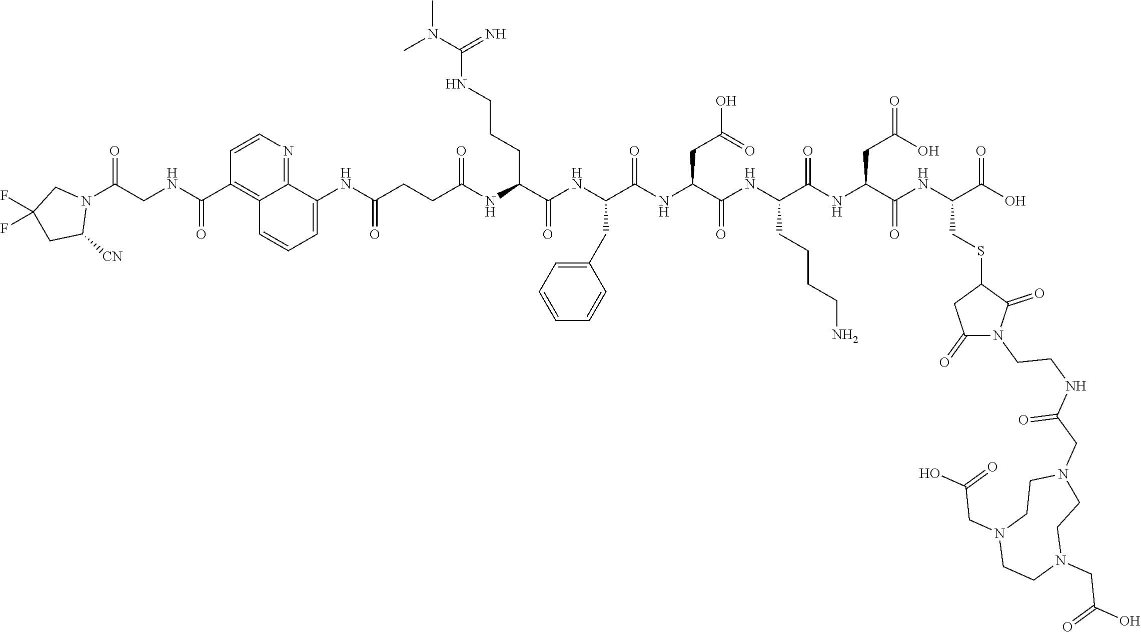 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)N[C@@H](CC(=O)O)C(=O)N[C@@H](CCCCN)C(=O)N[C@@H](CC(=O)O)C(=O)N[C@@H](CSC1CC(=O)N(CCNC(=O)CN2CCN(CC(=O)O)CCN(CC(=O)O)CC2)C1=O)C(=O)O] | | |
| C32 | 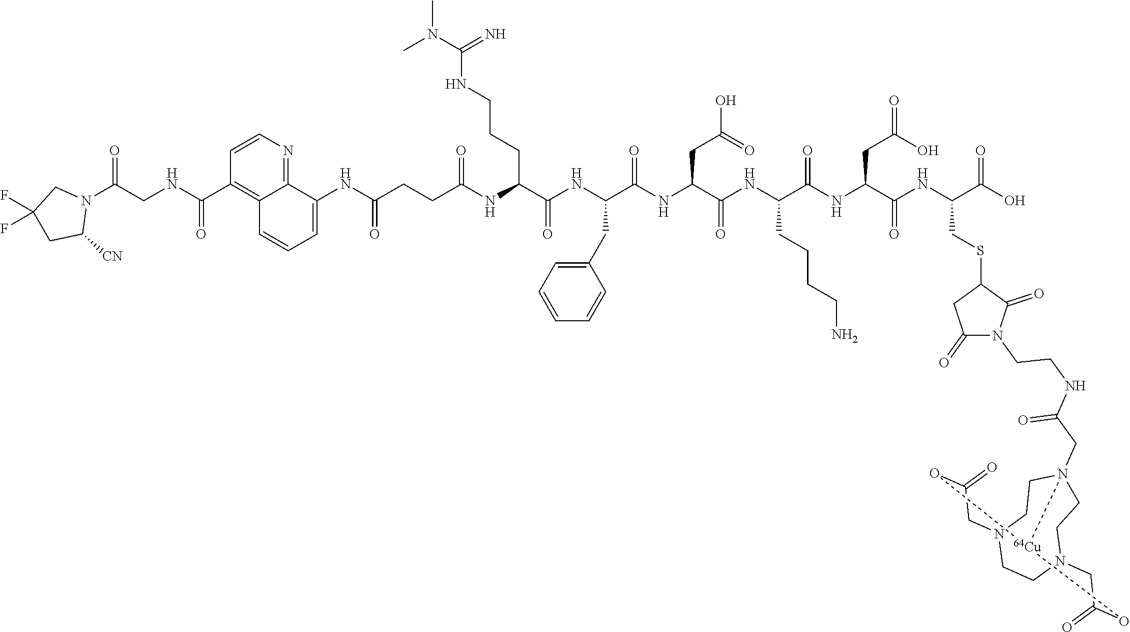 | | |
| C33 | 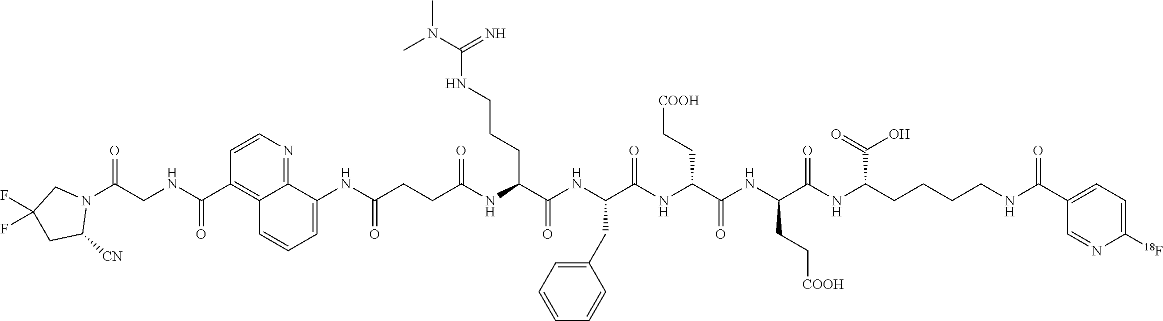 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)N[C@H](CCC(=O)O)C(=O)N[C@H](CCC(=O)O)C(=O)N[C@@H](CCCCNC(=O)c1ccc([18F])nc1)C(=O)O] | | |
| C34 | 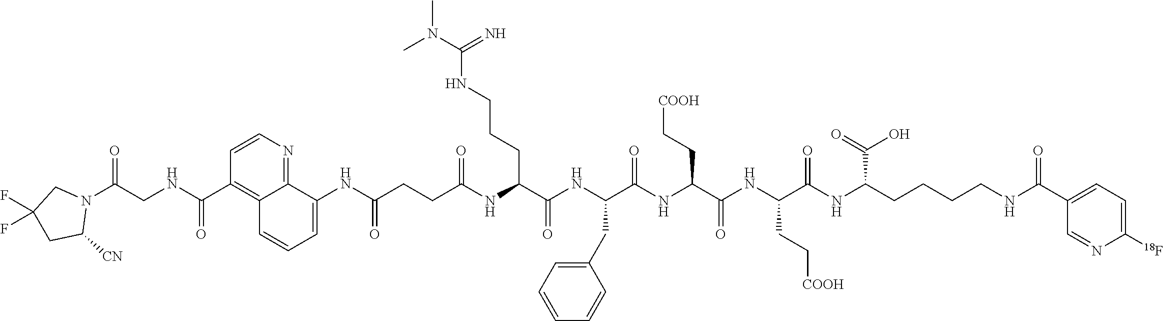 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)N[C@@H](CCC(=O)O)C(=O)N[C@@H](CCC(=O)O)C(=O)N[C@@H](CCCCNC(=O)c1ccc([18F])nc1)C(=O)O] | | |
| C35 | 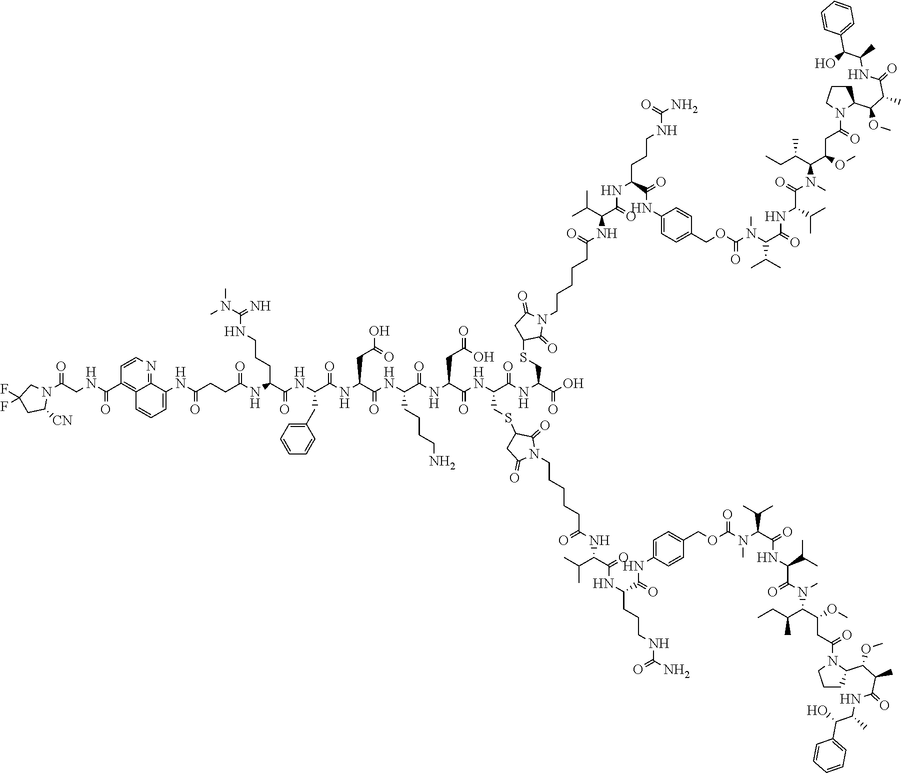 | | |
| C36 | 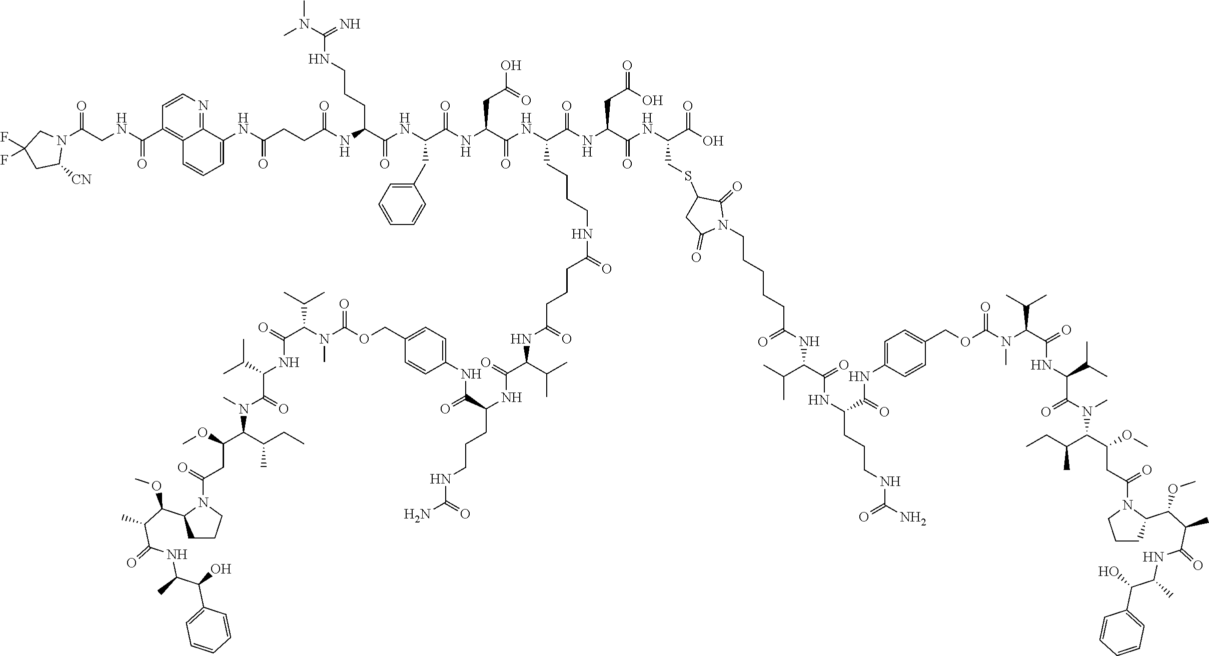 | | |
| C37 | 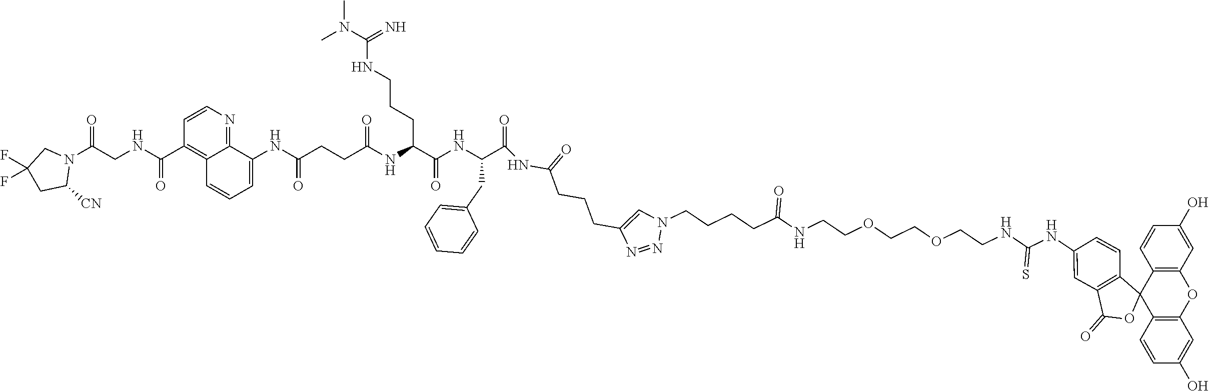 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NC(=O)CCCc1cn(CCCCC(=O)NCCOCCOCCNC(=S)Nc2ccc3c(c2)C(=O)OC32c3ccc(O)cc3Oc3cc(O)ccc32)nn1] | | |
| C38 | 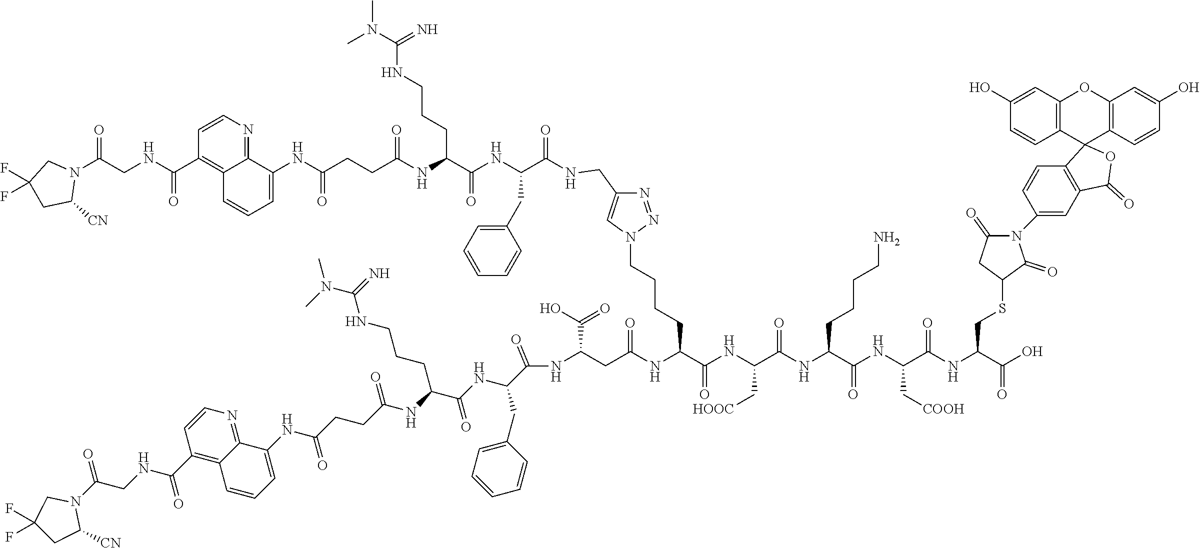 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCc1cn(CCCC[C@H](NC(=O)C[C@H](NC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)C(=O)O)C(=O)N[C@@H](CC(=O)O)C(=O)N[C@@H](CCCCN)C(=O)N[C@@H](CC(=O)O)C(=O)N[C@@H](CSC2CC(=O)N(c3ccc4c(c3)C(=O)OC43c4ccc(O)cc4Oc4cc(O)ccc43)C2=O)C(=O)O)nn1] | | |
| C39 | 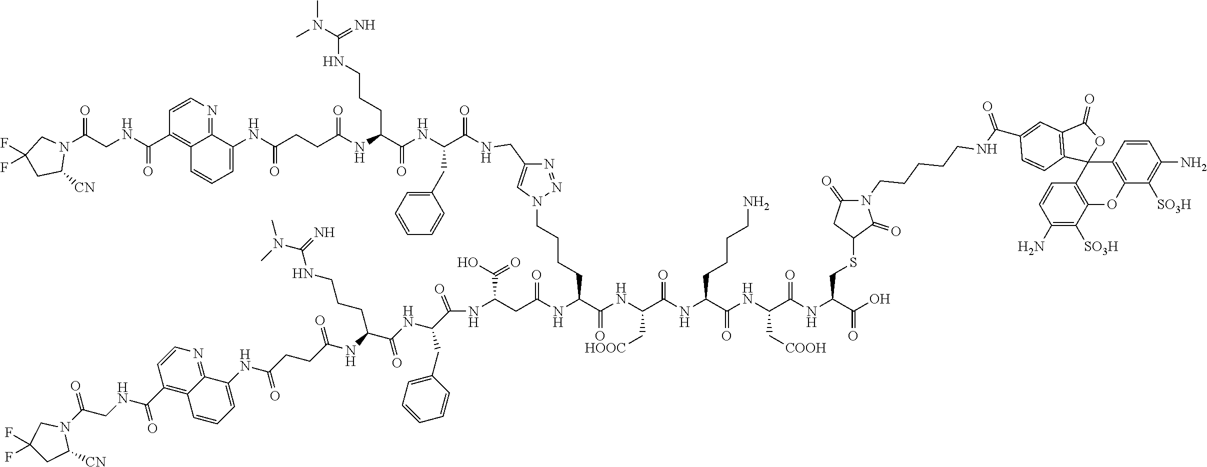 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCc1cn(CCCC[C@H](NC(=O)C[C@H](NC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)C(=O)O)C(=O)N[C@@H](CC(=O)O)C(=O)N[C@@H](CCCCN)C(=O)N[C@@H](CC(=O)O)C(=O)N[C@@H](CSC2CC(=O)N(CCCCCNC(=O)c3ccc4c(c3)C(=O)OC43c4ccc(N)c(S(=O)(=O)O)c4Oc4c3ccc(N)c4S(=O)(=O)O)C2=O)C(=O)O)nn1] | | |
| C40 | 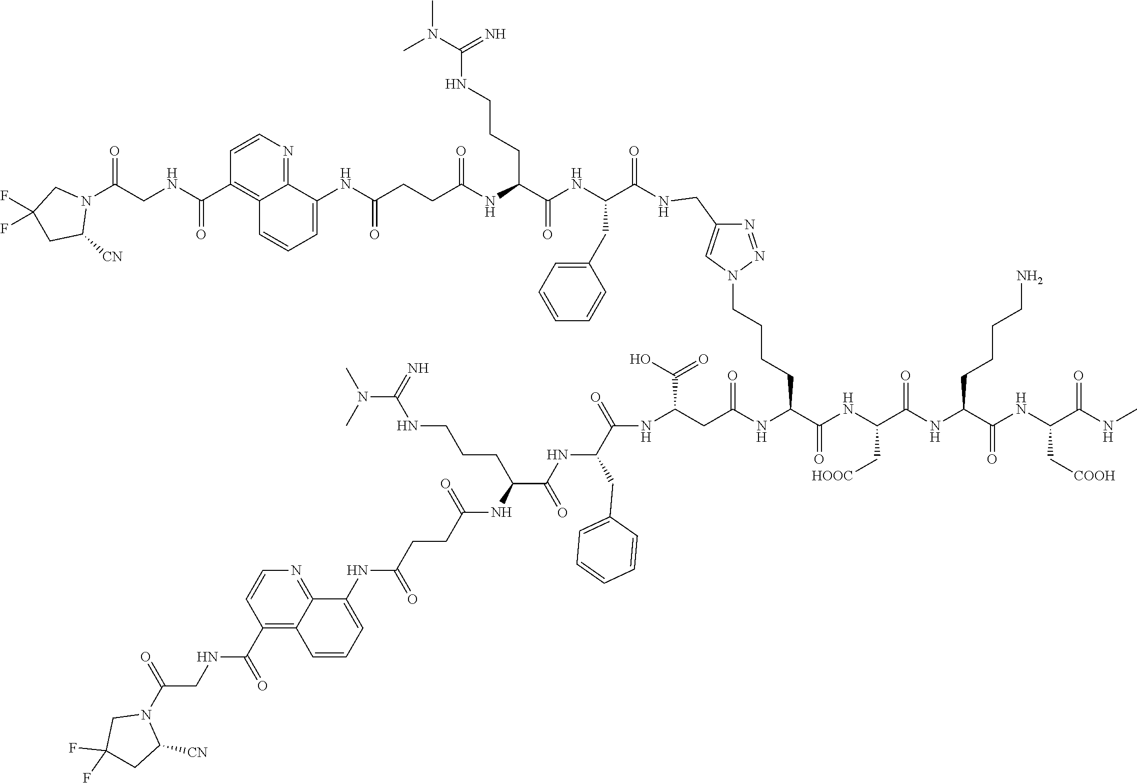 [SMILES: CNC(=O)[C@H](CC(=O)O)NC(=O)[C@H](CCCCN)NC(=O)[C@H](CC(=O)O)NC(=O)[C@H](CCCCn1cc(CNC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)nn1)NC(=O)C[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)O] 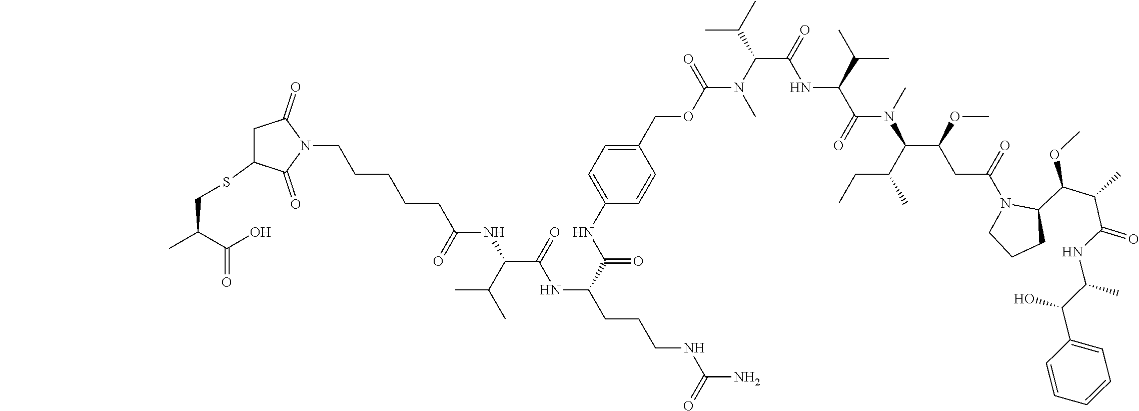 | | |
| C41 | 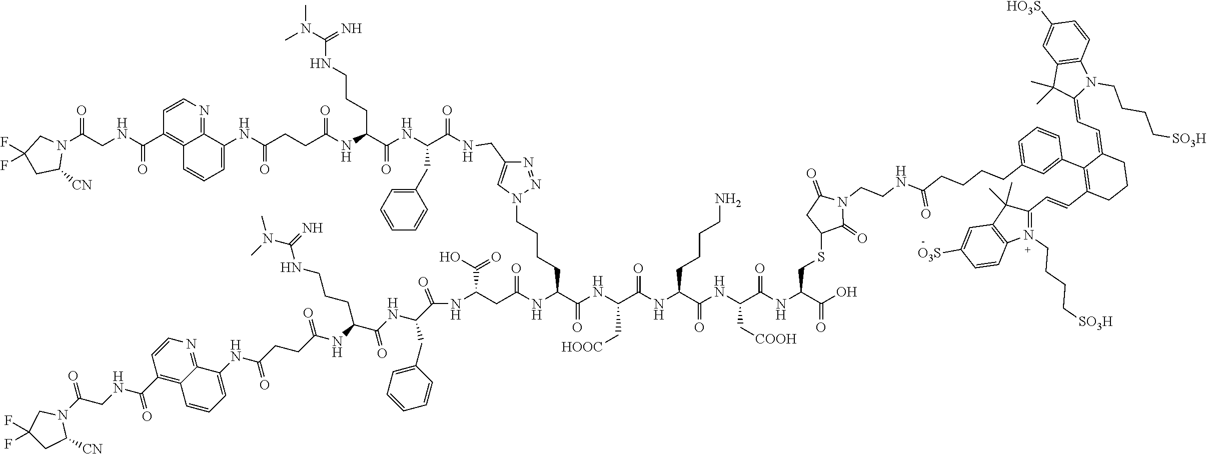 | | |
| C42 | 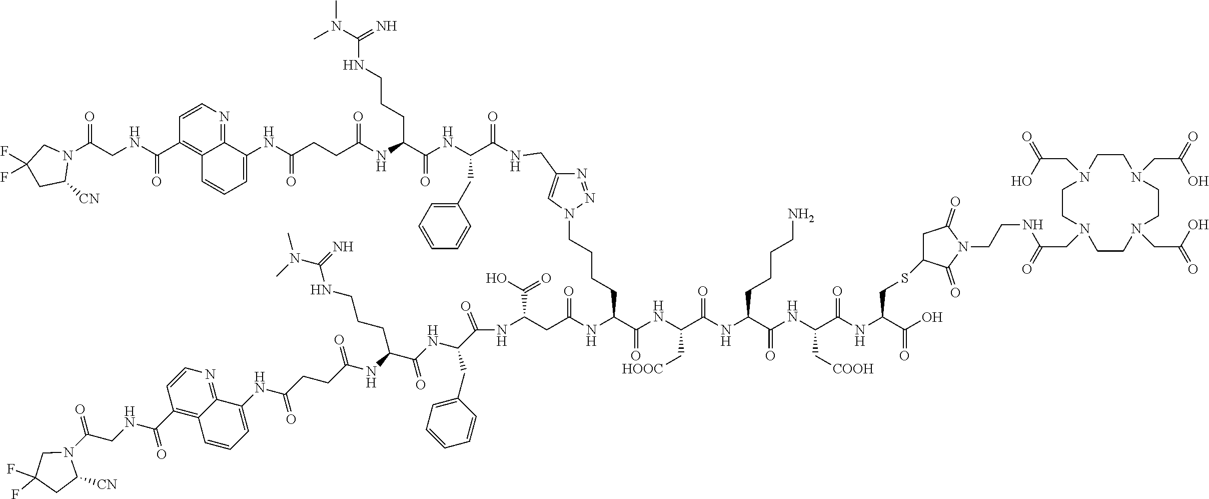 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCc1cn(CCCC[C@H](NC(=O)C[C@H](NC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)C(=O)O)C(=O)N[C@@H](CC(=O)O)C(=O)N[C@@H](CCCCN)C(=O)N[C@@H](CC(=O)O)C(=O)N[C@@H](CSC2CC(=O)N(CCNC(=O)CN3CCN(CC(=O)O)CCN(CC(=O)O)CCN(CC(=O)O)CC3)C2=O)C(=O)O)nn1] | | |
| C43 | 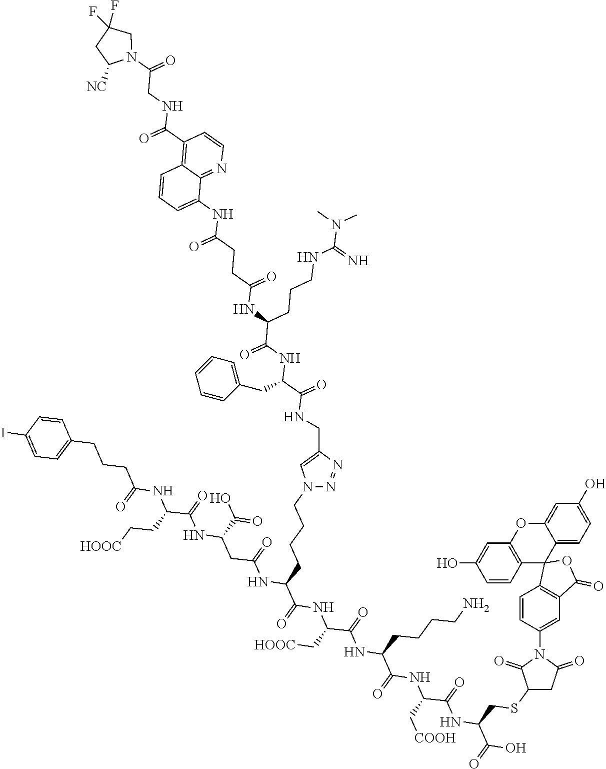 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCc1cn(CCCC[C@H](NC(=O)C[C@H](NC(=O)[C@H](CCC(=O)O)NC(=O)CCCc2ccc(I)cc2)C(=O)O)C(=O)N[C@@H](CC(=O)O)C(=O)N[C@@H](CCCCN)C(=O)N[C@@H](CC(=O)O)C(=O)N[C@@H](CSC2CC(=O)N(c3ccc4c(c3)C(=O)OC43c4ccc(O)cc4Oc4cc(O)ccc43)C2=O)C(=O)O)nn1] | | |
| C44 | 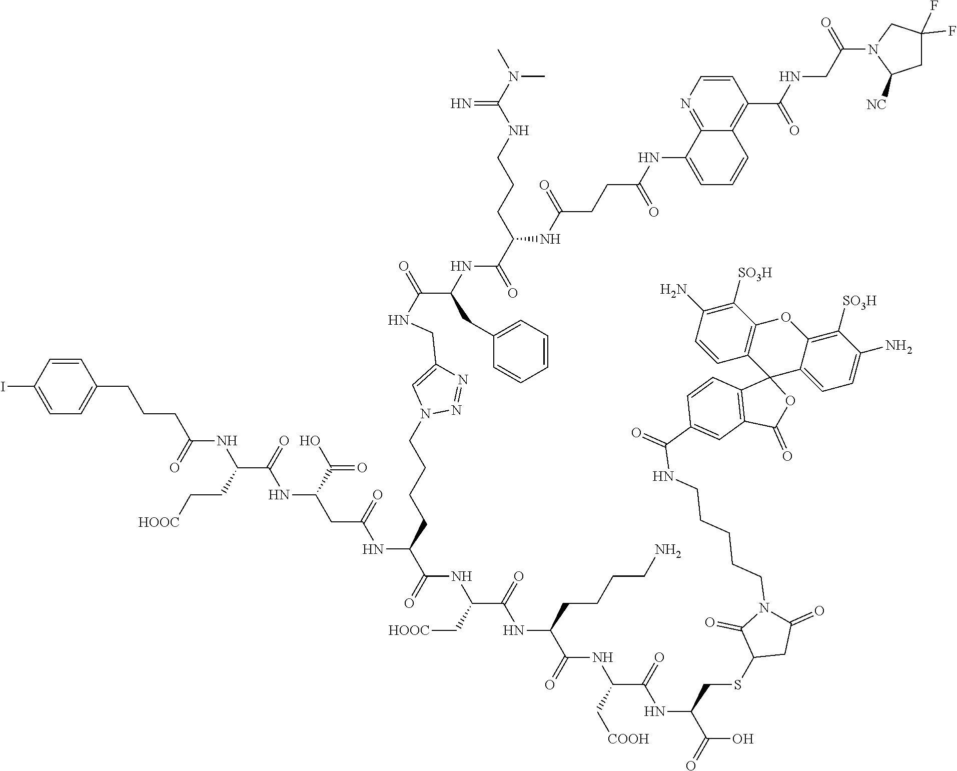 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCc1cn(CCCC[C@H](NC(=O)C[C@H](NC(=O)[C@H](CCC(=O)O)NC(=O)CCCc2ccc(I)cc2)C(=O)O)C(=O)N[C@@H](CC(=O)O)C(=O)N[C@@H](CCCCN)C(=O)N[C@@H](CC(=O)O)C(=O)N[C@@H](CSC2CC(=O)N(CCCCCNC(=O)c3ccc4c(c3)C(=O)OC43c4ccc(N)c(S(=O)(=O)O)c4Oc4c3ccc(N)c4S(=O)(=O)O)C2=O)C(=O)O)nn1] | | |
| C45 | 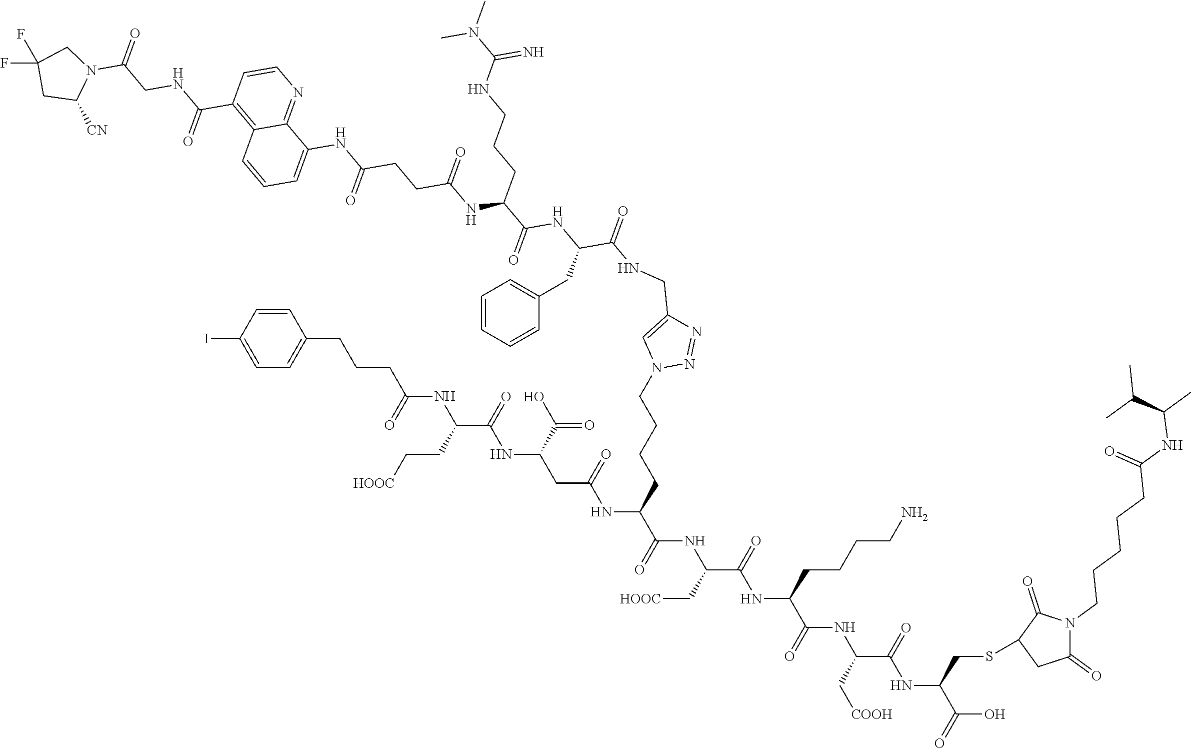 [SMILES: CC(C)[C@@H](C)NC(=O)CCCCCN1C(=O)CC(SC[C@H](NC(=O)[C@H](CC(=O)O)NC(=O)[C@H](CCCCN)NC(=O)[C@H](CC(=O)O)NC(=O)[C@H](CCCCn2cc(CNC(=O)[C@H](Cc3ccccc3)NC(=O)[C@H](CCCNC(=N)N(C)C)NC(=O)CCC(=O)Nc3cccc4c(C(=O)NCC(=O)N5CC(F)(F)C[C@H]5C#N)ccnc34)nn2)NC(=O)C[C@H](NC(=O)[C@H](CCC(=O)O)NC(=O)CCCc2ccc(I)cc2)C(=O)O)C(=O)O)C1=O] 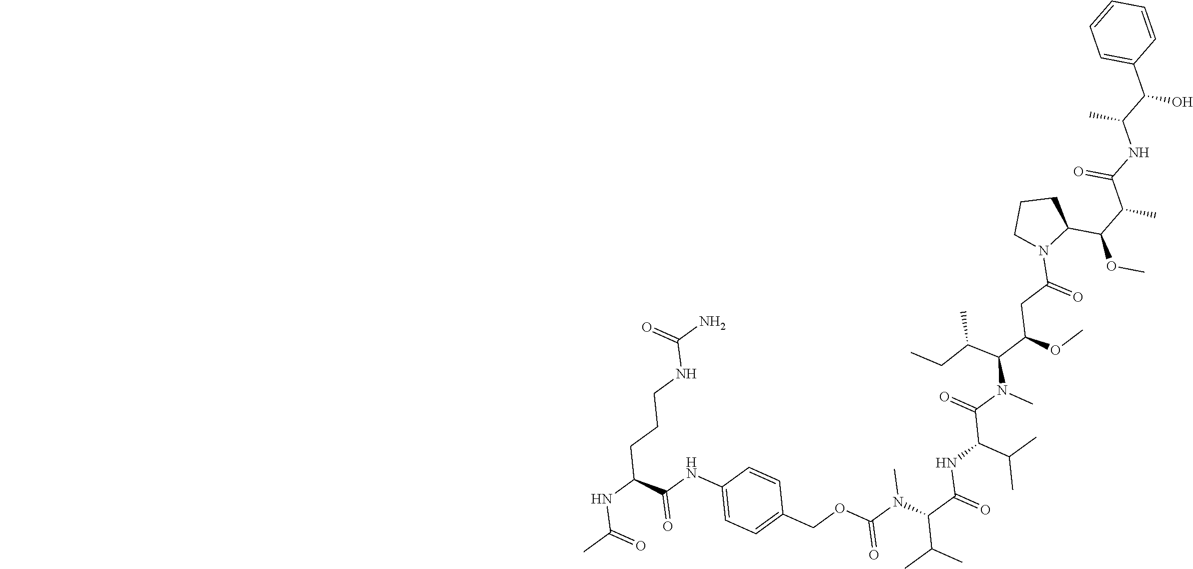 | | |
| C46 | 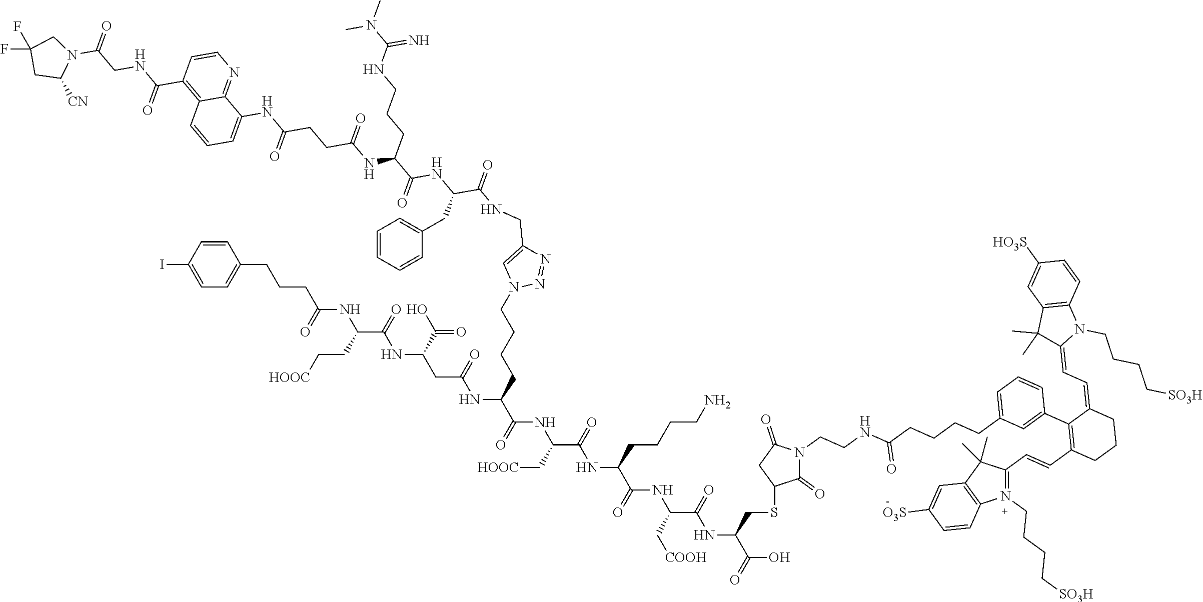 | | |
| C47 | 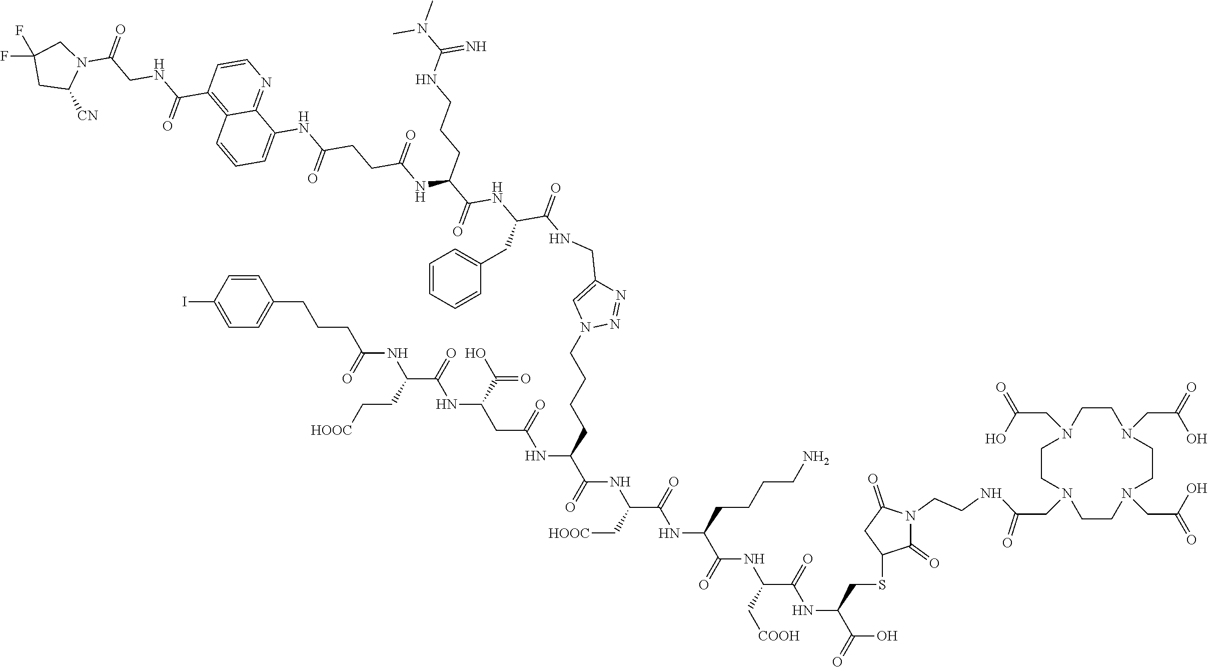 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCc1cn(CCCC[C@H](NC(=O)C[C@H](NC(=O)[C@H](CCC(=O)O)NC(=O)CCCc2ccc(I)cc2)C(=O)O)C(=O)N[C@@H](CC(=O)O)C(=O)N[C@@H](CCCCN)C(=O)N[C@@H](CC(=O)O)C(=O)N[C@@H](CSC2CC(=O)N(CCNC(=O)CN3CCN(CC(=O)O)CCN(CC(=O)O)CCN(CC(=O)O)CC3)C2=O)C(=O)O)nn1] | | |
| C48 | 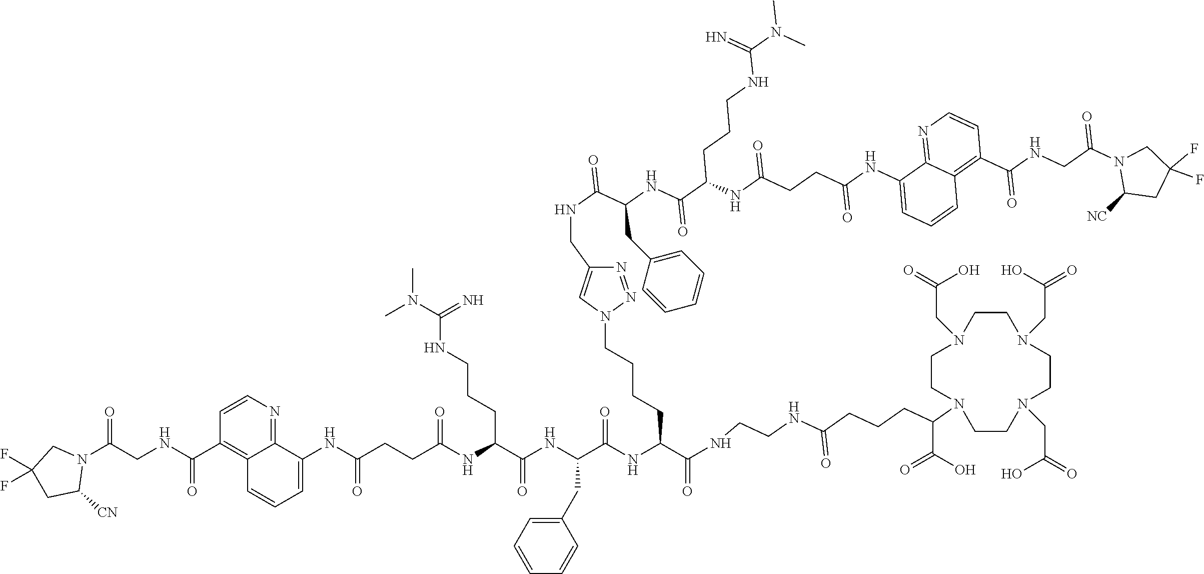 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCc1cn(CCCC[C@H](NC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)C(=O)NCCNC(=O)CCCC(C(=O)O)N2CCN(CC(=O)O)CCN(CC(=O)O)CCN(CC(=O)O)CC2)nn1] | | |
| C49 C49.1 C49.2 C49.3 C49.4 C49.5 C49.6 | R derived from AA: Glycine Alanine Valine Isoleucine Proline Arginine | | 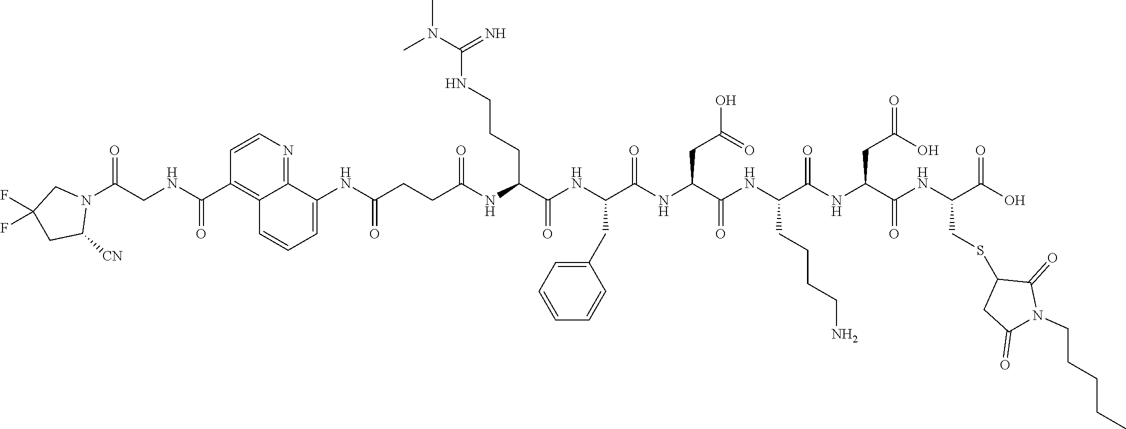 [SMILES: CCCCCN1C(=O)CC(SC[C@H](NC(=O)[C@H](CC(=O)O)NC(=O)[C@H](CCCCN)NC(=O)[C@H](CC(=O)O)NC(=O)[C@H](Cc2ccccc2)NC(=O)[C@H](CCCNC(=N)N(C)C)NC(=O)CCC(=O)Nc2cccc3c(C(=O)NCC(=O)N4CC(F)(F)C[C@H]4C#N)ccnc23)C(=O)O)C1=O] 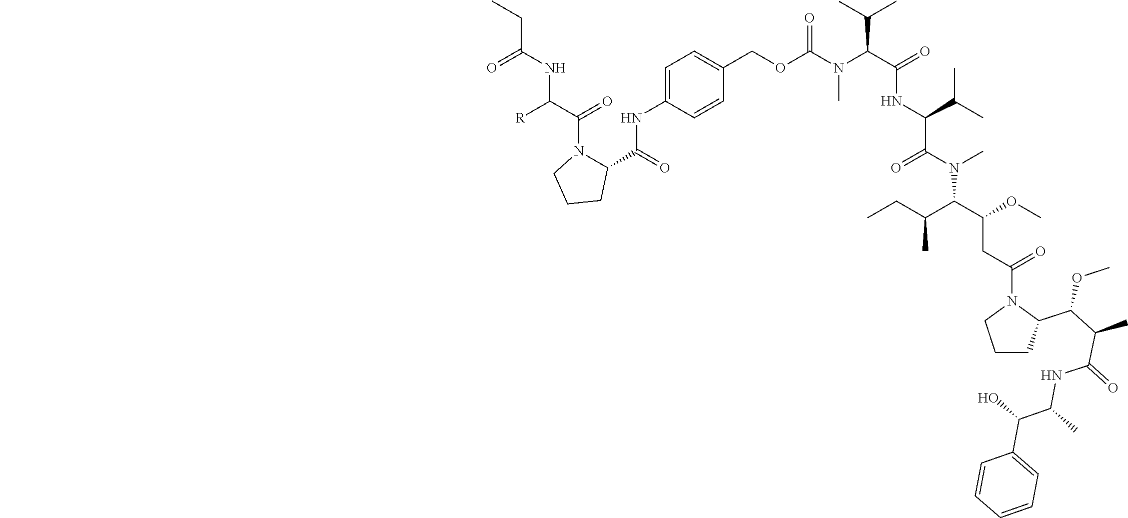 |
| C50 C50.1 C50.2 C50.3 | R H CH₃ CH₃ | R′ H H CH₃ | 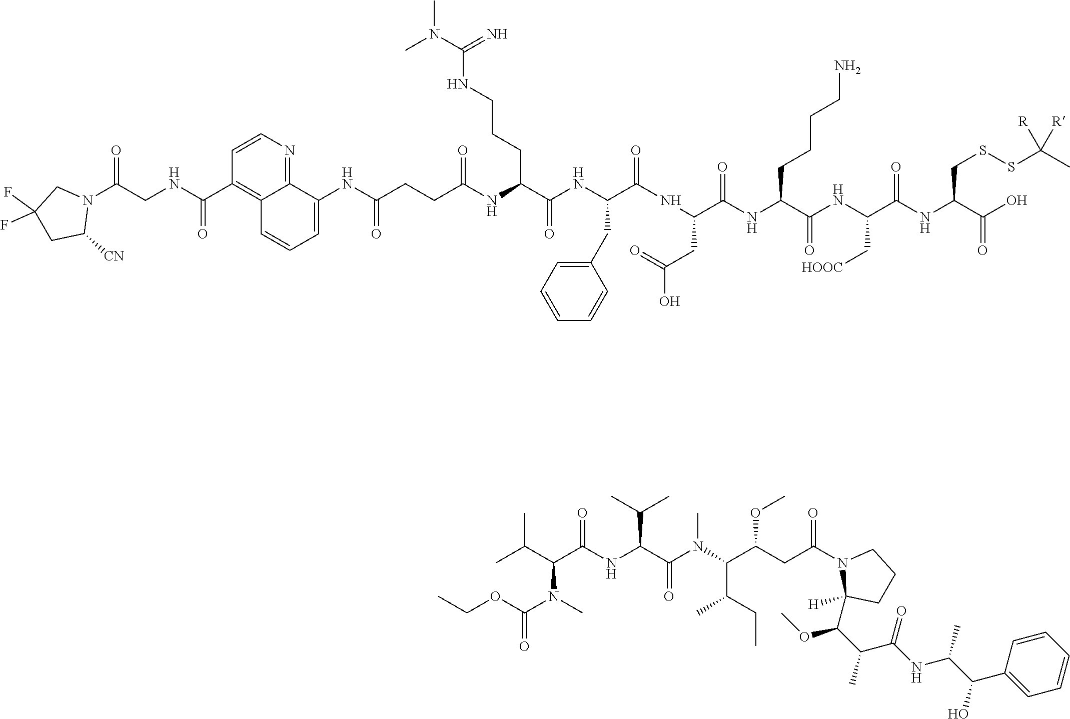 |
| C51 C51.1 C51.2 C51.3 | R H CH₃ CH₃ | R′ H H CH₃ | 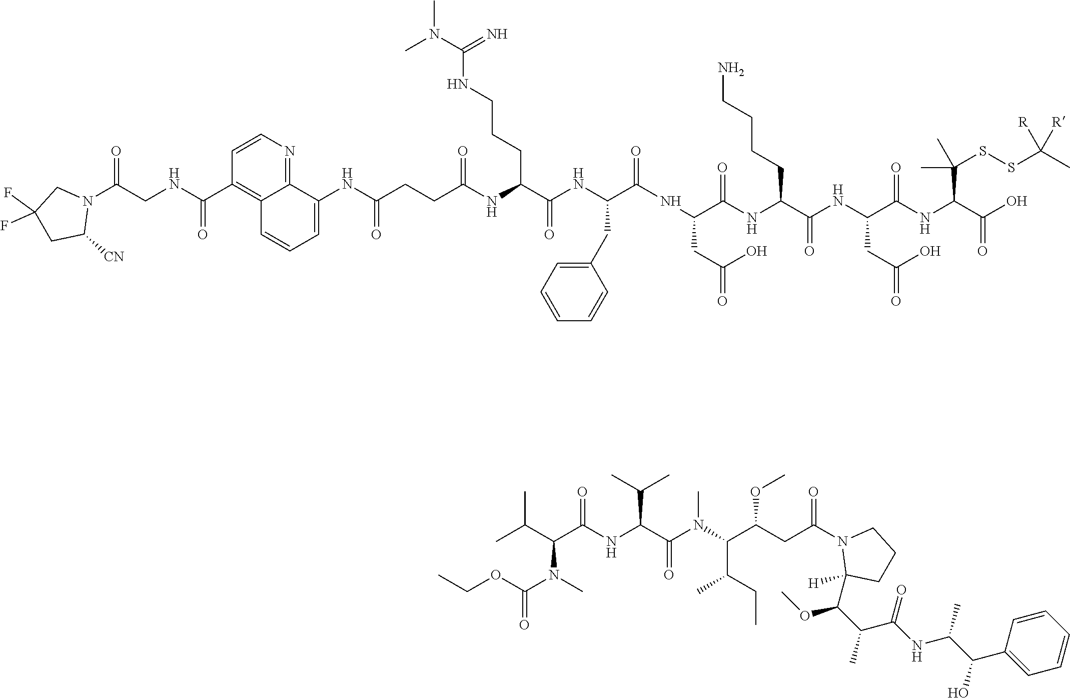 |

| | |
|---|---|
| C52 | 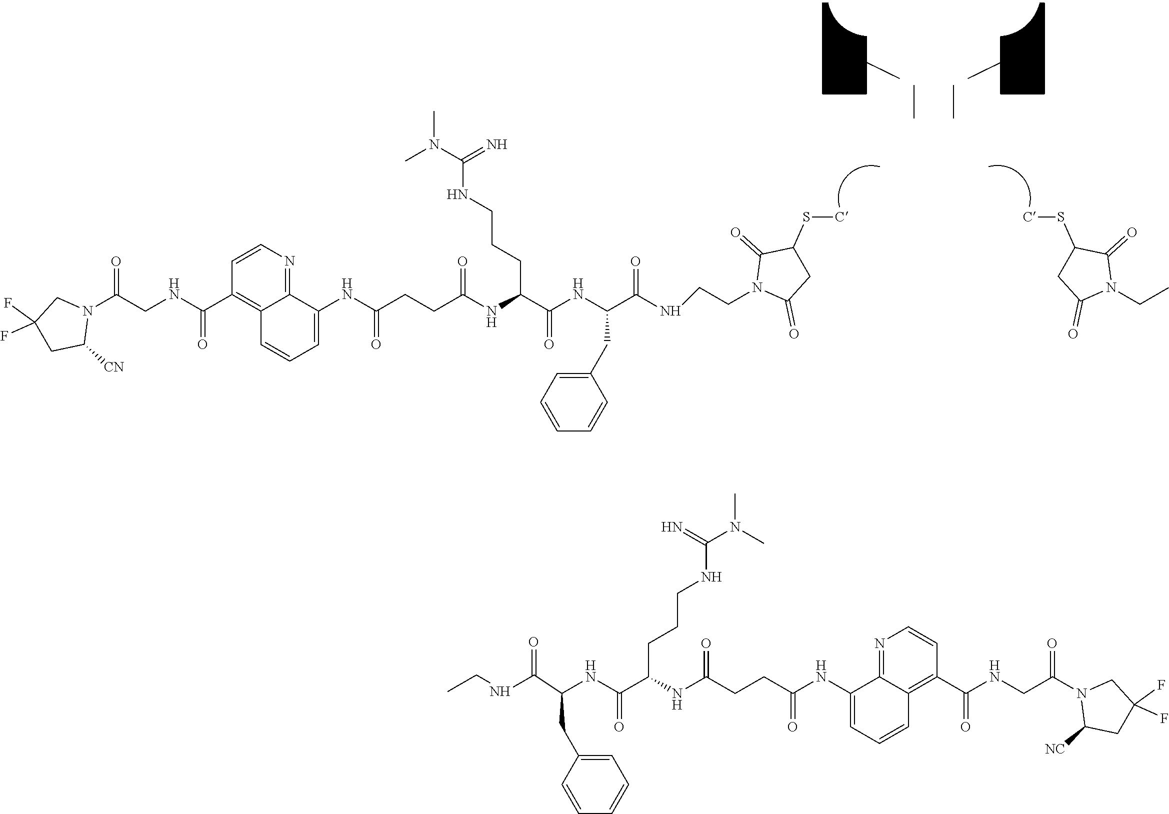 |
| | 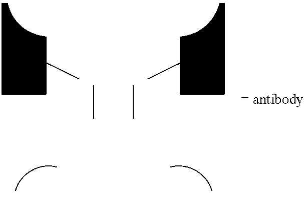 |
| C53 | 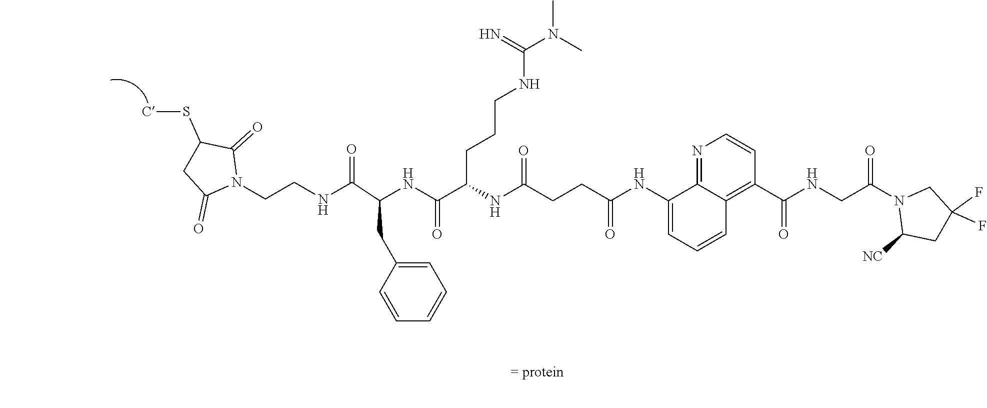 |
| C54 | 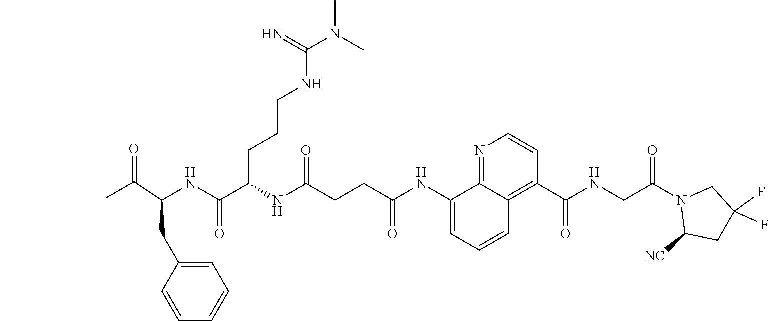 [SMILES: CC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12] |
| C55 | 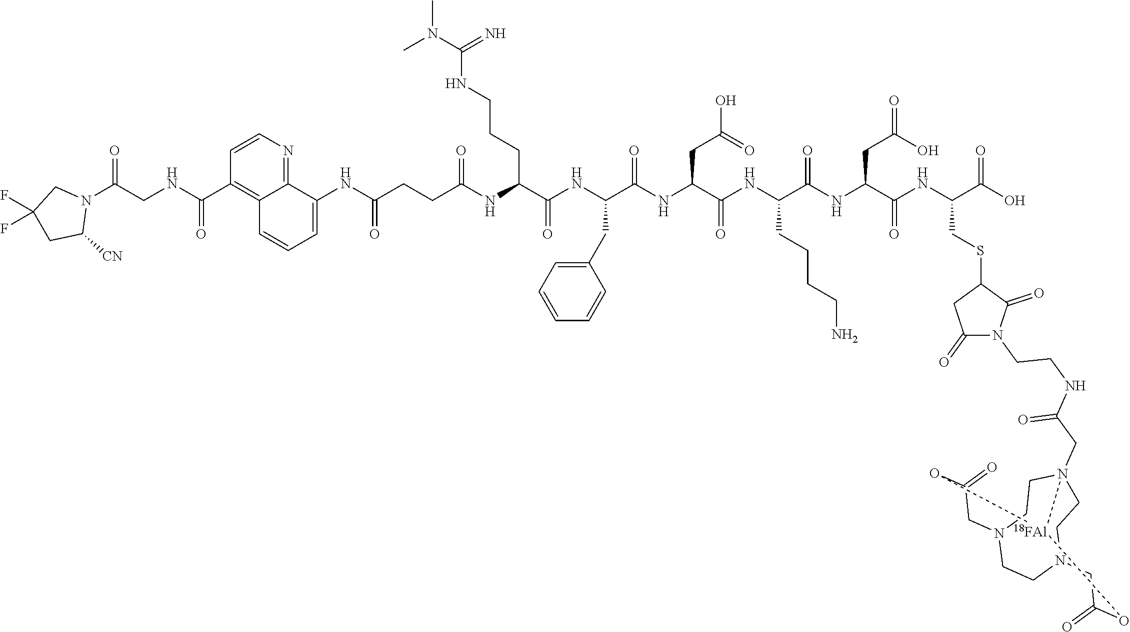 |
| C56 | 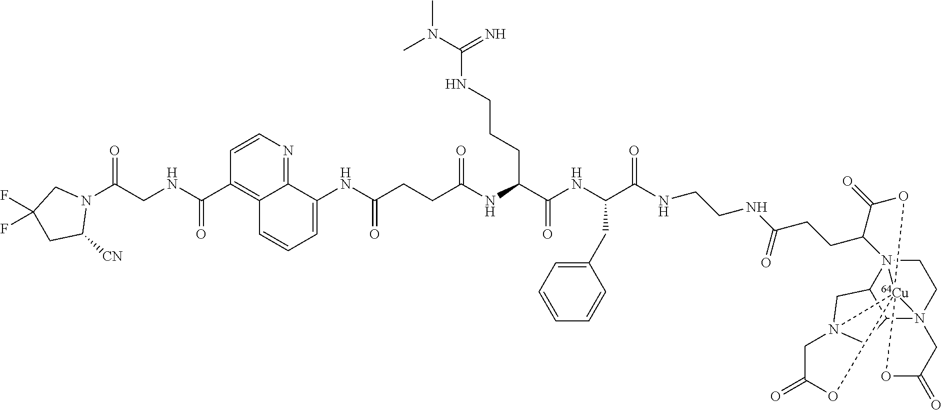 |
| C57 | 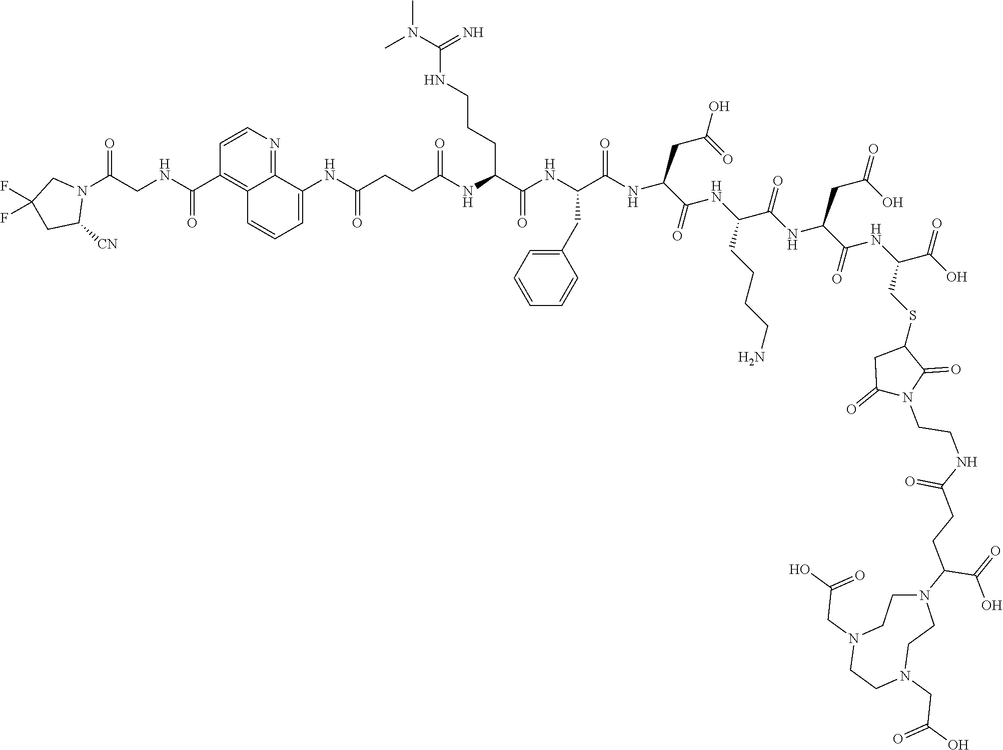 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)N[C@@H](CC(=O)O)C(=O)N[C@@H](CCCCN)C(=O)N[C@@H](CC(=O)O)C(=O)N[C@@H](CSC1CC(=O)N(CCNC(=O)CCC(C(=O)O)N2CCN(CC(=O)O)CCN(CC(=O)O)CC2)C1=O)C(=O)O] |
| C58 | 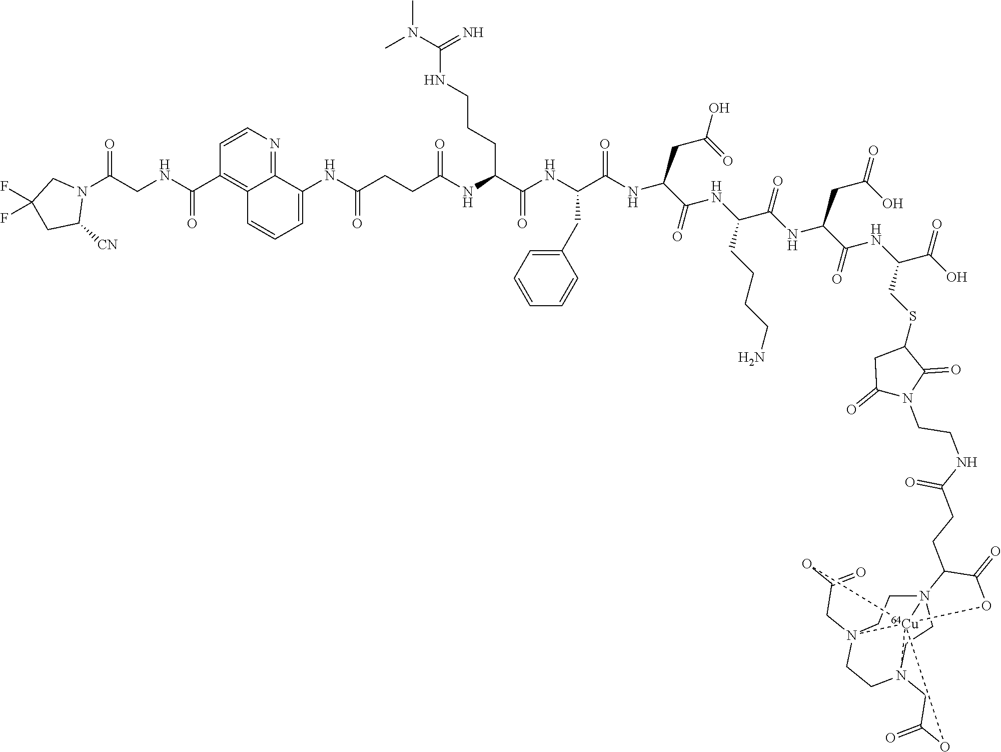 |
| C59 | 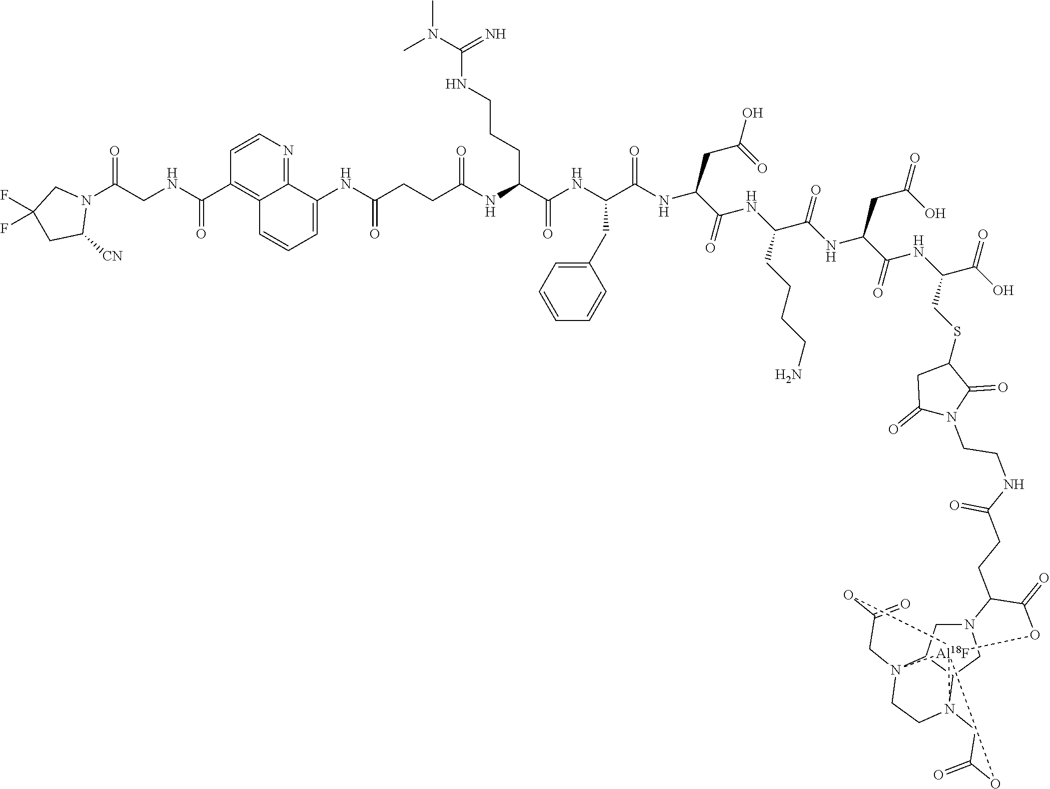 |
| C60 | 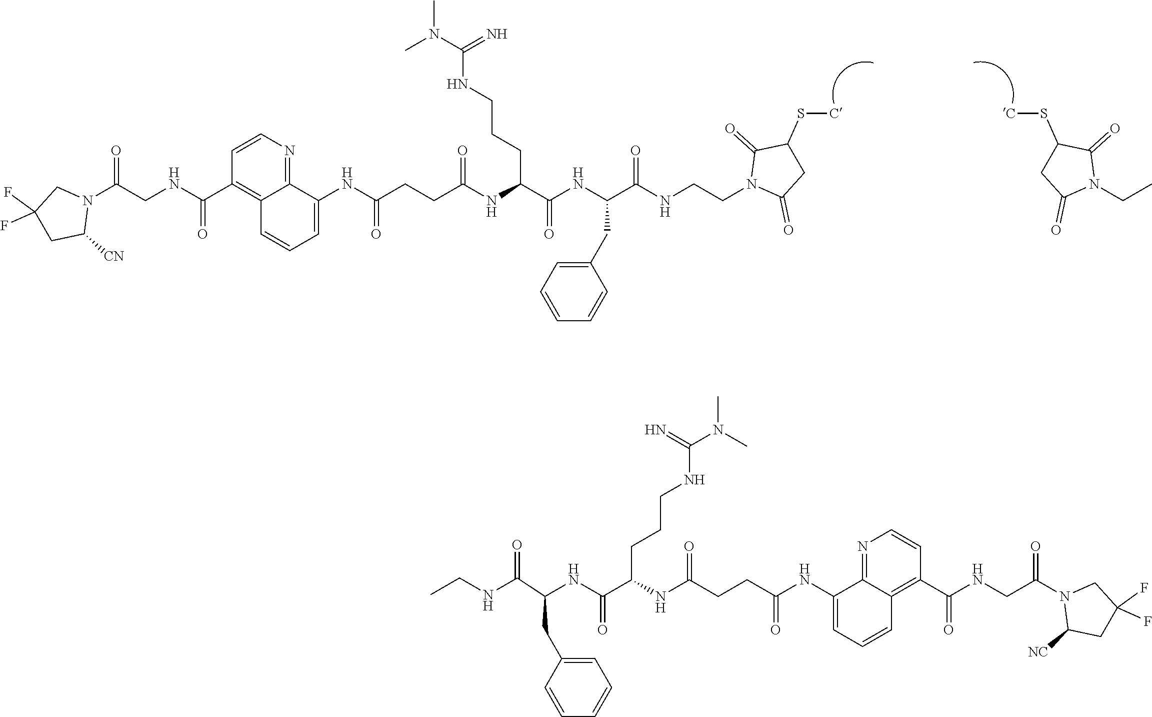 |
| C61 | 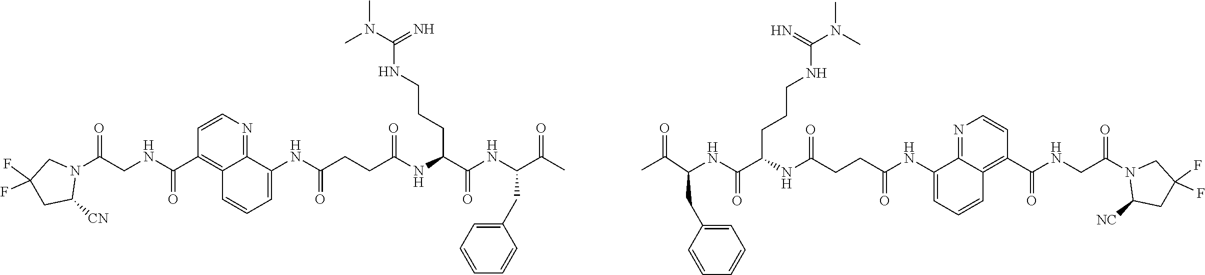 [SMILES: CC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12 ; CC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12] |
| C62 | 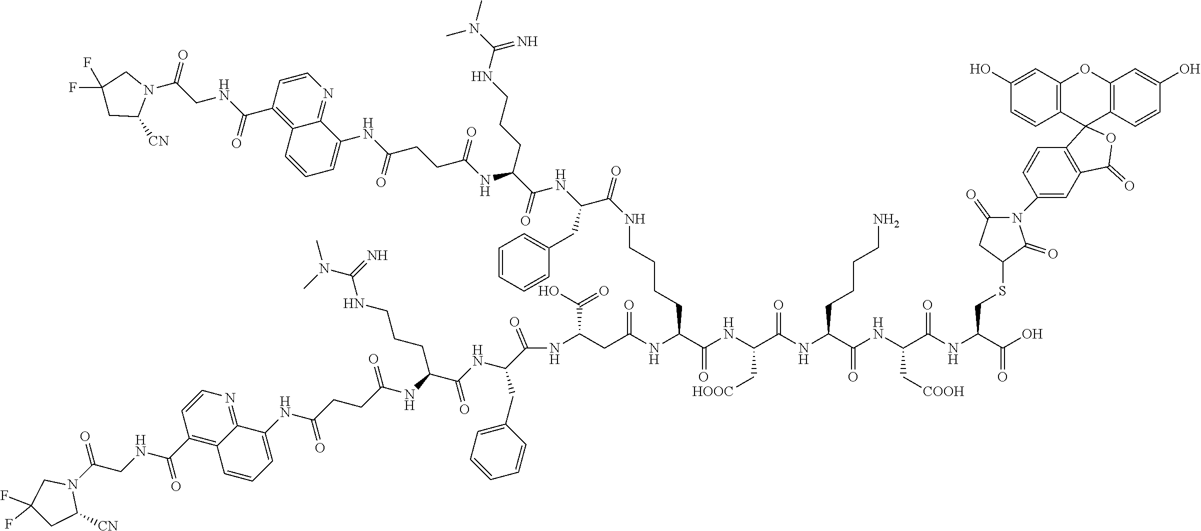 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)O)C(=O)N[C@@H](CC(=O)O)C(=O)N[C@@H](CCCCN)C(=O)N[C@@H](CC(=O)O)C(=O)N[C@@H](CSC1CC(=O)N(c2ccc3c(c2)C(=O)OC32c3ccc(O)cc3Oc3cc(O)ccc32)C1=O)C(=O)O] |
| C63 | 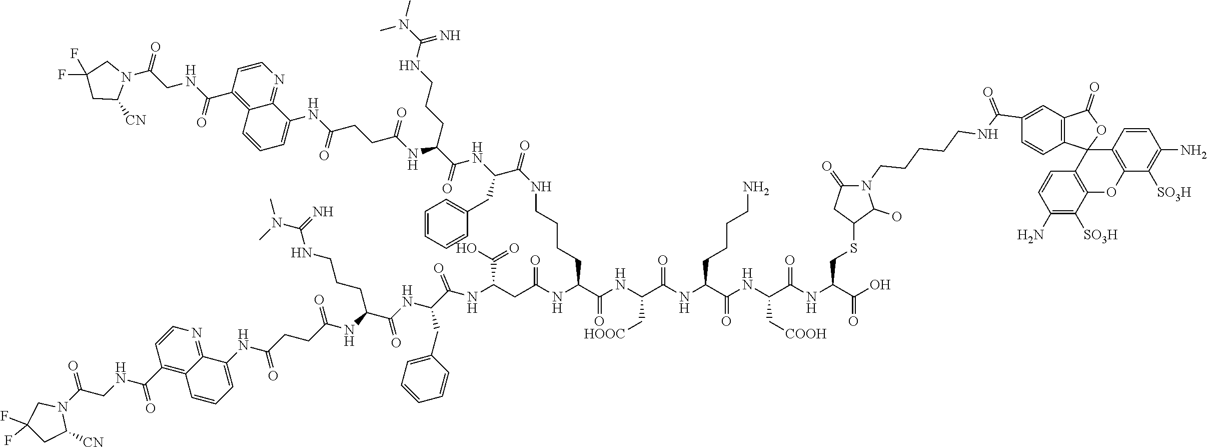 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)O)C(=O)N[C@@H](CC(=O)O)C(=O)N[C@@H](CCCCN)C(=O)N[C@@H](CC(=O)O)C(=O)N[C@@H](CSC1CC(=O)N(CCCCCNC(=O)c2ccc3c(c2)C(=O)OC32c3ccc(N)c(S(=O)(=O)O)c3Oc3c2ccc(N)c3S(=O)(=O)O)C1O)C(=O)O] |
| C64 | 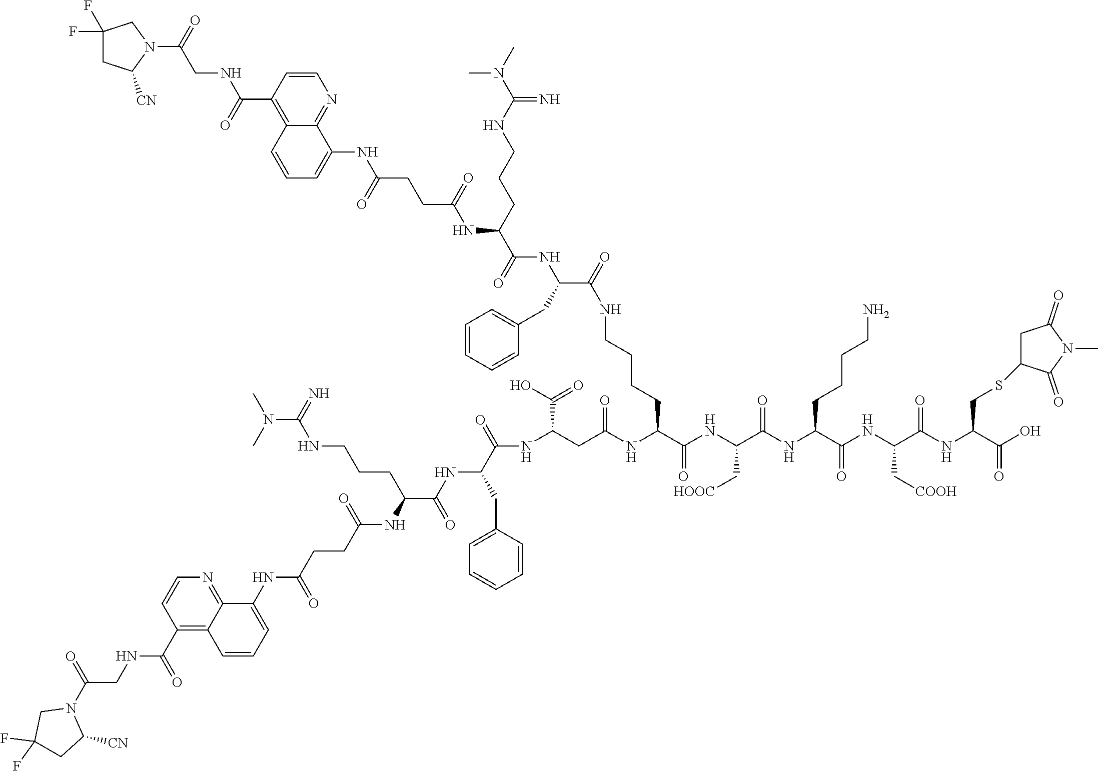 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)O)C(=O)N[C@@H](CC(=O)O)C(=O)N[C@@H](CCCCN)C(=O)N[C@@H](CC(=O)O)C(=O)N[C@@H](CSC1CC(=O)N(C)C1=O)C(=O)O] 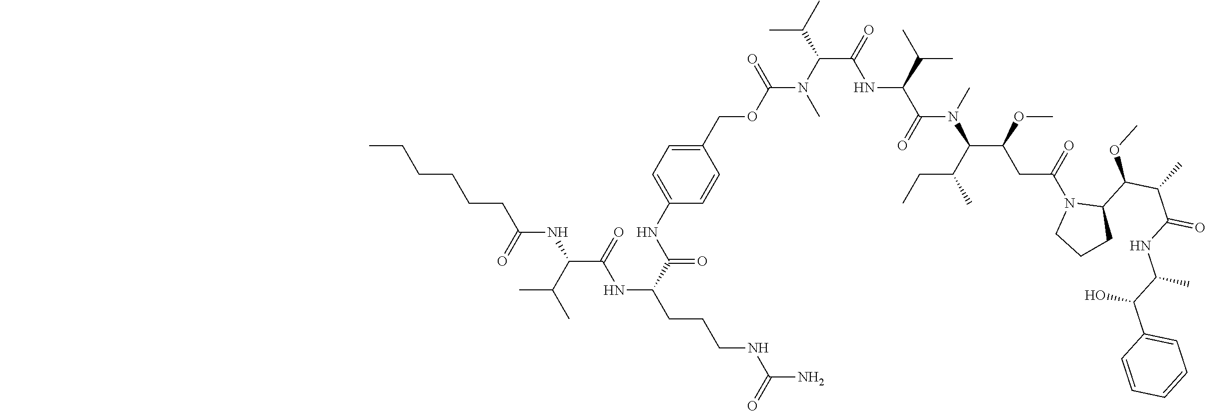 |
| C65 | 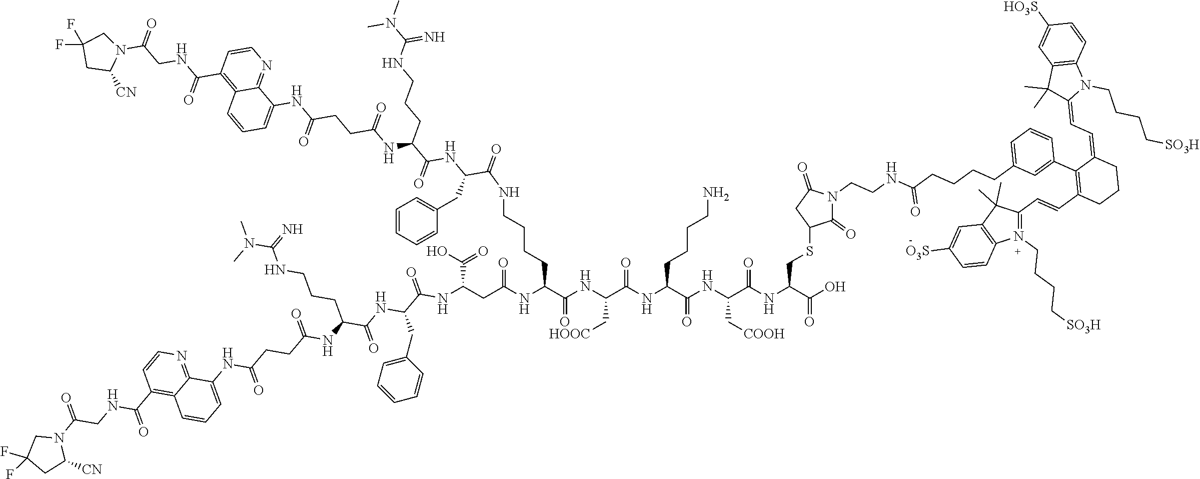 |
| C66 | 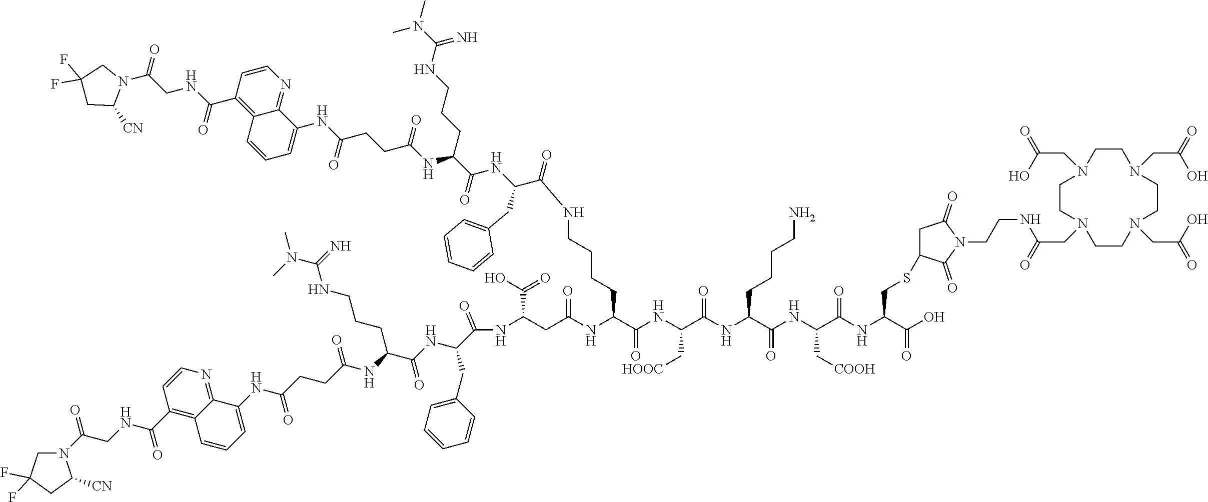 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)O)C(=O)N[C@@H](CC(=O)O)C(=O)N[C@@H](CCCCN)C(=O)N[C@@H](CC(=O)O)C(=O)N[C@@H](CSC1CC(=O)N(CCNC(=O)CN2CCN(CC(=O)O)CCN(CC(=O)O)CCN(CC(=O)O)CC2)C1=O)C(=O)O] |
| C67 | 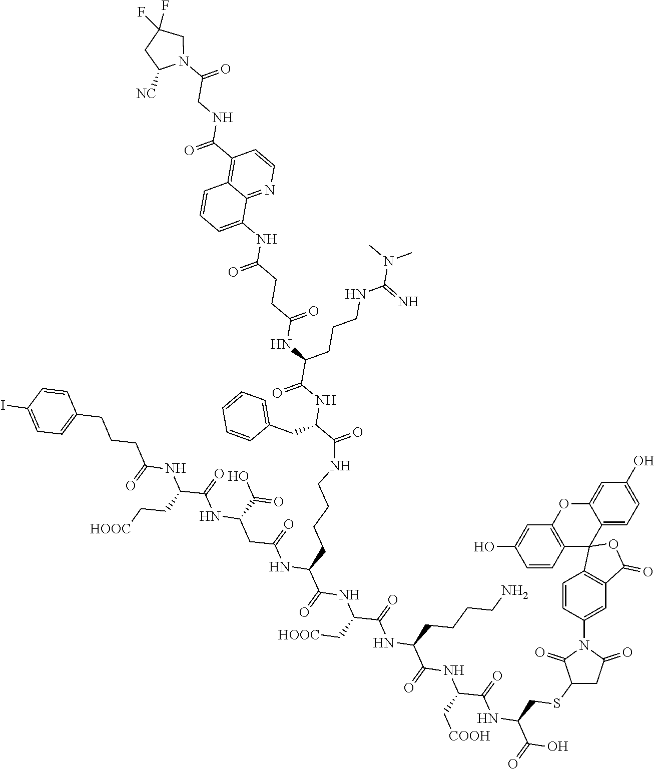 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)[C@H](CCC(=O)O)NC(=O)CCCc1ccc(I)cc1)C(=O)O)C(=O)N[C@@H](CC(=O)O)C(=O)N[C@@H](CCCCN)C(=O)N[C@@H](CC(=O)O)C(=O)N[C@@H](CSC1CC(=O)N(c2ccc3c(c2)C(=O)OC32c3ccc(O)cc3Oc3cc(O)ccc32)C1=O)C(=O)O] |
| C68 | 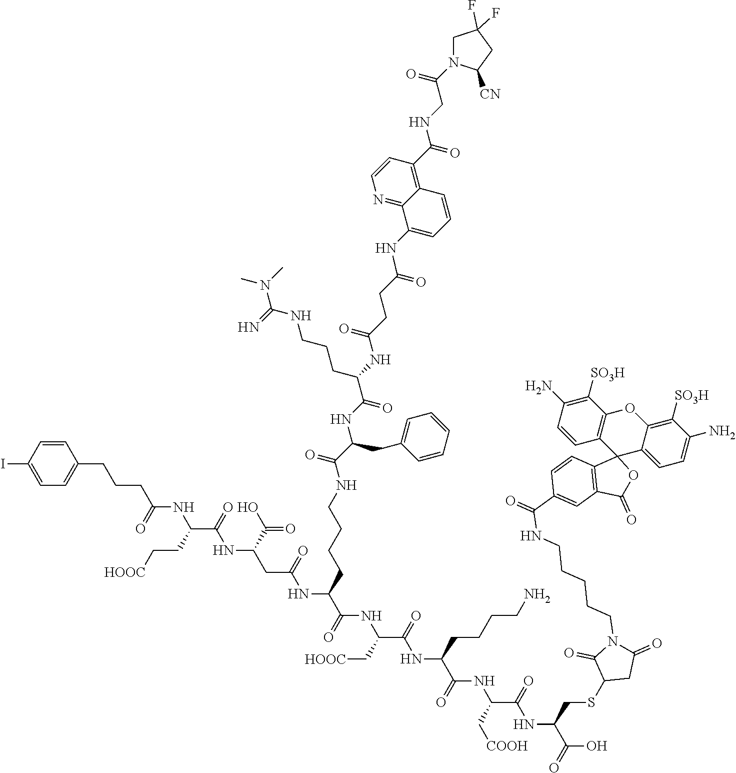 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)[C@H](CCC(=O)O)NC(=O)CCCc1ccc(I)cc1)C(=O)O)C(=O)N[C@@H](CC(=O)O)C(=O)N[C@@H](CCCCN)C(=O)N[C@@H](CC(=O)O)C(=O)N[C@@H](CSC1CC(=O)N(CCCCCNC(=O)c2ccc3c(c2)C(=O)OC32c3ccc(N)c(S(=O)(=O)O)c3Oc3c2ccc(N)c3S(=O)(=O)O)C1=O)C(=O)O] |
| C69 | 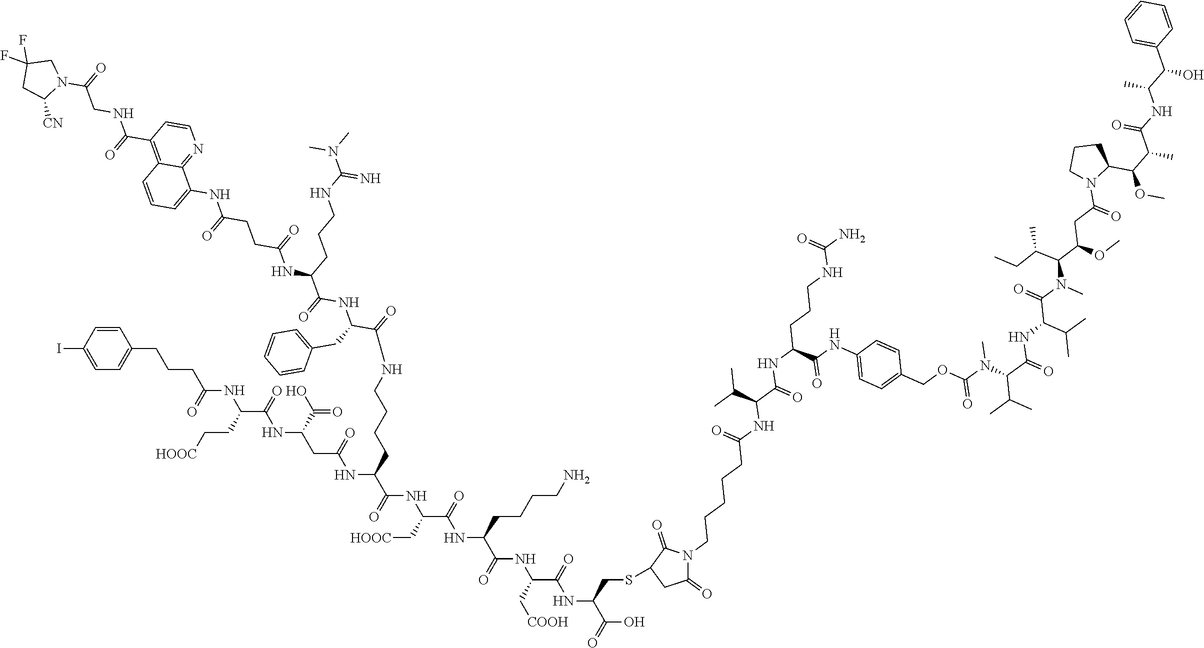 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)CC(SC[C@H](NC(=O)[C@H](CC(=O)O)NC(=O)[C@H](CCCCN)NC(=O)[C@H](CC(=O)O)NC(=O)[C@H](CCCCNC(=O)[C@H](Cc3ccccc3)NC(=O)[C@H](CCCNC(=N)N(C)C)NC(=O)CCC(=O)Nc3cccc4c(C(=O)NCC(=O)N5CC(F)(F)C[C@H]5C#N)ccnc34)NC(=O)C[C@H](NC(=O)[C@H](CCC(=O)O)NC(=O)CCCc3ccc(I)cc3)C(=O)O)C(=O)O)C2=O)C(C)C)cc1)C(C)C] |
| C70 | 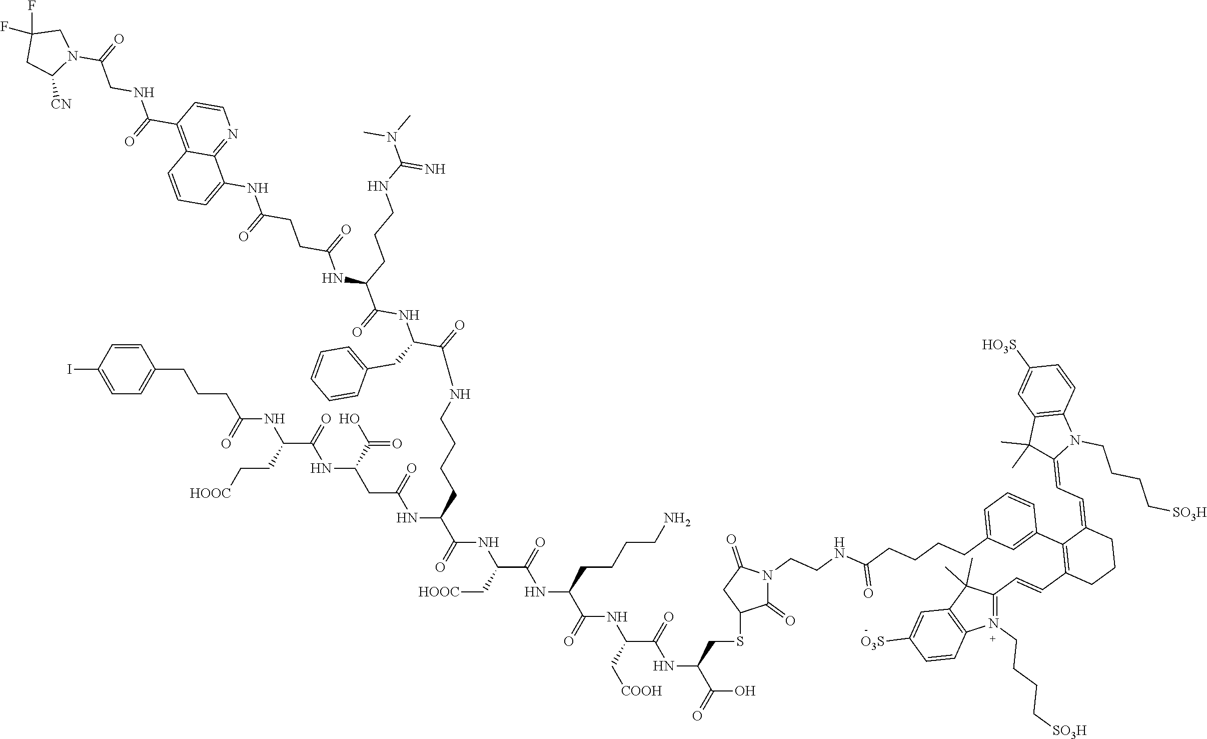 |
| C71 | 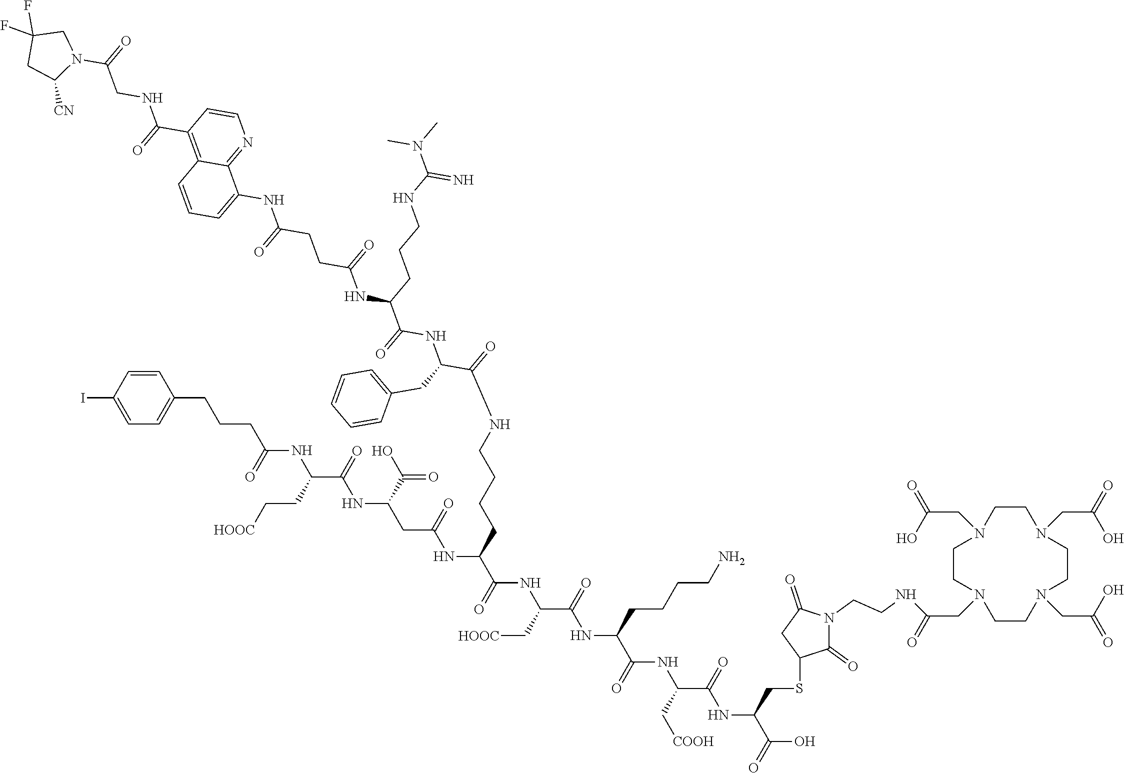 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)[C@H](CCC(=O)O)NC(=O)CCCc1ccc(I)cc1)C(=O)O)C(=O)N[C@@H](CC(=O)O)C(=O)N[C@@H](CCCCN)C(=O)N[C@@H](CC(=O)O)C(=O)N[C@@H](CSC1CC(=O)N(CCNC(=O)CN2CCN(CC(=O)O)CCN(CC(=O)O)CCN(CC(=O)O)CC2)C1=O)C(=O)O] |
| C72 | 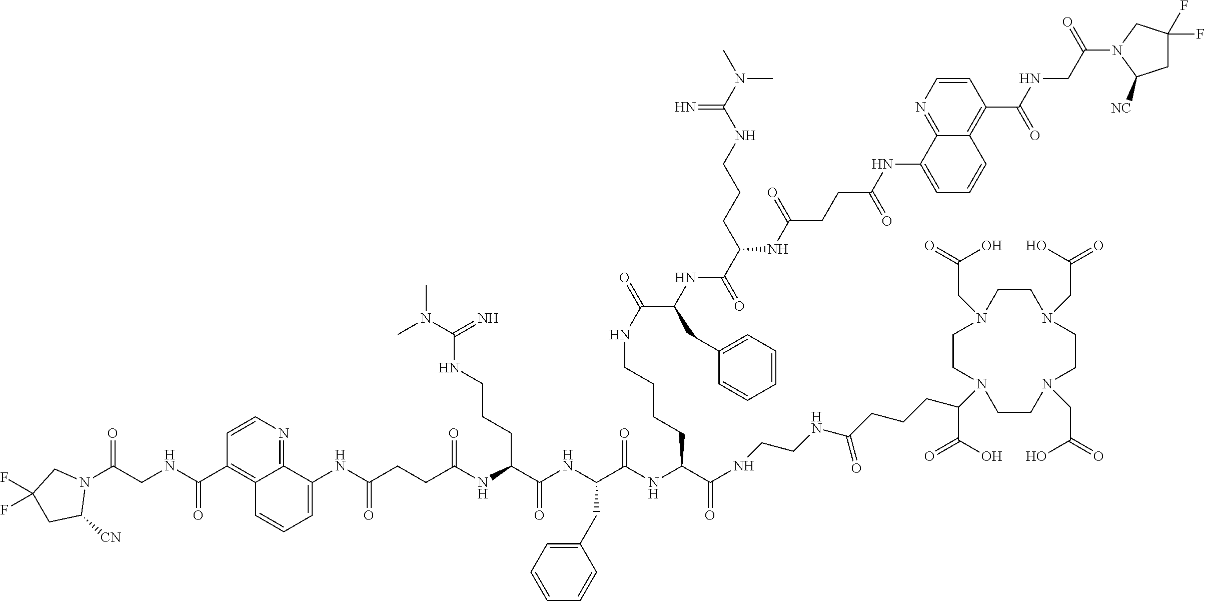 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)NCCNC(=O)CCCC(C(=O)O)N1CCN(CC(=O)O)CCN(CC(=O)O)CCN(CC(=O)O)CC1] |
| C73 | 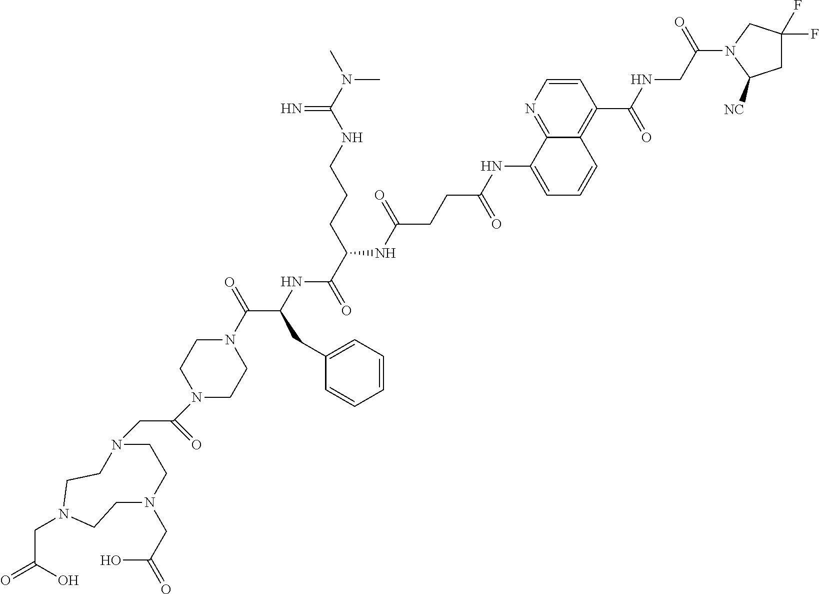 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)N1CCN(C(=O)CN2CCN(CC(=O)O)CCN(CC(=O)O)CC2)CC1] |
| C74 | 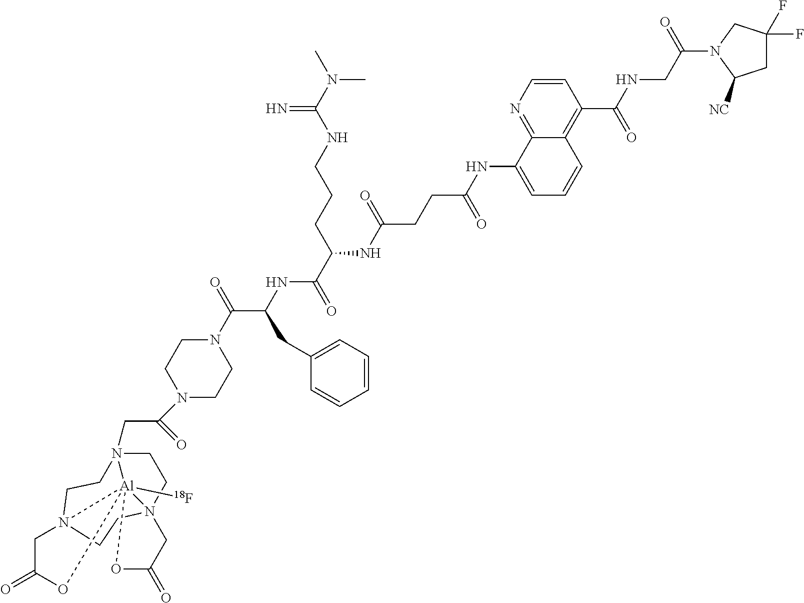 |
| C75 | 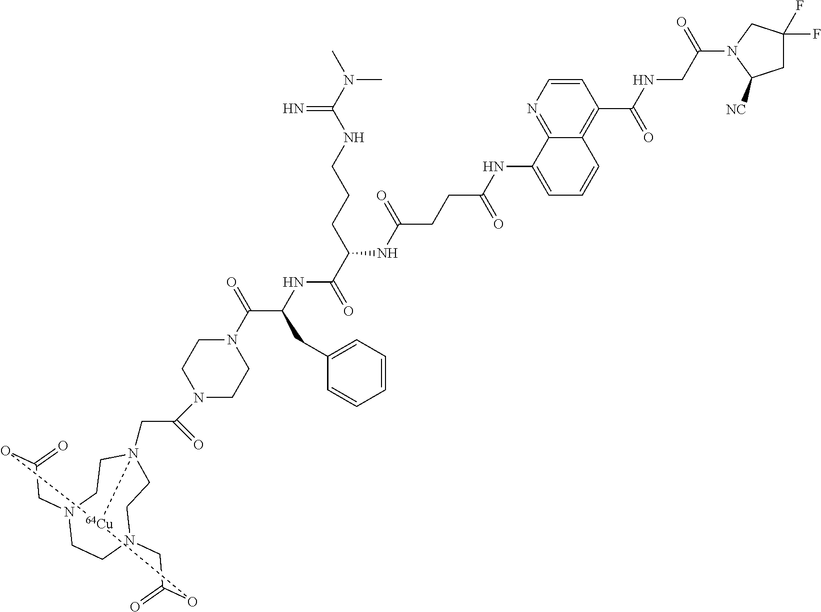 |
| C76 | 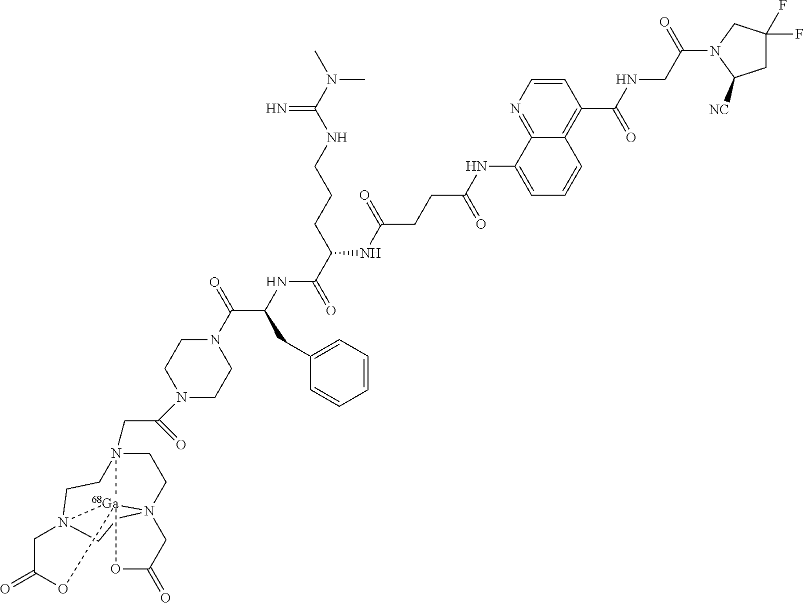 |
| C77 | 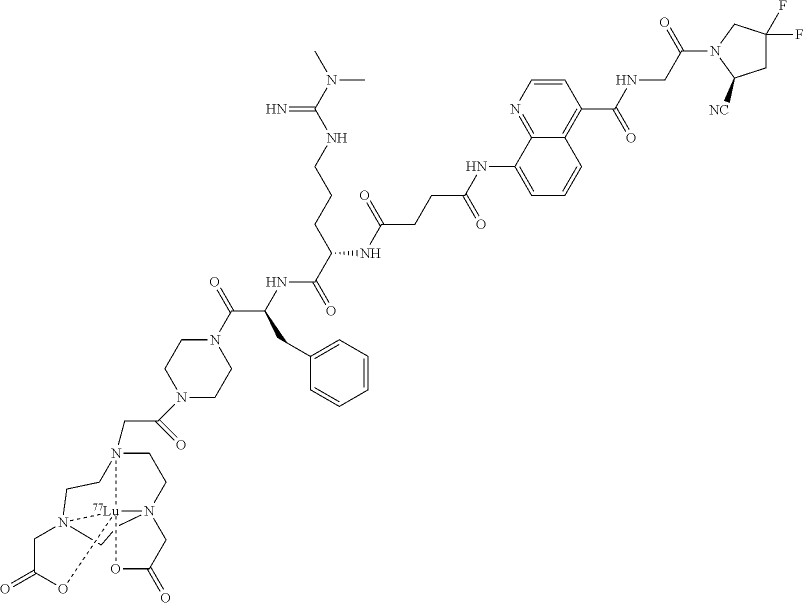 |
| C78 | 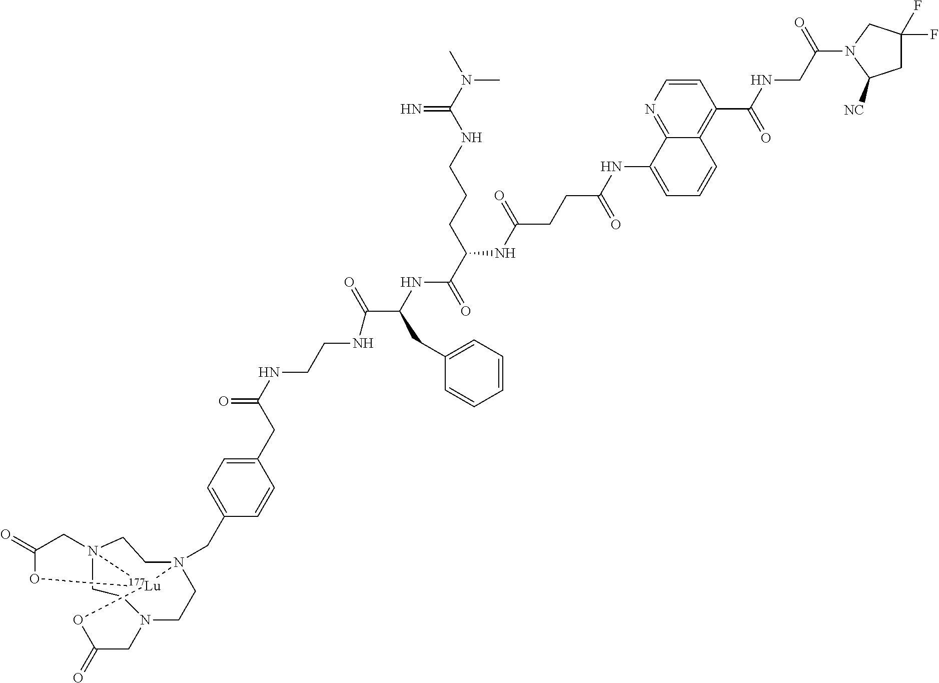 |
| C79 | 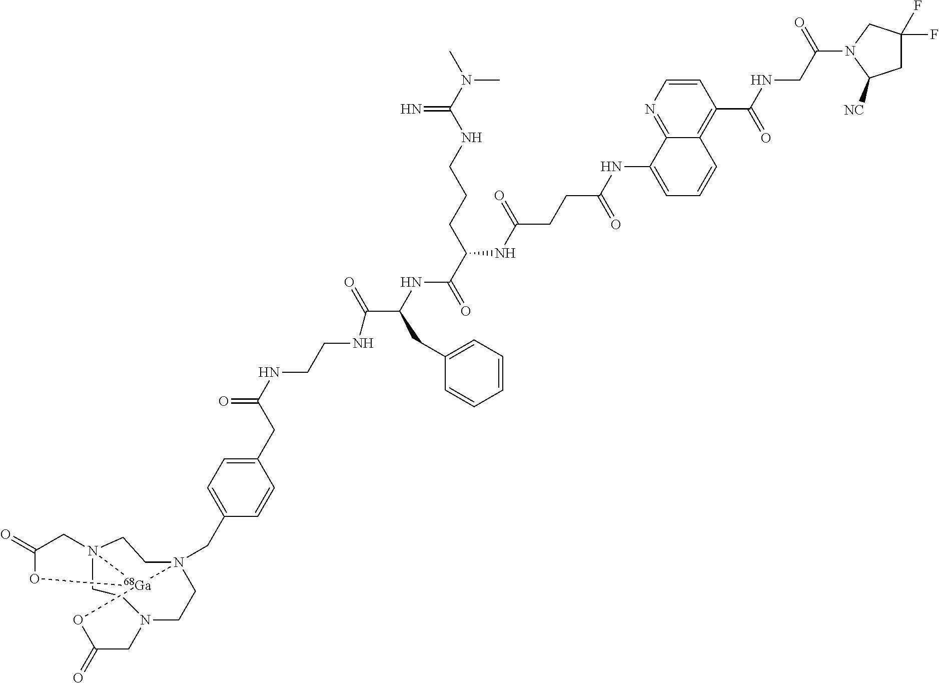 |
| C80 | 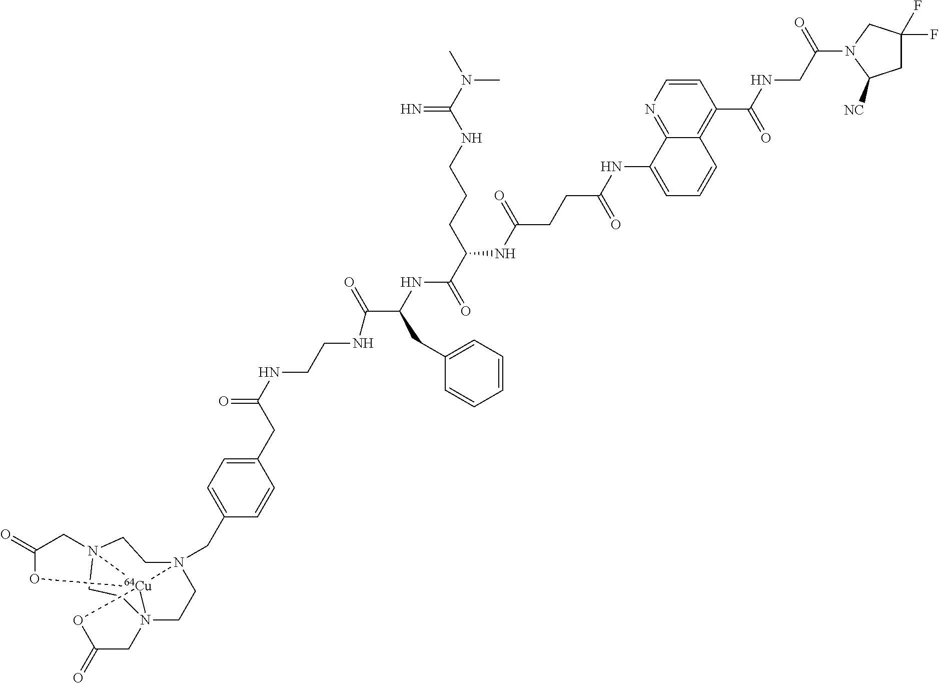 |
| C81 | 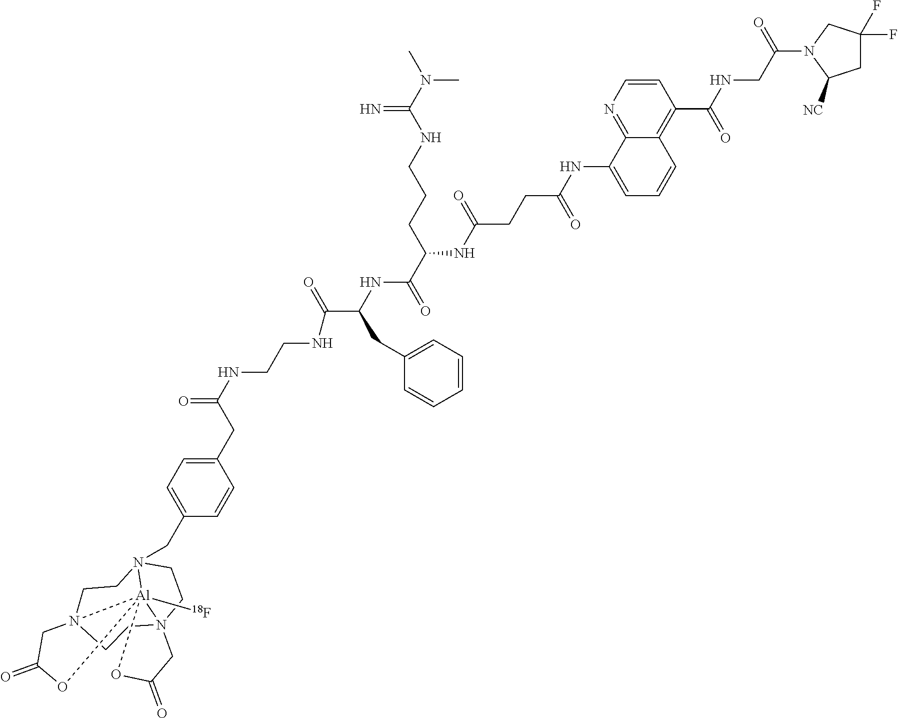 |
| C82 | 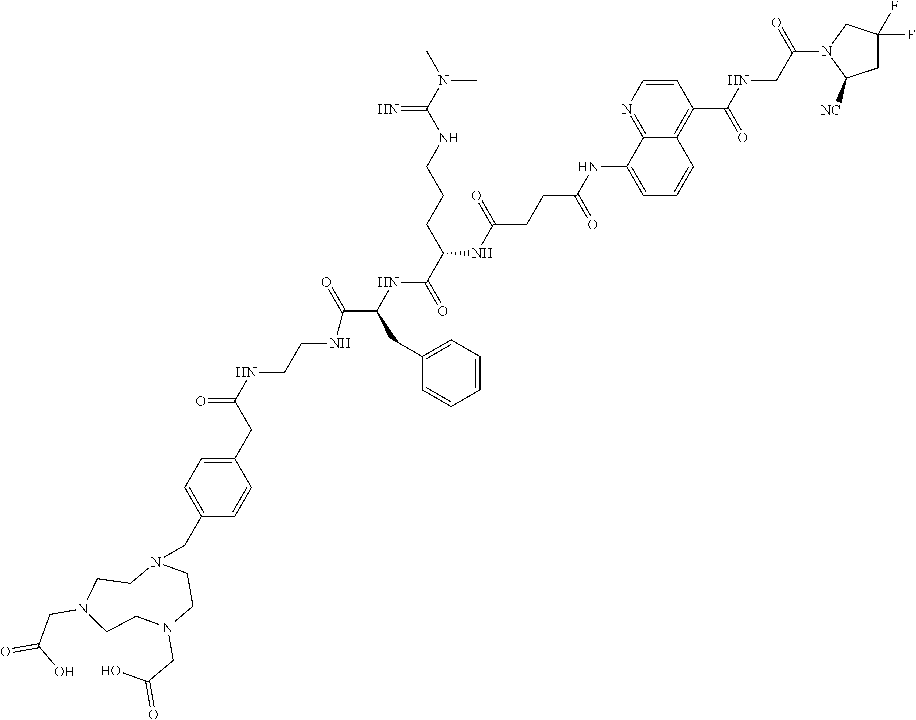 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCNC(=O)Cc1ccc(CN2CCN(CC(=O)O)CCN(CC(=O)O)CC2)cc1] |
| C83 | 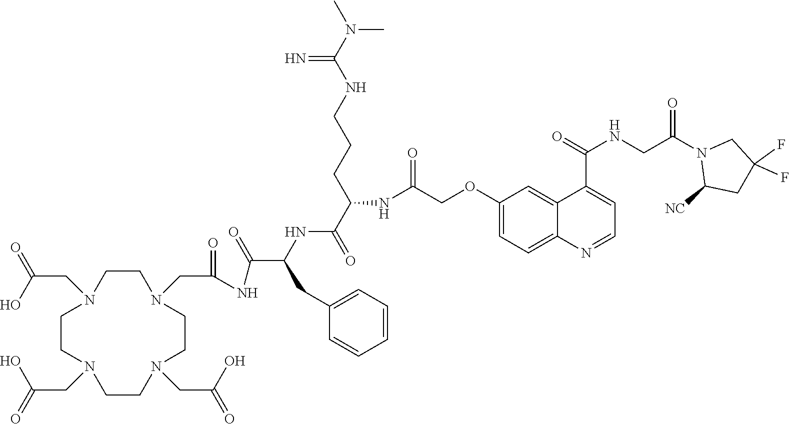 [SMILES: CN(C)C(=N)NCCC[C@H](NC(=O)COc1ccc2nccc(C(=O)NCC(=O)N3CC(F)(F)C[C@H]3C#N)c2c1)C(=O)N[C@@H](Cc1ccccc1)C(=O)NC(=O)CN1CCN(CC(=O)O)CCN(CC(=O)O)CCN(CC(=O)O)CC1] |
| C84 | 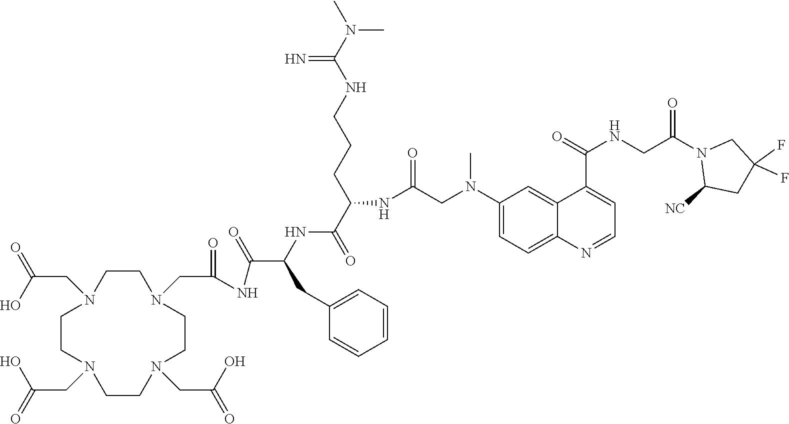 [SMILES: CN(C)C(=N)NCCC[C@H](NC(=O)CN(C)c1ccc2nccc(C(=O)NCC(=O)N3CC(F)(F)C[C@H]3C#N)c2c1)C(=O)N[C@@H](Cc1ccccc1)C(=O)NC(=O)CN1CCN(CC(=O)O)CCN(CC(=O)O)CCN(CC(=O)O)CC1] |
| C85 | 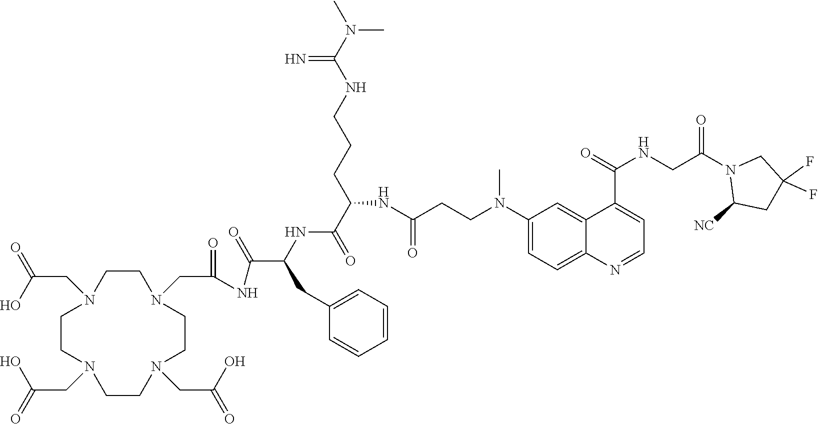 [SMILES: CN(C)C(=N)NCCC[C@H](NC(=O)CCN(C)c1ccc2nccc(C(=O)NCC(=O)N3CC(F)(F)C[C@H]3C#N)c2c1)C(=O)N[C@@H](Cc1ccccc1)C(=O)NC(=O)CN1CCN(CC(=O)O)CCN(CC(=O)O)CCN(CC(=O)O)CC1] |
| C86 | 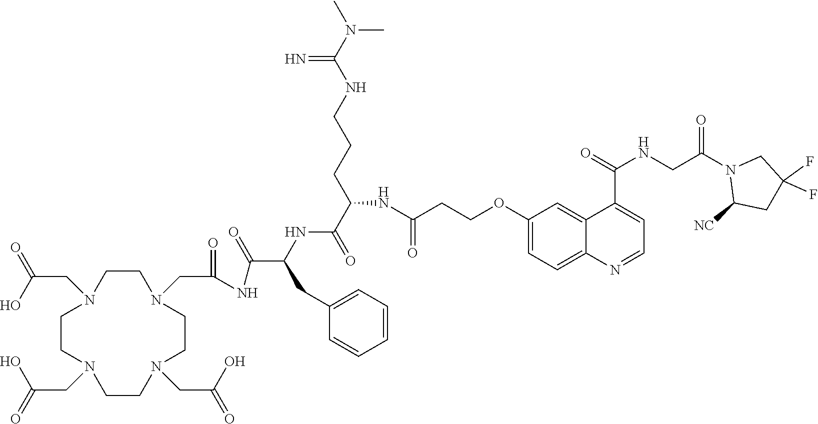 [SMILES: CN(C)C(=N)NCCC[C@H](NC(=O)CCOc1ccc2nccc(C(=O)NCC(=O)N3CC(F)(F)C[C@H]3C#N)c2c1)C(=O)N[C@@H](Cc1ccccc1)C(=O)NC(=O)CN1CCN(CC(=O)O)CCN(CC(=O)O)CCN(CC(=O)O)CC1] |
| C87 | 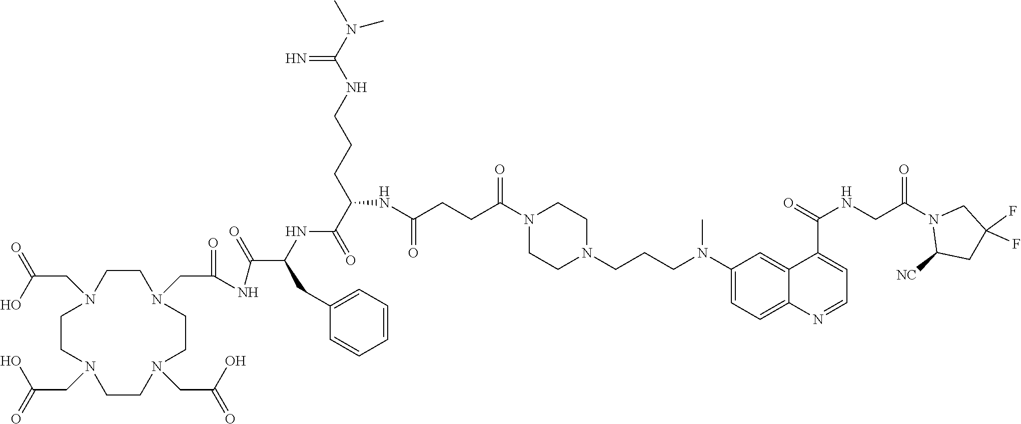 [SMILES: CN(C)C(=N)NCCC[C@H](NC(=O)CCC(=O)N1CCN(CCCN(C)c2ccc3nccc(C(=O)NCC(=O)N4CC(F)(F)C[C@H]4C#N)c3c2)CC1)C(=O)N[C@@H](Cc1ccccc1)C(=O)NC(=O)CN1CCN(CC(=O)O)CCN(CC(=O)O)CCN(CC(=O)O)CC1] |
| C88 | 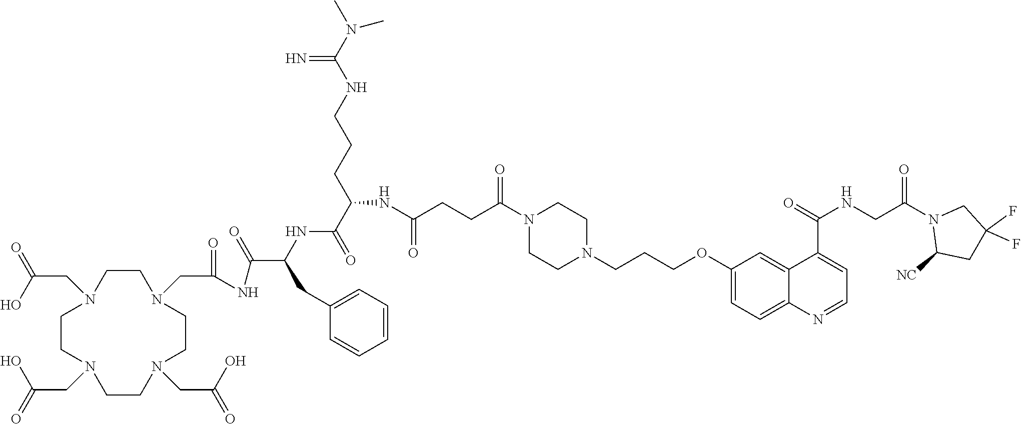 [SMILES: CN(C)C(=N)NCCC[C@H](NC(=O)CCC(=O)N1CCN(CCCOc2ccc3nccc(C(=O)NCC(=O)N4CC(F)(F)C[C@H]4C#N)c3c2)CC1)C(=O)N[C@@H](Cc1ccccc1)C(=O)NC(=O)CN1CCN(CC(=O)O)CCN(CC(=O)O)CCN(CC(=O)O)CC1] |
| C89 | 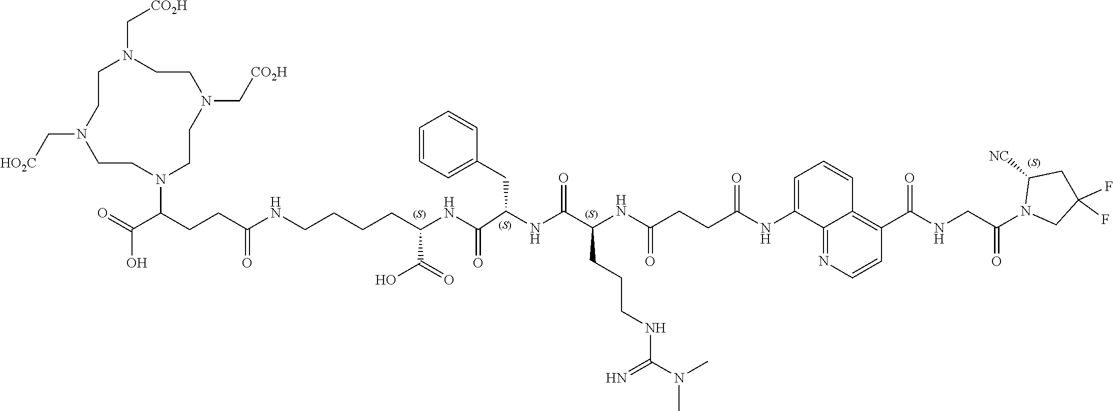 |
| C90 | 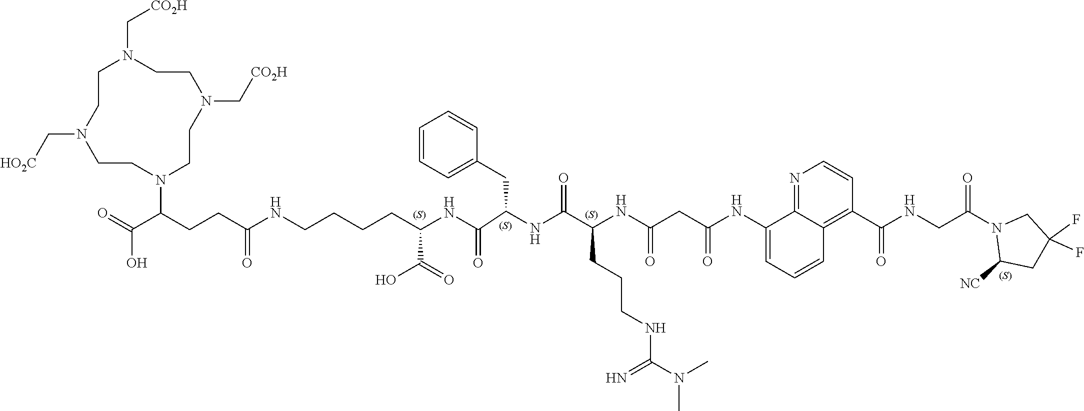 |
| C91 | 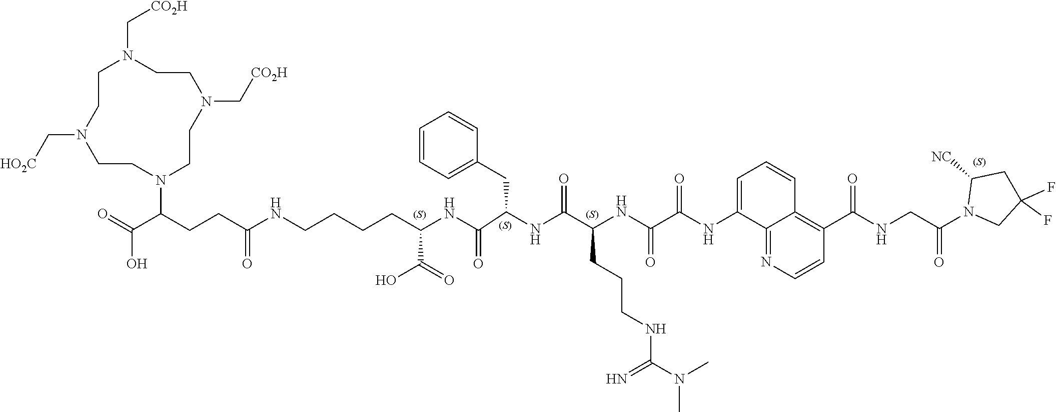 |
| C92 | 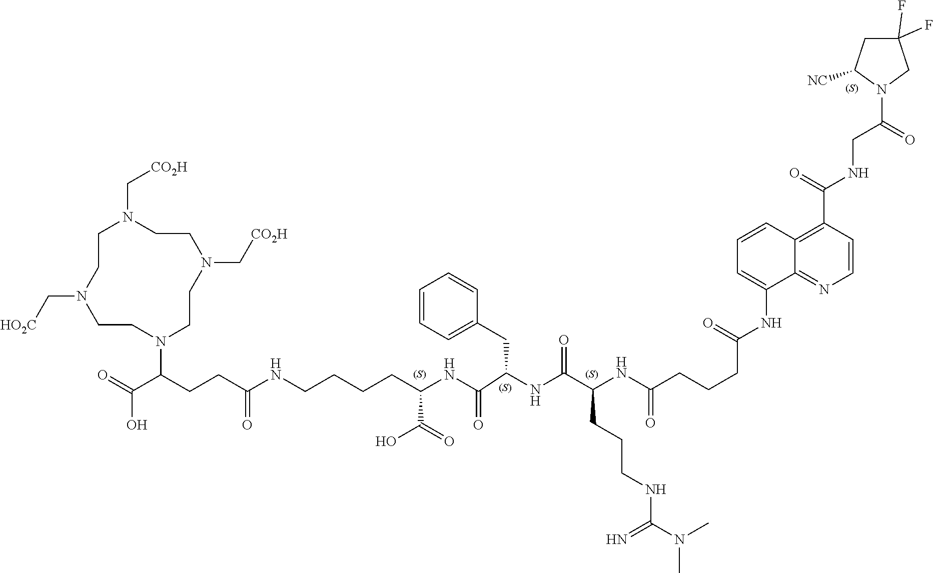 |
| C93 | 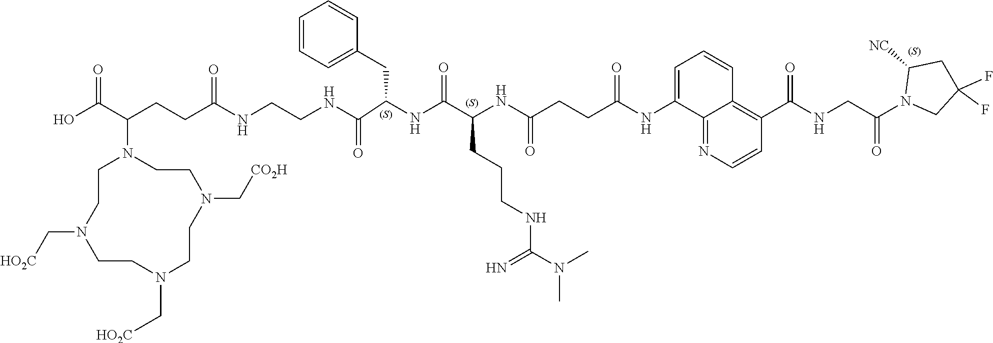 |
| C94 | 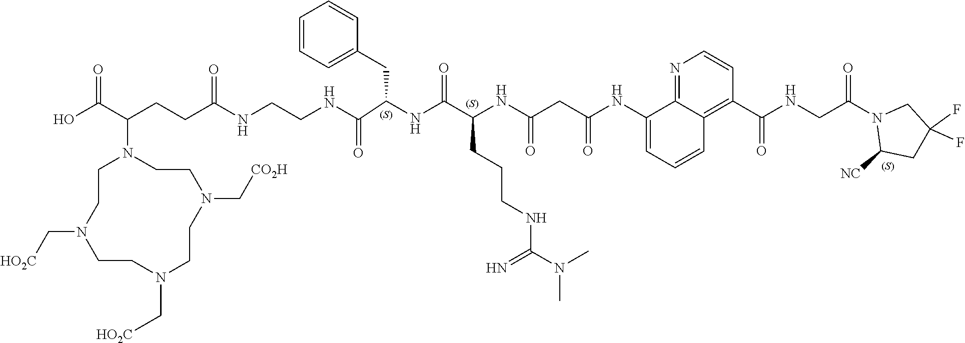 |
| C95 | 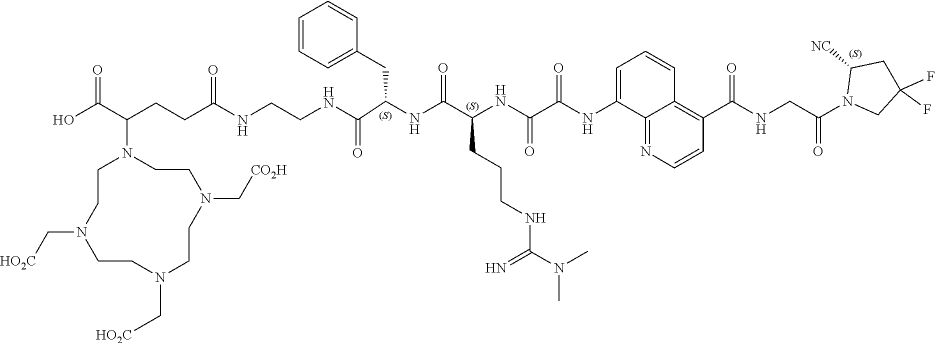 |
| C96 | 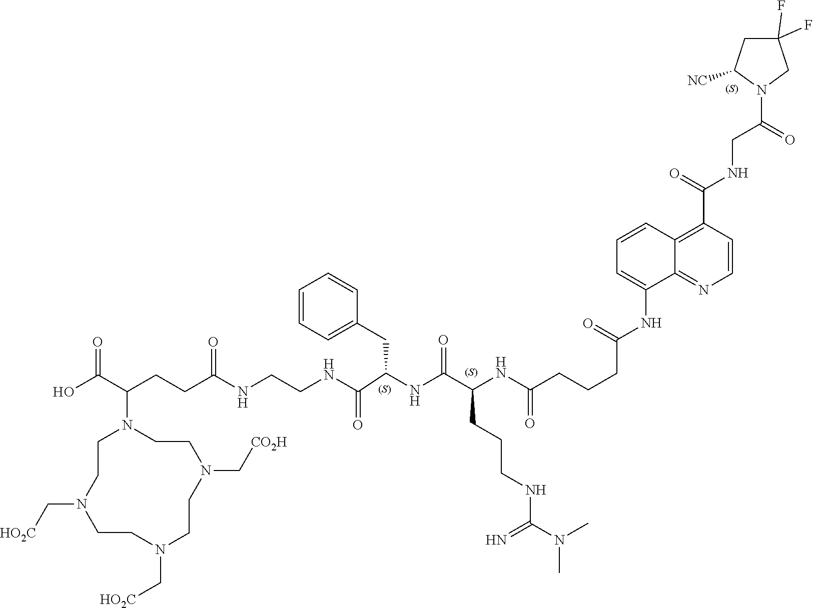 |
| C97 | 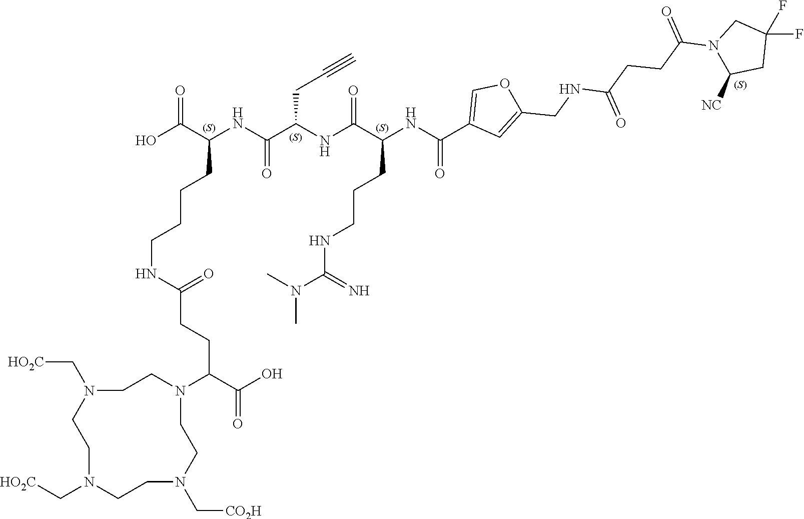 |
| C98 | 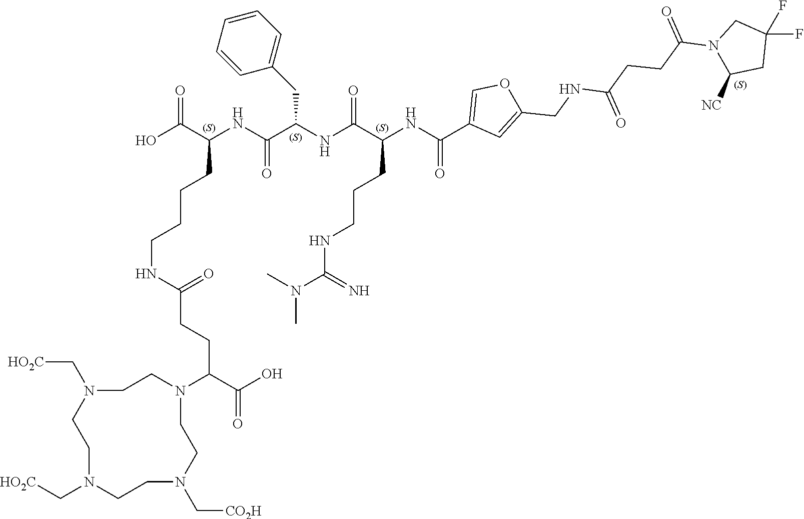 |
| C99 | 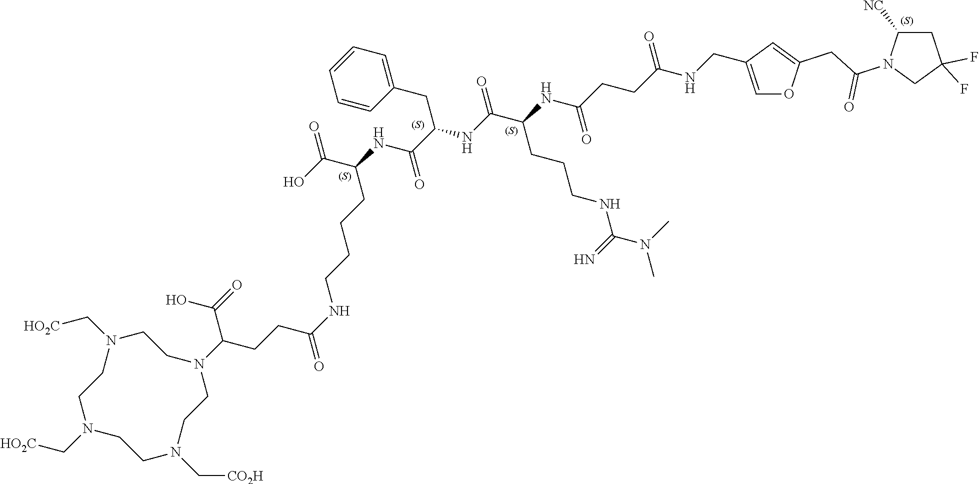 |
| C100 | 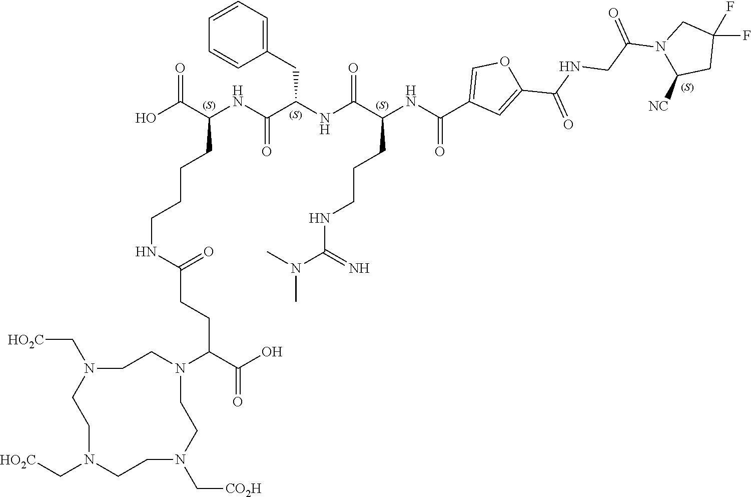 |
| C101 | 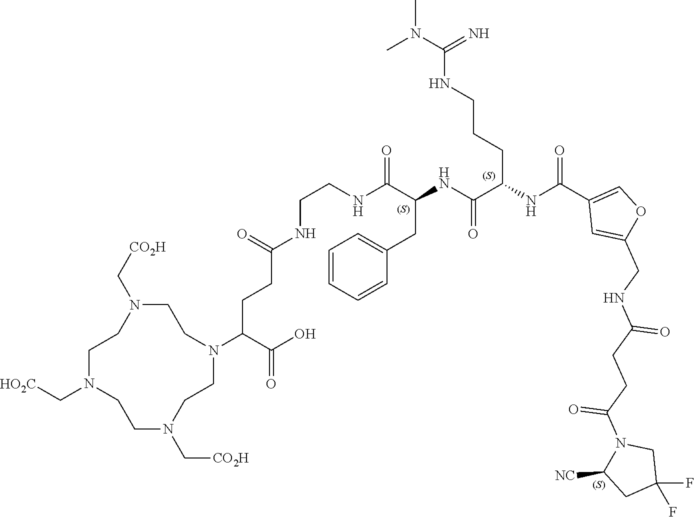 |
| C102 | 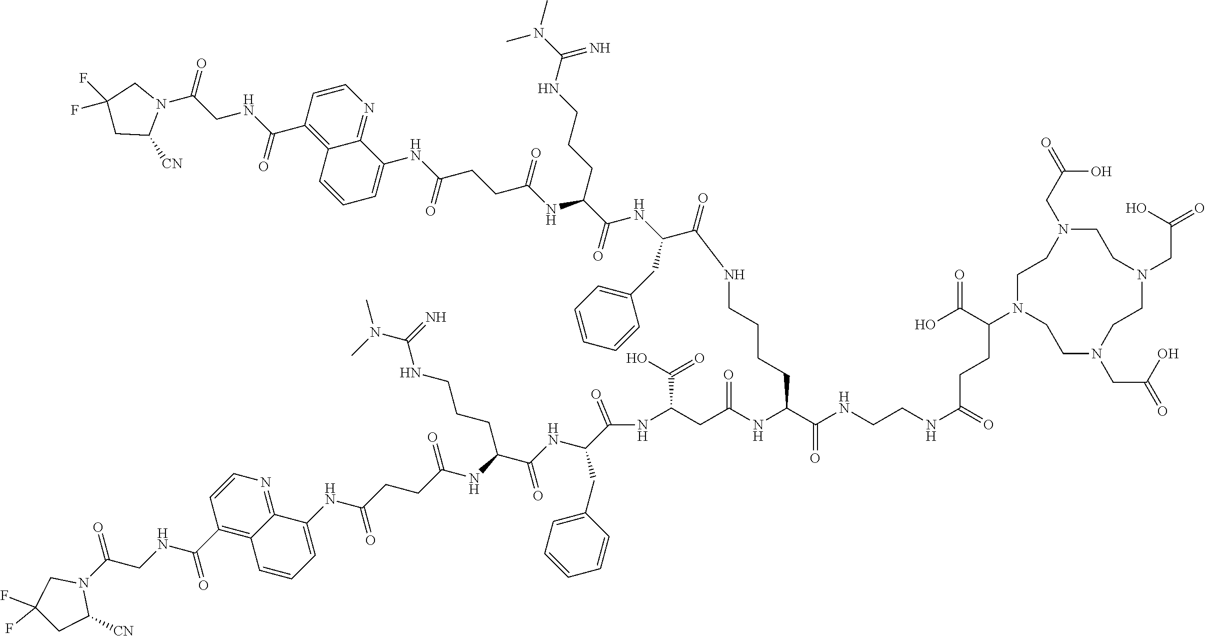 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)O)C(=O)NCCNC(=O)CCC(C(=O)O)N1CCN(CC(=O)O)CCN(CC(=O)O)CCN(CC(=O)O)CC1] |
| C103 | 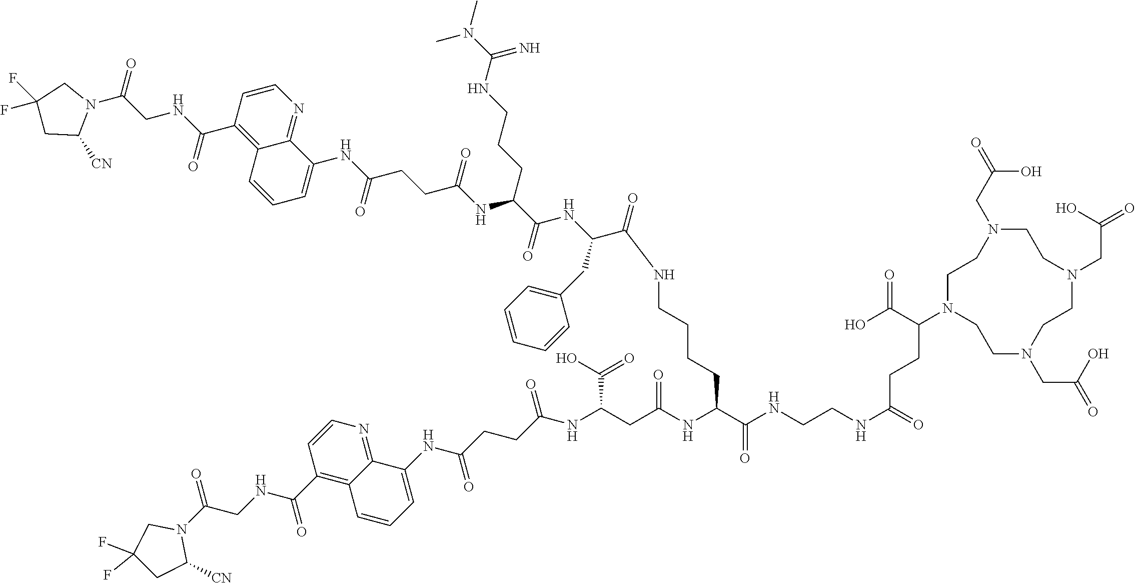 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)CCC(=O)Nc1cccc2c(C(=O)NCC(=O)N3CC(F)(F)C[C@H]3C#N)ccnc12)C(=O)O)C(=O)NCCNC(=O)CCC(C(=O)O)N1CCN(CC(=O)O)CCN(CC(=O)O)CCN(CC(=O)O)CC1] |
| C104 | 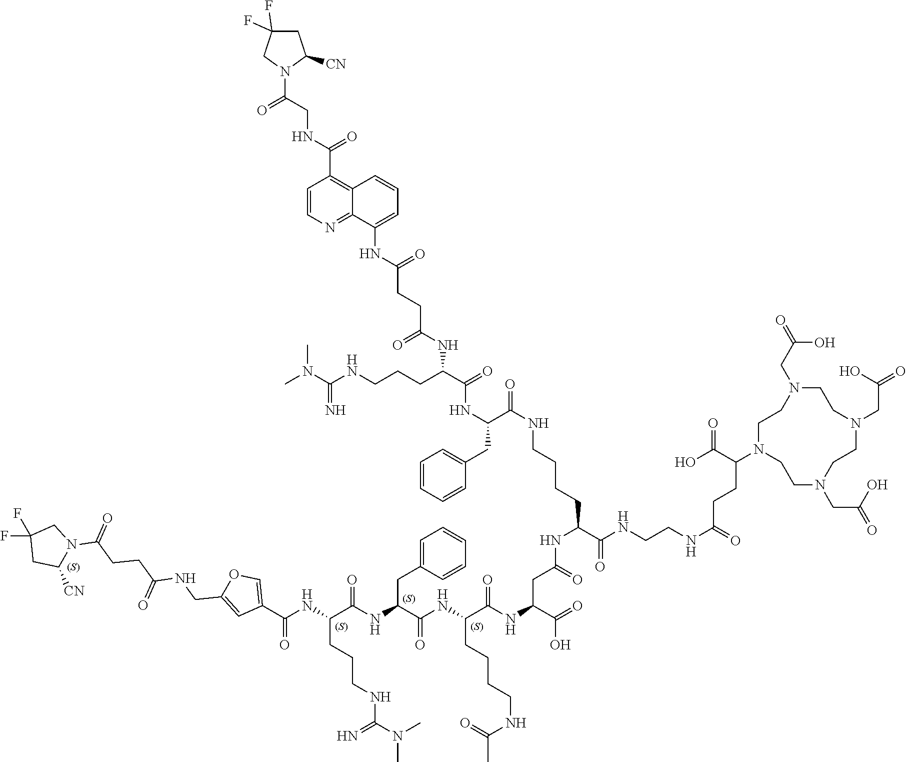 |
| C105 | 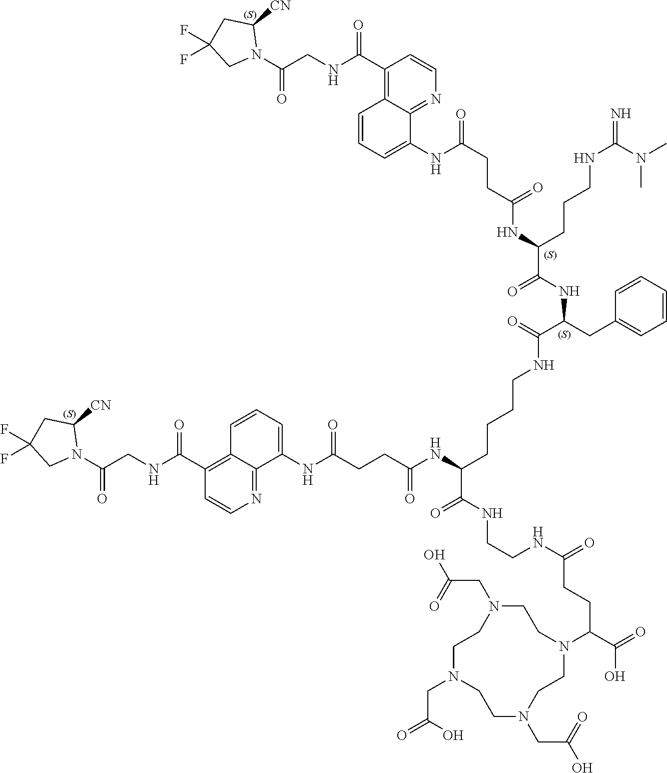 |
| C106 | 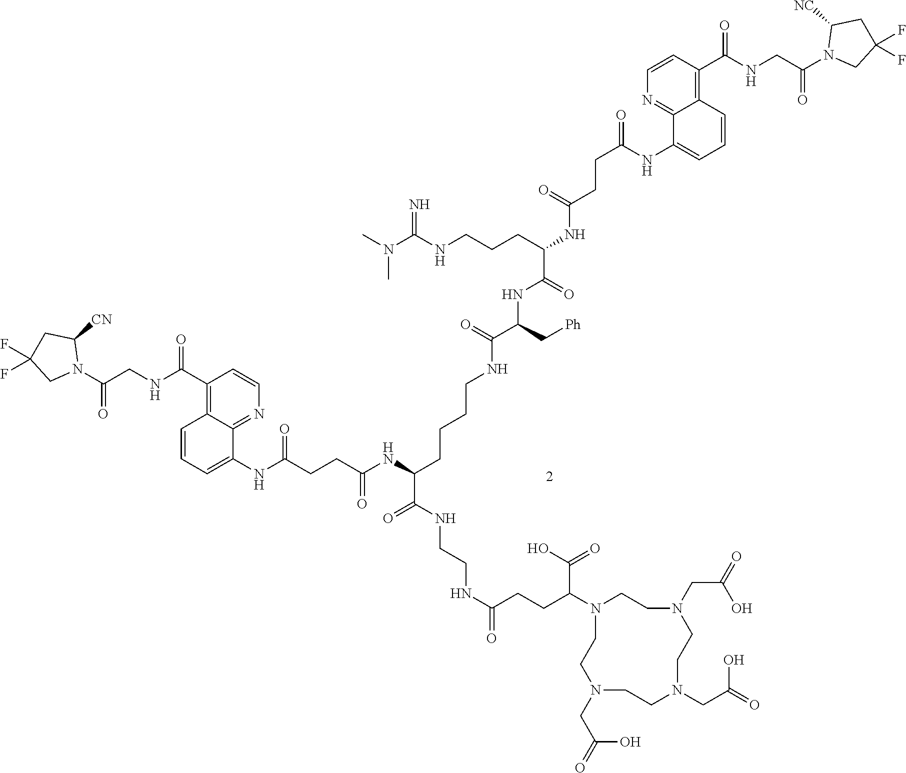 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)CCC(=O)Nc1cccc2c(C(=O)NCC(=O)N3CC(F)(F)C[C@H]3C#N)ccnc12)C(=O)NCCNC(=O)CCC(C(=O)O)N1CCN(CC(=O)O)CCN(CC(=O)O)CCN(CC(=O)O)CC1] |
| 28 | 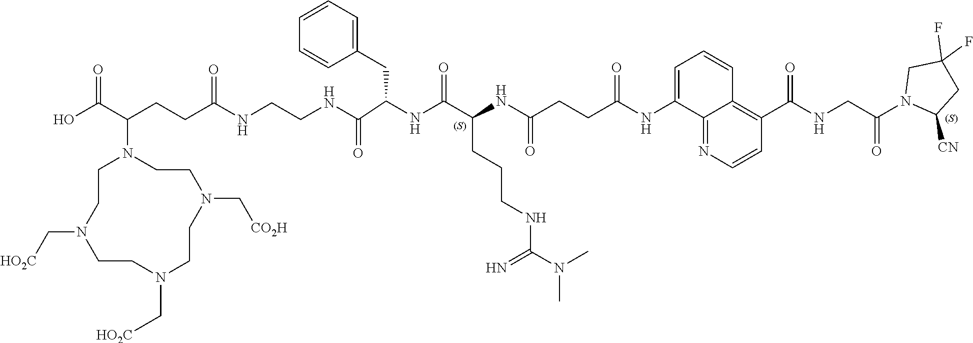 |
| 28a | 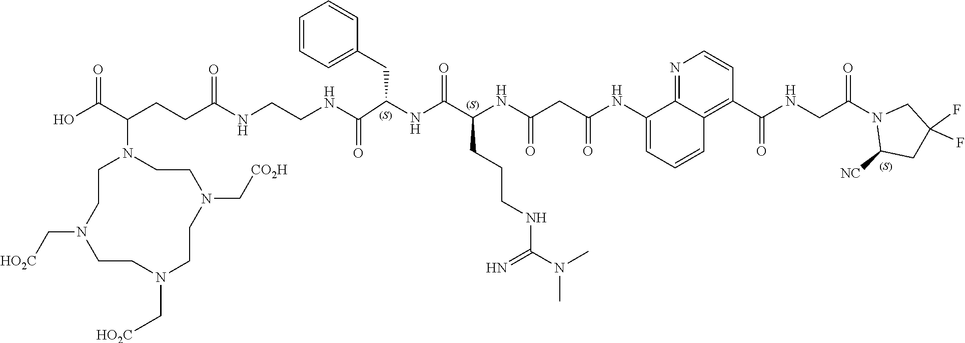 |
| 28b | 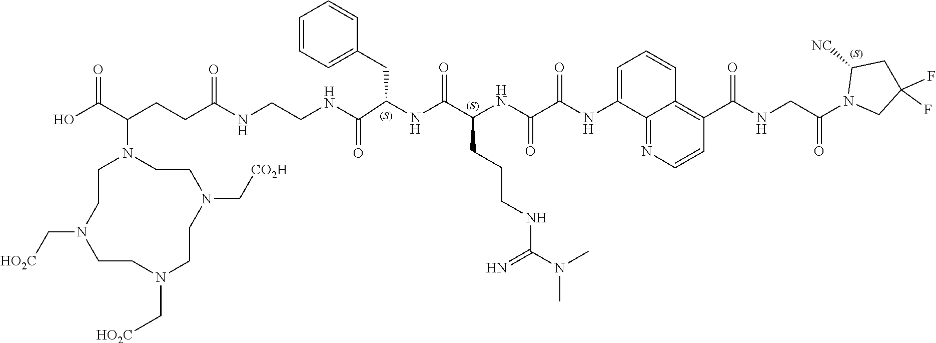 |
| 28c | 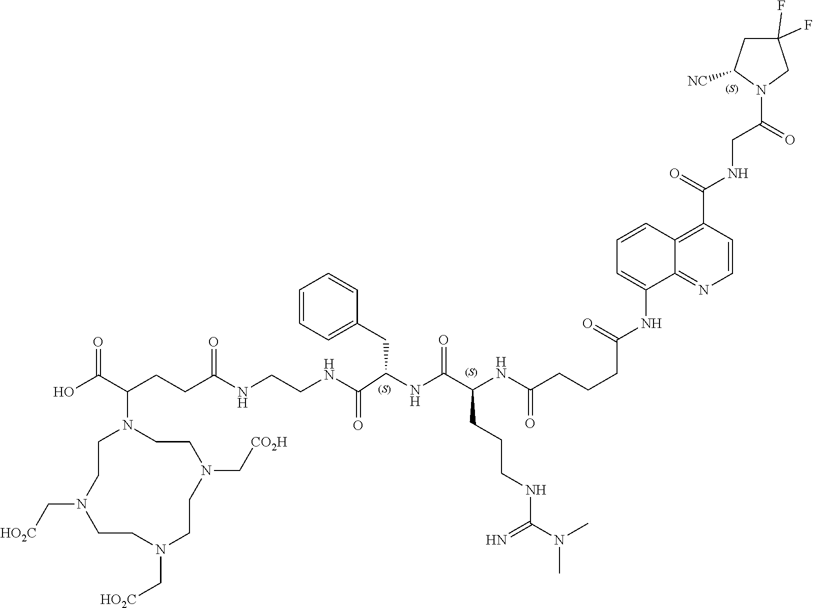 |
| 29 | 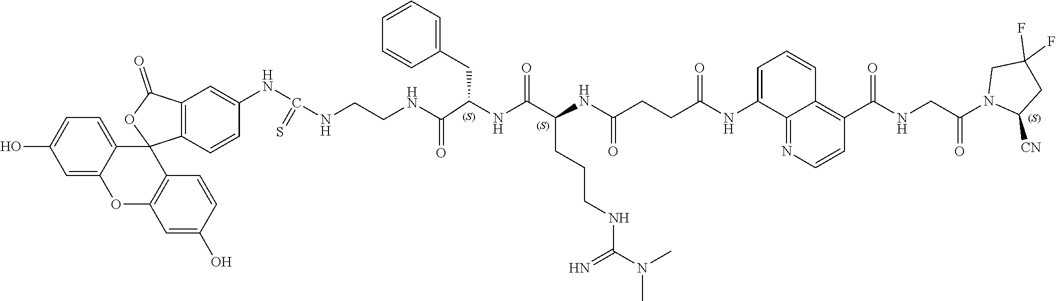 |
| 32 | 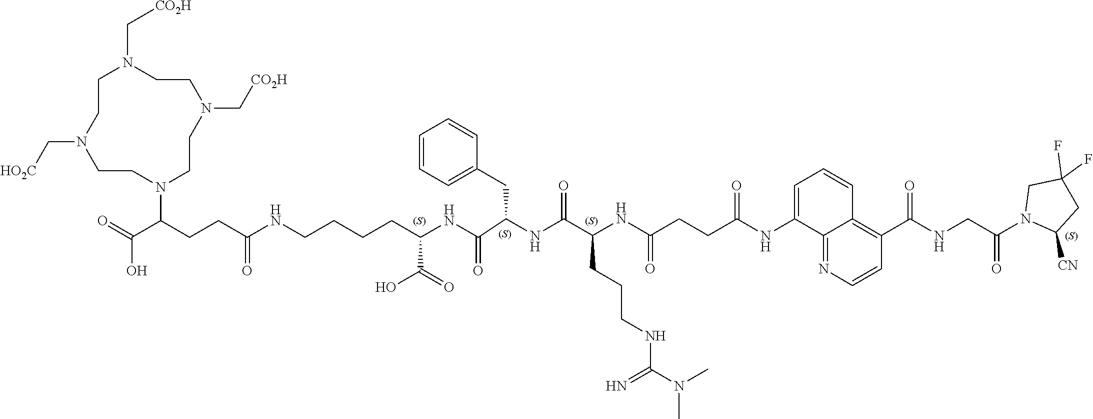 |
| 32a | 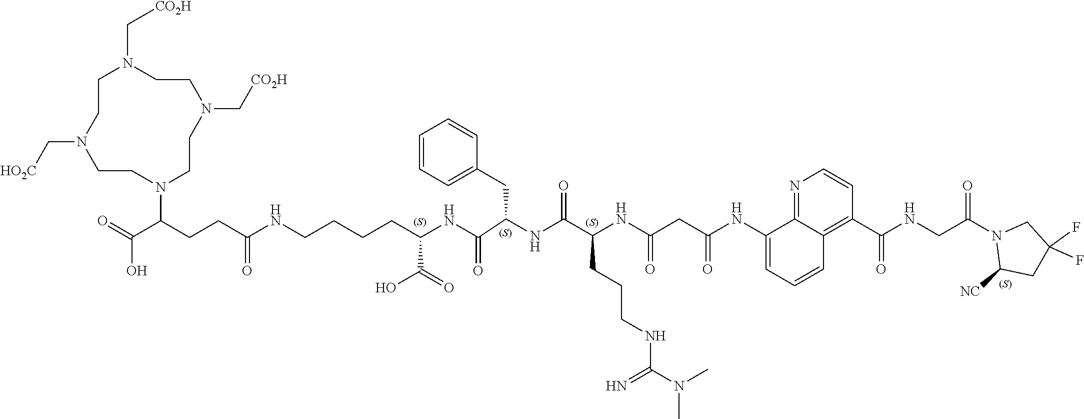 |
| 32b | 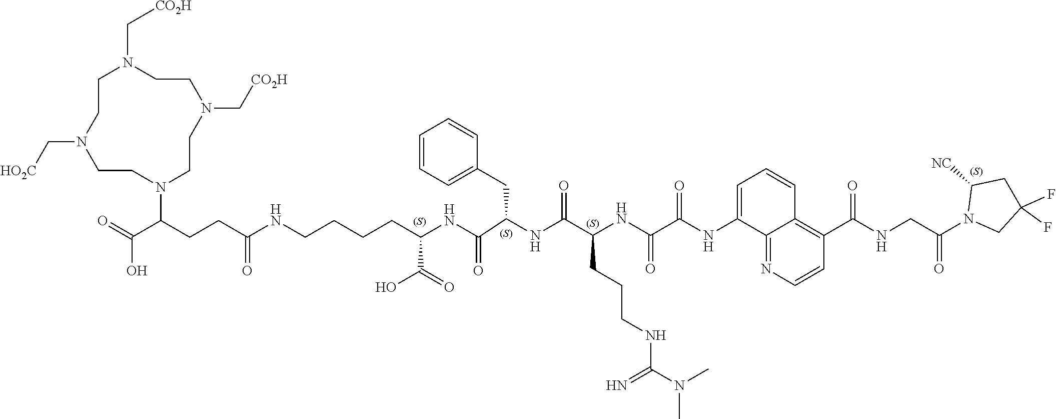 |
| 32c | 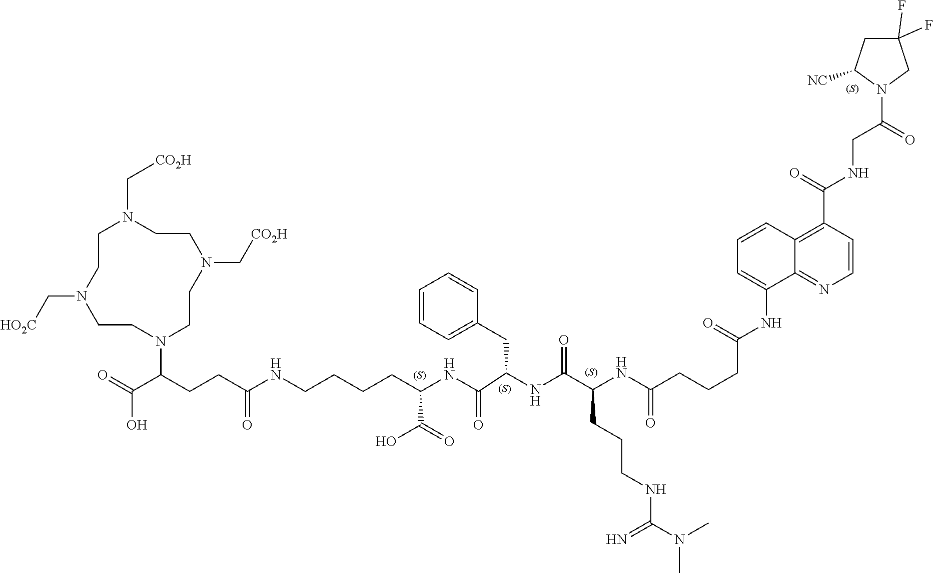 |
| 33 | 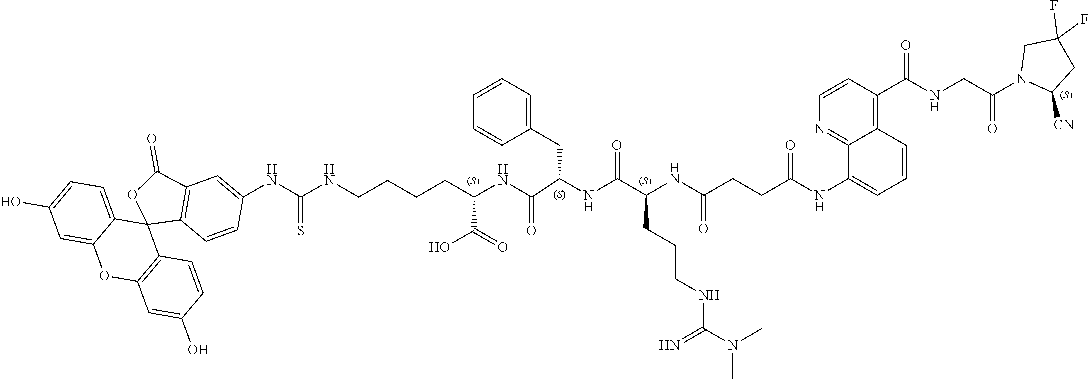 |
| C200 | 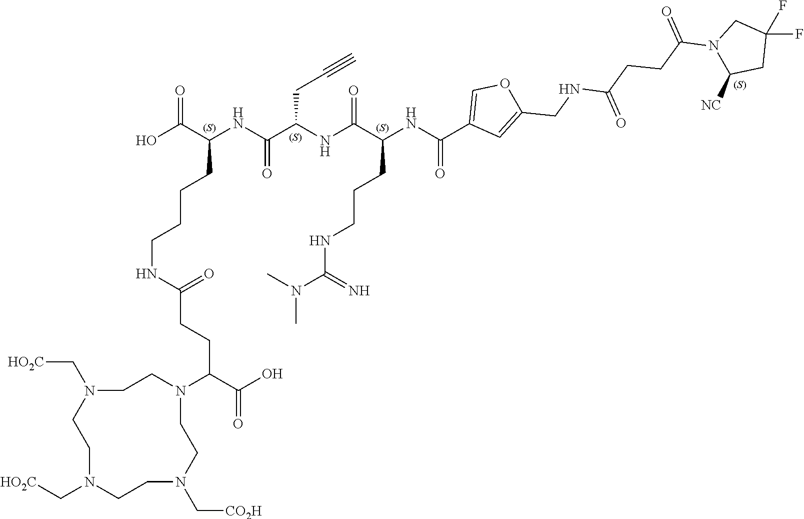 |
| C201 | 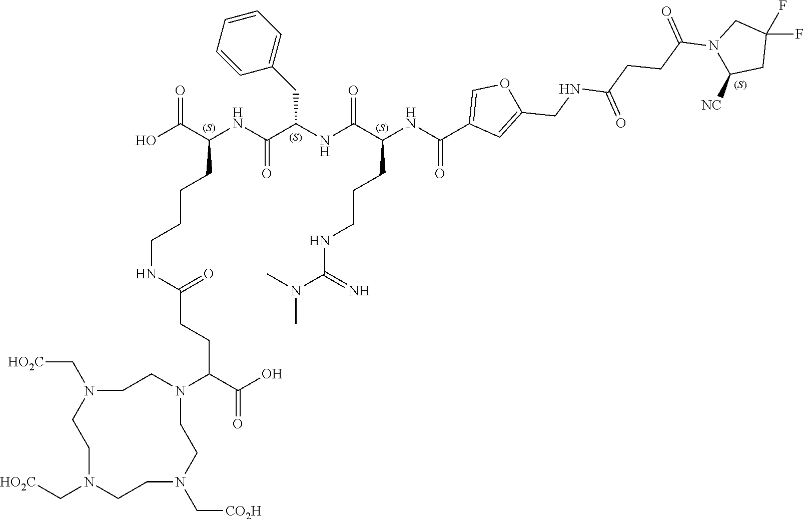 |
| C202 | 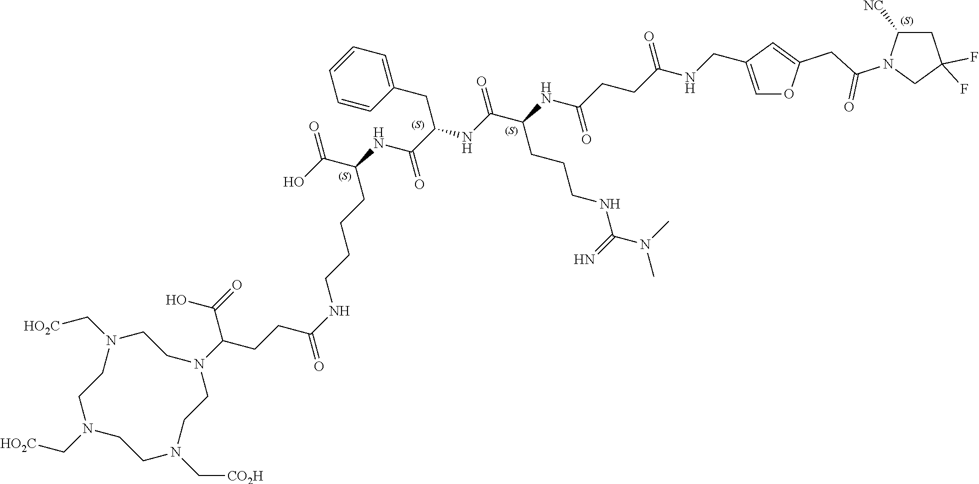 |
| C203 | 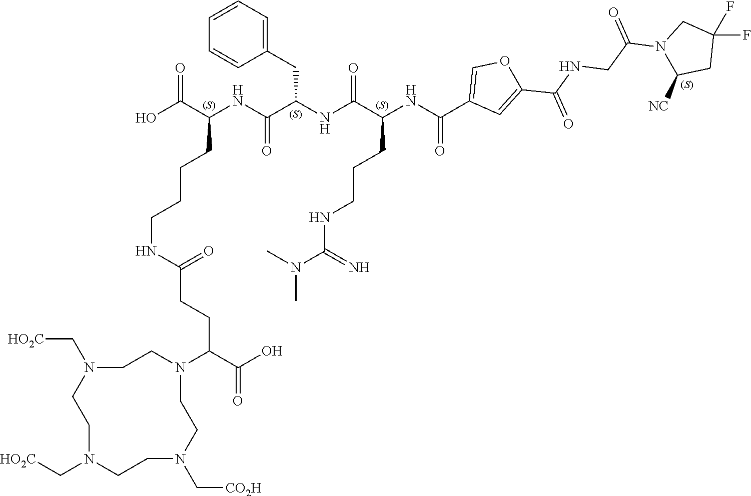 |
| C204 | 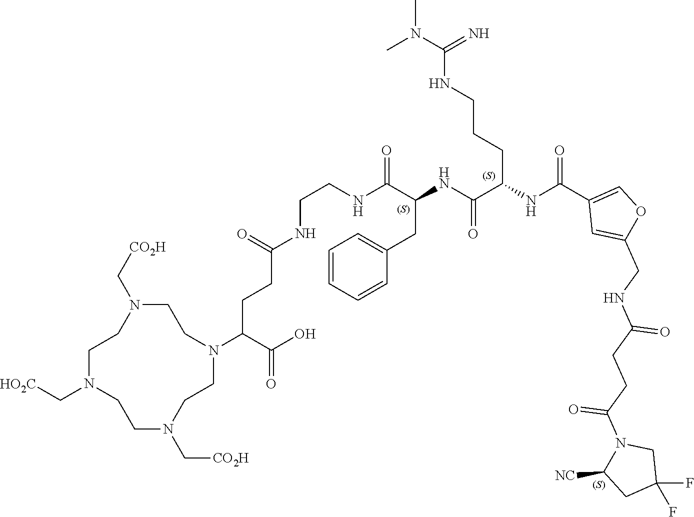 |
| C205 | 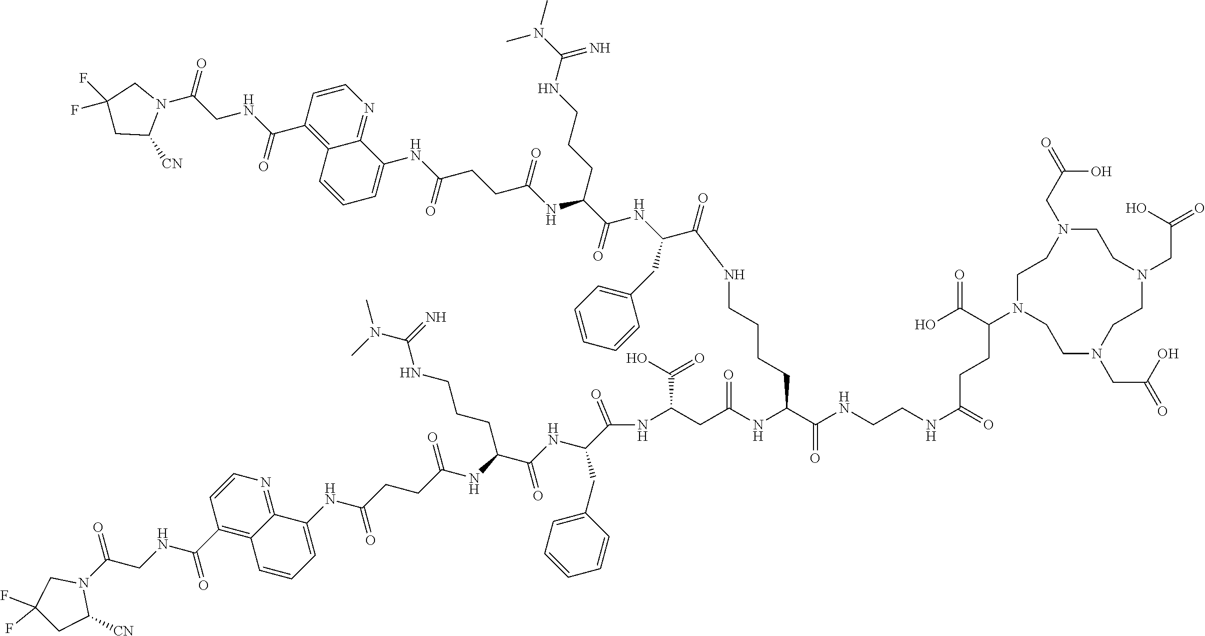 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)[C@H](Cc1ccccc1)NC(=O)[C@H](CCCNC(=N)N(C)C)NC(=O)CCC(=O)Nc1cccc2c(C(=O)NCC(=O)N3CC(F)(F)C[C@H]3C#N)ccnc12)C(=O)O)C(=O)NCCNC(=O)CCC(C(=O)O)N1CCN(CC(=O)O)CCN(CC(=O)O)CCN(CC(=O)O)CC1] |
| C206 | 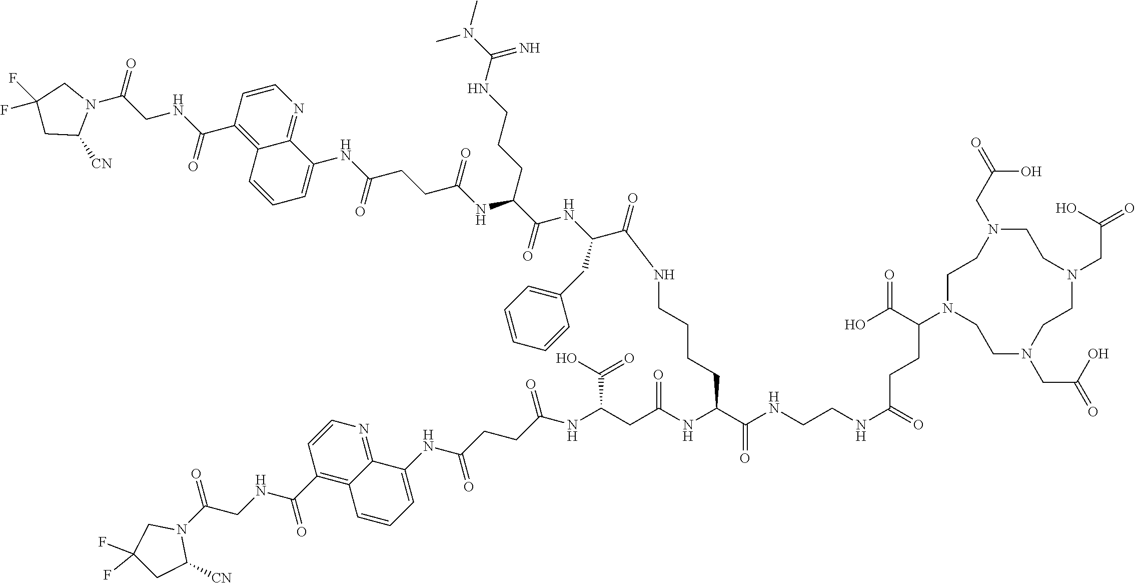 [SMILES: CN(C)C(=N)NCCC[C@H](NC(=O)CCC(=O)Nc1cccc2c(C(=O)NCC(=O)N3CC(F)(F)C[C@H]3C#N)ccnc12)C(=O)N[C@@H](Cc1ccccc1)C(=O)NCCCC[C@H](NC(=O)C[C@H](NC(=O)CCC(=O)Nc1cccc2c(C(=O)NCC(=O)N3CC(F)(F)C[C@H]3C#N)ccnc12)C(=O)O)C(=O)NCCNC(=O)CCC(C(=O)O)N1CCN(CC(=O)O)CCN(CC(=O)O)CCN(CC(=O)O)CC1] |
| C207 | 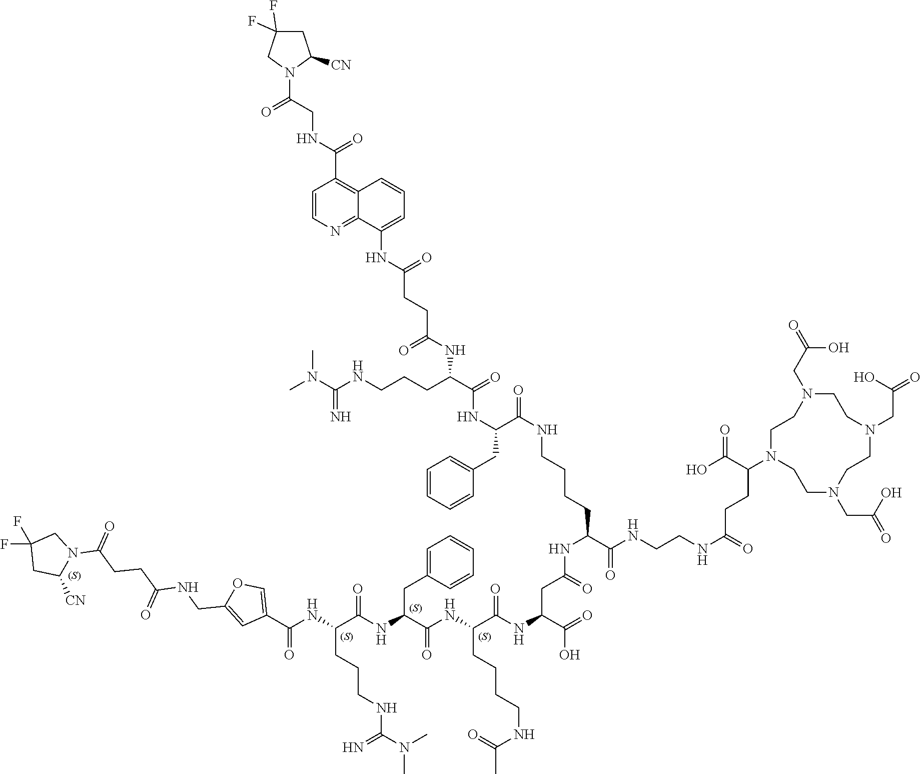 |

The present disclosure further comprises the following embodiments.

1. A compound, its individual diastereoisomers, its hydrates, its solvates, its crystal forms, its individual tautomers or a pharmaceutically acceptable salt thereof, wherein the compound structure comprises at least one moiety A independently represented by the following structure:

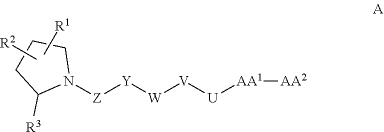

wherein AA¹ has the following structure:

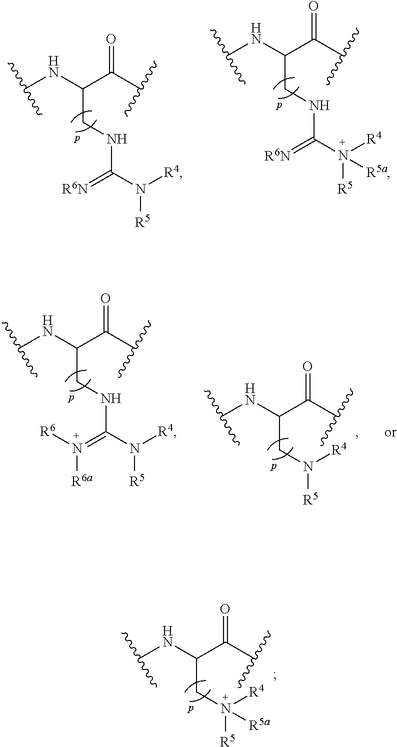

each of R¹ and R² is independently selected from the group consisting of H, OH, halogen, C₁₋₆alkyl, —O—C₁₋₆alkyl, and —S—C₁₋₆alkyl, N₃, preferably H or halogen, more preferably F;
R³ is independently selected from H, —CN, —B(OH)₂, —C(O)alkyl, —C(O)haloalkyl, —C(O)aryl-, —C═C—C(O)aryl, —C═C—S(O)₂aryl, —CO₂H, —SO₃H, —SO₂NH₂, —PO₃H₂, and 5-tetrazolyl, preferably —CN, —B(OH)₂, or —CO₂H, more preferably —CN;
each of R⁴, R⁵, R^{5a}, R⁶, and R^{6a} is independently H, OH, optionally substituted C₁-C₆ alkyl, optionally substituted C₁-C₆ haloalkyl, optionally substituted —C(O)C₁-C₅ alkyl, or optionally substituted —C(O)C₁-C₅ haloalkyl, and wherein two of them can be joined together or any one of them can be joined to the (CH₂)ₚ moiety to form a ring structure, preferably wherein at least one of R⁴, R⁵, R^{5a}, R⁶, and R^{6a} is not H;
p is 1 to 5, preferably 2, 3 or 4, more preferably 3;
AA² is absent, or represents

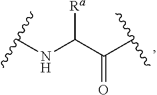

wherein R^{a} is selected from H, optionally substituted C₁₋₆ alkyl, (C₃-C₁₀ carbocyclyl)C₁₋₆ alkyl, (C₆-C₁₀ aryl)C₁₋₆ alkyl, (C₁-C₁₀ heterocyclyl)C₁₋₆ alkyl, C₂₋₆ alkenyl, C₂₋₆ alkynyl, and C₆-C₁₀ aryl, in each of which optionally one or more of the carbon atoms can be replaced by heteroatoms; preferably, R^{a} is a side chain derived from a proteinogenic or non-proteinogenic amino acid; more preferably, R^{a} is H, optionally substituted C₁₋₆ alkyl, (C₆-C₁₀ aryl)C₁₋₄ alkyl, or (C₂₋₆ alkynyl)C₁₋₄ alkyl;
Z is C(O), 0, CH₂, NR′, C═O, C═S, C=NR′, HCR′ and R′CR′, with the proviso that two Os are not directly adjacent to each other, and is preferably C(O);
Y is a single bond or a linking group, preferably an optionally substituted C₁₋₁₆ aliphatic group, in which optionally one or more carbon atoms can be replaced by heteroatoms, and which can be saturated optionally contain one or more double or triple bonds;
W is a carbocyclic or heterocyclic group;
V is a single bond or a linking group, preferably an optionally substituted C₁₋₁₆ aliphatic group, in which optionally one or more carbon atoms can be replaced by heteroatoms, and which can be saturated optionally contain one or more double or triple bonds;
U is single bond or C(O), 0, CH₂, NR′, C═O, C═S, C=NR′, HCR′ and R′CR′, preferably C(O); and
each R′ is independently H or selected from H, SH, NH₂, halogen, cyano, carboxy, C₁₋₆-alkyl, O(C₁₋₆ alkyl), S(C₁₋₆-alkyl), C₂₋₆ alkenyl, C₂₋₆ alkynyl, C₁₋₆ heteroalkenyl, C₁₋₆ heteroalkynyl, C₃₋₁₀ cycloalkenyl, C₁₋₁₀ cycloheteroalkenyl, C₆₋₁₀ aryl, and (C₆₋₁₀ aryl)C₁₋₆ alkyl, each of which being optionally substituted with from 1 to 3 substituents selected from —OH, oxo and halo, and optionally connected to an atom in Z, Y, W or V.

2. The compound of embodiment 1, wherein the group

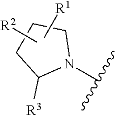

has the structure

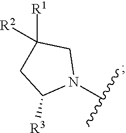

preferably:

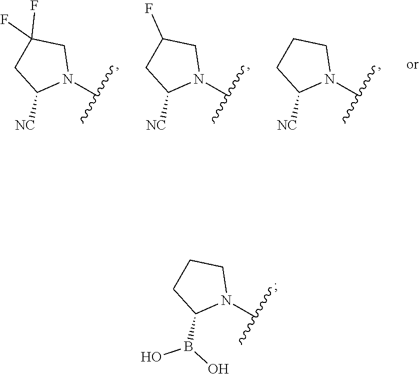 [SMILES: *N1CC(F)(F)C[C@H]1C#N ; *N1CC(F)C[C@H]1C#N ; *N1CCC[C@H]1C#N ; *N1CCC[C@H]1B(O)O]

or OH more preferably

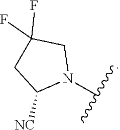 [SMILES: *N1CC(F)(F)C[C@H]1C#N]

3. The compound of any one of the preceding embodiments, wherein AA¹ has the structure:

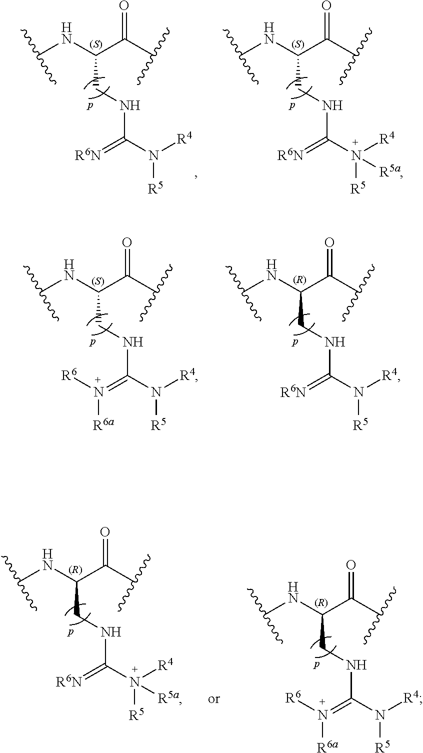

preferably:

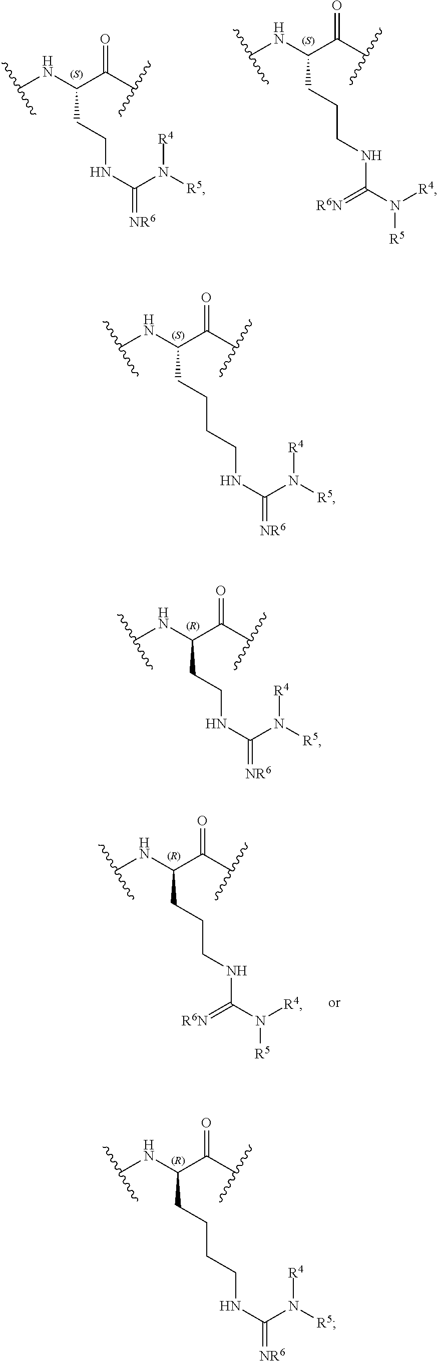

more preferably:

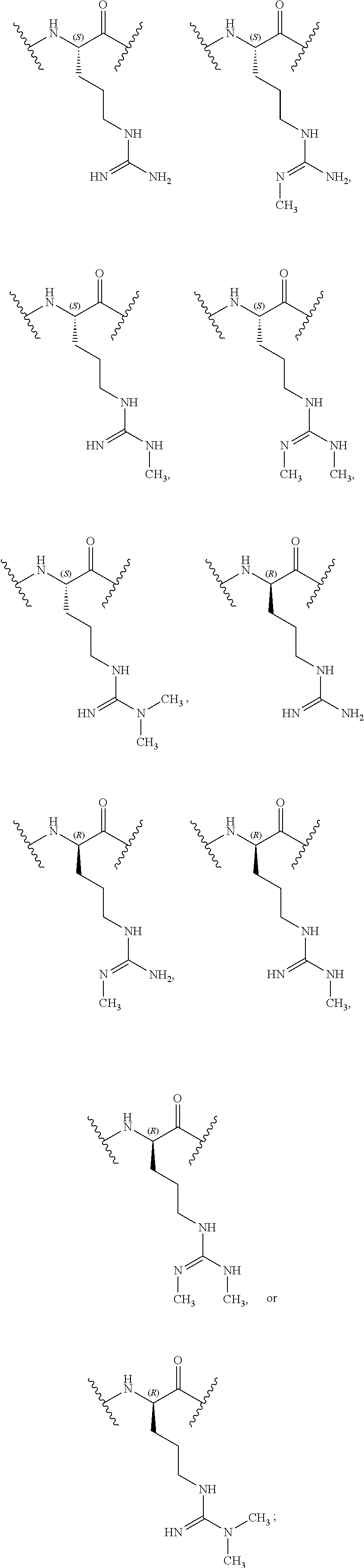

most preferably:

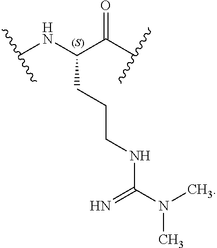

4. The compound of any one of the preceding embodiments, wherein A or R³

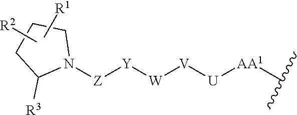

is represented by one if the following structures A¹ to A⁶:

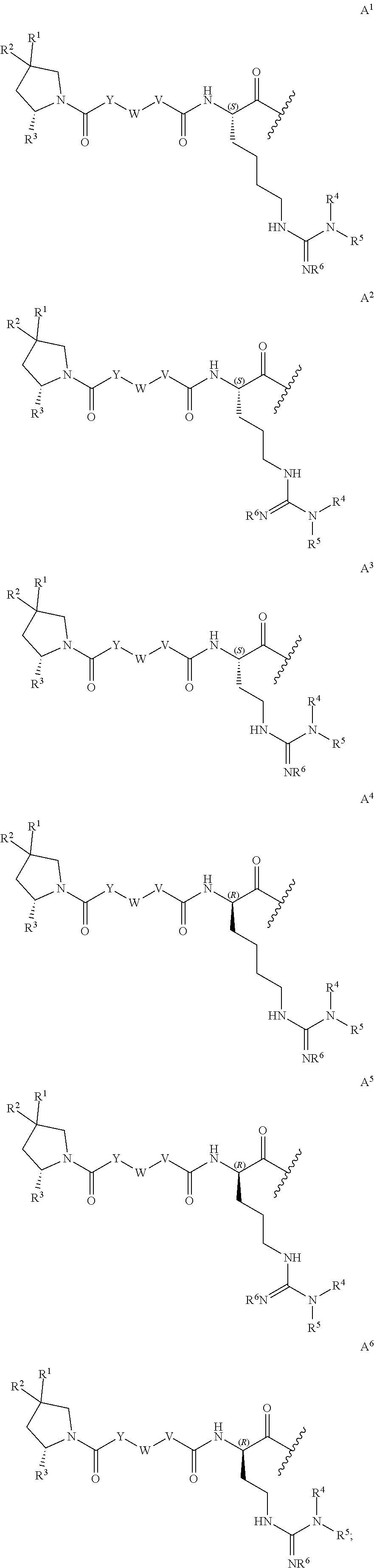

preferably A^{2′} or A^{5′}, more preferably A^{2′}:

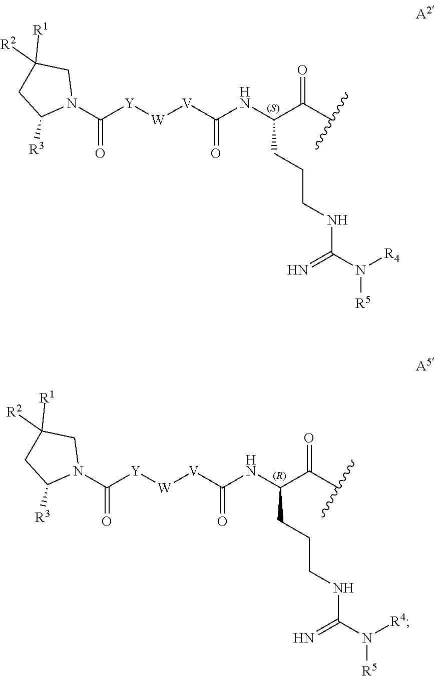

even more preferably A^{2′a} to A^{2′m}, most preferably A^{2′c}:

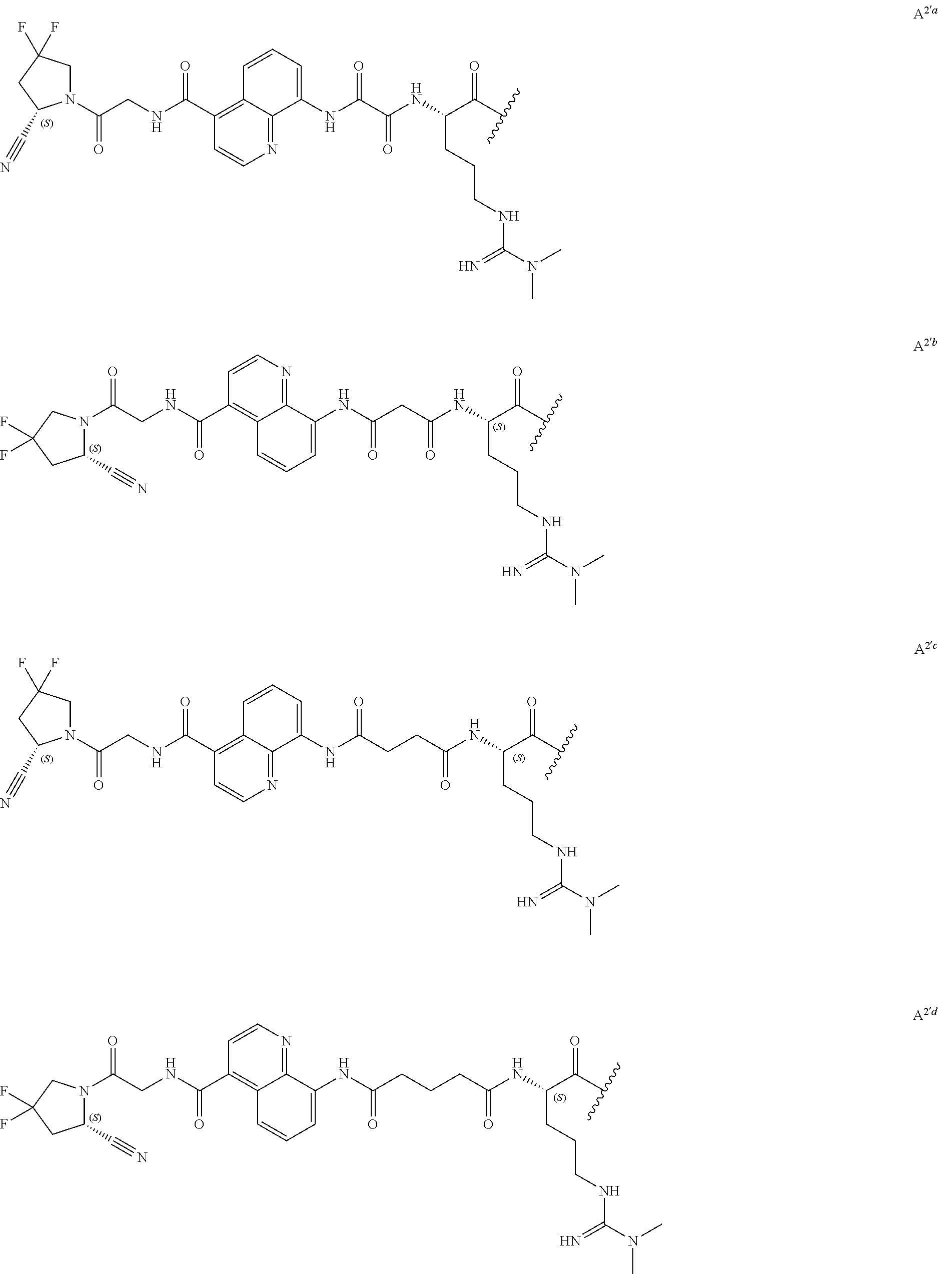

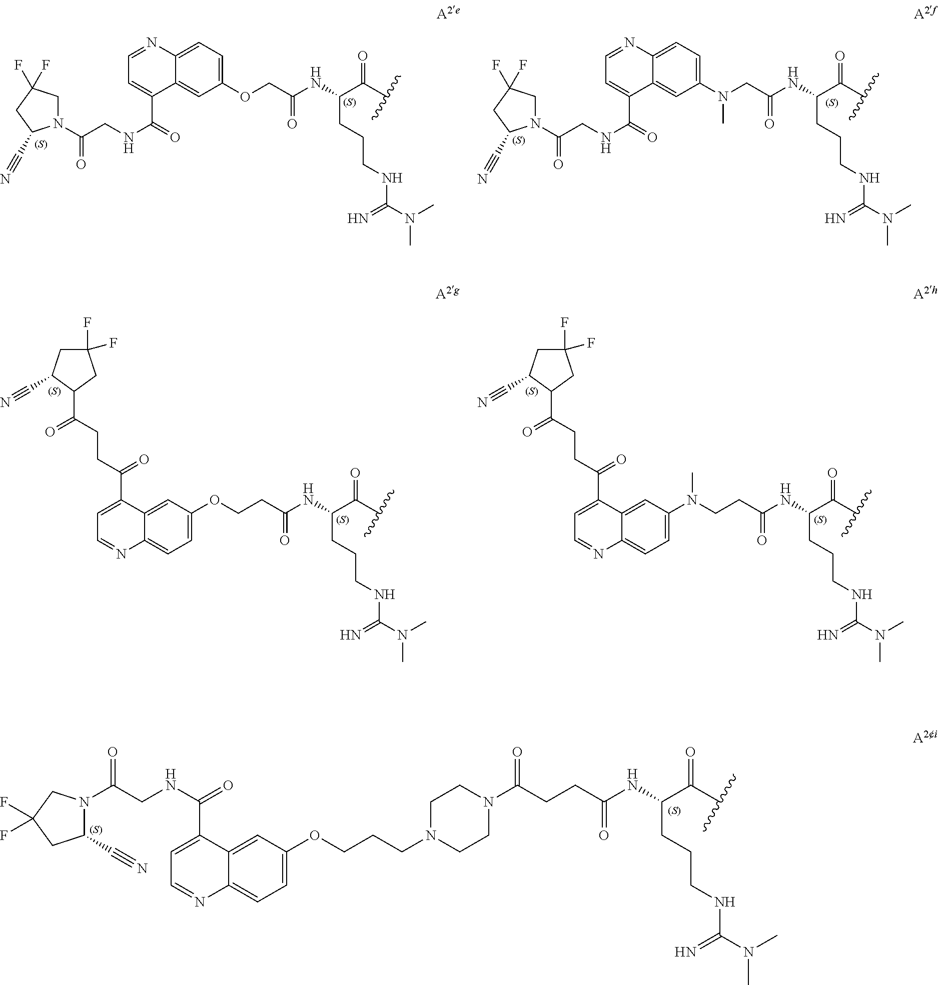

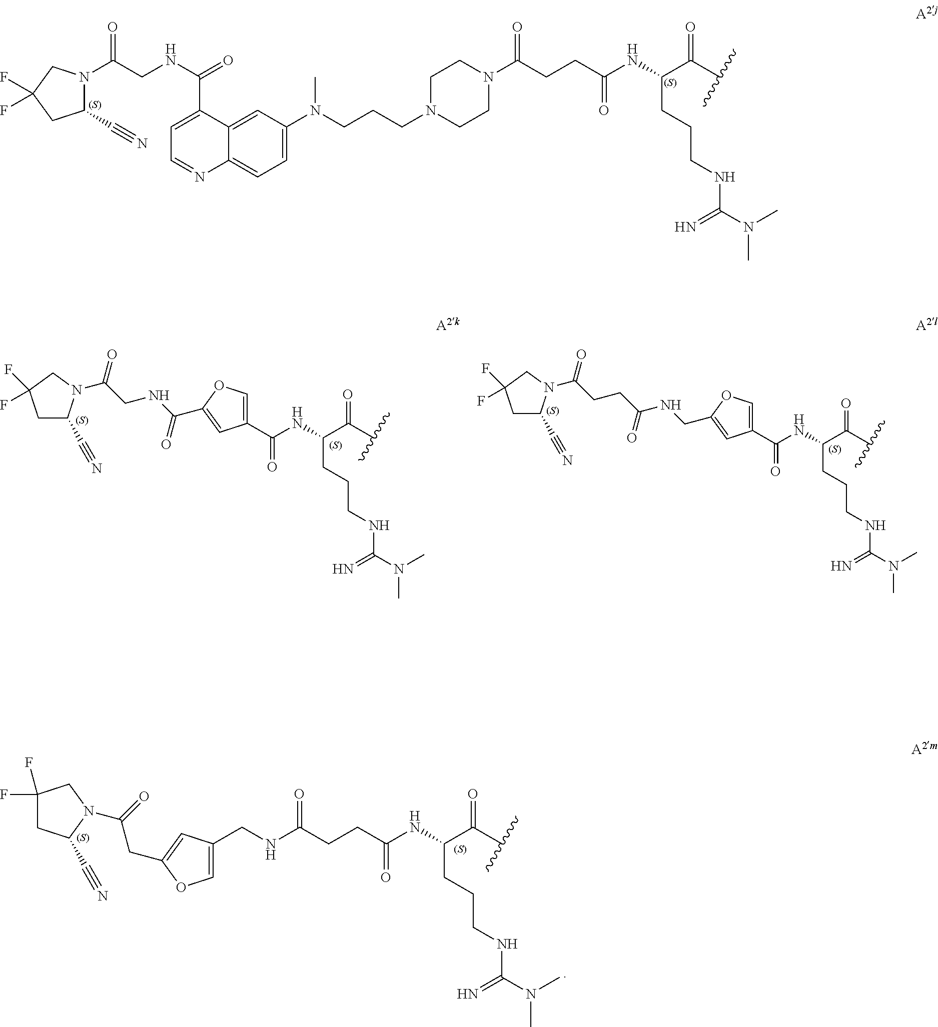

5. The compound of any one of the preceding embodiments, wherein Y and V are each independently selected from: single bond,

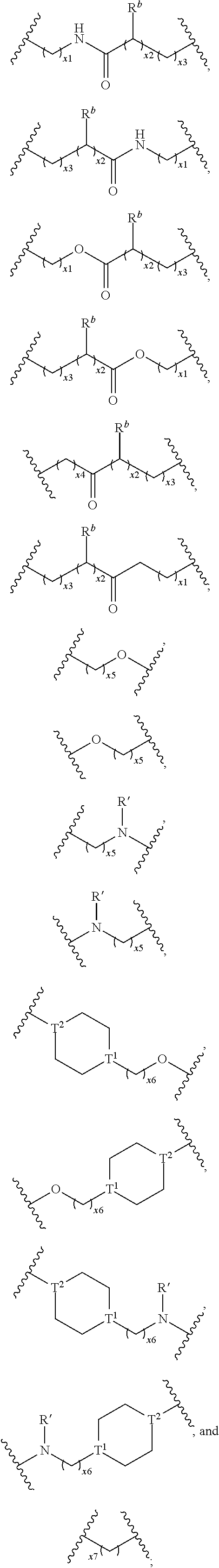

wherein R^{b} is H or an optionally substituted C₁₋₆ alkyl, C₂₋₆ alkenyl, C₂₋₆ alkynyl or C₆-C₁₀ aryl group, in which optionally one or more of the carbon atoms can be replaced by heteroatoms; preferably a side chain derived from a proteinogenic or non-proteinogenic amino acid; more preferably H, —CH₃, —CH₂OH, —CH₂CH₂SCH₃, —C₂H₅, —CH₂CH₂CH₃, —CH₂CH₂CH₂CH₃, —CH(CH₃)₂, or -Ph;
T¹ and T² are each independently selected from CR′ and N;
x1 is an integer of 0 to 14, preferably 0, 1, 2 or 3;
x2 is an integer of 0 to 6, preferably 0, 1, 2 or 3;
x3 is an integer of 0 to 14, preferably 0, 1, 2 or 3;
x4 is an integer of 0 to 14, preferably 0, 1, 2 or 3;
x5 is an integer of 0 to 15, preferably 0, 1, 2 or 3;
x6 is an integer of 0 to 11, preferably 0, 1, 2 or 3;
x7 is an integer of 1 to 16, preferably 1, 2 or 3;
x1+x2+x3 is 14 or less, preferably 0, 1, 2, 3 or 4;
x2+x3+x4 is 15 or less, preferably 0, 1, 2, 3, 4 or 5;
preferably wherein Y and V are each independently selected from: single bond,

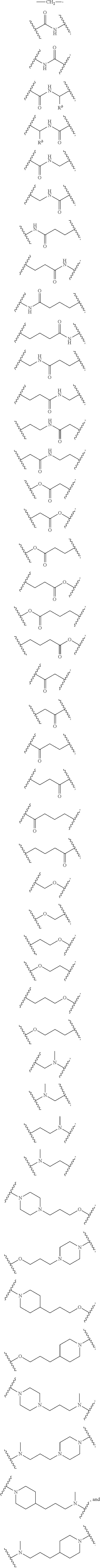

more preferably wherein:
Y is selected from: single bond,

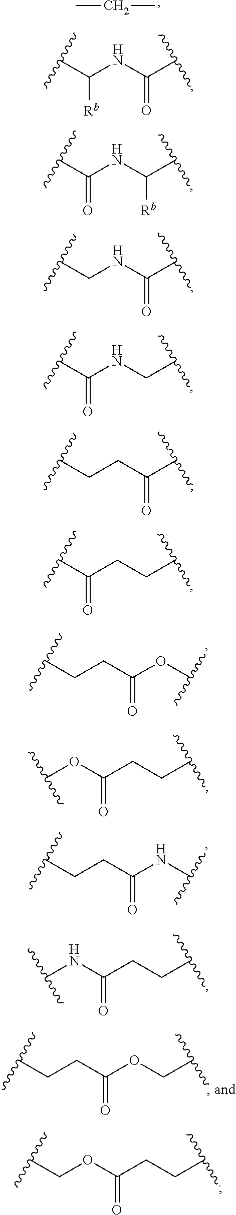

most preferably

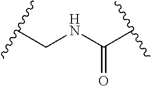 [SMILES: *CNC(*)=O]

or —CH₂—; and/or
V is selected from: single bond,

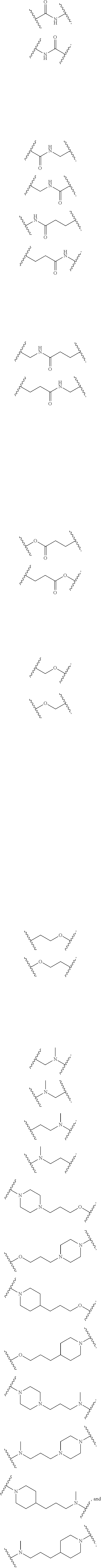

most preferably

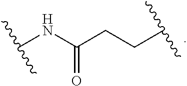 [SMILES: *CCC(=O)N*]

6. The compound of any one of the preceding embodiments, wherein W is monocyclic or bicyclic optionally substituted C₆-C₁₀ aryl, or a monocyclic or bicyclic optionally substituted 3- to 12 membered carbocyclyl or heterocyclyl group,
preferably monocyclic or bicyclic C₃₋₁₀ heteroaryl;
more preferably

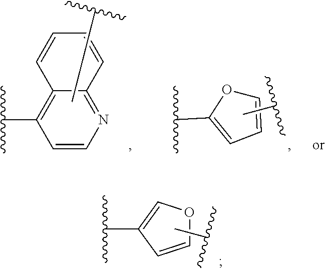

even more preferably

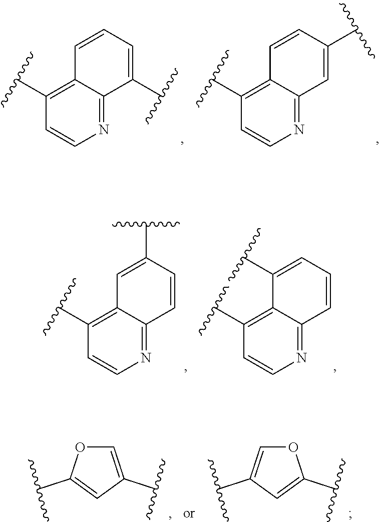

most preferably

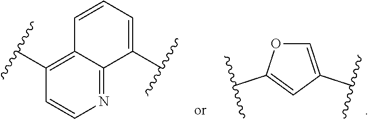 [SMILES: *c1ccnc2c(*)cccc12 ; *c1coc(*)c1]

7. The compound of any one of the preceding embodiments, wherein AA² is

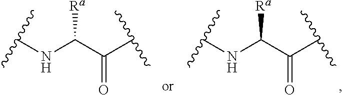

preferably wherein R^{a} is —CH₃, —CH(CH₃)₂, —CH₂C≡CH, —((CH)₂)₄NH₂ or —CH₂-Ph; more preferably wherein AA² is

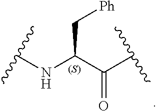

8. The compound of any one of the preceding embodiments, wherein the compound is represented by the following Formula I, Ia, or Tb,

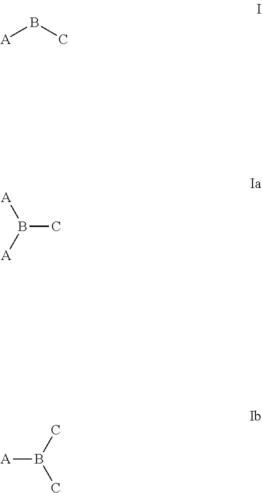

wherein each A is independently defined as in any one of the preceding embodiments;
B is a single bond or an optionally substituted C₁₋₅₀ aliphatic group, in which optionally one or more carbon atoms can be replaced by a heteroatom, a C₃₋₁₂ carbocyclic or a C₁₋₁₂ heterocyclic group, and which can be saturated optionally contain one or more double or triple bonds; and
each C is an atom, a molecule or a particle, and/or is a therapeutic or diagnostic agent.

9. The compound of embodiment 8, wherein B is represented by any of the following general Formulae II-V, Ha-Va, or IIb-Vb:

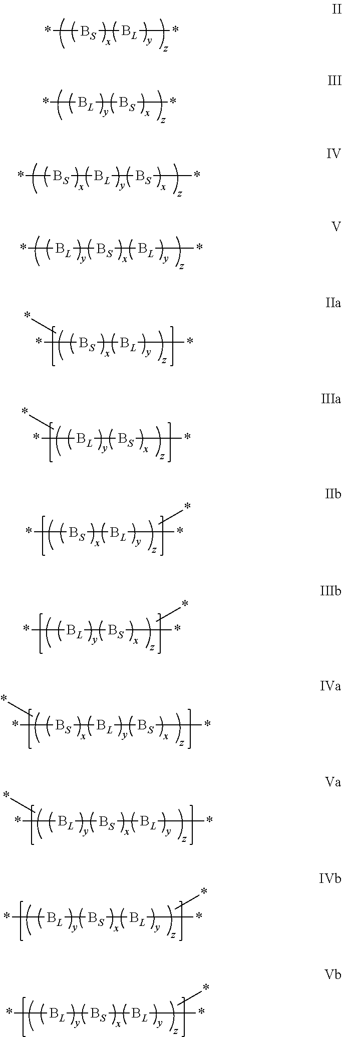

wherein
each x is 0, 1, 2, 3, 4, 5, 6, 7, 8, 9, or 10;
each y is 0, 1, 2, 3, 4, 5, 6, 7, 8, 9, or 10;
each z is 0, 1, 2, 3 or 4;
with the proviso that in Formulae IIa-Va and IIb-Vb, z and at least one of x and y is not 0;
* represents a point of attachment to a moiety A;
* represents a point of attachment to a moiety C; and
each of B_{S} and B_{L} is independently selected from alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, aminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide, tetrapeptide, each of which is optionally substituted.

10. The compound of any one of the preceding embodiments, wherein each B_{S} and B_{L} is independently selected from:

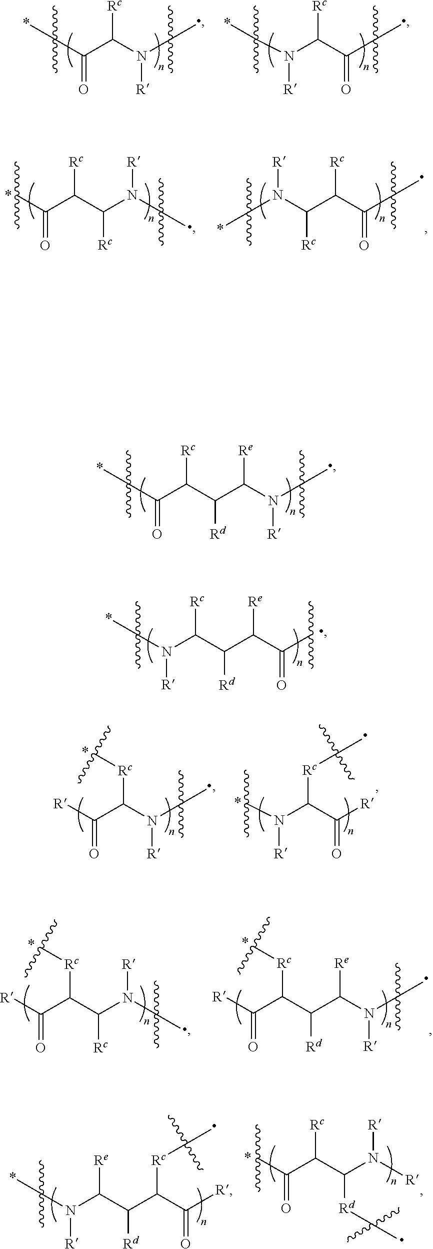

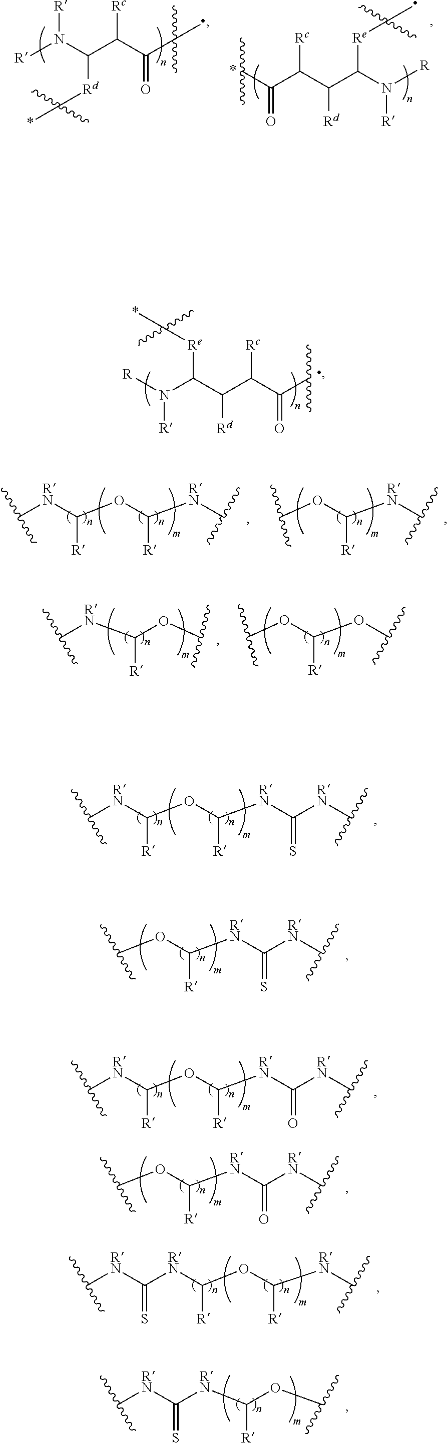

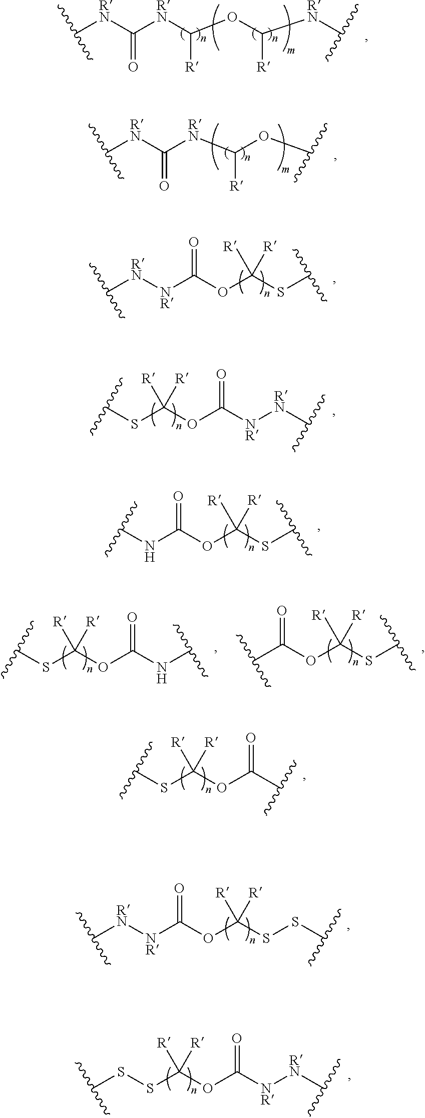

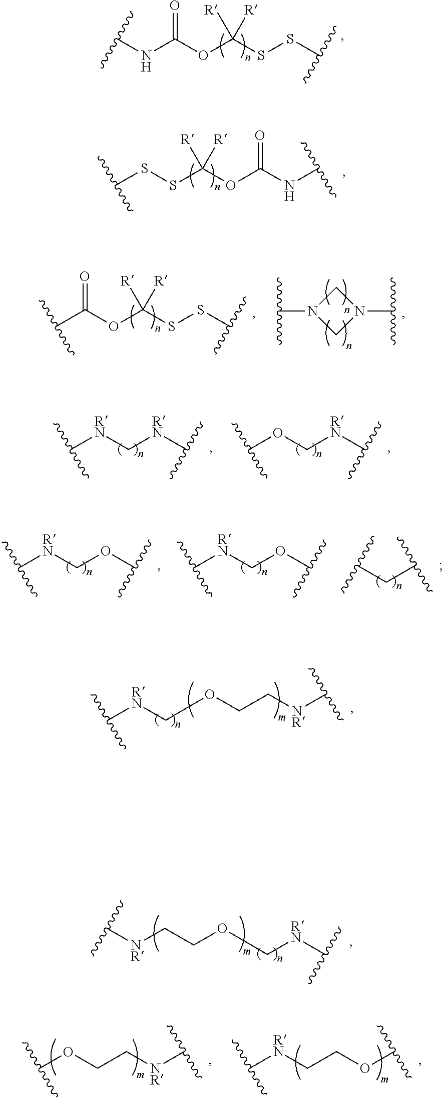

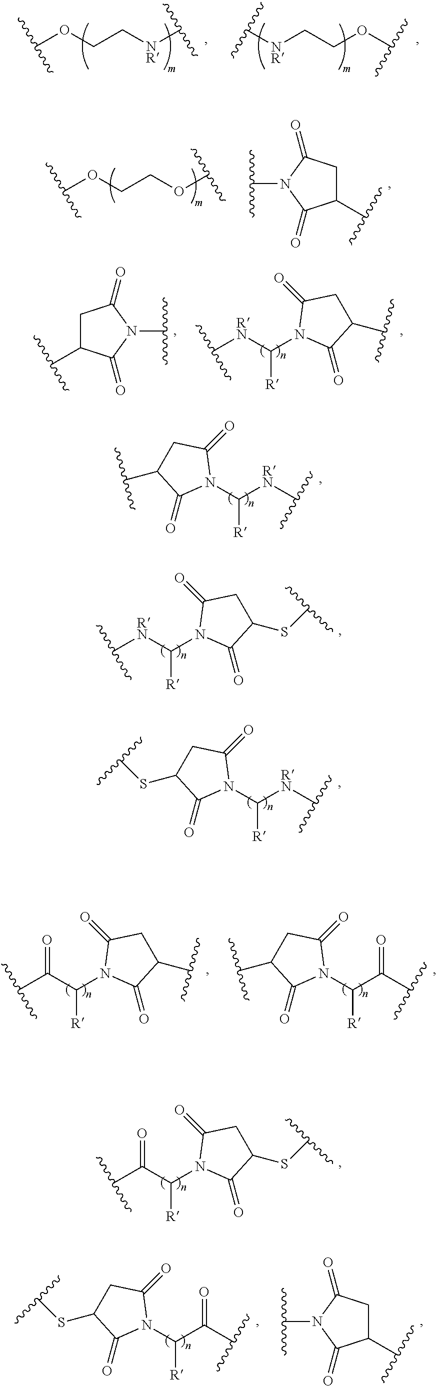

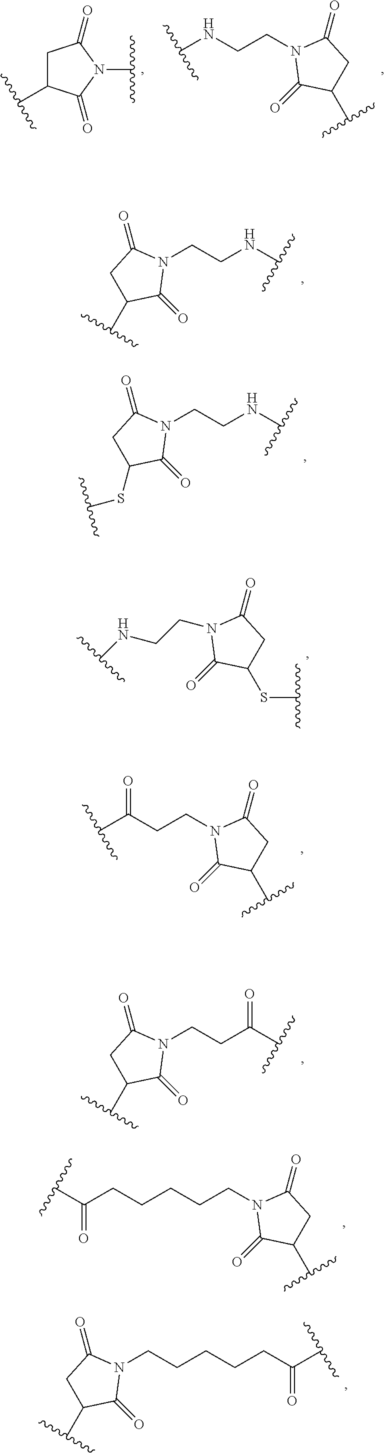

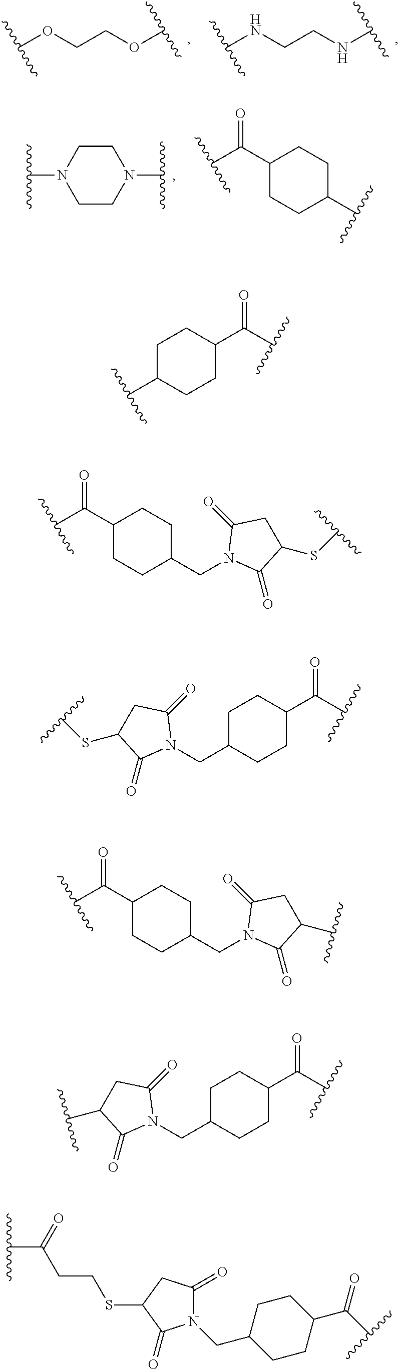

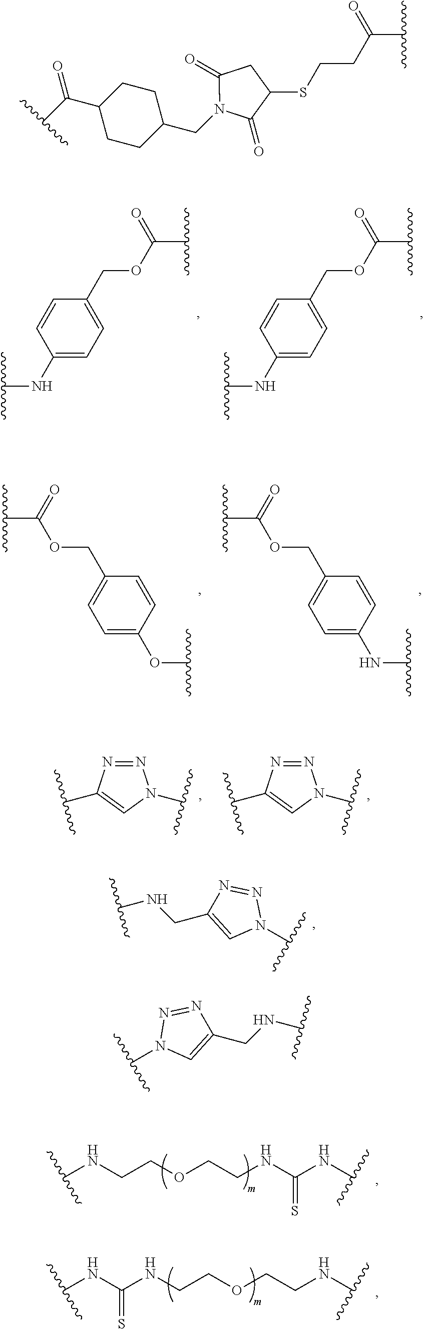

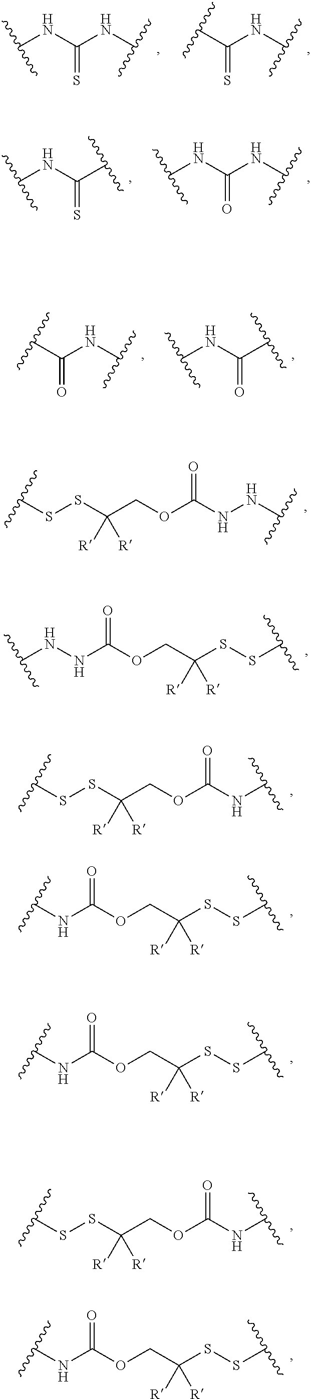

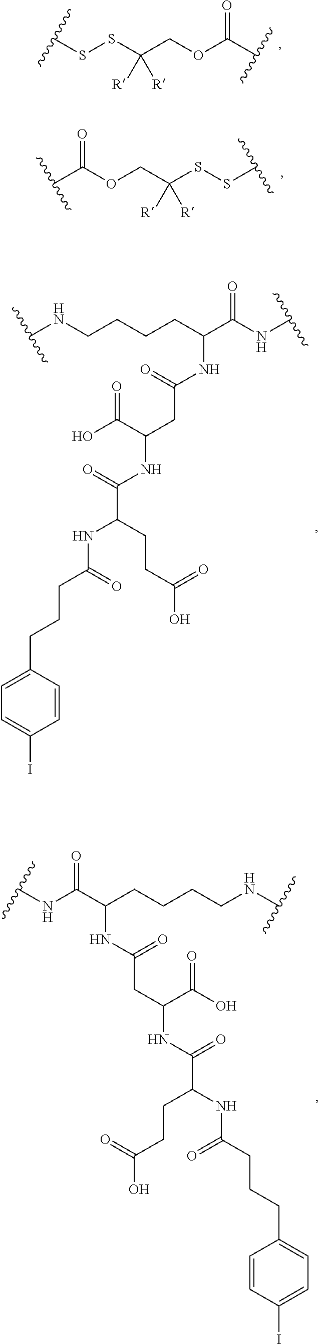

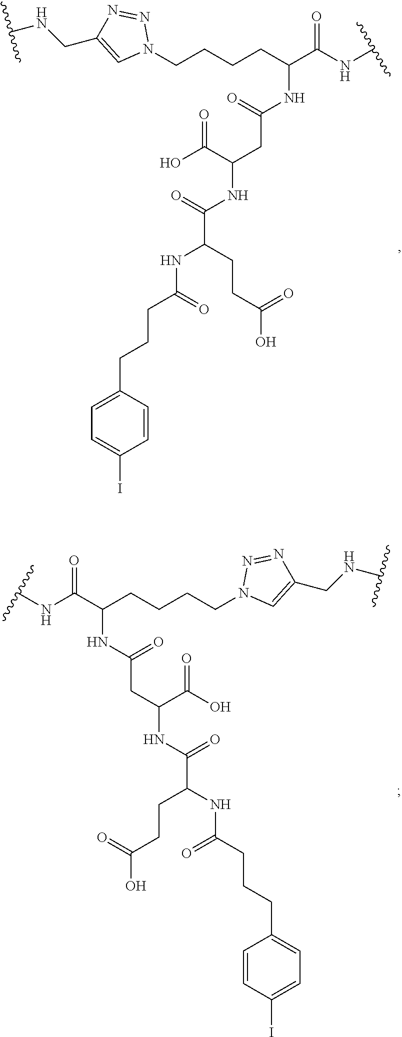 [SMILES: *NCc1cn(CCCCC(NC(=O)CC(NC(=O)C(CCC(=O)O)NC(=O)CCCc2ccc(I)cc2)C(=O)O)C(=O)N*)nn1 ; *NCc1cn(CCCCC(NC(=O)CC(NC(=O)C(CCC(=O)O)NC(=O)CCCc2ccc(I)cc2)C(=O)O)C(=O)N*)nn1]

wherein in each of the above structures:
each n is independently 0, 1, 2, 3, 4, 5, 6, 7, or 8;
each m is independently 0, 1, 2, 3, or 4;
each R^{c}, R^{d}, and R^{e} is independently is selected from H, optionally substituted C₁₋₆ alkyl, (C₃-C₁₀ carbocyclyl)C₁₋₆ alkyl, (C₆-C₁₀ aryl)C₁₋₆ alkyl, (C₁-C₁₀ heterocyclyl)C₁₋₆ alkyl, C₂₋₆ alkenyl, C₂₋₆ alkynyl, and C₆-C₁₀ aryl, in each of which optionally one or more of the carbon atoms can be replaced by heteroatoms; preferably selected from side-chain residues of proteinogenic or a non-proteinogenic amino acids;
each * represents a point of attachment for which the shortest path to a moiety A comprises less atoms than that for •; and each • represents a point of attachment for which the shortest path to a moiety C comprises less atoms than that for *, with the proviso that when n is >1 and a respective point of attachment is indicated on any one of R^{c}, R^{d} and R^{e}, then it can be independently present in one or more of the peptide monomeric units, preferably in one peptide monomeric unit most distant from the other point of attachment indicated in the respective structure. wherein each of the above structures optionally comprises a further attachment point to a moiety A or C.

11. The compound according to any one of the preceding embodiments, wherein moiety B has one of the following structures:
(a) single bond,

(B_{S})_{X},

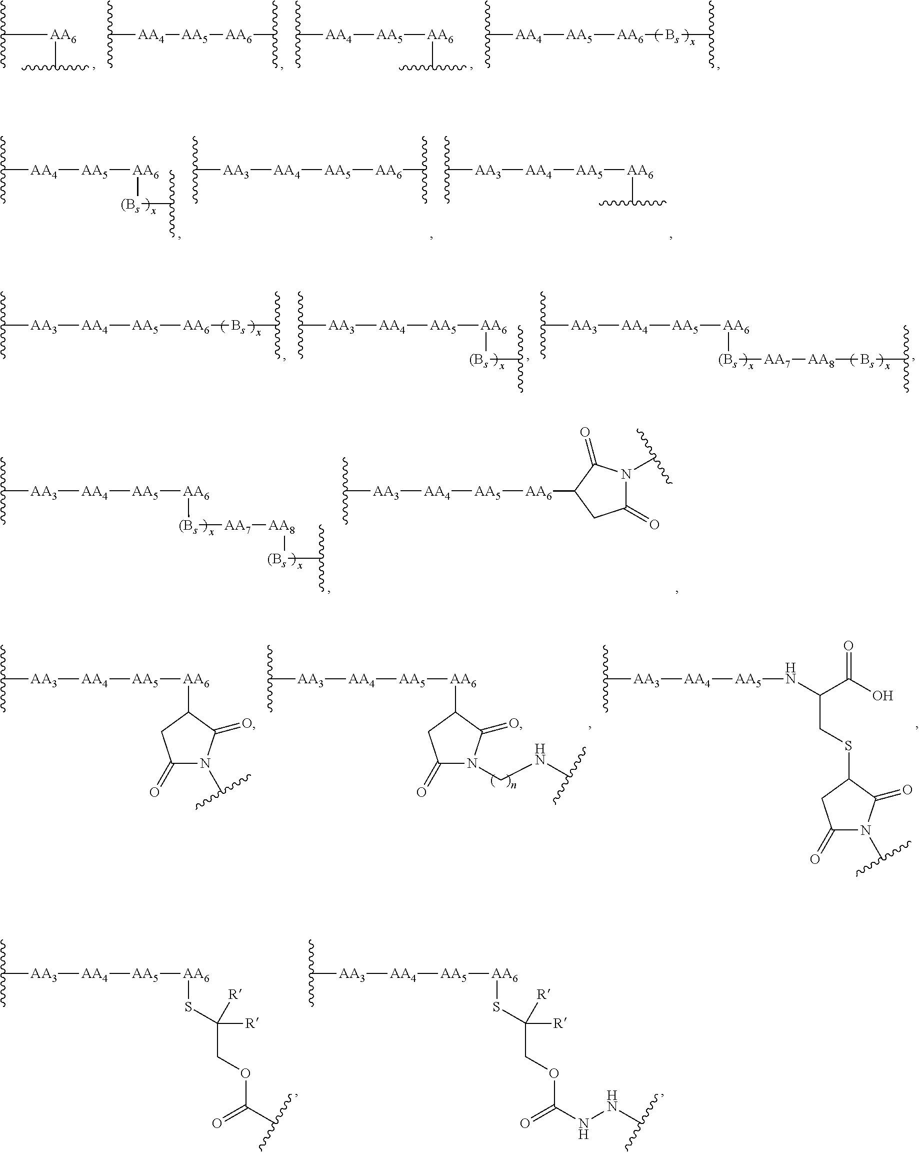

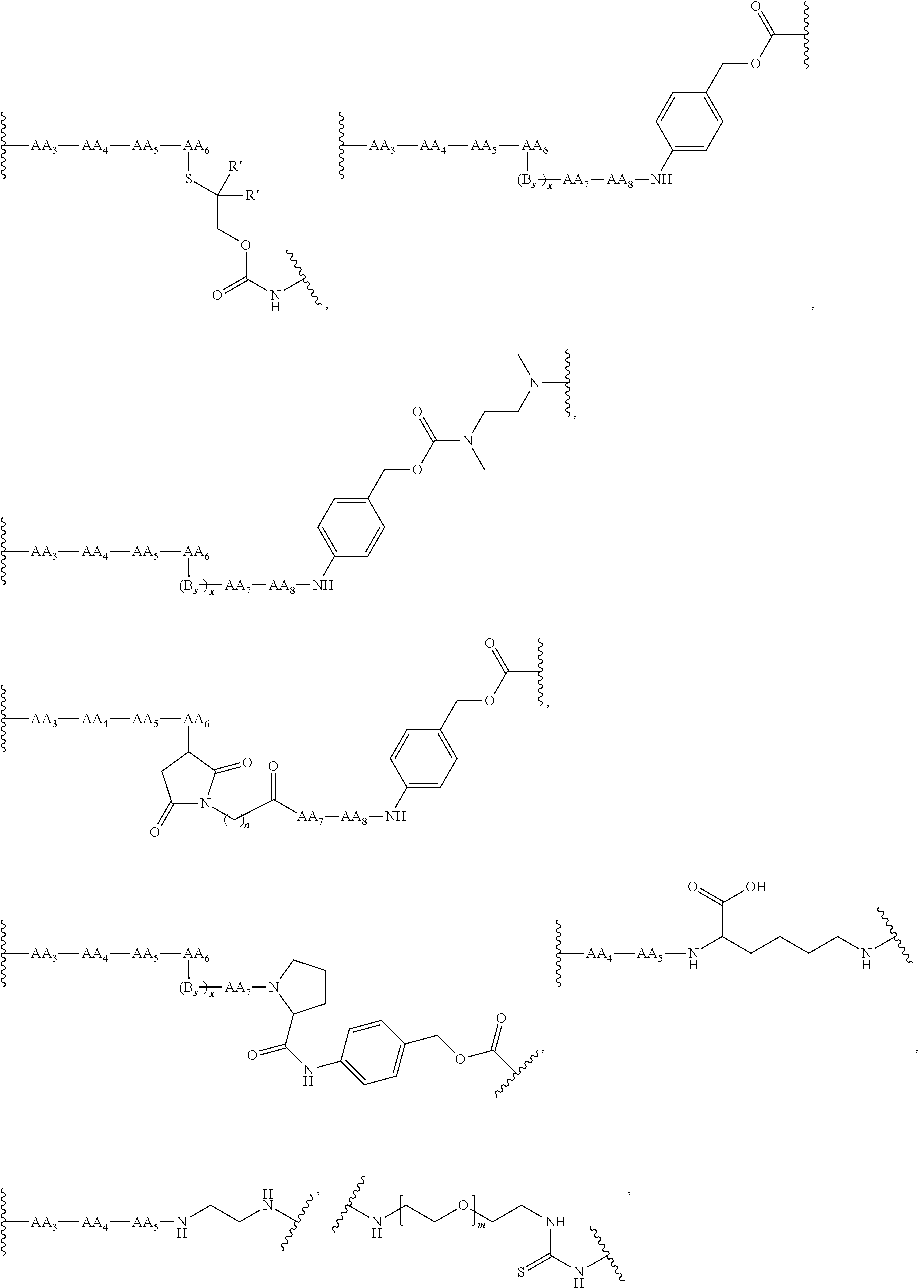

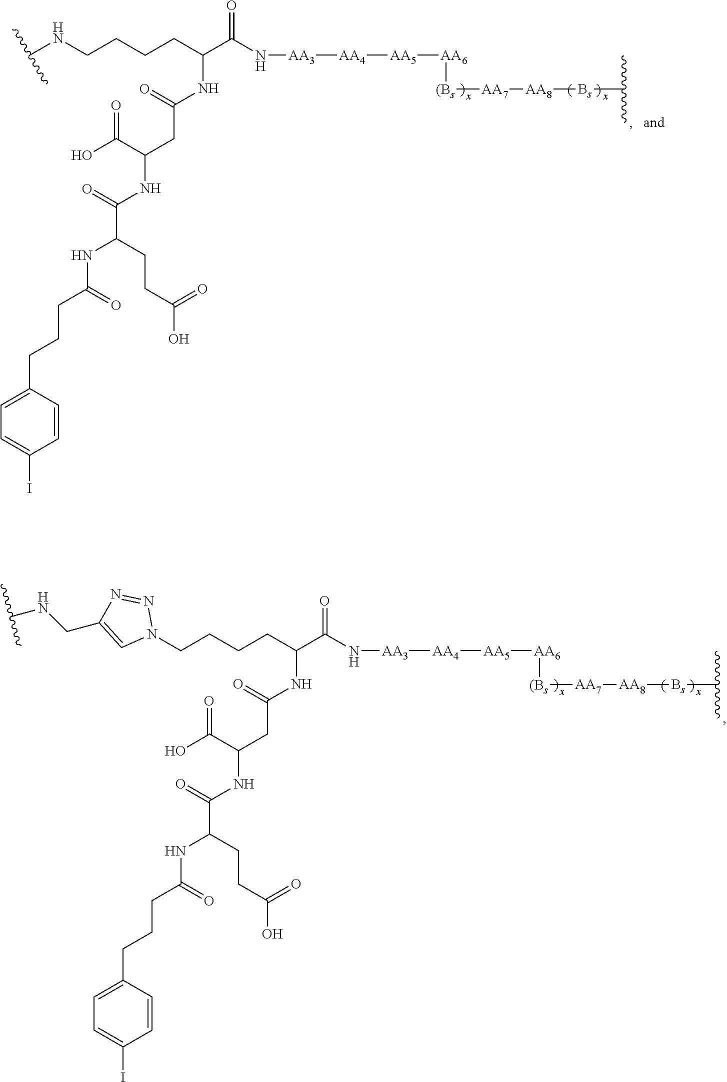

wherein each of AA₃, AA₄, AA₅, AA₆, AA₇, and AA₈ represents a proteinogenic or non-proteinogenic amino acid, or is absent;
wherein preferably:
   each proteinogenic or non-proteinogenic amino acid is preferably independently represented by one of the following structures:

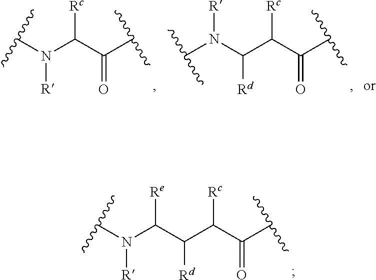

and/or AA₄ is an amino acid with a charged sidechain, and AA₇ is an amino acid with an aliphatic sidechain;
wherein more preferably:
   AA₃ is selected from Asp, Glu, and Lys, or is absent; preferably Asp;
   AA₄ is selected from Arg, HomoArg, Lys, Asp, and Glu, or is absent; preferably Lys or Arg;
   AA₅ is selected from Asp, Glu, and Lys; preferably Asp;
   AA₆ is selected from Cys, Lys, Gly and Val; preferably Cys or Lys;
   AA₇ is selected from Gly, Ala, Val, Arg, Ile, Pro; and
   AA₈ is selected from Pro and citrulline (Cit);
even more preferably according to one of the sequences shown in the below table:

| AA₃ | AA₄ | AA₅ | AA₆ | AA₇ | AA₈ | AA₃ | AA₄ | AA₅ | AA₆ | AA₇ | AA₈ |
|---|---|---|---|---|---|---|---|---|---|---|---|
| — | — | — | Lys | — | — | | | | | | |
| Asp | Arg | Asp | Cys | — | — | Asp | Lys | Asp | Cys | Ala | Pro |
| Asp | Arg | Asp | Gly | — | — | Asp | Lys | Asp | Cys | Ala | Val |
| Asp | Lys | Asp | Lys | — | — | Asp | Lys | Asp | Cys | Arg | Pro |
| Asp | Lys | Asp | Val | — | — | Asp | Lys | Asp | Cys | Gly | Pro |
| Asp | Arg | Asp | Cys | Val | Ala | Asp | Lys | Asp | Cys | Ile | Pro |
| Asp | Arg | Asp | Cys | Val | Cit | Asp | Lys | Asp | Cys | Pro | Pro |
| Asp | HomoArg | Asp | Cys | Val | Cit | Asp | Lys | Asp | Cys | Val | Cit |
| Asp | Lys | Asp | Cys | any AA | Pro | Asp | Lys | Asp | Cys | Val | Pro |

or
(b) single bond,

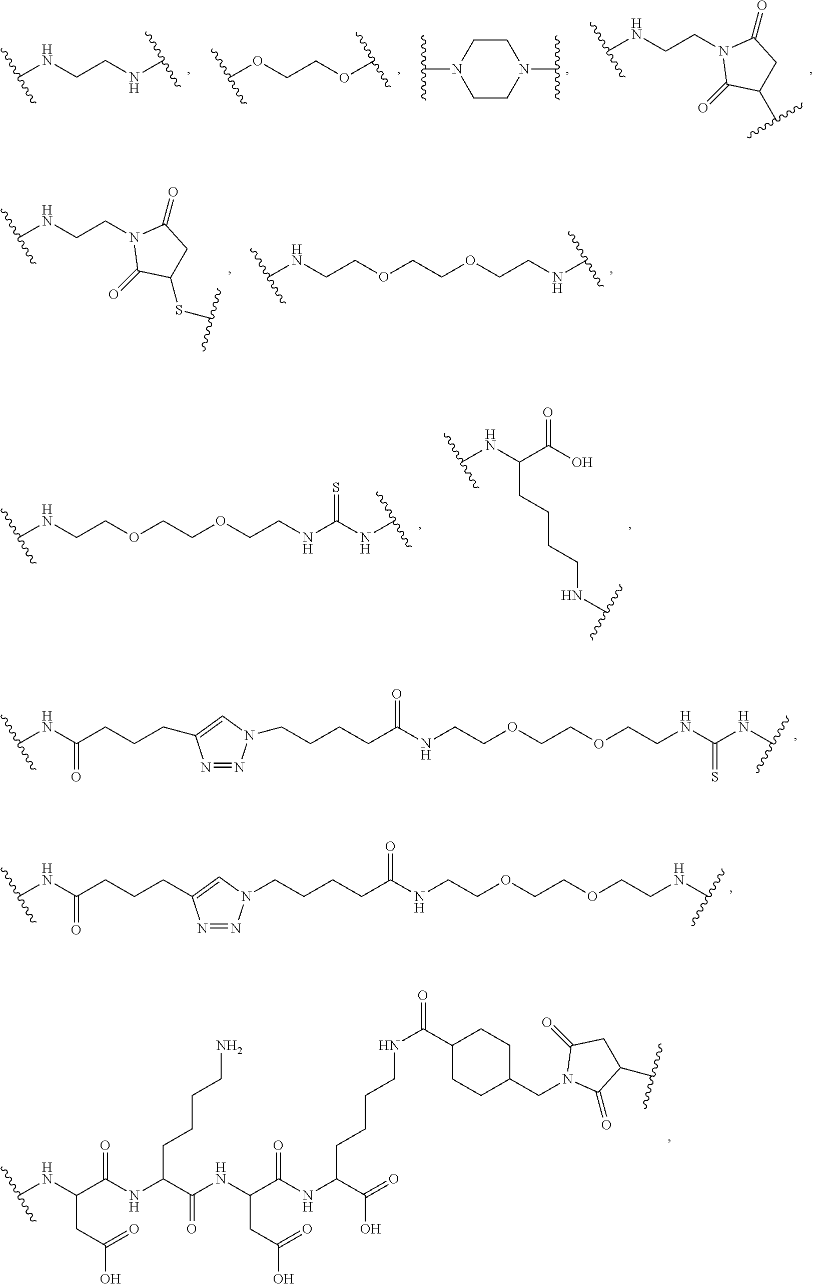

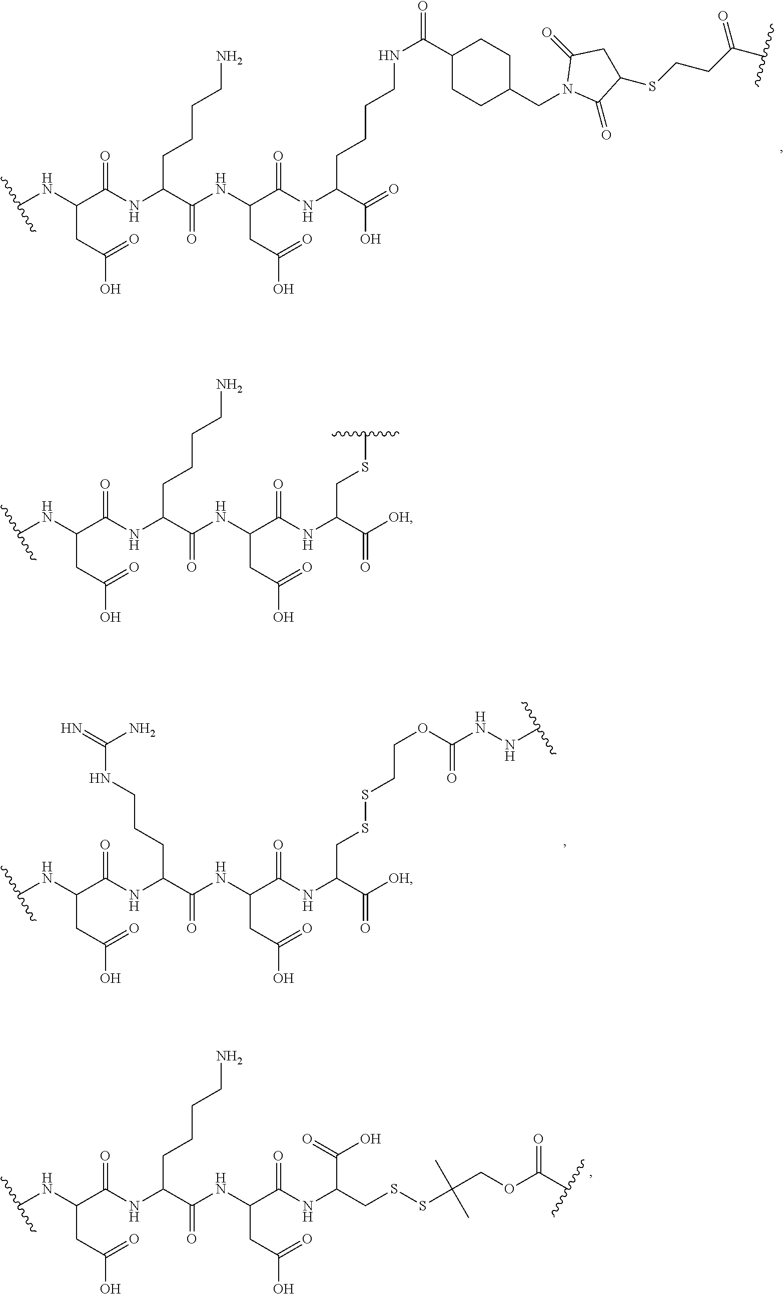 [SMILES: *NC(CC(=O)O)C(=O)NC(CCCCN)C(=O)NC(CC(=O)O)C(=O)NC(CCCCNC(=O)C1CCC(CN2C(=O)CC(SCCC(*)=O)C2=O)CC1)C(=O)O ; *NC(CC(=O)O)C(=O)NC(CCCCN)C(=O)NC(CC(=O)O)C(=O)NC(CS*)C(=O)O ; *NNC(=O)OCCSSCC(NC(=O)C(CC(=O)O)NC(=O)C(CCCNC(=N)N)NC(=O)C(CC(=O)O)N*)C(=O)O ; *NC(CC(=O)O)C(=O)NC(CCCCN)C(=O)NC(CC(=O)O)C(=O)NC(CSSC(C)(C)COC(*)=O)C(=O)O]

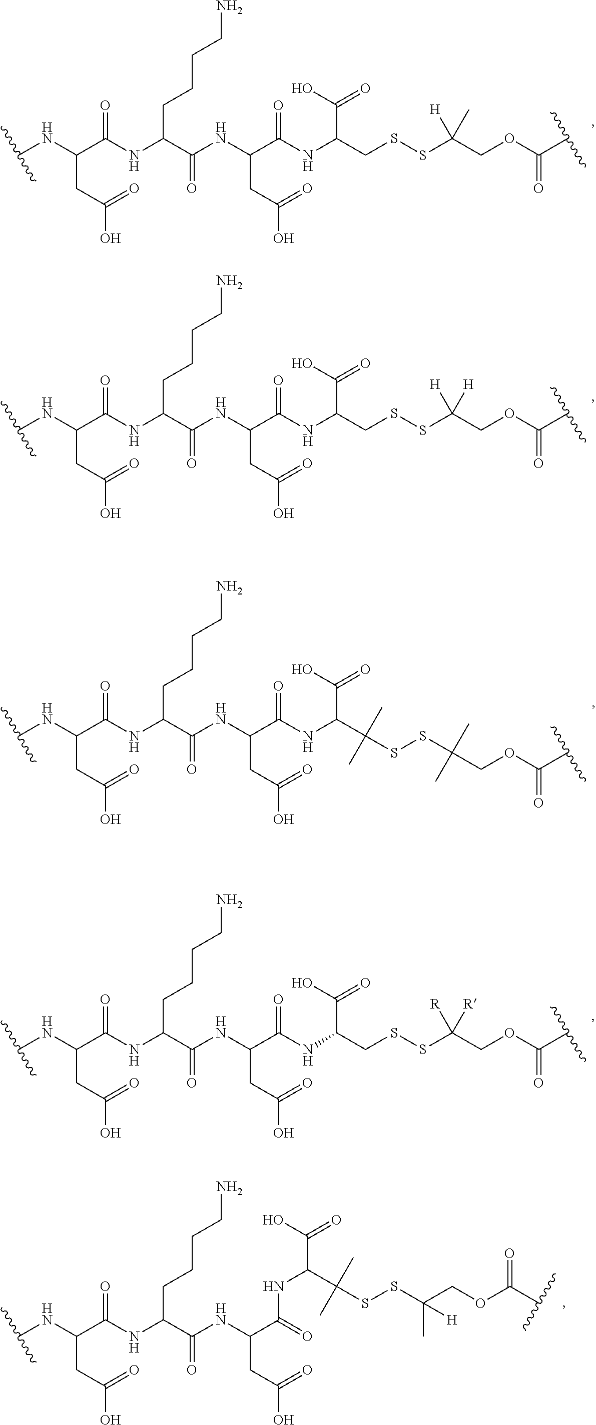

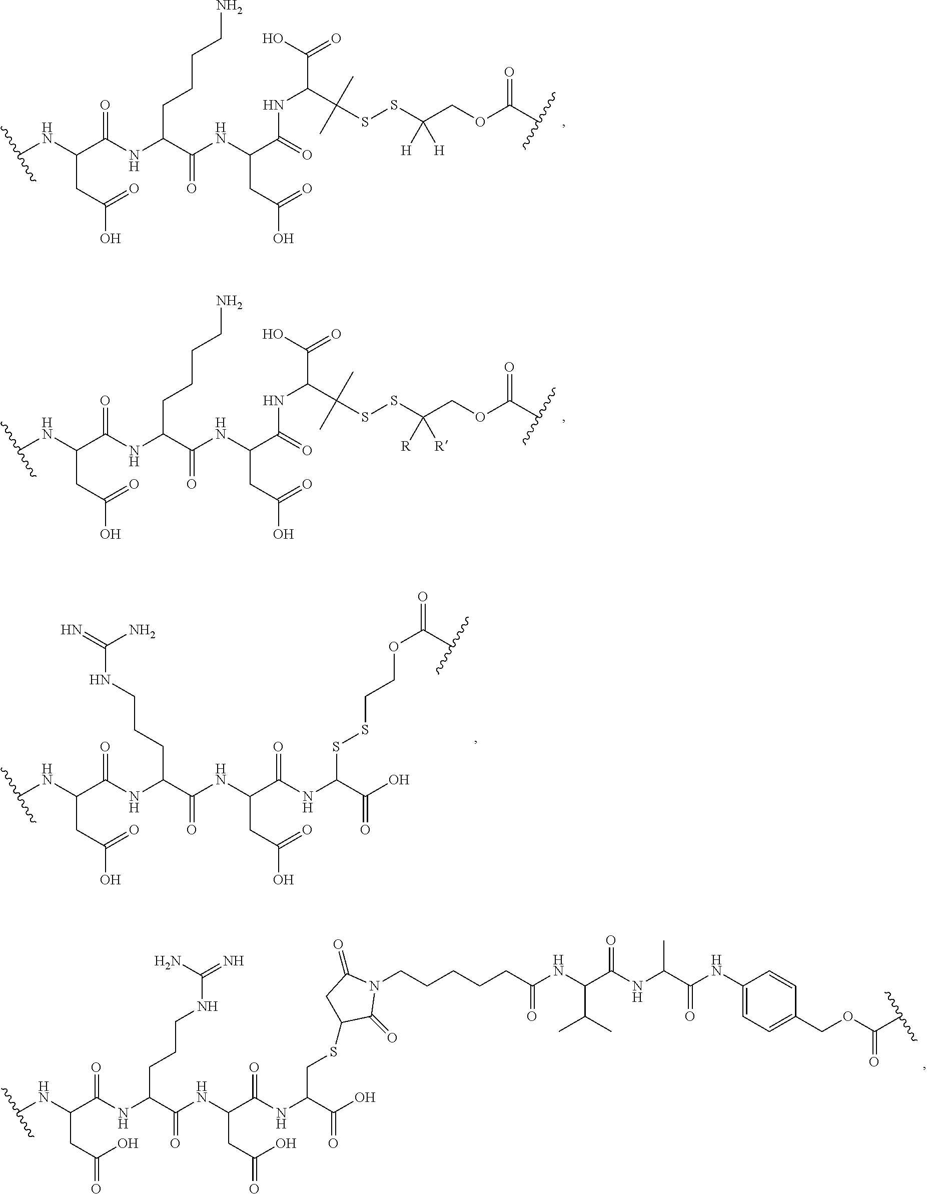

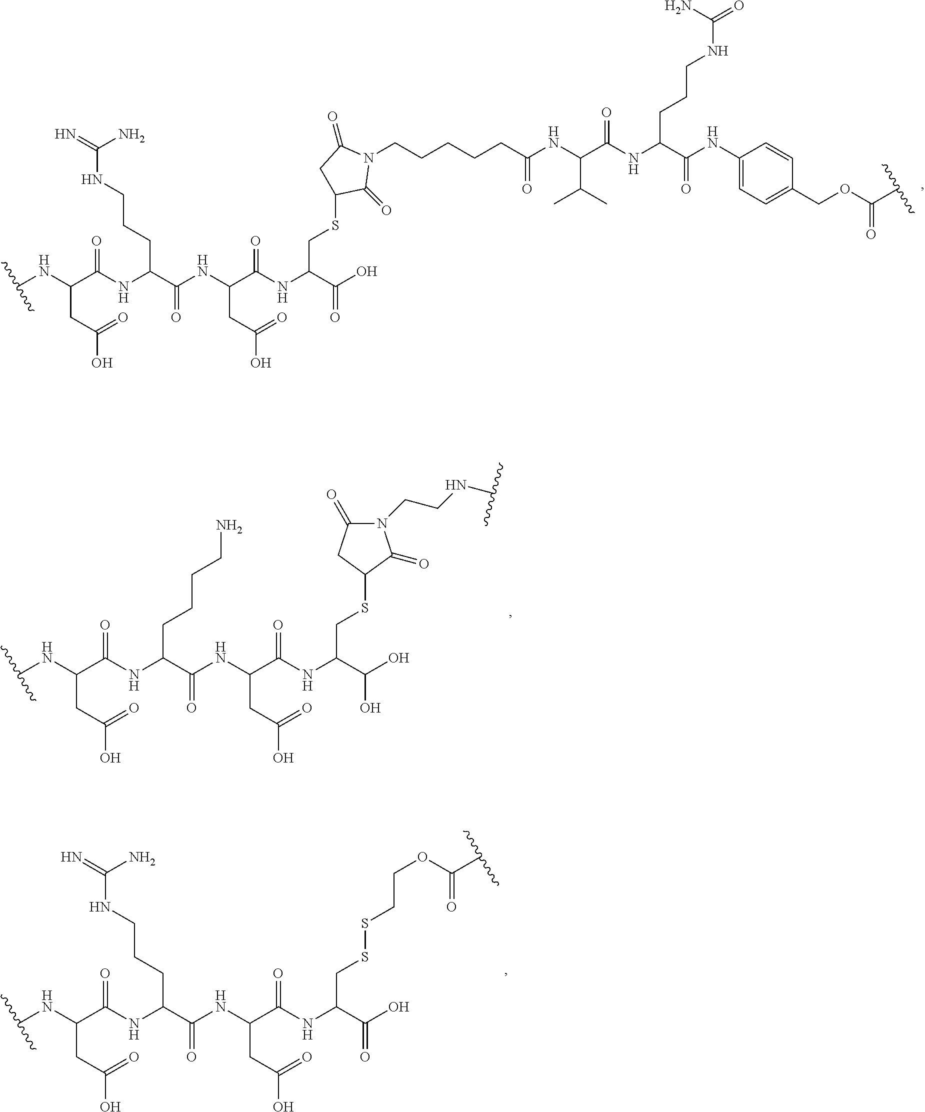 [SMILES: *NC(CC(=O)O)C(=O)NC(CCCNC(=N)N)C(=O)NC(CC(=O)O)C(=O)NC(CSC1CC(=O)N(CCCCCC(=O)NC(C(=O)NC(CCCNC(N)=O)C(=O)Nc2ccc(COC(*)=O)cc2)C(C)C)C1=O)C(=O)O ; *NCCN1C(=O)CC(SCC(NC(=O)C(CC(=O)O)NC(=O)C(CCCCN)NC(=O)C(CC(=O)O)N*)C(O)O)C1=O ; *NC(CC(=O)O)C(=O)NC(CCCNC(=N)N)C(=O)NC(CC(=O)O)C(=O)NC(CSSCCOC(*)=O)C(=O)O]

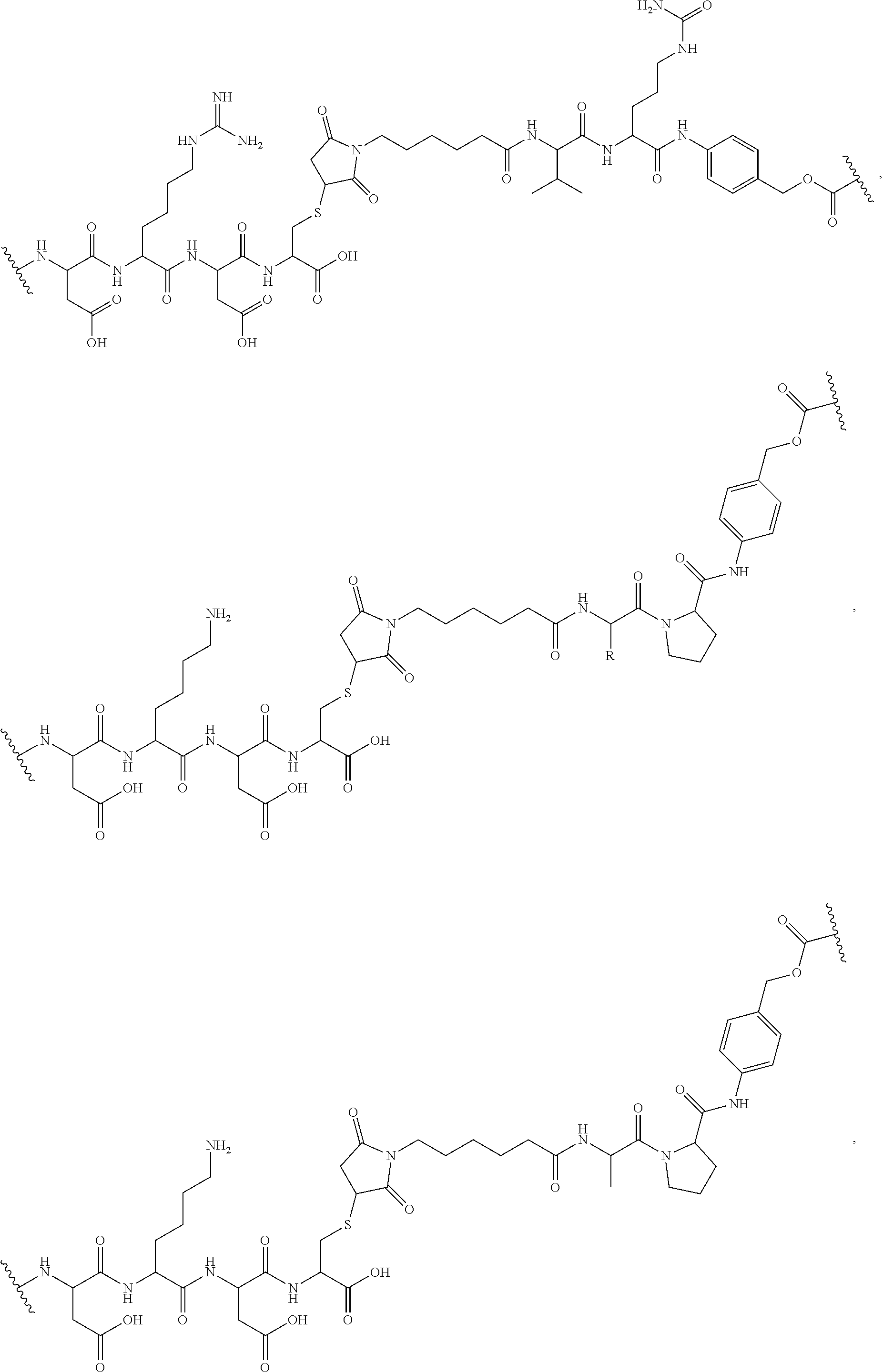

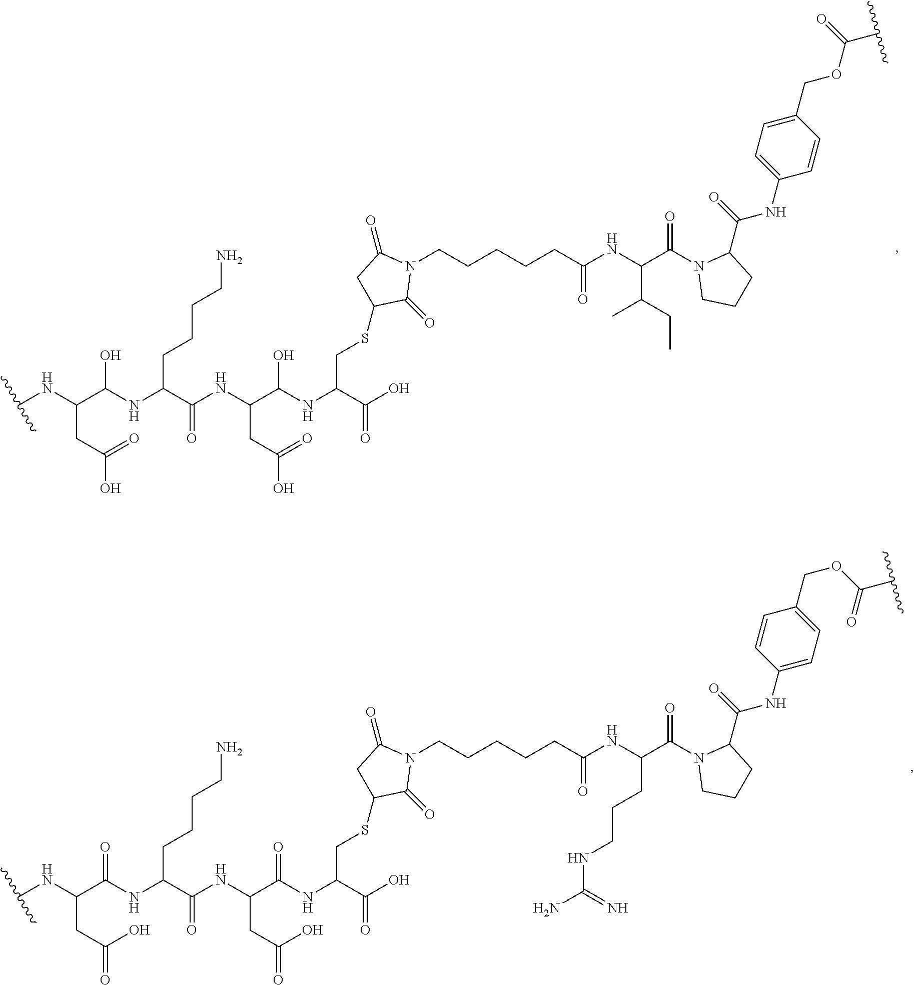 [SMILES: *NC(CC(=O)O)C(O)NC(CCCCN)C(=O)NC(CC(=O)O)C(O)NC(CSC1CC(=O)N(CCCCCC(=O)NC(C(=O)N2CCCC2C(=O)Nc2ccc(COC(*)=O)cc2)C(C)CC)C1=O)C(=O)O ; *NC(CC(=O)O)C(=O)NC(CCCCN)C(=O)NC(CC(=O)O)C(=O)NC(CSC1CC(=O)N(CCCCCC(=O)NC(CCCNC(=N)N)C(=O)N2CCCC2C(=O)Nc2ccc(COC(*)=O)cc2)C1=O)C(=O)O]

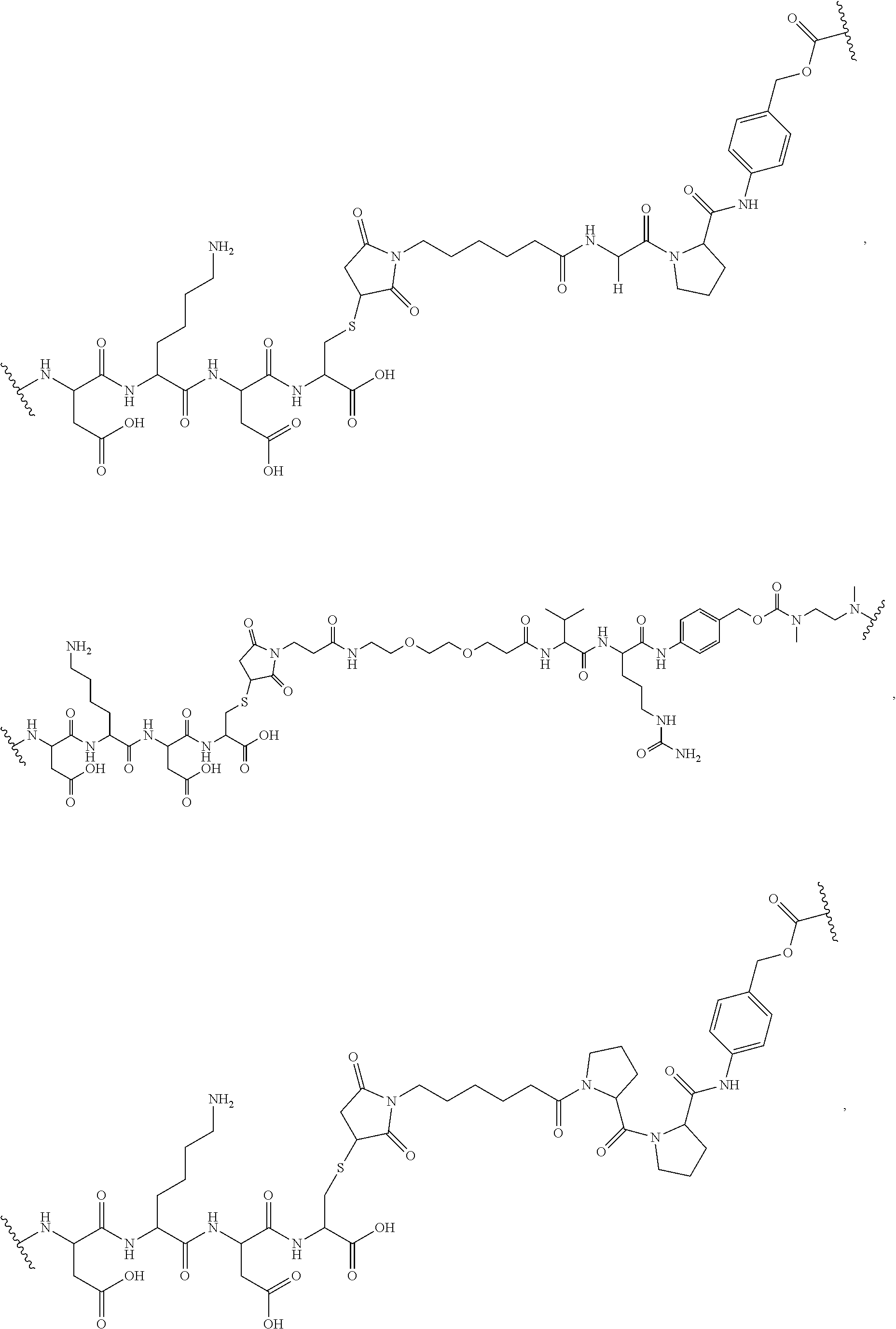 [SMILES: *NC(CC(=O)O)C(=O)NC(CCCCN)C(=O)NC(CC(=O)O)C(=O)NC(CSC1CC(=O)N(CCCCCC(=O)NCC(=O)N2CCCC2C(=O)Nc2ccc(COC(*)=O)cc2)C1=O)C(=O)O ; *NC(CC(=O)O)C(=O)NC(CCCCN)C(=O)NC(CC(=O)O)C(=O)NC(CSC1CC(=O)N(CCC(=O)NCCOCCOCCC(=O)NC(C(=O)NC(CCCNC(N)=O)C(=O)Nc2ccc(COC(=O)N(C)CCN(*)C)cc2)C(C)C)C1=O)C(=O)O ; *NC(CC(=O)O)C(=O)NC(CCCCN)C(=O)NC(CC(=O)O)C(=O)NC(CSC1CC(=O)N(CCCCCC(=O)N2CCCC2C(=O)N2CCCC2C(=O)Nc2ccc(COC(*)=O)cc2)C1=O)C(=O)O]

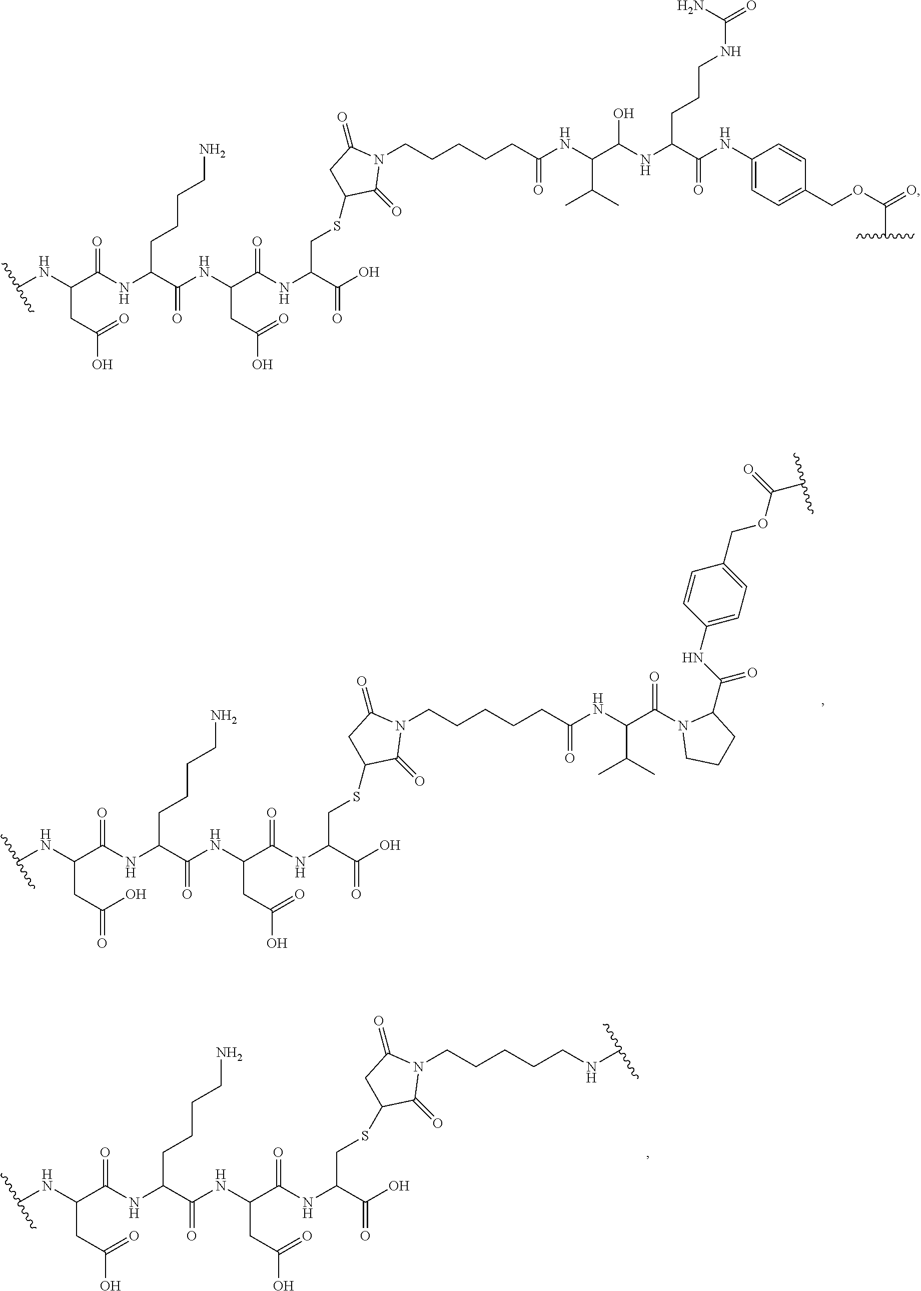 [SMILES: *NC(CC(=O)O)C(=O)NC(CCCCN)C(=O)NC(CC(=O)O)C(=O)NC(CSC1CC(=O)N(CCCCCC(=O)NC(C(C)C)C(O)NC(CCCNC(N)=O)C(=O)Nc2ccc(COC(*)=O)cc2)C1=O)C(=O)O ; *NC(CC(=O)O)C(=O)NC(CCCCN)C(=O)NC(CC(=O)O)C(=O)NC(CSC1CC(=O)N(CCCCCC(=O)NC(C(=O)N2CCCC2C(=O)Nc2ccc(COC(*)=O)cc2)C(C)C)C1=O)C(=O)O ; *NCCCCCN1C(=O)CC(SCC(NC(=O)C(CC(=O)O)NC(=O)C(CCCCN)NC(=O)C(CC(=O)O)N*)C(=O)O)C1=O]

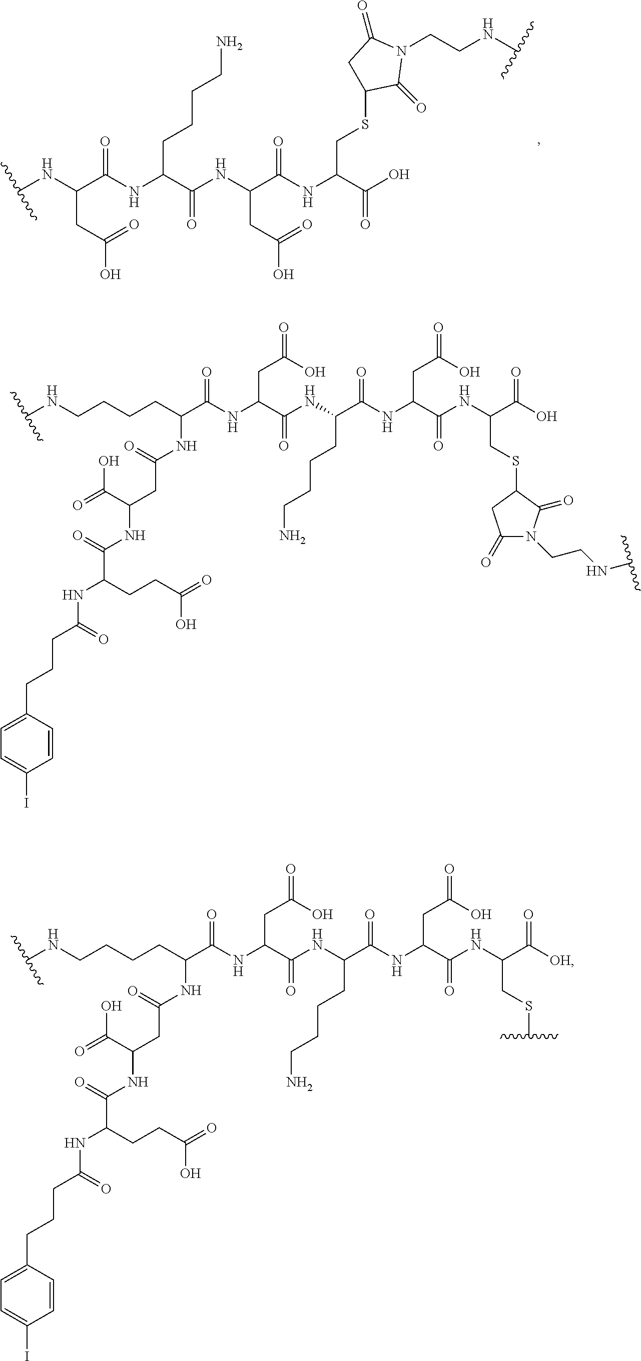 [SMILES: *NCCN1C(=O)CC(SCC(NC(=O)C(CC(=O)O)NC(=O)C(CCCCN)NC(=O)C(CC(=O)O)N*)C(=O)O)C1=O ; *NCCCCC(NC(=O)CC(NC(=O)C(CCC(=O)O)NC(=O)CCCc1ccc(I)cc1)C(=O)O)C(=O)NC(CC(=O)O)C(=O)N[C@@H](CCCCN)C(=O)NC(CC(=O)O)C(=O)NC(CSC1CC(=O)N(CCN*)C1=O)C(=O)O ; *NCCCCC(NC(=O)CC(NC(=O)C(CCC(=O)O)NC(=O)CCCc1ccc(I)cc1)C(=O)O)C(=O)NC(CC(=O)O)C(=O)NC(CCCCN)C(=O)NC(CC(=O)O)C(=O)NC(CS*)C(=O)O]

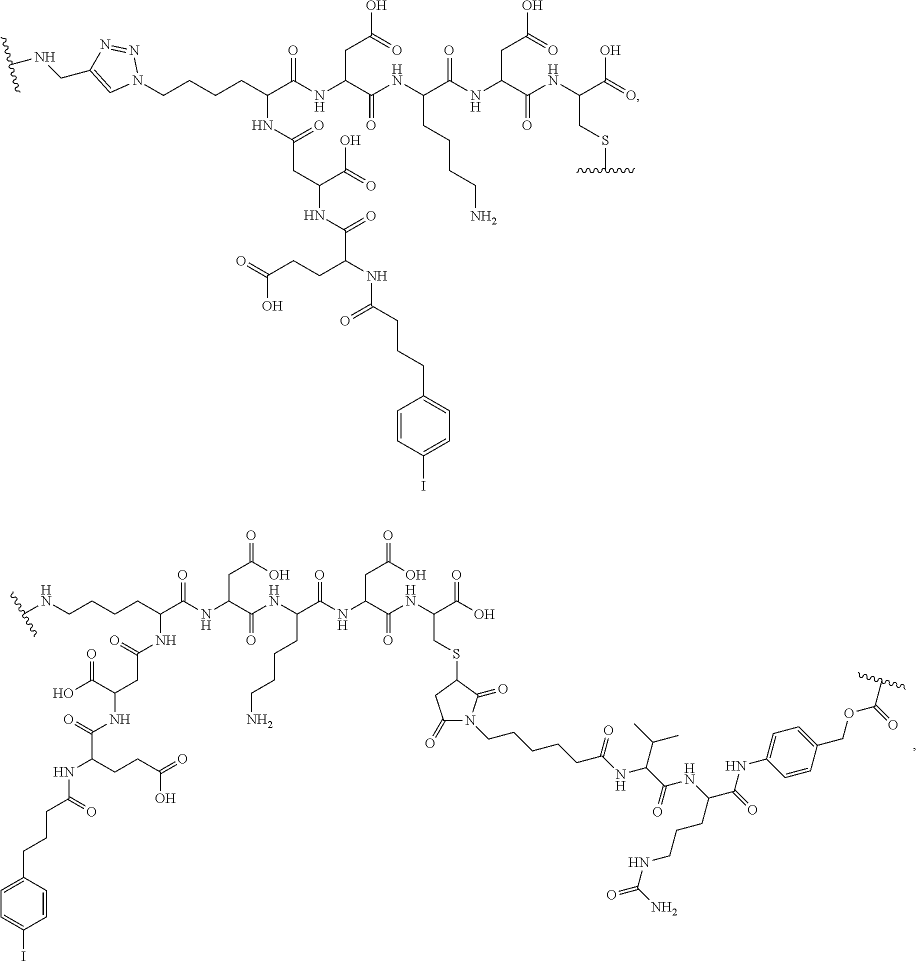 [SMILES: *NCc1cn(CCCCC(NC(=O)CC(NC(=O)C(CCC(=O)O)NC(=O)CCCc2ccc(I)cc2)C(=O)O)C(=O)NC(CC(=O)O)C(=O)NC(CCCCN)C(=O)NC(CC(=O)O)C(=O)NC(CS*)C(=O)O)nn1 ; *NCCCCC(NC(=O)CC(NC(=O)C(CCC(=O)O)NC(=O)CCCc1ccc(I)cc1)C(=O)O)C(=O)NC(CC(=O)O)C(=O)NC(CCCCN)C(=O)NC(CC(=O)O)C(=O)NC(CSC1CC(=O)N(CCCCCC(=O)NC(C(=O)NC(CCCNC(N)=O)C(=O)Nc2ccc(COC(*)=O)cc2)C(C)C)C1=O)C(=O)O]

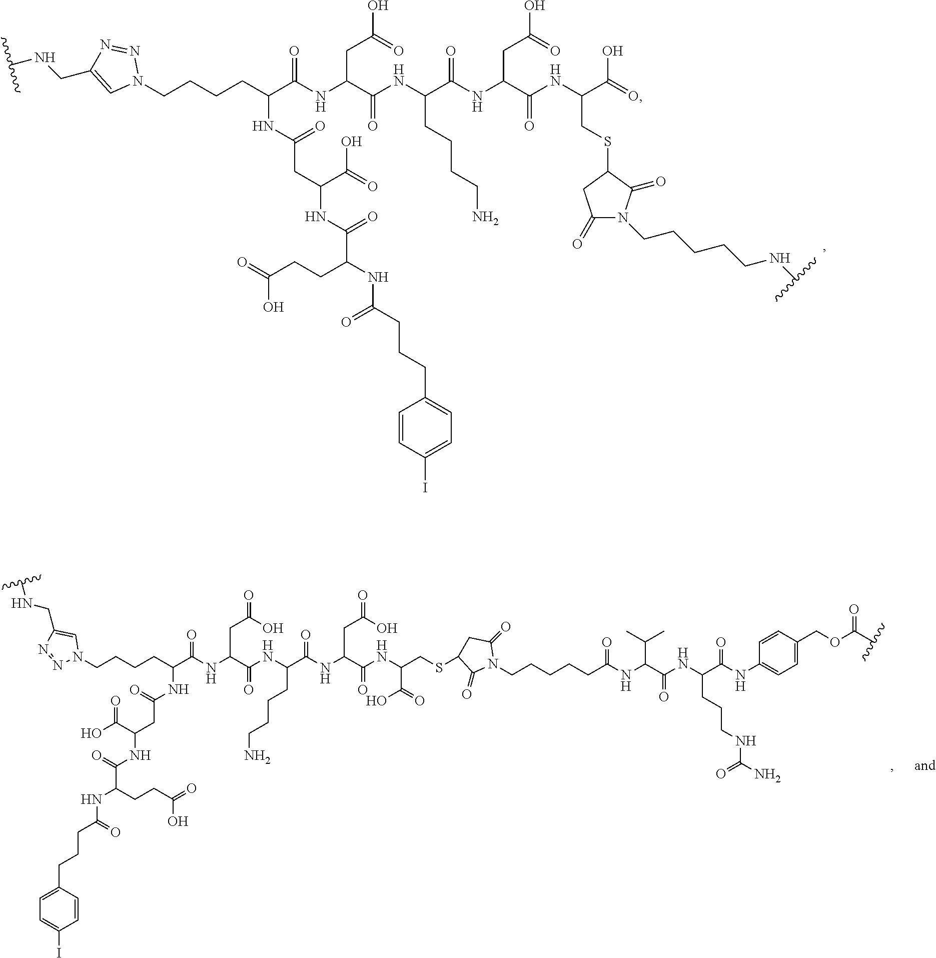 [SMILES: *NCCCCCN1C(=O)CC(SCC(NC(=O)C(CC(=O)O)NC(=O)C(CCCCN)NC(=O)C(CC(=O)O)NC(=O)C(CCCCn2cc(CN*)nn2)NC(=O)CC(NC(=O)C(CCC(=O)O)NC(=O)CCCc2ccc(I)cc2)C(=O)O)C(=O)O)C1=O ; *NCc1cn(CCCCC(NC(=O)CC(NC(=O)C(CCC(=O)O)NC(=O)CCCc2ccc(I)cc2)C(=O)O)C(=O)NC(CC(=O)O)C(=O)NC(CCCCN)C(=O)NC(CC(=O)O)C(=O)NC(CSC2CC(=O)N(CCCCCC(=O)NC(C(=O)NC(CCCNC(N)=O)C(=O)Nc3ccc(COC(*)=O)cc3)C(C)C)C2=O)C(=O)O)nn1]

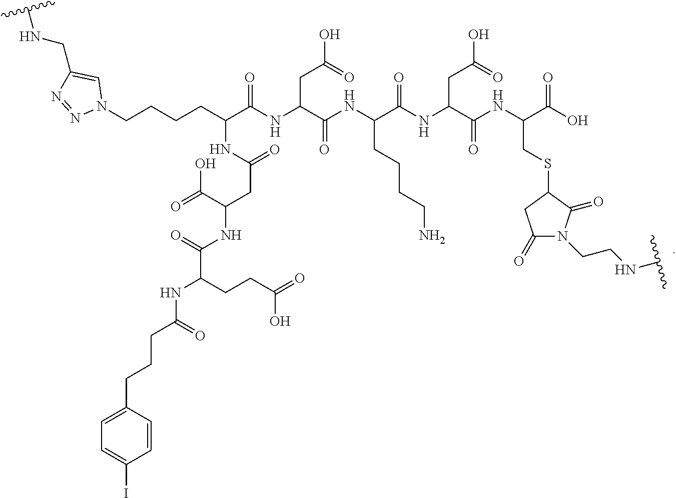 [SMILES: *NCCN1C(=O)CC(SCC(NC(=O)C(CC(=O)O)NC(=O)C(CCCCN)NC(=O)C(CC(=O)O)NC(=O)C(CCCCn2cc(CN*)nn2)NC(=O)CC(NC(=O)C(CCC(=O)O)NC(=O)CCCc2ccc(I)cc2)C(=O)O)C(=O)O)C1=O]

12. The compound according to any one of the preceding embodiments, which has one of the following structures, wherein -D represents —B—C as defined in any one of the preceding embodiments:

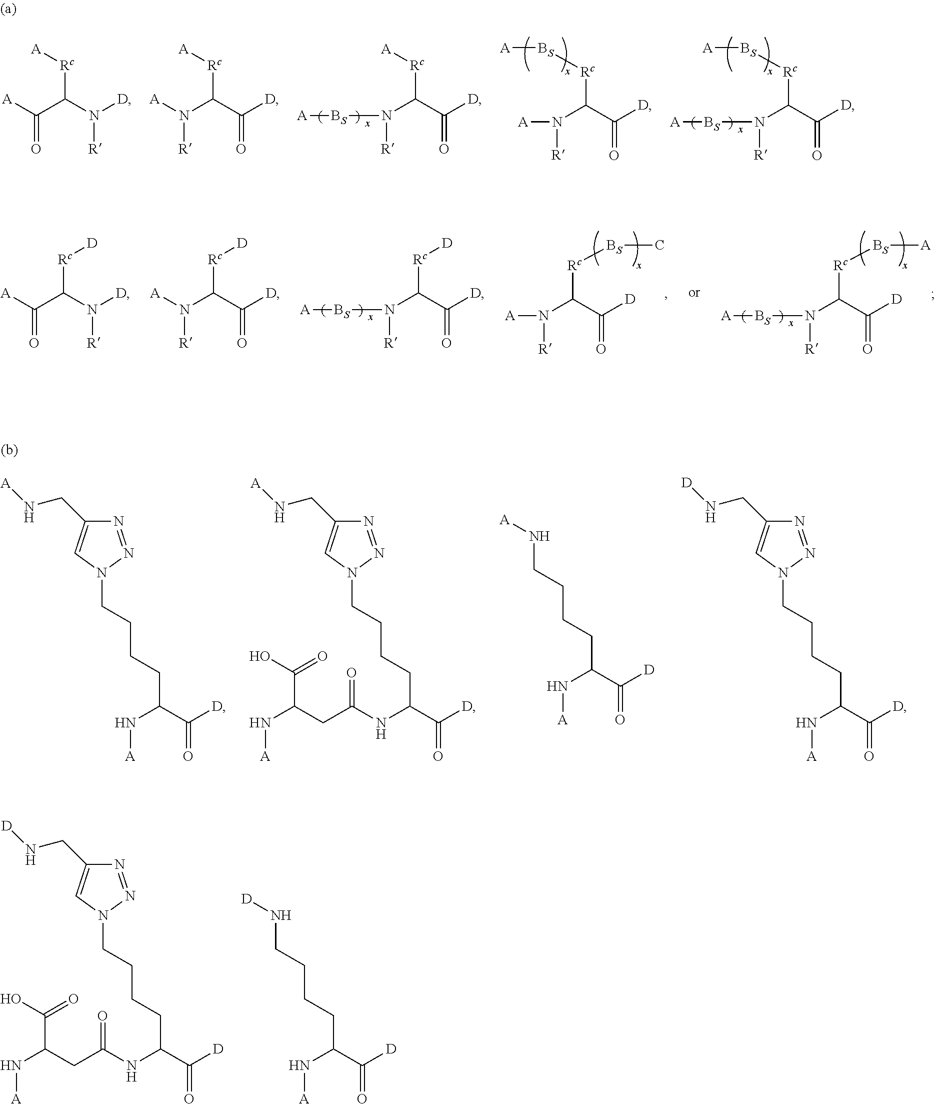

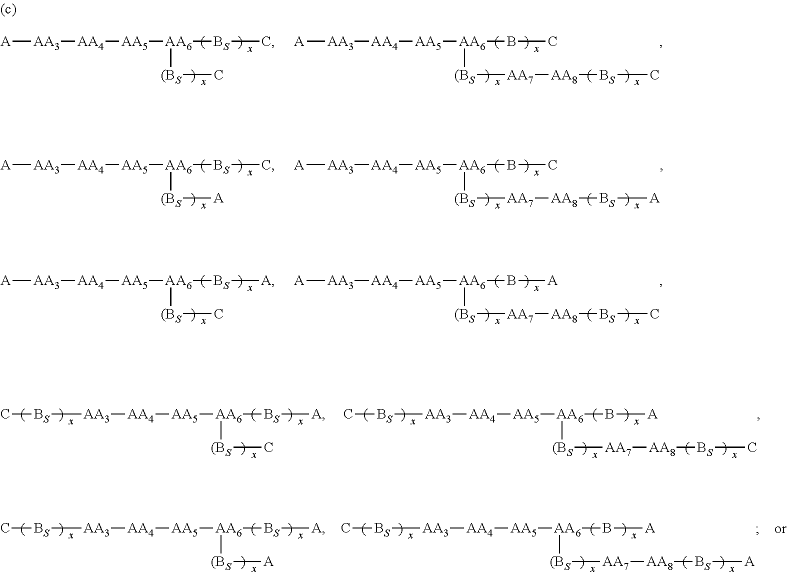

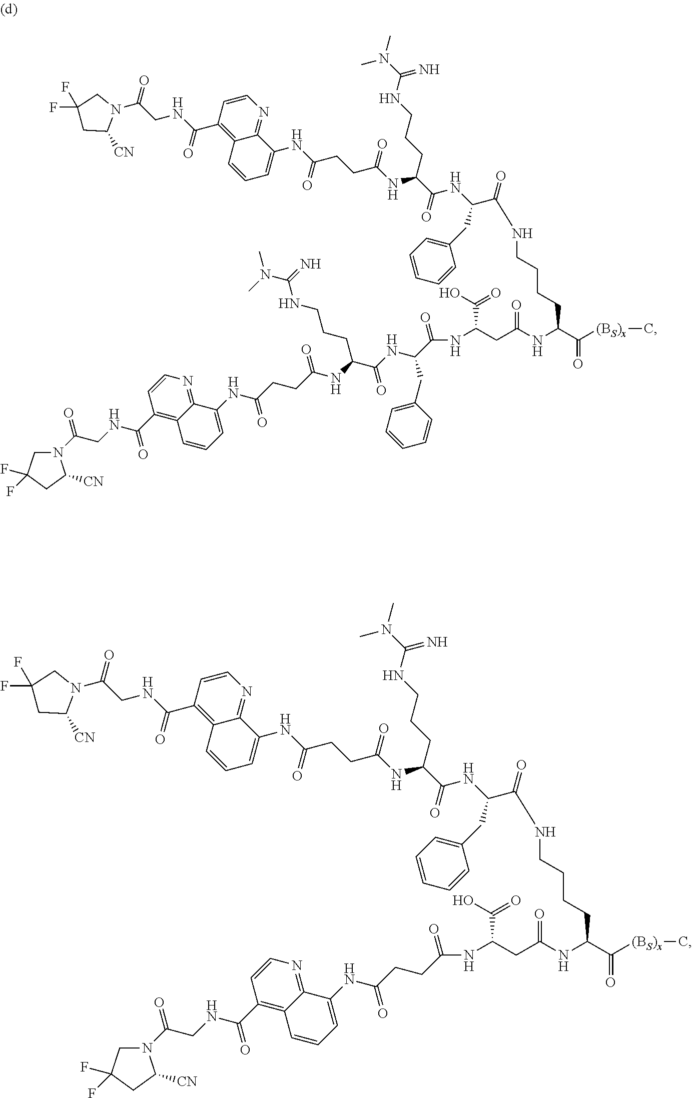

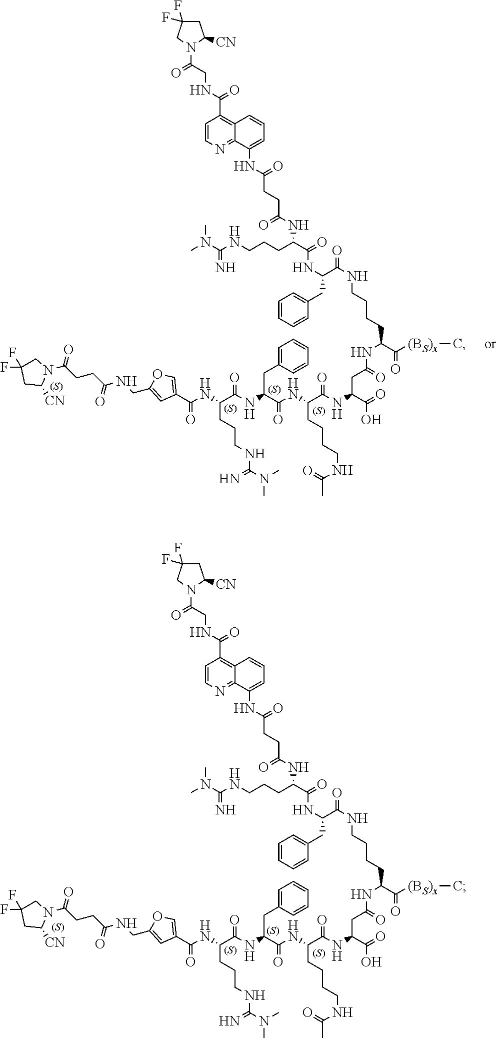

wherein, unless otherwise specified, all groups and variables are defined as in any one of the preceding

13. The compound according to any one of the preceding embodiments, each -D or —B—C is independently represented by any one of the following structures:

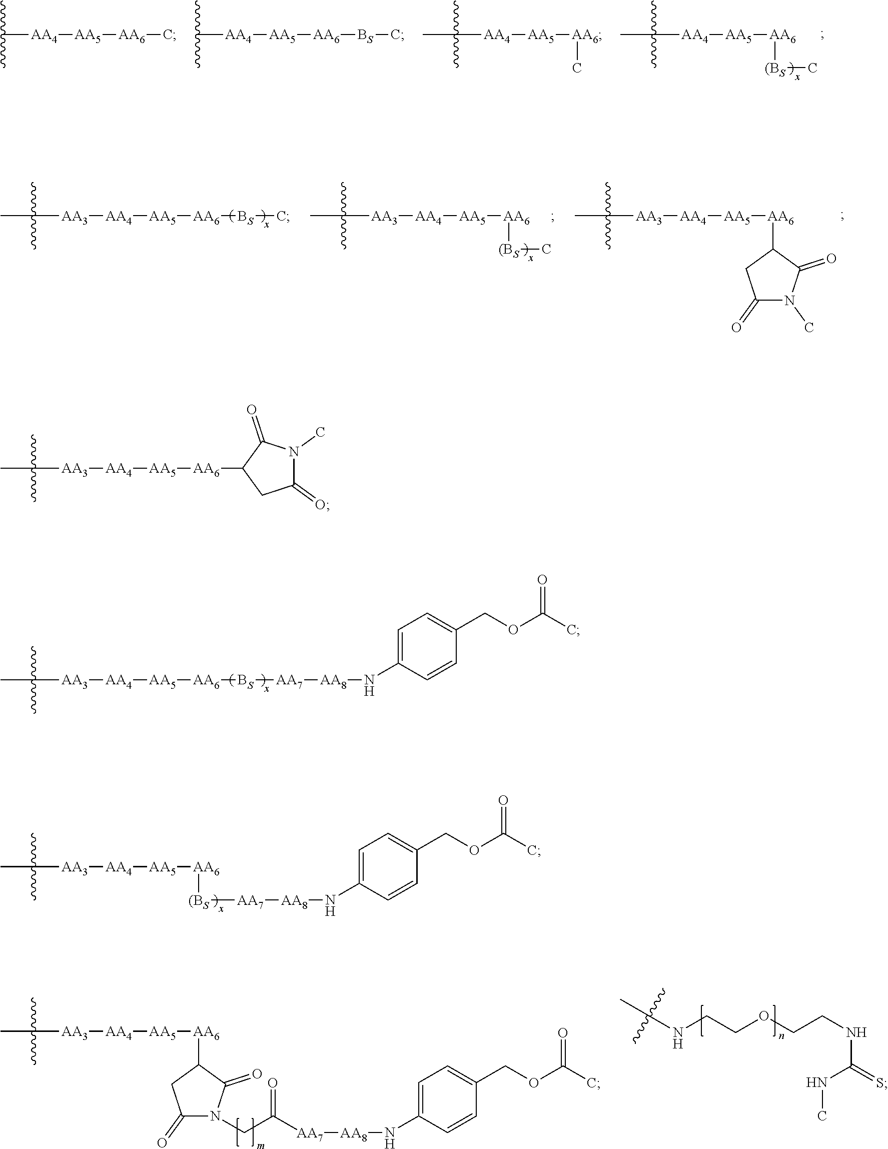

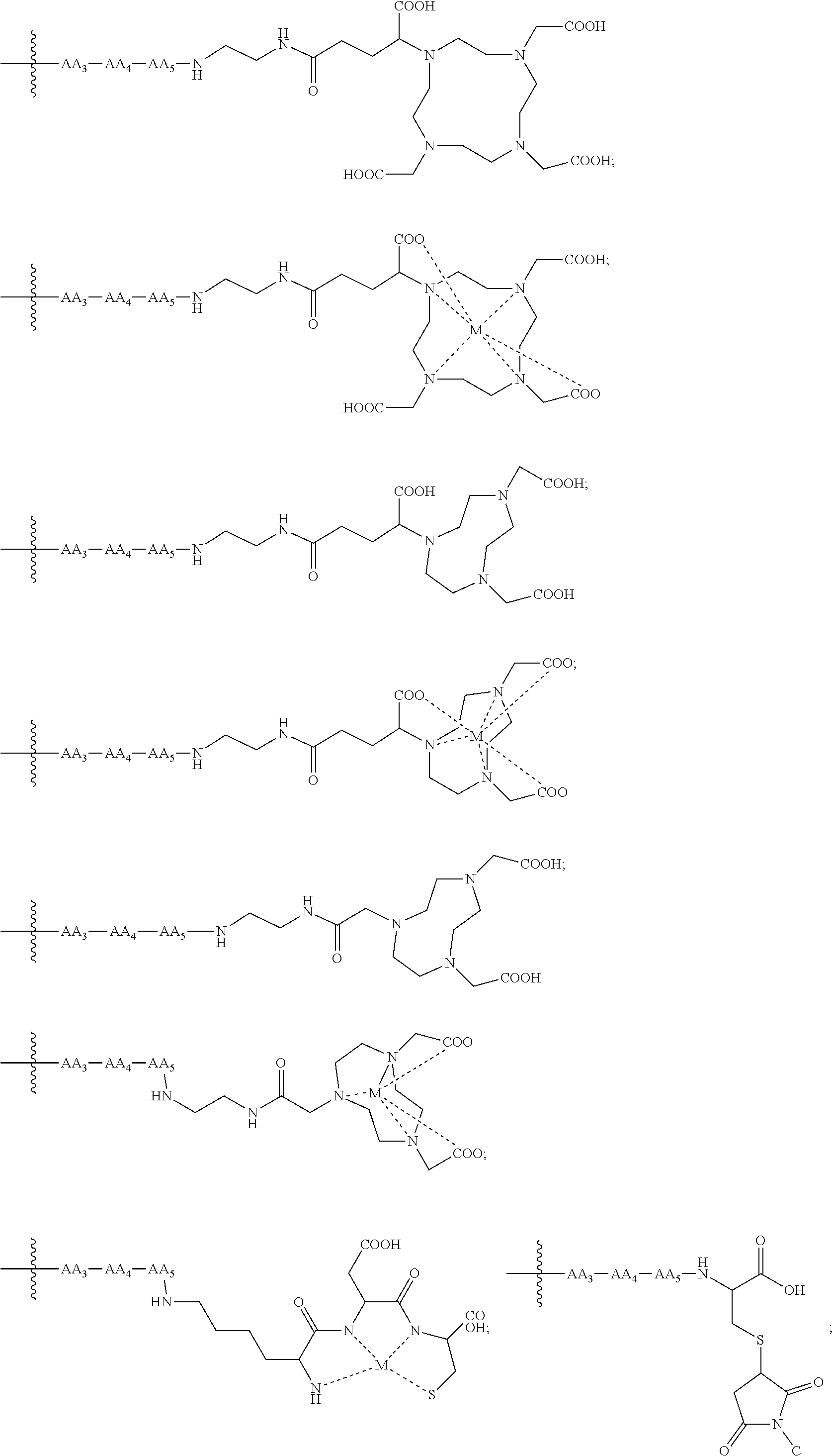

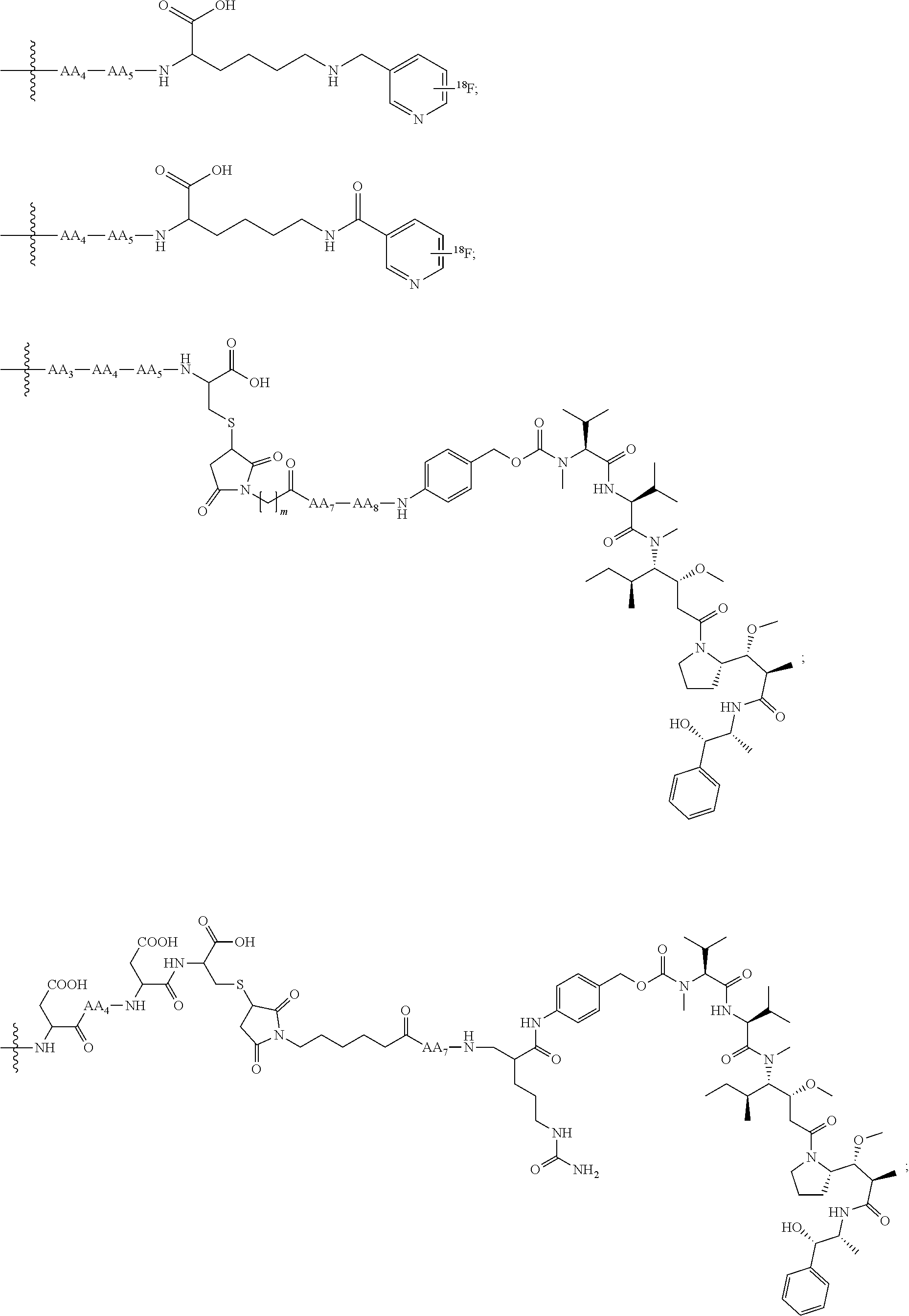

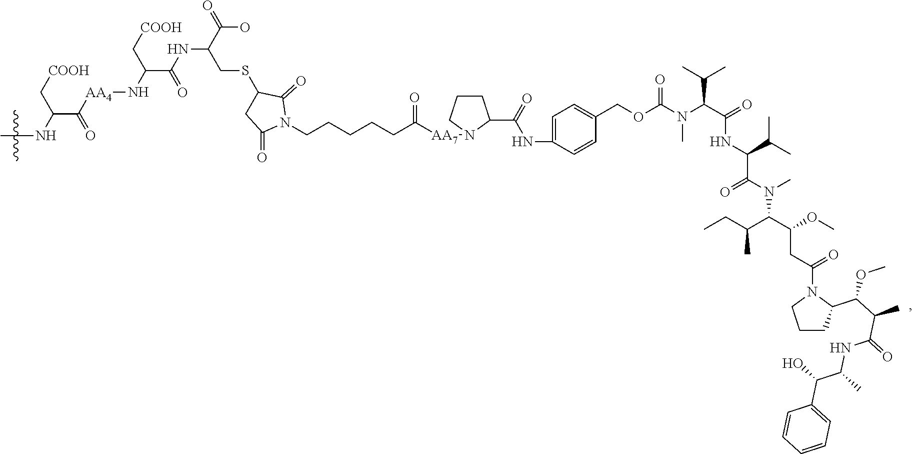

14. The compound according to any one of the preceding embodiments, wherein the moiety C is a chelating agent group suitable for radiolabeling; a radioactive group comprising a radioisotope; a chelate of a radioactive isotope with a chelating agent; a fluorophore group; a cytotoxic and/or cytostatic agent; immunomodulator agent; or
a protein,
wherein preferably:
(a) the chelating agent group suitable for radiolabeling is selected from sulfur colloid, diethylenetriaminepentaacetic acid (DTPA), ethylenediaminetetraacetic acid (EDTA), 1,4,7,10-tetraazacyclododecane-N,N′,N″,N″′-tetraacetic acid (DOTA), 1,4,7-triazacyclononane-N,N′,N″-triacetic acid (NOTA), 1,4,8,11-tetraazacyclotetradecane-N,N′,N″,N″′-tetraacetic acid (TETA), iminodiacetic acid, bis(carboxymethylimidazole)glycine, 6-Hydrazinopyridine-3-carboxylic acid (HYNIC),

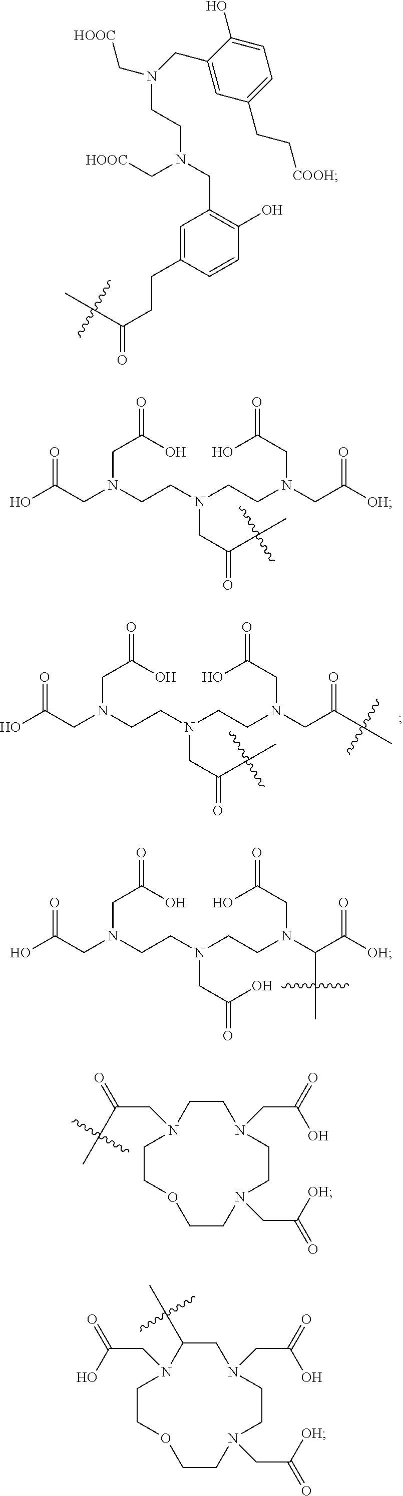

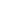

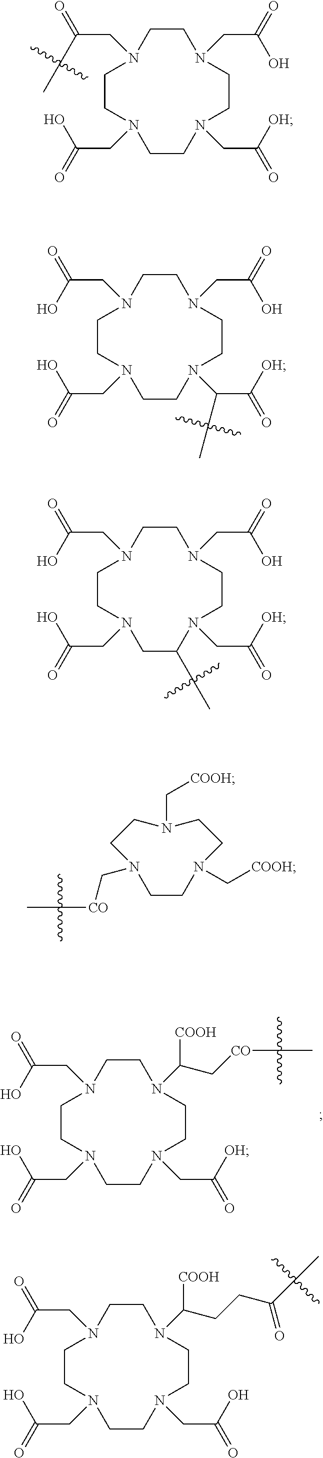 [SMILES: *C(=O)CN1CCN(CC(=O)O)CCN(CC(=O)O)CCN(CC(=O)O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O ; *C(=O)CN1CCN(CC(=O)O)CCN(CC(=O)O)CC1 ; *C(=O)CC(C(=O)O)N1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

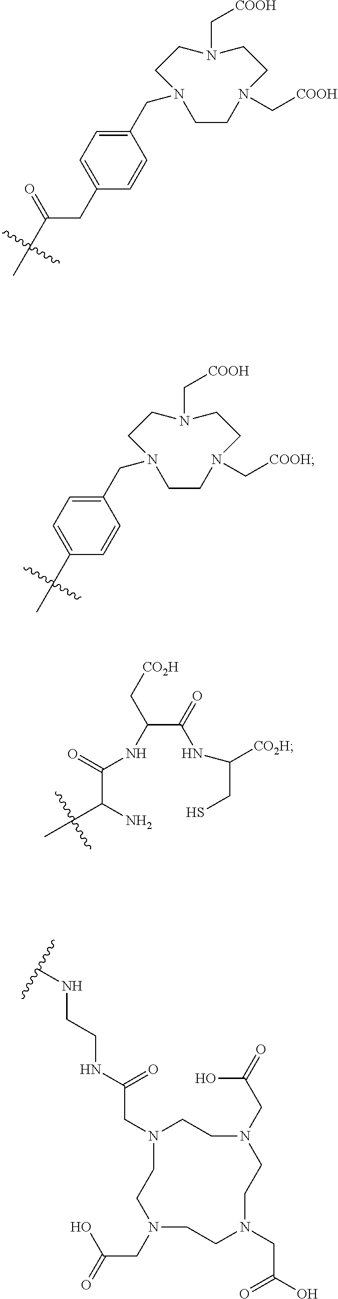 [SMILES: *C(=O)Cc1ccc(CN2CCN(CC(=O)O)CCN(CC(=O)O)CC2)cc1 ; *c1ccc(CN2CCN(CC(=O)O)CCN(CC(=O)O)CC2)cc1 ; *C(N)C(=O)NC(CC(=O)O)C(=O)NC(CS)C(=O)O ; *NCCNC(=O)CN1CCN(CC(=O)O)CCN(CC(=O)O)CCN(CC(=O)O)CC1]

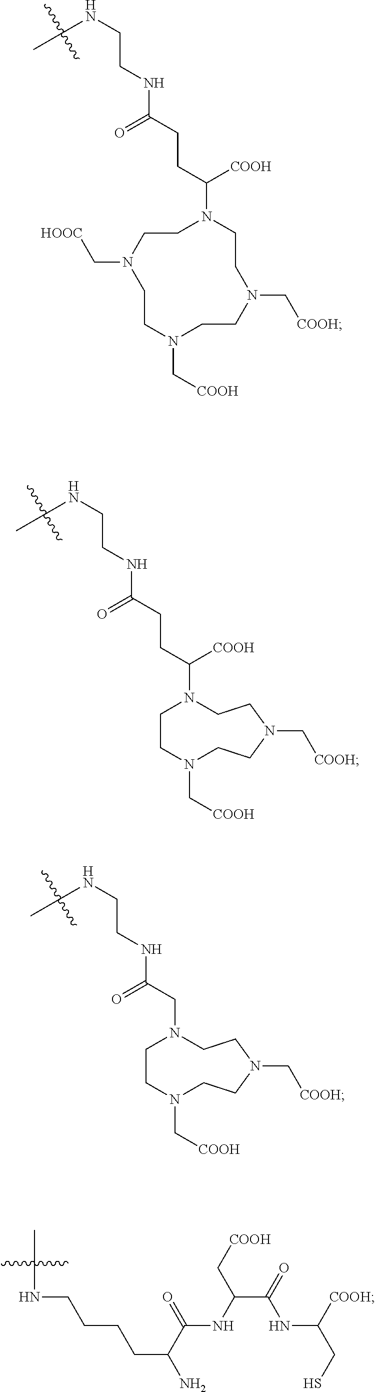 [SMILES: *NCCNC(=O)CCC(C(=O)O)N1CCN(CC(=O)O)CCN(CC(=O)O)CCN(CC(=O)O)CC1 ; *NCCNC(=O)CCC(C(=O)O)N1CCN(CC(=O)O)CCN(CC(=O)O)CC1 ; *NCCNC(=O)CN1CCN(CC(=O)O)CCN(CC(=O)O)CC1 ; *NCCCCC(N)C(=O)NC(CC(=O)O)C(=O)NC(CS)C(=O)O]

or has a structure according to the following formula:

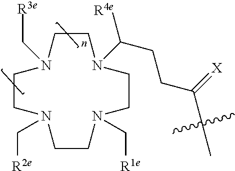

wherein:
n is 0, 1, 2, 3, 4 or 5; preferably 1;
R^{1e} is independently H, COOH, aryl-COOH or heteroaryl-COOH; preferably COOH;
R^{2e} is independently H, COOH, aryl-COOH or heteroaryl-COOH; preferably COOH;
each R^{3e} is independently H, COOH, aryl-COOH or heteroaryl-COOH; preferably COOH;
R^{4e} is independently H, COOH, aryl-COOH or heteroaryl-COOH; preferably COOH; and
X is O, NH or S; preferably O; or
has a structure according to the following formula:

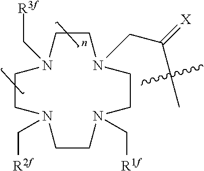

wherein:
n is 0, 1, 2, 3, 4 or 5; preferably 1
R^{1f} is independently H, COOH, aryl-COOH or heteroaryl-COOH; preferably COOH;
R^{2f} is independently H, COOH, aryl-COOH or heteroaryl-COOH; preferably COOH;
R^{3f} is independently H, COOH, aryl-COOH or heteroaryl-COOH; preferably COOH; and
X is O, NH or S; preferably O;
(b) the radioactive group comprising a radioisotope is selected from ²²³Ra, ⁸⁹Sr, ^{94m}Tc, ^{99m}Tc, ¹⁸⁶Re, ¹⁸⁸Re, ²⁰³Pb, ⁶⁷Ga, ⁶⁸Ga, ⁴⁷Sc, ¹¹¹I, ⁹⁷Ru, ⁶²Cu, ⁶⁴Cu, ⁸⁶Y, ⁸⁸Y, ⁹⁰Y, ¹²¹Sn, ¹⁶¹Th, ¹⁵³Sm, ¹⁶⁶Ho, ¹⁰⁵Rh, ¹⁷⁷Lu, ¹²³I, ¹²⁴I, ¹²⁵I, ¹³¹I, ¹⁸F, ²¹¹At, ²²⁵Ac, ⁸⁹Sr, ²²⁵Ac, ^{117m}Sn and ¹⁶⁹Er;
(c) the chelate of a radioactive isotope is a chelate of an isotope listed under (b) above and/or with a chelating agent listed under (a) above; or moiety C is a group selected from any of the following structures:

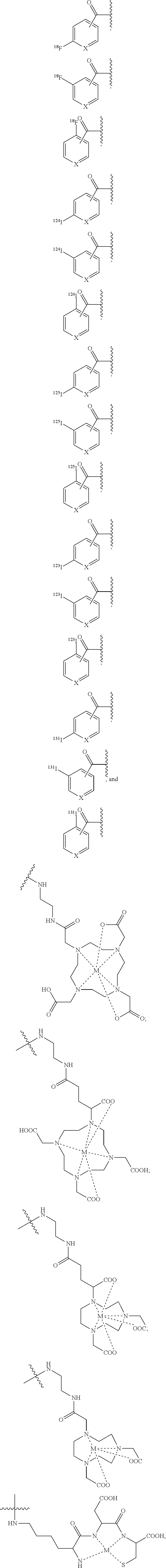

wherein M is a radioactive isotope, preferably selected among the list under (b) above; more preferably:

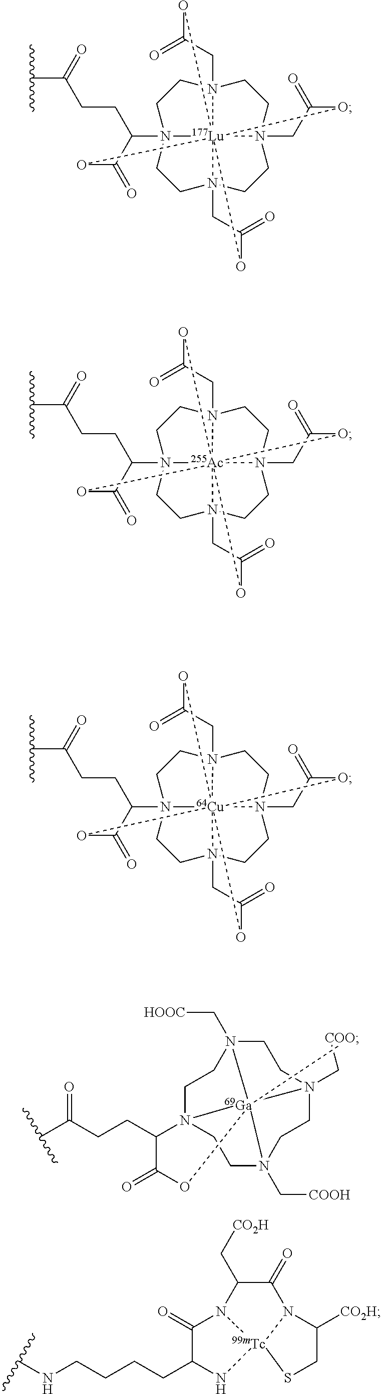

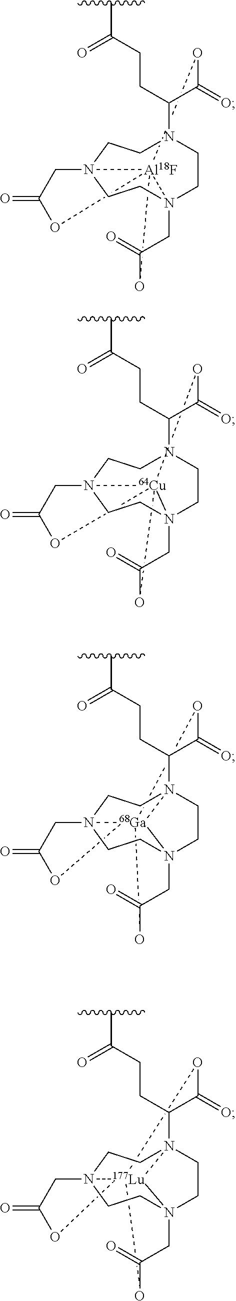

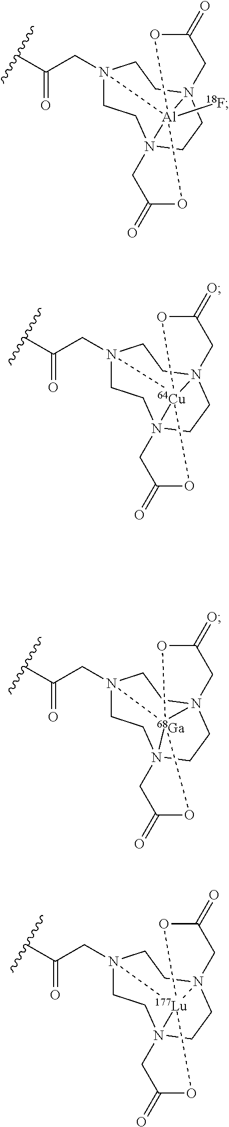

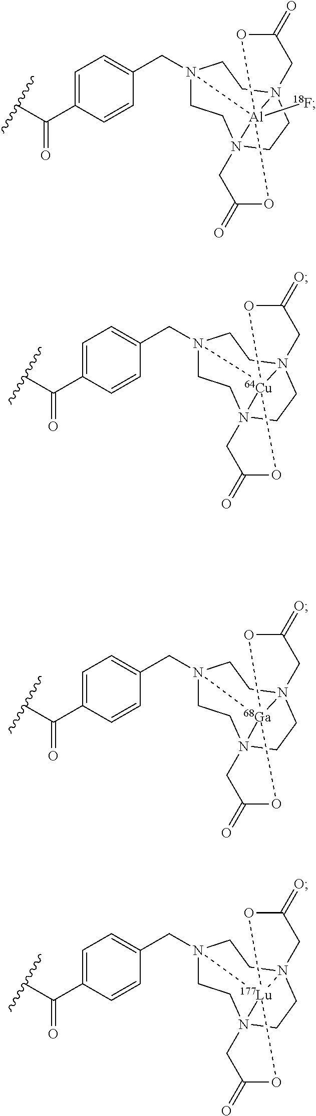

(d) the fluorophore group is selected from a xanthene dye, acridine dye, oxazine dye, cyanine dye, styryl dye, coumarine dye, porphine dye, fluorescent metal-ligand-complex, fluorescent protein, nanocrystals, perylene dye, boron-dipyrromethene dye and phtalocyanine dye, preferably selected from the following structures:

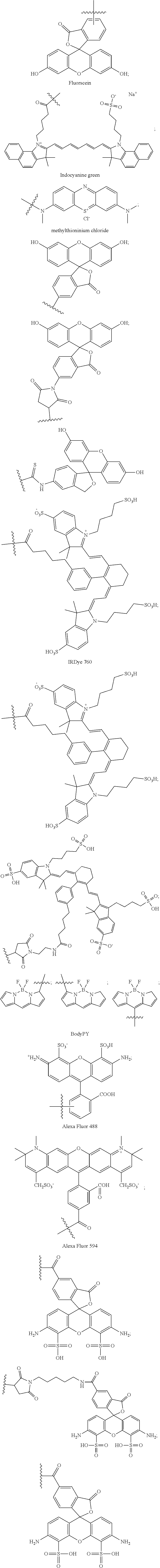

(e) the cytotoxic and/or cytostatic agent is selected from chemotherapeutic agent selected from the group consisting of topoisomerase inhibitors, alkylating agents, antimetabolites, antibiotics, mitotic disrupters, DNA intercalating agents, DNA synthesis inhibitors, DNA-RNA transcription regulator, enzyme inhibitors, gene regulators, hormone response modifiers, hypoxia-selective cytotoxins, epidermal growth factor inhibitors, anti-vascular agents and a combination of two or more thereof, preferably selected from the following structures:

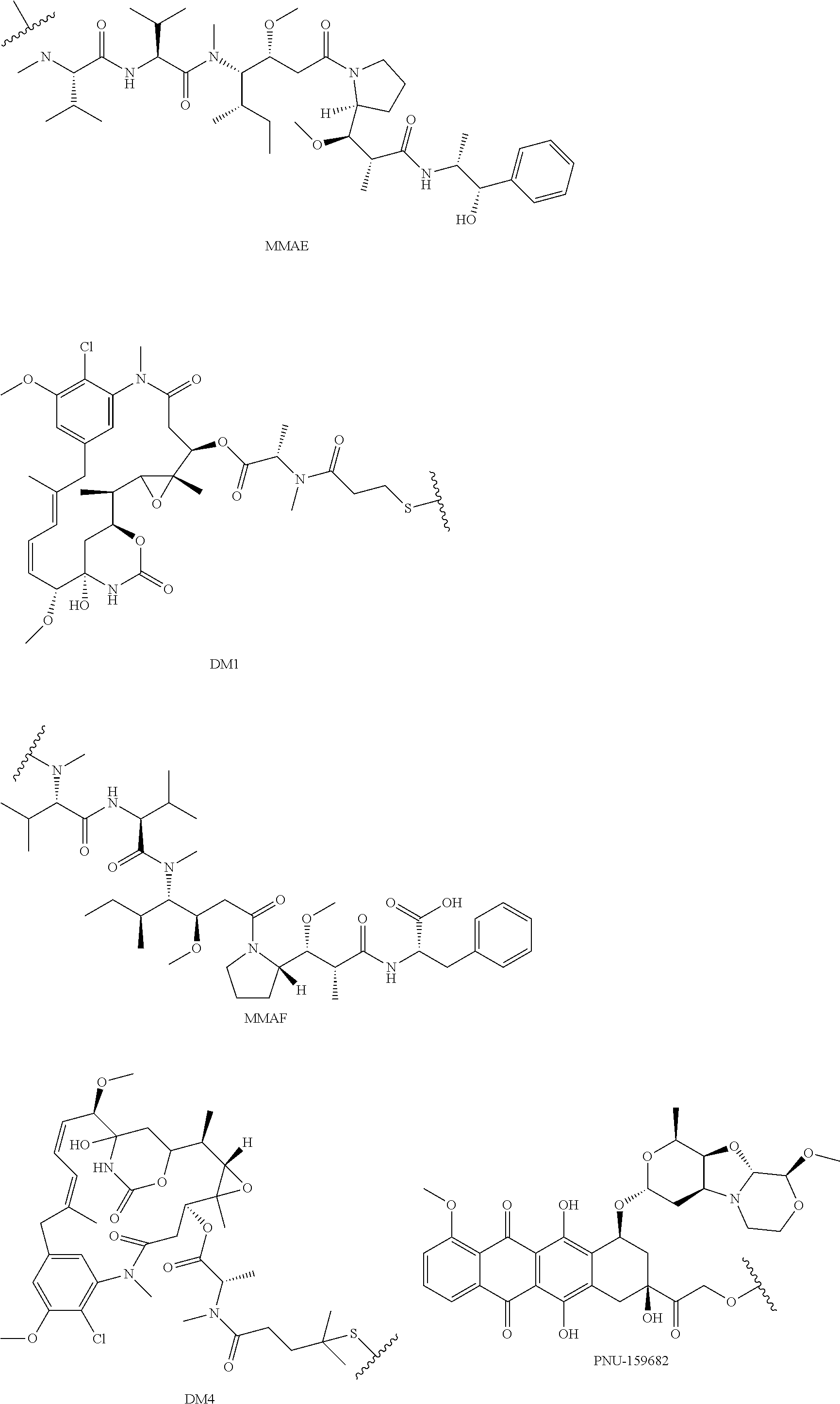

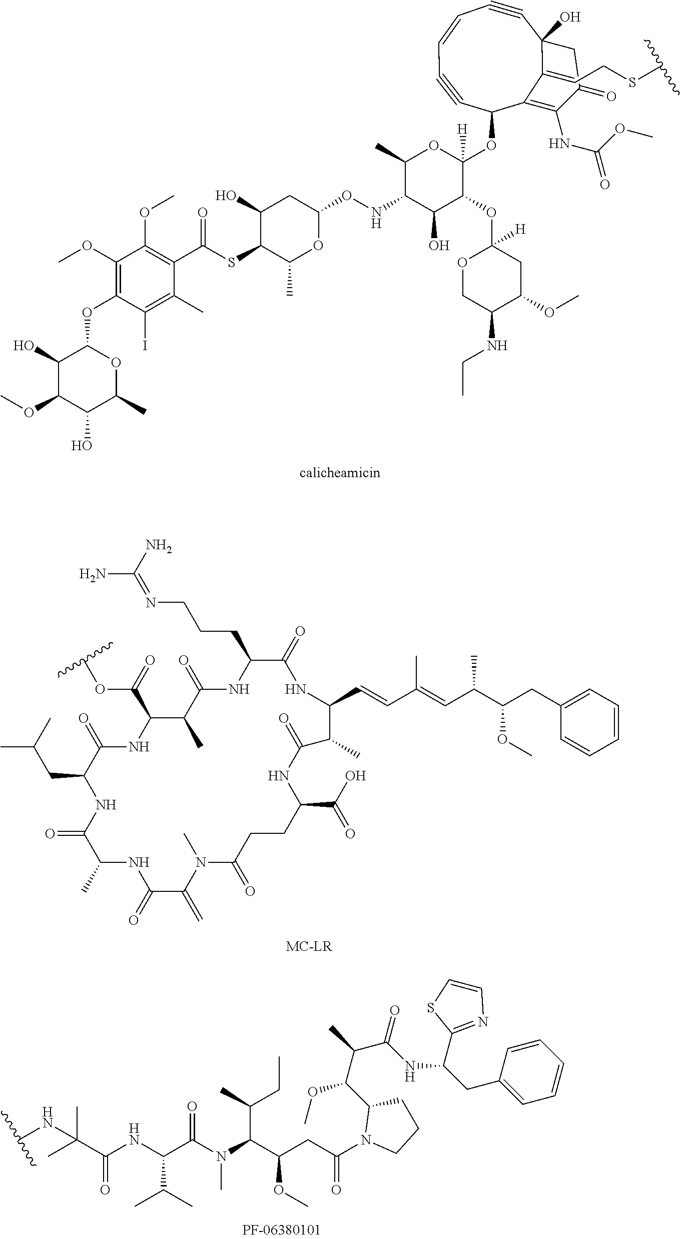

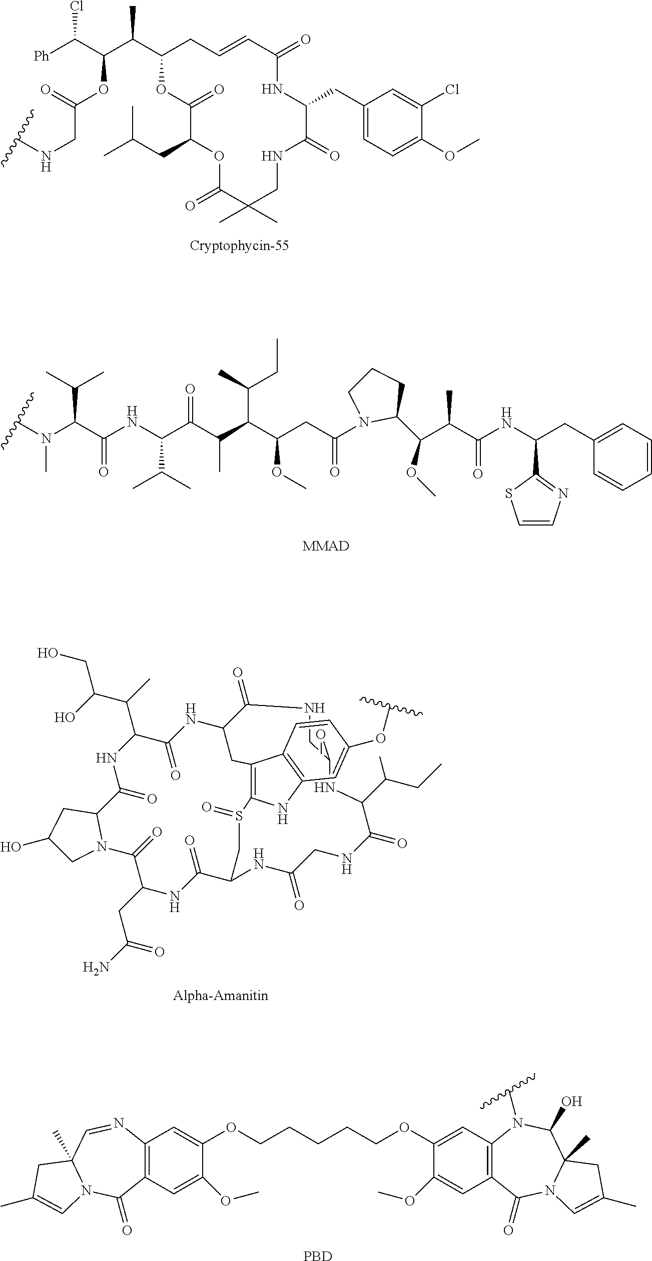

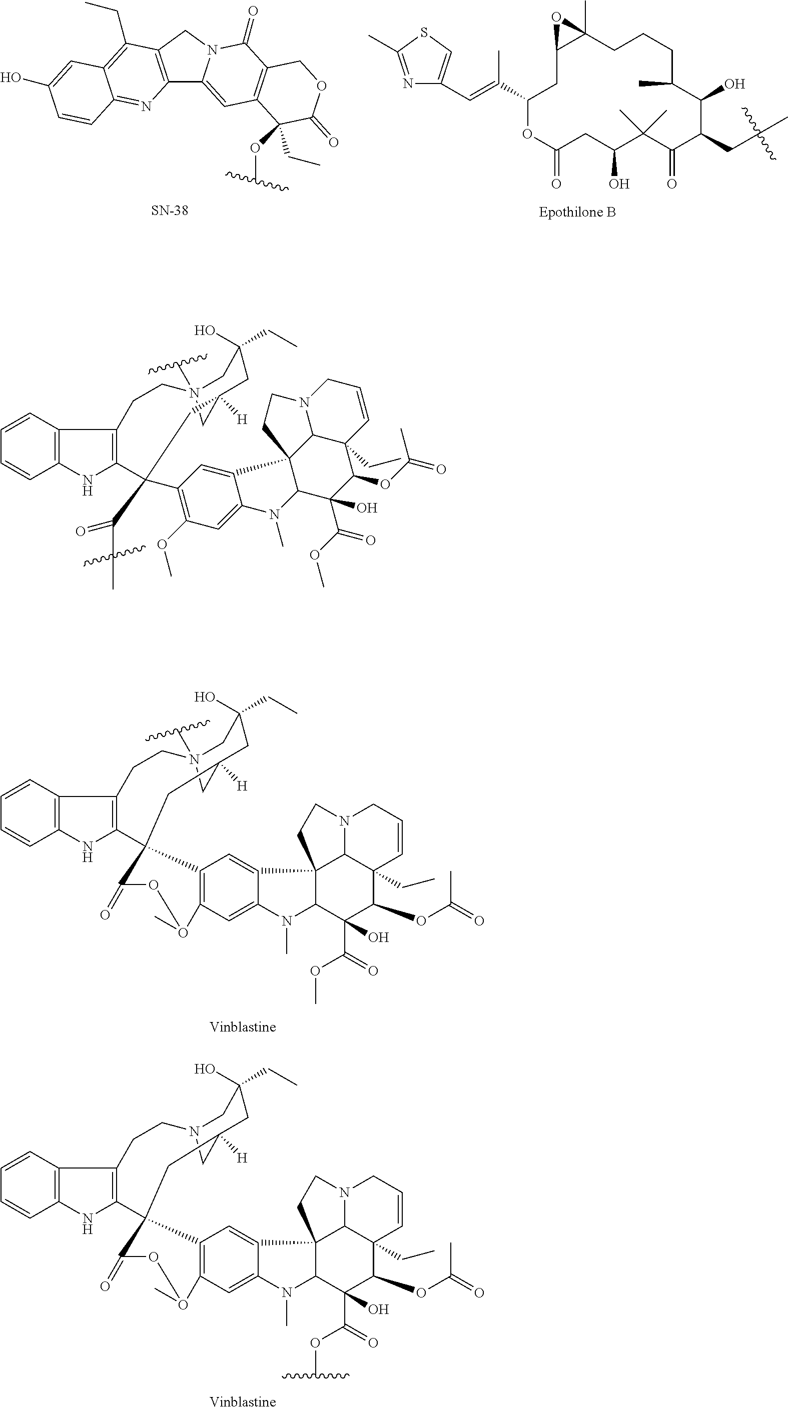

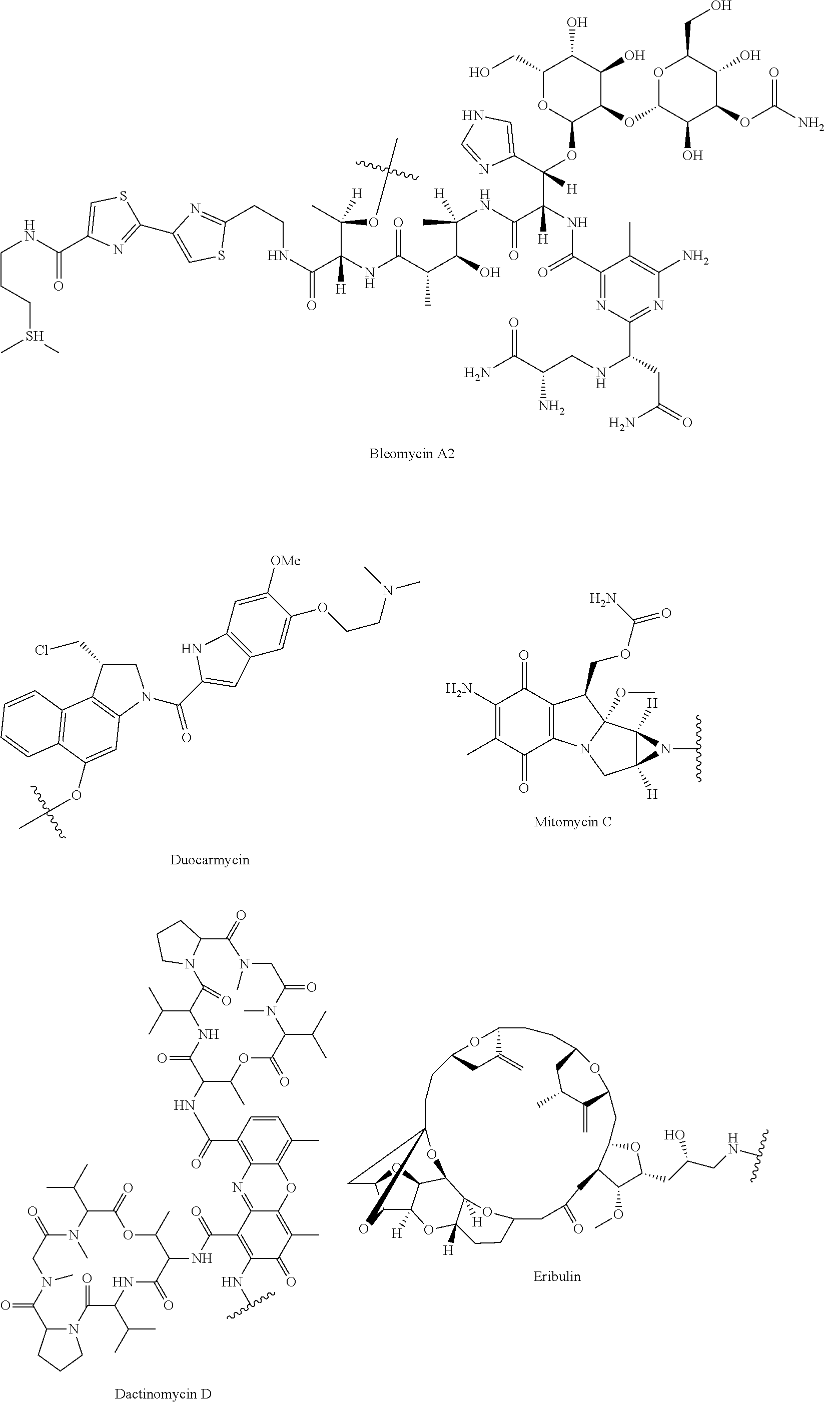

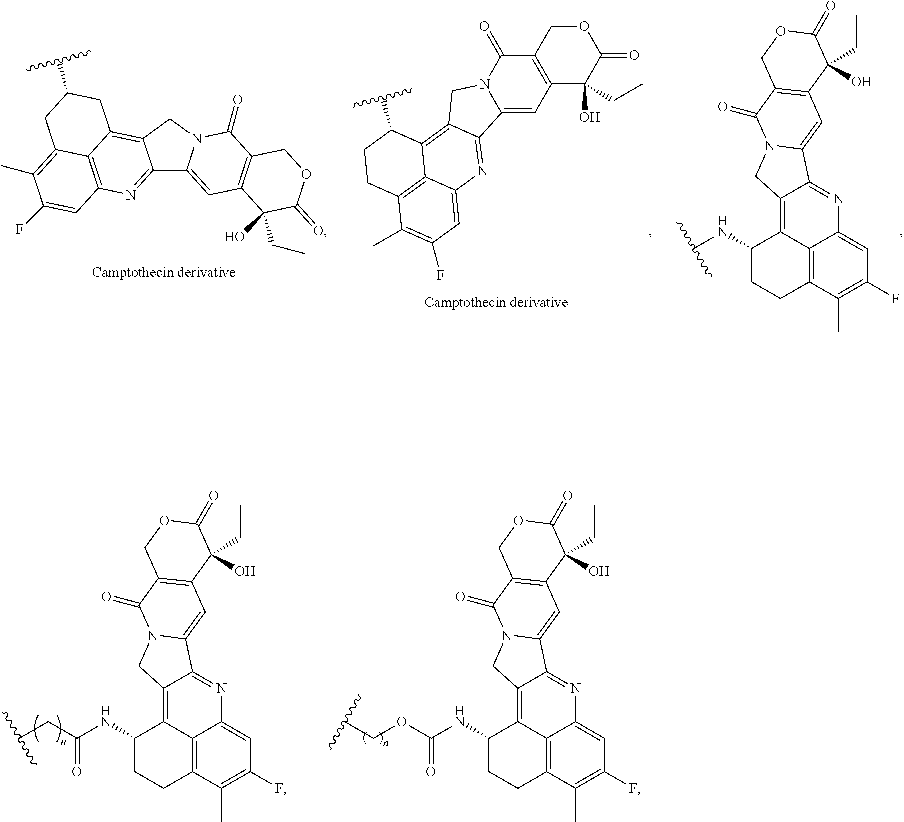

wherein each n is 0, 1, 2, 3, 4, 5 or 6;

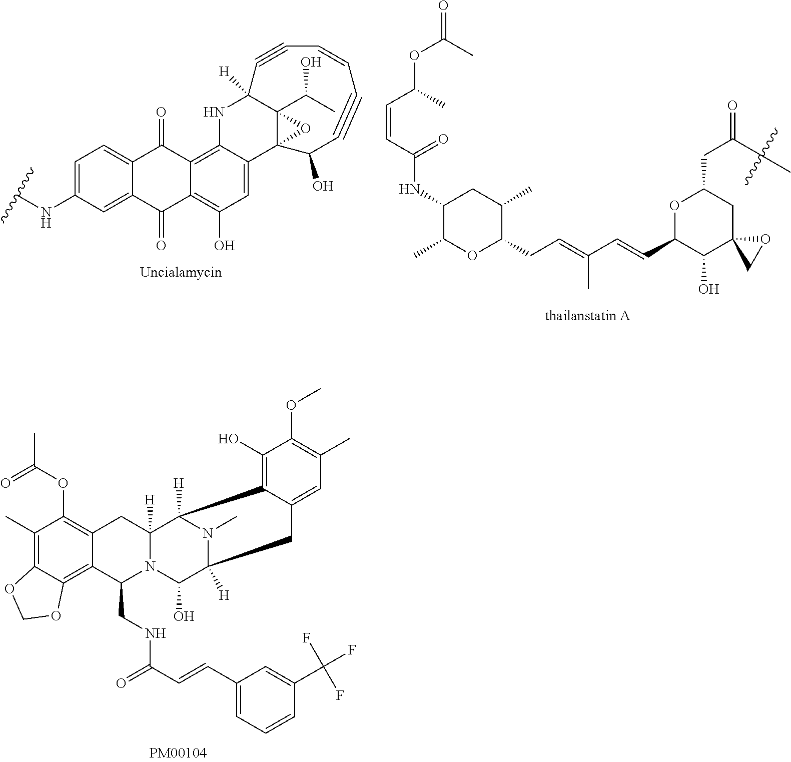

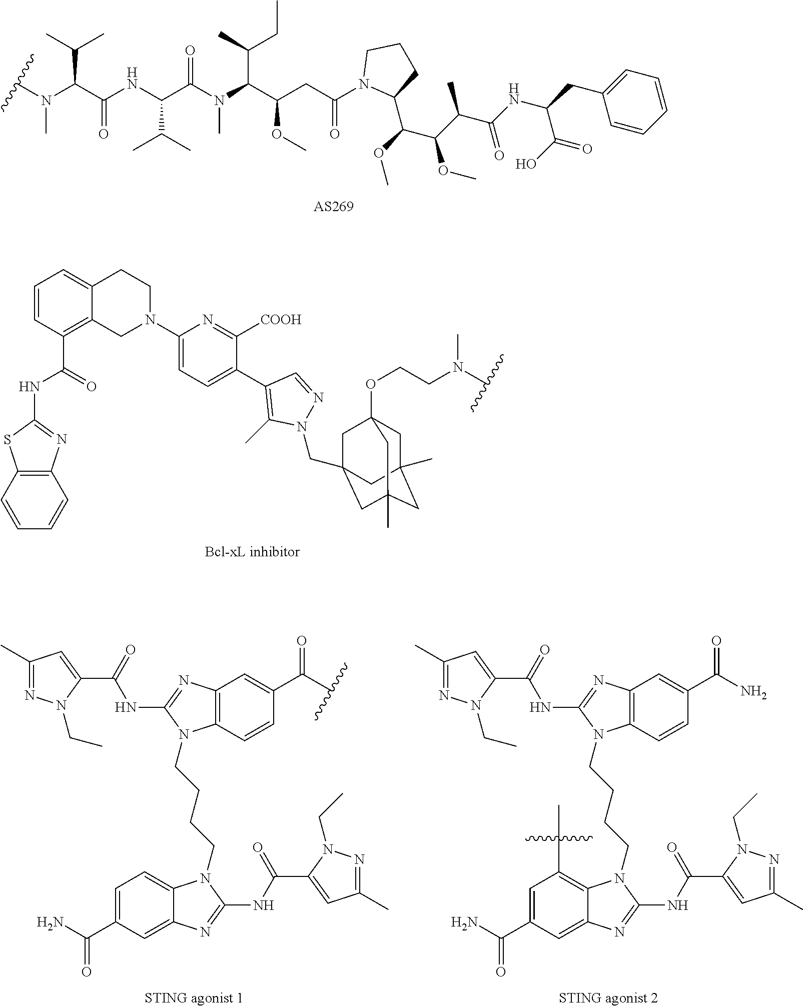

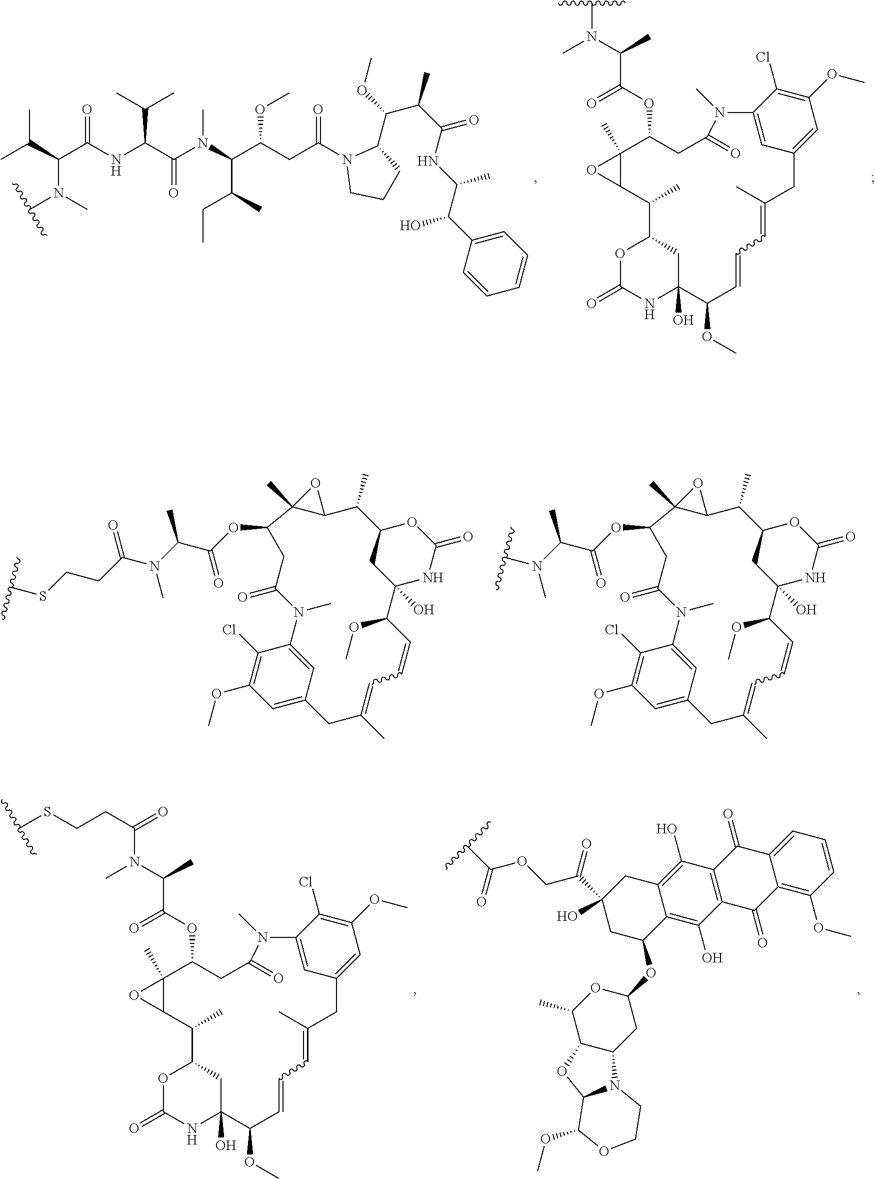

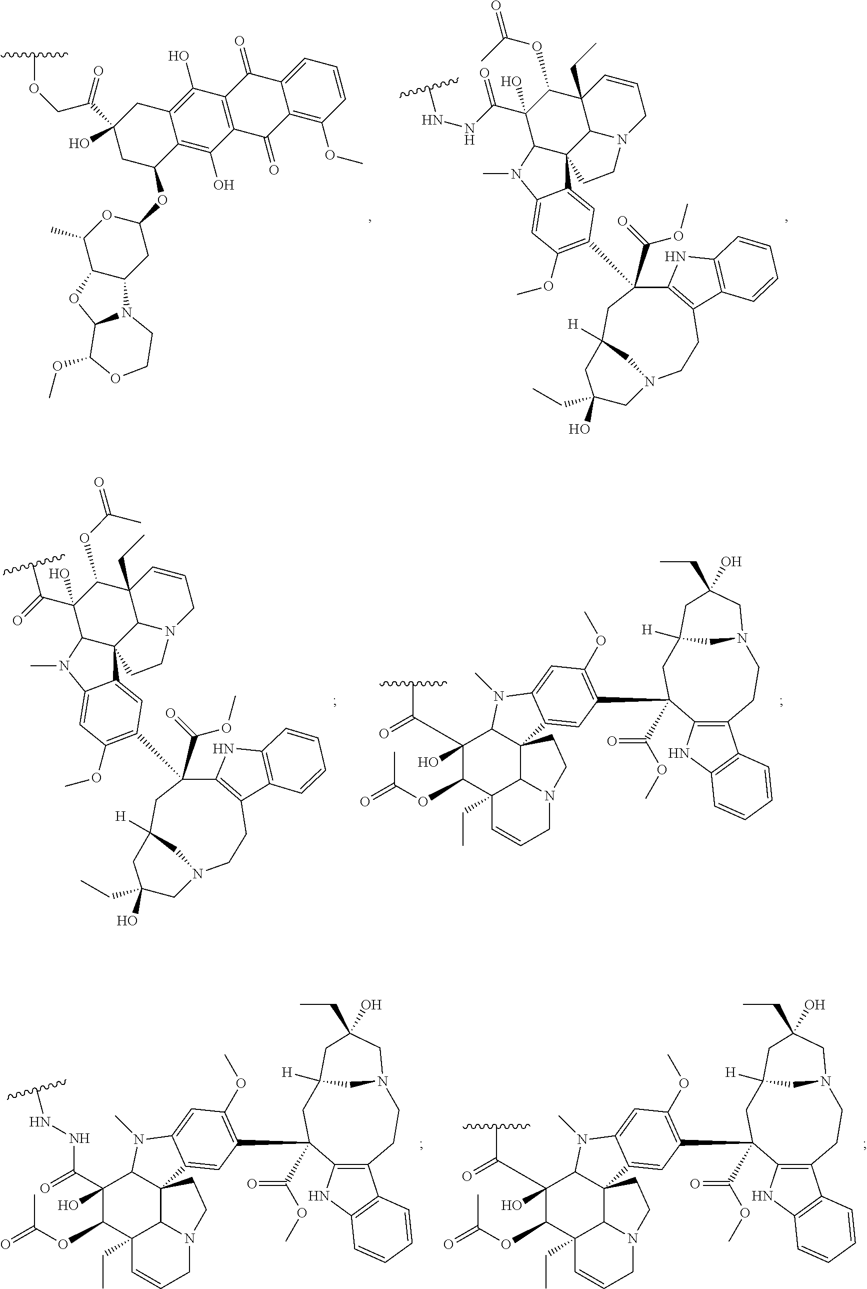

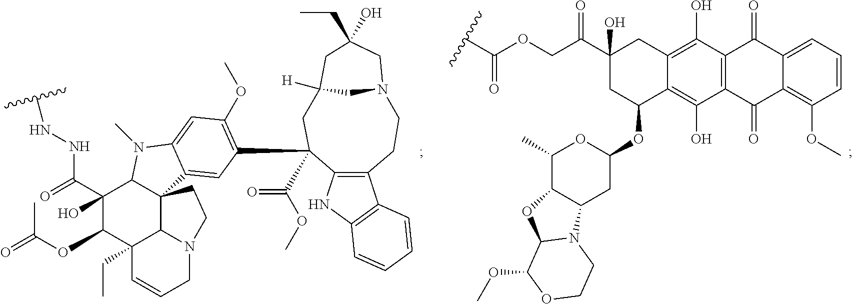

moiety C is an auristatin, preferably having a structure according to the following formula:

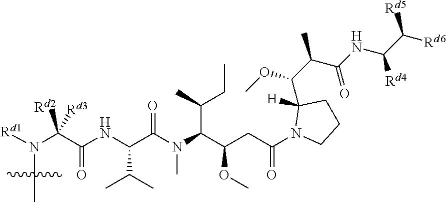

wherein:
R^{1d} is independently H or C₁-C₆ alkyl; preferably H or CH₃;
R^{2d} is independently C₁-C₆ alkyl; preferably CH₃ or iPr;
R^{3d} is independently H or C₁-C₆ alkyl; preferably H or CH₃;
R^{4d} is independently H, C₁-C₆ alkyl, COO(C₁-C₆ alkyl), CON(H or C₁-C₆ alkyl), C₃-C₁₀ aryl or C3-C10 heteroaryl; preferably H, CH₃, COOH, COOCH₃ or thiazolyl;
R^{5d} is independently H, OH, C₁-C₆ alkyl; preferably H or OH; and
R^{6d} is independently C₃-C₁₀ aryl or C₃-C₁₀ heteroaryl; preferably optionally substituted phenyl or pyridyl, wherein preferably, moiety C is derived from MMAE or MMAF;
(f) the immunomodulator agent is selected from molecules known to be able to modulate the immune system, such as ligands of CD3, CD25, TLRs, STING, 4-1BBL, 4-1BB, PD-1, mTor, PDL-1, NKG-2D IMiDs, wherein ligands can be agonists and/or antagonist; or
(g) the protein is selected from cytokines, such as IL2, IL10, IL12, IL15, TNF, Interferon Gamma, or is an antibody.

15. A compound having a structure selected from the conjugates listed in Table 2 or 4, its individual diastereoisomers, its hydrates, its solvates, its crystal forms, its individual tautomers or a pharmaceutically acceptable salt thereof:

16. A pharmaceutical composition comprising the compound according to any one of the preceding embodiments, and a pharmaceutically acceptable excipient.

17. The compound or the pharmaceutical composition according to any one of the preceding embodiments for use in:
(a) a method for treatment of the human or animal body by surgery or therapy or a diagnostic method practised on the human or animal body; or
(b) a method for therapy or prophylaxis of a subject suffering from or having risk for a disease or disorder; or
(c) a method for guided surgery practised on a subject suffering from or having risk for a disease or disorder; or
(d) a method for diagnosis of a disease or disorder, the method being practised on the human or animal body and involving a nuclear medicine imaging technique, such as Positron Emission Tomography (PET) or Single Photon Emission Computed Tomography (SPECT); or
(e) a method for targeted delivery of a therapeutic or diagnostic agent to a subject suffering from or having risk for a disease or disorder,
   wherein in each of the preceding (b)-(e), said disease or disorder is independently selected from cancer, inflammation, atherosclerosis, fibrosis, tissue remodeling and keloid disorder, preferably wherein the cancer is selected from the group consisting of breast cancer, pancreatic cancer, small intestine cancer, colon cancer, multi-drug resistant colon cancer, rectal cancer, colorectal cancer, metastatic colorectal cancer, lung cancer, non-small cell lung cancer, head and neck cancer, ovarian cancer, hepatocellular cancer, oesophageal cancer, hypopharynx cancer, nasopharynx cancer, larynx cancer, myeloma cells, bladder cancer, cholangiocarcinoma, clear cell renal carcinoma, neuroendocrine tumor, oncogenic osteomalacia, sarcoma, CUP (carcinoma of unknown primary), thymus cancer, desmoid tumors, glioma, astrocytoma, cervix cancer, skin cancer, kidney cancer and prostate cancer; and wherein in each of the preceding uses or methods, the compound preferably has a prolonged residence at the disease site at a therapeutically or diagnostically relevant level, preferably beyond 1 h, more preferably beyond 6 h post injection.

18. A compound, its individual diastereoisomers, its hydrates, its solvates, its crystal forms, its individual tautomers or a salt thereof, wherein the compound has the structure

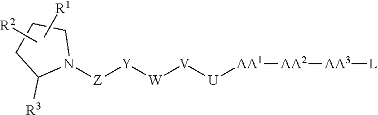

wherein L represents a reactive moiety capable of reacting with a conjugation partner and forming a covalent bond, preferably an amide, ester, carbamate, hydrazone, thiazolidine, methylene alkoxy carbamate, disulphide, alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, aminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide or tetrapeptide linking group; and all other groups as defined as in any of the preceding embodiments.

19. The compound of embodiment 19, wherein:
L is selected from: H, OH, NH₂, N₃, COOH, SH, Hal,

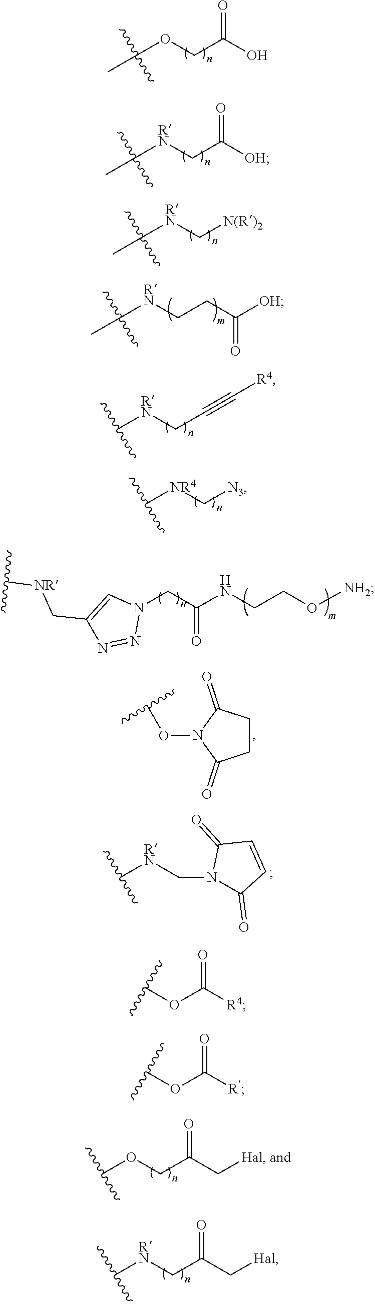

wherein each n is, independently, 0, 1, 2, 3, 4, 5, 6, 7, 8, 9 or 10;
each m is, independently, 0, 1, 2, 3, 4 or 5;
each Hal is F, Cl, Br or I; and
each R⁴ is, independently selected from H, carboxy, alkyl, cycloalkyl, aryl and heteroaryl, wherein each of the foregoing is substituted or unsubstituted, halogen, and cyano;
and/or
wherein AA₃ is absent or is Lys; preferably Lys.

1. A compound, its individual diastereoisomers, its hydrates, its solvates, its crystal forms, its individual tautomers or a pharmaceutically acceptable salt thereof, wherein the compound structure comprises at least one moiety A independently represented by the following structure: 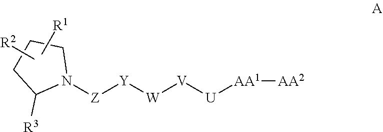
wherein AA¹ has the following structure: 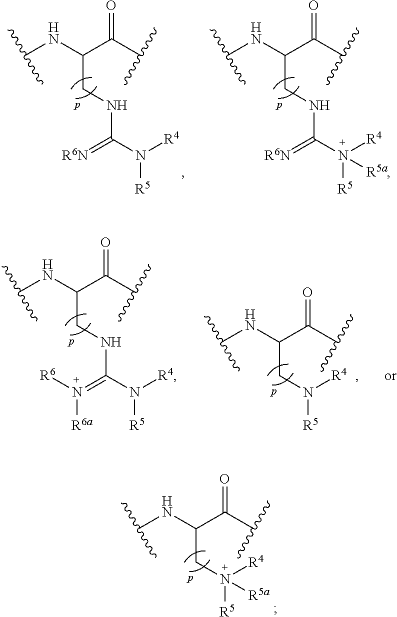
each of R¹ and R² is independently selected from the group consisting of H, OH, halogen, F, C₁₋₆alkyl, —O—C₁₋₆alkyl, —S—C₁₋₆alkyl, and N₃;
R³ is independently selected from: H, —CN, —B(OH)₂, —C(O)alkyl, —C(O)haloalkyl, —C(O)aryl-, —C═C—C(O)aryl, —C═C—S(O)₂aryl, —CO₂H, —SO₃H, —SO₂NH₂, —PO₃H₂, and 5-tetrazolyl;
each of R⁴, R⁵, R^{5a}, R⁶, and R^{6a} is independently H, OH, optionally substituted C₁-C₆ alkyl, optionally substituted C₁-C₆ haloalkyl, optionally substituted —C(O)C₁-C₅ alkyl, or optionally substituted —C(O)C₁-C₅ haloalkyl, and wherein two of them can be joined together or any one of them can be joined to the (CH₂)ₚ moiety to form a ring structure; or wherein R⁴, R⁵, R^{5a}, R⁶, and R^{6a} is independently any of the preceding groups and at least one of R⁴, R⁵, R^{5a}, R⁶, and R^{6a} is not H;
p is 1, 2, 3, 4, or 5;
AA² is absent, or represents 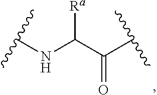
wherein R^{a} is selected from; H, optionally substituted C₁₋₆ alkyl, (C₃-C₁₀ carbocyclyl)C₁₋₆ alkyl, (C₆-C₁₀ aryl)C₁₋₆ alkyl, (C₆-C₁₀ aryl)C₁₋₄ alkyl, (C₁-C₁₀ heterocyclyl)C₁₋₆ alkyl, (C₂₋₆ alkynyl)C₁₋₄ alkyl, C₂₋₆ alkenyl, C₂₋₆ alkynyl, and C₆-C₁₀ aryl, in each of which optionally one or more of the carbon atoms can be replaced by heteroatoms; or wherein R^{a} is a side chain derived from a proteinogenic or non-proteinogenic amino acid;
Z is C(O), O, CH₂, NR′, C═O, C═S, C=NR′, HCR′ and R′CR′, with the proviso that two Os are not directly adjacent to each other;
Y is selected from: a single bond; a linking group; and, an optionally substituted C₁₋₁₆ aliphatic group, in which optionally one or more carbon atoms can be replaced by heteroatoms, and which can be saturated and optionally contain one or more double or triple bonds;
W is:
(i) a bicyclic optionally substituted 3- to 12 membered heterocyclyl group; or
(ii) a monocyclic optionally substituted 3- to 12 membered heterocyclyl group; or
(iii) a monocyclic or bicyclic optionally substituted 3- to 12 membered carbocyclyl group;
V is a single bond or a linking group which is a C₁₋₁₆ aliphatic group, in which optionally one or more carbon atoms can be replaced by heteroatoms, which can be saturated and optionally contain one or more double or triple bonds, and which is optionally substituted by one or more substituents selected from OH, SH, NH₂, halogen, cyano, oxo, alkyl, cycloalkyl, aryl and heteroaryl;
U is single bond or C(O), O, CH₂, NR′, C═O, C═S, C=NR′, HCR′ and R′CR′; and
each R′ is independently H or selected from: H, SH, NH₂, halogen, cyano, carboxy, C₁₋₆-alkyl, O(C₁₋₆ alkyl), S(C₁₋₆-alkyl), C₂₋₆ alkenyl, C₂₋₆ alkynyl, C₁₋₆ heteroalkenyl, C₁₋₆ heteroalkynyl, C₃₋₁₀ cycloalkenyl, C₁₋₁₀ cycloheteroalkenyl, C₆₋₁₀ aryl, and (C₆₋₁₀ aryl)C₁₋₆ alkyl, each of which being optionally substituted with from 1 to 3 substituents selected from —OH, oxo and halo, and optionally connected to an atom in Z, Y, W or V.

2. The compound of claim 1, wherein V is a is a single bond or is selected from: 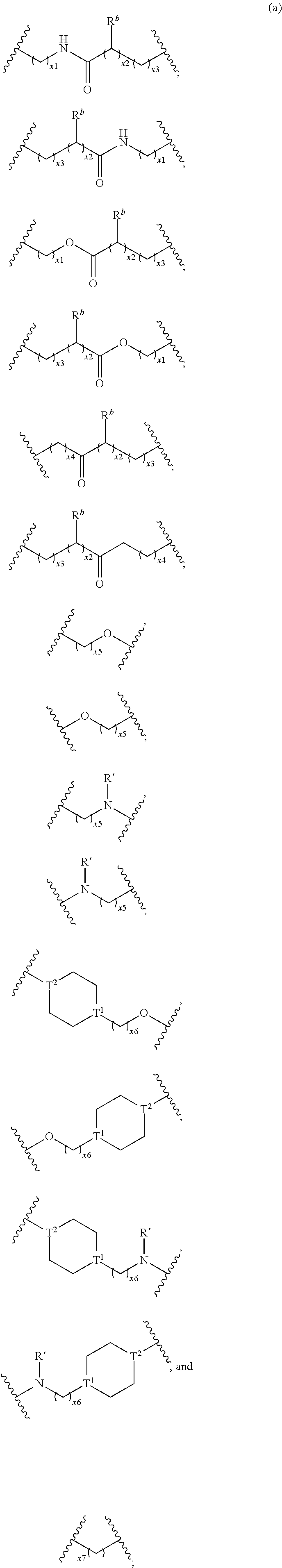
wherein R^{b} is H or a C₁₋₆ alkyl, C₂₋₆ alkenyl, C₂₋₆ alkynyl, —CH₃, —CH₂OH, —CH₂CH₂SCH₃, —C₂H₅, —CH₂CH₂CH₃, —CH₂CH₂CH₂CH₃, —CH (CH₃)₂, -Ph, or C₆-C₁₀ aryl group optionally substituted by one or more substituents selected from OH, SH, NH₂, halogen, cyano, alkyl, cycloalkyl, aryl and heteroaryl;
T¹ and T² are each independently selected from CR′ and N;
x1 is an integer of 0 to 14;
x2 is an integer of 0 to 6;
x3 is an integer of 0 to 14;
x4 is an integer of 0 to 14;
x5 is an integer of 0 to 15;
x6 is an integer of 0 to 11;
x7 is an integer of 1 to 16;
x1+x2+x3 is 14 or less;
x2+x3+x4 is 15 or less; or
b) single bond,

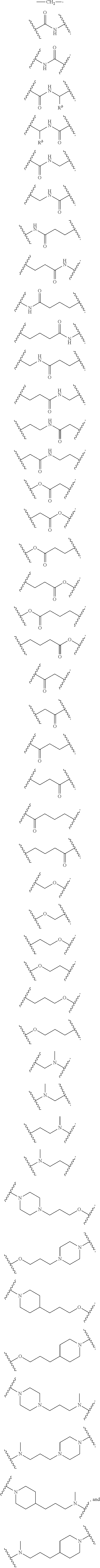

3. (canceled)

4. The compound of claim 1, wherein the group 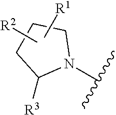
has the structure

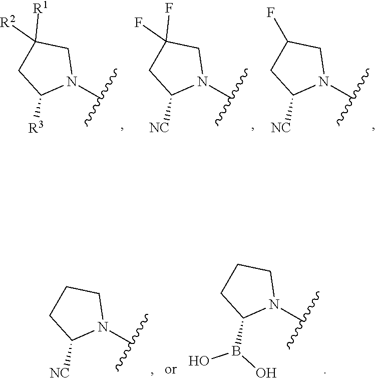

5. (canceled)

6. (canceled)

7. The compound of claim 1, wherein:
(a) AA¹ has the structure: 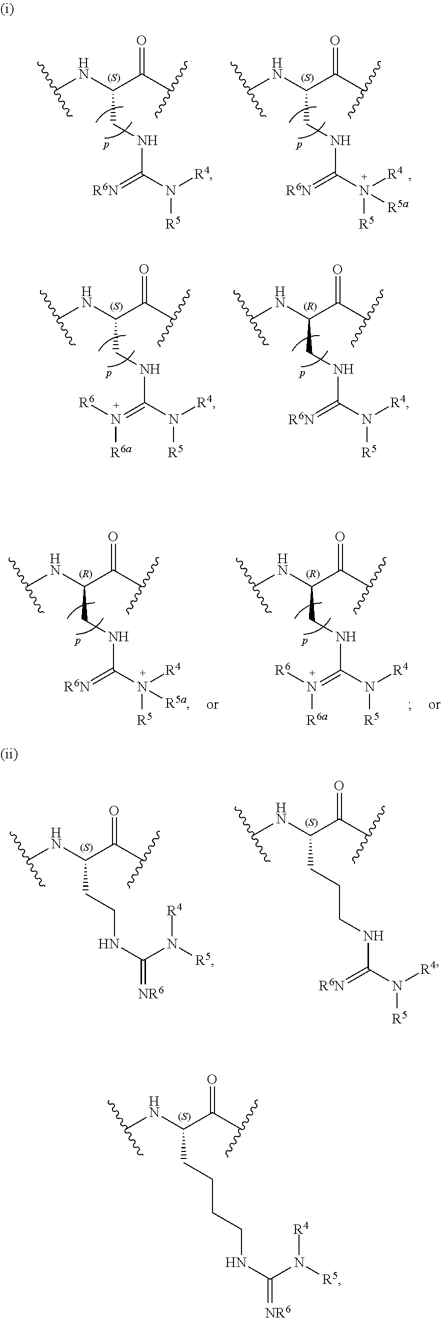 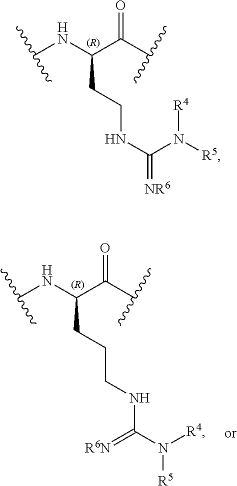 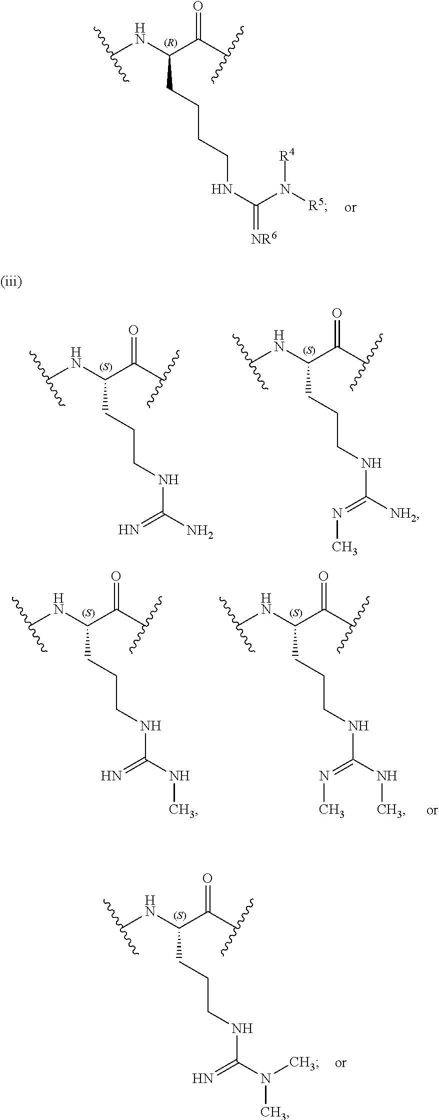 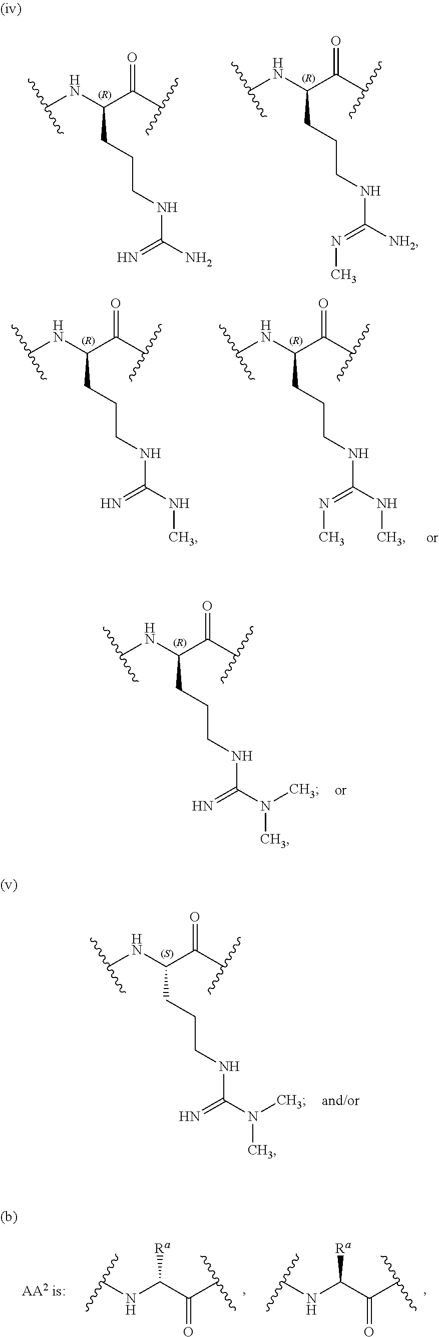
wherein R^{a} is —CH₃, —CH(CH₃)₂, —CH₂C≡CH, —((CH)₂)₄NH₂, or —CH₂-Ph; or

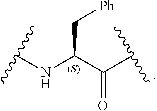

8-12. (canceled)

13. The compound of claim 1, wherein Y is independently selected from:
(a) single bond, 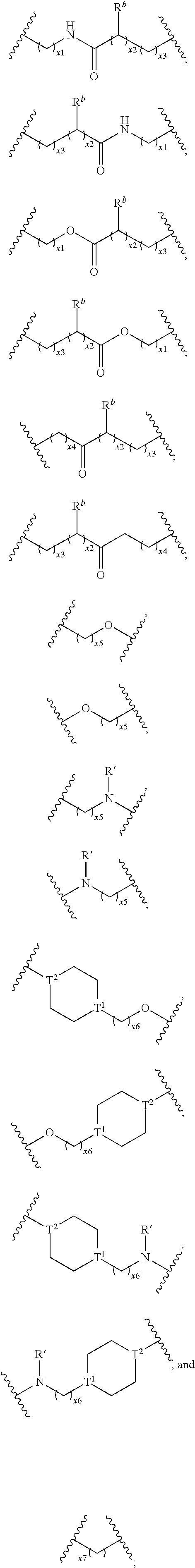
wherein R^{b} is selected from: H; an optionally substituted C₁₋₆ alkyl, C₂₋₆ alkenyl, C₂₋₆ alkynyl or C₆-C₁₀ aryl group, in which optionally one or more of the carbon atoms can be replaced by heteroatoms; a side chain derived from a proteinogenic or non-proteinogenic amino acid; and —CH₃, —CH₂OH, —CH₂CH₂SCH₃, —C₂H₅, —CH₂CH₂CH₃, —CH₂CH₂CH₂CH₃, —CH(CH₃)₂, and -Ph;
T¹ and T² are each independently selected from CR′ and N;
x1 is an integer of 0 to 14;
x2 is an integer of 0 to 6;
x3 is an integer of 0 to 14;
x4 is an integer of 0 to 14;
x5 is an integer of 0 to 15;
x6 is an integer of 0 to 11;
x7 is an integer of 1 to 16;
x1+x2+x3 is 14 or less;
x2+x3+x4 is 15 or less; or
(b) single bond, 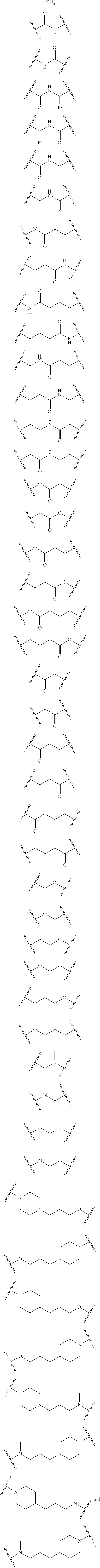
or
(c) single bond,

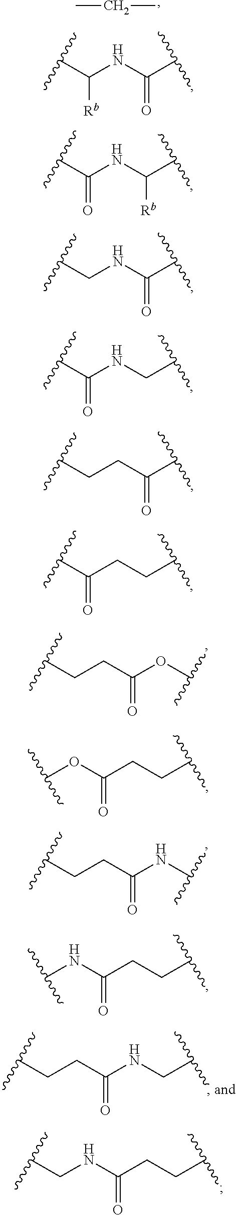

14. (canceled)

15. (canceled)

16. The compound of claim 1, wherein W is:
(a) monocyclic or bicyclic C₃₋₁₀ heteroaryl, or monocyclic or bicyclic optionally substituted C₆-C₁₀ aryl; or

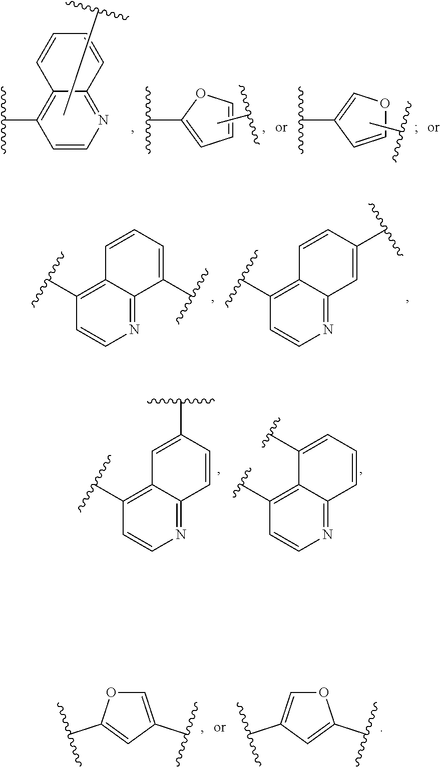

17-19. (canceled)

20. The compound of claim 1, wherein A or 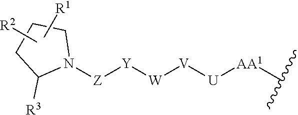
is represented by any one of the following structures:

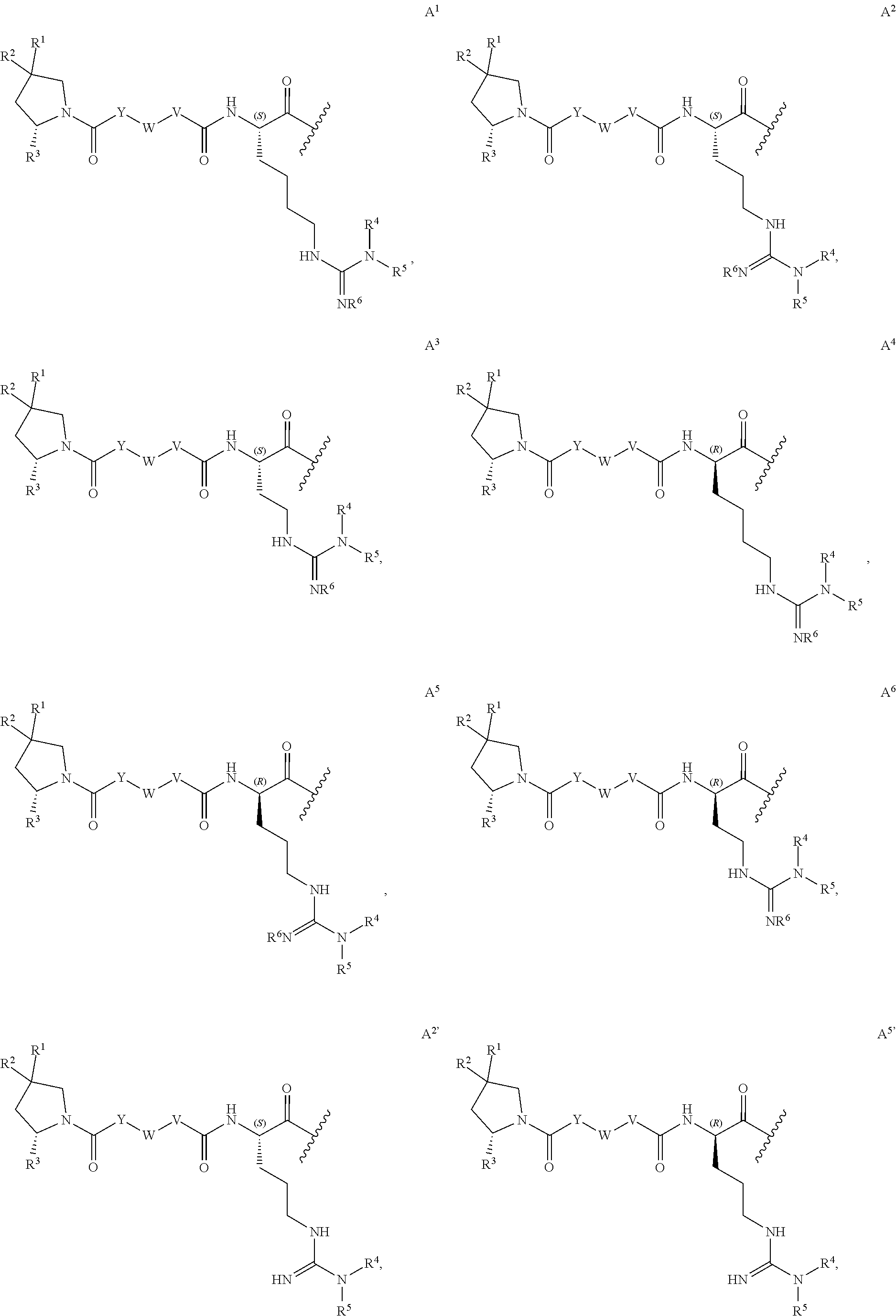

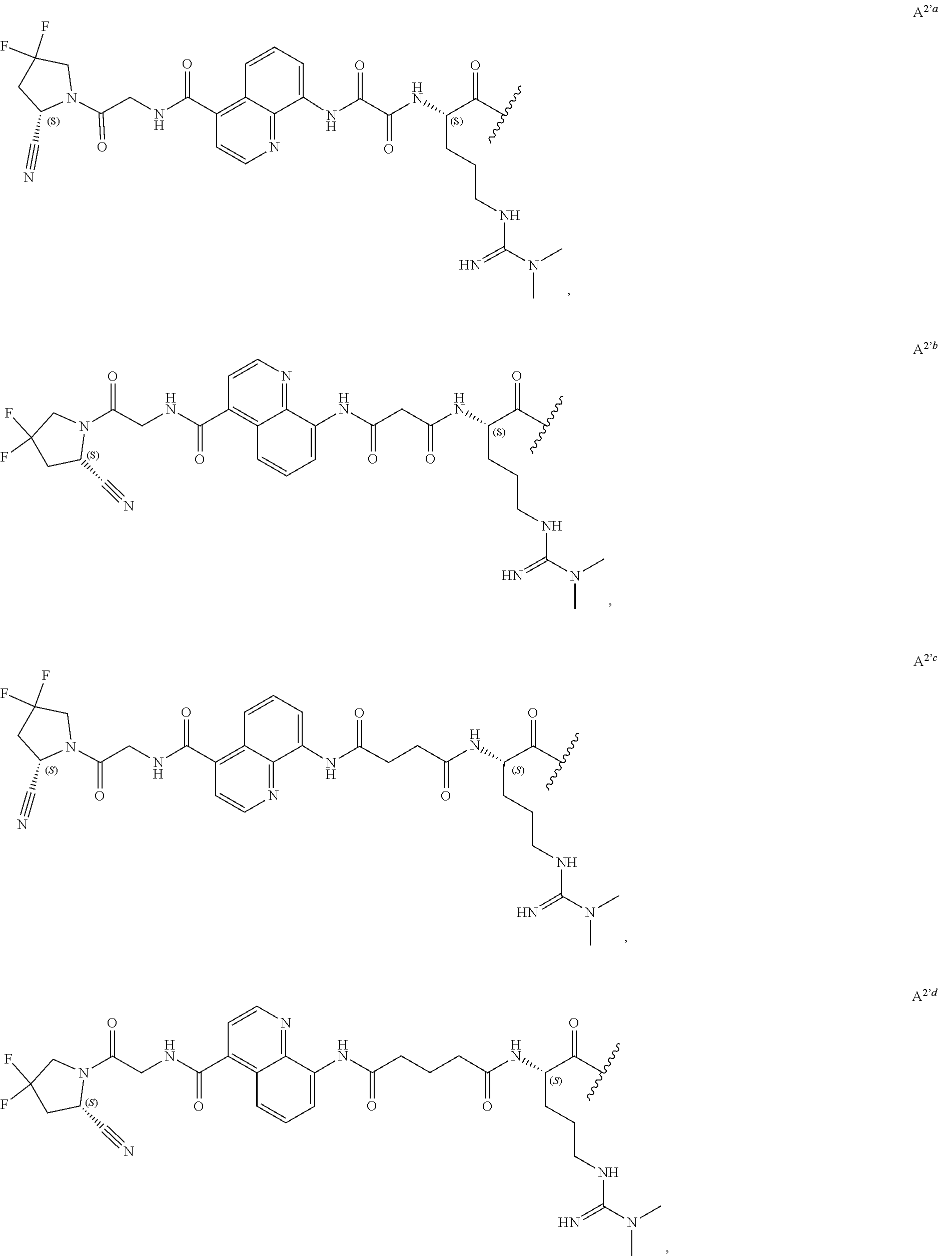

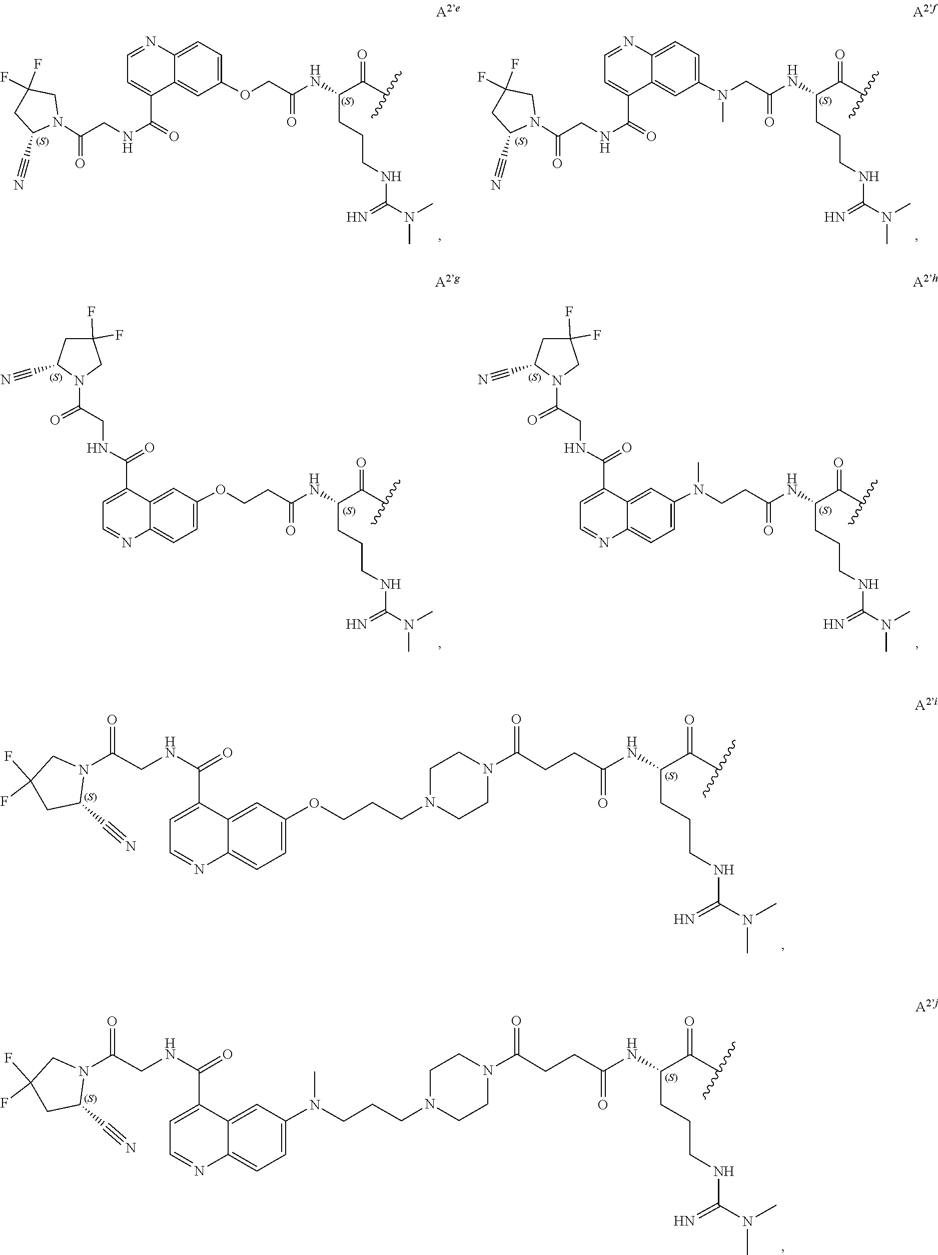

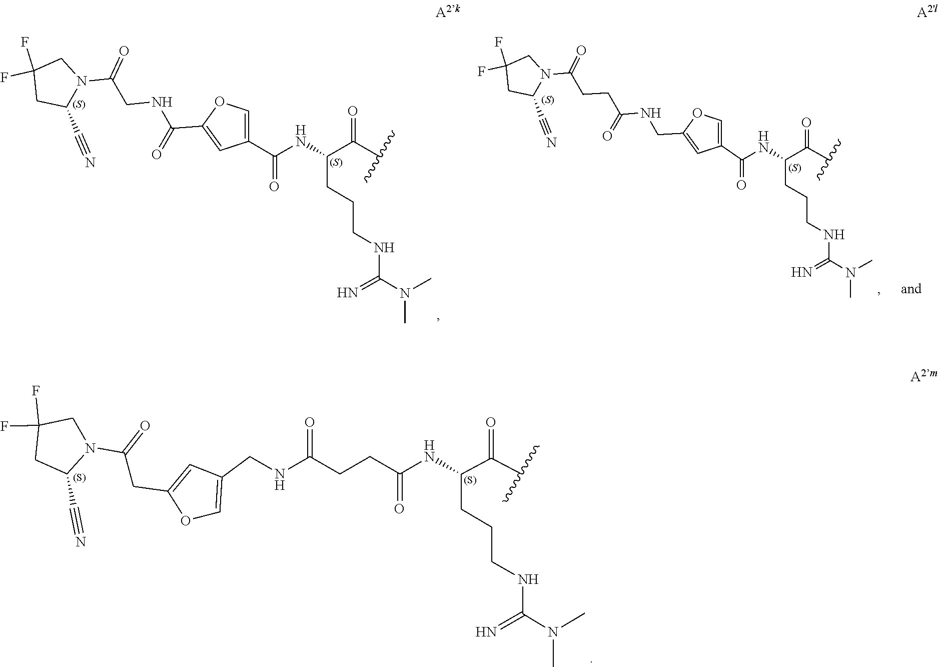

21-24. (canceled)

25. The compound of claim 1, wherein the compound structure further comprises at least one moiety C being an atom, a molecule or a particle, and/or is a therapeutic or diagnostic agent

26. The compound of claim 25, wherein the compound is represented by the following Formula I, Ia, or Ib, 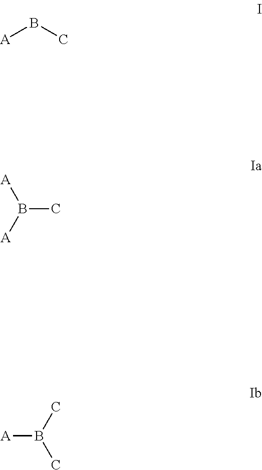
B is a single bond or an optionally substituted C₁₋₅₀ aliphatic group, in which optionally one or more carbon atoms can be replaced by a heteroatom, a C₃₋₁₂ carbocyclic or a C₁₋₁₂ heterocyclic group, and which can be saturated optionally contain one or more double or triple bonds.

27. The compound of claim 26, wherein B has a structure selected from:
(a) the following general Formulae II-V, Ha-Va, or IIb-Vb: 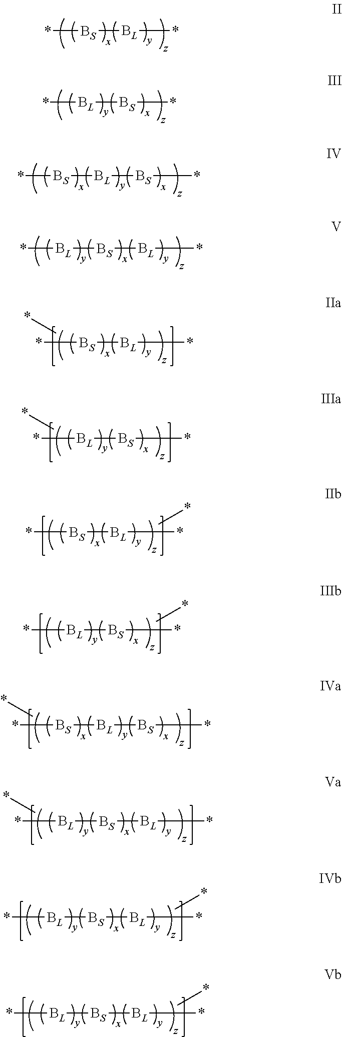
wherein
each x is 0, 1, 2, 3, 4, 5, 6, 7, 8, 9, or 10;
each y is 0, 1, 2, 3, 4, 5, 6, 7, 8, 9, or 10;
each z is 0, 1, 2, 3 or 4;
with the proviso that in Formulae Ha-Va and IIb-Vb, z and at least one of x and y is not 0;
* represents a point of attachment to a moiety A;
* represents a point of attachment to a moiety C; and
each of B_{S} and B_{L} is independently selected from alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, aminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide, tetrapeptide, each of which is optionally substituted, 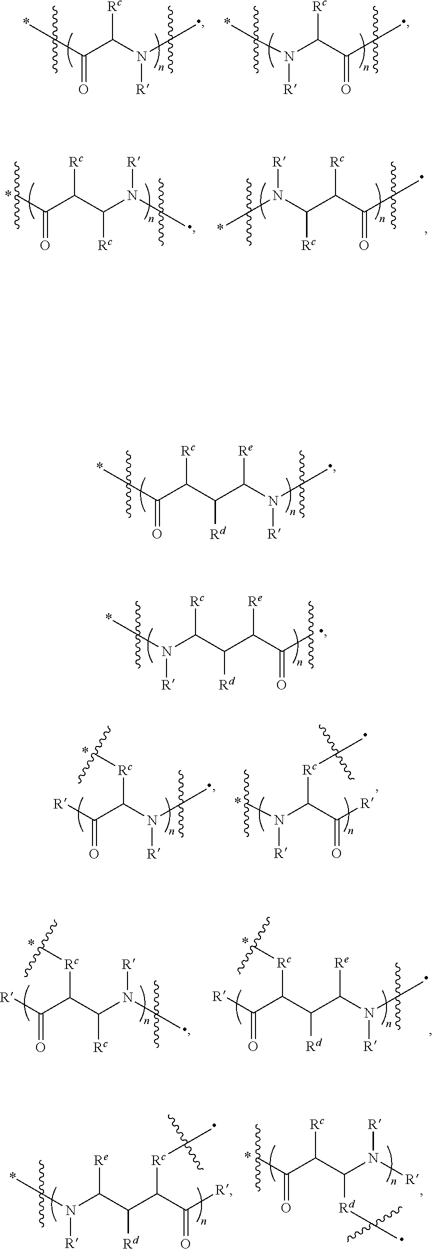 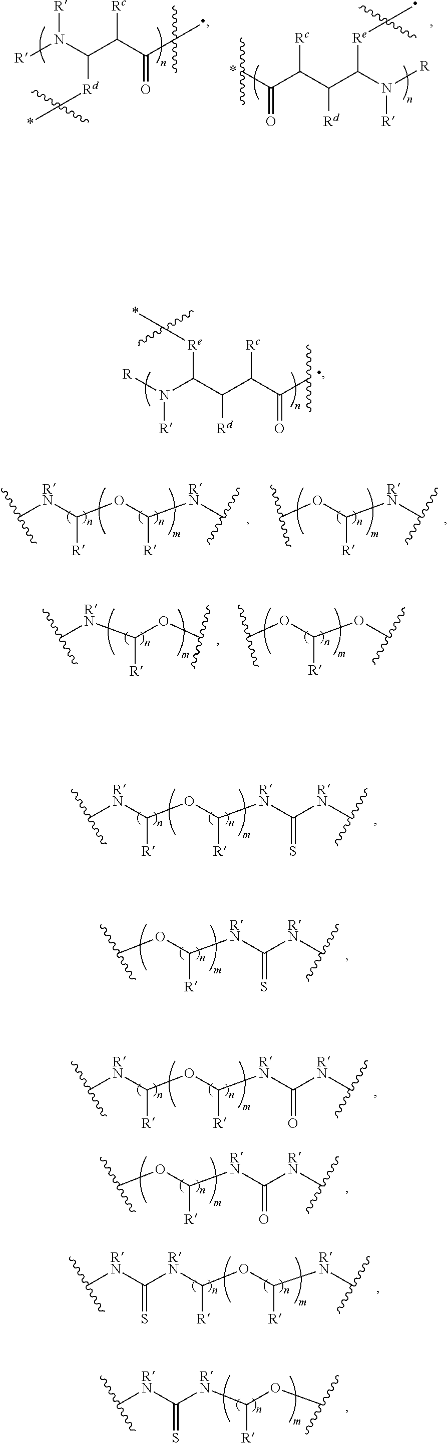 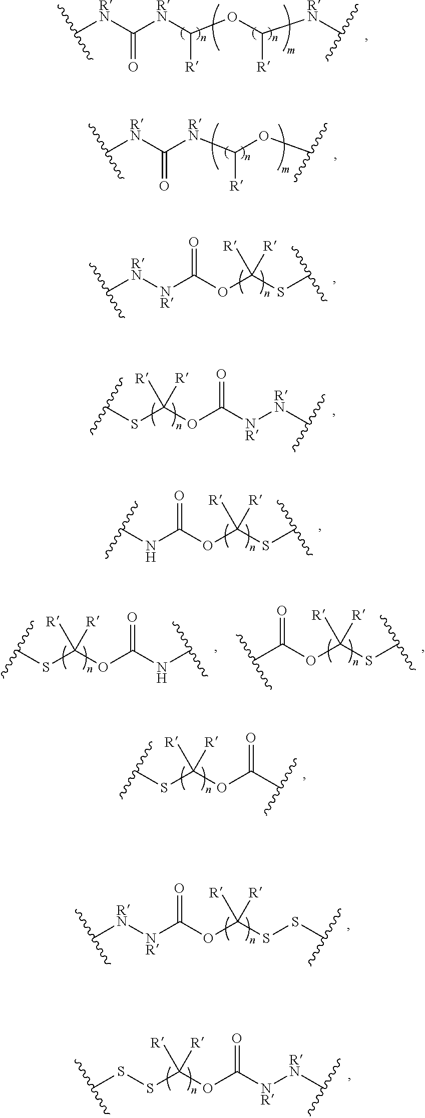 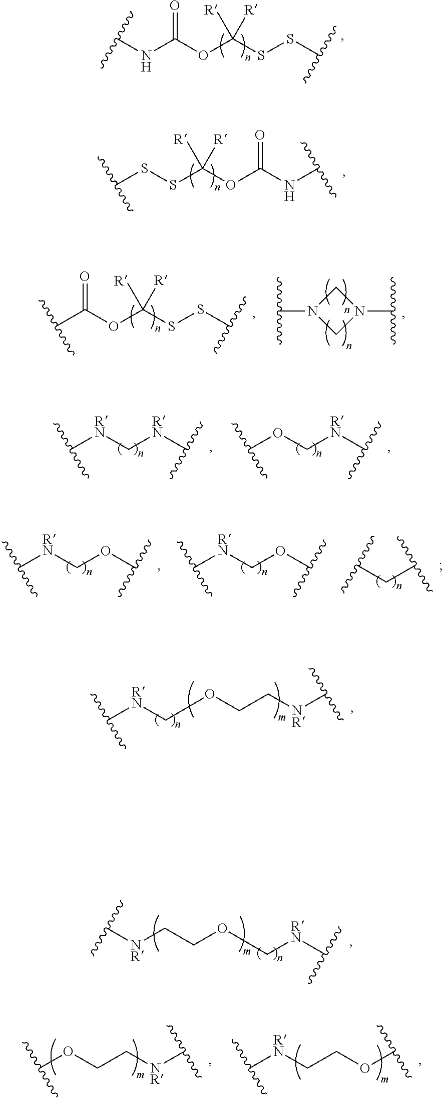 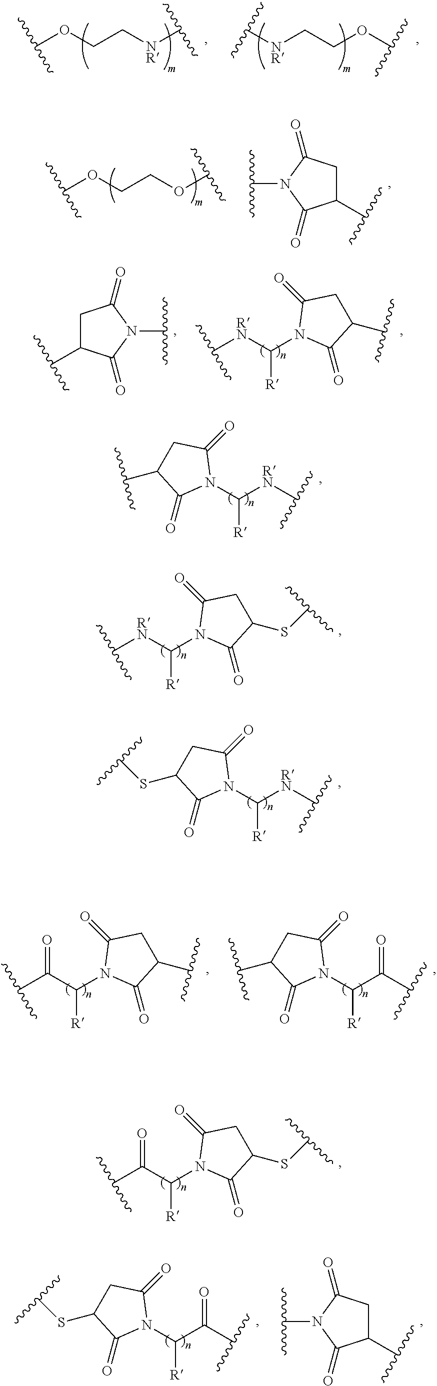 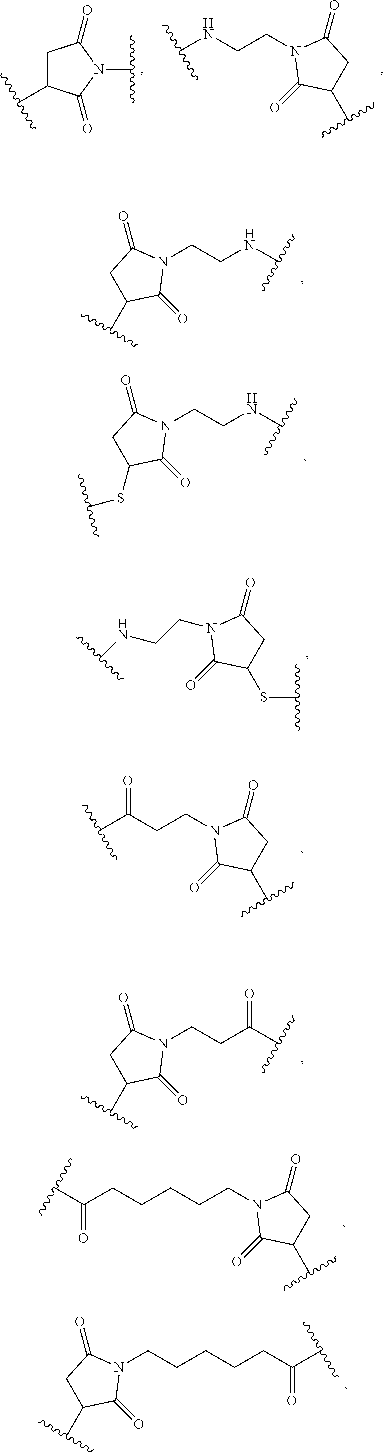 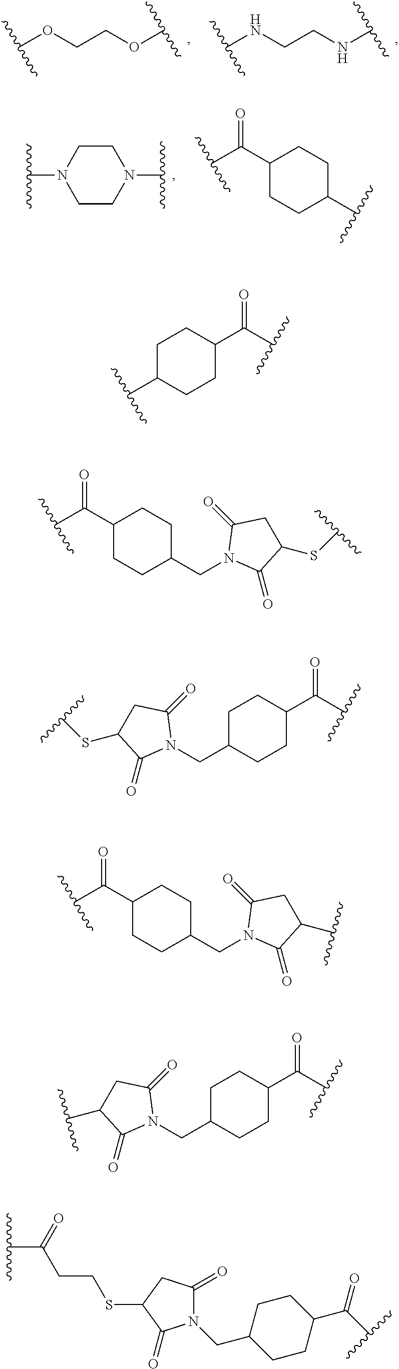 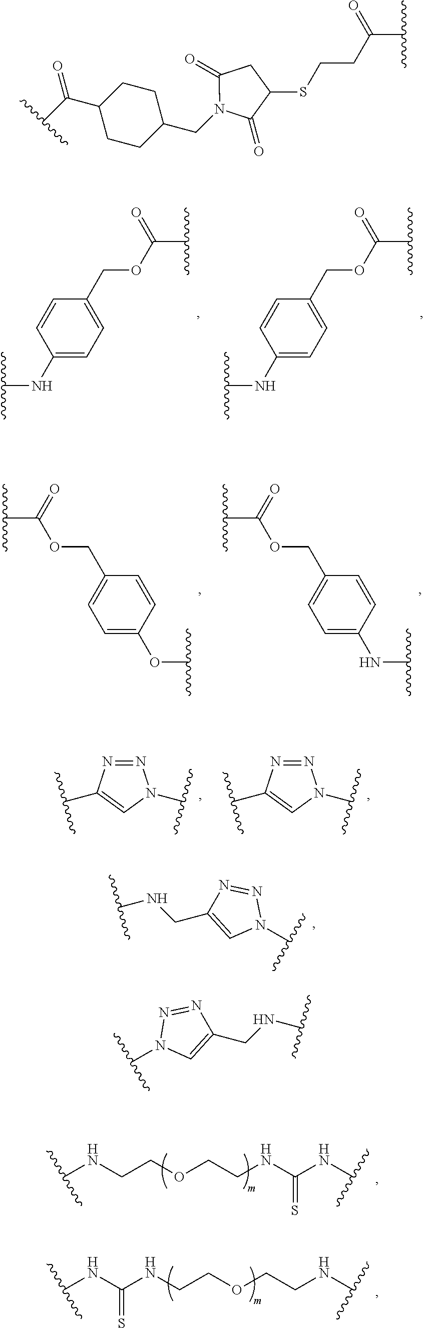 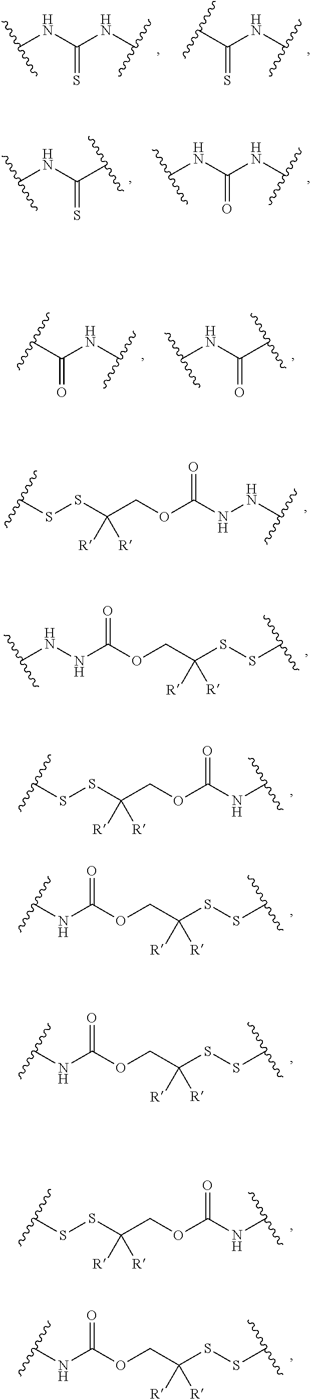 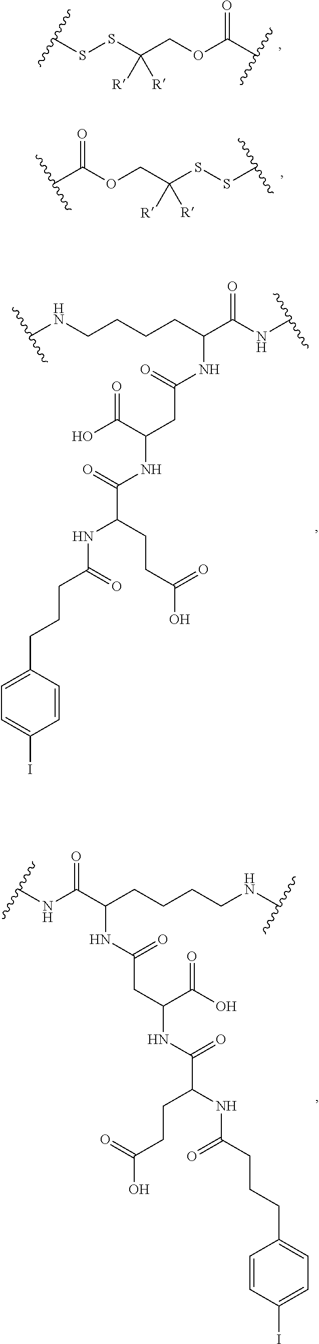 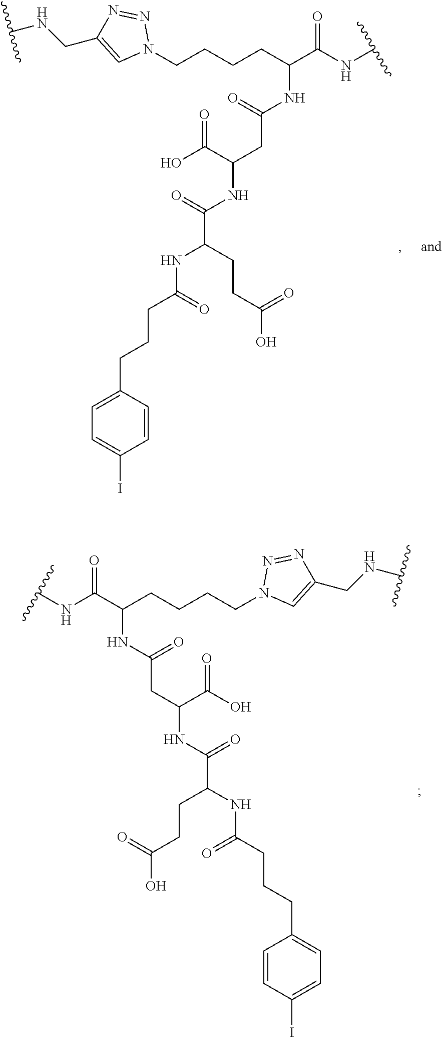 [SMILES: *NCc1cn(CCCCC(NC(=O)CC(NC(=O)C(CCC(=O)O)NC(=O)CCCc2ccc(I)cc2)C(=O)O)C(=O)N*)nn1 ; *NCc1cn(CCCCC(NC(=O)CC(NC(=O)C(CCC(=O)O)NC(=O)CCCc2ccc(I)cc2)C(=O)O)C(=O)N*)nn1]
wherein in each of the above structures:
each n is independently 0, 1, 2, 3, 4, 5, 6, 7, or 8;
each m is independently 0, 1, 2, 3, or 4:
each R^{c}, R^{d}, and R^{e} is independently selected from: H, optionally substituted C₁₋₆ alkyl, (C₃-C₁₀ carbocyclyl)C₁₋₆ alkyl, (C₆-C₁₀ aryl)C₁₋₆ alkyl, (C₁-C₁₀ heterocyclyl)C₁₋₆ alkyl, C₂₋₆ alkenyl, C₂₋₆ alkynyl, and C₆-C₁₀ aryl, in each of which optionally one or more of the carbon atoms can be replaced by heteroatoms; or side-chain residues of proteinogenic or a non-proteinogenic amino acids;
each * represents a point of attachment for which the shortest path to a moiety A comprises less atoms than that for •; and each • represents a point of attachment for which the shortest path to a moiety C comprises less atoms than that for *, with the proviso that when n is >1 and a respective point of attachment is indicated on any one of R^{c}, R^{d} and R^{e}, then it can be independently present in one or more of the peptide monomeric units, or in one peptide monomeric unit most distant from the other point of attachment indicated in the respective structure,
wherein each of the above structures optionally comprises a further attachment point to a moiety A or C; or
(b) single bond, (B_{S})_{X}, 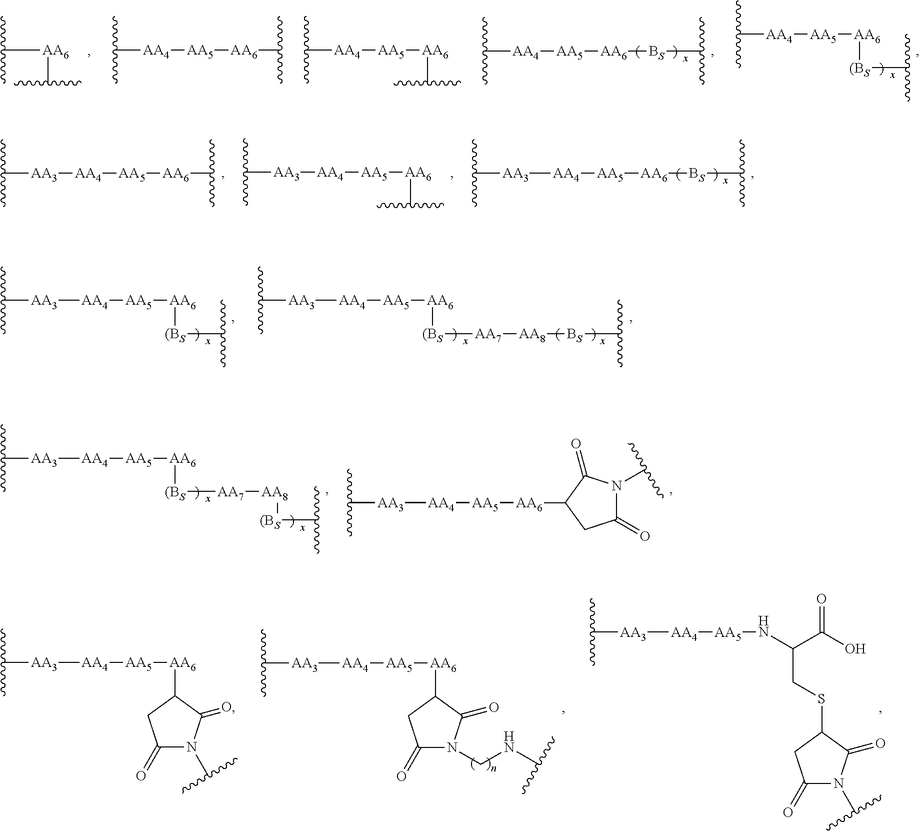 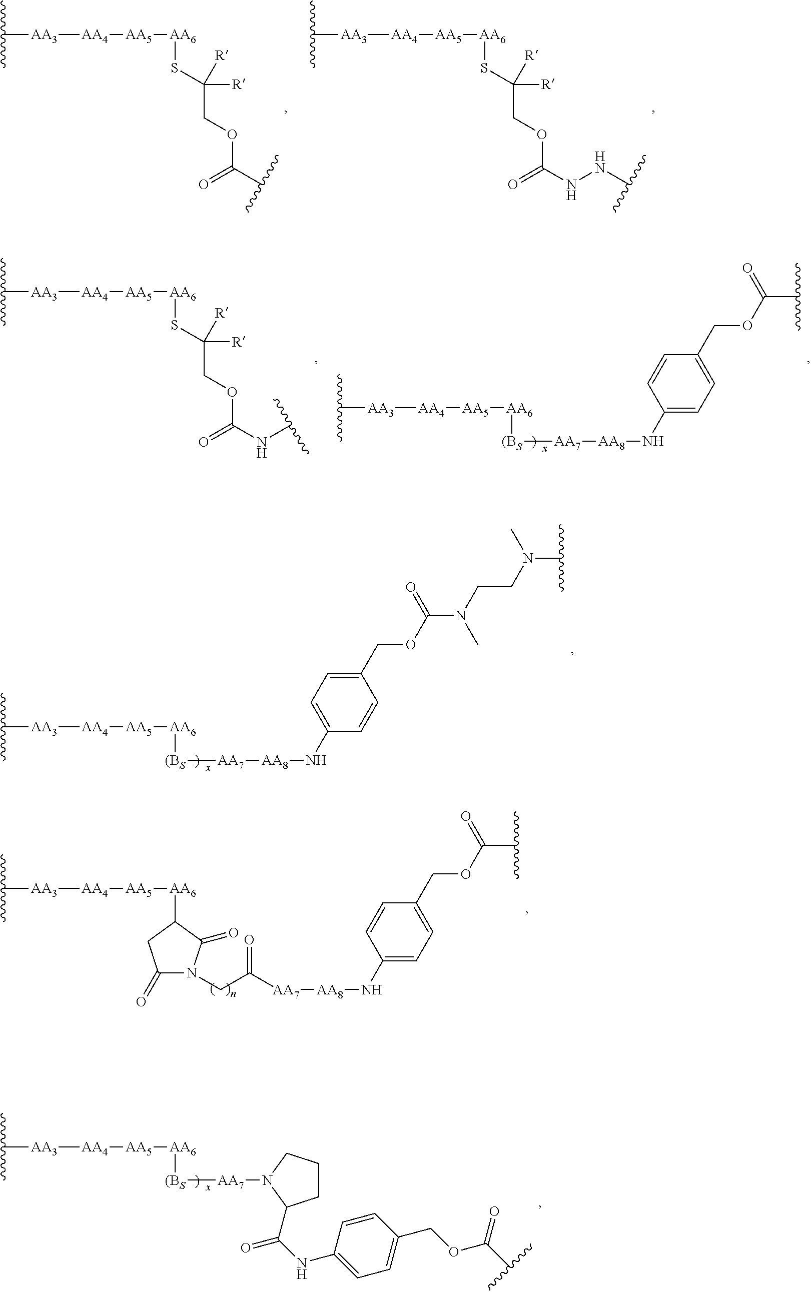 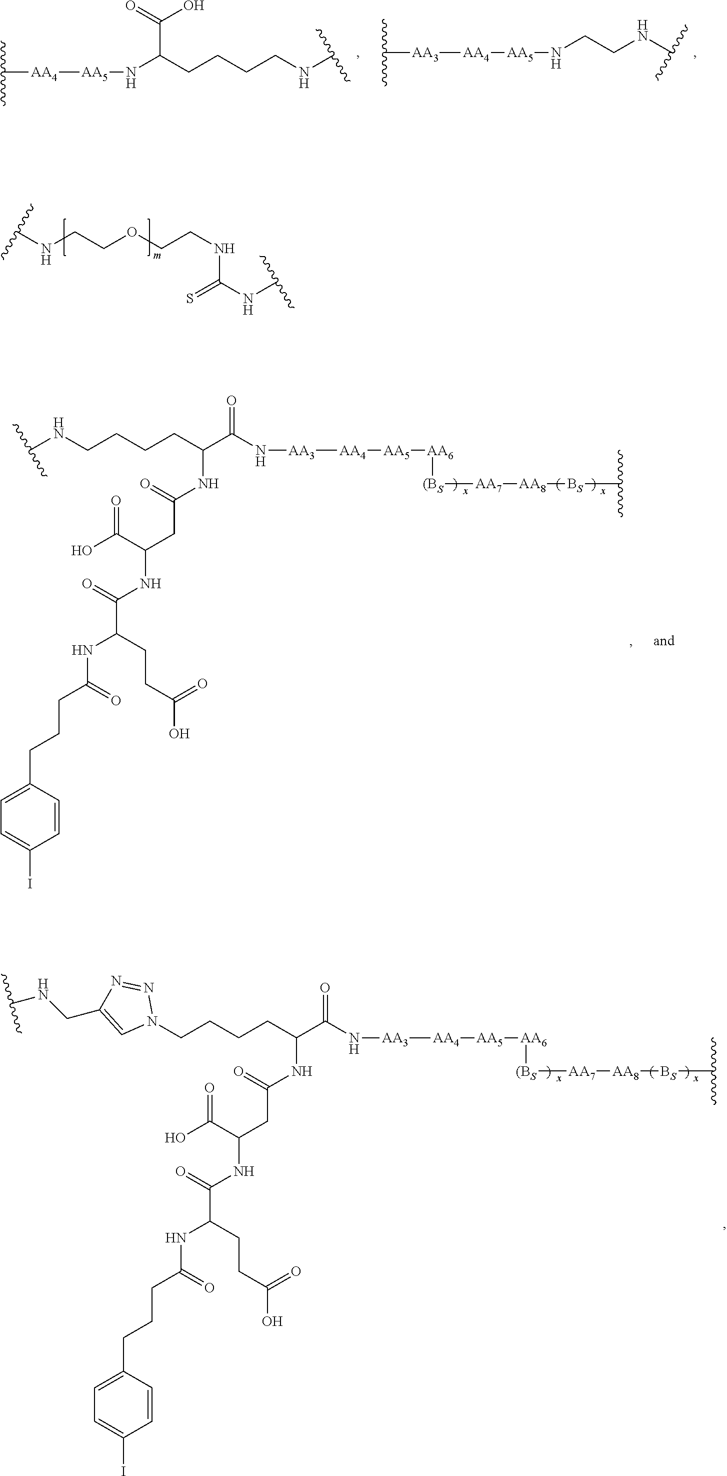
wherein:
(i) each of AA₃, AA₄, AA₅, AA₆, AA₇, and AA₈ represents a proteinogenic or non-proteinogenic amino acid, or is absent; or
(ii) each of AA₃, AA₄, AA₅, AA₆, AA₇, and AA₈ represents a proteinogenic or non-proteinogenic amino acid, or is absent, wherein each proteinogenic or non-proteinogenic amino acid independently represented by one of the following structures: 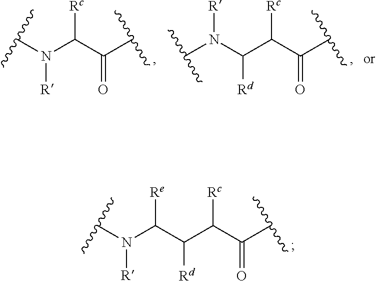
and/or AA₄ is an amino acid with a charged sidechain, and AA₇ is an amino acid with an aliphatic sidechain; or
(iii) AA₃ is selected from Asp, Glu, and Lys, or is absent:
AA₄ is selected from Arg, HomoArg, Lys, Asp, and Glu, or is absent:
AA₅ is selected from Asp, Glu, and Lys:
AA₆ is selected from Cys, Lys, Gly and Val:
AA₇ is selected from Gly, Ala, Val, Arg, Ile, Pro; and
AA₈ is selected from Pro and citrulline (Cit); or
iv) AA₃, AA₄, AA₅, AA₆, AA₇, and AA₈ are according to one of the sequences shown in the below table:

| AA₃ | AA₄ | AA₅ | AA₆ | AA₇ | AA₈ | AA₃ | AA₄ | AA₅ | AA₆ | AA₇ | AA₈ |
|---|---|---|---|---|---|---|---|---|---|---|---|
| — | — | — | Lys | — | — | | | | | | |
| Asp | Arg | Asp | Cys | — | — | Asp | Lys | Asp | Cys | Ala | Pro |
| Asp | Arg | Asp | Gly | — | — | Asp | Lys | Asp | Cys | Ala | Val |
| Asp | Lys | Asp | Lys | — | — | Asp | Lys | Asp | Cys | Arg | Pro |
| Asp | Lys | Asp | Val | — | — | Asp | Lys | Asp | Cys | Gly | Pro |
| Asp | Arg | Asp | Cys | Val | Ala | Asp | Lys | Asp | Cys | Ile | Pro |
| Asp | Arg | Asp | Cys | Val | Cit | Asp | Lys | Asp | Cys | Pro | Pro |
| Asp | HomoArg | Asp | Cys | Val | Cit | Asp | Lys | Asp | Cys | Val | Cit |
| Asp | Lys | Asp | Cys | any AA | Pro | Asp | Lys | Asp | Cys | Val | Pro |

(c) single bond,

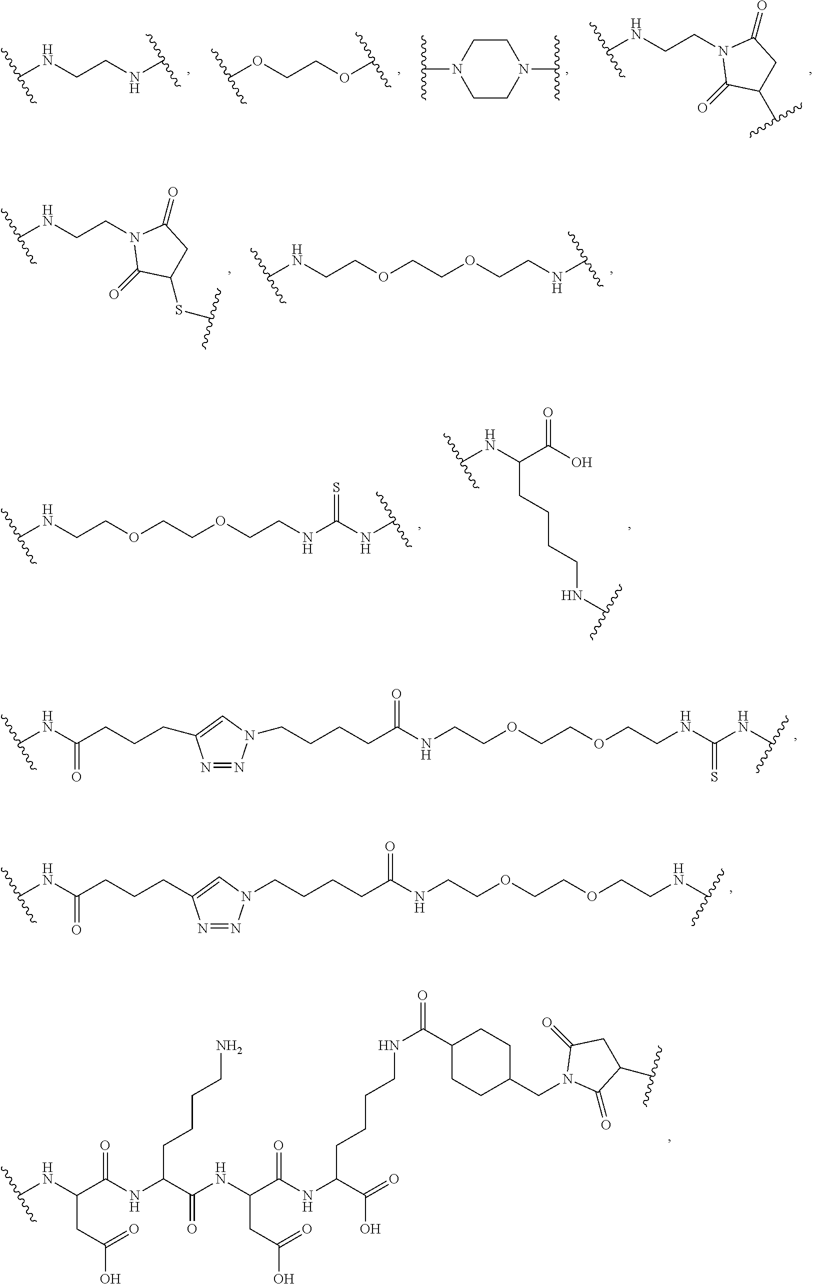

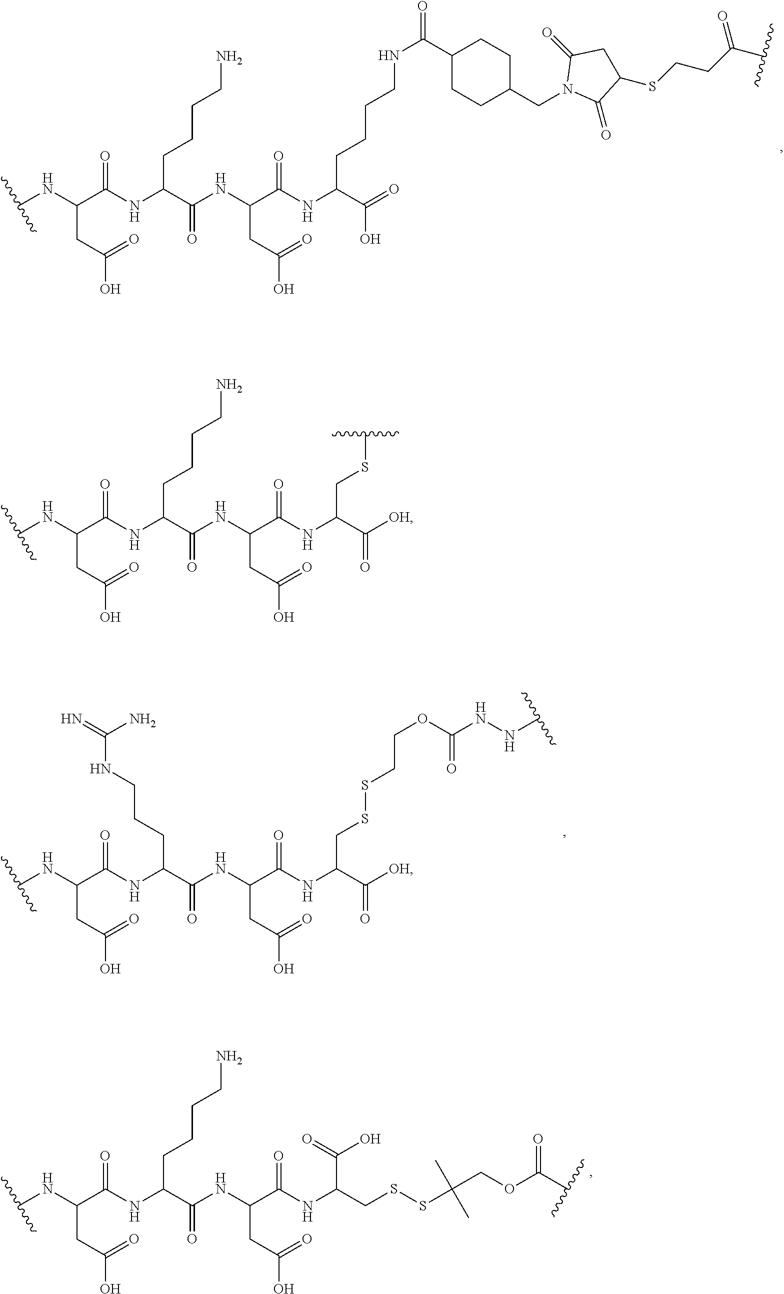 [SMILES: *NC(CC(=O)O)C(=O)NC(CCCCN)C(=O)NC(CC(=O)O)C(=O)NC(CCCCNC(=O)C1CCC(CN2C(=O)CC(SCCC(*)=O)C2=O)CC1)C(=O)O ; *NC(CC(=O)O)C(=O)NC(CCCCN)C(=O)NC(CC(=O)O)C(=O)NC(CS*)C(=O)O ; *NNC(=O)OCCSSCC(NC(=O)C(CC(=O)O)NC(=O)C(CCCNC(=N)N)NC(=O)C(CC(=O)O)N*)C(=O)O ; *NC(CC(=O)O)C(=O)NC(CCCCN)C(=O)NC(CC(=O)O)C(=O)NC(CSSC(C)(C)COC(*)=O)C(=O)O]

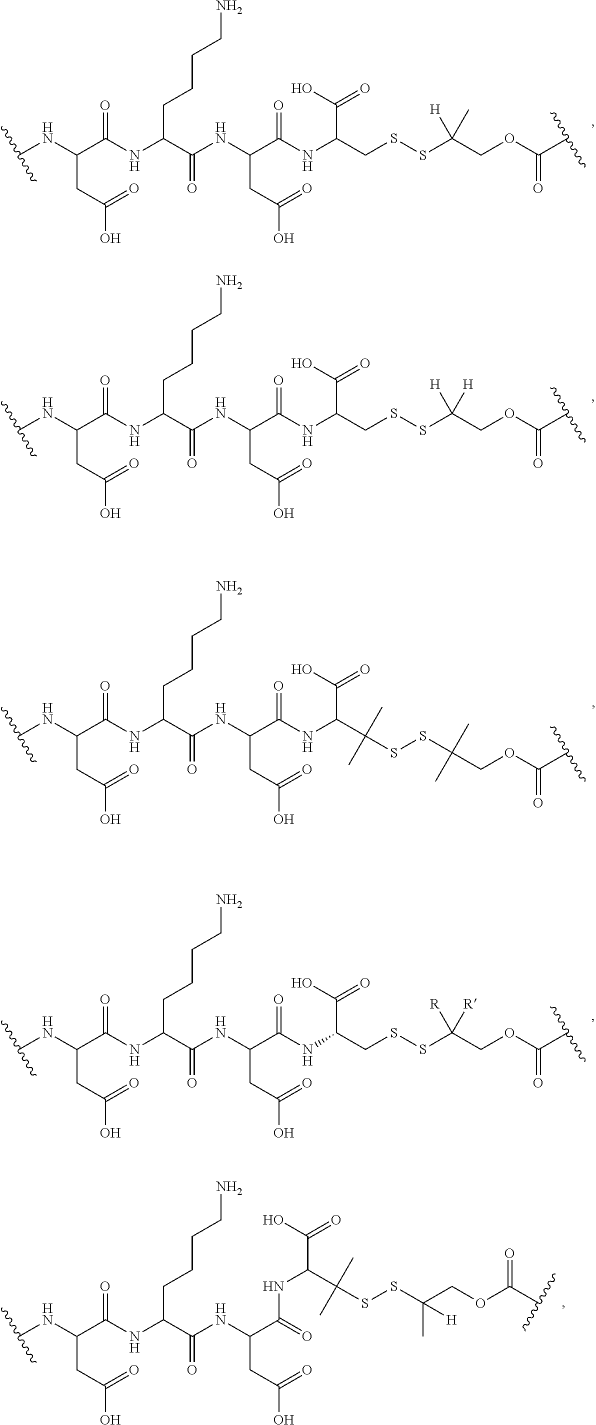

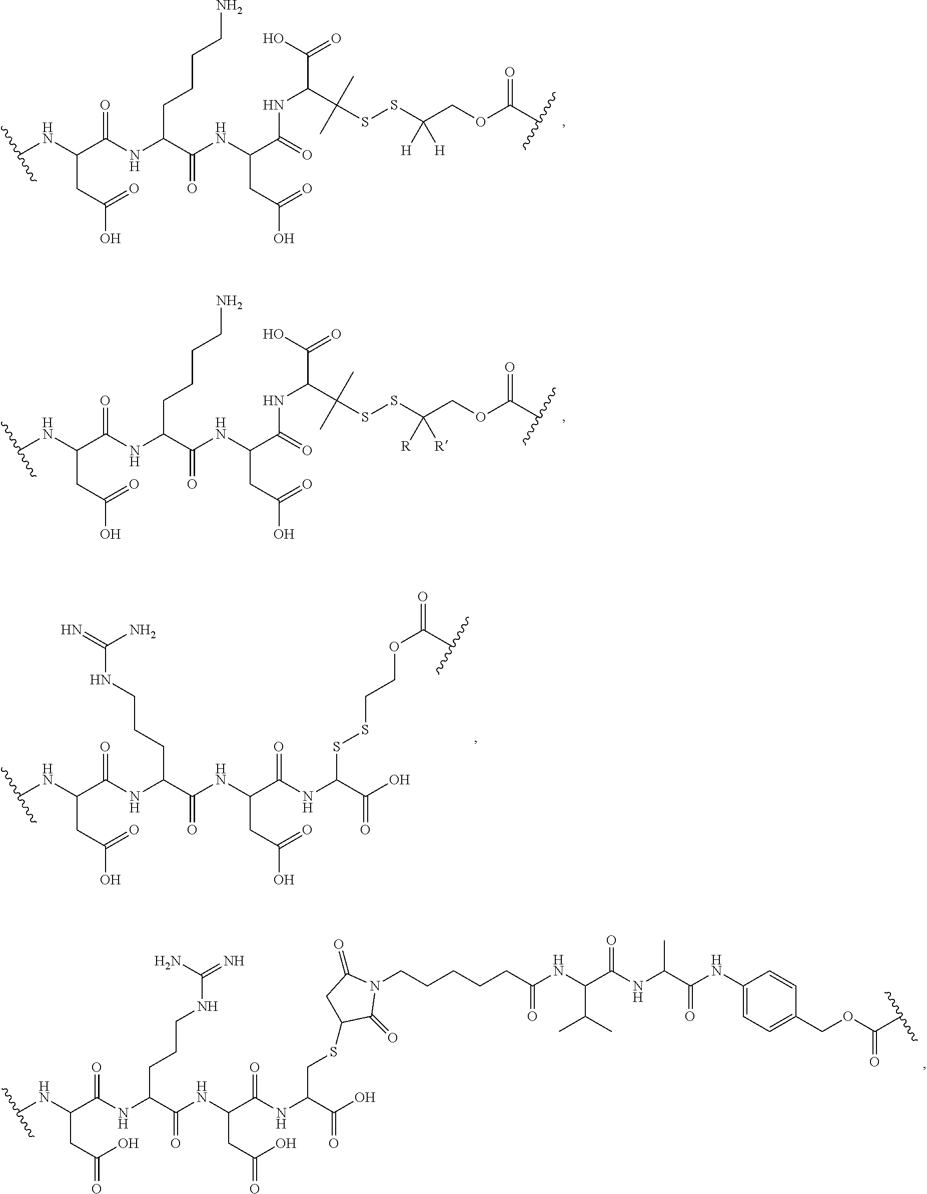

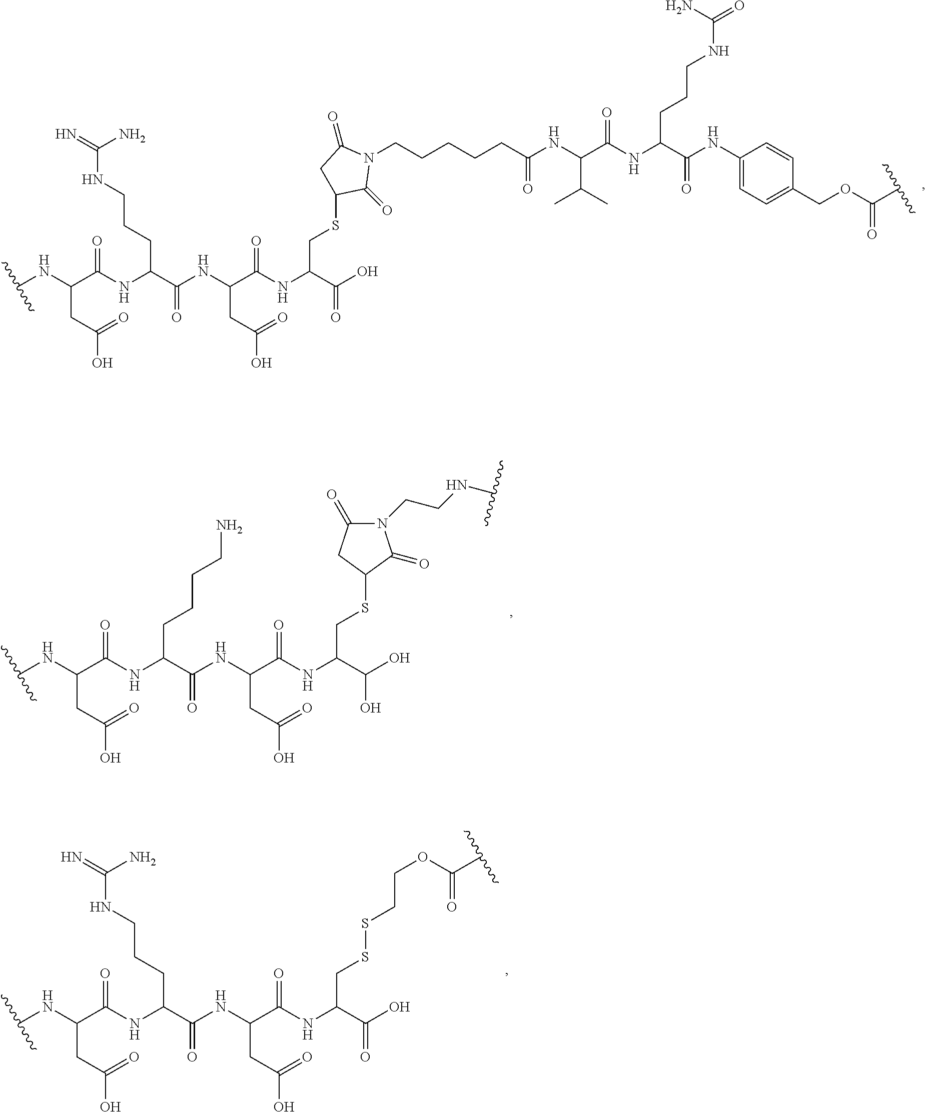 [SMILES: *NC(CC(=O)O)C(=O)NC(CCCNC(=N)N)C(=O)NC(CC(=O)O)C(=O)NC(CSC1CC(=O)N(CCCCCC(=O)NC(C(=O)NC(CCCNC(N)=O)C(=O)Nc2ccc(COC(*)=O)cc2)C(C)C)C1=O)C(=O)O ; *NCCN1C(=O)CC(SCC(NC(=O)C(CC(=O)O)NC(=O)C(CCCCN)NC(=O)C(CC(=O)O)N*)C(O)O)C1=O ; *NC(CC(=O)O)C(=O)NC(CCCNC(=N)N)C(=O)NC(CC(=O)O)C(=O)NC(CSSCCOC(*)=O)C(=O)O]

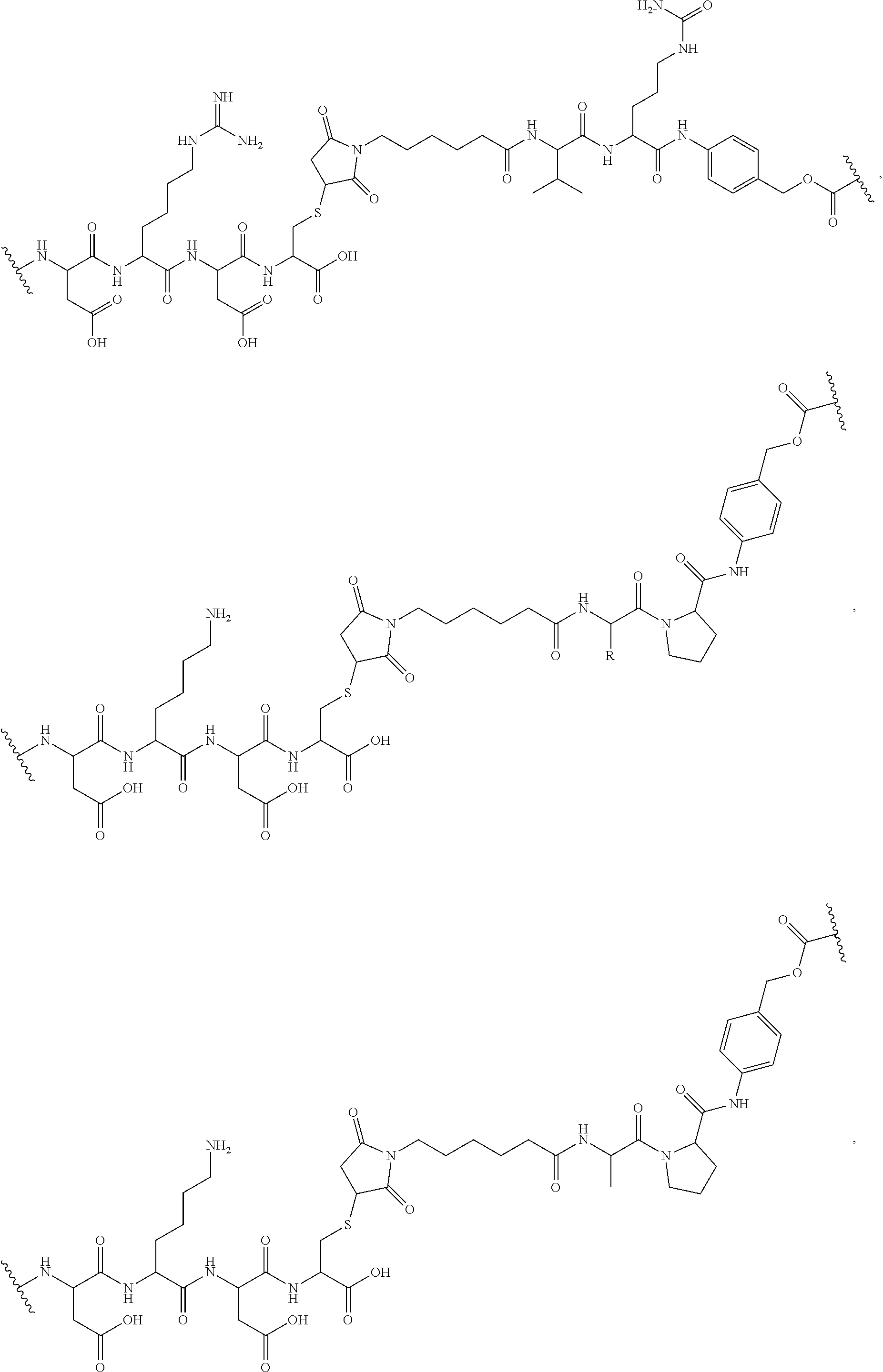

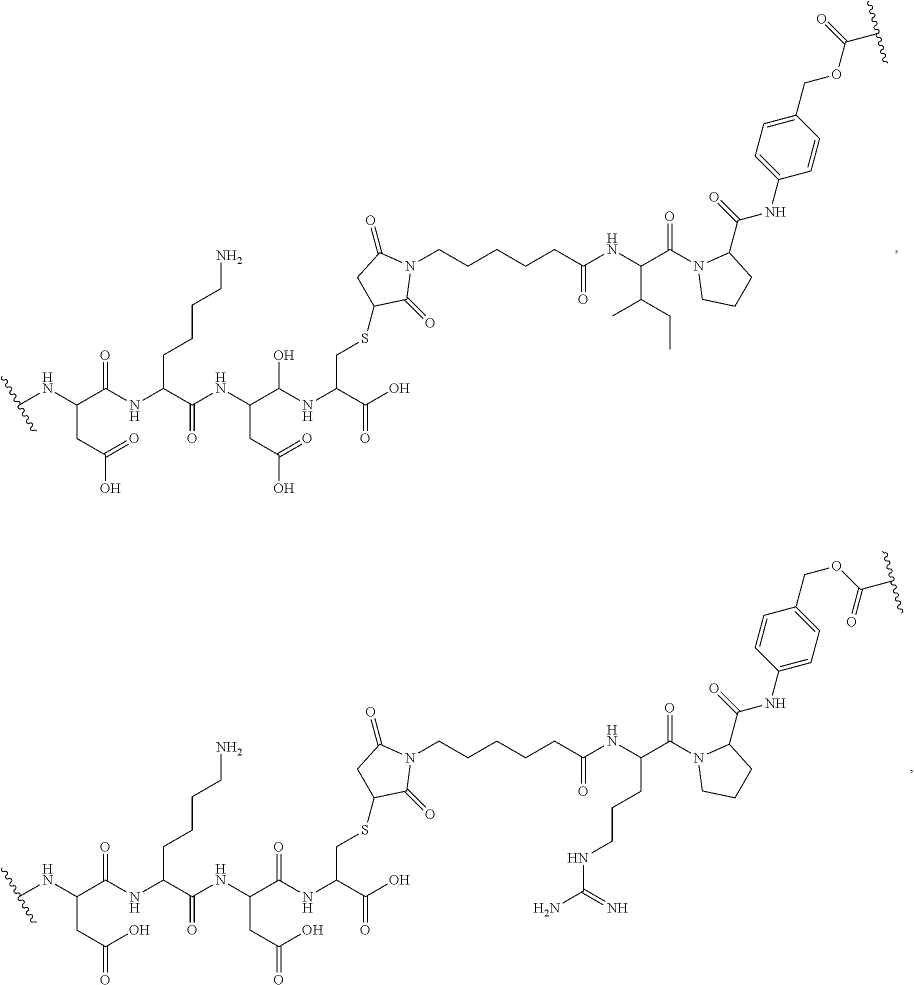 [SMILES: *NC(CC(=O)O)C(=O)NC(CCCCN)C(=O)NC(CC(=O)O)C(O)NC(CSC1CC(=O)N(CCCCCC(=O)NC(C(=O)N2CCCC2C(=O)Nc2ccc(COC(*)=O)cc2)C(C)CC)C1=O)C(=O)O ; *NC(CC(=O)O)C(=O)NC(CCCCN)C(=O)NC(CC(=O)O)C(=O)NC(CSC1CC(=O)N(CCCCCC(=O)NC(CCCNC(=N)N)C(=O)N2CCCC2C(=O)Nc2ccc(COC(*)=O)cc2)C1=O)C(=O)O]

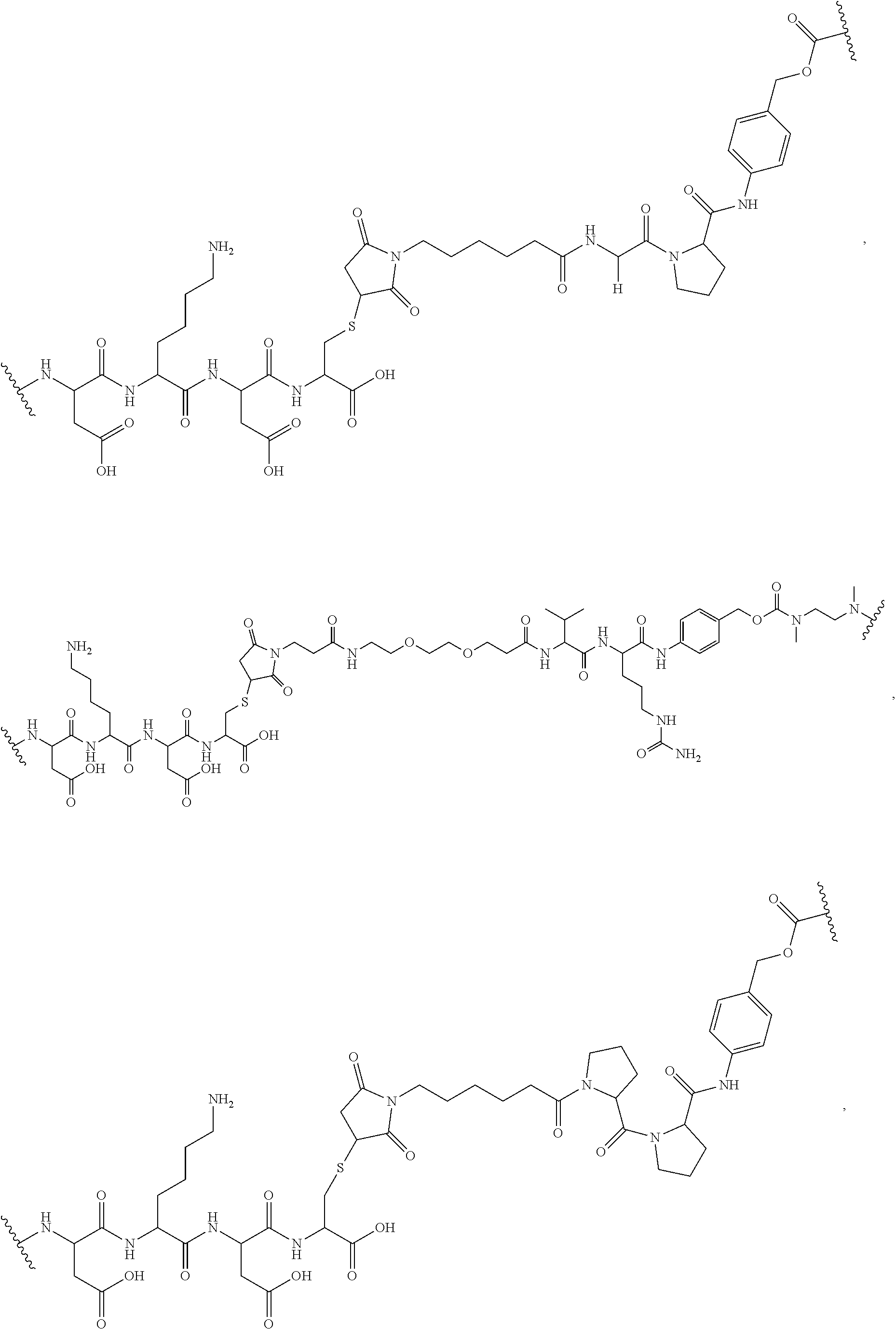 [SMILES: *NC(CC(=O)O)C(=O)NC(CCCCN)C(=O)NC(CC(=O)O)C(=O)NC(CSC1CC(=O)N(CCCCCC(=O)NCC(=O)N2CCCC2C(=O)Nc2ccc(COC(*)=O)cc2)C1=O)C(=O)O ; *NC(CC(=O)O)C(=O)NC(CCCCN)C(=O)NC(CC(=O)O)C(=O)NC(CSC1CC(=O)N(CCC(=O)NCCOCCOCCC(=O)NC(C(=O)NC(CCCNC(N)=O)C(=O)Nc2ccc(COC(=O)N(C)CCN(*)C)cc2)C(C)C)C1=O)C(=O)O ; *NC(CC(=O)O)C(=O)NC(CCCCN)C(=O)NC(CC(=O)O)C(=O)NC(CSC1CC(=O)N(CCCCCC(=O)N2CCCC2C(=O)N2CCCC2C(=O)Nc2ccc(COC(*)=O)cc2)C1=O)C(=O)O]

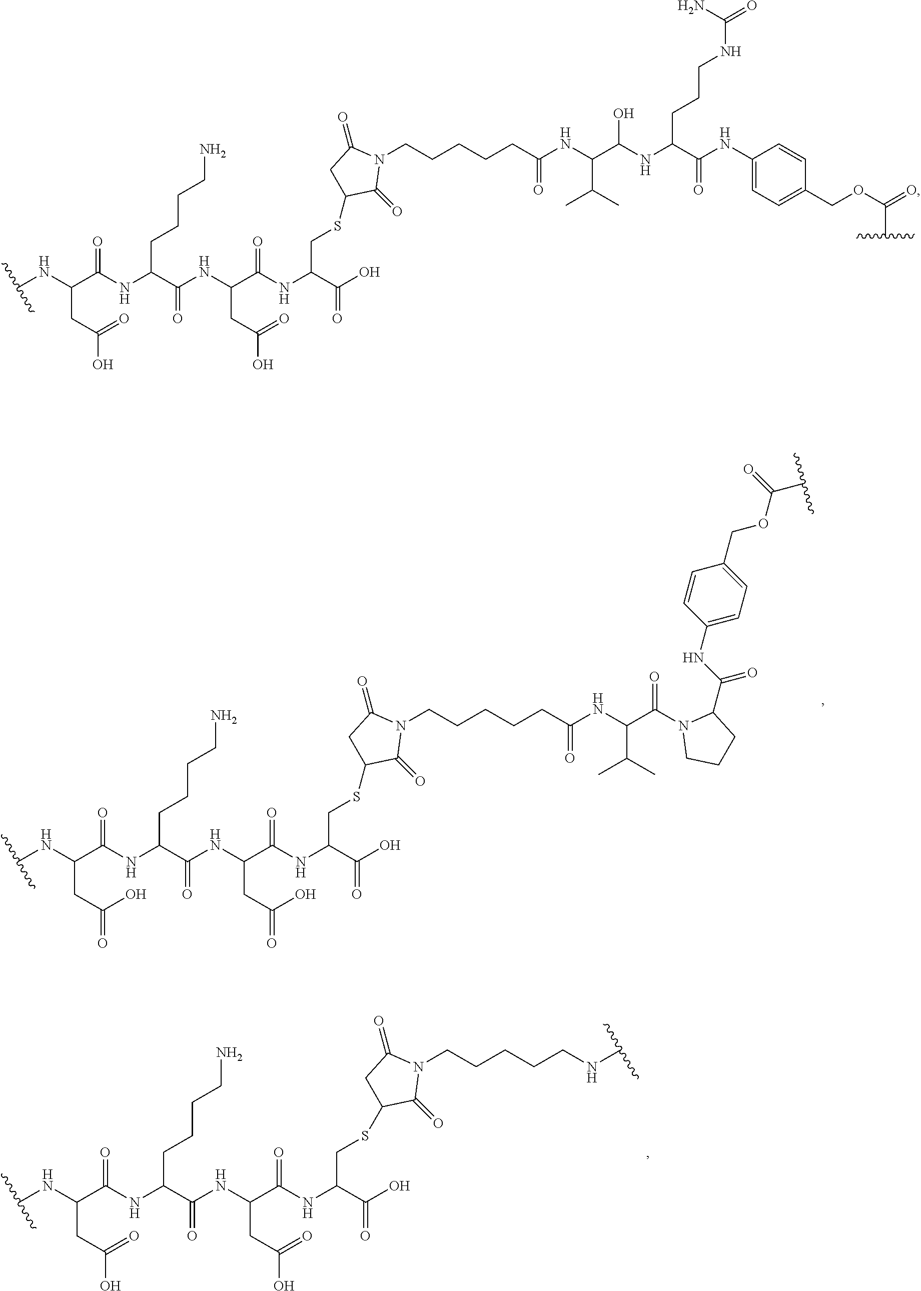 [SMILES: *NC(CC(=O)O)C(=O)NC(CCCCN)C(=O)NC(CC(=O)O)C(=O)NC(CSC1CC(=O)N(CCCCCC(=O)NC(C(C)C)C(O)NC(CCCNC(N)=O)C(=O)Nc2ccc(COC(*)=O)cc2)C1=O)C(=O)O ; *NC(CC(=O)O)C(=O)NC(CCCCN)C(=O)NC(CC(=O)O)C(=O)NC(CSC1CC(=O)N(CCCCCC(=O)NC(C(=O)N2CCCC2C(=O)Nc2ccc(COC(*)=O)cc2)C(C)C)C1=O)C(=O)O ; *NCCCCCN1C(=O)CC(SCC(NC(=O)C(CC(=O)O)NC(=O)C(CCCCN)NC(=O)C(CC(=O)O)N*)C(=O)O)C1=O]

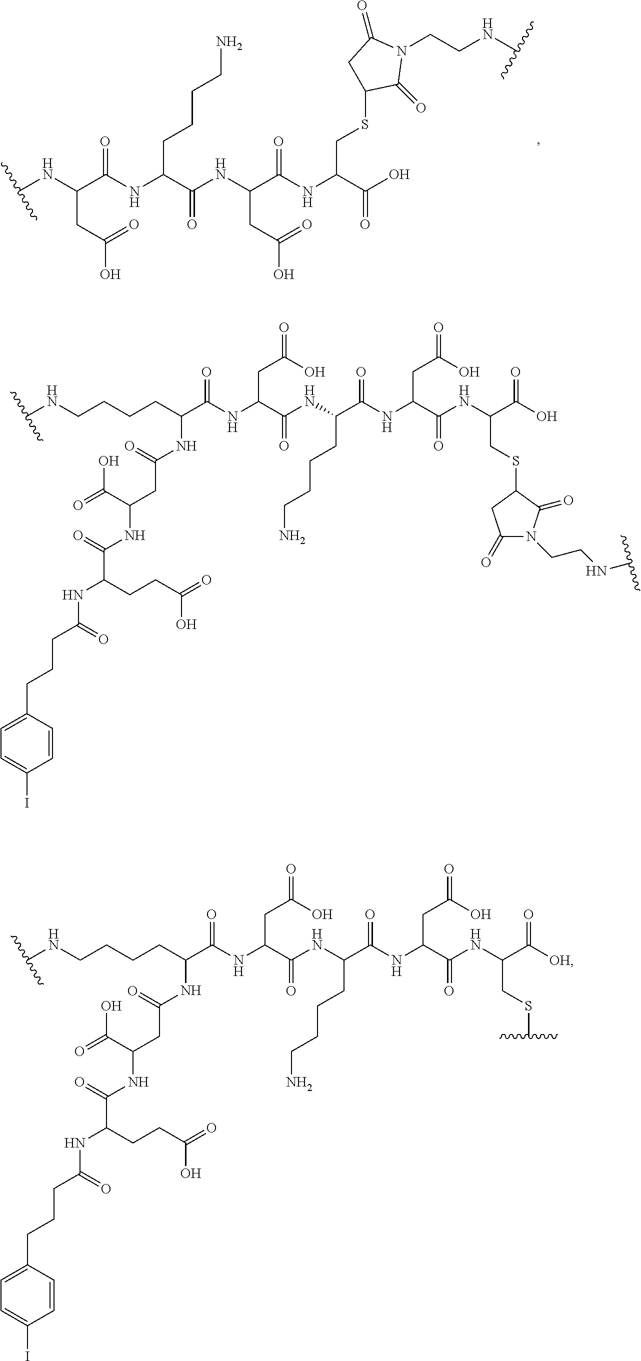 [SMILES: *NCCN1C(=O)CC(SCC(NC(=O)C(CC(=O)O)NC(=O)C(CCCCN)NC(=O)C(CC(=O)O)N*)C(=O)O)C1=O ; *NCCCCC(NC(=O)CC(NC(=O)C(CCC(=O)O)NC(=O)CCCc1ccc(I)cc1)C(=O)O)C(=O)NC(CC(=O)O)C(=O)N[C@@H](CCCCN)C(=O)NC(CC(=O)O)C(=O)NC(CSC1CC(=O)N(CCN*)C1=O)C(=O)O ; *NCCCCC(NC(=O)CC(NC(=O)C(CCC(=O)O)NC(=O)CCCc1ccc(I)cc1)C(=O)O)C(=O)NC(CC(=O)O)C(=O)NC(CCCCN)C(=O)NC(CC(=O)O)C(=O)NC(CS*)C(=O)O]

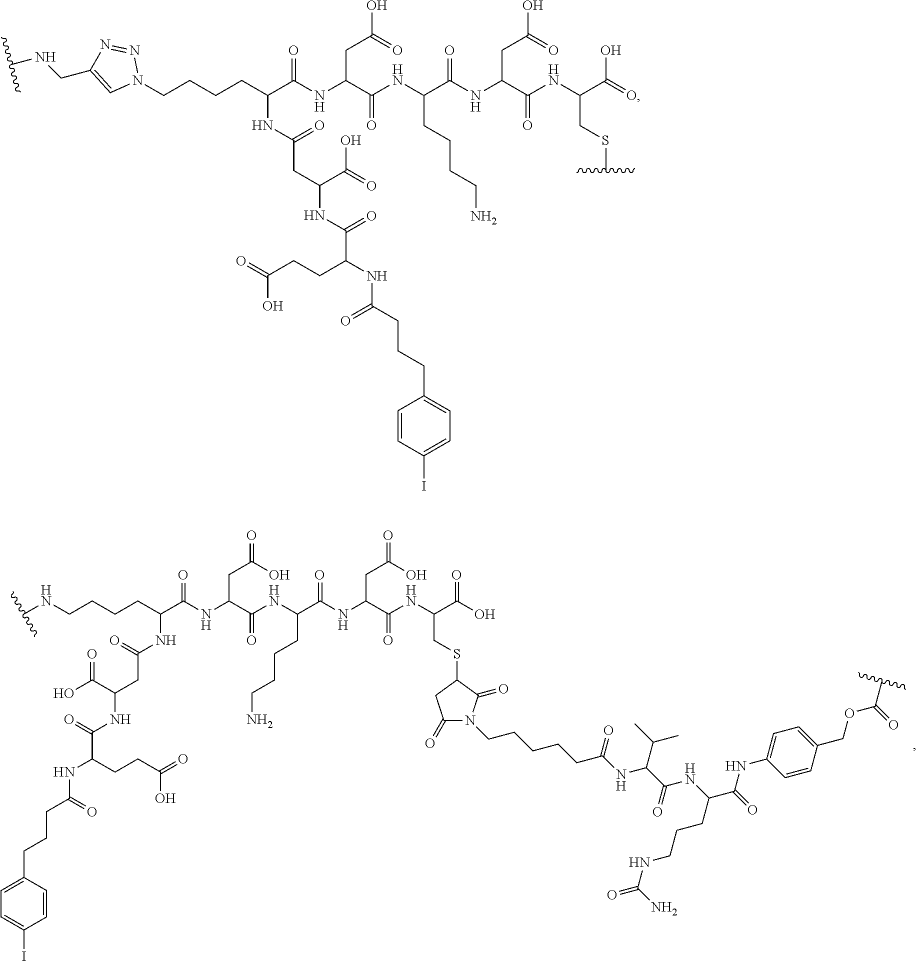 [SMILES: *NCc1cn(CCCCC(NC(=O)CC(NC(=O)C(CCC(=O)O)NC(=O)CCCc2ccc(I)cc2)C(=O)O)C(=O)NC(CC(=O)O)C(=O)NC(CCCCN)C(=O)NC(CC(=O)O)C(=O)NC(CS*)C(=O)O)nn1 ; *NCCCCC(NC(=O)CC(NC(=O)C(CCC(=O)O)NC(=O)CCCc1ccc(I)cc1)C(=O)O)C(=O)NC(CC(=O)O)C(=O)NC(CCCCN)C(=O)NC(CC(=O)O)C(=O)NC(CSC1CC(=O)N(CCCCCC(=O)NC(C(=O)NC(CCCNC(N)=O)C(=O)Nc2ccc(COC(*)=O)cc2)C(C)C)C1=O)C(=O)O]

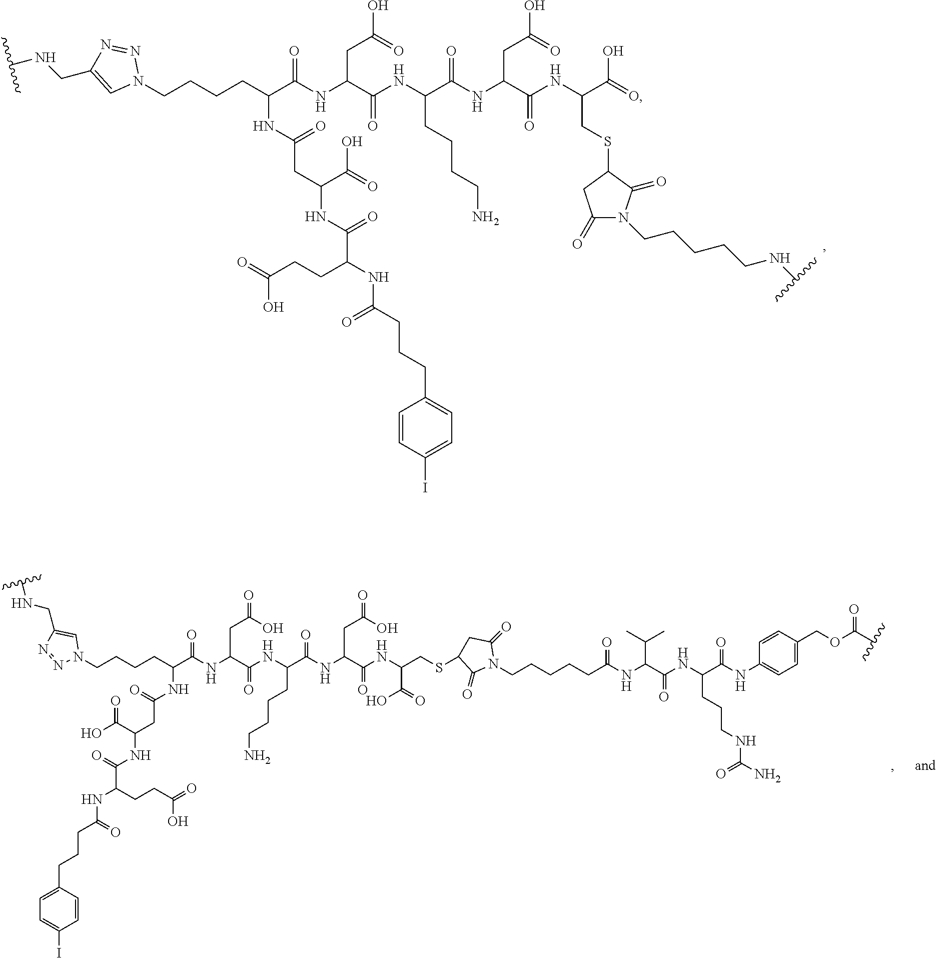 [SMILES: *NCCCCCN1C(=O)CC(SCC(NC(=O)C(CC(=O)O)NC(=O)C(CCCCN)NC(=O)C(CC(=O)O)NC(=O)C(CCCCn2cc(CN*)nn2)NC(=O)CC(NC(=O)C(CCC(=O)O)NC(=O)CCCc2ccc(I)cc2)C(=O)O)C(=O)O)C1=O ; *NCc1cn(CCCCC(NC(=O)CC(NC(=O)C(CCC(=O)O)NC(=O)CCCc2ccc(I)cc2)C(=O)O)C(=O)NC(CC(=O)O)C(=O)NC(CCCCN)C(=O)NC(CC(=O)O)C(=O)NC(CSC2CC(=O)N(CCCCCC(=O)NC(C(=O)NC(CCCNC(N)=O)C(=O)Nc3ccc(COC(*)=O)cc3)C(C)C)C2=O)C(=O)O)nn1]

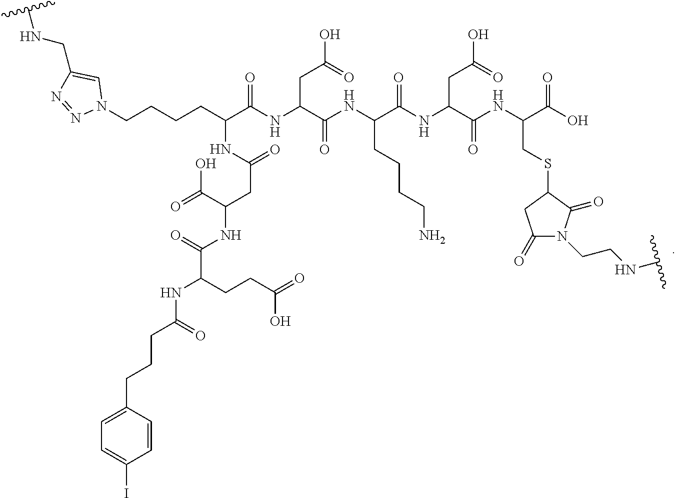 [SMILES: *NCCN1C(=O)CC(SCC(NC(=O)C(CC(=O)O)NC(=O)C(CCCCN)NC(=O)C(CC(=O)O)NC(=O)C(CCCCn2cc(CN*)nn2)NC(=O)CC(NC(=O)C(CCC(=O)O)NC(=O)CCCc2ccc(I)cc2)C(=O)O)C(=O)O)C1=O]

28.-30. (canceled)

31. The compound according to claim 26, wherein:
(a) A or 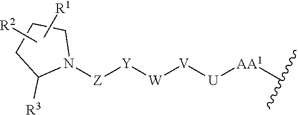
is represented by
A²: 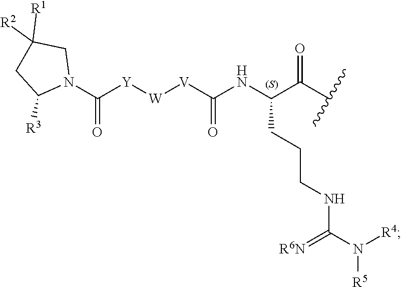
at least one of R⁴, R⁵ and R⁶ is not H; and
W is 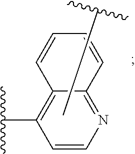
or
(b) A or 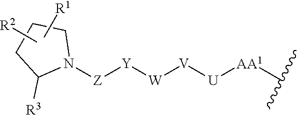
is represented by
A^{2′c}:

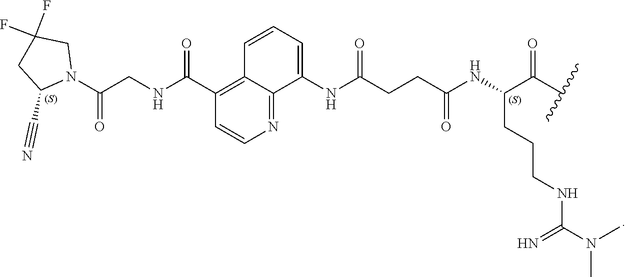

32. (canceled)

33. The compound according to claim 27, which has one of the following structures, wherein -D represents —B—C: 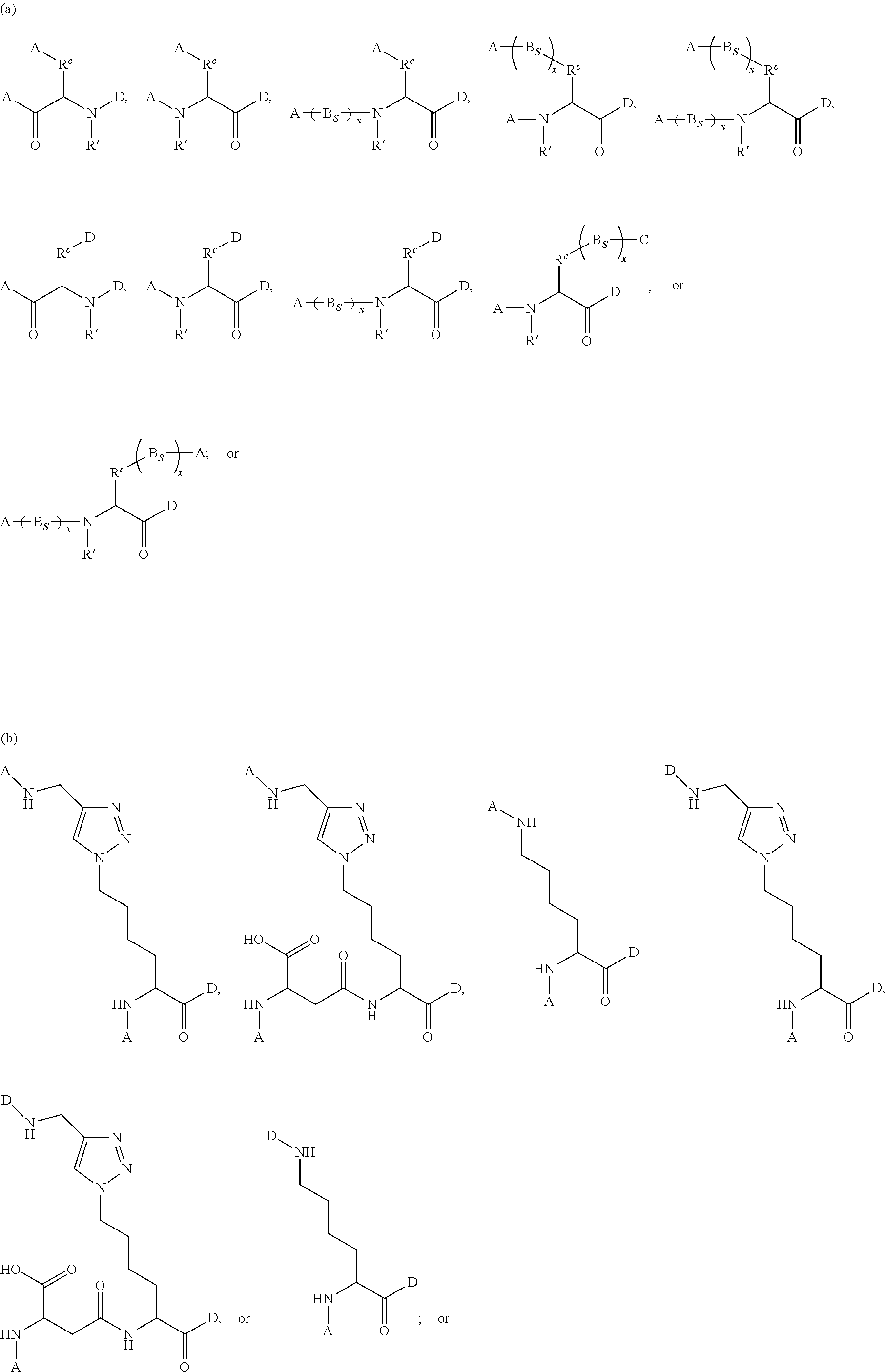 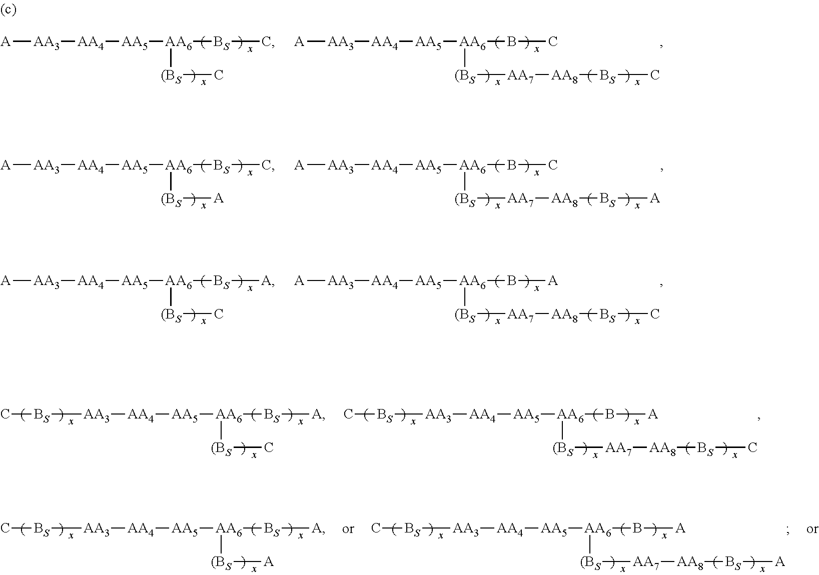 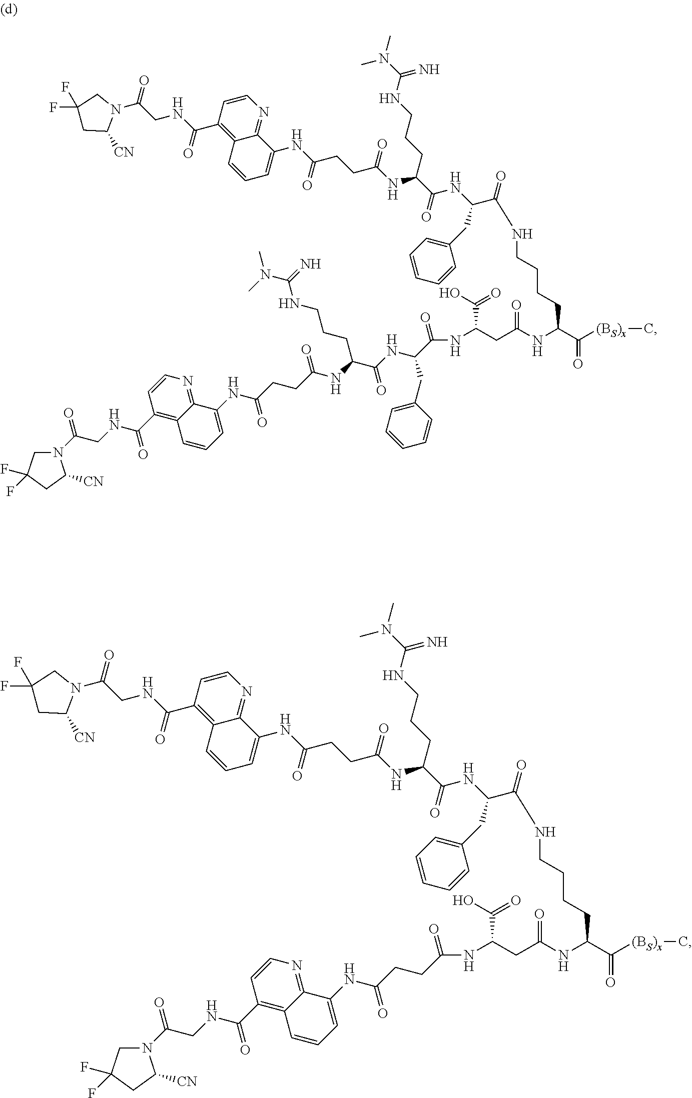 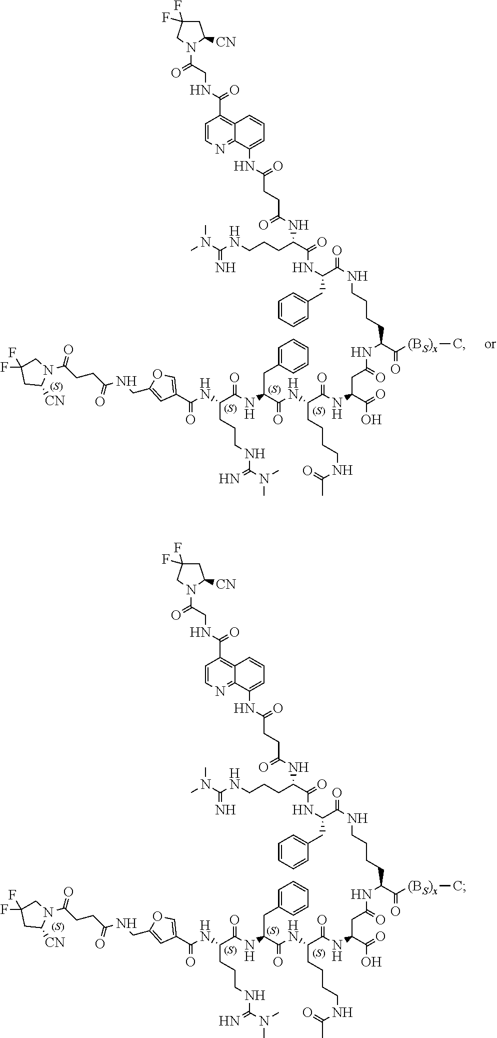
wherein, unless otherwise specified, all groups and variables are defined hereinabove,
each R^{c}, is independently selected from: H; optionally substituted C₁₋₆ alkyl; (C₃-C₁₀ carbocyclyl)C₁₋₆ alkyl; (C₆-C₁₀ aryl)C₁₋₆ alkyl; (C₁-C₁₀ heterocyclyl)C₁₋₆ alkyl; C₂₋₆ alkenyl; C₂₋₆ alkynyl; C₆-C₁₀ aryl, in each of which optionally one or more of the carbon atoms can be replaced by heteroatoms; and is side-chain residues of proteinogenic or a non-proteinogenic amino acids;
each R′ is independently H or selected from: H; SH; NH₂; halogen; cyano; carboxy; C₁₋₆-alkyl; O(C₁₋₆ alkyl); S(C₁₋₆-alkyl); C₂₋₆ alkenyl; C₂₋₆ alkynyl; C₁₋₆ heteroalkenyl; C₁₋₆ heteroalkynyl; C₃₋₁₀ cycloalkenyl; C₁₋₁₀ cycloheteroalkenyl; C₆₋₁₀ aryl; and (C₆₋₁₀ aryl)C₁₋₆ alkyl, each of which being optionally substituted with from 1 to 3 substituents selected from —OH, oxo and halo, and optionally connected to an atom in Z, Y, W or V;
and each x is 0, 1, 2, 3, 4, 5, 6, 7, 8, 9, or 10; or 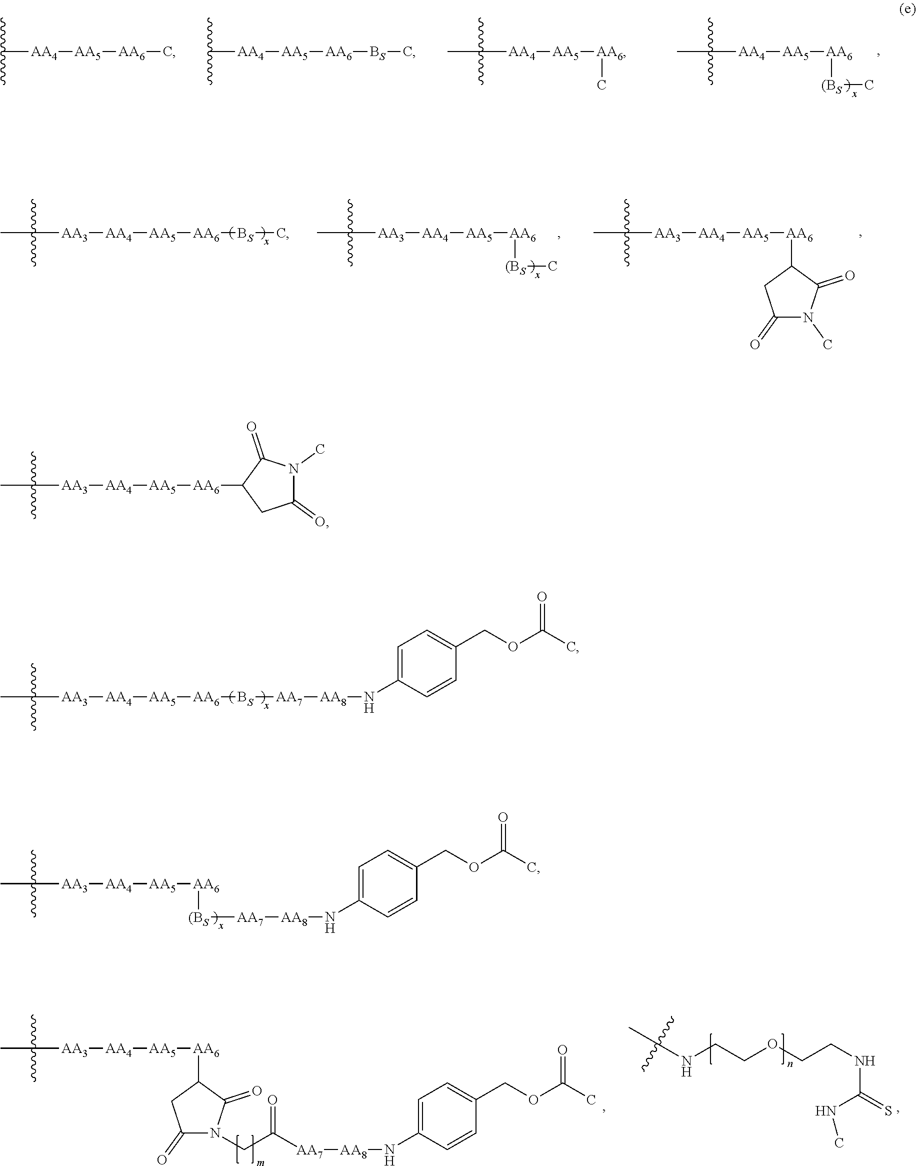 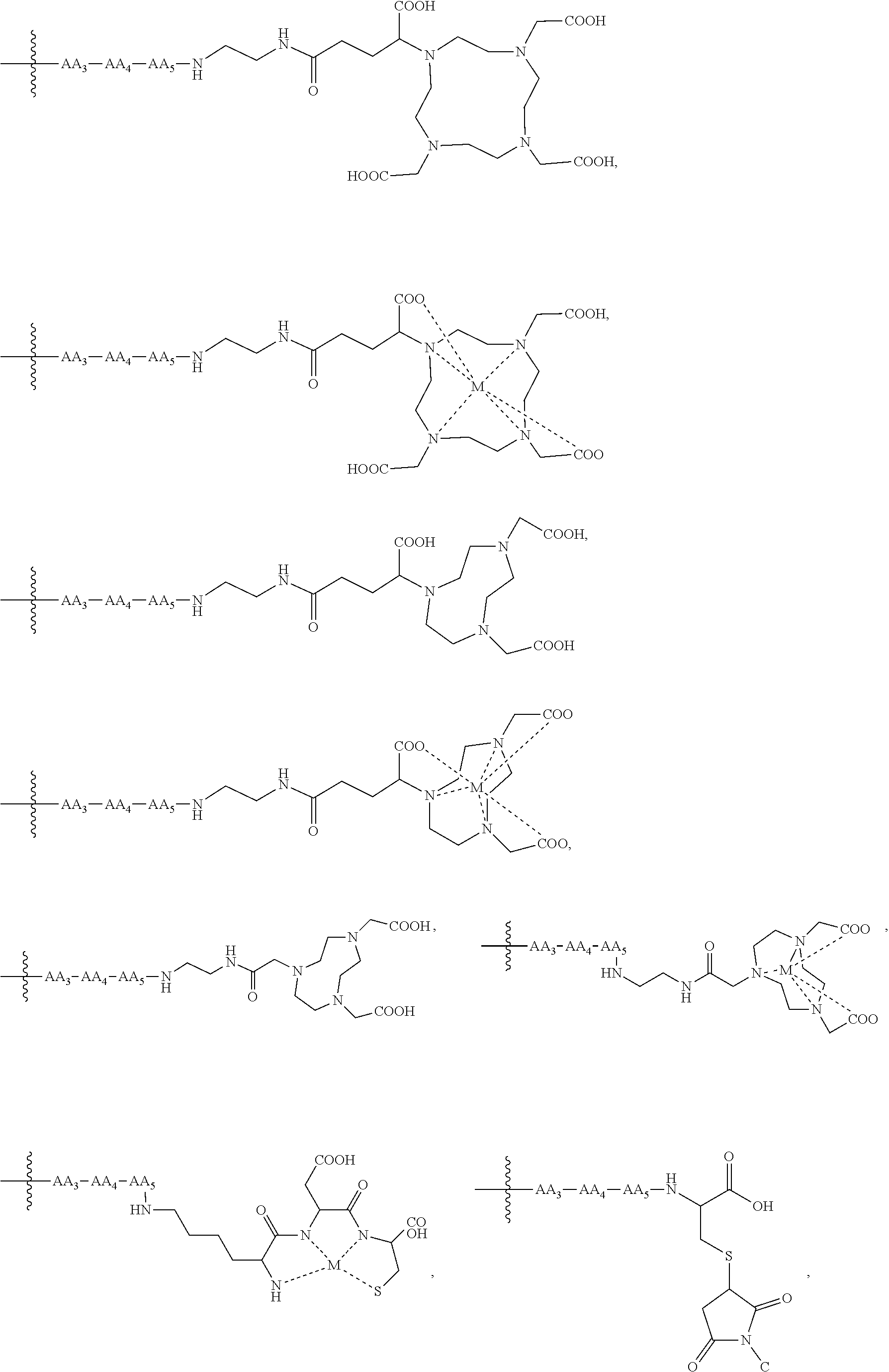 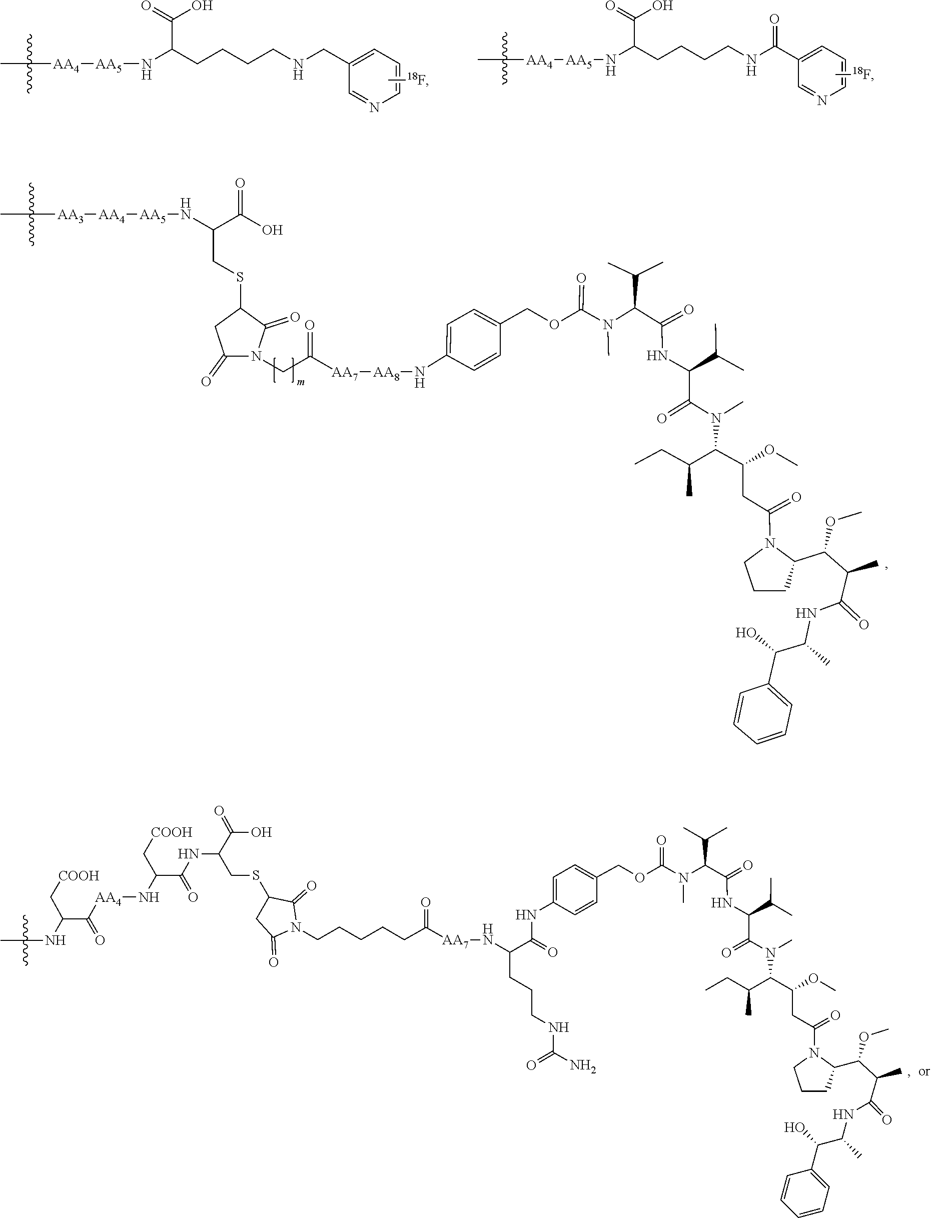 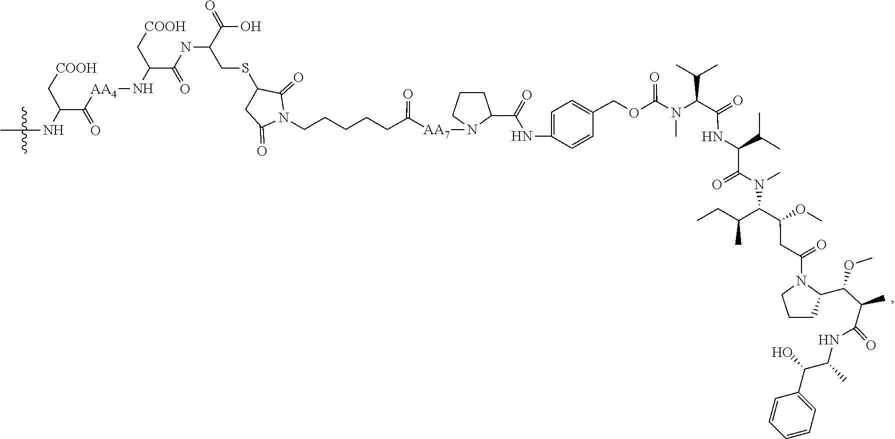
each x is 0, 1, 2, 3, 4, 5, 6, 7, 8, 9, or 10;
each AA₃, AA₄, AA₅, AA₆, AA and AA₈ is independently is 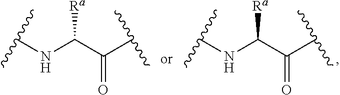
wherein R^{a} is —CH₃, —CH(CH₃)₂, —CH₂C≡CH, —((CH)₂)₄NH₂ or —CH₂-Ph.

34. (canceled)

35. The compound according to claim 25, wherein C is a chelating agent group suitable for radiolabeling; a radioactive group comprising a radioisotope; a chelate of a radioactive isotope with a chelating agent; a fluorophore group; a cytotoxic and/or cytostatic agent; immunomodulator agent; or a protein.

36. The compound according to claim 35, wherein:
(a) the chelating agent group suitable for radiolabeling is selected from: sulfur colloid, diethylenetriaminepentaacetic acid (DTPA), ethylenediaminetetraacetic acid (EDTA), 1,4,7,10-tetraazacyclododecane-N,N′,N″,N″′-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA), iminodiacetic acid, bis(carboxymethylimidazole)glycine, 6-Hydrazinopyridine-3-carboxylic acid (HYNIC), 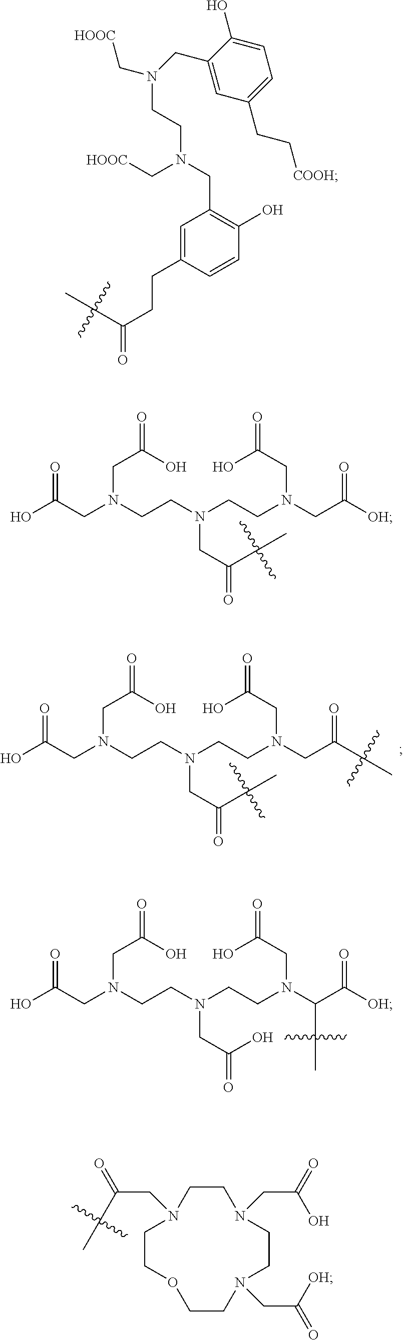 [SMILES: *C(=O)CCc1ccc(O)c(CN(CCN(CC(=O)O)Cc2cc(CCC(=O)O)ccc2O)CC(=O)O)c1 ; *C(=O)CN(CCN(CC(=O)O)CC(=O)O)CCN(CC(=O)O)CC(=O)O ; *C(=O)CN(CCN(CC(*)=O)CC(=O)O)CCN(CC(=O)O)CC(=O)O ; *C(C(=O)O)N(CCN(CCN(CC(=O)O)CC(=O)O)CC(=O)O)CC(=O)O ; *C(=O)CN1CCOCCN(CC(=O)O)CCN(CC(=O)O)CC1] 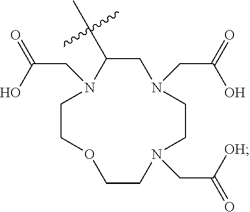 [SMILES: *C1CN(CC(=O)O)CCN(CC(=O)O)CCOCCN1CC(=O)O] 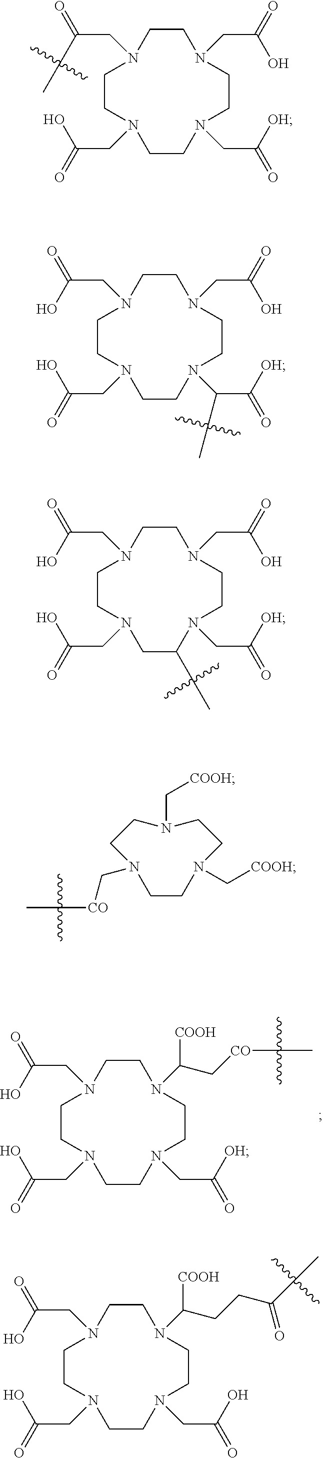 [SMILES: *C(=O)CN1CCN(CC(=O)O)CCN(CC(=O)O)CCN(CC(=O)O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O ; *C(=O)CN1CCN(CC(=O)O)CCN(CC(=O)O)CC1 ; *C(=O)CC(C(=O)O)N1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1] 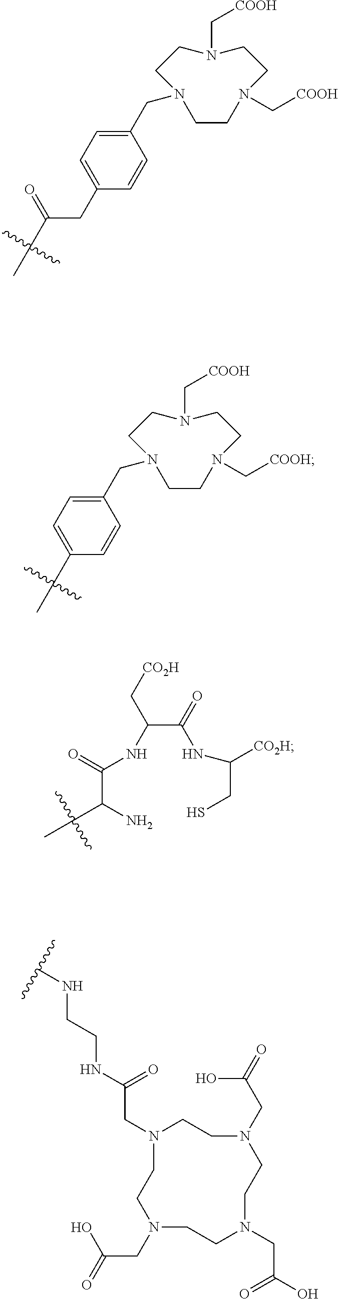 [SMILES: *C(=O)Cc1ccc(CN2CCN(CC(=O)O)CCN(CC(=O)O)CC2)cc1 ; *c1ccc(CN2CCN(CC(=O)O)CCN(CC(=O)O)CC2)cc1 ; *C(N)C(=O)NC(CC(=O)O)C(=O)NC(CS)C(=O)O ; *NCCNC(=O)CN1CCN(CC(=O)O)CCN(CC(=O)O)CCN(CC(=O)O)CC1] 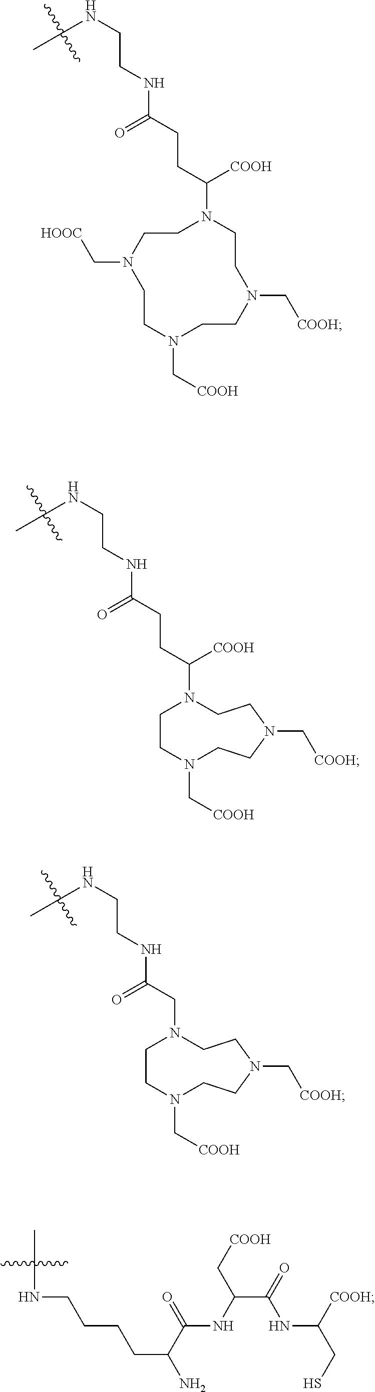 [SMILES: *NCCNC(=O)CCC(C(=O)O)N1CCN(CC(=O)O)CCN(CC(=O)O)CCN(CC(=O)O)CC1 ; *NCCNC(=O)CCC(C(=O)O)N1CCN(CC(=O)O)CCN(CC(=O)O)CC1 ; *NCCNC(=O)CN1CCN(CC(=O)O)CCN(CC(=O)O)CC1 ; *NCCCCC(N)C(=O)NC(CC(=O)O)C(=O)NC(CS)C(=O)O]
has a structure according to the following formula: 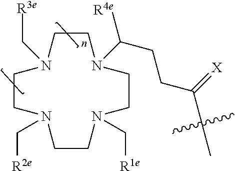
wherein:
n is 0, 1, 2, 3, 4 or 5;
R^{1e} is independently H, COOH, aryl-COOH or heteroaryl-COOH;
R^{2e} is independently H, COOH, aryl-COOH or heteroaryl-COOH;
each R^{3e} is independently H, COOH, aryl-COOH or heteroaryl-COOH;
R^{4e} is independently H, COOH, aryl-COOH or heteroaryl-COOH; and
X is O, NH or S; or
has a structure according to the following formula: 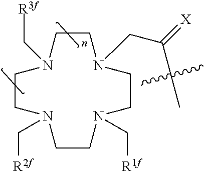
wherein:
n is 0, 1, 2, 3, 4 or 5;
R^{1f} is independently H, COOH, aryl-COOH or heteroaryl-COOH;
R^{2f} is independently H, COOH, aryl-COOH or heteroaryl-COOH;
R^{3f} is independently H, COOH, aryl-COOH or heteroaryl-COOH; and
X is O, NH or S;
(b) the radioactive group comprising a radioisotope is selected from: ²²³Ra, ⁸⁹Sr, ^{94m}Tc, ^{99m}Tc, ¹⁸⁶Re, ¹⁸⁸Re, ²⁰³Pb, ⁶⁷Ga, ⁶⁸Ga, ⁴⁷Sc, 11In, ⁹⁷Ru, ⁶²Cu, ⁶⁴Cu, ⁸⁶Y, ⁸⁸Y, ⁹⁰Y, ¹²¹Sn, ¹⁶¹Tb, ¹⁵³Sm, ¹⁶⁶Ho, ¹⁰⁵Rh, ¹⁷⁷Lu, ¹²³I, ¹²⁴I, ¹²⁵I, ¹³¹I, ¹⁸F, ²¹¹At, ²²⁵Ac, ⁸⁹Sr, ²²⁵Ac, ^{117m}Sn and ¹⁶⁹Er;
(c) the chelate of a radioactive isotope is a chelate of an isotope listed under (b) above and/or with a chelating agent listed under (a) above;
(d) moiety C is a group selected from any of the following structures: 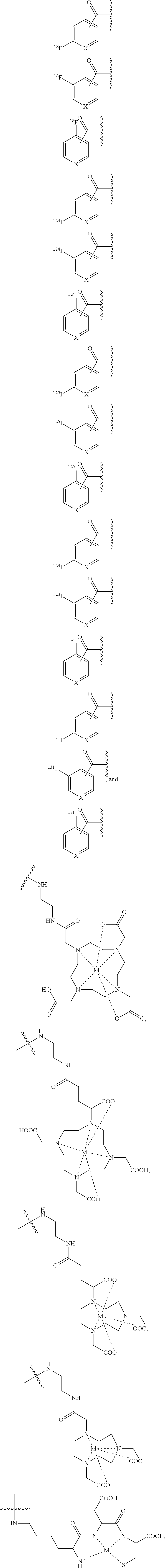
wherein M is a radioactive isotope, or wherein M is a radioactive isotope selected among the list under (b) above; or
(e) wherein C is a group selected from any of the following structures: 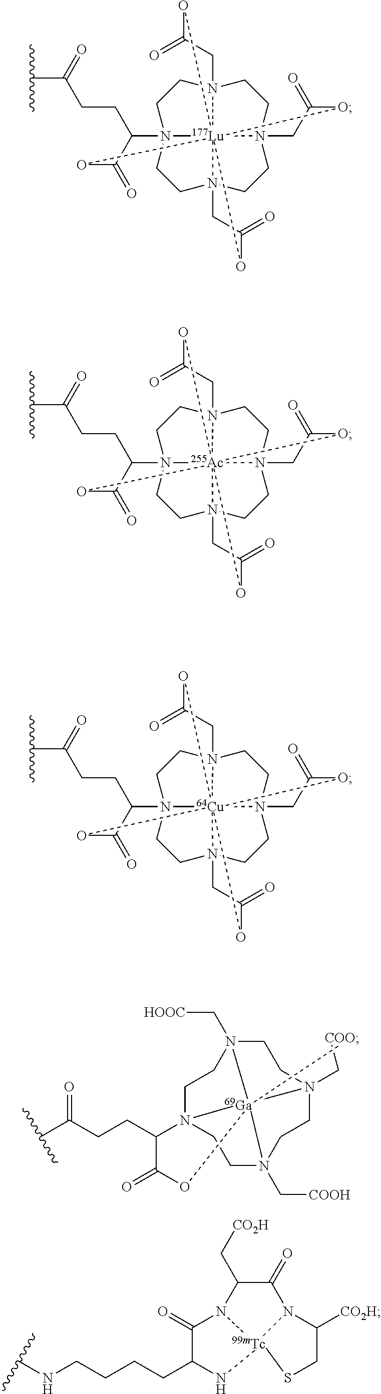 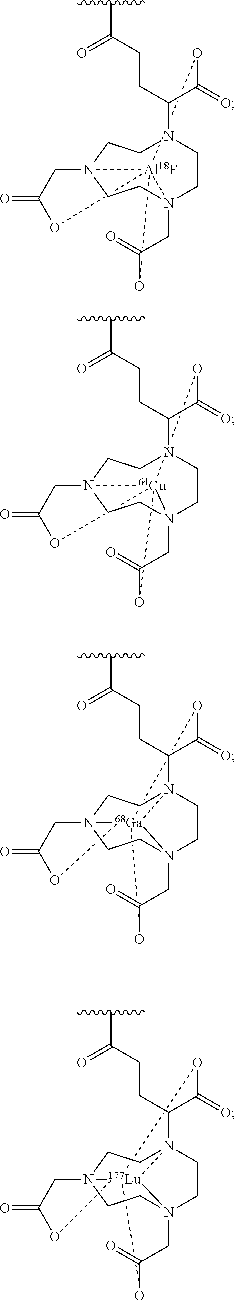 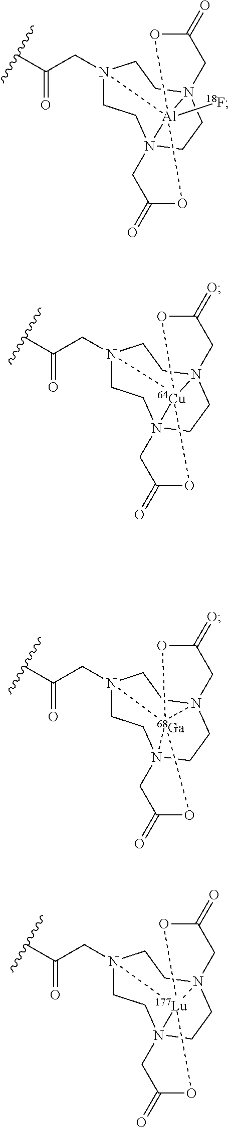 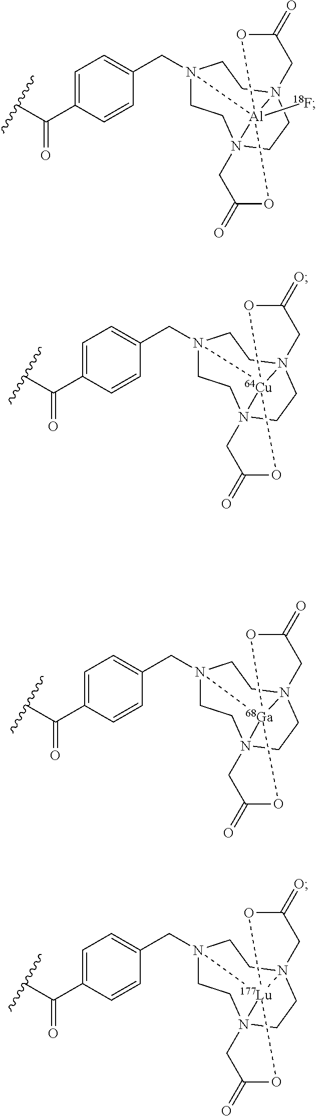
or
(f) the fluorophore group is selected from: a xanthene dye, acridine dye, oxazine dye, cyanine dye, styryl dye, coumarine dye, porphine dye, fluorescent metal-ligand-complex, fluorescent protein, nanocrystals, perylene dye, boron-dipyrromethene dye and phtalocyanine dye; or the following structures: 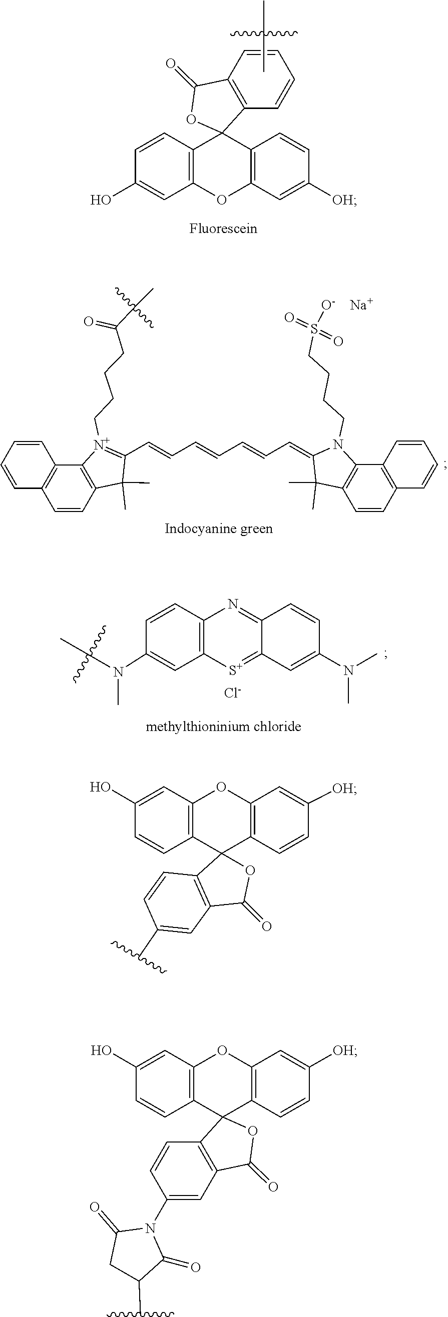 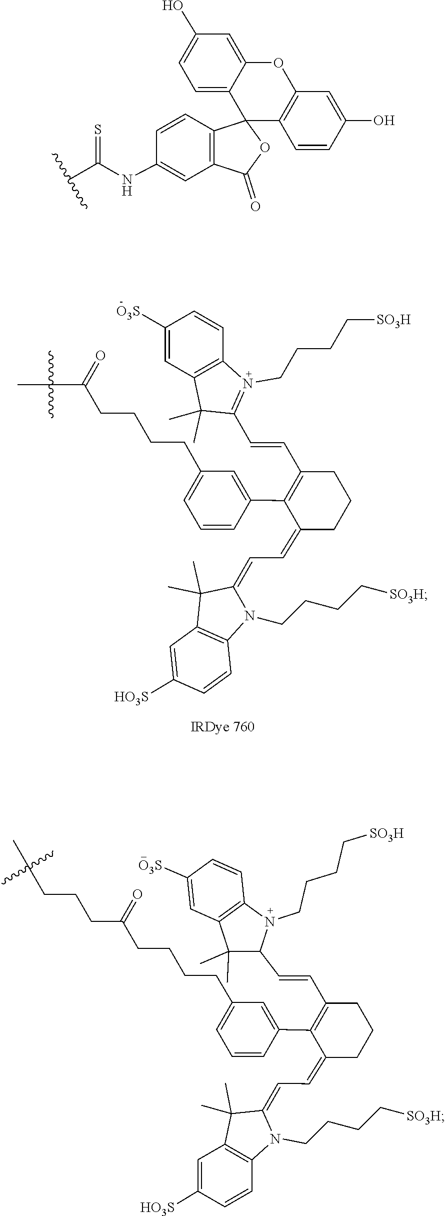 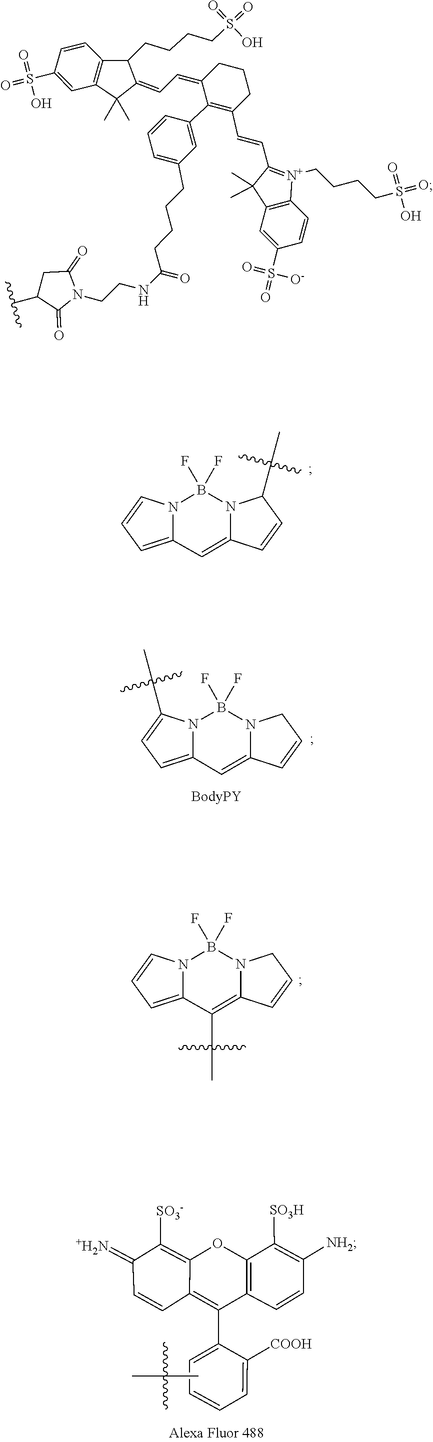 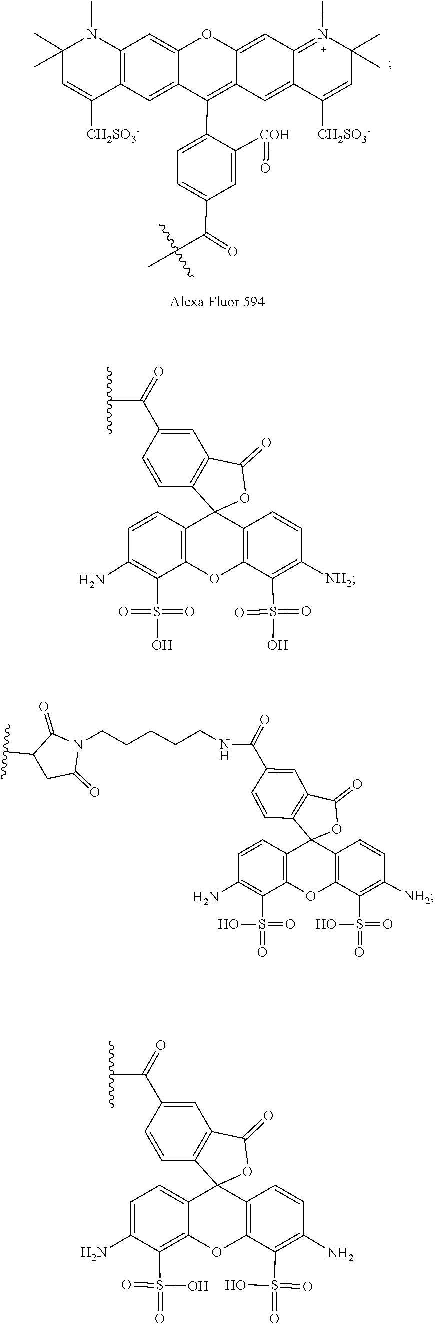
(g) the cytotoxic and/or cytostatic agent is selected from: chemotherapeutic agent selected from the group consisting of topoisomerase inhibitors, alkylating agents, antimetabolites, antibiotics, mitotic disrupters, DNA intercalating agents, DNA synthesis inhibitors, DNA-RNA transcription regulator, enzyme inhibitors, gene regulators, hormone response modifiers, hypoxia-selective cytotoxins, epidermal growth factor inhibitors, anti-vascular agents and a combination of two or more thereof; or from the following structures: 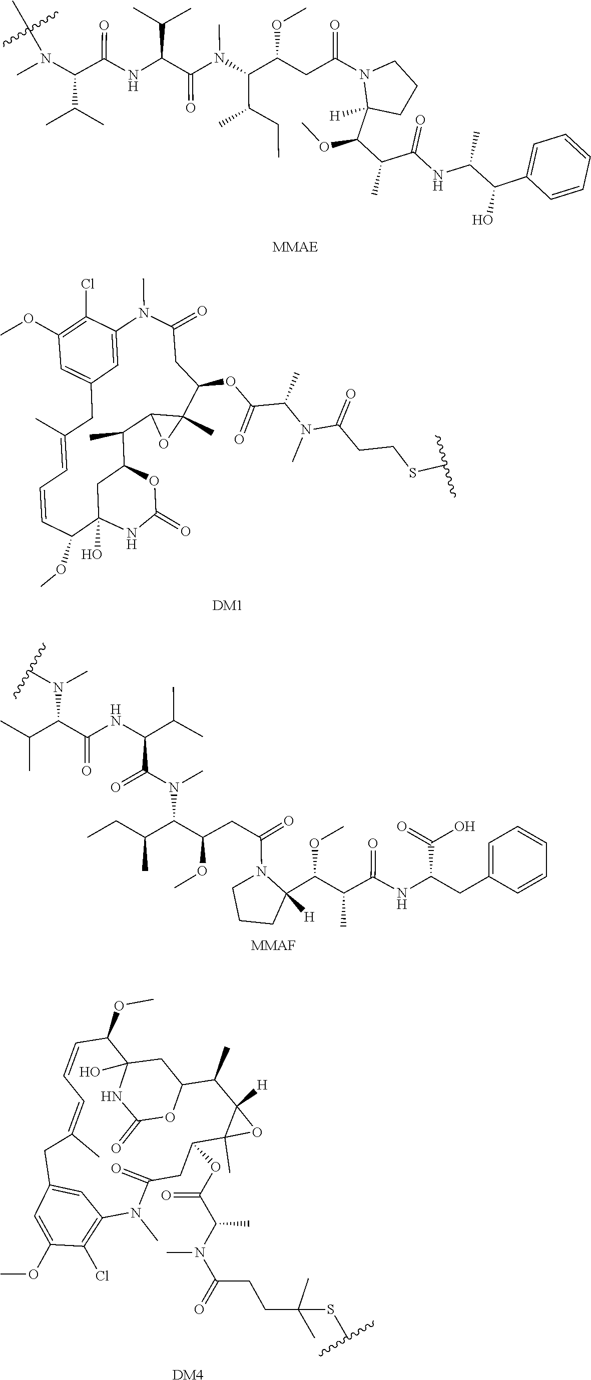 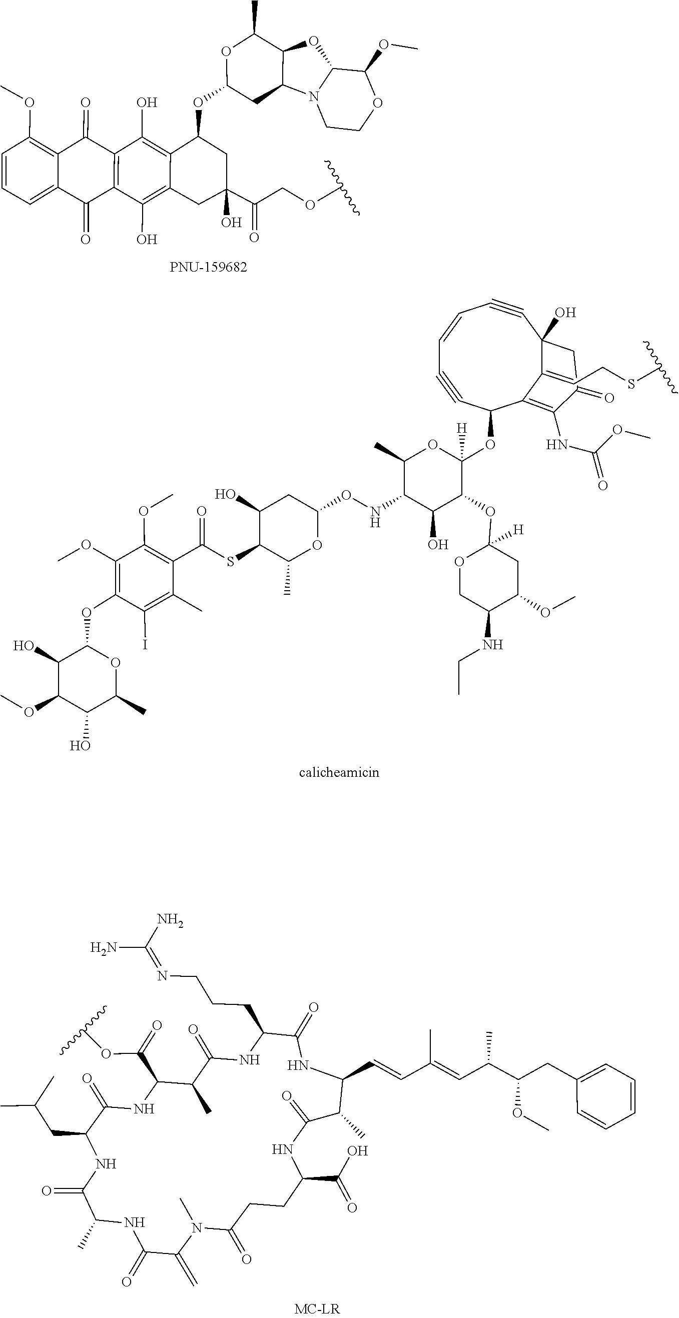 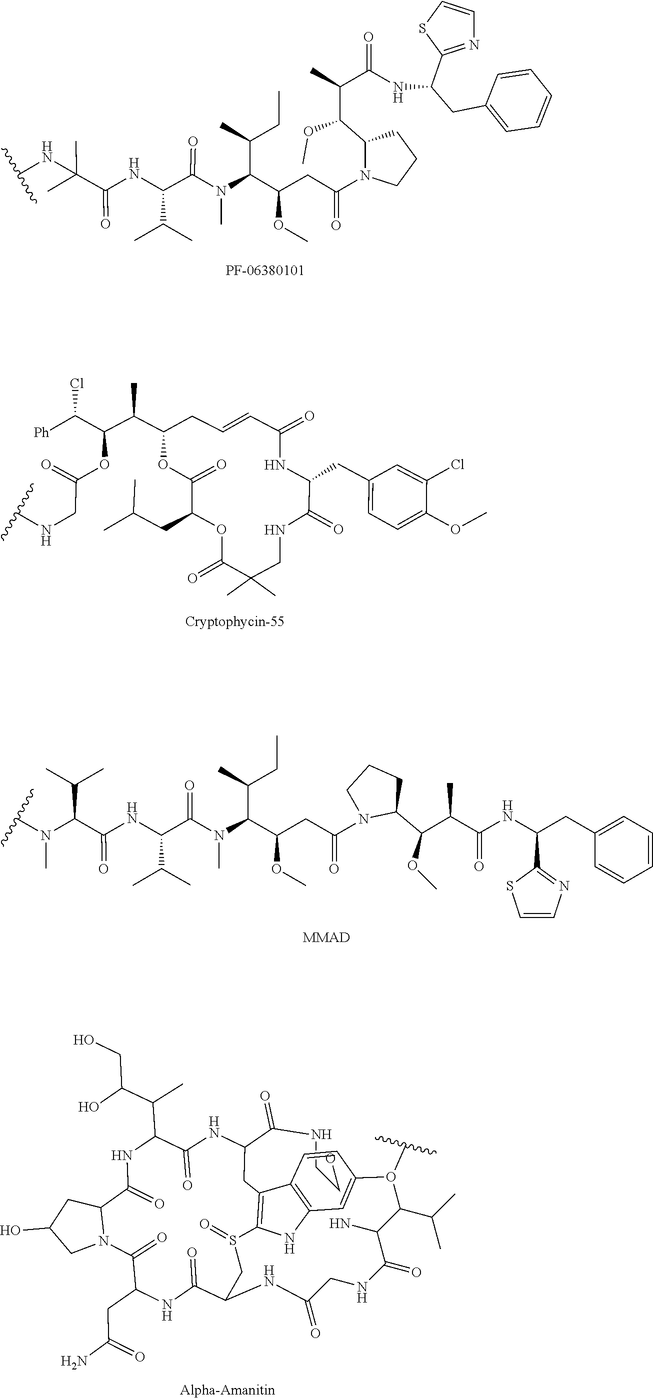 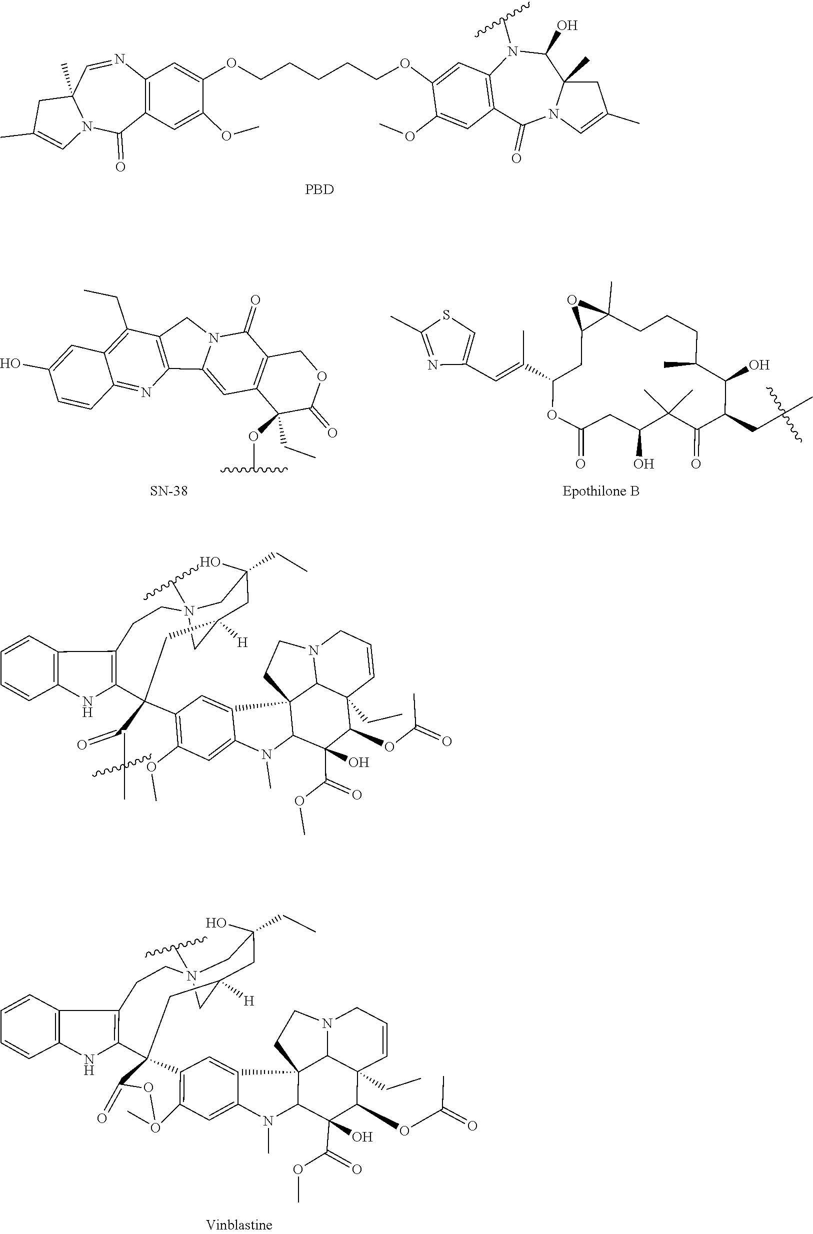 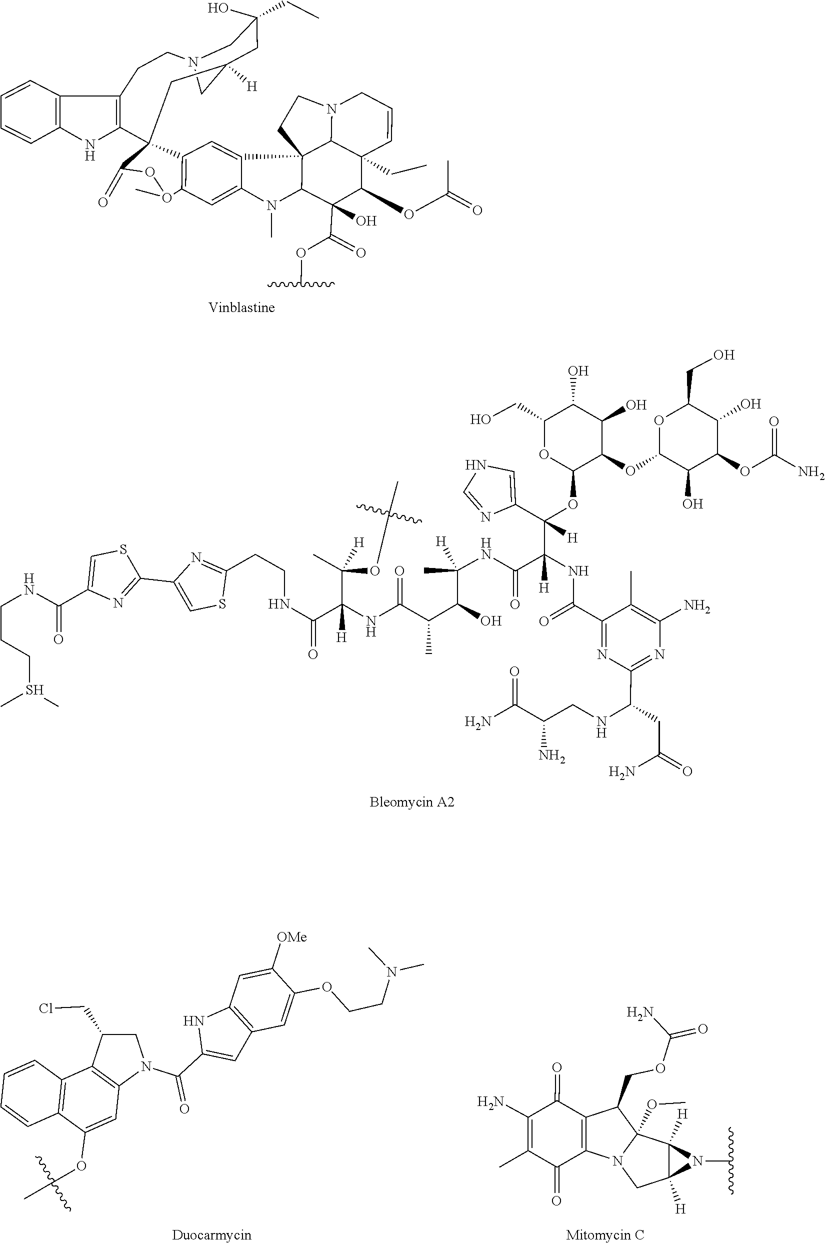 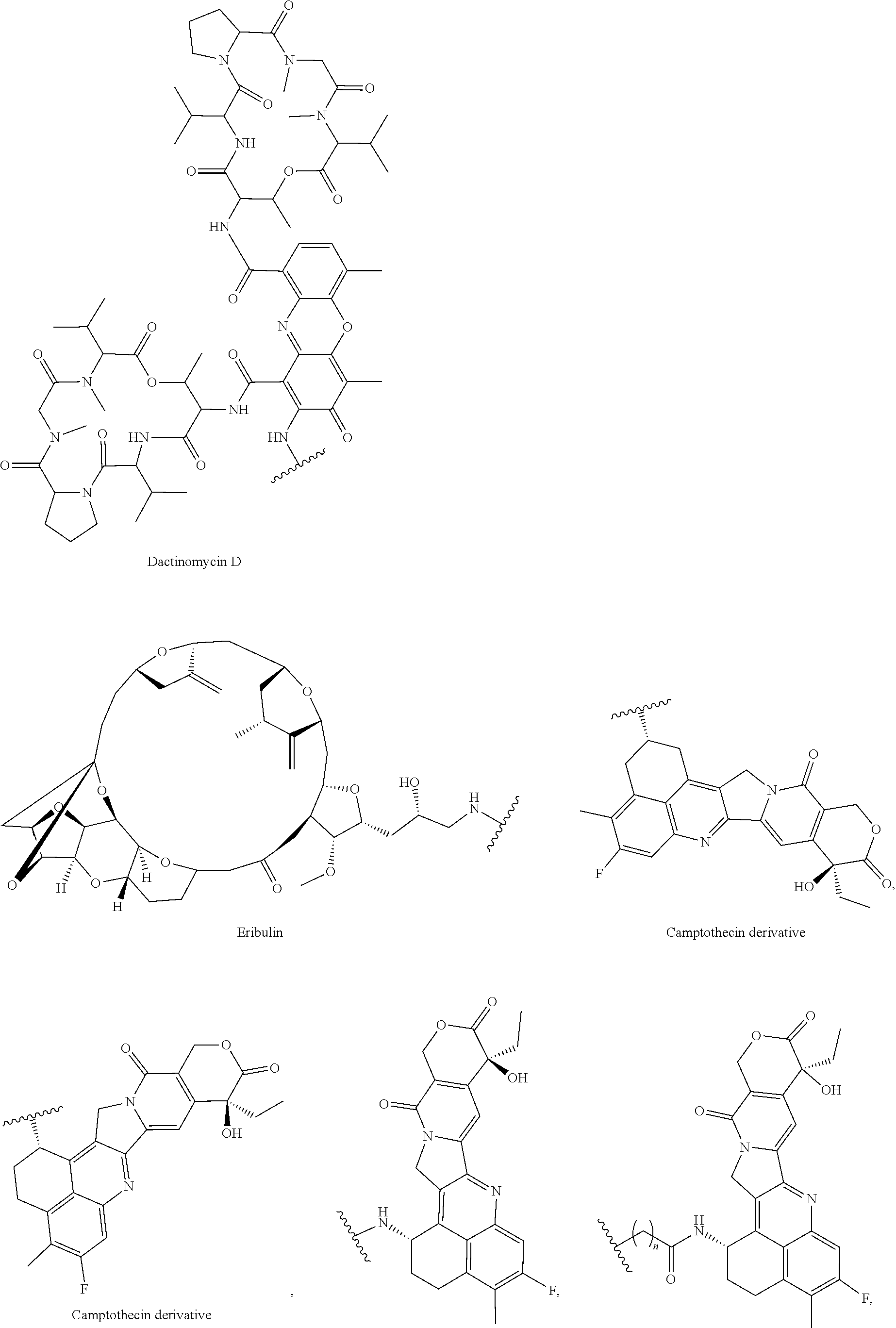 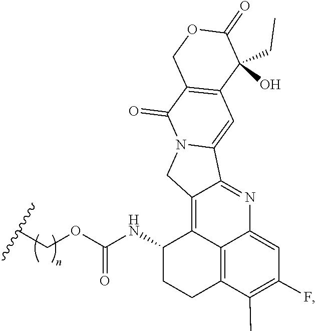
wherein each n is 0, 1, 2, 3, 4, 5 or 6; 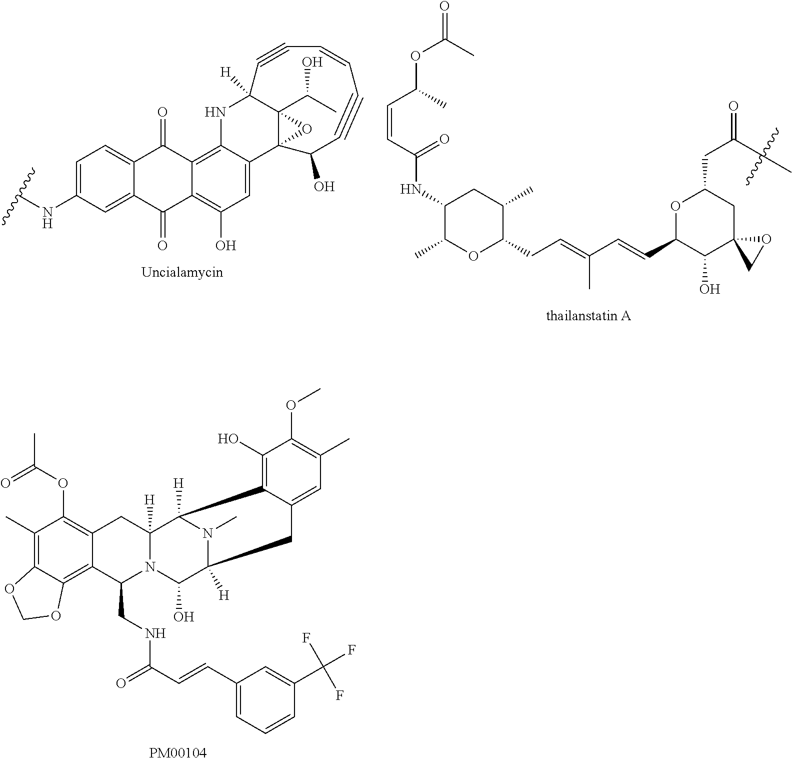 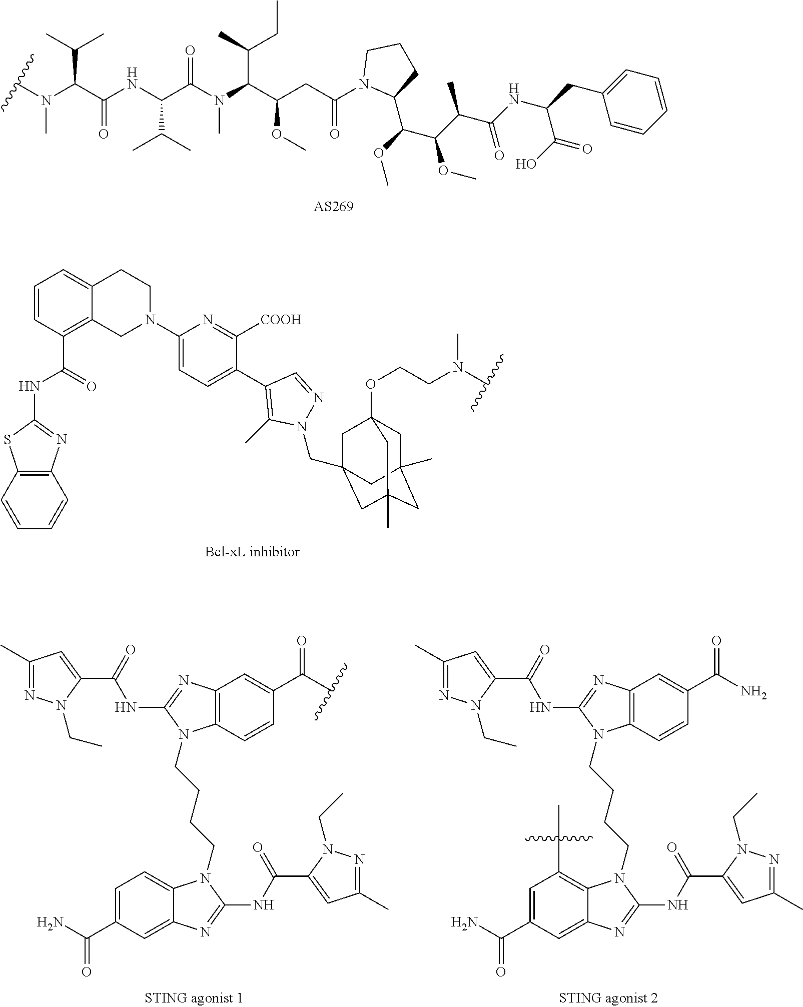 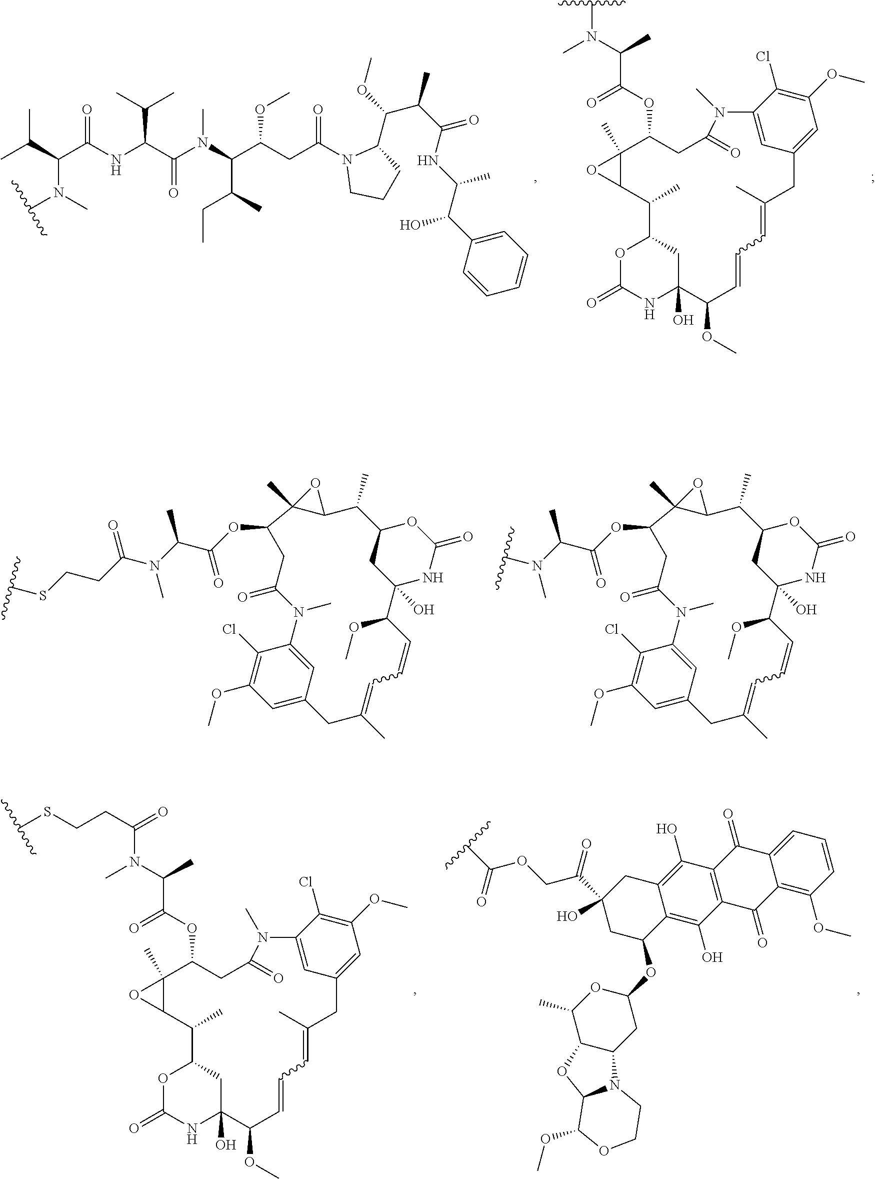 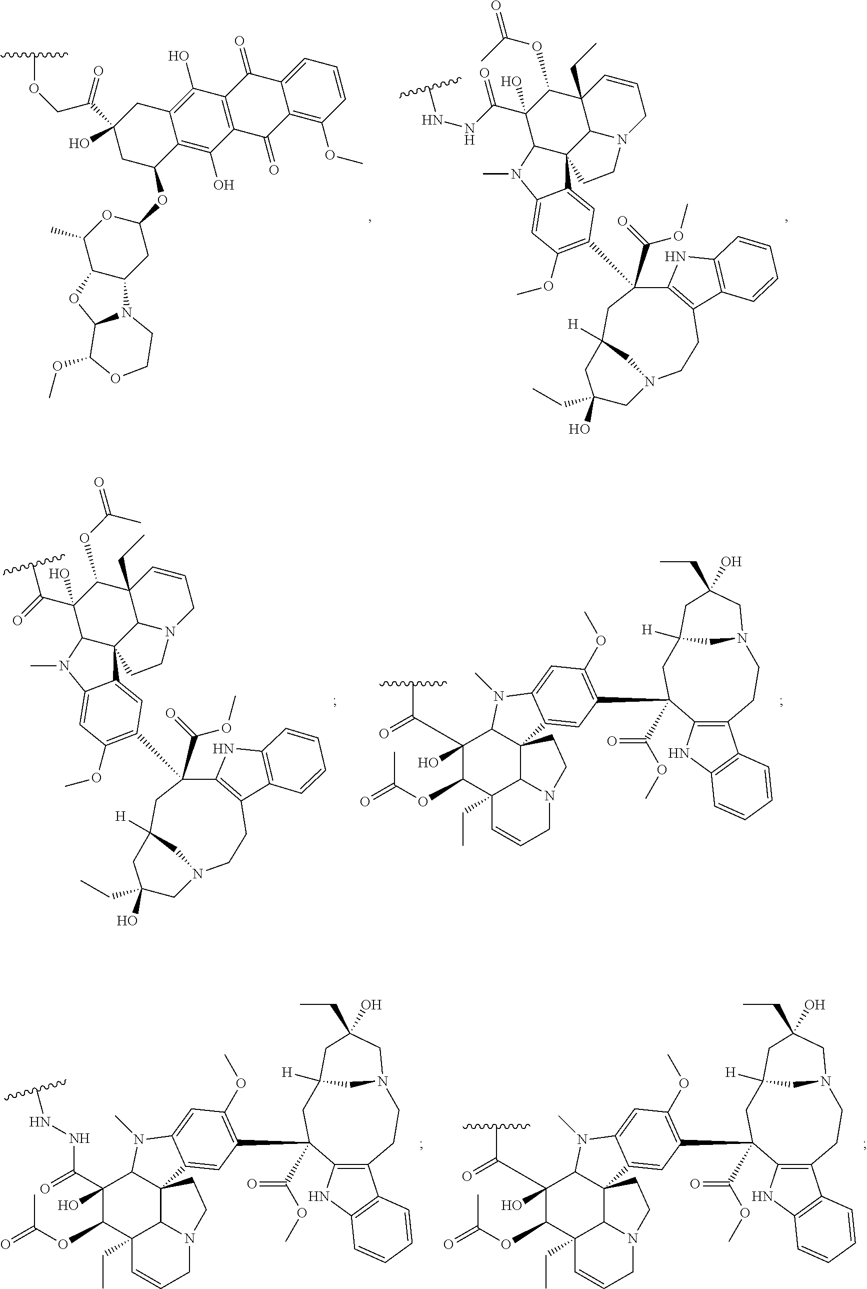 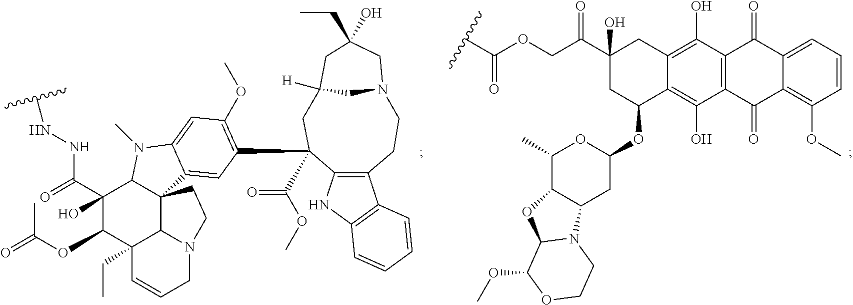
moiety C is an auristatin; or
moiety C has a structure according to the following formula: 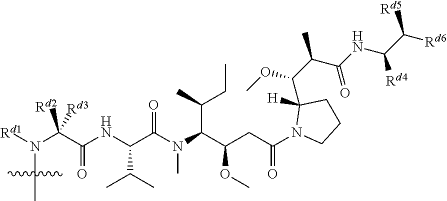
wherein:
R^{1d} is independently H, C₁-C₆ alkyl; or CH₃;
R^{2d} is independently C₁-C₆ alkyl; CH₃ or iPr;
R^{3d} is independently H, C₁-C₆ alkyl; or CH₃;
R^{4d} is independently H, C₁-C₆ alkyl, COO(C₁-C₆ alkyl), CON(H or C₁-C₆ alkyl), C₃-C₁₀ aryl, C₃-C₁₀ heteroaryl; CH₃, COOH, COOCH₃ or thiazolyl;
R^{5d} is independently H, OH, C₁-C₆ alkyl; and
R^{6d} is independently optionally substituted C₃-C₁₀ aryl or C₃-C₁₀ heteroaryl; or optionally substituted phenyl or pyridyl;
or wherein moiety C is derived from MMAE or MMAF; or
(h) the immunomodulator agent is selected from: molecules known to be able to modulate the immune system, ligands of CD3, CD25, TLRs, STING, 4-1BBL, 4-1BB, PD-1, mTor, PDL-1, NKG-2D IMiDs, wherein ligands can be agonists and/or antagonist; or
(i) the protein is selected from cytokines, such as IL2, IL10, IL12, IL15, TNF, Interferon Gamma, or is an antibody.

37. The compound according to claim 27, having one of the following structures:

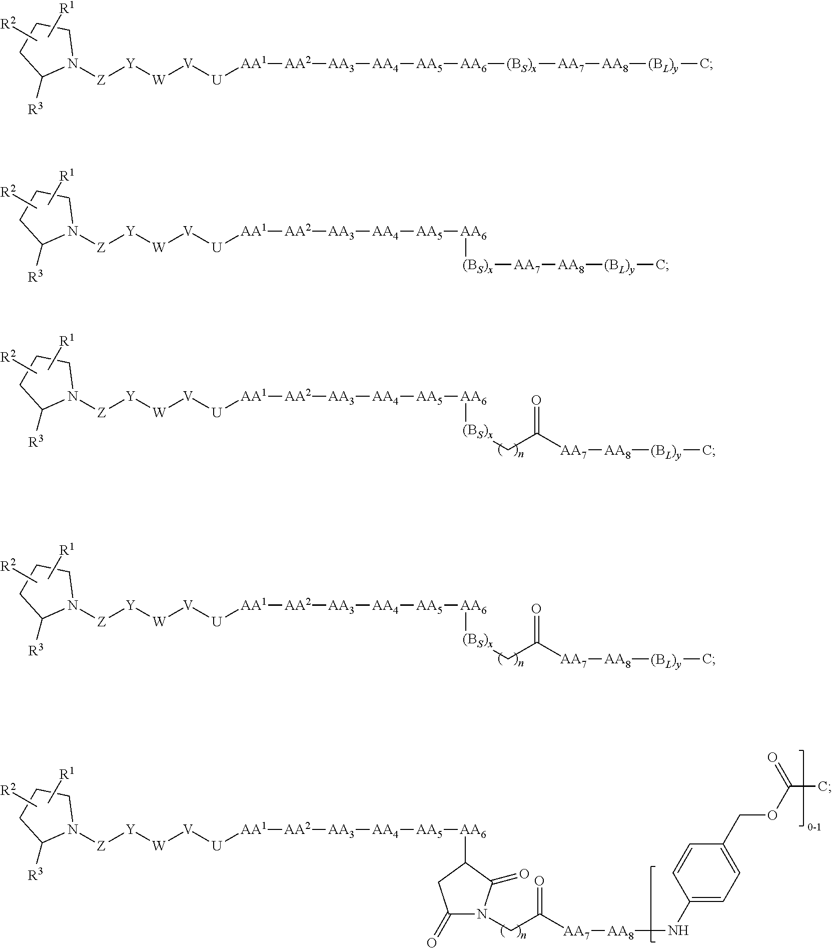

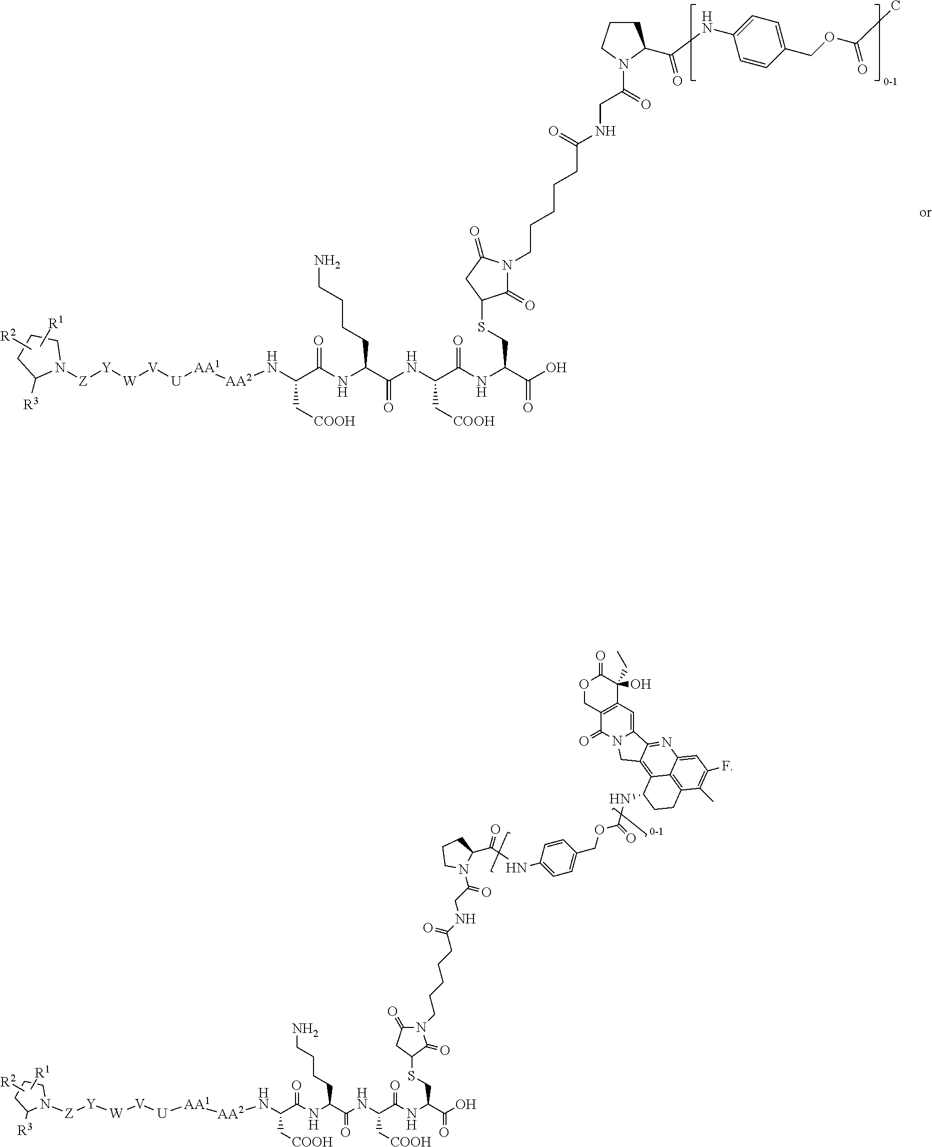

38. (canceled)

39. (canceled)

40. The compound according to claim 37, wherein A or 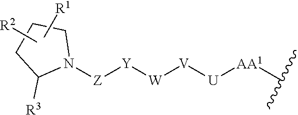
is represented by any one of the following structures.

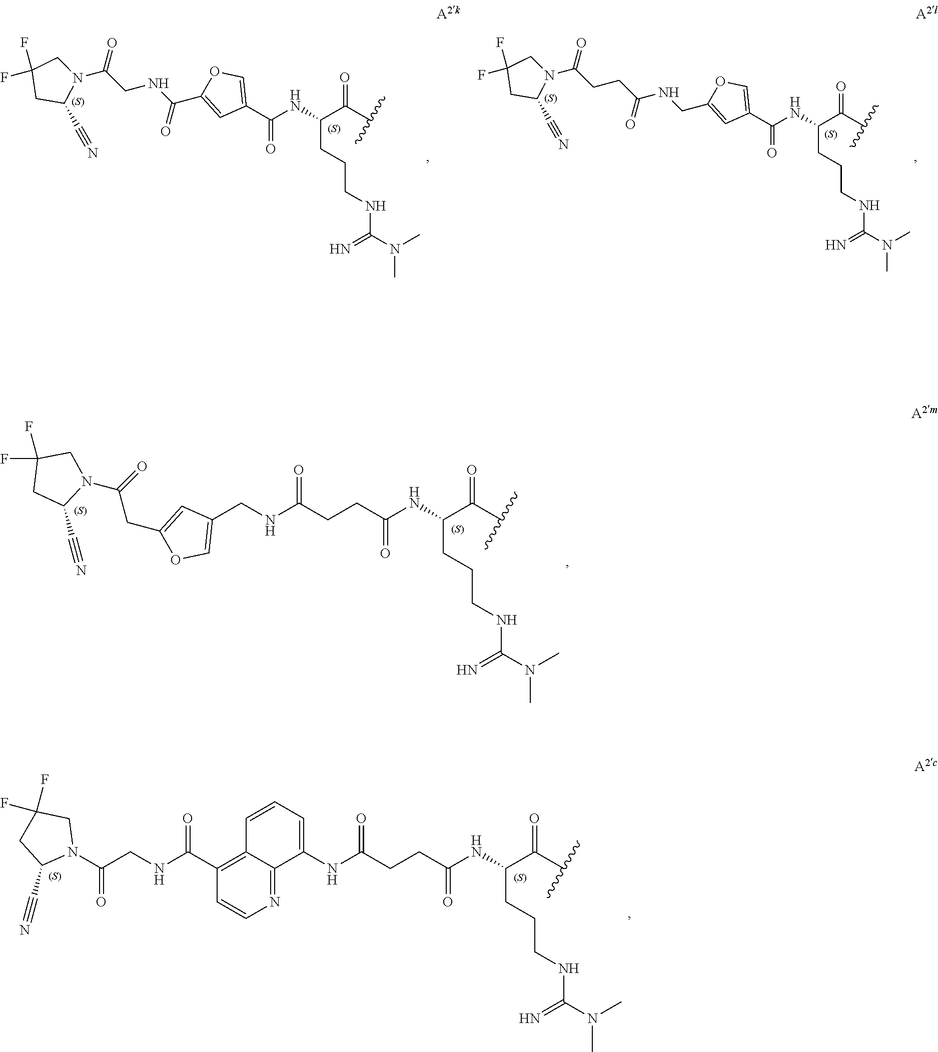

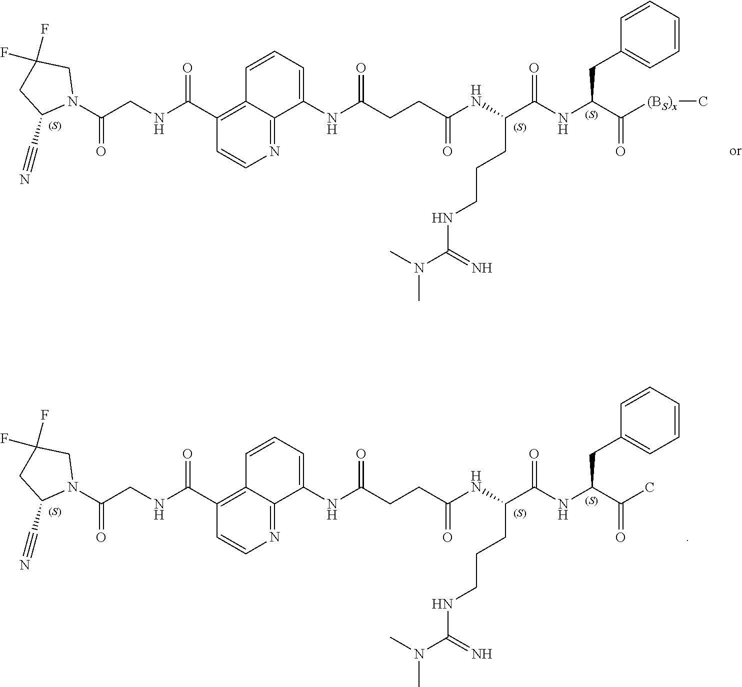

41-46. (canceled)

47. The compound according to claim 1, having a structure selected from the conjugates listed in Table 2 or 4, its individual diastereoisomers, its hydrates, its solvates, its crystal forms, its individual tautomers or a pharmaceutically acceptable salt thereof.

48. A pharmaceutical composition comprising the compound according to claim 1, and a pharmaceutically acceptable excipient.

49-52. (canceled)

53. A method for:
(a) therapy or prophylaxis of a subject suffering from or having risk for a disease or disorder; or
(b) guided surgery practised on a subject suffering from or having risk for a disease or disorder; or
(c) diagnosis of a disease or disorder, the method being practised on the human or animal body and involving a nuclear medicine imaging technique, such as Positron Emission Tomography (PET) or Single Photon Emission Computed Tomography (SPECT); or
(d) targeted delivery of a therapeutic or diagnostic agent to a subject suffering from or having risk for a disease or disorder,
the method comprising administering a therapeutically or diagnostically effective amount of the compound according to claim 1, or a pharmaceutical composition comprising the compound and a pharmaceutically acceptable excipient, to a subject in need thereof.

54. The method according to claim 53, wherein said disease or disorder is independently selected from: inflammation, atherosclerosis, fibrosis, tissue remodeling keloid disorder, cancer, breast cancer, pancreatic cancer, small intestine cancer, colon cancer, multi-drug resistant colon cancer, rectal cancer, colorectal cancer, metastatic colorectal cancer, lung cancer, non-small cell lung cancer, head and neck cancer, ovarian cancer, hepatocellular cancer, oesophageal cancer, hypopharynx cancer, nasopharynx cancer, larynx cancer, myeloma cells, bladder cancer, cholangiocarcinoma, clear cell renal carcinoma, neuroendocrine tumor, oncogenic osteomalacia, sarcoma, CUP (carcinoma of unknown primary), thymus cancer, desmoid tumors, glioma, astrocytoma, cervix cancer, skin cancer, kidney cancer and prostate cancer; and/or
wherein the compound has a prolonged residence at a disease site at a therapeutically relevant level, optionally beyond 1 h, optionally beyond 6 h post injection.


SEQUENCE LISTING (62 sequences)

SEQ ID NO: 1 - protein, 6 aa, synthetic construct
  MOD_RES 1: note = Amino acid at position 1 is dimethylated, and further modified by construct A up to AA1, as shown in Table 4
  REGION 1..6: note = Compound C1
  SITE 3: note = D-Asp
  SITE 4: note = D-Arg
  SITE 5: note = D-Asp
  MOD_RES 6: note = Sidechain modified as shown in Table 4
  SITE 6: note = D-Cys
RFDRDC

SEQ ID NO: 2 - protein, 6 aa, synthetic construct
  MOD_RES 1: note = Amino acid at position 1 is dimethylated, and further modified by construct A up to AA1, as shown in Table 4
  REGION 1..6: note = Compound C2
  SITE 3: note = D-Asp
  SITE 4: note = D-Lys
  SITE 5: note = D-Asp
  MOD_RES 6: note = Sidechain modified as shown in Table 4
  SITE 6: note = D-Cys
RFDKDC

SEQ ID NO: 3 - protein, 6 aa, synthetic construct
  MOD_RES 1: note = Amino acid at position 1 is dimethylated, and further modified by construct A up to AA1, as shown in Table 4
  REGION 1..6: note = Compound C3
  SITE 3: note = D-Asp
  SITE 4: note = D-Arg
  SITE 5: note = D-Asp
  MOD_RES 6: note = Sidechain modified as shown in Table 4
  SITE 6: note = D-Cys
RFDRDC

SEQ ID NO: 4 - protein, 6 aa, synthetic construct
  MOD_RES 1: note = Amino acid at position 1 is dimethylated, and further modified by construct A up to AA1, as shown in Table 4
  REGION 1..6: note = Compound C4
  SITE 3: note = D-Asp
  SITE 4: note = D-Arg
  SITE 5: note = D-Asp
  MOD_RES 6: note = Sidechain modified as shown in Table 4
  SITE 6: note = D-Lys
RFDRDK

SEQ ID NO: 5 - protein, 6 aa, synthetic construct
  MOD_RES 1: note = Amino acid at position 1 is dimethylated, and further modified by construct A up to AA1, as shown in Table 4
  REGION 1..6: note = Compound C5
  SITE 3: note = D-Asp
  SITE 4: note = D-Arg
  SITE 5: note = D-Asp
  MOD_RES 6: note = Sidechain modified as shown in Table 4
RFDRDG

SEQ ID NO: 6 - protein, 6 aa, synthetic construct
  MOD_RES 1: note = Amino acid at position 1 is dimethylated, and further modified by construct A up to AA1, as shown in Table 4
  REGION 1..6: note = Compound C6
  SITE 3: note = D-Asp
  SITE 4: note = D-Arg
  SITE 5: note = D-Asp
RFDRDG

SEQ ID NO: 7 - protein, 6 aa, synthetic construct
  MOD_RES 1: note = Amino acid at position 1 is dimethylated, and further modified by construct A up to AA1, as shown in Table 4
  REGION 1..6: note = Compound C7
  SITE 3: note = D-Asp
  SITE 4: note = D-Lys
  SITE 5: note = D-Asp
  SITE 6: note = D-Cys
RFDKDC

SEQ ID NO: 8 - protein, 6 aa, synthetic construct
  MOD_RES 1: note = Amino acid at position 1 is dimethylated, and further modified by construct A up to AA1, as shown in Table 4
  REGION 1..6: note = Compound C15
  MOD_RES 6: note = Sidechain modified as shown in Table 4
RFDKDC

SEQ ID NO: 9 - protein, 6 aa, synthetic construct
  MOD_RES 1: note = Amino acid at position 1 is dimethylated, and further modified by construct A up to AA1, as shown in Table 4
  REGION 1..6: note = Compound C16
  MOD_RES 6: note = Sidechain modified as shown in Table 4
RFDKDC

SEQ ID NO: 10 - protein, 6 aa, synthetic construct
  MOD_RES 1: note = Amino acid at position 1 is dimethylated, and further modified by construct A up to AA1, as shown in Table 4
  REGION 1..6: note = Compound C17
  MOD_RES 6: note = Sidechain modified as shown in Table 4
RFDKDC

SEQ ID NO: 11 - protein, 6 aa, synthetic construct
  MOD_RES 1: note = Amino acid at position 1 is dimethylated, and further modified by construct A up to AA1, as shown in Table 4
  REGION 1..6: note = Compound C20
  MOD_RES 6: note = Sidechain modified as shown in Table 4
RFDKDC

SEQ ID NO: 12 - protein, 6 aa, synthetic construct
  MOD_RES 1: note = Amino acid at position 1 is dimethylated, and further modified by construct A up to AA1, as shown in Table 4
  REGION 1..6: note = Compound C21
  MOD_RES 6: note = Sidechain modified as shown in Table 4
RFDKDC

SEQ ID NO: 13 - protein, 6 aa, synthetic construct
  MOD_RES 1: note = Amino acid at position 1 is dimethylated, and further modified by construct A up to AA1, as shown in Table 4
  REGION 1..6: note = Compound C22
  SITE 4: note = X in position 4 is HomoArg
  MOD_RES 6: note = Sidechain modified as shown in Table 4
RFDXDC

SEQ ID NO: 14 - protein, 6 aa, synthetic construct
  MOD_RES 1: note = Amino acid at position 1 is dimethylated, and further modified by construct A up to AA1, as shown in Table 4
  REGION 1..6: note = Compound C23
RFDRDC

SEQ ID NO: 15 - protein, 6 aa, synthetic construct
  MOD_RES 1: note = Amino acid at position 1 is dimethylated, and further modified by construct A up to AA1, as shown in Table 4
  REGION 1..6: note = Compound C24
  MOD_RES 6: note = Sidechain modified as shown in Table 4
RFDKDC

SEQ ID NO: 16 - protein, 6 aa, synthetic construct
  MOD_RES 1: note = Amino acid at position 1 is dimethylated, and further modified by construct A up to AA1, as shown in Table 4
  REGION 1..6: note = Compound C25
  MOD_RES 6: note = Sidechain modified as shown in Table 4
RFDKDK

SEQ ID NO: 17 - protein, 6 aa, synthetic construct
  MOD_RES 1: note = Amino acid at position 1 is dimethylated, and further modified by construct A up to AA1, as shown in Table 4
  REGION 1..6: note = Compound C26
  MOD_RES 6: note = Sidechain modified as shown in Table 4
RFDRDG

SEQ ID NO: 18 - protein, 6 aa, synthetic construct
  MOD_RES 1: note = Amino acid at position 1 is dimethylated, and further modified by construct A up to AA1, as shown in Table 4
  REGION 1..6: note = Compound C27
  MOD_RES 6: note = Sidechain modified as shown in Table 4
RFDRDC

SEQ ID NO: 19 - protein, 6 aa, synthetic construct
  MOD_RES 1: note = Amino acid at position 1 is dimethylated, and further modified by construct A up to AA1, as shown in Table 4
  REGION 1..6: note = Compound C28
  MOD_RES 6: note = Sidechain modified as shown in Table 4
RFDKDC

SEQ ID NO: 20 - protein, 6 aa, synthetic construct
  MOD_RES 1: note = Amino acid at position 1 is dimethylated, and further modified by construct A up to AA1, as shown in Table 4
  REGION 1..6: note = Compound C31
  MOD_RES 6: note = Sidechain modified as shown in Table 4
RFDKDC

SEQ ID NO: 21 - protein, 6 aa, synthetic construct
  MOD_RES 1: note = Amino acid at position 1 is dimethylated, and further modified by construct A up to AA1, as shown in Table 4
  REGION 1..6: note = Compound C32
  MOD_RES 6: note = Sidechain modified as shown in Table 4
RFDKDC

SEQ ID NO: 22 - protein, 5 aa, synthetic construct
  MOD_RES 1: note = Amino acid at position 1 is dimethylated, and further modified by construct A up to AA1, as shown in Table 4
  REGION 1..5: note = Compound C33
  SITE 3: note = D-Glu
  SITE 4: note = D-Glu
  MOD_RES 5: note = Sidechain modified as shown in Table 4
RFEEK

SEQ ID NO: 23 - protein, 5 aa, synthetic construct
  MOD_RES 1: note = Amino acid at position 1 is dimethylated, and further modified by construct A up to AA1, as shown in Table 4
  SITE 3: note = X in position 3 is D-Glu
  SITE 4: note = X in position 4 is D-Glu
  MOD_RES 5: note = Sidechain modified as shown in Table 4
  REGION 1..5: note = Compound C34
  MOD_RES 5: note = Sidechain modified as shown in Table 4
RFEEK

SEQ ID NO: 24 - protein, 6 aa, synthetic construct
  MOD_RES 1: note = Amino acid at position 1 is dimethylated, and further modified by construct A up to AA1, as shown in Table 4
  REGION 1..6: note = Compound C49
  MOD_RES 6: note = Sidechain modified as shown in Table 4
RFDKDC

SEQ ID NO: 25 - protein, 6 aa, synthetic construct
  MOD_RES 1: note = Amino acid at position 1 is dimethylated, and further modified by construct A up to AA1, as shown in Table 4
  REGION 1..6: note = Compound C49.1
  MOD_RES 6: note = Sidechain modified as shown in Table 4
RFDKDC

SEQ ID NO: 26 - protein, 6 aa, synthetic construct
  MOD_RES 1: note = Amino acid at position 1 is dimethylated, and further modified by construct A up to AA1, as shown in Table 4
  REGION 1..6: note = Compound C49.2
  MOD_RES 6: note = Sidechain modified as shown in Table 4
RFDKDC

SEQ ID NO: 27 - protein, 6 aa, synthetic construct
  MOD_RES 1: note = Amino acid at position 1 is dimethylated, and further modified by construct A up to AA1, as shown in Table 4
  REGION 1..6: note = Compound C49.3
  MOD_RES 6: note = Sidechain modified as shown in Table 4
RFDKDC

SEQ ID NO: 28 - protein, 6 aa, synthetic construct
  MOD_RES 1: note = Amino acid at position 1 is dimethylated, and further modified by construct A up to AA1, as shown in Table 4
  REGION 1..6: note = Compound C49.4
  MOD_RES 6: note = Sidechain modified as shown in Table 4
RFDKDC

SEQ ID NO: 29 - protein, 6 aa, synthetic construct
  MOD_RES 1: note = Amino acid at position 1 is dimethylated, and further modified by construct A up to AA1, as shown in Table 4
  REGION 1..6: note = Compound C49.5
  MOD_RES 6: note = Sidechain modified as shown in Table 4
RFDKDC

SEQ ID NO: 30 - protein, 6 aa, synthetic construct
  MOD_RES 1: note = Amino acid at position 1 is dimethylated, and further modified by construct A up to AA1, as shown in Table 4
  REGION 1..6: note = Compound C49.6
  MOD_RES 6: note = Sidechain modified as shown in Table 4
RFDKDC

SEQ ID NO: 31 - protein, 6 aa, synthetic construct
  MOD_RES 1: note = Amino acid at position 1 is dimethylated, and further modified by construct A up to AA1, as shown in Table 4
  REGION 1..6: note = Compound C50
  MOD_RES 6: note = Sidechain modified as shown in Table 4
RFDKDC

SEQ ID NO: 32 - protein, 6 aa, synthetic construct
  MOD_RES 1: note = Amino acid at position 1 is dimethylated, and further modified by construct A up to AA1, as shown in Table 4
  REGION 1..6: note = Compound C50.1
  MOD_RES 6: note = Sidechain modified as shown in Table 4
RFDKDC

SEQ ID NO: 33 - protein, 6 aa, synthetic construct
  MOD_RES 1: note = Amino acid at position 1 is dimethylated, and further modified by construct A up to AA1, as shown in Table 4
  REGION 1..6: note = Compound C50.2
  MOD_RES 6: note = Sidechain modified as shown in Table 4
RFDKDC

SEQ ID NO: 34 - protein, 6 aa, synthetic construct
  MOD_RES 1: note = Amino acid at position 1 is dimethylated, and further modified by construct A up to AA1, as shown in Table 4
  REGION 1..6: note = Compound C50.3
  MOD_RES 6: note = Sidechain modified as shown in Table 4
RFDKDC

SEQ ID NO: 35 - protein, 6 aa, synthetic construct
  MOD_RES 1: note = Amino acid at position 1 is dimethylated, and further modified by construct A up to AA1, as shown in Table 4
  REGION 1..6: note = Compound C51
  MOD_RES 6: note = Sidechain modified as shown in Table 4
RFDKDV

SEQ ID NO: 36 - protein, 6 aa, synthetic construct
  MOD_RES 1: note = Amino acid at position 1 is dimethylated, and further modified by construct A up to AA1, as shown in Table 4
  REGION 1..6: note = Compound C51.1
  MOD_RES 6: note = Sidechain modified as shown in Table 4
RFDKDV

SEQ ID NO: 37 - protein, 6 aa, synthetic construct
  MOD_RES 1: note = Amino acid at position 1 is dimethylated, and further modified by construct A up to AA1, as shown in Table 4
  REGION 1..6: note = Compound C51.2
  MOD_RES 6: note = Sidechain modified as shown in Table 4
RFDKDV

SEQ ID NO: 38 - protein, 6 aa, synthetic construct
  MOD_RES 1: note = Amino acid at position 1 is dimethylated, and further modified by construct A up to AA1, as shown in Table 4
  REGION 1..6: note = Compound C51.3
  MOD_RES 6: note = Sidechain modified as shown in Table 4
RFDKDV

SEQ ID NO: 39 - protein, 6 aa, synthetic construct
  MOD_RES 1: note = Amino acid at position 1 is dimethylated, and further modified by construct A up to AA1, as shown in Table 4
  REGION 1..6: note = Compound C55
  MOD_RES 6: note = Sidechain modified as shown in Table 4
RFDKDC

SEQ ID NO: 40 - protein, 6 aa, synthetic construct
  MOD_RES 1: note = Amino acid at position 1 is dimethylated, and further modified by construct A up to AA1, as shown in Table 4
  REGION 1..6: note = Compound C57
  MOD_RES 6: note = Sidechain modified as shown in Table 4
RFDKDC

SEQ ID NO: 41 - protein, 6 aa, synthetic construct
  MOD_RES 1: note = Amino acid at position 1 is dimethylated, and further modified by construct A up to AA1, as shown in Table 4
  REGION 1..6: note = Compound C58
  MOD_RES 6: note = Sidechain modified as shown in Table 4
RFDKDC

SEQ ID NO: 42 - protein, 6 aa, synthetic construct
  MOD_RES 1: note = Amino acid at position 1 is dimethylated, and further modified by construct A up to AA1, as shown in Table 4
  REGION 1..6: note = Compound C59
  MOD_RES 6: note = Sidechain modified as shown in Table 4
RFDKDC

SEQ ID NO: 43 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 line of Description of the PCT application as filed
  REGION 1..6: note = Claim 29(d) column 1 line 2 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDRDC

SEQ ID NO: 44 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the description of the PCT application as filed
  REGION 1..6: note = Claim 29(d) column 1 line 3 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDRDG

SEQ ID NO: 45 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 line 20 of the description of the PCT application as filed
  REGION 1..6: note = Claim 29 (d) column 1 line 4 of the PCT application as filed
  SITE 2: note = X in position 2 is AA2 as defined on p. 4 lines 10-15 of the description of the PCT application as filed
  MOD_RES 6: note = Sidechain modified as shown in claim 29 of the PCT application as filed
XXDKDK

SEQ ID NO: 46 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 1 line 5 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDKDV

SEQ ID NO: 47 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 1 line 6 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDRDC

SEQ ID NO: 48 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 1 line 7 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDRDC

SEQ ID NO: 49 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 1 line 8 of the PCT application as filed
  SITE 2: note = X in position 2 is AA2 as defined on p. 3 of the description of the PCT application as filed
  SITE 4: note = HomoArg
  MOD_RES 6: note = Sidechain modified as shown in claim 29 of the PCT application as filed
XXDRDC

SEQ ID NO: 50 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 1 line 9 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDKDC

SEQ ID NO: 51 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 2 line 2 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDKDC

SEQ ID NO: 52 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d), column 2 line 3 of the PCT application as filed
  SITE 2: note = X in position 2 is AA2 as defined on p. 3 f the description of the PCT application as filed
  MOD_RES 6: note = Sidechain modified as shown in claim 29 of the PCT application as filed
XXDKDC

SEQ ID NO: 53 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d), column 2 line 4 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDKDC

SEQ ID NO: 54 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 2 line 5 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDKDC

SEQ ID NO: 55 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d), page 120, column 2 line 6 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDKDC

SEQ ID NO: 56 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 2 line 7 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDKDC

SEQ ID NO: 57 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 2 line 8
  SITE 2: note = X in position 2 is AA2 as defined on p. 3 of the description of the PCT application as filed
  MOD_RES 6: note = Sidechain modified as shown in claim 29 of the PCT application as filed
XXDKDC

SEQ ID NO: 58 - protein, 6 aa, synthetic construct
  MOD_RES 1: note = Amino acid at position 1 is modified by construct A up to AA1, as defined on page 3 of the description of the PCT application as filed
  SITE 1: note = X in position 1 is AA1 as defined on p.3 of the Description of the PCT application as filed
  REGION 1..6: note = Claim 29(d) column 2 line 9 of the PCT application as filed
  SITE 2: note = X in position 2 is AA2 as defined on p. 3 of the description of the PCT application as filed
  MOD_RES 6: note = Sidechain modified as shown in claim 29 of the PCT application as filed
XXDKDC

SEQ ID NO: 59 - skipped

SEQ ID NO: 60 - protein, 4 aa, synthetic construct
  REGION 1..4: note = page 18 line 5-6
DRDC

SEQ ID NO: 61 - protein, 4 aa, synthetic construct
  REGION 1..4: note = page 18 line 6
DKDC

SEQ ID NO: 62 - protein, 6 aa, synthetic construct
CDKDFR